SEQUENCE LISTING (68 sequences)

SEQ ID NO: 1 - DNA, 25 bp, Artificial Sequence
  Synthetic
gtagtttaatgagtttaggtttttt

SEQ ID NO: 2 - DNA, 21 bp, Artificial Sequence
  Synthetic
gttttggagagtatttgtatt

SEQ ID NO: 3 - DNA, 26 bp, Artificial Sequence
  Synthetic
gagtttttttgttttagagtggttga

SEQ ID NO: 4 - DNA, 22 bp, Artificial Sequence
  Synthetic
gggttagggttaggtaggttgt

SEQ ID NO: 5 - DNA, 25 bp, Artificial Sequence
  Synthetic
attttttgtgttggaaaatgaatgt

SEQ ID NO: 6 - DNA, 26 bp, Artificial Sequence
  Synthetic
gaatggagaggaggatgtttatttat

SEQ ID NO: 7 - DNA, 25 bp, Artificial Sequence
  Synthetic
tgtttatttttgtgaatttgttttg

SEQ ID NO: 8 - DNA, 25 bp, Artificial Sequence
  Synthetic
attttgtaagtttatgagttgtggg

SEQ ID NO: 9 - DNA, 25 bp, Artificial Sequence
  Synthetic
tgttttatttttgggttgttttttt

SEQ ID NO: 10 - DNA, 25 bp, Artificial Sequence
  Synthetic
ttatttaagggaagaaagggtattg

SEQ ID NO: 11 - DNA, 25 bp, Artificial Sequence
  Synthetic
tagtttatatgttggaagtggttgg

SEQ ID NO: 12 - DNA, 25 bp, Artificial Sequence
  Synthetic
taggaagagttatagaaagggggtt

SEQ ID NO: 13 - DNA, 25 bp, Artificial Sequence
  Synthetic
ttttttggggtgtttatttttagag

SEQ ID NO: 14 - DNA, 25 bp, Artificial Sequence
  Synthetic
aatagggaggaaggtttgattagat

SEQ ID NO: 15 - DNA, 25 bp, Artificial Sequence
  Synthetic
gtttaagaaagttaggatgggagtg

SEQ ID NO: 16 - DNA, 25 bp, Artificial Sequence
  Synthetic
gagtttttggtaaagagtttaggaa

SEQ ID NO: 17 - DNA, 25 bp, Artificial Sequence
  Synthetic
attgtttaggttggttagtttattt

SEQ ID NO: 18 - DNA, 24 bp, Artificial Sequence
  Synthetic
gtatggtgagattattattggagt

SEQ ID NO: 19 - DNA, 24 bp, Artificial Sequence
  Synthetic
gggttttttagttatttgattagt

SEQ ID NO: 20 - DNA, 24 bp, Artificial Sequence
  Synthetic
gggttttttagttatttgattagt

SEQ ID NO: 21 - DNA, 25 bp, Artificial Sequence
  Synthetic
tttgtttttgtttttatttgatttt

SEQ ID NO: 22 - DNA, 20 bp, Artificial Sequence
  Synthetic
tcagttctcgggtcctggag

SEQ ID NO: 23 - DNA, 22 bp, Artificial Sequence
  Synthetic
gtgtcactggagacagaatgga

SEQ ID NO: 24 - DNA, 22 bp, Artificial Sequence
  Synthetic
agggtgtcactggagacagaat

SEQ ID NO: 25 - DNA, 22 bp, Artificial Sequence
  Synthetic
ctgaacgggaagctcactggca

SEQ ID NO: 26 - DNA, 20 bp, Artificial Sequence
  Synthetic
gacttgctgctgatccatga

SEQ ID NO: 27 - DNA, 21 bp, Artificial Sequence
  Synthetic
ccttgttgtccaacttctccc

SEQ ID NO: 28 - DNA, 22 bp, Artificial Sequence
  Synthetic
gagtcaaaggcaagtgaaggtg

SEQ ID NO: 29 - DNA, 20 bp, Artificial Sequence
  Synthetic
gacctctgtggcgacttcat

SEQ ID NO: 30 - DNA, 21 bp, Artificial Sequence
  Synthetic
aggtggccaacattagagtcc

SEQ ID NO: 31 - DNA, 22 bp, Artificial Sequence
  Synthetic
cttctttcgaaatgacctggag

SEQ ID NO: 32 - DNA, 21 bp, Artificial Sequence
  Synthetic
ctacacccaaatttccattac

SEQ ID NO: 33 - DNA, 25 bp, Artificial Sequence
  Synthetic
aaactcttttaaactaaccataaac

SEQ ID NO: 34 - DNA, 25 bp, Artificial Sequence
  Synthetic
acaactaaaattacttctccacacc

SEQ ID NO: 35 - DNA, 25 bp, Artificial Sequence
  Synthetic
acacctccattctatctccaataac

SEQ ID NO: 36 - DNA, 26 bp, Artificial Sequence
  Synthetic
aaacctaactaaaaaactaaaccatc

SEQ ID NO: 37 - DNA, 25 bp, Artificial Sequence
  Synthetic
aaaaacaaccataccaaactcaaac

SEQ ID NO: 38 - DNA, 25 bp, Artificial Sequence
  Synthetic
acctataaataaaaaaaacccaacc

SEQ ID NO: 39 - DNA, 25 bp, Artificial Sequence
  Synthetic
ttctattcctatatccctctatccc

SEQ ID NO: 40 - DNA, 25 bp, Artificial Sequence
  Synthetic
cacacaccactactcacctacaaac

SEQ ID NO: 41 - DNA, 25 bp, Artificial Sequence
  Synthetic
atttaaacaaaaatccaaaacctac

SEQ ID NO: 42 - DNA, 25 bp, Artificial Sequence
  Synthetic
aaaacccctaaaaaaactctacacc

SEQ ID NO: 43 - DNA, 25 bp, Artificial Sequence
  Synthetic
cattaacaaaattccaaacatcaaa

SEQ ID NO: 44 - DNA, 20 bp, Artificial Sequence
  Synthetic
aaccctcaatctctcccaac

SEQ ID NO: 45 - DNA, 25 bp, Artificial Sequence
  Synthetic
aaaccctaataacactaaacccaac

SEQ ID NO: 46 - DNA, 27 bp, Artificial Sequence
  Synthetic
aaaaaaccaaataaaaacacctaaaac

SEQ ID NO: 47 - DNA, 24 bp, Artificial Sequence
  Synthetic
caaatctaaaaacaaaaaaaaccc

SEQ ID NO: 48 - DNA, 25 bp, Artificial Sequence
  Synthetic
cttttccctaactacccatcaatta

SEQ ID NO: 49 - DNA, 24 bp, Artificial Sequence
  Synthetic
actaaatccaaaaccctaaacatc

SEQ ID NO: 50 - DNA, 22 bp, Artificial Sequence
  Synthetic
caaaatctctctaaaccaactc

SEQ ID NO: 51 - DNA, 21 bp, Artificial Sequence
  Synthetic
aaaaccctcctaaacctctac

SEQ ID NO: 52 - DNA, 25 bp, Artificial Sequence
  Synthetic
attactcctacctccaaacctttac

SEQ ID NO: 53 - DNA, 19 bp, Artificial Sequence
  Synthetic
tccttggctacccttggac

SEQ ID NO: 54 - DNA, 22 bp, Artificial Sequence
  Synthetic
tagattcacatcagccatctgc

SEQ ID NO: 55 - DNA, 20 bp, Artificial Sequence
  Synthetic
ctacccttggaccccatttc

SEQ ID NO: 56 - DNA, 23 bp, Artificial Sequence
  Synthetic
ttactccttggaggccatgtggg

SEQ ID NO: 57 - DNA, 21 bp, Artificial Sequence
  Synthetic
aggttcacgtactcaggggag

SEQ ID NO: 58 - DNA, 22 bp, Artificial Sequence
  Synthetic
acagtggcttctcctctgtgat

SEQ ID NO: 59 - DNA, 22 bp, Artificial Sequence
  Synthetic
acagtggcttctcctctgtgat

SEQ ID NO: 60 - DNA, 19 bp, Artificial Sequence
  Synthetic
ggcaggtgaacttgcaatc

SEQ ID NO: 61 - DNA, 19 bp, Artificial Sequence
  Synthetic
aacagtccaggcagacgag

SEQ ID NO: 62 - DNA, 19 bp, Artificial Sequence
  Synthetic
tccgagtccgagtcctctt

SEQ ID NO: 63 - DNA, 25 bp, Artificial Sequence
  Synthetic
tgcaatggcctgtagtccccctagt

SEQ ID NO: 64 - DNA, 26 bp, Artificial Sequence
  Synthetic
agccttcgaggtgcagagtgtgtgct

SEQ ID NO: 65 - DNA, 25 bp, Artificial Sequence
  Synthetic
ccaggggcatccacatgaacccgca

SEQ ID NO: 66 - DNA, 24 bp, Artificial Sequence
  Synthetic
cgctgggcgtttgggtttgggggt

SEQ ID NO: 67 - DNA, 21 bp, Artificial Sequence
  Synthetic
cagagccatacctggctacac

SEQ ID NO: 68 - DNA, 24 bp, Artificial Sequence
  Synthetic
gccgcaggggctgctcatcatcca

(19) United States
(12) Patent Application Publication
(10) Pub. No.: US 2013/0022974 A1
(43) Pub. Date: Jan. 24, 2013

(54) DNA METHYLATION PROFILES IN CANCER

(71) Applicants: Arul M. Chinnaiyan, Plymouth, MI (US); Mohan Saravana Dhanasekaran, Ann Arbor, MI (US); Jung Kim, Northville, MI (US)

(72) Inventors: Arul M. Chinnaiyan, Plymouth, MI (US); Mohan Saravana Dhanasekaran, Ann Arbor, MI (US); Jung Kim, Northville, MI (US)

(73) Assignee: THE REGENTS OF THE UNIVERSITY OF MICHIGAN, Ann Arbor, MI (US)

(21) Appl. No.: 13/523,545

(22) Filed: Jun. 14, 2012

Related U.S. Application Data
(60) Provisional application No. 61/498,296, filed on Jun. 17, 2011.

(51) Int. Cl. C12Q 1/68

(52) U.S. Cl. 435/6.11

(57) ABSTRACT

The present invention relates to compositions and methods for cancer diagnosis, research and therapy, including but not limited to, cancer markers. In particular, the present invention relates to methylation levels of genes (e.g., in CGI islands of the promoter regions) as diagnostic markers and clinical targets for prostate cancer.

### CROSS REFERENCE TO RELATED APPLICATIONS

This application claims priority to U.S. Provisional Application No. 61/498,296, filed Jun. 17, 2011, which is herein incorporated by reference in its entirety.

### STATEMENT REGARDING FEDERALLY SPONSORED RESEARCH OR DEVELOPMENT

This invention was made with government support under CA069568, CA132874, CA111275 and DA021519 awarded by the National Institutes of Health and BC07023 awarded by the Army Medical Research and Material Command. The government has certain rights in the invention.

### FIELD OF THE INVENTION

The present invention relates to compositions and methods for cancer diagnosis, research and therapy, including but not limited to, cancer markers. In particular, the present invention relates to methylation levels of genes (e.g., in CGI islands of the promoter regions) as diagnostic markers and clinical targets for prostate cancer.

### BACKGROUND OF THE INVENTION

A central aim in cancer research is to identify altered genes that are causally implicated in oncogenesis. Several types of somatic mutations have been identified including base substitutions, insertions, deletions, translocations, and chromosomal gains and losses, all of which result in altered activity of an oncogene or tumor suppressor gene. First hypothesized in the early 1900's, there is now compelling evidence for a causal role for chromosomal rearrangements in cancer (Rowley, Nat Rev Cancer 1: 245 (2001)). Recurrent chromosomal aberrations were thought to be primarily characteristic of leukemias, lymphomas, and sarcomas. Epithelial tumors (carcinomas), which are much more common and contribute to a relatively large fraction of the morbidity and mortality associated with human cancer, comprise less than 1% of the known, disease-specific chromosomal rearrangements (Mitelman, Mutat Res 462: 247 (2000)). While hematological malignancies are often characterized by balanced, disease-specific chromosomal rearrangements, most solid tumors have a plethora of non-specific chromosomal aberrations. It is thought that the karyotypic complexity of solid tumors is due to secondary alterations acquired through cancer evolution or progression.

Two primary mechanisms of chromosomal rearrangements have been described. In one mechanism, promoter/enhancer elements of one gene are rearranged adjacent to a proto-oncogene, thus causing altered expression of an oncogenic protein. This type of translocation is exemplified by the apposition of immunoglobulin (IG) and T-cell receptor (TCR) genes to MYC leading to activation of this oncogene in B- and T-cell malignancies, respectively (Rabbitts, Nature 372: 143 (1994)). In the second mechanism, rearrangement results in the fusion of two genes, which produces a fusion protein that may have a new function or altered activity. The prototypic example of this translocation is the BCR-ABL gene fusion in chronic myelogenous leukemia (CML) (Rowley, Nature 243: 290 (1973); de Klein et al., Nature 300: 765 (1982)). Importantly, this finding led to the rational development of imatinib mesylate (Gleevec), which successfully targets the BCR-ABL kinase (Deininger et al., Blood 105: 2640 (2005)). Thus, diagnostic methods that specifically identify epithelial tumors are needed.

### SUMMARY OF THE INVENTION

The present invention relates to compositions and methods for cancer diagnosis, research and therapy, including but not limited to, cancer markers. In particular, the present invention relates to methylation levels of genes (e.g., in CGI islands of the promoter regions) as diagnostic markers and clinical targets for prostate cancer.

Embodiments of the present invention provide compositions, kits, and methods useful in the detection and screening of prostate cancer. Experiments conducted during the course of development of embodiments of the present invention identified aberent methylation status of certain genes in prostate cancer. Some embodiments of the present invention provide compositions and methods for detecting such aberently methylated genes. Identification of aberently methylated genes finds use in screening, diagnostic and research uses.

For example, in some embodiments, the present invention provides compositions, kits and methods of screening for the presence of prostate cancer in a subject, comprising contacting a biological sample from a subject with a reagent for detecting the methylation status of one or more genes (e.g., including but not limited to, WFDC2, MAGI2, MEIS2, NTN4, GPRC5B, C9orf125, FGFR2, AOX1, VAMP5, C14orf159, PPP1R3C, S100A16 or AMT as well as one or more of the genes listed in Table 4; and detecting the methylation status of said genes using an in vitro assay, wherein a higher degress of methylation of said genes in said sample relative to the level of methylation in normal prostate cells in indicative of prostate cancer in said subject. In some embodiments, the sample is selected from tissue, blood, plasma, serum, urine, urine supernatant, urine cell pellet, semen, prostatic secretions or prostate cells. In some embodiments, the detecting the level of methylation is carried out utilizing Methylplex-Next Generation Sequencing (M-NGS) or another suitable assay. In some embodiments, the cancer is localized prostate cancer or metastatic prostate cancer. In some embodiments, the methylation is detected in the 5′ non-coding region (e.g., promoter region) of the gene. In some embodiments, expression of genes is decreased when an increased level of methylation is present. In some embodiments, analysis is conducted using a computer implemented method, and results are displayed to a user using a user interface. In some embodiments, the results of the method are used to determine a treatment course of action. In some embodiments, the treatment course of action (e.g., chemotherapy) is administered. In some embodiments, the method is performed again after treatment and is used to determine whether further treatment is needed and/or administered.

Additional embodiments are described herein.

### DESCRIPTION OF THE FIGURES

FIG. 1 shows characterization of genome-wide methylation patterns in prostate cells by M-NGS (A) Venn diagram represents a 70% overlap between the regions methylated in LNCaP and PrEC cells. (B) In LNCaP and PrEC, the majority of DNA methylation occurred in intergenic and intronic regions and the genomic distribution of methylation peaks was similar. (C) Promoter associated CpG islands displayed 7 fold difference in methylation between LNCaP and PrEC cells. (D) DNA methylation in APC, CHMP4A, CALML3, CDKN2A, KCTD1, LAMC2, RASSF1, SHC1, TINAGL1 and TSPAN1 gene promoters in LNCaP (L) cells; SPON2 in PrEC (P) cells and a negative control region in MYC were validated by bisulfite sequencing.

FIG. 2 shows DNA methylation pattern in prostate tissues (A) Genome-wide distribution of DNA methylation in various prostate sample groups analyzed. The majority of methylation peaks are confined to intergenic and intronic regions similar to cell lines. (B) A gradual increase in percent methylation with cancer progression among promoter CGIs compared CGIs located in other genomic regions was observed. * Pearson's Chi-squared test p-value <2×10⁻¹⁶

FIG. 3 shows promoter DNA methylation during prostate cancer progression A total of 6619 gene promoters from 6077 unique RefSeq genes harbored DNA methylation among the various sample groups analyzed (Normal, Benign Adjacent, PCa or MET). Each row represents a unique promoter region at 100base pair window size, covering ±1500 bp flanking the transcription start site, indicated by white dotted line. The location of CpG island in methylated gene promoters is shown in first column. Promoters are ordered by the location of methylation on CpG island, adjacent to the island (shores) or on promoters that lacked CpG islands for groups I to IV. Methylation patterns in prostate cells PrEC and LNCaP are presented alongside for comparison.

FIG. 4 shows that promoter methylation is associated with gene repression.

FIG. 5 shows WFDC2, TACSTD2 and GSTP1 methylation in prostate tissue panel. MethylProfiler qPCR was used to determine DNA methylation of the (A) WFDC2 (B) TACSTD2 and GSTP1 genes. 17/22 prostate cancer tissues and 6/6 transformed prostate cell lines showed methylation of WFDC2 promoter, whereas there was no detectable methylation in normal (0/3), benign adjacent tissues (0/7) or the normal PrEC cells. (B) Methylation of TACSTD2 promoter in prostate tissues and cell lines were assessed by MethylProfiler qPCR. Twenty one percent cancer tissues (5/23) and prostate cancer cell lines, VCaP, LNCaP and PC3 were methylated. (C) Methylprofiler qPCR analysis of GSTP1. 20/22 prostate cancer tissues, 1/7 benign adjacent tissues and 6/6 transformed prostate cell lines showed methylation of GSTP1 promoter, whereas there was no detectable methylation in normal tissues (0/3), or normal PrEC cells.

FIG. 6 shows regulation of alternate transcription start site utilization by DNA methylation (A, D) Cancer-specific DNA methylation enables switching of alternative transcriptional start sites (TSS) leading to transcript isoform regulation. In RASSF1 (A) and NDRG2 (D), CpG methylation occurs at the TSS of the longer variants, with H3K4me3 marks positioned on the TSS of the shorter variants. (B, E) Preferential silencing and 5-Aza-induced re-expression of CpG-methylated variants in LNCaP cells. (B, D) 5′RACE results validated RASSF1 variant-3 and NDRG2 variants 5-8 expression in LNCaP cells. (C, F) Exon expression values from LNCaP RNA-Seq data, supports the corresponding variant transcription of RASSF1 and NDRG2 genes.

FIG. 7 shows mutually exclusive patterns of promoter DNA methylation and histone H3K4me3 marks in LNCaP cells.

FIG. 8 shows differentially methylated regions between ETS-Positive and ETS-Negative samples. (A) Venn diagram displays the methylation overlap observed between ETS-Positive, ETS-Negative and benign prostate tissue samples. (B) The coverage for various repeat elements was higher in ETS-Positive compared to ETS-Negative samples indicating higher methylation in the former. The fold difference for methylation in each class of repeat element is indicated by the line plot above. (C) Percent methylation was assessed independently by pyro-sequencing assays for LINE-1 elements and GSTP1 gene promoter methylation in prostate tissue panel (Benign n=5, ETS-Positive cancers n=10 and ETS-Negative cancers n=4).

FIG. 9 shows a schematic of M-NGS library generation.

FIG. 10 shows that regression analysis of M-NGS mapped reads and HMM output shows high correlation between sequencing runs. (A) Reads that mapped to chromosome 21 in LNCaP400 bp-1 and -5, and PrEC400 bp-1 and -5 runs were compared using the window size of 25 bp. In LNCaP samples, a total of 33,627 reads were present at 25 bp windows with R² value of 0.9508, and in PrEC, 37,406 reads with R² value of 0.8556 was observed. (B) Linear regression analysis of all DNA methylation that occurred on CGIs showed high correlation (R² value=0.9398 and 0.9819, n=5,734 and 4,966, respectively).

FIG. 11 shows a correlation between M-NGS vs Methylplex-Array and M-NGS vs MeDIP-Seq results. (A) Methylplex-array libraries made from LNCaP (Cy5) and PrEC (Cy3) cells were hybridized to Agilent human CGI microarray. Array results are displayed on the left in heatmap form. (B) Overlap in methylated CpG islands located within or outside gene promoters (1500 bps flanking the transcription start site) in LNCaP cells identified by M-NGS and MeDIP-Seq.

FIG. 12 shows promoter methylation in LNCaP and PrEC cells identified by M-NGS.

FIG. 13 shows representations of Methylplex NGS sequencing data used for nomination of methylated candidate gene promoters.

FIG. 14 shows representations of Methylplex NGS sequencing data used for nomination of methylated candidate gene promoters.

FIG. 15 shows representations of Methylplex NGS sequencing data used for nomination of methylated candidate gene promoters.

FIG. 16 shows DNA methylation in AOX1, C9orf125, NTN4, AMT, PPP1R3C and NAP1L5 gene promoters in LNCaP (L), PrEC (P), Universally methylated control DNA (MC-DNA, ZymoResearch Inc) and Unmethylated control Fetal DNA (UMC-DNA, Millipore Inc) were validated by bisulfite sequencing.

FIG. 17 shows that significance analysis of microarray (SAM) identified re-expression of LNCaP methylated genes after 5-Aza treatment.

FIG. 18 shows that genes hypermethylated in LNCaP cells are enriched for biologically-significant concepts. (A) Molecular Concept Map (MCM) analysis of LNCaP methylated genes revealed enrichment of gene signatures repressed in prostate cancer and over-expressed in benign prostate tissues from multiple studies. (B) MCM analysis of PrEC methylated genes shows the enrichment only for histone modification concept.

FIG. 19 shows gene set enrichment analysis (GSEA) showing the association between gene repression and promoter methylation. (A) Methylated genes from LNCaP and PrEC cells were tested for their corresponding ranked gene expression in next generation transcriptomic sequencing (RNA-Seq). (B) While no significant association was observed between gene body methylation and gene expression in LNCaP (p-value <0.623), candidates with gene promoter methylation with and without the presence of CGIs in LNCaP cells are enriched with under-expressed genes (p-value <0.0039 and 0.0015, respectively).

FIG. 20 shows that DNA methylation is associated with gene repression.

FIG. 21 shows methylprofiler PCR-bisulfite sequencing comparison in PCa panel.

FIG. 22 shows that cancer-specific DNA methylation enables switching of alternative transcriptional start sites (TSS) leading to transcript isoform regulation. Figure details characterization of APC gene similar to the data presented for RASSF1 and NDRG2 (FIG. 6). (A) In contrast to the two examples presented in FIG. 6, APC exhibits the reverse, with H3K4me3 on the longer variant 1 and CpG methylation at the shorter variants 2 and 3. (B) Preferential silencing and 5-Aza-induced re-expression of CpG-methylated variants of APC in LNCaP cells. (C) Isoform-specific expression patterns of NDRG2 in the prostate tissue cohort (n=12).

FIG. 23 shows DNA methylation EpiTYPER analysis of WFDC2 promoter in prostate cancer.

### DEFINITIONS

To facilitate an understanding of the present invention, a number of terms and phrases are defined below:

As used herein, the terms “detect”, “detecting” or “detection” may describe either the general act of discovering or discerning or the specific observation of a detectably labeled composition.

As used herein, the term “subject” refers to any organisms that are screened using the diagnostic methods described herein. Such organisms preferably include, but are not limited to, mammals (e.g., murines, simians, equines, bovines, porcines, canines, felines, and the like), and most preferably includes humans.

The term “diagnosed,” as used herein, refers to the recognition of a disease by its signs and symptoms, or genetic analysis, pathological analysis, histological analysis, and the like.

A “subject suspected of having cancer” encompasses an individual who has received an initial diagnosis (e.g., a CT scan showing a mass or increased PSA level) but for whom the stage of cancer or presence or absence of methylated genes indicative of cancer is not known. The term further includes people who once had cancer (e.g., an individual in remission). In some embodiments, “subjects” are control subjects that are suspected of having cancer or diagnosed with cancer.

As used herein, the term “characterizing cancer in a subject” refers to the identification of one or more properties of a cancer sample in a subject, including but not limited to, the presence of benign, pre-cancerous or cancerous tissue, the stage of the cancer, and the subject's prognosis. Cancers may be characterized by the identification of the expression of one or more cancer marker genes, including but not limited to, the methylated genes or promoters disclosed herein.

As used herein, the term “characterizing prostate tissue in a subject” refers to the identification of one or more properties of a prostate tissue sample (e.g., including but not limited to, the presence of cancerous tissue, the presence or absence of methylated promoters, the presence of pre-cancerous tissue that is likely to become cancerous, and the presence of cancerous tissue that is likely to metastasize). In some embodiments, tissues are characterized by the identification of the expression of one or more cancer marker genes, including but not limited to, the cancer markers disclosed herein.

As used herein, the term “stage of cancer” refers to a qualitative or quantitative assessment of the level of advancement of a cancer. Criteria used to determine the stage of a cancer include, but are not limited to, the size of the tumor and the extent of metastases (e.g., localized or distant).

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragments are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under ‘medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Such examples are not however to be construed as limiting the sample types applicable to the present invention.

### DETAILED DESCRIPTION OF THE INVENTION

The present invention relates to compositions and methods for cancer diagnosis, research and therapy, including but not limited to, cancer markers. In particular, the present invention relates to methylation levels of genes (e.g., in CGI islands of the promoter regions) as diagnostic markers and clinical targets for prostate cancer.

Beginning with precursor lesions, aberrant DNA methylation marks the entire spectrum of prostate cancer progression. Experiments conducted during the course of development of embodiments of the present invention mapped the global DNA methylation patterns in prostate tissues and cell lines using Methylplex-Next Generation Sequencing (M-NGS). Hidden Markov Model based next generation sequence analysis identified 68,000 methylated regions per sample. While global CpG Island (CGI) methylation was not differential between benign adjacent and cancer samples, overall promoter CGI methylation significantly increased from ˜12.6% in benign samples to 19.3% and 21.8% in localized and metastatic cancer tissues respectively (p-value <2×10⁻¹⁶). Distinct patterns of promoter methylation were identified around transcription start sites, where methylation occurred not only on the CGIs, but also on flanking regions and CGI sparse promoters. Among the 6,691 methylated promoters in prostate tissues, 2481 differentially methylated regions (DMRs) are cancer-specific, including numerous novel DMRs. One cancer-specific DMR was identified in the WFDC2 promoter that showed frequent methylation in cancer (17/22 tissues, 6/6 cell lines), but not in the benign tissues (0/10) and normal PrEC cells. Integration of LNCaP DNA methylation and H3K4me3 data indicated an epigenetic mechanism for alternate transcription start site utilization and these modifications segregated into distinct regions when present on the same promoter. Differences in repeat element methylation, particularly LINE-1, were observed between ERG gene fusion positive and negative cancers. This comprehensive methylome map furthers an understanding of epigenetic regulation in prostate cancer progression.

Accordingly, embodiments of the present invention provide compositions, kits, and methods useful in the detection and screening of prostate cancer. Experiments conducted during the course of development of embodiments of the present invention identified aberent methylation status of certain genes in prostate cancer. Some embodiments of the present invention provide compositions and methods for detecting such aberently methylated genes. Identification of aberently methylated genes finds use in screening, diagnostic and research uses.

### I. Diagnostic and Screening Methods

As described above, embodiments of the present invention provide diagnostic and screening methods that utilize the detection of aberent methylation of genes or promoters (e.g., including, but not limited to, those listed in Table 4 (e.g., WFDC2, MAGI2, MEIS2, NTN4, GPRC5B, C9orf125, FGFR2, AOX1, VAMP5, C14orf159, PPP1R3C, S100A16 and AMT)). Exemplary, non-limiting methods are described below. In some embodiments, methylation is increased in one or more of the described genes in patients with cancer. For example, in some embodiments, methylation of genes is increased relative to a control sample from a subject that does not have cancer (e.g., a population average of samples, a control sample, a prior sample from the same patient, etc.).

Any patient sample suspected of containing the aberrantly methylated genes or promoters may be tested according to methods of embodiments of the present invention. By way of non-limiting examples, the sample may be tissue (e.g., a prostate biopsy sample or a tissue sample obtained by prostatectomy), blood, urine, semen, prostatic secretions or a fraction thereof (e.g., plasma, serum, urine supernatant, urine cell pellet or prostate cells). A urine sample is preferably collected immediately following an attentive digital rectal examination (DRE), which causes prostate cells from the prostate gland to shed into the urinary tract.

In some embodiments, the patient sample is subjected to preliminary processing designed to isolate or enrich the sample for the aberently methylated genes or promoters or cells that contain the aberently methylated genes or promoters. A variety of techniques known to those of ordinary skill in the art may be used for this purpose, including but not limited to: centrifugation; immunocapture; cell lysis; and, nucleic acid target capture (See, e.g., EP Pat. No. 1 409 727, herein incorporated by reference in its entirety).

The methylation status of the cancer markers may be detected along with other markers in a multiplex or panel format. Markers are selected for their predictive value alone or in combination with the gene fusions. Exemplary prostate cancer markers include, but are not limited to: AMACR/P504S (U.S. Pat. No. 6,262,245); PCA3 (U.S. Pat. No. 7,008,765); PCGEM1 (U.S. Pat. No. 6,828,429); prostein/P501S, P503S, P504S, P509S, P510S, prostase/P703P, P710P (U.S. Publication No. 20030185830); RAS/KRAS (Bos, Cancer Res. 49:4682-89 (1989); Kranenburg, Biochimica et Biophysica Acta 1756:81-82 (2005)); and, those disclosed in U.S. Pat. Nos. 5,854,206 and 6,034,218, 7,718,369, 7,229,774, each of which is herein incorporated by reference in its entirety. Markers for other cancers, diseases, infections, and metabolic conditions are also contemplated for inclusion in a multiplex or panel format.

The methylation levels of non-amplified or amplified nucleic acids can be detected by any conventional means. For example, in some embodiments, Methylplex-Next Generation Sequencing (M-NGS) methodology is utilized (See e.g., experimental section below). In other embodiments, the methods described in U.S. Pat. Nos. 7,611,869, 7,553,627, 7,399,614, and/or 7,794,939, each of which is herein incorporated by reference in its entirety, are utilized. Additional detection methods include, but are not limited to, bisulfate modification followed by any number of detection methods (e.g., probe binding, sequencing, amplification, mass spectrometry, antibody binding, etc.) methylation-sensitive restriction enzymes and physical separation by methylated DNA-binding proteins or antibodies against methylated DNA (See e.g., Levenson, Expert Rev Mol Diagn. 2010 May; 10(4): 481-488; herein incorporated by reference in its entirety).

In some embodiments, a computer-based analysis program is used to translate the raw data generated by the detection assay (e.g., the presence, absence, or amount of methylation of a given marker or markers) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., methylation ata), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., presence or absence of aberrant methylation) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease or as a companion diagnostic to determine a treatment course of action.

Compositions for use in the diagnostic methods described herein include, but are not limited to, probes, amplification oligonucleotides, detection reagents, controls and the like. In some embodiments, reagents are provided in the form of an array.

### II. Drug Screening Applications

In some embodiments, the present invention provides drug screening assays (e.g., to screen for anticancer drugs). The screening methods of the present invention utilize genes with aberrant methylation. For example, in some embodiments, the present invention provides methods of screening for compounds that alter (e.g., decrease) the methylation of such genes (e.g., in the promoter region) or the number of cells containing aberrant methylation. The compounds or agents may interfere with pathways that are upstream or downstream of the biological activity of aberently methylated DNAs. In some embodiments, candidate compounds are antisense or interfering RNA agents (e.g., oligonucleotides) directed against aberently methylated DNAs. In other embodiments, candidate compounds are antibodies or small molecules that specifically bind to aberently methylated DNA regulator or expression product to inhibit its biological function.

In one screening method, candidate compounds are evaluated for their ability to alter expression of aberently methylated DNAs by contacting a compound with a cell expressing a aberently methylated DNAs and then assaying for the effect of the candidate compounds on expression. In some embodiments, the effect of candidate compounds on expression of aberently methylated DNAs is assayed for by detecting the level of aberently methylated DNA expressed by the cell. Expression of aberently methylated DNAs can be detected by any suitable method (e.g., those described herein).

### EXPERIMENTAL

The following examples are provided in order to demonstrate and further illustrate certain preferred embodiments and aspects of the present invention and are not to be construed as limiting the scope thereof.

### Example 1

### Methods

Reagents, Cell Lines and Prostate Tissue Samples

Human primary prostate epithelial cells were purchased from Lonza (Mapleton Ill.), and the prostate cancer cell line LNCaP was obtained from ATCC (Manassas, Va.). The PrEC and LNCaP cells were grown in PrEGM media (Lonza, Mapleton Ill.) and RPMI 1640 containing 10% FBS (Life Technologies, Carlsbad, Calif.), respectively. Human prostate tissue samples were obtained from the University of Michigan SPORE program (Table 3). All samples were collected with informed consent of the patients and prior institutional review board approval. CpG island microarrays were purchased from Agilent Technologies, Santa Clara, Calif. Genomic DNA was isolated from cultured cells and tissue using DNeasy Blood and tissue kit (Qiagen, Valencia, Calif.) according to manufacturer's instructions. 5-Aza-2′-deoxycytidine (5-Aza) was purchased from Sigma-Aldrich (St. Louis, Mo.) and used at 6 μM final concentration dissolved in DMSO.

M-NGS Library Generation

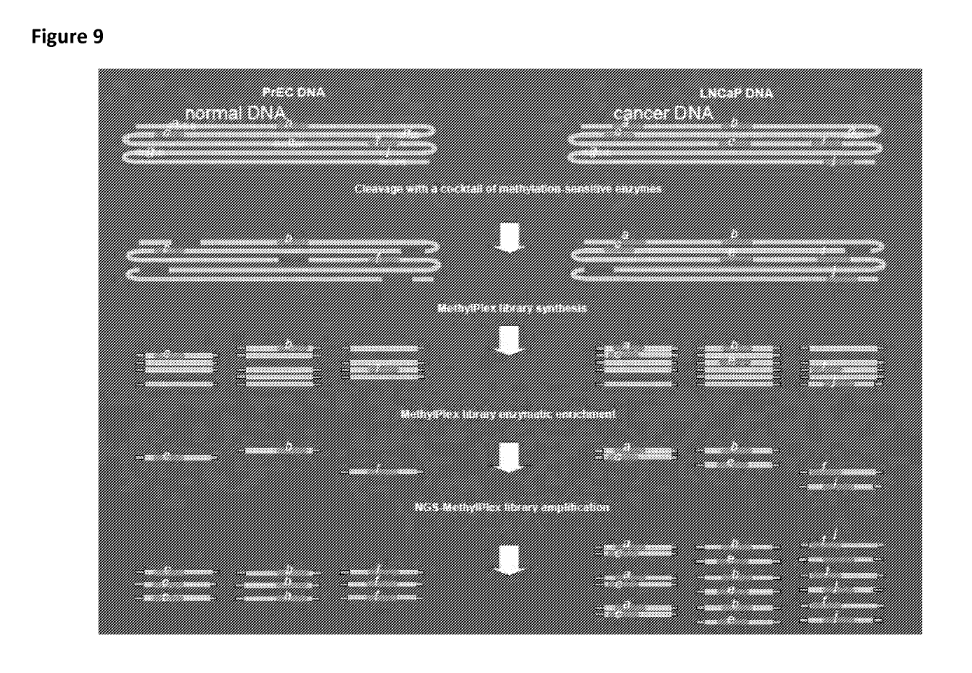

Methylplex library synthesis and GC-enrichment was obtained through a commercial service at Rubicon Genomics Inc. Ann Arbor, Mich. (FIG. 9). Briefly, fifty nanograms of gDNA from tissues or cells were digested with methylation sensitive restriction enzymes 1 and 2 (MSRE1 and MSRE2, Rubicon Genomics, Ann Arbor, Mich.) in a 100 μls reaction volume at 37° C. for 12 hours followed by 60° C. incubation for 2 hours. The samples were precipitated with two volumes of ethanol in presence of sodium acetate pH 5.2 and pellet paint (VWR, Radnor, Pa.). DNA pellets were washed with 70% ethanol, air dried and suspended in 20 μls of TE buffer pH8.0. To prepare MethylPlex libraries, ten microliters of the samples from the previous step were denatured at 95° C. for 4 minutes, cooled to 4° C., and mixed with 4 μls of library synthesis mix (Rubicon Genomics, Ann Arbor, Mich.). The tubes were incubated at 95° C. for 2 minutes, and returned to 4° C. before adding 1 μl of library synthesis enzyme (Rubicon Genomics, Ann Arbor, Mich.). The reaction was carried in a thermocycler under the following conditions, 16° C. for 20 minutes, 24° C. for 20 minutes, 37° C. for 20 minutes, 75° C. for 10 minutes and returned to 4° C. Subsequently fifteen microliters of the MethylPlex library was amplified in a BioRad iCycler real time PCR machine after mixing with 60 μls of library amplification mix (Rubicon Genomics, Ann Arbor, Mich.), under the following cycle conditions, 95° C. for 2 minutes (1 cycle), followed by 9 to 13 cycles of 96° C. for 20 seconds, 65° C. for 2 minutes and 75° C. for 1 minute. The amplified DNA was purified using QIAquick PCR purification kit (Qiagen, Valencia, Calif.), eluted in 50 μl volume and subjected to GC enrichment following the manufacturer's protocol (Rubicon Genomics Inc, Ann Arbor, Mich.). The GC enriched DNA was purified using DNA Clean and Concentrator kit (Zymo Research, Orange, Calif.) and eluted in 35 uls of Tris-EDTA buffer, pH8.0. One and five micrograms of the purified products from each cell line were directly incorporated into the genomic DNA sequencing sample preparation kit procedure from Illumina (San Diego, Calif.) at the end repair step, skipping the nebulization process. An adenine base was then added to the purified end repaired products using Klenow exo (3′ to 5′ exo minus) enzyme. The reaction product was purified, ligated to Illumina adaptors with DNA ligase and resolved on an agarose gel. For LnCaP and PrEC libraries, gel pieces were excised at 200 and 400 base pair positions and the DNA was extracted using a gel extraction kit (Qiagen, Valencia, Calif.). Subsequently for all tissue samples 350-450 bp gel cut was utilized. One microliter of this eluate was used as a template in a PCR amplification reaction with Phusion DNA polymerase (Finnzymes, INC., Woburn, Mass.) to enrich for the adapter modified DNA fragments. The PCR product was purified and analyzed by Bioanalyzer (Agilent Technologies, San Diego, Calif.) before using it for flow cell generation, where 10 nM of library was used to prepare flowcells with approximately 30,000 clusters per lane. The raw sequencing image data were analyzed by the Illumina analysis pipeline, aligned to the unmasked human reference genome (NCBI v36, hg18) using the ELAND software (Illumina) to generate sequence reads of 25-32 bps. Additional information on sequencing runs for all cells and tissue sample runs can be found in Table 3. The M-NGS data has been deposited under accession number GSE27619 in the GEO database.

Total RNA Isolation and Quantitative Real Time PCR (QPCR)

Total RNA was isolated from cells using RNeasy mini kit (Qiagen, Valencia, Calif.) according to manufacturer's instructions. A DNAseI treatment step was included during the total RNA isolation procedure to remove genomic DNA from the samples. One microgram of total RNA was used in cDNA synthesis using Superscript III reverse transcriptase (Invitrogen, Carlsbad, Calif.). Quantitative real time PCR (QPCR) was performed on prostate cell line cDNA samples using SYBR Green Mastermix (Applied Biosystems) on an Applied Biosystems 7900 Real Time PCR system as described (Tomlins et al. 2007, Nat Genet 39: 41-51). All oligonucleotide primers were synthesized by Integrated DNA Technologies and are listed in Table 2. GAPDH primers sequences were as described (Vandesompele et al. 2002, Genome Biol 3: RESEARCH0034). The amount of target transcript and GAPDH in each sample was normalized by standard ddCt methodology, and then to the reference PrEC or DMSO-treated LNCaP samples accordingly.

CpG Island Annotation

The genomic co-ordinates for human CGIs are downloaded from NCBI at the NIH. Only islands annotated as strict CpGs were used in this study.

RNA Seq Library Preparation

Poly-A RNA from LNCaP and PrEC cells (200 ng) was isolated from total RNA using SeraMag Magnetic Oligo(dT) Beads (Thermo Fisher Scientific, Waltham, Mass.). RNA was fragmented at 70° C. for 5 min in a fragmentation buffer (Ambion, Austin, Tex.), and converted to first-strand cDNA using SuperscriptII (Life Technologies, Carlsbad, Calif., Carlsbad, Calif.). Second-strand cDNA synthesis was performed with Escherichia coli DNA pol I (Life Technologies, Carlsbad, Calif.). The double-stranded cDNA library was further processed following Illumina Genomic DNA sample preparation protocol which involved end repair using T4 DNA polymerase, Klenow DNA polymerase and T4 Polynucleotide kinase followed by a single ‘A’ base addition using Klenow 3′ to 5′ exo⁻ polymerase. Illumina's adaptor oligo was ligated using T4 DNA ligase. The adaptor-ligated library was size selected by separating on a 4% agarose gel and cutting out the library smear at 200 base pairs. The library was PCR amplified by Phusion polymerase (Finnzymes, Woburn, Mass.) and purified by PCR purification kit (Qiagen, Valencia, Calif.). The library was quantified with Bio-Analyzer (Agilent Technologies, Santa Clara, Calif.) and 10 nM of each library was used to prepare flowcells with approximately 30,000 clusters per lane. The GEO accession number for LNCaP and PrEC RNA-Seq libraries is GSE25183

Statistical Analysis

HMM Analysis of M-NGS Data

Hidden Markov Model (HMM) based next generation sequencing analysis is conducted in a two-step process that takes in raw reads and outputs refined boundaries of enriched chromosomal regions (Qin et al. 2010, BMC Bioinformatics 11: 369). The first step includes the formation of hypothetical DNA fragments (HDFs) from uniquely mapped reads, where the coverage of HDFs is determined by the specified DNA fragment size and overlapped HDFs are merged to represent one consecutive genomic region. The second step is designed to refine the boundaries of enriched region using HMM with bin size of 25 bp (by default). Under null hypothesis, raw reads are assumed to land on the genome following a Poisson distribution with the background rate of r⁰, and enriched regions are expected to have more HDFs with statistical significance. The rate of the Poisson distributions in a given sample is assumed to be r¹, and the transition probabilities are estimated empirically, based on inferred enriched regions defined in the first step. The output from HMM is selected based on the posterior probability of being in the enriched regions and then further filtered using maximum read counts. The threshold for maximum read counts is determined from Bonferroni corrected p-value of 0.001 calculated using a Poisson distribution with background rate r⁰. The output is provided in BED format as well as Wiggle format for UCSC genome browser visualization. The output file annotation field contains information such as enriched genomic position and length, max height, GC content, repeated sequencing genomic position and length, mean and standard deviation of conservative scores for enriched region, relationship with nearest genes including whether the enriched region is located within the gene or between genes, gene name, GB accession number, strand and distance to gene transcription start site.

Calculating Gene Expression from RNA-Seq Data

Gene expression levels of passing filter reads from RNA-Seq data that mapped by ELAND to exons (March 2006 assembly of UCSC KnownGene) in LNCaP and PrEC cell lines are quantified as described (Maher et al. Nature 458: 97-101 2009).

One-Class SAM Analysis

Significance analysis of microarray (SAM) (Tusher et al. Proc Natl Acad Sci USA 98: 5116-5121 2001) was performed on the gene expression dataset obtained from 5-Aza and DMSO-treated LNCaP cells by selecting genes that were methylated in LNCaP. From 1,171 methylated genes from LNCaP M-NGS (Table 1), a total of 973 genes were mapped to Agilent expression profiling data. One-class SAM analysis was done using default settings and significant genes were calculated with a false discovery rate (FDR) of 0.05.

Gene Set Enrichment Analysis

Gene Set Enrichment Analysis (GSEA) is a computational method that assesses whether a defined set of genes shows statistically significant, concordant differences between any two given conditions. The fold change between the raw counts from RNA-seq NGS data on LNCaP and PrEC (representing 24,167 unique genes) was calculated and genes were ranked by the order of expression in LNCaP. This list was uploaded as a pre-ranked gene list to GSEA v2.04 (Broad Institute, Cambridge, Mass.), and using respective gene lists of methylated targets in LNCaP and PrEC cell lines, GSEA was performed using a weighted enrichment statistic and default normalization mode. Similarly, the fold change between the average expression value from normal/benign (n=4) and cancer samples (n=9) profiled on Agilent Human GE 44K microarray was calculated and pre-ranked (representing 27,928 unique probes). This list was uploaded to GSEA, and enrichment analysis was performed using methylation target gene lists (the methylation present in promoters with CGIs and without CGIs, and in gene body) in tumor samples.

Oncomine Meta-Analysis

A complete description of meta-analysis performed in Oncomine is available (Rhodes et al. Neoplasia 9: 166-180 2007). In brief, a genelist of interest is uploaded to the Oncomine database, and the built-in meta-analysis tool rank-orders the genelist by the p-value, which is determined by Student's t-test for comparisons made within each available dataset (for example Cancer vs. Normal). The ranked genes were visualized with pink and green shades (top ranked ones with darker shades, pink for over-expression and green for repression) in heatmap format, with each row representing genes and each column representing the dataset. Final order of the genes is determined by averaging ranks across the datasets.

Molecular Concepts Map Analysis

A complete description of the methods used to identify biological concept signatures in Molecular Concepts Map (MCM) is available (Rhodes et al. Neoplasia 9: 443-454 2007). In addition to over 15,000 biological concepts from Oncomine, which include manual curation of the literature, target gene sets from genome-scale regulatory motif analyses, and reference gene sets from several gene and protein annotation databases, a gene list from differentially methylated regions identified from an independent Differential Methylation Hybridization profiling (concept named “DMH-Tissue Methylated in PCa”), as well as known methylated genes in cancers provided from Pubmeth database were uploaded. In brief, MCM analysis uses Fisher's exact test to find various significantly enriched concepts in an uploaded gene list and provides visual interaction networks.

Repeat Element Methylation Analysis

The list of repeat elements predicted by RepeatMasker (RepeatMasker Open-3.0) program is downloaded from UCSC genome browser. The Methylplex-NGS data from localized and metastatic prostate tissue samples are divided into two groups based on their ETS gene fusion status (ETS positive and ETS negative). The samples in each group were pooled together for HMM analysis and the regions identified were mapped to repeat element location.

ChIP-Sequencing

LNCaP cells ChIP-Seq data obtained using H3K4me3 antibody (Abeam) and PanH3 (Abeam) was reported previously by Yu et al., and the GEO accession number for the dataset is GSE14097 (Yu et al. Cancer Cell 17: 443-454 2010). ChIP samples were prepared for sequencing using the Genomic DNA sample prep kit (Illumina) following manufacturer's protocols. To facilitate ChIP-Seq data analysis, a Hidden Markov Model (HMM)-based enriched region identifying algorithm (described under statistical analysis) was utilized.

MeDIP-Sequencing

Six micrograms of genomic DNA isolated from LNCaP cells were sonicated to ˜100-500 bp range and purified using Qiagen PCR purification kit. Using standard Illumina protocol/reagents ends were repaired, A tailed and adaptors were added to the fragmented DNA. The DNA was then heat denatured at 95° C. for 10 minutes and snap cooled on ice. The DNA was incubated with 6 μg anti-methyl cytosine antibody in IP buffer (10 mM sodium phosphate buffer containing 140 mM sodium chloride and 0.05% Triton X-100) overnight at 4° C. in a shaker. The methylated fragments were collected by incubating with 100 μls of protein A beads (Invitrogen, Carlsbad, Calif.) for 2 hour at 4° C. The beads were washed four times at 4° C. in IP buffer and resuspended in 200 μls of TE buffer containing 0.25% SDS and 5 μg proteinase K and incubated at 55° C. for two hours. The samples were purified using DNA Clean and Concentrator-5 kit (Zymo Research, Orange, Calif.) and the libraries were prepared following Illumina ChIP-Seq protocol. The library was quantified with Bio-Analyzer (Agilent Technologies, Santa Clara, Calif.) and 10 nM of each library was used to prepare flowcells with approximately 30,000 clusters per lane.

Methyl-Profiler

Methyl-Profiler™ (SABiosciences, Frederick, Md.) is a restriction enzyme digestion based novel technology for CGI methylation profiling, requiring less than 500 ng input genomic DNA (Jaspers et al. Am J Respir Cell Mol Biol 43: 368-375 2010). The samples were first digested with methylation-sensitive (Ms) and/or methylation-dependent (Md) restriction enzymes along with mock digestion according to manufacturer's instruction. PCR reactions were performed with ABI StepOne qPCR machine (Applied Biosystems, Foster City, Calif.) with RT² SYBR Green/ROX qPCR Master Mix (SABiosciences, Frederick, Md.) and primers targeting the region of interest. The PCR reactions were carried out with following conditions: 10 min at 95° C., followed by 40 cycles of 97° C. for 15″, 72° C. 1 min as described in manufacturer's protocol. Using delta-Ct values, the relative amounts of methylation are calculated using an automated Excel-based data analysis template provided by the manufacturer. The mock digested template was used for initial DNA input quantification, the Ms enzyme was used for hypermethylation quantification, and the Md enzyme was used for quantifying unmethylated DNA. A mixture of these 2 enzymes (Msd) was used to quantify the undigested amount of DNA. A methylation rate below 5% was considered not significant. While the calculated methylation percentage between 10 and 60 was considered intermediate, values above sixty are taken as heavy methylation.

Bisulfite Sequencing

Bisulfite conversion was carried out using EZ DNA methylation gold kit (Zymo Research, Orange, Calif.) according to manufacturer's instructions. Briefly 500 ng of genomic DNA from either LNCaP or PrEC cells in 20 ul volume was mixed with 130 μl of CT conversion reagent and was initially incubated at 98° C. for 10 minutes followed by incubation at 64° C. for 2.5 hours. M-biding buffer (600 μl) was added to the above reaction and DNA purified using a Zymo spin column. Sequential washes were performed with 100 μl M-Wash buffer, 200 ul M-sulphonation buffer and 200 μl of M-wash buffer was carried out before eluting the DNA in 30 μl of M-elution buffer. Purified DNA (2 μl) was used as template for PCR reactions with primers (Integrated DNA Technologies, San Diego, Calif.) and synthesized according to bisulfite converted DNA sequences for the regions of interest using the Methprimer software (Li and Dahiya, Bioinformatics 18: 1427-1431 2002). The PCR product was gel purified and cloned into pCR4 TOPO TA sequencing vector (Life Technologies, Carlsbad, Calif.). Plasmid DNA isolated from 10 colonies from each sample was sequenced by conventional Sanger Sequencing (University of Michigan DNA Sequencing Core). The “BIQ Analyzer” (Bock et al. 2005) online tool was used to calculate the methylation percentage and to generate the bar graphs.

Microarray Profiling

Expression Profiling of 5-Aza Treated LNCaP Cells

For 5-Aza stimulation experiments, LNCaP cells cultured in RPMI 1640 were treated with vehicle, dimethyl sulfoxide (DMSO) or 6 μM 5-Aza for 4 or 6 days in duplicates. Total RNA was isolated with Trizol (Life Technologies, Carlsbad, Calif.) and further purified using RNAeasy Micro Kit (Qiagen, Valencia, Calif.) according to the manufacturer's instructions. Expression profiling was performed using the Agilent 44K expression array. One microgram of total RNA was converted to cRNA and then labeled according to the manufacturer's protocol (Agilent). Hybridizations were performed for 16 h at 65° C. Scanned images from Agilent microarray scanner were analyzed and extracted using Agilent Feature Extraction Software 9.1.3.1 with linear and lowess normalization performed for each array. A total of 4 hybridizations were performed including two 4 day and two 6 day 5-Aza treated samples (Cy5) against control DMSO-treated samples (Cy3). The accession number for gene expression dataset in the GEO database is GSE27619.

Expression Profiling of Prostate Tissues

Prostate tissues characterized by M-NGS, normal/benign (n=4) and cancer (n=9), was profiled on Agilent Human GE 44K microarray as described for LNCaP cells above. Total RNA from pooled normal prostate tissues obtained from a commercial source (Clontech laboratories, Mountain View, Calif.) was used as the reference. This microarray dataset was used in GSEA analysis to study the association between DNA methylation and gene expression. The dataset has been deposited in the GEO database.

Methylplex Library Agilent CpG Array Hybridization

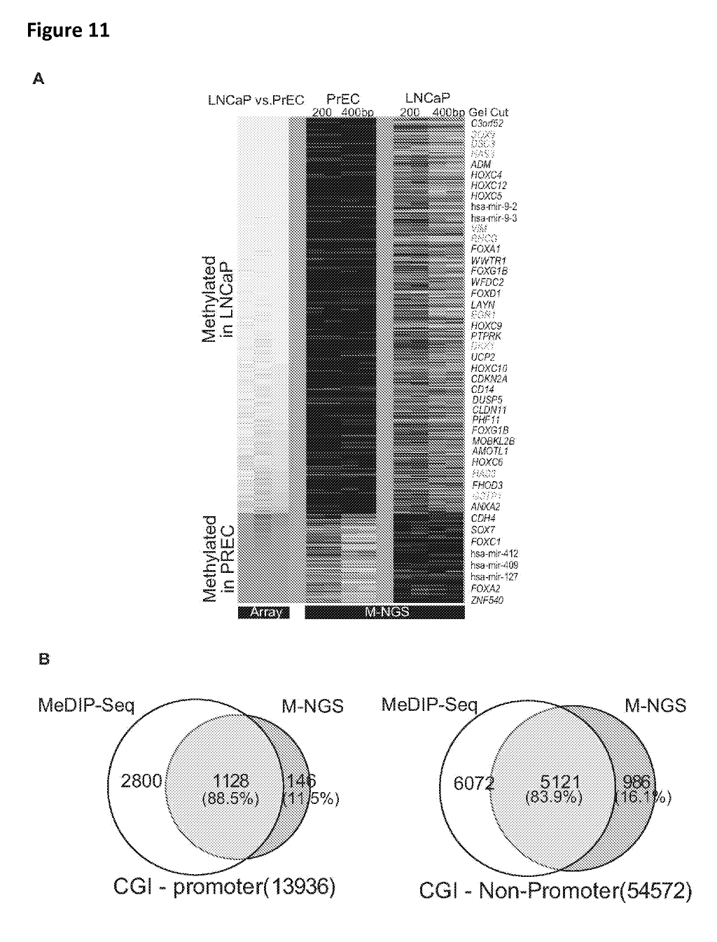

Two micrograms of the purified products from each PrEC and LNCaP MethylPlex DNA were labeled following the mammalian ChIP-on-chip protocol (Agilent Technologies, Santa Clara, Calif.) starting at the sample labeling stage which employs a random primed, Klenow-based extension protocol. The samples were hybridized to an Agilent Human CpG 244K array (Cat# G4492A, Agilent Technologies, Santa Clara, Calif.), where LNCaP sample was coupled with Cy5 and PrEC to Cy3. The slides were washed according to manufacturer's instructions. A dye-flip experiment was also performed. The scanned images were analyzed and extracted using Agilent Feature Extraction Software 9.1.3.1. Methylated regions identified by the array data was compared to M-NGS targets and their overlap is presented in FIG. 11. This dataset has been deposited in GEO under accession number GSE27619.

5′ Rapid Amplification of cDNA Ends (5′ RACE)

5′ RACE was performed as previously described (Han et al. Cancer Res 68: 7629-7637 2008). First-strand cDNA was amplified with gene-specific reverse primers RASSF1, APC, and NDRG2 (Table 2) and 5′ GeneRacer primers (Life Technologies, Carlsbad, Calif.) using Platinum Taq High Fidelity enzyme (Life Technologies, Carlsbad, Calif.) after the touchdown PCR protocol according to manufacturer's instructions. PCR amplification products were cloned into pCR4-TOPO TA vector (Life Technologies, Carlsbad, Calif.) and sequenced bidirectionally using vector primers as described (Tomlins et al. Nature 448: 595-599 2007).

Pyrosequencing

LINE-1 element methylation was estimated using PyroMark Q24 LINE-1 methylation assay (Qiagen, Valencia, Calif.) according to manufacturer's instructions. Briefly, bisulfite converted gDNA (described above), LINE-1 primers and components of Hotstart Master Mix (Qiagen, Valencia, Calif.) were employed in a PCR reaction to amplify LINE regions from the sample. The amplification was obtained from 45 cycles of 95° C. 20 seconds, 50° C. 20 seconds, 72° C. 20 seconds, after an initial denaturation/enzyme activation at 95° C. for 15 minutes, and final elongation of 72° C. for 5 minutes. The PCR products were captured on Streptavidin Sepharose beads (GE Healthcare, Piscataway, N.J.) denatured to produce single strands, washed and annealed to sequencing primer and the sequence determined using the PyroMark Q24 system (Qiagen, Valencia, Calif.). The mean methylation of three individual positions within the PCR product is considered in this assay.

### Results

Characterization of DNA Methylation by M-NGS in Prostate Cells

To perform a genome-wide analysis of DNA methylation in prostate cancer, Methylplex-Next Generation Sequencing (M-NGS) methodology, which enriches methylated DNA using restriction enzymes and requires minimal input genomic DNA (i.e., 50 nanograms) was utilized. The ability of M-NGS to identify methylated genomic regions was first evaluated in a prostate cancer cell line LNCaP and normal PrEC cells. A schematic describing sequencing library generation is provided in FIG. 9. Briefly, Methylplex libraries were constructed by digesting input genomic DNA isolated from samples with a cocktail of methylation-sensitive restriction enzymes, followed by ligation of adaptors containing universal primers sequences and PCR-based amplification. A second round of enzymatic treatment depleted non-GC rich sequences, followed by an additional amplification step to ensure enrichment of highly methylated DNA fragments. The amplification adaptors were enzymatically removed prior to NGS library preparation (FIG. 9). Methyplex libraries described above were constructed through the commercial service provided by Rubicon Genomics Inc, Ann Arbor, Mich.

For initial standardization, two different concentrations (1 and 5 μg) of each Methylplex sample from LNCaP and PrEC cells was used as input DNA to obtain single-read sequencing on the Illumina Genome Analyzer II (see Methods for protocol details). For each cell type (LNCaP and PrEC) a total of 4 sequencing libraries were prepared corresponding to 200- and 400-bp size selections of 1 μg and 5 μg of Methylplex product. An average of 5 million mappable reads was obtained for each M-NGS sample. CG dinucleotides were enriched by the Methylplex procedure up to three-fold in mapped reads from M-NGS compared to previously obtained control ChIP-Sequencing data, namely pan-histone ChIP-Seq (Yu et al. Cancer Cell 17: 443-454 2010).

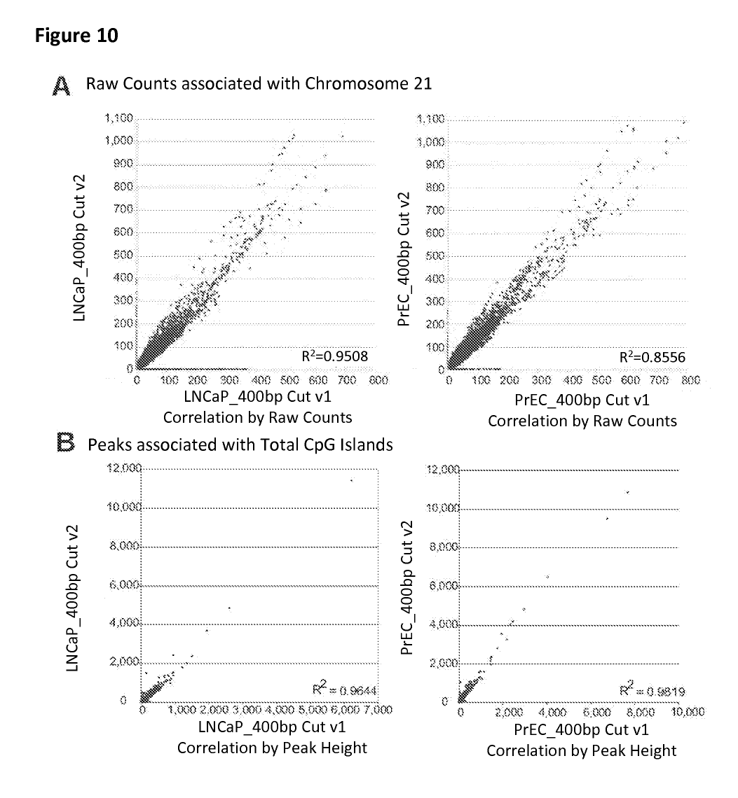

To demonstrate experimental consistency, a comparative analysis of data from 1 and 5 μg Methylplex DNA exhibited high correlation both for reads mapping to chromosome 21 and for reads mapping to all CpG islands (FIG. 10). Data from 400 bp-5 ug was most enriched for CG rich sequences and showed maximum overlap (˜70%) with methylation identified by hybridizing the Methylplex product to a CpG island array (FIG. 11A). This data was selected for further analysis.

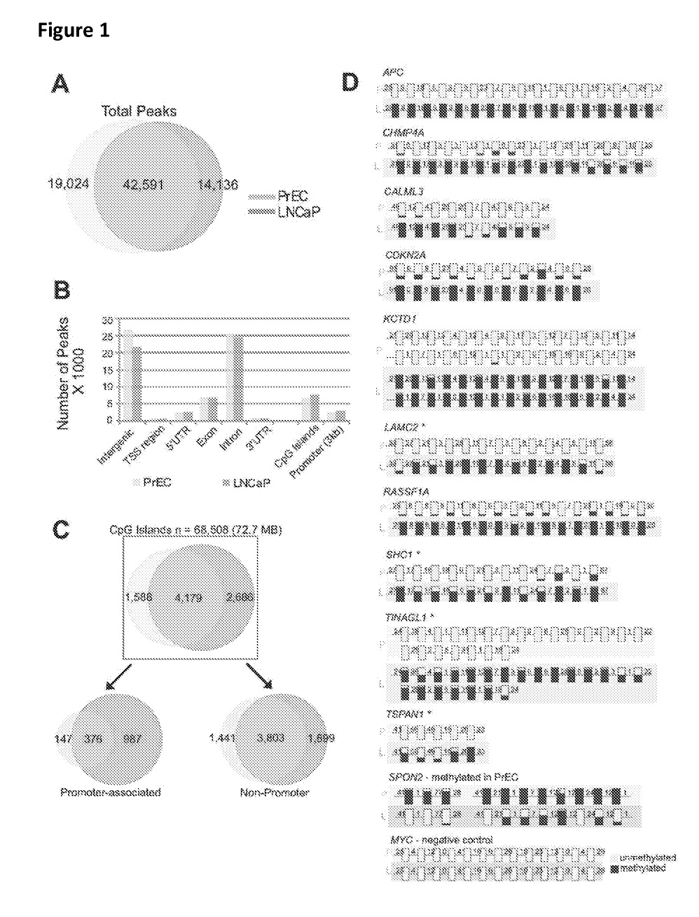

A Hidden Markov Model (HMM)-based algorithm previously used for ChIP-Seq data analysis (Qin et al. BMC Bioinformatics 11: 369 2010) was used to locate peaks from mapped reads obtained in each sequencing run. A 70% overlap in methylated genomic regions between LNCaP (56,727 regions) and PrEC cells (61,615 regions) was found (FIG. 1A). Methylation located in intergenic and intronic regions of the genomes analyzed had a similar distribution (FIG. 1B), additionally, in LNCaP cells, MeDIP-Seq, a methodology that employs 5′ methylcytosine antibody to enrich methylated regions was used to identify ˜68,000 methylated regions in this cell line, which was comparable to the M-NGS results. Moreover, there was an overall 62% concordance between all the genomic regions (data not shown) and >83% in CGIs identified by M-NGS and MeDIP-Seq, thereby validating the two methodologies (FIG. 11B).

The cancer-derived LNCaP cells displayed frequent methylation among the 56 previously reported methylated promoter regions in prostate cancer tissues (36/56 in LNCaP M-NGS and 40/56 in LNCaP MeDIP-Seq) compared to PrEC cells (7/56 in PrEC M-NGS) (Table 5). However, this difference was absent when the promoters and gene body of known imprinted genes was examined (Morison et al. Trends Genet 21: 457-465 2005) (24/29 in PrEC M-NGS, 23/29 in LNCaP M-NGS and 26/29 in LNCaP MeDIP-Seq) (Table 5).

Global Differences in CGI Methylation

Because hypermethylation in CpG rich promoters is a common feature of tumorigenesis (Issa Nat Rev Cancer 4: 988-993 2004), the extent of CpG island methylation between LNCaP and PrEC cells was compared. Of the 68,508 (72.74 MB) CpG islands identified using Takai Jones criteria (Takai and Jones Proc Natl Acad Sci USA 99: 3740-3745 2002) in the human genome, 6,865 (7.6 MB) and 5,767 (6.1 MB) CpG islands were methylated in LNCaP and PrEC respectively. Globally, a 1.7-fold increase in uniquely methylated CpG islands between LNCaP and PrEC was observed, and this ratio increased to ˜7-fold specifically in CpG islands associated within gene promoters but not among CGIs located elsewhere (FIG. 1C). In LNCaP cells, methylation in greater than 88% of CpG islands located within promoters and 83% of CGIs in non promoters detected by M-NGS was corroborated by the MeDIP-Seq data (FIG. 11 B).

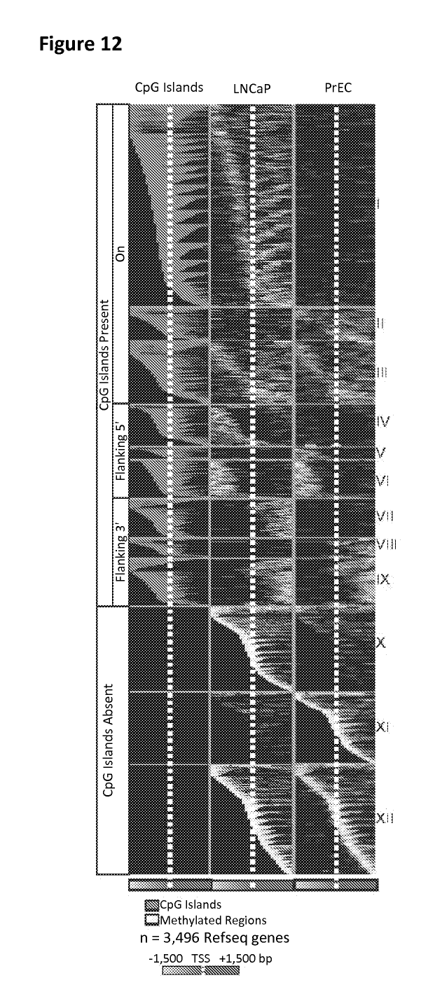

Aberrant promoter methylation is thought to contribute to tumorigenesis by repressing transcription of tumor-suppressor genes (Jones and Baylin Cell 128: 683-692 2007). Methylation on Ref-Seq gene promoters (±1,500 bps flanking transcription start site) was analyzed and 3,496 locations that were methylated in at least one sample were identified (FIG. 12). Visualization of these methylation marks in the context of promoter CGIs revealed the presence of several distinct methylation patterns on gene promoters (FIG. 12). Broadly, the promoters fell into two groups based on the presence or absence of a CpG island within this specified region. Although 35% of promoters (n=1,232) lacked CpG islands, they exhibited methylation around the transcription start site (TSS) (FIG. 12). The remaining 65% (n=2,264) had CpG islands spanning the TSS and three distinct methylation patterns were observed in this group: (1) methylation was mostly confined (39.6%, n=1,383) to the island, and interestingly with much higher frequency (greater than 6 fold difference) in LNCaP (n=952) compared to PrEC (n=147) cells (FIG. 12). (2) methylation was positioned 5′ to the CpG island (11.8%, n=412); and (3) methylation was positioned 3′ to the CpG island (13.4%, n=469). In total, methylation flanking 5′ or 3′ of promoter CpG islands accounted for 25.2% of all methylation observed (n=881). To explore the role of these methylation patterns in prostate cancer pathogenesis, 812 out of 1171 unique gene promoters to be methylated only in LNCaP were identified (Table 1) and considered for further analysis. The remaining 359 promoters were methylated in both LNCaP and PrEC cells.

Validation of DMRs

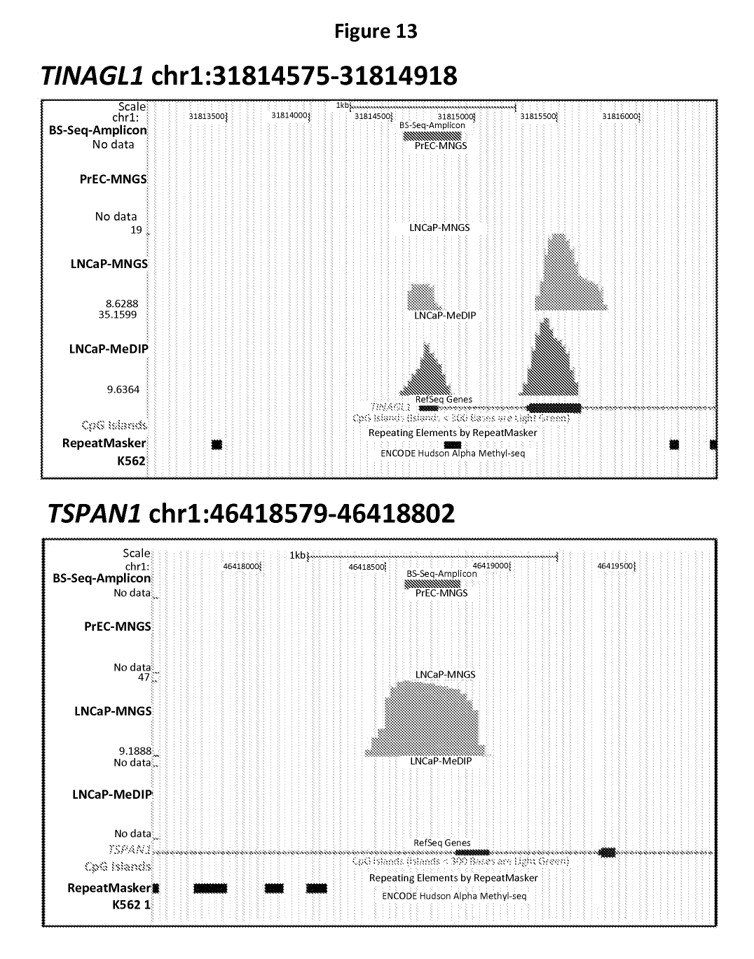

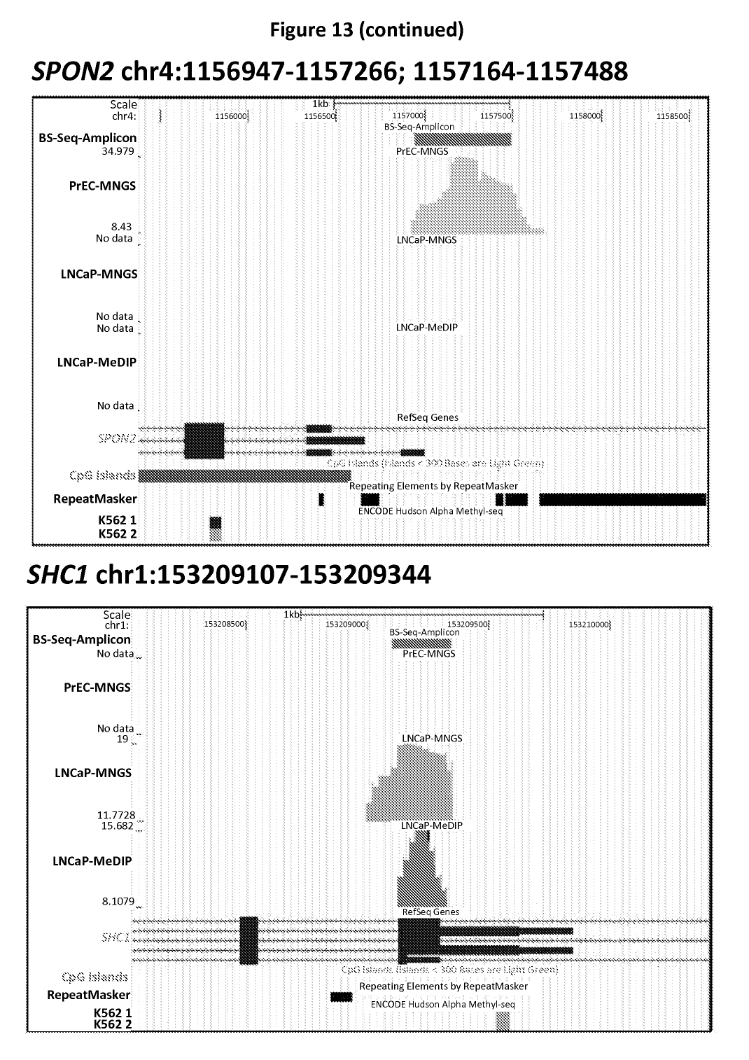

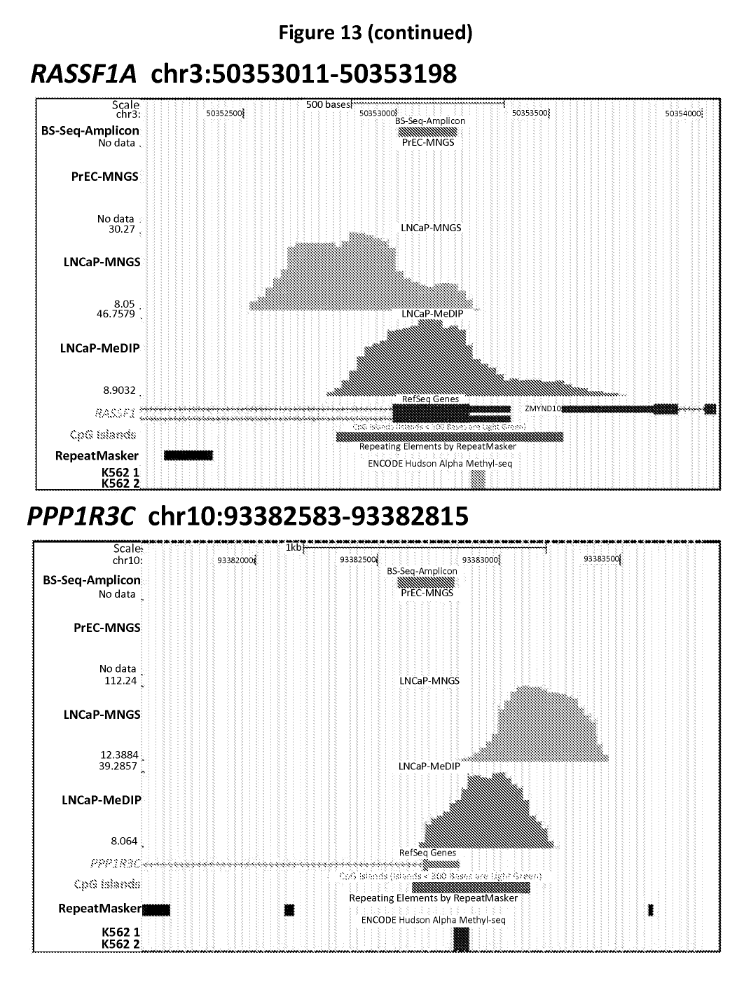

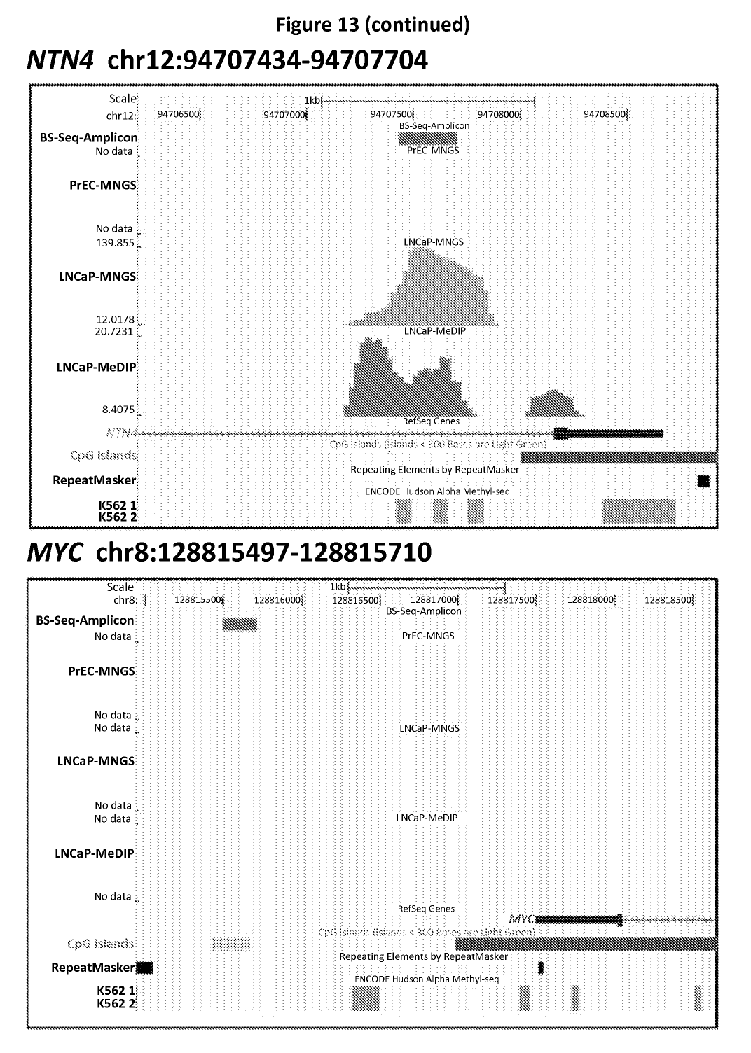

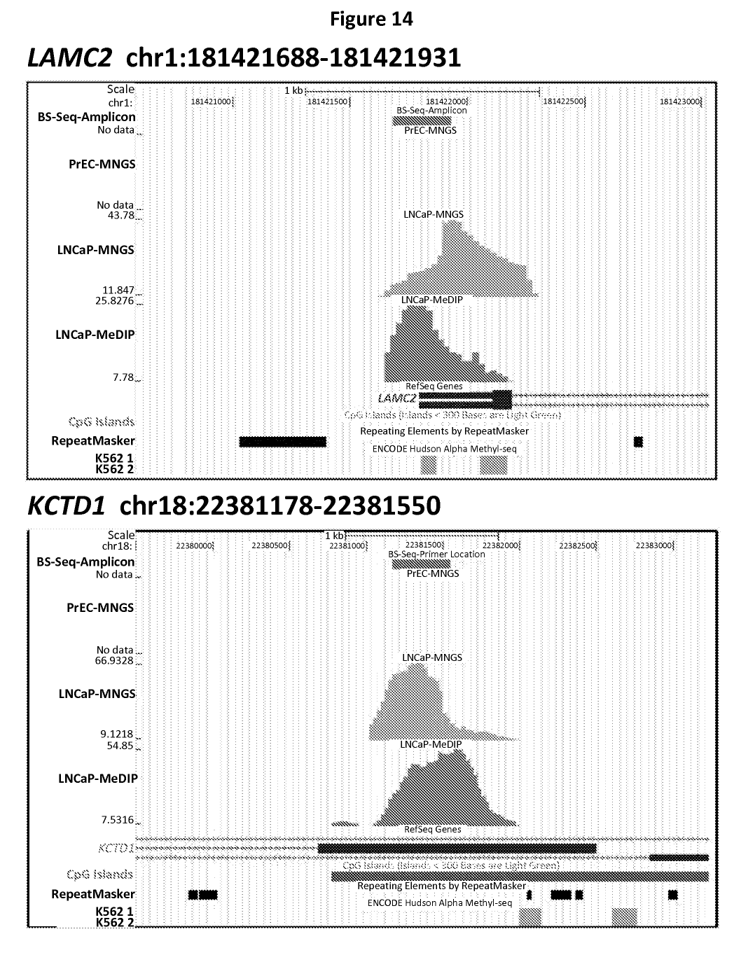

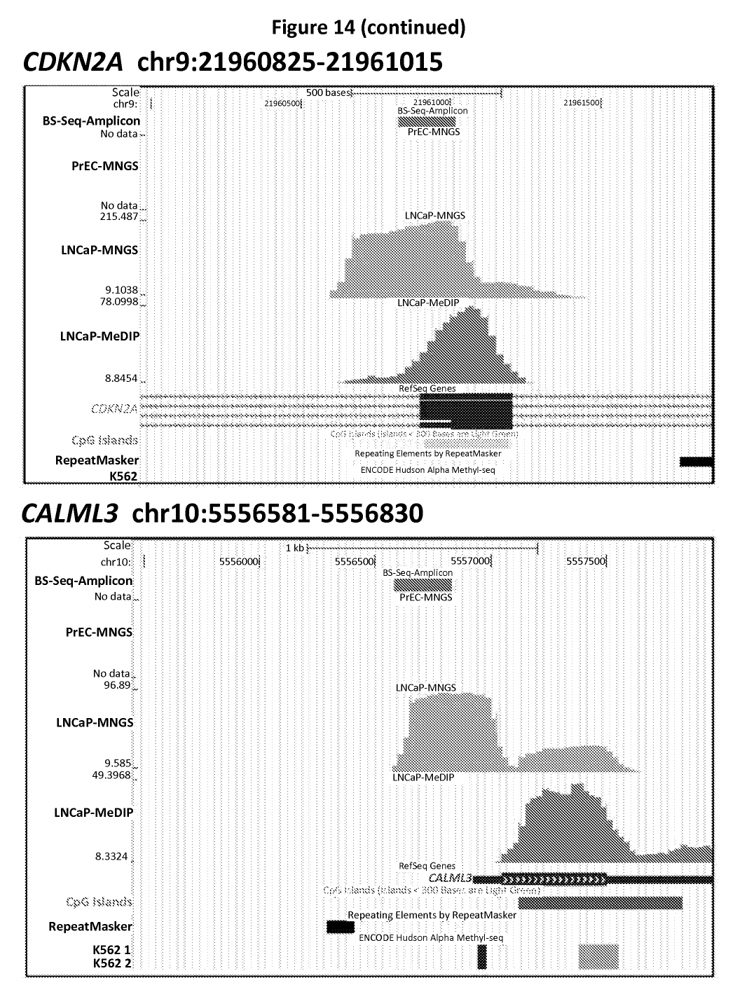

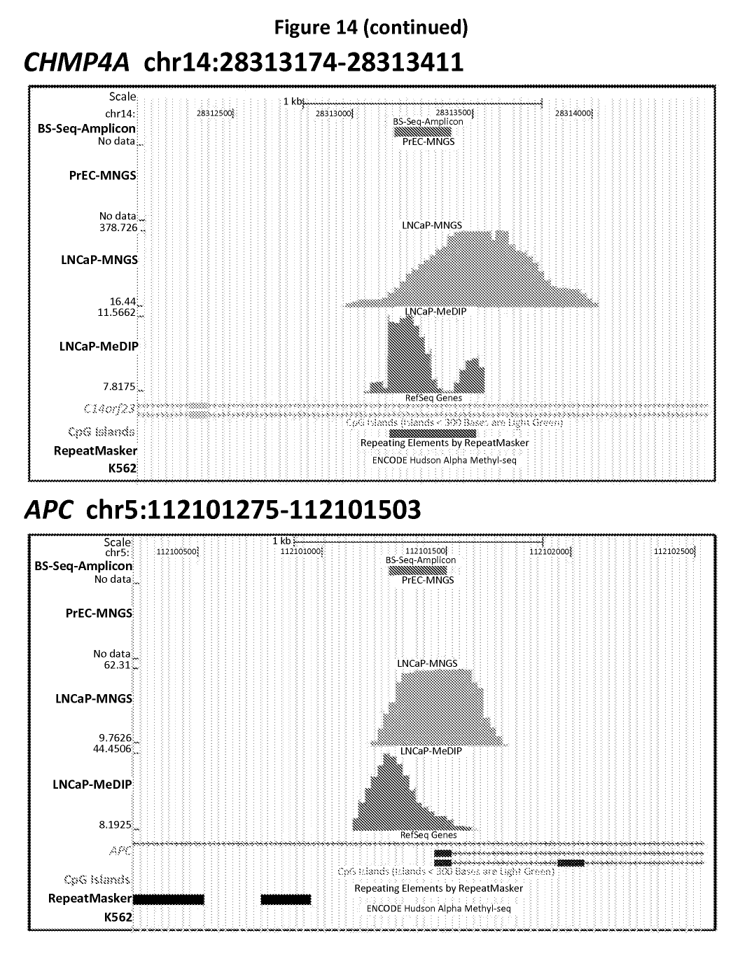

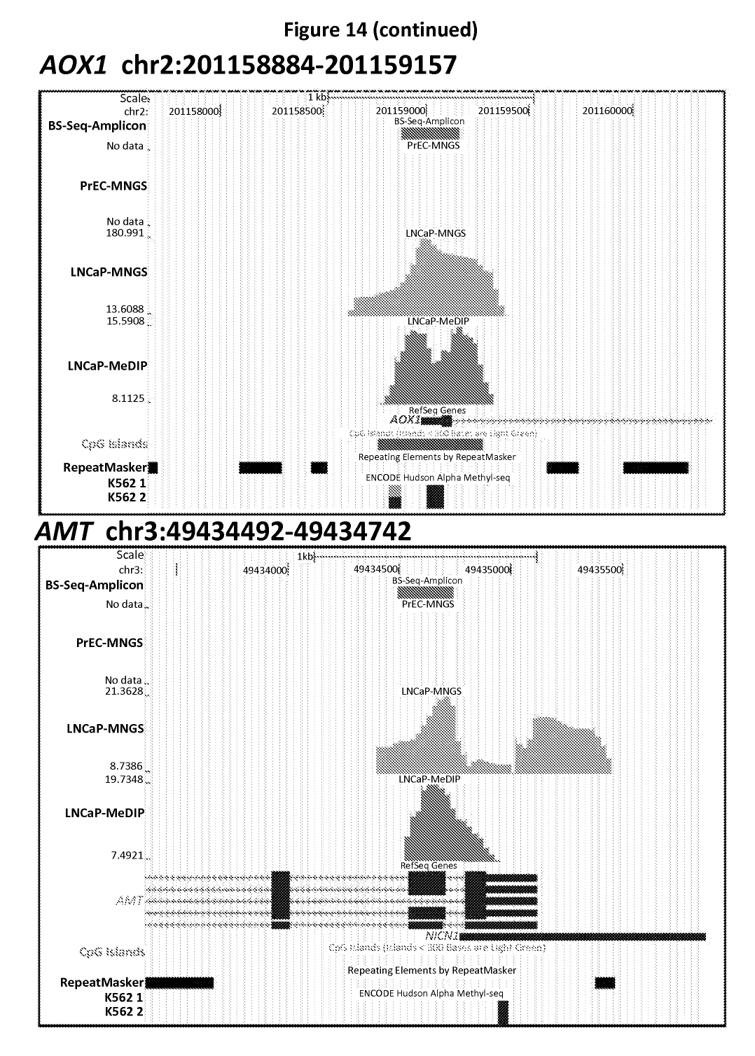

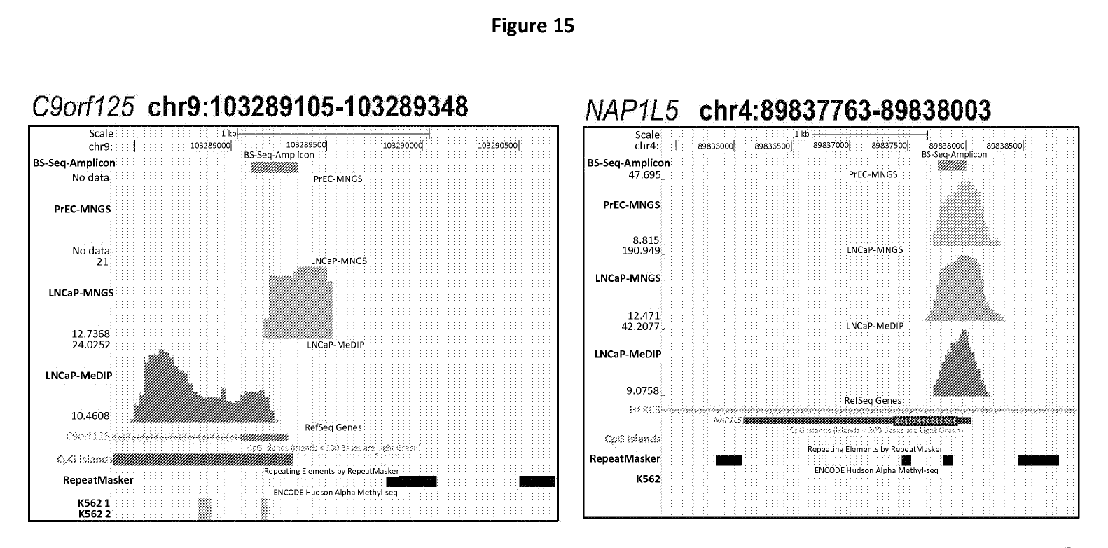

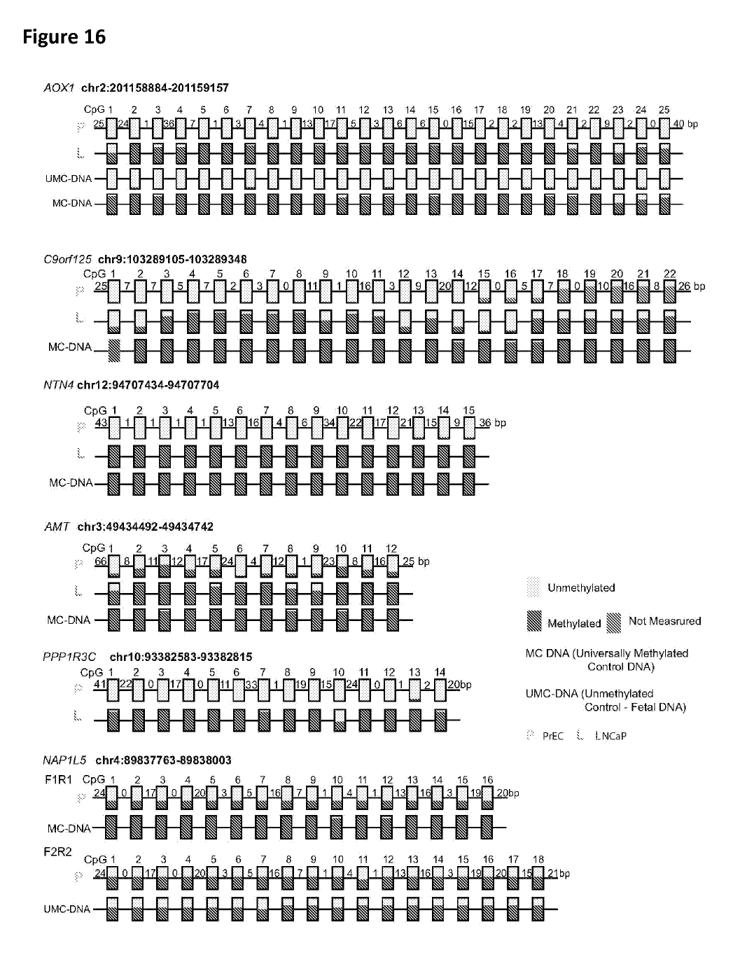

18 regions based on M-NGS data were selected and their methylation status was validated using a standard bisulfite sequencing technique in LNCaP and PrEC cells. This included fifteen DMRs in LNCaP (RASSF1, KCTD1, CHMP4A, APC, CDKN2A, SHC1, LAMC2, TSPAN1, CALML3, AOX1, AMT, C9orf125 and TINAGL1), one gene in PrEC cells (SPON2), one region methylated in both LNCaP and PrEC cells (NAP1L5) and a control MYC promoter region that was unmethylated in both cell types. The UCSC genome browser view of methylation in the two samples by M-NGS and methylation in LNCaP by MeDIP-Seq, along with gene schematic, primer sequences and bisulfite sequence amplicon locations are presented in FIGS. 12 to 15 and Table 2. Notably, the results for all 18 regions confirmed the data generated by M-NGS (FIG. 1D and FIG. 16).

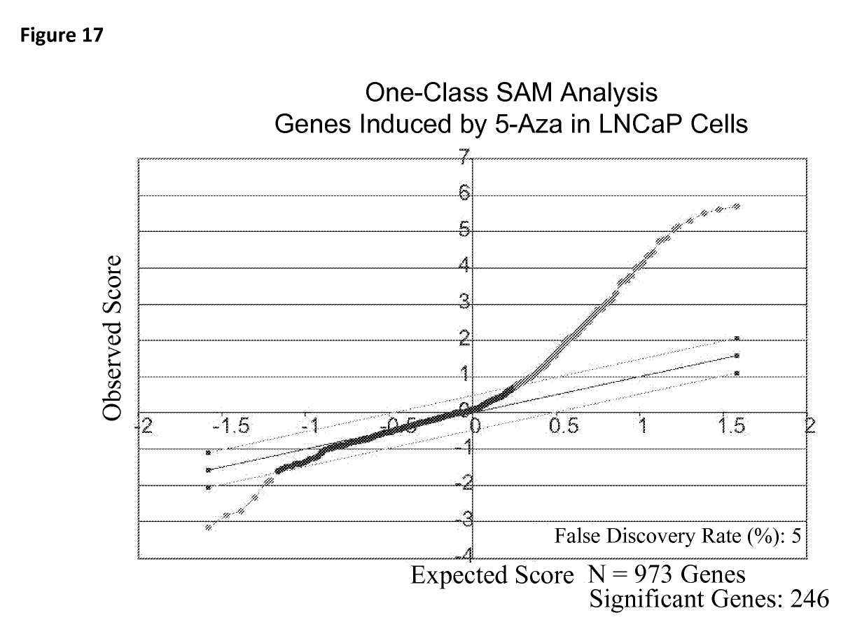

In addition, overexpression of a significant number of LNCaP methylated genes following 5-Aza treatment of cells in a functional validation strategy using gene expression arrays was observed. A total of 973 out of 1171 methylated genes in LNCaP were present in gene expression array data. Significance Analysis of Microarray (SAM) results showed upregulation of 246 out of 973 methylated genes at 5% false discovery rate (FIG. 17), supporting epigenetic regulation of these genes.

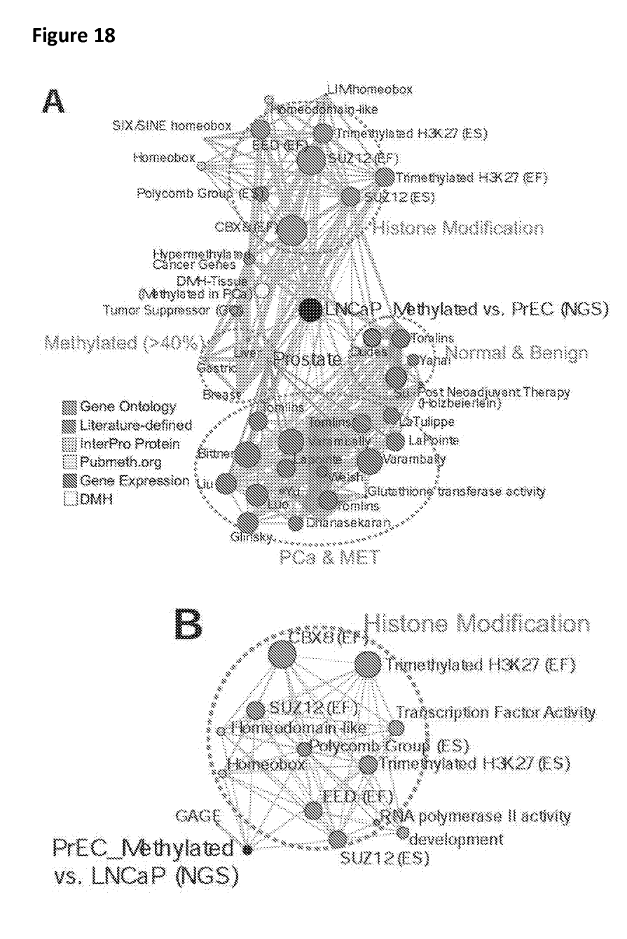

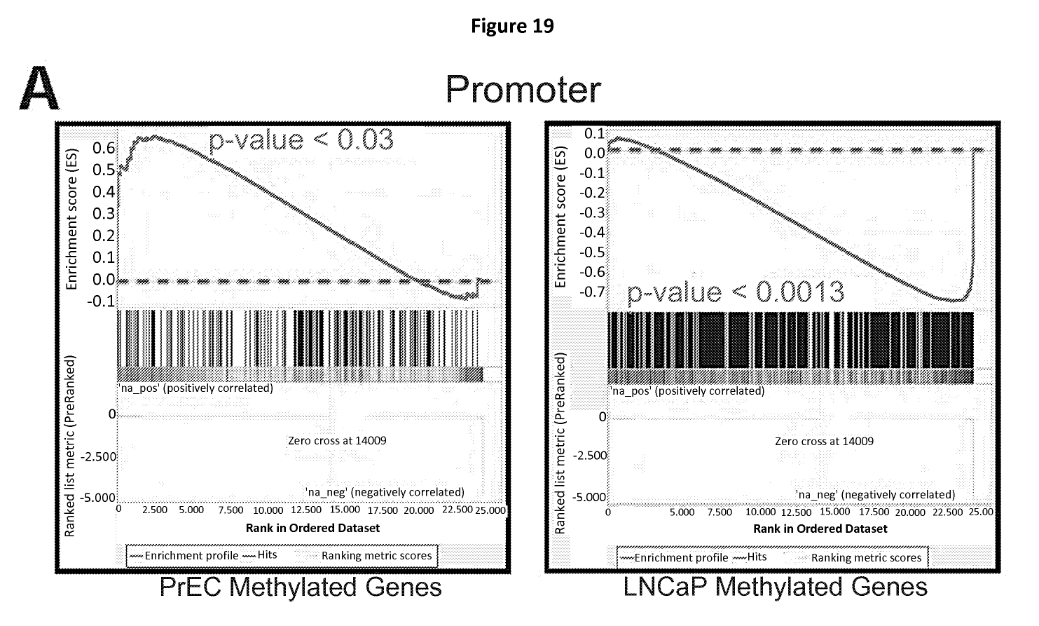

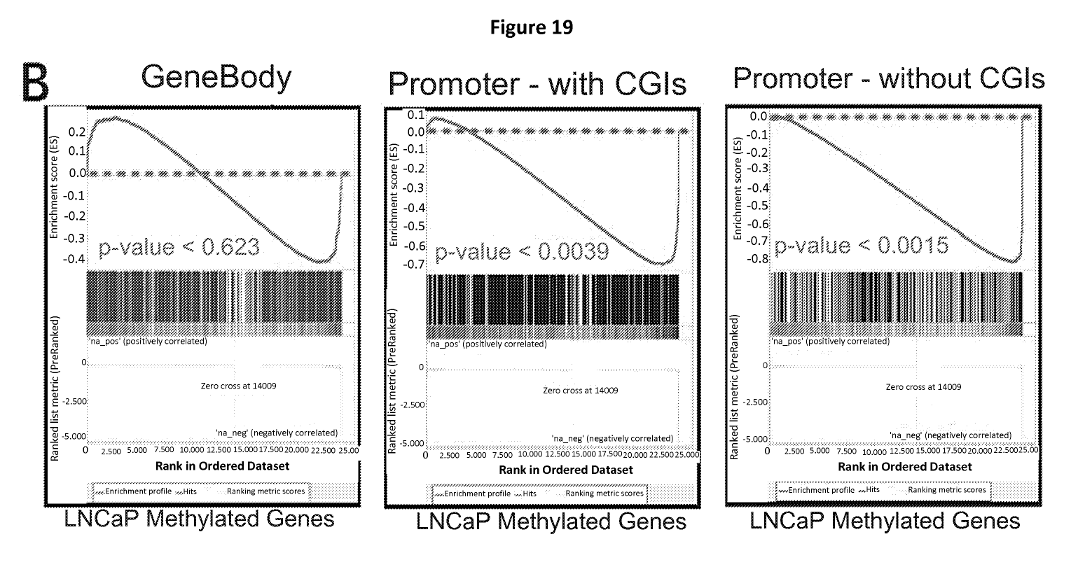

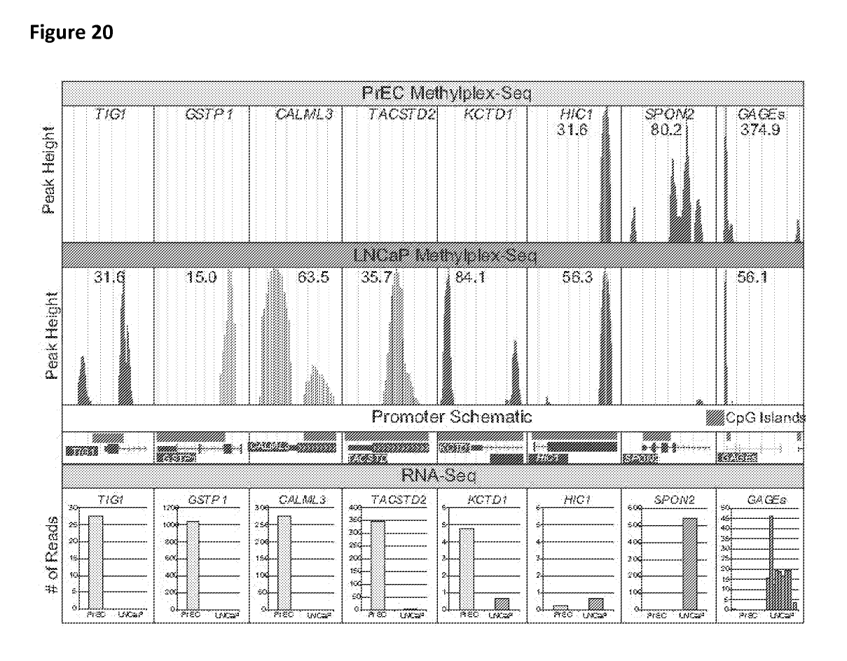

To identify molecular concepts enriched in our DMRs, the dataset was analyzed using the Molecular Concept Map (MCM) analysis derived from the Oncomine database (Rhodes et al. Neoplasia 9: 443-454 2007; Tomlins et al. Nat Genet 39: 41-51 2007). MCM analysis of 789 out of 813 genes methylated only in LNCaP that mapped to the Oncomine database (FIG. 18 and Table 1), revealed preferential enrichment with under-expressed genes signatures from localized and metastatic PCa samples (lowest p-value <1.90E-14) from several independent studies. Further, the signatures “genes previously known to be methylated in prostate cancer” (p-value <1.40E-06) (Ongenaert et al. Nucleic Acids Res 36: D842-846 2008), and “gene ontology-tumor suppressor genes” (p-value<0.009) were significantly enriched (FIG. 18A). By contrast, PrEC cells did not share this enrichment and MCM analysis of PrEC-only methylated regions, revealed only concepts pertaining to histone modifications and were common to both PrEC and LNCaP MCM analysis (FIG. 18B). Finally, integration with RNA-Seq data revealed an association between gene repression and promoter methylation, globally by Gene Set Enrichment Analysis (GSEA) (FIG. 19) and upon specific evaluation of select genes (FIG. 20). For example, TIG1, GSTP1, CALML3, TASCTD2 and KCTD1 were methylated and repressed specifically in LNCaP, compared to SPON2 and GAGE genes that were methylated and repressed only in PrEC cells. HIC1 showed basal transcript expression and was methylated in both cell types.

Characterization of DNA Methylation in Prostate Cancer Tissues

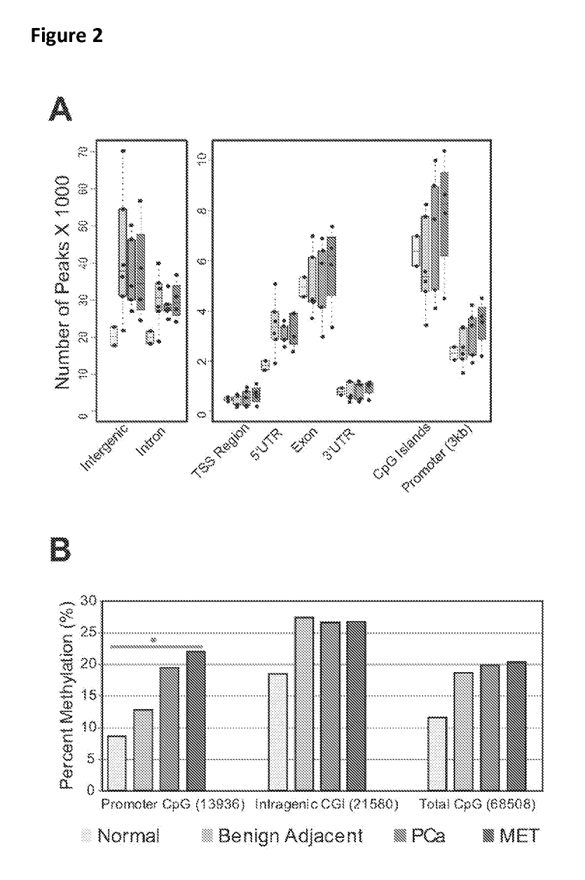

Having established the robustness of M-NGS to identify highly methylated regions in cell line models 17 prostate tissues (6 benign adjacent, 2 normal, 5 localized prostate cancer and 4 metastatic prostate cancer specimens) were next characterized (Table 3). A genome-wide assessment of both benign adjacent and cancer tissues showed a similar number of methylation events within intergenic and intronic regions (FIG. 2A). Of the total 68,508 CGIs present genome-wide, 18.5, 19.7 and 20.2 percent of all CGIs were methylated in benign, localized and metastatic cancer samples respectively (FIG. 2B). A significant increase in promoter-associated CGI methylation (Pearson's Chi Squared Test, p-value <2×10⁻¹⁶) paralleled prostate cancer progression (Benign 12.6%, Localized PCa 19.3% and Metastatic PCa 21.8%), whereas methylation of intragenic CGIs remained essentially unchanged (˜26.5%) among the three groups (FIG. 2B).

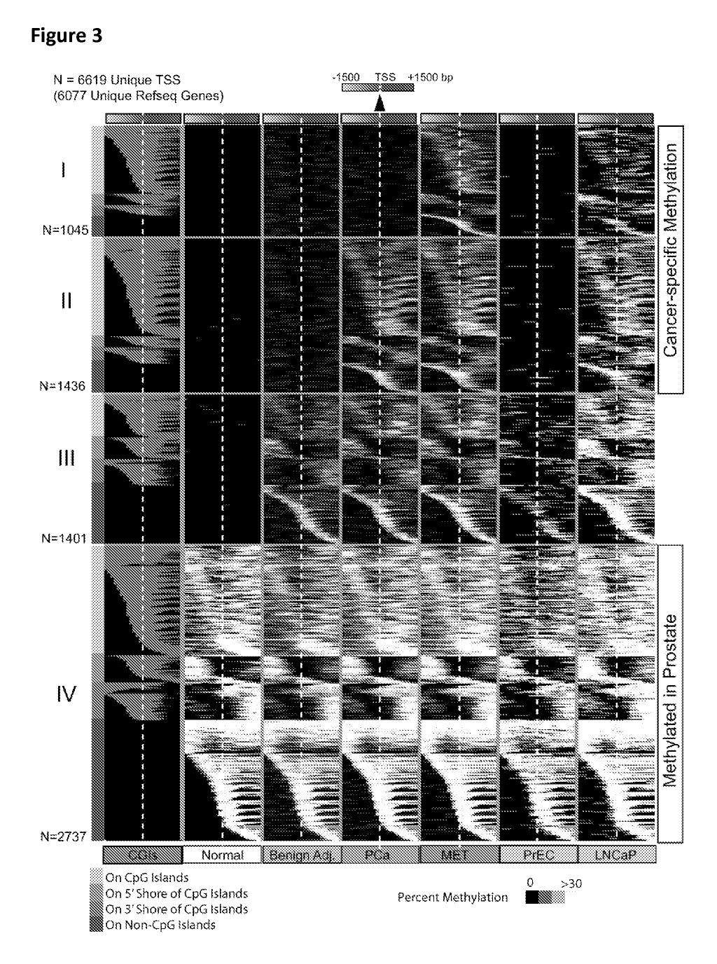

Next, 6619 promoter methylation events (within ±1500 bps flanking the transcriptional start site) present in either normal, benign adjacent, localized or metastatic prostate cancer samples were identified (FIG. 3 and Table 4). Of 6619 total methylation events, 2737 were found in all samples and 1401 of the remaining 3,882 were absent in normal prostate samples and PrEC cells but present in benign adjacent prostates. Nearly all of the 56 previously reported prostate cancer methylated regions from pubmeth.org and a recent study (Kron et al. PLoS One 4: e4830 2009) showed increased methylation in cancer tissues (Table 5).

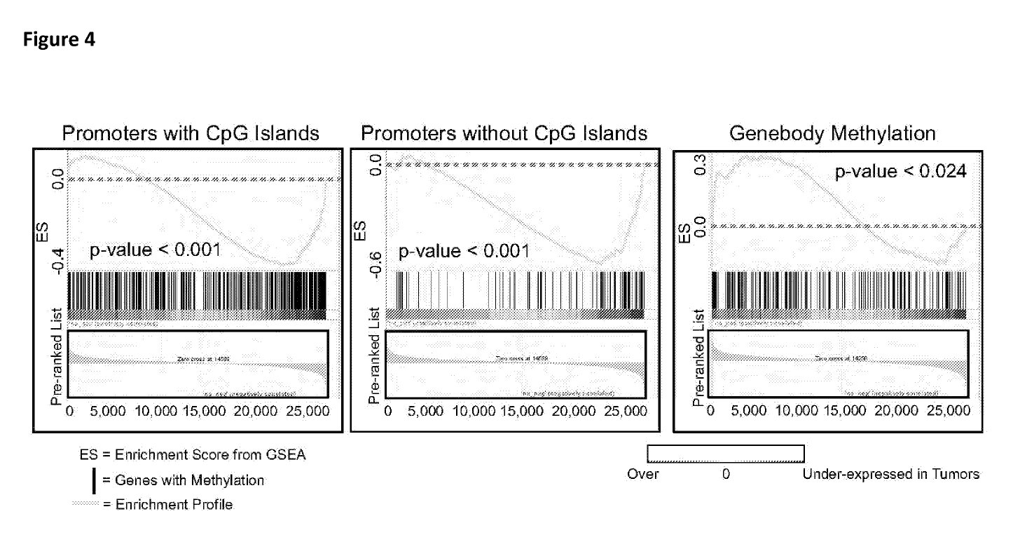

In order to identify DMRs with functional significance, promoter methylation events associated with transcriptional changes were examined. Promoters methylated in cancer were significantly associated with gene repression regardless of whether that promoter contained (p<0.001) or lacked (p<0.001) a CpG island by GSEA, while genes that displayed coding exon methylation tended to be overexpressed (p<0.024) (FIG. 4). Oncomine meta-analysis with 13 different prostate cancer gene expression dataset further supported methylated candidates association with gene repression. Several previously characterized methylation targets (GSTM2, GSTM1, S100A6, PYCARD and RARRES1) were present among this list, thereby validating the approach.

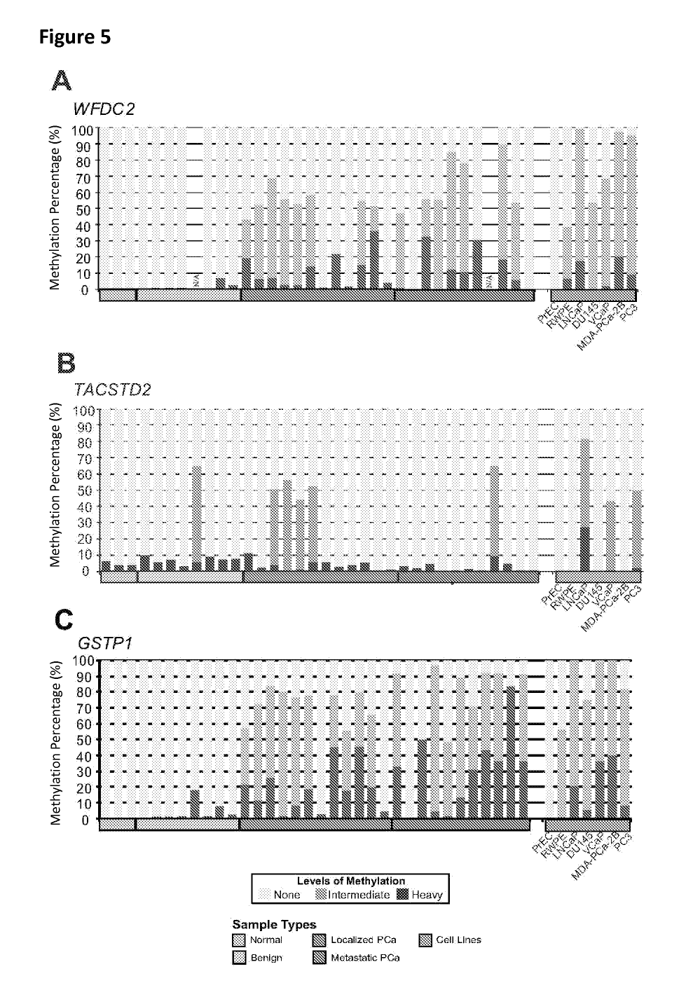

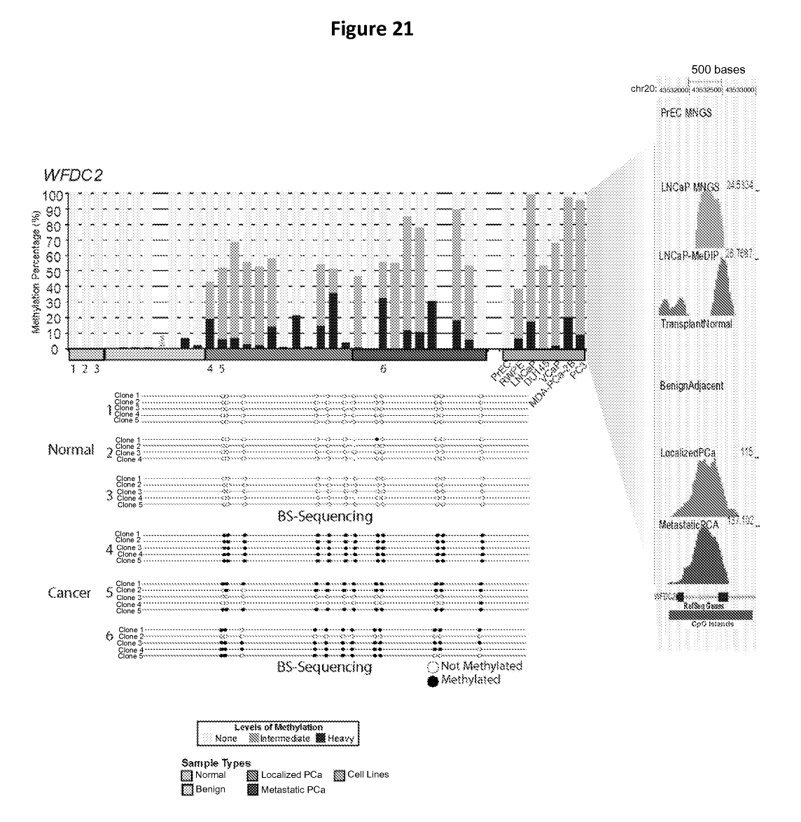

MethylProfiler PCR (Jaspers et al. Am J Respir Cell Mol Biol 43: 368-375 2010) was used as an independent evaluation of the methylation status of a novel target region in WFDC2 (WAP four-disulphide core domain protein 2, previously called HE4), a recently reported prostate methylation target TACSTD2 (Ibragimova et al. Cancer Prev Res (Phila) 3: 1084-1092 2010) and the well characterized GSTP1, all identified in this M-NGS study. WFDC2, which ranked 25^{th} in Oncomine meta-analysis, was methylated in 100% (6/6) of transformed prostate cell lines and 77% (17/22) of cancer tissues but not in benign tissues or PrEC (FIG. 5A). In addition, WFDC2 methylation in select samples was independently confirmed by bi-sulfite sequencing (FIG. 21). By comparison, the TACSTD2 promoter was less frequently methylated, with 21% (5/23) of cancer tissues and 9% (1/11) of benign tissues showing hypermethylation, and prostate cell lines similarly exhibited variable levels of methylation (FIG. 5B). In contrast, the well characterized GSTP1 promoter showed frequent methylation in cancer tissues (86%) and in all transformed cell lines (100%), similar to WFDC2 (FIG. 5C).

Regulation of Transcript Variant Expression by DNA Methylation

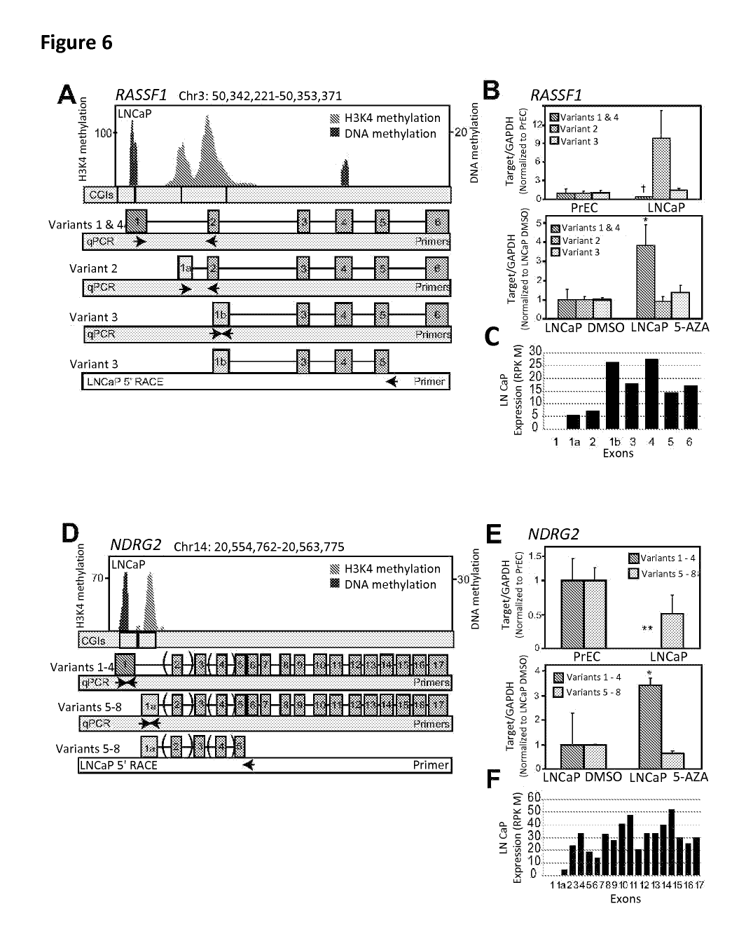

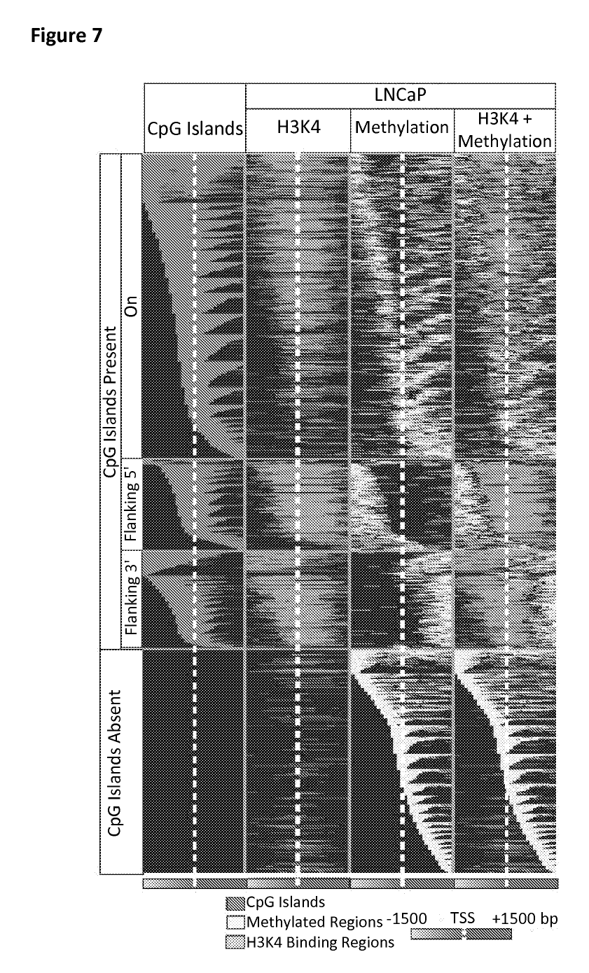

It was also observed that a subset of genes displayed selective promoter methylation in a transcript isoform-specific manner RASSF1, frequently inactivated by epigenetic alteration in human cancers (Dammann et al. Histol Histopathol 20: 645-663 2005), is comprised of three distinct variants. In LNCaP, DNA methylation-mediated silencing was observed of the longer transcript of RASSF1, variant-1, while the smaller isoform, variant-3, that codes for an N-terminal variant protein expressed in multiple cancer cell lines and tissues including PCa, retains high expression (FIG. 6 A, B) (Dammann et al. 2000, supra; Kuzmin et al. Cancer Res 62: 3498-3502 2002). Active transcription of variant-3 in LNCaP cells is supported by histone 3 lysine 4 trimethylation (H3K4me3) as observed in previously obtained ChIP-Seq data (Yu et al. 2010, supra), and 5′ Rapid Amplification of cDNA Ends (5′RACE) showed presence of shorter transcripts but not variant-1 in LNCaP (FIG. 6A). Isoform-specific methylation of variant-1 was confirmed by preferential re-expression of this transcript upon 5-Aza treatment of LNCaP cells (FIG. 6B). Segregation of epigenetic marks into distinct genomic regions was found in promoters containing CpG islands when we superimposed the promoter methylation and H3K4me3 ChIP-seq data from LNCaP cells (Yu et al. 2010, supra). (FIG. 7). While integration of other epigenetic marks is necessary for a full analysis, these data indicate that multiple epigenetic modifications may co-occur in distinct patterns to regulate transcript expression in cancer.

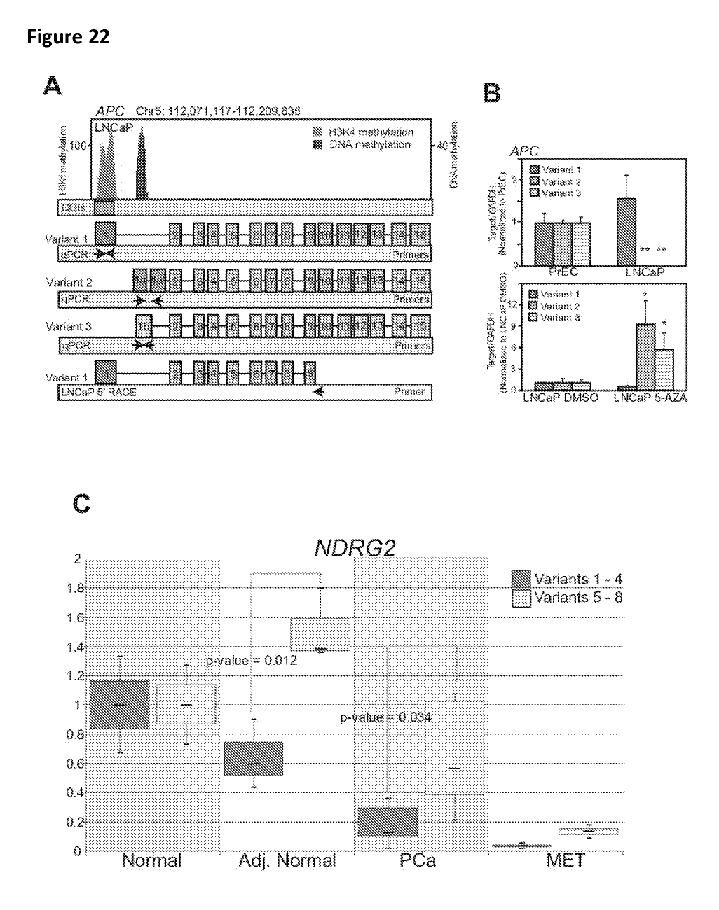

Since the M-NGS methodology accurately detected DNA methylation events of RASSF1, the data for differential methylation of transcript variants compared to H3K4me3 marks was queried, and 34 genes were identified in LNCaP that exhibit isoform-specific promoter methylation (Table 6). Two genes from this list (NDRG2 and APC) were validated (FIG. 6D and FIG. 22A). In both of these candidates, the transcript variants (variants 1-4 in NDRG2 and variant-2 and -3 in APC) showing DNA methylation were confirmed to be under-expressed in LNCaP cells compared to PrEC cells by qRT-PCR and 5′RACE (FIG. 6E and FIG. 19A). Furthermore, these variants were preferentially re-expressed upon 5-Aza treatment of LNCaP cells. To determine whether patient tissues demonstrated similar isoform-specific expression patterns, NDRG2 isoforms were tested in 2 normals, 3 adjacent normals, 5 localized PCa, and 2 metastatic samples by qRT-PCR. Similar to LNCaP cells, variants 1-4 were significantly under-expressed compared to variants 5-8 in localized PCa (p-value=0.034) and adjacent benign prostate (p-value=0.012), but not in normal (non-prostate cancer) tissues (FIG. 22B). In addition, previously obtained RNA-Seq data from LNCaP cells supported the above observation for RASSF1 and NDRG2 genes (FIGS. 6C and 6F).

Methylation Differences Between ETS-Positive and ETS-Negative Tissues

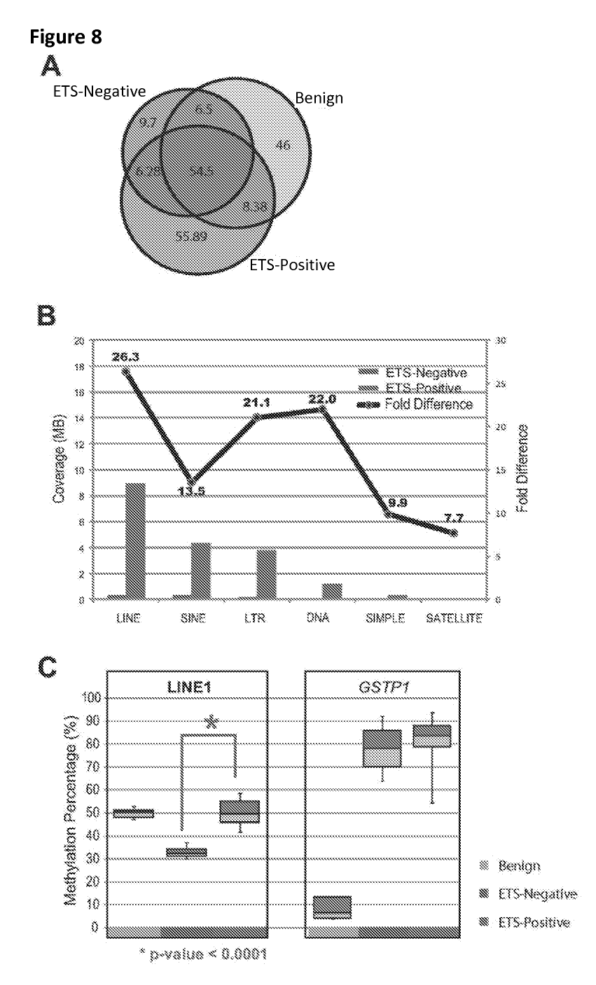

Transcription factor occupancy has a protective role in limiting the spread of DNA methylation into affected CpG islands (Gebhard et al. Cancer Res 70: 1398-1407 2010). In prostate cancer, gene fusions involving ETS transcription factors (most commonly ERG and ETV1) occur in approximately 40-50% of patients and serve as the most frequent genetic aberration in this disease (Kumar-Sinha et al. Nat Rev Cancer 8: 497-511 2008). DNA methylation differences between patients harboring or lacking an ETS gene fusion thus provides insights into transcriptional program of ERG in prostate cancer. The 5 ERG fusion-positive (ETS-Positive) patients and 4 fusion-negative (ETS-Negative) in the cohort were compared and more than 40 Mb of DMRs specifically associated with ETS positive samples were observed. The majority of DMRs in ETS-Negative samples were also shared with benign samples (FIG. 8A). ETS-Positive samples also contained higher repeat element methylation compared to ETS-Negative samples (FIG. 8B). In particular, assessment of global LINE-1 methylation by an independent pyrosequencing analysis on a prostate tissue cohort (n=20), revealed a significant decrease in LINE-1 element methylation (p-value <0.0001) in ETS-Negative compared to ETS-Positive samples (FIG. 8C). These data indicate that previous studies documenting global hypomethylation of LINE-1 elements in prostate cancer may miss subtleties present in different molecular subtypes of this disease.

Methylation Analysis of WFDC2 Gene Promoter

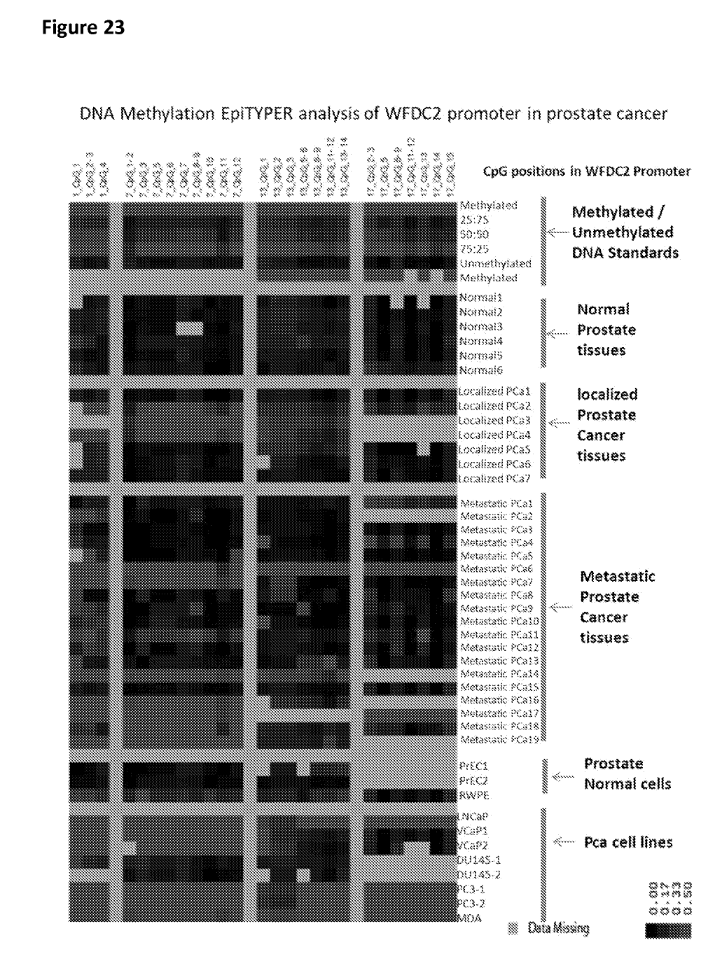

FIG. 23 shows DNA methylation analysis of WFDC2 gene promoter using EpiTYPER analysis. A gDNA sample cohort (n=42) that comprises prostate cell lines (10), Normal (6), localized PCa (7) and metastatic PCa (19) tissues. Bisulfite conversion was performed, specific genomic regions of interest in WFDC2 gene promoter were PCR amplified and the DNA methylation status of CG residues was characterized using EpiTYPER analysis. The results from this analysis is presented in FIG. 23, where the WFDC2 promoter region showed a high frequency of methylation compared to normal counterparts.

Four different PCR products (4 vertical column groups) that monitored 26 CG position in WFDC2 promoter were characterized by EpiTYPER analysis. Samples are represented in rows and CG position monitored is represented in columns. Unmethylated state and increasing degree of methylation at each position per sample is represented as a heatmap.

TABLE 1

| | | | | | |
|---|---|---|---|---|---|
| A4GALT | ABBA-1 | ABCC3 | ABCG4 | ABHD1 | ABHD9 |
| ACAA2 | ACADL | ACE | ACP1 | ACP5 | ACPL2 |
| ACSS1 | ACSS3 | ADAM32 | ADAMTS7 | ADAMTSL3 | ADM |
| ADORA2B | ADRA1A | ADRA2B | ADRA2C | ADRB1 | AGBL2 |
| ALDH1L1 | ALOX12 | ALOX15 | ALS2CR11 | AMOTL1 | AMOTL2 |
| ANKRD20A2 | ANKRD20A3 | ANKRD29 | ANKRD58 | ANUBL1 | ANXA2 |
| AOX1 | AP2A2 | AP3M2 | APCDD1 | AQP5 | ARHGAP4 |
| ARHGEF3 | ARL10 | ARL4C | ARL9 | ARNT2 | ARNTL |
| ARRB1 | ARSG | ARSI | ARTN | ARX | ASCL4 |
| ASTN2 | ATOH1 | ATP8A2 | ATP9B | AVPR1A | B4GALNT3 |
| BAG2 | BAHCC1 | BAMBI | BCL2L10 | BCL6B | BCL9L |
| BCR | BIRC3 | BLVRB | BNC2 | BOLA1 | BTBD14A |
| BTG4 | C10orf116 | C12orf56 | C13orf31 | C14orf169 | C14orf39 |
| C15orf26 | C17orf46 | C17orf64 | C17orf82 | C19orf23 | C19orf26 |
| C19orf55 | C1orf101 | C1orf104 | C1orf113 | C1orf183 | C1orf190 |
| C1orf194 | C1orf38 | C1orf59 | C1QL3 | C1QL4 | C20orf149 |
| C20orf85 | C21orf123 | C2orf39 | C2orf44 | C2orf55 | C3orf44 |
| C3orf52 | C3orf54 | C3orf55 | C3orf57 | C5orf35 | C6orf138 |
| C6orf145 | C6orf150 | C7orf41 | C8orf41 | C9orf125 | C9orf129 |
| C9orf25 | C9orf47 | C9orf64 | CABLES1 | CACNA1D | CACNA1H |
| CACNG4 | CALHM2 | CALML3 | CAMK1D | CARTPT | CBX4 |
| CBX8 | CCDC109B | CCDC11 | CCDC122 | CCDC134 | CCDC4 |
| CCDC67 | CCDC74B | CCDC87 | CCDC96 | CCK | CCND1 |
| CCNJL | CCS | CD14 | CD8A | CDC14B | CDH3 |
| CDO1 | CDYL | CEBPA | CGB7 | CHAD | CHKA |
| CHMP4A | CHST11 | CHST7 | CIDEB | CIRBP | CLDN10 |
| CLDN11 | CLEC14A | CLSTN1 | CMTM2 | CMTM3 | CNIH2 |
| CNNM2 | COASY | COCH | COL23A1 | COL7A1 | COL9A2 |
| CORO6 | CPM | CPNE7 | CPT1C | CR1L | CRABP2 |
| CRIP3 | CRYGD | CTBP2 | CTTNBP2 | CXCL16 | CXCR7 |
| CYB5R3 | CYP11A1 | CYP19A1 | CYP1A1 | CYP1B1 | CYP7B1 |
| DACT2 | DAP | DBNDD2 | DCDC2 | DCHS1 | DDAH2 |
| DDX60L | DEPDC7 | DFNB31 | DHRS3 | DHRS4L2 | DISP2 |
| DKFZp761E198 | DKK1 | DLEC1 | DLEU7 | DLX2 | DMC1 |
| DMN | DMRTA2 | DNAH10 | DNAH11 | DNAI1 | DNLZ |
| DOK6 | DPYSL5 | DSC2 | DSC3 | DSCR6 | DUSP4 |
| DUSP5 | DZIP1 | EBF2 | ECE2 | EDARADD | EDNRA |
| EFCAB4B | EFNA1 | EFNA3 | EFNA4 | EFS | EGR1 |
| EGR3 | EID3 | EIF4E3 | ELF4 | ELTD1 | EMID2 |
| ENC1 | ENO2 | EPAS1 | EPSTI1 | ERBB2IP | ESPNL |
| ETFB | ETNK2 | EXOC3L2 | F3 | FABP5 | FAM109B |
| FAM110C | FAM127C | FAM148B | FAM159A | FAM162B | FAM19A2 |
| FAM43A | FAM62C | FAM69B | FAM78A | FAM83G | FAM84A |
| FBN1 | FBP1 | FBXL11 | FBXO31 | FCHSD2 | FES |
| FGF18 | FGF22 | FHOD3 | FJX1 | FKBP11 | FKBP1B |
| FLJ21511 | FLJ36208 | FLJ40504 | FLJ43752 | FLJ45803 | FLOT1 |
| FN3K | FOSL1 | FOXB2 | FOXC1 | FOXD3 | FOXD4 |
| FOXD4L1 | FOXD4L2 | FOXD4L4 | FOXD4L6 | FOXG1 | FOXO1 |
| FOXP4 | FOXS1 | FRZB | FSCN2 | FSTL1 | FUCA2 |
| FZD1 | FZD7 | FZD8 | GALR1 | GAS2L1 | GATA6 |
| GBX2 | GCHFR | GCM2 | GDNF | GEMIN4 | GFI1 |
| GFRA3 | GGTLC1 | GIPC2 | GLDN | GLIS3 | GLP1R |
| GLS2 | GLT25D2 | GMPPA | GNA14 | GNE | GNG4 |
| GPR135 | GPR143 | GPR148 | GPR149 | GPR150 | GPR160 |
| GPR161 | GPR27 | GPR62 | GPR68 | GPRC5A | GPX3 |
| GSC | GSTM1 | GSTM2 | GSTM4 | GSTP1 | GSTT1 |
| HAPLN1 | HAS3 | HELT | HGC6.3 | HIF3A | HIST1H1A |
| HIST1H2AE | HIST1H2BG | HIST1H2BH | HIST1H2BI | HIST1H3A | HIST1H3E |
| HIST1H3F | HIST1H3G | HIST1H3I | HIST1H3J | HIST1H4A | HIST1H4F |
| HIST1H4I | HIST1H4J | HIST1H4L | HNF1A | HOXA10 | HOXA11 |
| HOXA6 | HOXA7 | HOXA9 | HOXB5 | HOXC10 | HOXC4 |
| HOXC9 | HPDL | HR | HS6ST1 | HSPA12A | HSPA2 |
| HSPA6 | HSPB6 | HTR7 | HTRA4 | HVCN1 | HYI |
| ICAM4 | IER3 | IFFO | IFIT5 | IGF2BP3 | IGFBP1 |
| IGFBP4 | IL17REL | INPP4B | IRF7 | IRX4 | IRX5 |
| ISL2 | ITGA4 | ITGB4 | ITPKA | ITPKB | JAKMIP1 |
| KAAG1 | KANK1 | KCNE3 | KCNG3 | KCNJ14 | KCNJ3 |
| KCNQ5 | KHDRBS2 | KHK | KIAA0644 | KIAA0888 | KIAA1024 |
| KIAA1324 | KIAA1383 | KIAA1529 | KIAA1754L | KIAA1975 | KIAA2013 |
| KIF1A | KIF3C | KIFC3 | KL | KLC1 | KLF11 |
| KLF4 | KLHDC1 | KLK4 | KLRG2 | LAMA3 | LAMB1 |
| LAPTM4B | LAYN | LBH | LBXCOR1 | LEPREL2 | LHB |
| LHX2 | LHX6 | LHX9 | LIMD2 | LMX1A | LOC116236 |
| LOC162073 | LOC201164 | LOC347475 | LOC388931 | LOC389118 | LOC389257 |
| LOC389813 | LOC54103 | LOC644096 | LOC653319 | LONRF3 | LOXL1 |
| LPAR3 | LRP1B | LRRC33 | LRRC4 | LRRK1 | LRRN1 |
| LSDP5 | LTB4R2 | LY6K | LYN | LYNX1 | MAP1B |
| MAP1LC3A | MAP6D1 | MAP7D2 | MAPK4 | MCCC2 | MDGA1 |
| ME3 | MEGF8 | MESP2 | MEX3B | MFAP3L | MGC21874 |
| MGC87631 | MICA | MKX | MLLT10 | MLXIPL | MOBKL2B |
| MOGAT1 | MPG | MPST | MRC2 | MRPS18A | MRPS6 |
| MT1A | MT1E | MTA2 | MTERFD3 | MX1 | MYF5 |
| N4BP2 | N4BP2L1 | N6AMT2 | NAV1 | NCAM2 | NCOA4 |
| NDN | NDRG2 | NDUFA4L2 | NDUFB4 | NEFL | NEFM |
| NFIL3 | NID1 | NIPSNAP3B | NKAPL | NKX2-1 | NKX2-5 |
| NKX3-2 | NKX6-1 | NLP | NMB | NOM1 | NOTCH3 |
| NPB | NPBWR1 | NPM2 | NPNT | NPR3 | NPY |
| NQO1 | NR2E1 | NRIP3 | NSUN7 | NT5DC2 | NT5DC3 |
| OCA2 | OLIG3 | ONECUT1 | ORAI3 | OSBPL9 | OVOL1 |
| P2RY2 | PACSIN3 | PAH | PATZ1 | PAX6 | PBX3 |
| PC-3 | PCDH10 | PCDH21 | PCDH7 | PCDHB4 | PCDHB5 |
| PCDHGA10 | PDE4C | PDE4DIP | PDK4 | PDLIM4 | PER1 |
| PFKP | PFN3 | PGAM2 | PGCP | PHF11 | PHF19 |
| PHPT1 | PIM3 | PIP5KL1 | PKN1 | PKN3 | PKP1 |
| PLA2G10 | PLCD1 | PLEC1 | PLLP | PNLDC1 | PON1 |
| POU3F1 | POU3F2 | POU3F3 | POU4F1 | PPARA | PPP1R14A |
| PPP1R3C | PPP1R3F | PPYR1 | PRLHR | PRPH | PRSS21 |
| PRSS3 | PRUNE2 | PSD3 | PSKH2 | PTCHD3 | PTGER2 |
| PTGER3 | PTGER4 | PTPRA | PTPRG | PTPRK | PTPRU |
| PTTG1IP | PXMP4 | PYDC1 | RAB11FIP3 | RAB6B | RAC3 |
| RAMP2 | RAPGEFL1 | RARRES1 | RARRES2 | RASGRF2 | RASGRP2 |
| RASL11B | RASSF1 | RASSF10 | RBM24 | RBM38 | RCN3 |
| RCSD1 | REC8 | REPIN1 | RGS16 | RGS2 | RGS3 |
| RHBDD1 | RHCG | RHEB | RIN3 | RLN2 | RPESP |
| RPL31 | RPL39L | RPP25 | RPRML | RPS6KA3 | RREB1 |
| RRN3 | RSHL1 | RSPH10B | RSPH10B2 | RTEL1 | RUNX3 |
| RUSC1 | RXRA | S100A6 | S1PR3 | SALL4 | SAMD13 |
| SARM1 | SCT | SCTR | SCUBE3 | SEMA3F | SEMA4C |
| SEMA5B | SEMA6A | SEPT9 | SERHL | SERPINB1 | SFN |
| SFRP4 | SFXN3 | SGIP1 | SH3PXD2A | SH3RF3 | SHC3 |
| SHC4 | SIM1 | SIPA1L3 | SIX1 | SIX2 | SIX6 |
| SLC12A2 | SLC16A11 | SLC22A16 | SLC22A17 | SLC24A3 | SLC25A20 |
| SLC27A2 | SLC27A3 | SLC29A1 | SLC35D1 | SLC35D3 | SLC38A1 |
| SLC4A11 | SLC5A3 | SLC7A5 | SLCO4C1 | SLIT1 | SMARCD3 |
| SNX22 | SOBP | SORBS3 | SOX11 | SOX13 | SOX15 |
| SOX9 | SP5 | SPAG16 | SPAG6 | SPDYA | SPG3A |
| SPHK1 | SPIN2A | SPIRE1 | SPRN | SPTBN4 | SSBP4 |
| SSPN | ST6GALNAC3 | STX16 | STXBP6 | SYCN | SYK |
| SYNJ2 | SYT9 | TACC2 | TACR3 | TACSTD2 | TAF4 |
| TBC1D1 | TBKBP1 | TBPL2 | TBR1 | TBX1 | TCEA2 |
| TCF12 | TDRD1 | TDRD12 | TEAD3 | TF | TGFB2 |
| THBD | THRB | TJP2 | TLE3 | TLL2 | TMEM106A |
| TMEM22 | TMEM220 | TMEM26 | TMEM30B | TMEM42 | TMEM45B |
| TMPRSS12 | TNFRSF4 | TNFRSF6B | TNRC18 | TOX2 | TP53I13 |
| TP73 | TPPP3 | TRAF5 | TRHDE | TRIB1 | TRIM36 |
| TRIM59 | TSSK2 | TSSK3 | TST | TTC29 | TWIST1 |
| TXNRD1 | UAP1L1 | UBE2J2 | UCHL1 | UCN | UCP2 |
| UHRF2 | ULBP1 | UNC93B1 | UNQ9438 | UPB1 | USP44 |
| UST | VAMP5 | VASH2 | VAX2 | VEGFB | VGLL4 |
| VIM | VIPR1 | VTN | VWCE | WDR40B | WDR52 |
| WDR8 | WDR82 | WFDC2 | WNK3 | WNT2 | WNT2B |
| WWC3 | WWTR1 | XK | YBX1 | YDJC | ZAR1 |
| ZC3H12C | ZDHHC1 | ZIC2 | ZIC5 | ZIK1 | ZMYM2 |
| ZMYM5 | ZMYND15 | ZNF236 | ZNF331 | ZNF518B | ZNF594 |
| ZNF595 | ZNF703 | ZNF706 | ZNF710 | ZNF718 | ZP3 |
| ZPBP2 | ZSWIM2 | | | | |

TABLE 2

| | | | | | | | Amplicon |
|---|---|---|---|---|---|---|---|
| | Genomic | Accession | | | | | Size |
| Gene | Location | Number | Type | Forward Primer | Reverse Primer | Applications | (bp) |
| RASSF1A | chr3:50353011- | N/A | LNCaP- | GTAGTTTAATG | CTACACCCAAA | BS-sequencing | 188 |
| | 50353198 | | Hyper | AGTTTAGGTTT | TTTCCATTAC | | |
| | | | | TTT | (SEQ ID NO: 32) | | |
| | | | | (SEQ ID NO: 1) | | | |
| KCTD1 | chr18:22381178- | N/A | LNCaP- | GTTTTGGAGA | AAACTCTTTTA | BS-sequencing | 373 |
| | 22381550 | | Hyper | GTATTTGTATT | AACTAACCATA | | |
| | | | | (SEQ ID NO: 2) | AAC | | |
| | | | | | (SEQ ID NO: 33) | | |
| CHMP4A | chr14:28313174- | N/A | LNCaP- | GAGTTTTTTTG | ACAACTAAAAT | BS-sequencing | 238 |
| | 28313411 | | Hyper | TTTTAGAGTGG | TACTTCTCCAC | | |
| | | | | TTGA | ACC | | |
| | | | | (SEQ ID NO: 3) | (SEQ ID NO: 34) | | |
| APC | chr5:112101275- | N/A | LNCaP- | GGGTTAGGGT | ACACCTCCATT | BS-sequencing | 229 |
| | 112101503 | | Hyper | TAGGTAGGTT | CTATCTCCAAT | | |
| | | | | GT | AAC | | |
| | | | | (SEQ ID NO: 4) | (SEQ ID NO: 35) | | |
| CDKN2A | chr9:21960825- | N/A | LNCaP- | ATTTTTTGTGT | AAACCTAACTA | BS-sequencing | 191 |
| | 21961015 | | Hyper | TGGAAAATGAA | AAAAACTAAAC | | |
| | | | | TGT | CATC | | |
| | | | | (SEQ ID NO: 5) | (SEQ ID NO: 36) | | |
| SHC1 | chr1:153209107- | N/A | LNCaP- | GAATGGAGAG | AAAAACAACCA | BS-sequencing | 238 |
| | 153209344 | | Hyper | GAGGATGTTTA | TACCAAACTCA | | |
| | | | | TTTAT | AAC | | |
| | | | | (SEQ ID NO: 6) | (SEQ ID NO: 37) | | |
| LAMC2 | chr1:181421688- | N/A | LNCaP- | TGTTTATTTTT | ACCTATAAATA | BS-sequencing | 244 |
| | 181421931 | | Hyper | GTGAATTTGTT | AAAAAAACCCA | | |
| | | | | TTG | ACC | | |
| | | | | (SEQ ID NO: 7) | (SEQ ID NO: 38) | | |
| TINAGL1 | chr1:31814575- | N/A | LNCaP- | ATTTTGTAAGT | TTCTATTCCTA | BS-sequencing | 344 |
| | 31814918 | | Hyper | TTATGAGTTGT | TATCCCTCTAT | | |
| | | | | GGG | CCC | | |
| | | | | (SEQ ID NO: 8) | (SEQ ID NO: 39) | | |
| TSPAN1 | chr1:46418579- | N/A | LNCaP- | TGTTTTATTTTT | CACACACCACT | BS-sequencing | 224 |
| | 46418802 | | Hyper | GGGTTGTTTTT | ACTCACCTACA | | |
| | | | | TT | AAC | | |
| | | | | (SEQ ID NO: 9) | (SEQ ID NO: 40) | | |
| CALML3 | chr10:5556581- | N/A | LNCaP- | TTATTTAAGGG | ATTTAAACAAA | BS-sequencing | 250 |
| | 5556830 | | Hyper | AAGAAAGGGT | AATCCAAAACC | | |
| | | | | ATTG | TAC | | |
| | | | | (SEQ ID NO: 10) | (SEQ ID NO: 41) | | |
| SPON2-1 | chr4:1156947- | N/A | PrEC- | TAGTTTATATG | AAAACCCCTAA | BS-sequencing | 320 |
| | 1157266 | | Hyper | TTGGAAGTGG | AAAAACTCTAC | | |
| | | | | TTGG | ACC | | |
| | | | | (SEQ ID NO: 11) | (SEQ ID NO: 42) | | |
| SPON2-2 | chr4:1157164- | N/A | PrEC- | TAGGAAGAGTT | CATTAACAAAA | BS-sequencing | 325 |
| | 1157488 | | Hyper | ATAGAAAGGG | TTCCAAACATC | | |
| | | | | GGTT | AAA | | |
| | | | | (SEQ ID NO: 12) | (SEQ ID NO: 43) | | |
| PPP1R3C | chr10:93382583- | N/A | LNCaP | TTTTTTGGGGT | AACCCTCAATC | BS-sequencing | 233 |
| | 93382815 | | Hyper | GTTTATTTTTA | TCTCCCAAC | | |
| | | | | GAG | (SEQ ID NO: 44) | | |
| | | | | (SEQ ID NO: 13) | | | |
| AMT | chr3:49434492- | N/A | LNCaP | AATAGGGAGG | AAACCCTAATA | BS-sequencing | 251 |
| | 49434742 | | Hyper | AAGGTTTGATT | ACACTAAACCC | | |
| | | | | AGAT | AAC | | |
| | | | | (SEQ ID NO: 14) | (SEQ ID NO: 45) | | |
| NTN4 | chr12:94707434- | N/A | LNCaP | GTTTAAGAAAG | AAAAAACCAAA | BS-sequencing | 271 |
| | 94707704 | | Hyper | TTAGGATGGG | TAAAAACACCT | | |
| | | | | AGTG | AAAAC | | |
| | | | | (SEQ ID NO: 15) | (SEQ ID NO: 46) | | |
| AOX1 | chr2:201158884- | N/A | LNCaP | GAGTTTTTGGT | CAAATCTAAAA | BS-sequencing | 274 |
| | 201159157 | | Hyper | AAAGAGTTTAG | ACAAAAAAAAC | | |
| | | | | GAA | CC | | |
| | | | | (SEQ ID NO: 16) | (SEQ ID NO: 47) | | |
| C9orf125 | chr9:103289105- | N/A | LNCaP | ATTGTTTAGGT | CTTTTCCCTAA | BS-sequencing | 244 |
| | 103289348 | | Hyper | TGGTTAGTTTA | CTACCCATCAA | | |
| | | | | TTT | TTA | | |
| | | | | (SEQ ID NO: 17) | (SEQ ID NO: 48) | | |
| C9orf125 | chr9:103288104- | N/A | LNCaP | GTATGGTGAG | ACTAAATCCAA | BS-sequencing | 242 |
| | 103288345 | | Hyper | ATTATTATTGG | AACCCTAAACA | | |
| | | | | AGT | TC | | |
| | | | | (SEQ ID NO: 18) | (SEQ ID NO: 49) | | |
| NAP1L5 | chr4:89837763- | N/A | LNCaP | GGGTTTTTTAG | CAAAATCTCTC | BS-sequencing | 241 |
| | 89838003 | | and PrEC | TTATTTGATTA | TAAACCAACTC | | |
| | | | Hyper | GT | (SEQ ID NO: 50) | | |
| | | | | (SEQ ID NO: 19) | | | |
| NAP1L5 | chr4:89837763- | N/A | LNCaP | GGGTTTTTTAG | AAAACCCTCCTAA | BS-sequencing | 203 |
| | 89837965 | | and PrEC | TTATTTGATTA | ACCTCTAC | | |
| | | | Hyper | GT | (SEQ ID NO: 51) | | |
| | | | | (SEQ ID NO: 20) | | | |
| MYC | chr8:128815497- | N/A | Negative | TTTGTTTTTGT | ATTACTCCTAC | BS-sequencing | 214 |
| | 128815710 | | control | TTTTATTTGATT | CTCCAAACCTT | | |
| | | | | TT | TAC | | |
| | | | | (SEQ ID NO: 21) | (SEQ ID NO: 52) | | |
| APC | N/A | NM_001127511 | Variant 1 | TCAGTTCTCGG | TCCTTGGCTAC | qPCR | |
| | | | | GTCCTGGAG | CCTTGGAC | | |
| | | | | (SEQ ID NO: 22) | (SEQ ID NO: 53) | | |
| APC | N/A | NM_001127510 | Variant 2 | GTGTCACTGG | TAGATT | qPCR | |
| | | | | AGACAGAATG | CACATCAGCC | | |
| | | | | GA | ATCTGC | | |
| | | | | (SEQ ID NO: 23) | (SEQ ID NO: 54) | | |
| APC | N/A | NM_000038 | Variant 3 | AGGGTGTCAC | CTACCCTTGGA | qPCR | |
| | | | | TGGAGACAGA | CCCCATTTC | | |
| | | | | AT | (SEQ ID NO: 55) | | |
| | | | | (SEQ ID NO: 24) | | | |
| GAPDH | N/A | NM_002046 | N/A | CTGAACGGGA | TTACTCCTTGG | qPCR | |
| | | | | AGCTCACTGG | AGGCCATGTG | | |
| | | | | CA | GG | | |
| | | | | (SEQ ID NO: 25) | (SEQ ID NO: 56) | | |
| GSTP1 | N/A | NM_000852 | N/A | GACTTGCTGCT | AGGTTCACGTA | qPCR | |
| | | | | GATCCATGA | CTCAGGGGAG | | |
| | | | | (SEQ ID NO: 26) | (SEQ ID NO: 57) | | |
| NDRG2 | N/A | NM_210535 | Variant 1 | CCTTGTTGTCC | ACAGTGGCTT | qPCR | |
| | | NM_016250 | Variant 2 | AACTTCTCCC | CTCCTCTGTGA | | |
| | | NM_201536 | Variant 3 | (SEQ ID NO: 27) | T | | |
| | | NM_210537 | Variant 4 | | (SEQ ID NO: 58) | | |
| NDRG2 | N/A | NM_201538 | Variant 5 | GAGTCAAAGG | ACAGTGGCTT | qPCR | |
| | | NM_201539 | Variant 6 | CAAGTGAAGG | CTCCTCTGTGA | | |
| | | NM_201540 | Variant 7 | TG | T | | |
| | | NM_201541 | Variant 8 | (SEQ ID NO: 28) | (SEQ ID NO: 59) | | |
| RASSF1 | N/A | NM_007182 | Variant 1 | GACCTCTGTG | GGCAGGTGAA | qPCR | |
| | | NM_170714 | Variant 4 | GCGACTTCAT | CTTGCAATC | | |
| | | | | (SEQ ID NO: 29) | (SEQ ID NO: 60) | | |
| RASSF1 | N/A | NM_170712 | Variant 2 | AGGTGGCCAA | AACAGTCCAG | qPCR | |
| | | | | CATTAGAGTCC | GCAGACGAG | | |
| | | | | (SEQ ID NO: 30) | (SEQ ID NO: 61) | | |
| RASSF1 | N/A | NM_170713 | Variant 3 | CTTCTTTCGAA | TCCGAGTCCG | qPCR | |
| | | | | ATGACCTGGA | AGTCCTCTT | | |
| | | | | G | (SEQ ID NO: 62) | | |
| | | | | (SEQ ID NO: 31) | | | |
| APC | N/A | All Variants | 1st round | N/A | TGCAATGGCC | 5′RACE | |
| | | | | | TGTAGTCCCC | | |
| | | | | | CTAGT | | |
| | | | | | (SEQ ID NO: 63) | | |
| | | | nested | N/A | AGCCTTCGAG | 5′RACE | |
| | | | PCR | | GTGCAGAGTG | | |
| | | | | | TGTGCT | | |
| | | | | | (SEQ ID NO: 64) | | |
| NDRG2 | N/A | All variants | 1st round | N/A | CCAGGGGCAT | 5′RACE | |
| | | | | | CCACATGAAC | | |
| | | | | | CCGCA | | |
| | | | | | (SEQ ID NO: 65) | | |
| | | | nested | N/A | CGCTGGGCGT | 5′RACE | |
| | | | PCR | | TTGGGTTTGG | | |
| | | | | | GGGT | | |
| | | | | | (SEQ ID NO: 66) | | |
| RASSF1 | N/A | All Variants | 1st round | N/A | CAGAGCCATA | 5′RACE | |
| | | | | | CCTGGCTACA | | |
| | | | | | C | | |
| | | | | | (SEQ ID NO: 67) | | |
| | | | nested | N/A | GCCGCAGGGG | 5′RACE | |
| | | | PCR | | CTGCTCATCAT | | |
| | | | | | CCA | | |
| | | | | | (SEQ ID NO: 68) | | |

TABLE 3

| FC ID | | 6159FAAXX.s8 | 429C3AAXX.s7 | 429C3AAXX.s4 | 6159FAAXX.s2 | 61M19AAXX.s3 | 61LWKAAXX.s5 | 61M19AAXX.s4 | 61LWKAAXX.s7 | 61LWKAAXX.s6 | 61LWKAAXX.s8 |
|---|---|---|---|---|---|---|---|---|---|---|---|
| Tissue Group | | Transplant | | Benign Adjacent | | | | | | | |
| | | Normal | | | | | | | | | |
| Tissue ID | | N31 | N32 | N19 | N21 | N-t29 | | N-t40 | | N-t32 | N-t21 |
| Gel Cut Position (bp) | | 350-450 | | | | | | | | | |
| Methylplex Product Used | | 5 μg | | | | | | | | | |
| (ugs) | | | | | | | | | | | |
| Method | | M-NGS | | | | | | | | | |
| HMM Analysis (# of | | 52563 | 42782 | 82320 | 69350 | 85008 | 108551 | 75379 | 73985 | 120596 | 46744 |
| Peaks) | | | | | | | | | | | |
| Coverage (Mb) | | 38.87 | 28.22 | 56.4 | 53.24 | 36.93 | 47.59 | 34.71 | 33.59 | 52.16 | 21.35 |
| Overlap with CpG | | 7829 | 6845 | 7557 | 8475 | 4698 | 5653 | 4946 | 4613 | 5558 | 3432 |
| Islands | | | | | | | | | | | |
| CGI Coverage Overlap | | 3.24 | 2.76 | 2.67 | 3.39 | 1.89 | 2.33 | 2.08 | 1.92 | 2.26 | 1.39 |
| (Mb) | | | | | | | | | | | |
| Overlap with Gene | | 2560 | 2050 | 3342 | 3312 | 2078 | 2503 | 2149 | 2012 | 2560 | 1526 |
| Promoter (out of 20099 | | (1.87) | (1.35) | (2.37) | (2.49) | (0.899) | (1.14) | (1.01) | (0.917) | (1.13) | (0.661) |
| genes, 17.61 Mb) | | | | | | | | | | | |
| Total | CG count | 34440051 | 17564762 | 16454794 | 24785796 | 28112895 | 30055602 | 32093448 | 34095287 | 25393375 | 31481821 |
| | # sequence | 24024896 | 15186618 | 15113798 | 21129187 | 24342833 | 26490815 | 24614718 | 27187078 | 25452611 | 25535899 |
| | CG rate (per kb) | 49.43 | 39.88 | 37.54 | 40.45 | 39.82 | 39.12 | 44.96 | 43.24 | 34.40 | 42.51 |

| | | | | | | | | | | |
|---|---|---|---|---|---|---|---|---|---|---|
| read length | 40 | 40 | 40 | 40 | 40 | 40 | 40 | 40 | 40 | 40 |
| ETS-Status | NA | NA | NA | NA | NA | NA | NA | NA | NA | NA |

| FC ID | 6159FAAXX.s4 | ,61M19AAXX.s7 | 61M19AAXX.s8 | 6159FAAXX.s3 | 429C3AAXX.s5 |
|---|---|---|---|---|---|
| Tissue Group | Localized PCA | | | | |
| Tissue ID | T45 | T54 | T59 | T44 | T43 |
| Gel Cut Position (bp) | 350-450 | | | | |
| Methylplex Product Used | 5 μg | | | | |
| (ugs) | | | | | |
| Method | M-NGS | | | | |
| HMM Analysis (# of | 61462 | 84972 | 83474 | 67916 | 78533 |
| Peaks) | | | | | |
| Coverage (Mb) | 45.88 | 35.85 | 36.67 | 51.65 | 49.86 |
| Overlap with CpG Islands | 9978 | 4106 | 4843 | 10937 | 7628 |
| CGI Coverage Overlap | 4.06 | 1.56 | 1.96 | 4.52 | 2.49 |
| (Mb) | | | | | |
| Overlap with Gene | 3726 (2.70) | 1932 (0.762) | 2243 (0.977) | 4222 (3.18) | 3405 (2.22) |
| Promoter (out of 20099 | | | | | |
| genes, 17.61 Mb) | | | | | |

| | | | | | | |
|---|---|---|---|---|---|---|
| Total | CG count | 29418521 | 22990149 | 29849171 | 28316245 | 15954780 |
| | # sequence | 22483683 | 23388750 | 24893052 | 22290447 | 14534922 |
| | CG rate (per kb) | 45.11 | 33.89 | 41.34 | 43.80 | 37.85 |

| read length | 40 | 40 | 40 | 40 | 40 |
|---|---|---|---|---|---|
| ETS-Status | ETS- | ETS-Positive | ETS-Positive | ETS- | ETS- |
| | Negative | | | Positive | Negative |
| FC ID | 6159FAAXX.s6 | 429C3AAXX.s6 | 61M19AAXX.s6 | 6159FAAXX.s5 | 429C3AAXX.s8 |

| | | |
|---|---|---|
| Tissue Group | Metastasized PCA | LNCaP |

| Tissue ID | M18 | M15 | M17 | M16 | cancer cell |
|---|---|---|---|---|---|
| | | | | | line |
| Gel Cut Position (bp) | 350-450 | | | | |
| Methylplex Product Used | 5 μg | | | | |
| (ugs) | | | | | |
| Method | M-NGS | | | | |
| HMM Analysis (# of | 68759 | 87247 | 95656 | 58196 | 81812 |
| Peaks) | | | | | |
| Coverage (Mb) | 51.65 | 52.97 | 40.16 | 44.06 | 51.91 |
| Overlap with CpG Islands | 11566 | 7371 | 4498 | 9564 | 8013 |
| CGI Coverage Overlap | 4.9 | 2.34 | 1.77 | 4.11 | 2.64 |
| (Mb) | | | | | |
| Overlap with Gene | 4489 (3.52) | 3772 (2.39) | 2199 (0.934) | 3539 (2.58) | 4265 (2.83) |
| Promoter (out of 20099 | | | | | |
| genes, 17.61 Mb) | | | | | |

| | | | | | | |
|---|---|---|---|---|---|---|
| Total | CG count | 28742701 | 14754630 | 21980747 | 33149324 | 13298874 |
| | # sequence | 22577109 | 14549842 | 23494735 | 23883795 | 14909301 |
| | CG rate (per kb) | 43.90 | 34.97 | 32.26 | 47.86 | 30.76 |

| | | | | | |
|---|---|---|---|---|---|
| read length | 40 | 40 | 40 | 40 | 40 |
| ETS-Status | ETS-Negative | ETS-Positive | ETS-Positive | ETS-Negative | ETS-Positive |

TABLE 4

| | HG18 Chormosomal | Gene Name and Chromosomal location |
|---|---|---|
| Gene Name | Location | (HG18) |
| MMP23B | Chr1: 1555922-1558922 | MMP23B, Chr1: 1555922-1558922 |
| RGS20 | Chr8: 54954617-54957617 | RGS20, Chr8: 54954617-54957617 |
| POU3F2 | Chr6: 99387800-99390800 | POU3F2, Chr6: 99387800-99390800 |
| POLE | Chr12: 131772518-131775518 | POLE, Chr12: 131772518-131775518 |
| SORCS3 | Chr10: 106389348-106392348 | SORCS3, Chr10: 106389348-106392348 |
| TRIM71 | Chr3: 32833013-32836013 | TRIM71, Chr3: 32833013-32836013 |
| KIF21B | Chr1: 199257951-199260951 | KIF21B, Chr1: 199257951-199260951 |
| NKX6-2 | Chr10: 134448027-134451027 | NKX6-2, Chr10: 134448027-134451027 |
| PBX3 | Chr9: 127547986-127550986 | PBX3, Chr9: 127547986-127550986 |
| FOXC2 | Chr16: 85156942-85159942 | FOXC2, Chr16: 85156942-85159942 |
| UBE2I | Chr16: 1297680-1300680 | UBE2I, Chr16: 1297680-1300680 |
| NTRK3 | Chr15: 86599165-86602165 | NTRK3, Chr15: 86599165-86602165 |
| WNT2 | Chr7: 116749079-116752079 | WNT2, Chr7: 116749079-116752079 |
| FAT4 | Chr4: 126455516-126458516 | FAT4, Chr4: 126455516-126458516 |
| LOC339344 | Chr19: 51096202-51099202 | LOC339344, Chr19: 51096202-51099202 |
| NOL4 | Chr18: 30055944-30058944 | NOL4, Chr18: 30055944-30058944 |
| BARHL2 | Chr1: 90953882-90956882 | BARHL2, Chr1: 90953882-90956882 |
| NETO1 | Chr18: 68684290-68687290 | NETO1, Chr18: 68684290-68687290 |
| SV2C | Chr5: 75413560-75416560 | SV2C, Chr5: 75413560-75416560 |
| USF2 | Chr19: 40450235-40453235 | USF2, Chr19: 40450235-40453235 |
| ZBTB1 | Chr14: 64039544-64042544 | ZBTB1, Chr14: 64039544-64042544 |
| ASCL1 | Chr12: 101874081-101877081 | ASCL1, Chr12: 101874081-101877081 |
| ATP5D | Chr19: 1191248-1194248 | ATP5D, Chr19: 1191248-1194248 |
| CBX4 | Chr17: 75426308-75429308 | CBX4, Chr17: 75426308-75429308 |
| MGC21874 | Chr4: 7094556-7097556 | MGC21874, Chr4: 7094556-7097556 |
| KCNK12 | Chr2: 47649474-47652474 | KCNK12, Chr2: 47649474-47652474 |
| RSL1D1 | Chr16: 11851443-11854443 | RSL1D1, Chr16: 11851443-11854443 |
| LBX1 | Chr10: 102977207-102980207 | LBX1, Chr10: 102977207-102980207 |
| XKR4 | Chr8: 56176070-56179070 | XKR4, Chr8: 56176070-56179070 |
| ZBTB25 | Chr14: 64038807-64041807 | ZBTB25, Chr14: 64038807-64041807 |
| HBM | Chr16: 154472-157472 | HBM, Chr16: 154472-157472 |
| CDCA3 | Chr12: 6829217-6832217 | CDCA3, Chr12: 6829217-6832217 |
| GAD1 | Chr2: 171379945-171382945 | GAD1, Chr2: 171379945-171382945 |
| USP5 | Chr12: 6830045-6833045 | USP5, Chr12: 6830045-6833045 |
| USP5 | Chr12: 6830051-6833051 | USP5, Chr12: 6830051-6833051 |
| GPR50 | Chr23: 150094216-150097216 | GPR50, Chr23: 150094216-150097216 |
| PASK | Chr2: 241736051-241739051 | PASK, Chr2: 241736051-241739051 |
| HEY2 | Chr6: 126110924-126113924 | HEY2, Chr6: 126110924-126113924 |
| C20orf82 | Chr20: 13148917-13151917 | C20orf82, Chr20: 13148917-13151917 |
| NDUFB10 | Chr16: 1948017-1951017 | NDUFB10, Chr16: 1948017-1951017 |
| LOC401089 | Chr3: 140147265-140150265 | LOC401089, Chr3: 140147265-140150265 |
| HMX2 | Chr10: 124896127-124899127 | HMX2, Chr10: 124896127-124899127 |
| PPP1R7 | Chr2: 241737074-241740074 | PPP1R7, Chr2: 241737074-241740074 |
| CDH11 | Chr16: 63711920-63714920 | CDH11, Chr16: 63711920-63714920 |
| TBX18 | Chr6: 85529118-85532118 | TBX18, Chr6: 85529118-85532118 |
| NUP85 | Chr17: 70711691-70714691 | NUP85, Chr17: 70711691-70714691 |
| FOS | Chr14: 74813783-74816783 | FOS, Chr14: 74813783-74816783 |
| CACNA1G | Chr17: 45991947-45994947 | CACNA1G, Chr17: 45991947-45994947 |
| TBX5 | Chr12: 113329130-113332130 | TBX5, Chr12: 113329130-113332130 |
| PNPLA7 | Chr9: 139563307-139566307 | PNPLA7, Chr9: 139563307-139566307 |
| CCDC148 | Chr2: 159019960-159022960 | CCDC148, Chr2: 159019960-159022960 |
| SLITRK5 | Chr13: 87121370-87124370 | SLITRK5, Chr13: 87121370-87124370 |
| HOXA3 | Chr7: 27118641-27121641 | HOXA3, Chr7: 27118641-27121641 |
| PDE5A | Chr4: 120766390-120769390 | PDE5A, Chr4: 120766390-120769390 |
| BLVRB | Chr19: 45662065-45665065 | BLVRB, Chr19: 45662065-45665065 |
| MYD88 | Chr3: 38153508-38156508 | MYD88, Chr3: 38153508-38156508 |
| HOXC5 | Chr12: 52711598-52714598 | HOXC5, Chr12: 52711598-52714598 |
| HOXA11 | Chr7: 27189860-27192860 | HOXA11, Chr7: 27189860-27192860 |
| ARHGEF1 | Chr19: 47078785-47081785 | ARHGEF1, Chr19: 47078785-47081785 |
| SPTBN4 | Chr19: 45663465-45666465 | SPTBN4, Chr19: 45663465-45666465 |
| GRWD1 | Chr19: 53639373-53642373 | GRWD1, Chr19: 53639373-53642373 |
| RNF41 | Chr12: 54900502-54903502 | RNF41, Chr12: 54900502-54903502 |
| MOGAT3 | Chr7: 100629522-100632522 | MOGAT3, Chr7: 100629522-100632522 |
| RNF41 | Chr12: 54900471-54903471 | RNF41, Chr12: 54900471-54903471 |
| RBM6 | Chr3: 49951095-49954095 | RBM6, Chr3: 49951095-49954095 |
| SIM1 | Chr6: 101016772-101019772 | SIM1, Chr6: 101016772-101019772 |
| ACAA1 | Chr3: 38152119-38155119 | ACAA1, Chr3: 38152119-38155119 |
| CNTN4 | Chr3: 2115746-2118746 | CNTN4, Chr3: 2115746-2118746 |
| ACTR6 | Chr12: 99117204-99120204 | ACTR6, Chr12: 99117204-99120204 |
| FOXD2 | Chr1: 47672775-47675775 | FOXD2, Chr1: 47672775-47675775 |
| GMPPA | Chr2: 220070356-220073356 | GMPPA, Chr2: 220070356-220073356 |
| CPS1 | Chr2: 211049153-211052153 | CPS1, Chr2: 211049153-211052153 |
| HAPLN1 | Chr5: 83051152-83054152 | HAPLN1, Chr5: 83051152-83054152 |
| NETO1 | Chr18: 68682414-68685414 | NETO1, Chr18: 68682414-68685414 |
| C20orf94 | Chr20: 10362450-10365450 | C20orf94, Chr20: 10362450-10365450 |
| C8orf41 | Chr8: 33488698-33491698 | C8orf41, Chr8: 33488698-33491698 |
| C8orf41 | Chr8: 33488745-33491745 | C8orf41, Chr8: 33488745-33491745 |
| TYW1 | Chr7: 66097751-66100751 | TYW1, Chr7: 66097751-66100751 |
| HIST1H2BH | Chr6: 26358357-26361357 | HIST1H2BH, Chr6: 26358357-26361357 |
| DDT | Chr22: 22650519-22653519 | DDT, Chr22: 22650519-22653519 |
| GOLSYN | Chr8: 110723095-110726095 | GOLSYN, Chr8: 110723095-110726095 |
| FOXQ1 | Chr6: 1256174-1259174 | FOXQ1, Chr6: 1256174-1259174 |
| RREB1 | Chr6: 7051762-7054762 | RREB1, Chr6: 7051762-7054762 |
| WNT11 | Chr11: 75593722-75596722 | WNT11, Chr11: 75593722-75596722 |
| HOXA9 | Chr7: 27170174-27173174 | HOXA9, Chr7: 27170174-27173174 |
| LONRF3 | Chr23: 117991240-117994240 | LONRF3, Chr23: 117991240-117994240 |
| BCL11B | Chr14: 98806075-98809075 | BCL11B, Chr14: 98806075-98809075 |
| SUV420H1 | Chr11: 67735860-67738860 | SUV420H1, Chr11: 67735860-67738860 |
| LRRFIP1 | Chr2: 238264167-238267167 | LRRFIP1, Chr2: 238264167-238267167 |
| BHLHB3 | Chr12: 26167613-26170613 | BHLHB3, Chr12: 26167613-26170613 |
| KCNH5 | Chr14: 62580208-62583208 | KCNH5, Chr14: 62580208-62583208 |
| ACTN3 | Chr11: 66069466-66072466 | ACTN3, Chr11: 66069466-66072466 |
| HIST1H4A | Chr6: 26128385-26131385 | HIST1H4A, Chr6: 26128385-26131385 |
| PURG | Chr8: 31008359-31011359 | PURG, Chr8: 31008359-31011359 |
| HIST1H2AL | Chr6: 27939585-27942585 | HIST1H2AL, Chr6: 27939585-27942585 |
| ZSCAN29 | Chr15: 41448050-41451050 | ZSCAN29, Chr15: 41448050-41451050 |
| GRIN2B | Chr12: 14022789-14025789 | GRIN2B, Chr12: 14022789-14025789 |
| NR3C1 | Chr5: 142762738-142765738 | NR3C1, Chr5: 142762738-142765738 |
| CCDC96 | Chr4: 7094129-7097129 | CCDC96, Chr4: 7094129-7097129 |
| ZBTB16 | Chr11: 113434997-113437997 | ZBTB16, Chr11: 113434997-113437997 |
| CDYL | Chr6: 4720181-4723181 | CDYL, Chr6: 4720181-4723181 |
| SYMPK | Chr19: 51056888-51059888 | SYMPK, Chr19: 51056888-51059888 |
| TRIM72 | Chr16: 31131415-31134415 | TRIM72, Chr16: 31131415-31134415 |
| BNIP3 | Chr10: 133643925-133646925 | BNIP3, Chr10: 133643925-133646925 |
| GABRA2 | Chr4: 46084653-46087653 | GABRA2, Chr4: 46084653-46087653 |
| C20orf46 | Chr20: 1111617-1114617 | C20orf46, Chr20: 1111617-1114617 |
| LRRC32 | Chr11: 76057192-76060192 | LRRC32, Chr11: 76057192-76060192 |
| NEGR1 | Chr1: 72519365-72522365 | NEGR1, Chr1: 72519365-72522365 |
| ZNF235 | Chr19: 49499518-49502518 | ZNF235, Chr19: 49499518-49502518 |
| GAD2 | Chr10: 26544099-26547099 | GAD2, Chr10: 26544099-26547099 |
| GPC4 | Chr23: 132375371-132378371 | GPC4, Chr23: 132375371-132378371 |
| SPTBN1 | Chr2: 54535457-54538457 | SPTBN1, Chr2: 54535457-54538457 |
| CACNA1C | Chr12: 2031176-2034176 | CACNA1C, Chr12: 2031176-2034176 |
| PTTG1IP | Chr21: 45116669-45119669 | PTTG1IP, Chr21: 45116669-45119669 |
| PPARA | Chr22: 44923662-44926662 | PPARA, Chr22: 44923662-44926662 |
| FCHSD2 | Chr11: 72529291-72532291 | FCHSD2, Chr11: 72529291-72532291 |
| B3GALNT2 | Chr1: 233732904-233735904 | B3GALNT2, Chr1: 233732904-233735904 |
| COL4A1 | Chr13: 109755997-109758997 | COL4A1, Chr13: 109755997-109758997 |
| PDE5A | Chr4: 120767150-120770150 | PDE5A, Chr4: 120767150-120770150 |
| ISLR2 | Chr15: 72208497-72211497 | ISLR2, Chr15: 72208497-72211497 |
| ANKS3 | Chr16: 4722664-4725664 | ANKS3, Chr16: 4722664-4725664 |
| GULP1 | Chr2: 188864134-188867134 | GULP1, Chr2: 188864134-188867134 |
| CACNG8 | Chr19: 59156605-59159605 | CACNG8, Chr19: 59156605-59159605 |
| ZNF7 | Chr8: 146022206-146025206 | ZNF7, Chr8: 146022206-146025206 |
| LITAF | Chr16: 11586230-11589230 | LITAF, Chr16: 11586230-11589230 |
| C14orf119 | Chr14: 22633127-22636127 | C14orf119, Chr14: 22633127-22636127 |
| NLGN1 | Chr3: 174597437-174600437 | NLGN1, Chr3: 174597437-174600437 |
| GFI1 | Chr1: 92722716-92725716 | GFI1, Chr1: 92722716-92725716 |
| MEIS2 | Chr15: 35179292-35182292 | MEIS2, Chr15: 35179292-35182292 |
| LOC255374 | Chr7: 99560755-99563755 | LOC255374, Chr7: 99560755-99563755 |
| FAM155B | Chr23: 68640302-68643302 | FAM155B, Chr23: 68640302-68643302 |
| HSD17B14 | Chr19: 54030246-54033246 | HSD17B14, Chr19: 54030246-54033246 |
| ATP6V1D | Chr14: 66894844-66897844 | ATP6V1D, Chr14: 66894844-66897844 |
| DTYMK | Chr2: 242273400-242276400 | DTYMK, Chr2: 242273400-242276400 |
| C6orf65 | Chr6: 56926231-56929231 | C6orf65, Chr6: 56926231-56929231 |
| EIF2S1 | Chr14: 66895286-66898286 | EIF2S1, Chr14: 66895286-66898286 |
| NKAIN3 | Chr8: 63322554-63325554 | NKAIN3, Chr8: 63322554-63325554 |
| ASH1L | Chr1: 153797448-153800448 | ASH1L, Chr1: 153797448-153800448 |
| HSD11B1L | Chr19: 5630534-5633534 | HSD11B1L, Chr19: 5630534-5633534 |
| HSD11B1L | Chr19: 5630546-5633546 | HSD11B1L, Chr19: 5630546-5633546 |
| HSD11B1L | Chr19: 5630551-5633551 | HSD11B1L, Chr19: 5630551-5633551 |
| HSD11B1L | Chr19: 5630574-5633574 | HSD11B1L, Chr19: 5630574-5633574 |
| CLUAP1 | Chr16: 3489463-3492463 | CLUAP1, Chr16: 3489463-3492463 |
| FAM46B | Chr1: 27210414-27213414 | FAM46B, Chr1: 27210414-27213414 |
| DSN1 | Chr20: 34834063-34837063 | DSN1, Chr20: 34834063-34837063 |
| CCNA1 | Chr13: 35902908-35905908 | CCNA1, Chr13: 35902908-35905908 |
| BOC | Chr3: 114412564-114415564 | BOC, Chr3: 114412564-114415564 |
| KIAA1274 | Chr10: 71907069-71910069 | KIAA1274, Chr10: 71907069-71910069 |
| C2orf15 | Chr2: 99123116-99126116 | C2orf15, Chr2: 99123116-99126116 |
| MARS | Chr12: 56166617-56169617 | MARS, Chr12: 56166617-56169617 |
| FAM133A | Chr23: 92814360-92817360 | FAM133A, Chr23: 92814360-92817360 |
| DNER | Chr2: 230286030-230289030 | DNER, Chr2: 230286030-230289030 |
| PTF1A | Chr10: 23519965-23522965 | PTF1A, Chr10: 23519965-23522965 |
| HIPK2 | Chr7: 139122565-139125565 | HIPK2, Chr7: 139122565-139125565 |
| BRF1 | Chr14: 104836874-104839874 | BRF1, Chr14: 104836874-104839874 |
| LAMP1 | Chr13: 112997969-113000969 | LAMP1, Chr13: 112997969-113000969 |
| PABPC1L2A | Chr23: 72214576-72217576 | PABPC1L2A, Chr23: 72214576-72217576 |
| QSER1 | Chr11: 32869867-32872867 | QSER1, Chr11: 32869867-32872867 |
| SLC35F2 | Chr11: 107233364-107236364 | SLC35F2, Chr11: 107233364-107236364 |
| LASS2 | Chr1: 149212564-149215564 | LASS2, Chr1: 149212564-149215564 |
| PLEKHA4 | Chr19: 54062170-54065170 | PLEKHA4, Chr19: 54062170-54065170 |
| SND1 | Chr7: 127077937-127080937 | SND1, Chr7: 127077937-127080937 |
| ZNF580 | Chr19: 60842703-60845703 | ZNF580, Chr19: 60842703-60845703 |
| CABC1 | Chr1: 225193060-225196060 | CABC1, Chr1: 225193060-225196060 |
| IRX6 | Chr16: 53914471-53917471 | IRX6, Chr16: 53914471-53917471 |
| SLC24A3 | Chr20: 19139789-19142789 | SLC24A3, Chr20: 19139789-19142789 |
| CDK8 | Chr13: 25725255-25728255 | CDK8, Chr13: 25725255-25728255 |
| DLX4 | Chr17: 45400062-45403062 | DLX4, Chr17: 45400062-45403062 |
| DACH2 | Chr23: 85288780-85291780 | DACH2, Chr23: 85288780-85291780 |
| ACVRL1 | Chr12: 50585968-50588968 | ACVRL1, Chr12: 50585968-50588968 |
| MLPH | Chr2: 238059116-238062116 | MLPH, Chr2: 238059116-238062116 |
| ZIK1 | Chr19: 62785939-62788939 | ZIK1, Chr19: 62785939-62788939 |
| MT1M | Chr16: 55222534-55225534 | MT1M, Chr16: 55222534-55225534 |
| NOVA1 | Chr14: 26135300-26138300 | NOVA1, Chr14: 26135300-26138300 |
| HSPC111 | Chr5: 175746646-175749646 | HSPC111, Chr5: 175746646-175749646 |
| PPP2R2B | Chr5: 146236852-146239852 | PPP2R2B, Chr5: 146236852-146239852 |
| TANC1 | Chr2: 159531891-159534891 | TANC1, Chr2: 159531891-159534891 |
| INSM2 | Chr14: 35071498-35074498 | INSM2, Chr14: 35071498-35074498 |
| GALNTL1 | Chr14: 68795167-68798167 | GALNTL1, Chr14: 68795167-68798167 |
| CHID1 | Chr11: 899323-902323 | CHID1, Chr11: 899323-902323 |
| DPYSL5 | Chr2: 26922972-26925972 | DPYSL5, Chr2: 26922972-26925972 |
| CDH8 | Chr16: 60626037-60629037 | CDH8, Chr16: 60626037-60629037 |
| BTF3 | Chr5: 72828505-72831505 | BTF3, Chr5: 72828505-72831505 |
| NPHP4 | Chr1: 5973618-5976618 | NPHP4, Chr1: 5973618-5976618 |
| EBF4 | Chr20: 2620023-2623023 | EBF4, Chr20: 2620023-2623023 |
| NRG1 | Chr8: 31615309-31618309 | NRG1, Chr8: 31615309-31618309 |
| SEPX1 | Chr16: 1931795-1934795 | SEPX1, Chr16: 1931795-1934795 |
| COL4A2 | Chr13: 109756131-109759131 | COL4A2, Chr13: 109756131-109759131 |
| SLC5A5 | Chr19: 17842281-17845281 | SLC5A5, Chr19: 17842281-17845281 |
| SPIRE1 | Chr18: 12646412-12649412 | SPIRE1, Chr18: 12646412-12649412 |
| 11-Mar | Chr5: 16231397-16234397 | 40613, Chr5: 16231397-16234397 |
| LRFN2 | Chr6: 40661604-40664604 | LRFN2, Chr6: 40661604-40664604 |
| PTPN14 | Chr1: 212789765-212792765 | PTPN14, Chr1: 212789765-212792765 |
| ZDHHC24 | Chr11: 66068747-66071747 | ZDHHC24, Chr11: 66068747-66071747 |
| ASXL3 | Chr18: 29411038-29414038 | ASXL3, Chr18: 29411038-29414038 |
| RAD52 | Chr12: 927624-930624 | RAD52, Chr12: 927624-930624 |
| NUPL1 | Chr13: 24772165-24775165 | NUPL1, Chr13: 24772165-24775165 |
| FRG1 | Chr4: 191097467-191100467 | FRG1, Chr4: 191097467-191100467 |
| MANEAL | Chr1: 38030860-38033860 | MANEAL, Chr1: 38030860-38033860 |
| CRYBA2 | Chr2: 219564865-219567865 | CRYBA2, Chr2: 219564865-219567865 |
| CRYBA2 | Chr2: 219564871-219567871 | CRYBA2, Chr2: 219564871-219567871 |
| ATP1B1 | Chr1: 167341070-167344070 | ATP1B1, Chr1: 167341070-167344070 |
| PRMT7 | Chr16: 66900945-66903945 | PRMT7, Chr16: 66900945-66903945 |
| BMP2 | Chr20: 6695244-6698244 | BMP2, Chr20: 6695244-6698244 |
| MAPK14 | Chr6: 36102050-36105050 | MAPK14, Chr6: 36102050-36105050 |
| HOOK2 | Chr19: 12745934-12748934 | HOOK2, Chr19: 12745934-12748934 |
| PGRMC2 | Chr4: 129426898-129429898 | PGRMC2, Chr4: 129426898-129429898 |
| PHYHIPL | Chr10: 60604852-60607852 | PHYHIPL, Chr10: 60604852-60607852 |
| EFEMP1 | Chr2: 56002360-56005360 | EFEMP1, Chr2: 56002360-56005360 |
| PRKG1 | Chr10: 52502739-52505739 | PRKG1, Chr10: 52502739-52505739 |
| A26C1A | Chr2: 131690893-131693893 | A26C1A, Chr2: 131690893-131693893 |
| GNG4 | Chr1: 233878416-233881416 | GNG4, Chr1: 233878416-233881416 |
| CKB | Chr14: 103057423-103060423 | CKB, Chr14: 103057423-103060423 |
| FLT1 | Chr13: 27965765-27968765 | FLT1, Chr13: 27965765-27968765 |
| SLC2A4 | Chr17: 7124277-7127277 | SLC2A4, Chr17: 7124277-7127277 |
| ECHDC2 | Chr1: 53158534-53161534 | ECHDC2, Chr1: 53158534-53161534 |
| SMARCA2 | Chr9: 2003841-2006841 | SMARCA2, Chr9: 2003841-2006841 |
| INTS6 | Chr13: 50923776-50926776 | INTS6, Chr13: 50923776-50926776 |
| KLF4 | Chr9: 109290368-109293368 | KLF4, Chr9: 109290368-109293368 |
| TOX3 | Chr16: 51136807-51139807 | TOX3, Chr16: 51136807-51139807 |
| TIPARP | Chr3: 157873572-157876572 | TIPARP, Chr3: 157873572-157876572 |
| MAL | Chr2: 95053705-95056705 | MAL, Chr2: 95053705-95056705 |
| STAU2 | Chr8: 74820111-74823111 | STAU2, Chr8: 74820111-74823111 |
| SERBP1 | Chr1: 67667211-67670211 | SERBP1, Chr1: 67667211-67670211 |
| C1QL1 | Chr17: 40399670-40402670 | C1QL1, Chr17: 40399670-40402670 |
| ZFPM2 | Chr8: 106398822-106401822 | ZFPM2, Chr8: 106398822-106401822 |
| ZNF496 | Chr1: 245560168-245563168 | ZNF496, Chr1: 245560168-245563168 |
| ASXL1 | Chr20: 30408313-30411313 | ASXL1, Chr20: 30408313-30411313 |
| P117 | Chr19: 5630411-5633411 | P117, Chr19: 5630411-5633411 |
| TMEFF2 | Chr2: 192766389-192769389 | TMEFF2, Chr2: 192766389-192769389 |
| MAPK8IP2 | Chr22: 49384496-49387496 | MAPK8IP2, Chr22: 49384496-49387496 |
| MAL2 | Chr8: 120288290-120291290 | MAL2, Chr8: 120288290-120291290 |
| PPIL6 | Chr6: 109867040-109870040 | PPIL6, Chr6: 109867040-109870040 |
| PCMT1 | Chr6: 150111157-150114157 | PCMT1, Chr6: 150111157-150114157 |
| FAM24B | Chr10: 124627647-124630647 | FAM24B, Chr10: 124627647-124630647 |
| GLB1L3 | Chr11: 133649984-133652984 | GLB1L3, Chr11: 133649984-133652984 |
| CNTNAP5 | Chr2: 124497833-124500833 | CNTNAP5, Chr2: 124497833-124500833 |
| NFKB2 | Chr10: 104142828-104145828 | NFKB2, Chr10: 104142828-104145828 |
| OSBPL9 | Chr1: 51966610-51969610 | OSBPL9, Chr1: 51966610-51969610 |
| CACNB3 | Chr12: 47497278-47500278 | CACNB3, Chr12: 47497278-47500278 |
| SUV420H2 | Chr19: 60541532-60544532 | SUV420H2, Chr19: 60541532-60544532 |
| MTERFD1 | Chr8: 97341472-97344472 | MTERFD1, Chr8: 97341472-97344472 |
| PPME1 | Chr11: 73558515-73561515 | PPME1, Chr11: 73558515-73561515 |
| FAM159A | Chr1: 52870153-52873153 | FAM159A, Chr1: 52870153-52873153 |
| DPY19L2 | Chr12: 62347121-62350121 | DPY19L2, Chr12: 62347121-62350121 |
| NPAT | Chr11: 107597075-107600075 | NPAT, Chr11: 107597075-107600075 |
| KCNJ10 | Chr1: 158305085-158308085 | KCNJ10, Chr1: 158305085-158308085 |
| PEX5L | Chr3: 181235711-181238711 | PEX5L, Chr3: 181235711-181238711 |
| TMEM16E | Chr11: 22169797-22172797 | TMEM16E, Chr11: 22169797-22172797 |
| CHRDL2 | Chr11: 74118334-74121334 | CHRDL2, Chr11: 74118334-74121334 |
| LRRC20 | Chr10: 71809920-71812920 | LRRC20, Chr10: 71809920-71812920 |
| PPP2R2B | Chr5: 146237020-146240020 | PPP2R2B, Chr5: 146237020-146240020 |
| CNIH3 | Chr1: 222869301-222872301 | CNIH3, Chr1: 222869301-222872301 |
| RNF122 | Chr8: 33542685-33545685 | RNF122, Chr8: 33542685-33545685 |
| COBL | Chr7: 51350509-51353509 | COBL, Chr7: 51350509-51353509 |
| PLD5 | Chr1: 240753121-240756121 | PLD5, Chr1: 240753121-240756121 |
| ZADH2 | Chr18: 71048605-71051605 | ZADH2, Chr18: 71048605-71051605 |
| MAP6 | Chr11: 75055627-75058627 | MAP6, Chr11: 75055627-75058627 |
| CBLN4 | Chr20: 54011919-54014919 | CBLN4, Chr20: 54011919-54014919 |
| NRG1 | Chr8: 32523794-32526794 | NRG1, Chr8: 32523794-32526794 |
| TMEFF1 | Chr9: 102274037-102277037 | TMEFF1, Chr9: 102274037-102277037 |
| RNF139 | Chr8: 125554688-125557688 | RNF139, Chr8: 125554688-125557688 |
| GALNT13 | Chr2: 154435171-154438171 | GALNT13, Chr2: 154435171-154438171 |
| PLEKHO1 | Chr1: 148387293-148390293 | PLEKHO1, Chr1: 148387293-148390293 |
| NRXN2 | Chr11: 64165863-64168863 | NRXN2, Chr11: 64165863-64168863 |
| ELOVL2 | Chr6: 11151110-11154110 | ELOVL2, Chr6: 11151110-11154110 |
| SLCO3A1 | Chr15: 90196449-90199449 | SLCO3A1, Chr15: 90196449-90199449 |
| NELL1 | Chr11: 20646211-20649211 | NELL1, Chr11: 20646211-20649211 |
| LSM14A | Chr19: 39353691-39356691 | LSM14A, Chr19: 39353691-39356691 |
| CRTAC1 | Chr10: 99779075-99782075 | CRTAC1, Chr10: 99779075-99782075 |
| ACAT2 | Chr6: 160101478-160104478 | ACAT2, Chr6: 160101478-160104478 |
| RPL18A | Chr19: 17830230-17833230 | RPL18A, Chr19: 17830230-17833230 |
| CFTR | Chr7: 116905752-116908752 | CFTR, Chr7: 116905752-116908752 |
| SMPD2 | Chr6: 109867123-109870123 | SMPD2, Chr6: 109867123-109870123 |
| NFKB2 | Chr10: 104142718-104145718 | NFKB2, Chr10: 104142718-104145718 |
| DPPA5 | Chr6: 74119174-74122174 | DPPA5, Chr6: 74119174-74122174 |
| DEPDC2 | Chr8: 69025656-69028656 | DEPDC2, Chr8: 69025656-69028656 |
| ISL1 | Chr5: 50713214-50716214 | ISL1, Chr5: 50713214-50716214 |
| NXPH2 | Chr2: 139252781-139255781 | NXPH2, Chr2: 139252781-139255781 |
| MBD4 | Chr3: 130640042-130643042 | MBD4, Chr3: 130640042-130643042 |
| C2CD3 | Chr11: 73558212-73561212 | C2CD3, Chr11: 73558212-73561212 |
| FLJ14154 | Chr16: 3432168-3435168 | FLJ14154, Chr16: 3432168-3435168 |
| ZNF597 | Chr16: 3431991-3434991 | ZNF597, Chr16: 3431991-3434991 |
| TMEM130 | Chr7: 98304073-98307073 | TMEM130, Chr7: 98304073-98307073 |
| 3-Sep | Chr22: 40701376-40704376 | 40789, Chr22: 40701376-40704376 |
| OSBPL9 | Chr1: 51966580-51969580 | OSBPL9, Chr1: 51966580-51969580 |
| PTDSS1 | Chr8: 97341842-97344842 | PTDSS1, Chr8: 97341842-97344842 |
| PPP2R2B | Chr5: 146237150-146240150 | PPP2R2B, Chr5: 146237150-146240150 |
| SEMA4C | Chr2: 96897962-96900962 | SEMA4C, Chr2: 96897962-96900962 |
| D4S234E | Chr4: 4437383-4440383 | D4S234E, Chr4: 4437383-4440383 |
| IGSF9B | Chr11: 133330590-133333590 | IGSF9B, Chr11: 133330590-133333590 |
| DMN | Chr15: 97461308-97464308 | DMN, Chr15: 97461308-97464308 |
| PTPRJ | Chr11: 47957185-47960185 | PTPRJ, Chr11: 47957185-47960185 |
| LAPTM4B | Chr8: 98855484-98858484 | LAPTM4B, Chr8: 98855484-98858484 |
| TCFL5 | Chr20: 60962060-60965060 | TCFL5, Chr20: 60962060-60965060 |
| ARHGEF1 | Chr19: 47077606-47080606 | ARHGEF1, Chr19: 47077606-47080606 |
| CUGBP2 | Chr10: 11098398-11101398 | CUGBP2, Chr10: 11098398-11101398 |
| FNDC1 | Chr6: 159508916-159511916 | FNDC1, Chr6: 159508916-159511916 |
| KCNK1 | Chr1: 231814872-231817872 | KCNK1, Chr1: 231814872-231817872 |
| CALM2 | Chr2: 47255654-47258654 | CALM2, Chr2: 47255654-47258654 |
| C16orf71 | Chr16: 4722789-4725789 | C16orf71, Chr16: 4722789-4725789 |
| FBXO32 | Chr8: 124621127-124624127 | FBXO32, Chr8: 124621127-124624127 |
| MGC42105 | Chr5: 43226583-43229583 | MGC42105, Chr5: 43226583-43229583 |
| GABRA2 | Chr4: 46085313-46088313 | GABRA2, Chr4: 46085313-46088313 |
| FABP5 | Chr8: 82353839-82356839 | FABP5, Chr8: 82353839-82356839 |
| VEGFC | Chr4: 177949389-177952389 | VEGFC, Chr4: 177949389-177952389 |
| RFWD2 | Chr1: 174441493-174444493 | RFWD2, Chr1: 174441493-174444493 |
| CCNA1 | Chr13: 35902466-35905466 | CCNA1, Chr13: 35902466-35905466 |
| CADM4 | Chr19: 48834331-48837331 | CADM4, Chr19: 48834331-48837331 |
| MALT1 | Chr18: 54488097-54491097 | MALT1, Chr18: 54488097-54491097 |
| HCK | Chr20: 30102217-30105217 | HCK, Chr20: 30102217-30105217 |
| SORCS1 | Chr10: 108912782-108915782 | SORCS1, Chr10: 108912782-108915782 |
| GSG2 | Chr17: 3572445-3575445 | GSG2, Chr17: 3572445-3575445 |
| C12orf44 | Chr12: 50748524-50751524 | C12orf44, Chr12: 50748524-50751524 |
| MIXL1 | Chr1: 224476505-224479505 | MIXL1, Chr1: 224476505-224479505 |
| MTFR1 | Chr8: 66718027-66721027 | MTFR1, Chr8: 66718027-66721027 |
| IFT122 | Chr3: 130640157-130643157 | IFT122, Chr3: 130640157-130643157 |
| SLC16A12 | Chr10: 91283793-91286793 | SLC16A12, Chr10: 91283793-91286793 |
| LRRC32 | Chr11: 76057939-76060939 | LRRC32, Chr11: 76057939-76060939 |
| MMP16 | Chr8: 89407333-89410333 | MMP16, Chr8: 89407333-89410333 |
| MGC13057 | Chr2: 190752333-190755333 | MGC13057, Chr2: 190752333-190755333 |
| ATXN7L1 | Chr7: 105302767-105305767 | ATXN7L1, Chr7: 105302767-105305767 |
| POLM | Chr7: 44087154-44090154 | POLM, Chr7: 44087154-44090154 |
| MYCBPAP | Chr17: 45939310-45942310 | MYCBPAP, Chr17: 45939310-45942310 |
| COX7A2L | Chr2: 42440360-42443360 | COX7A2L, Chr2: 42440360-42443360 |
| EIF2B2 | Chr14: 74537864-74540864 | EIF2B2, Chr14: 74537864-74540864 |
| INPP4B | Chr4: 143985554-143988554 | INPP4B, Chr4: 143985554-143988554 |
| MGC13057 | Chr2: 190752425-190755425 | MGC13057, Chr2: 190752425-190755425 |
| ARL6 | Chr3: 98964784-98967784 | ARL6, Chr3: 98964784-98967784 |
| GPR83 | Chr11: 93772566-93775566 | GPR83, Chr11: 93772566-93775566 |
| STARD3NL | Chr7: 38182957-38185957 | STARD3NL, Chr7: 38182957-38185957 |
| PDE4B | Chr1: 66029943-66032943 | PDE4B, Chr1: 66029943-66032943 |
| SOX3 | Chr23: 139413391-139416391 | SOX3, Chr23: 139413391-139416391 |
| VWC2 | Chr7: 49782302-49785302 | VWC2, Chr7: 49782302-49785302 |
| ADAMTS2 | Chr5: 178703435-178706435 | ADAMTS2, Chr5: 178703435-178706435 |
| DMRT1 | Chr9: 830189-833189 | DMRT1, Chr9: 830189-833189 |
| NRCAM | Chr7: 107882562-107885562 | NRCAM, Chr7: 107882562-107885562 |
| GPX4 | Chr19: 1053435-1056435 | GPX4, Chr19: 1053435-1056435 |
| EMID2 | Chr7: 100791341-100794341 | EMID2, Chr7: 100791341-100794341 |
| SGEF | Chr3: 155320383-155323383 | SGEF, Chr3: 155320383-155323383 |
| LRCH1 | Chr13: 46023803-46026803 | LRCH1, Chr13: 46023803-46026803 |
| ZAR1 | Chr4: 48185565-48188565 | ZAR1, Chr4: 48185565-48188565 |
| TGFA | Chr2: 70633113-70636113 | TGFA, Chr2: 70633113-70636113 |
| PHACTR3 | Chr20: 57611497-57614497 | PHACTR3, Chr20: 57611497-57614497 |
| RAB6B | Chr3: 135095881-135098881 | RAB6B, Chr3: 135095881-135098881 |
| C1orf96 | Chr1: 227543811-227546811 | C1orf96, Chr1: 227543811-227546811 |
| GSX1 | Chr13: 27263279-27266279 | GSX1, Chr13: 27263279-27266279 |
| FMNL2 | Chr2: 152898496-152901496 | FMNL2, Chr2: 152898496-152901496 |
| HCN1 | Chr5: 45730477-45733477 | HCN1, Chr5: 45730477-45733477 |
| HDAC2 | Chr6: 114397547-114400547 | HDAC2, Chr6: 114397547-114400547 |
| PDE5A | Chr4: 120767929-120770929 | PDE5A, Chr4: 120767929-120770929 |
| FRMD3 | Chr9: 85341668-85344668 | FRMD3, Chr9: 85341668-85344668 |
| RND2 | Chr17: 38429283-38432283 | RND2, Chr17: 38429283-38432283 |
| JMJD1C | Chr10: 64894228-64897228 | JMJD1C, Chr10: 64894228-64897228 |
| DGCR2 | Chr22: 17488467-17491467 | DGCR2, Chr22: 17488467-17491467 |
| XTP3TPA | Chr16: 30347374-30350374 | XTP3TPA, Chr16: 30347374-30350374 |
| GNG12 | Chr1: 68070230-68073230 | GNG12, Chr1: 68070230-68073230 |
| KCNK3 | Chr2: 26767622-26770622 | KCNK3, Chr2: 26767622-26770622 |
| 4-Mar | Chr2: 216943495-216946495 | 40606, Chr2: 216943495-216946495 |
| TMEM163 | Chr2: 135191540-135194540 | TMEM163, Chr2: 135191540-135194540 |
| C9orf98 | Chr9: 134742519-134745519 | C9orf98, Chr9: 134742519-134745519 |
| WRN | Chr8: 31008819-31011819 | WRN, Chr8: 31008819-31011819 |
| SOX5 | Chr12: 24605147-24608147 | SOX5, Chr12: 24605147-24608147 |
| ADAMTS15 | Chr11: 129822578-129825578 | ADAMTS15, Chr11: 129822578-129825578 |
| A4GALT | Chr22: 41445320-41448320 | A4GALT, Chr22: 41445320-41448320 |
| SLC7A6OS | Chr16: 66900849-66903849 | SLC7A6OS, Chr16: 66900849-66903849 |
| NALCN | Chr13: 100865314-100868314 | NALCN, Chr13: 100865314-100868314 |
| HIGD2A | Chr5: 175746889-175749889 | HIGD2A, Chr5: 175746889-175749889 |
| ADD1 | Chr4: 2813881-2816881 | ADD1, Chr4: 2813881-2816881 |
| DHCR24 | Chr1: 55124009-55127009 | DHCR24, Chr1: 55124009-55127009 |
| FLNB | Chr3: 57967666-57970666 | FLNB, Chr3: 57967666-57970666 |
| ADAM23 | Chr2: 207015112-207018112 | ADAM23, Chr2: 207015112-207018112 |
| ATM | Chr11: 107597268-107600268 | ATM, Chr11: 107597268-107600268 |
| CARS2 | Chr13: 110154964-110157964 | CARS2, Chr13: 110154964-110157964 |
| ABHD10 | Chr3: 113179017-113182017 | ABHD10, Chr3: 113179017-113182017 |
| TDG | Chr12: 102882246-102885246 | TDG, Chr12: 102882246-102885246 |
| ABCB4 | Chr7: 86941455-86944455 | ABCB4, Chr7: 86941455-86944455 |
| DKK1 | Chr10: 53742546-53745546 | DKK1, Chr10: 53742546-53745546 |
| MOBKL3 | Chr2: 198087515-198090515 | MOBKL3, Chr2: 198087515-198090515 |
| TSLP | Chr5: 110435424-110438424 | TSLP, Chr5: 110435424-110438424 |
| TUBGCP4 | Chr15: 41449104-41452104 | TUBGCP4, Chr15: 41449104-41452104 |
| LOC200726 | Chr2: 207213886-207216886 | LOC200726, Chr2: 207213886-207216886 |
| VSNL1 | Chr2: 17583787-17586787 | VSNL1, Chr2: 17583787-17586787 |
| HTR1E | Chr6: 87702628-87705628 | HTR1E, Chr6: 87702628-87705628 |
| GINS4 | Chr8: 41504402-41507402 | GINS4, Chr8: 41504402-41507402 |
| C2orf65 | Chr2: 74727172-74730172 | C2orf65, Chr2: 74727172-74730172 |
| CENPQ | Chr6: 49537554-49540554 | CENPQ, Chr6: 49537554-49540554 |
| VSTM2A | Chr7: 54576012-54579012 | VSTM2A, Chr7: 54576012-54579012 |
| PTH2R | Chr2: 208978300-208981300 | PTH2R, Chr2: 208978300-208981300 |
| ZBTB16 | Chr11: 113434140-113437140 | ZBTB16, Chr11: 113434140-113437140 |
| KIFC1 | Chr6: 33465790-33468790 | KIFC1, Chr6: 33465790-33468790 |
| FAM78B | Chr1: 164401330-164404330 | FAM78B, Chr1: 164401330-164404330 |
| FAM19A4 | Chr3: 69062901-69065901 | FAM19A4, Chr3: 69062901-69065901 |
| TTC32 | Chr2: 19963725-19966725 | TTC32, Chr2: 19963725-19966725 |
| PKP4 | Chr2: 159020221-159023221 | PKP4, Chr2: 159020221-159023221 |
| TMPRSS2 | Chr21: 41800448-41803448 | TMPRSS2, Chr21: 41800448-41803448 |
| STOX2 | Chr4: 185062002-185065002 | STOX2, Chr4: 185062002-185065002 |
| PCSK5 | Chr9: 77693940-77696940 | PCSK5, Chr9: 77693940-77696940 |
| SPATA18 | Chr4: 52610849-52613849 | SPATA18, Chr4: 52610849-52613849 |
| SLITRK3 | Chr3: 166395663-166398663 | SLITRK3, Chr3: 166395663-166398663 |
| KIAA0888 | Chr5: 74196871-74199871 | KIAA0888, Chr5: 74196871-74199871 |
| SNX11 | Chr17: 43538445-43541445 | SNX11, Chr17: 43538445-43541445 |
| SNX11 | Chr17: 43538418-43541418 | SNX11, Chr17: 43538418-43541418 |
| PNPLA3 | Chr22: 42649451-42652451 | PNPLA3, Chr22: 42649451-42652451 |
| PRMT6 | Chr1: 107399431-107402431 | PRMT6, Chr1: 107399431-107402431 |
| A2BP1 | Chr16: 6007632-6010632 | A2BP1, Chr16: 6007632-6010632 |
| POMC | Chr2: 25243563-25246563 | POMC, Chr2: 25243563-25246563 |
| RNF144A | Chr2: 6973473-6976473 | RNF144A, Chr2: 6973473-6976473 |
| NEIL1 | Chr15: 73424962-73427962 | NEIL1, Chr15: 73424962-73427962 |
| LIN7B | Chr19: 54307929-54310929 | LIN7B, Chr19: 54307929-54310929 |
| RSPO1 | Chr1: 37871578-37874578 | RSPO1, Chr1: 37871578-37874578 |
| LDLRAD3 | Chr11: 35920687-35923687 | LDLRAD3, Chr11: 35920687-35923687 |
| LYST | Chr1: 234095343-234098343 | LYST, Chr1: 234095343-234098343 |
| IGFBP7 | Chr4: 57669796-57672796 | IGFBP7, Chr4: 57669796-57672796 |
| SYN2 | Chr3: 12019361-12022361 | SYN2, Chr3: 12019361-12022361 |
| PURG | Chr8: 31009273-31012273 | PURG, Chr8: 31009273-31012273 |
| LRCH3 | Chr3: 199001041-199004041 | LRCH3, Chr3: 199001041-199004041 |
| FKBP1B | Chr2: 24124631-24127631 | FKBP1B, Chr2: 24124631-24127631 |
| NCK1 | Chr3: 138062262-138065262 | NCK1, Chr3: 138062262-138065262 |
| TMEM155 | Chr4: 122904290-122907290 | TMEM155, Chr4: 122904290-122907290 |
| EIF6 | Chr20: 33334420-33337420 | EIF6, Chr20: 33334420-33337420 |
| BRUNOL6 | Chr15: 70398079-70401079 | BRUNOL6, Chr15: 70398079-70401079 |
| ACOXL | Chr2: 111205179-111208179 | ACOXL, Chr2: 111205179-111208179 |
| FZD4 | Chr11: 86342581-86345581 | FZD4, Chr11: 86342581-86345581 |
| ENTPD4 | Chr8: 23369581-23372581 | ENTPD4, Chr8: 23369581-23372581 |
| PI4K2A | Chr10: 99388932-99391932 | PI4K2A, Chr10: 99388932-99391932 |
| TXNL1 | Chr18: 52455374-52458374 | TXNL1, Chr18: 52455374-52458374 |
| SLC6A20 | Chr3: 45811466-45814466 | SLC6A20, Chr3: 45811466-45814466 |
| DHX40 | Chr17: 54996167-54999167 | DHX40, Chr17: 54996167-54999167 |
| PTER | Chr10: 16517472-16520472 | PTER, Chr10: 16517472-16520472 |
| DUSP12 | Chr1: 159984704-159987704 | DUSP12, Chr1: 159984704-159987704 |
| UNQ9438 | Chr14: 57930896-57933896 | UNQ9438, Chr14: 57930896-57933896 |
| C7orf31 | Chr7: 25184842-25187842 | C7orf31, Chr7: 25184842-25187842 |
| KIAA1853 | Chr12: 117902278-117905278 | KIAA1853, Chr12: 117902278-117905278 |
| C2orf40 | Chr2: 106047044-106050044 | C2orf40, Chr2: 106047044-106050044 |
| SEC22A | Chr3: 124401964-124404964 | SEC22A, Chr3: 124401964-124404964 |
| MRPS16 | Chr10: 74680957-74683957 | MRPS16, Chr10: 74680957-74683957 |
| GABBR1 | Chr6: 29702484-29705484 | GABBR1, Chr6: 29702484-29705484 |
| SLC35E3 | Chr12: 67424702-67427702 | SLC35E3, Chr12: 67424702-67427702 |
| IRAK3 | Chr12: 64867783-64870783 | IRAK3, Chr12: 64867783-64870783 |
| LOC440585 | Chr1: 43384680-43387680 | LOC440585, Chr1: 43384680-43387680 |
| GRM2 | Chr3: 51714620-51717620 | GRM2, Chr3: 51714620-51717620 |
| PTPLAD2 | Chr9: 21020135-21023135 | PTPLAD2, Chr9: 21020135-21023135 |
| ZMYND10 | Chr3: 50356660-50359660 | ZMYND10, Chr3: 50356660-50359660 |
| CCDC39 | Chr3: 181878477-181881477 | CCDC39, Chr3: 181878477-181881477 |
| C20orf4 | Chr20: 34286360-34289360 | C20orf4, Chr20: 34286360-34289360 |
| WDR17 | Chr4: 177222625-177225625 | WDR17, Chr4: 177222625-177225625 |
| ERLIN1 | Chr10: 101934224-101937224 | ERLIN1, Chr10: 101934224-101937224 |
| ELMO1 | Chr7: 37453536-37456536 | ELMO1, Chr7: 37453536-37456536 |
| GFI1 | Chr1: 92723521-92726521 | GFI1, Chr1: 92723521-92726521 |
| CALY | Chr10: 134998965-135001965 | CALY, Chr10: 134998965-135001965 |
| PITX2 | Chr4: 111762203-111765203 | PITX2, Chr4: 111762203-111765203 |
| HOXC11 | Chr12: 52651676-52654676 | HOXC11, Chr12: 52651676-52654676 |
| AMOTL1 | Chr11: 94139655-94142655 | AMOTL1, Chr11: 94139655-94142655 |
| GRM7 | Chr3: 6876426-6879426 | GRM7, Chr3: 6876426-6879426 |
| NPNT | Chr4: 107034553-107037553 | NPNT, Chr4: 107034553-107037553 |
| KIAA2022 | Chr23: 74060209-74063209 | KIAA2022, Chr23: 74060209-74063209 |
| SMOX | Chr20: 4075949-4078949 | SMOX, Chr20: 4075949-4078949 |
| FIGLA | Chr2: 70869783-70872783 | FIGLA, Chr2: 70869783-70872783 |
| AFF2 | Chr23: 147388330-147391330 | AFF2, Chr23: 147388330-147391330 |
| RPRM | Chr2: 154042068-154045068 | RPRM, Chr2: 154042068-154045068 |
| C9orf40 | Chr9: 76756122-76759122 | C9orf40, Chr9: 76756122-76759122 |
| RBP7 | Chr1: 9978360-9981360 | RBP7, Chr1: 9978360-9981360 |
| MSC | Chr8: 72917785-72920785 | MSC, Chr8: 72917785-72920785 |
| MYO3A | Chr10: 26261507-26264507 | MYO3A, Chr10: 26261507-26264507 |
| UGT8 | Chr4: 115737559-115740559 | UGT8, Chr4: 115737559-115740559 |
| TSGA10 | Chr2: 99122969-99125969 | TSGA10, Chr2: 99122969-99125969 |
| RPGR | Chr23: 38070232-38073232 | RPGR, Chr23: 38070232-38073232 |
| RIF1 | Chr2: 151973173-151976173 | RIF1, Chr2: 151973173-151976173 |
| HSPA5 | Chr9: 127041982-127044982 | HSPA5, Chr9: 127041982-127044982 |
| TSPYL5 | Chr8: 98357852-98360852 | TSPYL5, Chr8: 98357852-98360852 |
| CDH23 | Chr10: 72825209-72828209 | CDH23, Chr10: 72825209-72828209 |
| TRAF6 | Chr11: 36486898-36489898 | TRAF6, Chr11: 36486898-36489898 |
| DNAJC1 | Chr10: 22331156-22334156 | DNAJC1, Chr10: 22331156-22334156 |
| EFEMP1 | Chr2: 56002936-56005936 | EFEMP1, Chr2: 56002936-56005936 |
| LDOC1 | Chr23: 140097476-140100476 | LDOC1, Chr23: 140097476-140100476 |
| ACADM | Chr1: 75961130-75964130 | ACADM, Chr1: 75961130-75964130 |
| EIF6 | Chr20: 33334508-33337508 | EIF6, Chr20: 33334508-33337508 |
| ZSCAN22 | Chr19: 63528696-63531696 | ZSCAN22, Chr19: 63528696-63531696 |
| ENTPD4 | Chr8: 23369689-23372689 | ENTPD4, Chr8: 23369689-23372689 |
| SLC2A10 | Chr20: 44770185-44773185 | SLC2A10, Chr20: 44770185-44773185 |
| TEKT3 | Chr17: 15184124-15187124 | TEKT3, Chr17: 15184124-15187124 |
| TXNDC5 | Chr6: 8008096-8011096 | TXNDC5, Chr6: 8008096-8011096 |
| MAPKBP1 | Chr15: 39852423-39855423 | MAPKBP1, Chr15: 39852423-39855423 |
| GABRB2 | Chr5: 160906208-160909208 | GABRB2, Chr5: 160906208-160909208 |
| STX17 | Chr9: 101707235-101710235 | STX17, Chr9: 101707235-101710235 |
| GNPTAB | Chr12: 100747263-100750263 | GNPTAB, Chr12: 100747263-100750263 |
| ZNF215 | Chr11: 6902729-6905729 | ZNF215, Chr11: 6902729-6905729 |
| TNFRSF8 | Chr1: 12044520-12047520 | TNFRSF8, Chr1: 12044520-12047520 |
| PCDHGB7 | Chr5: 140775965-140778965 | PCDHGB7, Chr5: 140775965-140778965 |
| PAX3 | Chr2: 222870444-222873444 | PAX3, Chr2: 222870444-222873444 |
| RAB37 | Chr17: 70177364-70180364 | RAB37, Chr17: 70177364-70180364 |
| DYSF | Chr2: 71532767-71535767 | DYSF, Chr2: 71532767-71535767 |
| ATP6V1C1 | Chr8: 104100923-104103923 | ATP6V1C1, Chr8: 104100923-104103923 |
| HDHD2 | Chr18: 42929369-42932369 | HDHD2, Chr18: 42929369-42932369 |
| EPHA3 | Chr3: 89237863-89240863 | EPHA3, Chr3: 89237863-89240863 |
| ADRB2 | Chr5: 148184848-148187848 | ADRB2, Chr5: 148184848-148187848 |
| STARD3 | Chr17: 35045437-35048437 | STARD3, Chr17: 35045437-35048437 |
| BCAN | Chr1: 154876863-154879863 | BCAN, Chr1: 154876863-154879863 |
| AK2 | Chr1: 33273558-33276558 | AK2, Chr1: 33273558-33276558 |
| RBM11 | Chr21: 14508836-14511836 | RBM11, Chr21: 14508836-14511836 |
| NAP1L3 | Chr23: 92813764-92816764 | NAP1L3, Chr23: 92813764-92816764 |
| UNC13B | Chr9: 35150488-35153488 | UNC13B, Chr9: 35150488-35153488 |
| FAM3B | Chr21: 41609030-41612030 | FAM3B, Chr21: 41609030-41612030 |
| CDC5L | Chr6: 44461779-44464779 | CDC5L, Chr6: 44461779-44464779 |
| AK2 | Chr1: 33273579-33276579 | AK2, Chr1: 33273579-33276579 |
| ZNF169 | Chr9: 96059898-96062898 | ZNF169, Chr9: 96059898-96062898 |
| GOLSYN | Chr8: 110724727-110727727 | GOLSYN, Chr8: 110724727-110727727 |
| 4-Sep | Chr17: 53962910-53965910 | 40790, Chr17: 53962910-53965910 |
| LSM10 | Chr1: 36634580-36637580 | LSM10, Chr1: 36634580-36637580 |
| TGFB1I1 | Chr16: 31389484-31392484 | TGFB1I1, Chr16: 31389484-31392484 |
| NAT6 | Chr3: 50310216-50313216 | NAT6, Chr3: 50310216-50313216 |
| MUT | Chr6: 49537490-49540490 | MUT, Chr6: 49537490-49540490 |
| VPS33A | Chr12: 121315521-121318521 | VPS33A, Chr12: 121315521-121318521 |
| ACTL6B | Chr7: 100090486-100093486 | ACTL6B, Chr7: 100090486-100093486 |
| ERLIN1 | Chr10: 101934304-101937304 | ERLIN1, Chr10: 101934304-101937304 |
| PWP1 | Chr12: 106602219-106605219 | PWP1, Chr12: 106602219-106605219 |
| AS3MT | Chr10: 104617699-104620699 | AS3MT, Chr10: 104617699-104620699 |
| SKAP1 | Chr17: 43861093-43864093 | SKAP1, Chr17: 43861093-43864093 |
| GABRA4 | Chr4: 46688837-46691837 | GABRA4, Chr4: 46688837-46691837 |
| DTX3L | Chr3: 123764455-123767455 | DTX3L, Chr3: 123764455-123767455 |
| KIRREL2 | Chr19: 41038149-41041149 | KIRREL2, Chr19: 41038149-41041149 |
| SYT11 | Chr1: 154094423-154097423 | SYT11, Chr1: 154094423-154097423 |
| FOXP4 | Chr6: 41620641-41623641 | FOXP4, Chr6: 41620641-41623641 |
| ESR1 | Chr6: 152168646-152171646 | ESR1, Chr6: 152168646-152171646 |
| SSTR1 | Chr14: 37745454-37748454 | SSTR1, Chr14: 37745454-37748454 |
| HIST1H3A | Chr6: 26127196-26130196 | HIST1H3A, Chr6: 26127196-26130196 |
| NBEA | Chr13: 34412955-34415955 | NBEA, Chr13: 34412955-34415955 |
| VCP | Chr9: 35061239-35064239 | VCP, Chr9: 35061239-35064239 |
| HMGN3 | Chr6: 79999674-80002674 | HMGN3, Chr6: 79999674-80002674 |
| MRPL16 | Chr11: 59333421-59336421 | MRPL16, Chr11: 59333421-59336421 |
| CPLX2 | Chr5: 175154715-175157715 | CPLX2, Chr5: 175154715-175157715 |
| FREM2 | Chr13: 38157672-38160672 | FREM2, Chr13: 38157672-38160672 |
| GRM6 | Chr5: 178353230-178356230 | GRM6, Chr5: 178353230-178356230 |
| ZNF283 | Chr19: 49021812-49024812 | ZNF283, Chr19: 49021812-49024812 |
| LHFPL4 | Chr3: 9568986-9571986 | LHFPL4, Chr3: 9568986-9571986 |
| KAT2B | Chr3: 20055027-20058027 | KAT2B, Chr3: 20055027-20058027 |
| GPR103 | Chr4: 122520131-122523131 | GPR103, Chr4: 122520131-122523131 |
| IMPAD1 | Chr8: 58067481-58070481 | IMPAD1, Chr8: 58067481-58070481 |
| USP11 | Chr23: 46975757-46978757 | USP11, Chr23: 46975757-46978757 |
| TBX15 | Chr1: 119332202-119335202 | TBX15, Chr1: 119332202-119335202 |
| TMEM185A | Chr23: 148519875-148522875 | TMEM185A, Chr23: 148519875-148522875 |
| SLC15A3 | Chr11: 60474333-60477333 | SLC15A3, Chr11: 60474333-60477333 |
| CACNG3 | Chr16: 24172876-24175876 | CACNG3, Chr16: 24172876-24175876 |
| HOXB4 | Chr17: 44009242-44012242 | HOXB4, Chr17: 44009242-44012242 |
| SYT17 | Chr16: 19085638-19088638 | SYT17, Chr16: 19085638-19088638 |
| HOXC12 | Chr12: 52633480-52636480 | HOXC12, Chr12: 52633480-52636480 |
| GNPDA2 | Chr4: 44421869-44424869 | GNPDA2, Chr4: 44421869-44424869 |
| MKKS | Chr20: 10361366-10364366 | MKKS, Chr20: 10361366-10364366 |
| TXNDC6 | Chr3: 139529918-139532918 | TXNDC6, Chr3: 139529918-139532918 |
| PARP1 | Chr1: 224660924-224663924 | PARP1, Chr1: 224660924-224663924 |
| MUTED | Chr6: 8008146-8011146 | MUTED, Chr6: 8008146-8011146 |
| ATG5 | Chr6: 106878888-106881888 | ATG5, Chr6: 106878888-106881888 |
| FBP1 | Chr9: 96440244-96443244 | FBP1, Chr9: 96440244-96443244 |
| FLJ30058 | Chr23: 130018396-130021396 | FLJ30058, Chr23: 130018396-130021396 |
| RAB6C | Chr2: 130452204-130455204 | RAB6C, Chr2: 130452204-130455204 |
| PABPC5 | Chr23: 90574752-90577752 | PABPC5, Chr23: 90574752-90577752 |
| SBDS | Chr7: 66096523-66099523 | SBDS, Chr7: 66096523-66099523 |
| C3orf15 | Chr3: 120903058-120906058 | C3orf15, Chr3: 120903058-120906058 |
| LANCL1 | Chr2: 211048176-211051176 | LANCL1, Chr2: 211048176-211051176 |
| FNTB | Chr14: 64521759-64524759 | FNTB, Chr14: 64521759-64524759 |
| JAM3 | Chr11: 133442529-133445529 | JAM3, Chr11: 133442529-133445529 |
| NECAB1 | Chr8: 91871453-91874453 | NECAB1, Chr8: 91871453-91874453 |
| PPP5C | Chr19: 51540633-51543633 | PPP5C, Chr19: 51540633-51543633 |
| MAOB | Chr23: 43625165-43628165 | MAOB, Chr23: 43625165-43628165 |
| TSPAN33 | Chr7: 128570447-128573447 | TSPAN33, Chr7: 128570447-128573447 |
| C7orf52 | Chr7: 100608777-100611777 | C7orf52, Chr7: 100608777-100611777 |
| ETNK1 | Chr12: 22667842-22670842 | ETNK1, Chr12: 22667842-22670842 |
| GNG4 | Chr1: 233879177-233882177 | GNG4, Chr1: 233879177-233882177 |
| C20orf39 | Chr20: 24396334-24399334 | C20orf39, Chr20: 24396334-24399334 |
| NLGN4X | Chr23: 6154388-6157388 | NLGN4X, Chr23: 6154388-6157388 |
| GADD45A | Chr1: 67921970-67924970 | GADD45A, Chr1: 67921970-67924970 |
| TRIM36 | Chr5: 114542642-114545642 | TRIM36, Chr5: 114542642-114545642 |
| CLCF1 | Chr11: 66896282-66899282 | CLCF1, Chr11: 66896282-66899282 |
| HBQ1 | Chr16: 168834-171834 | HBQ1, Chr16: 168834-171834 |
| PDE1C | Chr7: 32076016-32079016 | PDE1C, Chr7: 32076016-32079016 |
| LOC198437 | Chr20: 62184656-62187656 | LOC198437, Chr20: 62184656-62187656 |
| ACIN1 | Chr14: 22633163-22636163 | ACIN1, Chr14: 22633163-22636163 |
| TSPAN12 | Chr7: 120283913-120286913 | TSPAN12, Chr7: 120283913-120286913 |
| CXCL14 | Chr5: 134941368-134944368 | CXCL14, Chr5: 134941368-134944368 |
| SLC30A10 | Chr1: 218167116-218170116 | SLC30A10, Chr1: 218167116-218170116 |
| EED | Chr11: 85631962-85634962 | EED, Chr11: 85631962-85634962 |
| MOBKL3 | Chr2: 198087064-198090064 | MOBKL3, Chr2: 198087064-198090064 |
| ASTN1 | Chr1: 175399147-175402147 | ASTN1, Chr1: 175399147-175402147 |
| KDELR2 | Chr7: 6488874-6491874 | KDELR2, Chr7: 6488874-6491874 |
| FLJ22662 | Chr12: 14610558-14613558 | FLJ22662, Chr12: 14610558-14613558 |
| ENO2 | Chr12: 6892374-6895374 | ENO2, Chr12: 6892374-6895374 |
| CCDC105 | Chr19: 14981055-14984055 | CCDC105, Chr19: 14981055-14984055 |
| DCDC2 | Chr6: 24464759-24467759 | DCDC2, Chr6: 24464759-24467759 |
| ERN2 | Chr16: 23630822-23633822 | ERN2, Chr16: 23630822-23633822 |
| HIST1H3F | Chr6: 26357314-26360314 | HIST1H3F, Chr6: 26357314-26360314 |
| ZNF323 | Chr6: 28410404-28413404 | ZNF323, Chr6: 28410404-28413404 |
| OPRM1 | Chr6: 154400635-154403635 | OPRM1, Chr6: 154400635-154403635 |
| HYAL3 | Chr3: 50310403-50313403 | HYAL3, Chr3: 50310403-50313403 |
| MOS | Chr8: 57187595-57190595 | MOS, Chr8: 57187595-57190595 |
| KCNK10 | Chr14: 87861504-87864504 | KCNK10, Chr14: 87861504-87864504 |
| C1orf92 | Chr1: 155155547-155158547 | C1orf92, Chr1: 155155547-155158547 |
| PXMP4 | Chr20: 31770297-31773297 | PXMP4, Chr20: 31770297-31773297 |
| PARP9 | Chr3: 123764614-123767614 | PARP9, Chr3: 123764614-123767614 |
| TRPC6 | Chr11: 100958369-100961369 | TRPC6, Chr11: 100958369-100961369 |
| CNGA3 | Chr2: 98327549-98330549 | CNGA3, Chr2: 98327549-98330549 |
| FOXE3 | Chr1: 47652830-47655830 | FOXE3, Chr1: 47652830-47655830 |
| CDH13 | Chr16: 81216578-81219578 | CDH13, Chr16: 81216578-81219578 |
| CR1 | Chr1: 205734595-205737595 | CR1, Chr1: 205734595-205737595 |
| KCNV1 | Chr8: 111054635-111057635 | KCNV1, Chr8: 111054635-111057635 |
| CD38 | Chr4: 15387528-15390528 | CD38, Chr4: 15387528-15390528 |
| KIAA1305 | Chr14: 23936331-23939331 | KIAA1305, Chr14: 23936331-23939331 |
| HLA-F | Chr6: 29797595-29800595 | HLA-F, Chr6: 29797595-29800595 |
| EDNRA | Chr4: 148619856-148622856 | EDNRA, Chr4: 148619856-148622856 |
| CD34 | Chr1: 206149806-206152806 | CD34, Chr1: 206149806-206152806 |
| DDX56 | Chr7: 44579162-44582162 | DDX56, Chr7: 44579162-44582162 |
| CHMP4A | Chr14: 23751525-23754525 | CHMP4A, Chr14: 23751525-23754525 |
| ZNF280B | Chr22: 21192005-21195005 | ZNF280B, Chr22: 21192005-21195005 |
| ALDH7A1 | Chr5: 125957481-125960481 | ALDH7A1, Chr5: 125957481-125960481 |
| LRRC20 | Chr10: 71810888-71813888 | LRRC20, Chr10: 71810888-71813888 |
| PCDH8 | Chr13: 52319275-52322275 | PCDH8, Chr13: 52319275-52322275 |
| MARK2 | Chr11: 63411150-63414150 | MARK2, Chr11: 63411150-63414150 |
| NCAM1 | Chr11: 112335704-112338704 | NCAM1, Chr11: 112335704-112338704 |
| NMNAT2 | Chr1: 181652860-181655860 | NMNAT2, Chr1: 181652860-181655860 |
| EPHA10 | Chr1: 38001911-38004911 | EPHA10, Chr1: 38001911-38004911 |
| KAAG1 | Chr6: 24463609-24466609 | KAAG1, Chr6: 24463609-24466609 |
| FLJ41766 | Chr16: 21218170-21221170 | FLJ41766, Chr16: 21218170-21221170 |
| FBP1 | Chr9: 96440852-96443852 | FBP1, Chr9: 96440852-96443852 |
| KRT36 | Chr17: 36898142-36901142 | KRT36, Chr17: 36898142-36901142 |
| C10orf67 | Chr10: 23672278-23675278 | C10orf67, Chr10: 23672278-23675278 |
| CRH | Chr8: 67251752-67254752 | CRH, Chr8: 67251752-67254752 |
| CRHBP | Chr5: 76282935-76285935 | CRHBP, Chr5: 76282935-76285935 |
| GOLSYN | Chr8: 110725498-110728498 | GOLSYN, Chr8: 110725498-110728498 |
| LRP1B | Chr2: 142604240-142607240 | LRP1B, Chr2: 142604240-142607240 |
| PDGFRA | Chr4: 54788520-54791520 | PDGFRA, Chr4: 54788520-54791520 |
| CD1D | Chr1: 156414860-156417860 | CD1D, Chr1: 156414860-156417860 |
| NLGN4X | Chr23: 6155206-6158206 | NLGN4X, Chr23: 6155206-6158206 |
| FOXD4L5 | Chr9: 69467135-69470135 | FOXD4L5, Chr9: 69467135-69470135 |
| MYL3 | Chr3: 46878477-46881477 | MYL3, Chr3: 46878477-46881477 |
| GSTT2 | Chr22: 22650813-22653813 | GSTT2, Chr22: 22650813-22653813 |
| PIF1 | Chr15: 62903391-62906391 | PIF1, Chr15: 62903391-62906391 |
| CRYM | Chr16: 21220418-21223418 | CRYM, Chr16: 21220418-21223418 |
| CREB3L3 | Chr19: 4103128-4106128 | CREB3L3, Chr19: 4103128-4106128 |
| TSLP | Chr5: 110433788-110436788 | TSLP, Chr5: 110433788-110436788 |
| ATP2B4 | Chr1: 201861050-201864050 | ATP2B4, Chr1: 201861050-201864050 |
| UROD | Chr1: 45248916-45251916 | UROD, Chr1: 45248916-45251916 |
| C9orf9 | Chr9: 134742610-134745610 | C9orf9, Chr9: 134742610-134745610 |
| C21orf33 | Chr21: 44376421-44379421 | C21orf33, Chr21: 44376421-44379421 |
| MARK2 | Chr11: 63361970-63364970 | MARK2, Chr11: 63361970-63364970 |
| SGK1 | Chr6: 134536227-134539227 | SGK1, Chr6: 134536227-134539227 |
| GMPPA | Chr2: 220070372-220073372 | GMPPA, Chr2: 220070372-220073372 |
| SLK | Chr10: 105715959-105718959 | SLK, Chr10: 105715959-105718959 |
| ATP11A | Chr13: 112391143-112394143 | ATP11A, Chr13: 112391143-112394143 |
| XRCC1 | Chr19: 48770070-48773070 | XRCC1, Chr19: 48770070-48773070 |
| WNT7B | Chr22: 44749895-44752895 | WNT7B, Chr22: 44749895-44752895 |
| APITD1 | Chr1: 10411886-10414886 | APITD1, Chr1: 10411886-10414886 |
| EML1 | Chr14: 99327997-99330997 | EML1, Chr14: 99327997-99330997 |
| PDPK1 | Chr16: 2526470-2529470 | PDPK1, Chr16: 2526470-2529470 |
| SEMA5A | Chr5: 9597733-9600733 | SEMA5A, Chr5: 9597733-9600733 |
| GBX1 | Chr7: 150494300-150497300 | GBX1, Chr7: 150494300-150497300 |
| FDXR | Chr17: 70379251-70382251 | FDXR, Chr17: 70379251-70382251 |
| TSC2 | Chr16: 2036490-2039490 | TSC2, Chr16: 2036490-2039490 |
| C11orf82 | Chr11: 82288884-82291884 | C11orf82, Chr11: 82288884-82291884 |
| ST8SIA2 | Chr15: 90736643-90739643 | ST8SIA2, Chr15: 90736643-90739643 |
| NPPC | Chr2: 232497703-232500703 | NPPC, Chr2: 232497703-232500703 |
| SHKBP1 | Chr19: 45773129-45776129 | SHKBP1, Chr19: 45773129-45776129 |
| PHF2 | Chr9: 95377229-95380229 | PHF2, Chr9: 95377229-95380229 |
| C20orf29 | Chr20: 3747702-3750702 | C20orf29, Chr20: 3747702-3750702 |
| DOCK11 | Chr23: 117512399-117515399 | DOCK11, Chr23: 117512399-117515399 |
| WDR21A | Chr14: 72461337-72464337 | WDR21A, Chr14: 72461337-72464337 |
| GIPC1 | Chr19: 14466444-14469444 | GIPC1, Chr19: 14466444-14469444 |
| THRA | Chr17: 35471185-35474185 | THRA, Chr17: 35471185-35474185 |
| TCEB1 | Chr8: 75045400-75048400 | TCEB1, Chr8: 75045400-75048400 |
| DHPS | Chr19: 12652177-12655177 | DHPS, Chr19: 12652177-12655177 |
| ATOH8 | Chr2: 85833032-85836032 | ATOH8, Chr2: 85833032-85836032 |
| CDC42EP1 | Chr22: 36284916-36287916 | CDC42EP1, Chr22: 36284916-36287916 |
| ZC3HAV1 | Chr7: 138443505-138446505 | ZC3HAV1, Chr7: 138443505-138446505 |
| PURA | Chr5: 139472391-139475391 | PURA, Chr5: 139472391-139475391 |
| CRMP1 | Chr4: 5939716-5942716 | CRMP1, Chr4: 5939716-5942716 |
| RTEL1 | Chr20: 61758590-61761590 | RTEL1, Chr20: 61758590-61761590 |
| NBPF3 | Chr1: 21637717-21640717 | NBPF3, Chr1: 21637717-21640717 |
| CEP72 | Chr5: 663904-666904 | CEP72, Chr5: 663904-666904 |
| ZNF181 | Chr19: 39915432-39918432 | ZNF181, Chr19: 39915432-39918432 |
| MEF2C | Chr5: 88213280-88216280 | MEF2C, Chr5: 88213280-88216280 |
| EGFR | Chr7: 55052718-55055718 | EGFR, Chr7: 55052718-55055718 |
| HNRNPAB | Chr5: 177562613-177565613 | HNRNPAB, Chr5: 177562613-177565613 |
| CD81 | Chr11: 2353622-2356622 | CD81, Chr11: 2353622-2356622 |
| FKBP9 | Chr7: 32962076-32965076 | FKBP9, Chr7: 32962076-32965076 |
| SESN3 | Chr11: 94602394-94605394 | SESN3, Chr11: 94602394-94605394 |
| CPD | Chr17: 25728609-25731609 | CPD, Chr17: 25728609-25731609 |
| ARPC4 | Chr3: 9807726-9810726 | ARPC4, Chr3: 9807726-9810726 |
| CD55 | Chr1: 205559939-205562939 | CD55, Chr1: 205559939-205562939 |
| RUNDC3B | Chr7: 87094164-87097164 | RUNDC3B, Chr7: 87094164-87097164 |
| ZNHIT3 | Chr17: 31915085-31918085 | ZNHIT3, Chr17: 31915085-31918085 |
| SEPN1 | Chr1: 25997753-26000753 | SEPN1, Chr1: 25997753-26000753 |
| CPAMD8 | Chr19: 16997125-17000125 | CPAMD8, Chr19: 16997125-17000125 |
| ULK3 | Chr15: 72921105-72924105 | ULK3, Chr15: 72921105-72924105 |
| WDR23 | Chr14: 23652360-23655360 | WDR23, Chr14: 23652360-23655360 |
| STX1B | Chr16: 30927830-30930830 | STX1B, Chr16: 30927830-30930830 |
| PRICKLE3 | Chr23: 48928220-48931220 | PRICKLE3, Chr23: 48928220-48931220 |
| THOC6 | Chr16: 3012593-3015593 | THOC6, Chr16: 3012593-3015593 |
| GSR | Chr8: 30703485-30706485 | GSR, Chr8: 30703485-30706485 |
| MTX1 | Chr1: 153443613-153446613 | MTX1, Chr1: 153443613-153446613 |
| CCDC24 | Chr1: 44228366-44231366 | CCDC24, Chr1: 44228366-44231366 |
| PDCD10 | Chr3: 168933824-168936824 | PDCD10, Chr3: 168933824-168936824 |
| PDCD10 | Chr3: 168933845-168936845 | PDCD10, Chr3: 168933845-168936845 |
| TM6SF2 | Chr19: 19243574-19246574 | TM6SF2, Chr19: 19243574-19246574 |
| PDCD10 | Chr3: 168933788-168936788 | PDCD10, Chr3: 168933788-168936788 |
| NCAM2 | Chr21: 21291003-21294003 | NCAM2, Chr21: 21291003-21294003 |
| MCAT | Chr22: 41867847-41870847 | MCAT, Chr22: 41867847-41870847 |
| TMEM192 | Chr4: 166251974-166254974 | TMEM192, Chr4: 166251974-166254974 |
| NUAK2 | Chr1: 203556006-203559006 | NUAK2, Chr1: 203556006-203559006 |
| DDOST | Chr1: 20859124-20862124 | DDOST, Chr1: 20859124-20862124 |
| C8orf76 | Chr8: 124321298-124324298 | C8orf76, Chr8: 124321298-124324298 |
| RHBDF2 | Chr17: 72007603-72010603 | RHBDF2, Chr17: 72007603-72010603 |
| KRTCAP2 | Chr1: 153410928-153413928 | KRTCAP2, Chr1: 153410928-153413928 |
| IFI30 | Chr19: 18144078-18147078 | IFI30, Chr19: 18144078-18147078 |
| SCARB1 | Chr12: 123912972-123915972 | SCARB1, Chr12: 123912972-123915972 |
| ATXN2 | Chr12: 110520363-110523363 | ATXN2, Chr12: 110520363-110523363 |
| APITD1 | Chr1: 10411245-10414245 | APITD1, Chr1: 10411245-10414245 |
| TNFRSF10D | Chr8: 23075985-23078985 | TNFRSF10D, Chr8: 23075985-23078985 |
| MEIS3 | Chr19: 52613097-52616097 | MEIS3, Chr19: 52613097-52616097 |
| GJB3 | Chr1: 35017876-35020876 | GJB3, Chr1: 35017876-35020876 |
| HIST1H2BK | Chr6: 27221098-27224098 | HIST1H2BK, Chr6: 27221098-27224098 |
| TLR5 | Chr1: 221381747-221384747 | TLR5, Chr1: 221381747-221384747 |
| IL27RA | Chr19: 14001761-14004761 | IL27RA, Chr19: 14001761-14004761 |
| RILP | Chr17: 1498642-1501642 | RILP, Chr17: 1498642-1501642 |
| MRAS | Chr3: 139547814-139550814 | MRAS, Chr3: 139547814-139550814 |
| NPW | Chr16: 2008021-2011021 | NPW, Chr16: 2008021-2011021 |
| HOXB7 | Chr17: 44041882-44044882 | HOXB7, Chr17: 44041882-44044882 |
| TCF4 | Chr18: 51405358-51408358 | TCF4, Chr18: 51405358-51408358 |
| PLEKHG5 | Chr1: 6467101-6470101 | PLEKHG5, Chr1: 6467101-6470101 |
| AP1G2 | Chr14: 23105619-23108619 | AP1G2, Chr14: 23105619-23108619 |
| 6-Sep | Chr23: 118709861-118712861 | 40792, Chr23: 118709861-118712861 |
| RDHE2 | Chr8: 57394295-57397295 | RDHE2, Chr8: 57394295-57397295 |
| TSHR | Chr14: 80490121-80493121 | TSHR, Chr14: 80490121-80493121 |
| CA10 | Chr17: 47590660-47593660 | CA10, Chr17: 47590660-47593660 |
| CA10 | Chr17: 47590876-47593876 | CA10, Chr17: 47590876-47593876 |
| PADI4 | Chr1: 17505778-17508778 | PADI4, Chr1: 17505778-17508778 |
| PBX2 | Chr6: 32264441-32267441 | PBX2, Chr6: 32264441-32267441 |
| ACSS1 | Chr20: 24986116-24989116 | ACSS1, Chr20: 24986116-24989116 |
| VCAN | Chr5: 82801785-82804785 | VCAN, Chr5: 82801785-82804785 |
| C6orf223 | Chr6: 44074816-44077816 | C6orf223, Chr6: 44074816-44077816 |
| PCDHGA10 | Chr5: 140771426-140774426 | PCDHGA10, Chr5: 140771426-140774426 |
| PLEKHG4 | Chr16: 65867413-65870413 | PLEKHG4, Chr16: 65867413-65870413 |
| OSBPL3 | Chr7: 24984785-24987785 | OSBPL3, Chr7: 24984785-24987785 |
| EMD | Chr23: 153259480-153262480 | EMD, Chr23: 153259480-153262480 |
| DUSP6 | Chr12: 88268927-88271927 | DUSP6, Chr12: 88268927-88271927 |
| CAMKV | Chr3: 49880873-49883873 | CAMKV, Chr3: 49880873-49883873 |
| TP53I13 | Chr17: 24918364-24921364 | TP53I13, Chr17: 24918364-24921364 |
| EBF2 | Chr8: 25956809-25959809 | EBF2, Chr8: 25956809-25959809 |
| HCFC1R1 | Chr16: 3012788-3015788 | HCFC1R1, Chr16: 3012788-3015788 |
| CCDC64B | Chr16: 3024043-3027043 | CCDC64B, Chr16: 3024043-3027043 |
| CDK5 | Chr7: 150384429-150387429 | CDK5, Chr7: 150384429-150387429 |
| LIPG | Chr18: 45340924-45343924 | LIPG, Chr18: 45340924-45343924 |
| GALE | Chr1: 23997147-24000147 | GALE, Chr1: 23997147-24000147 |
| SPSB2 | Chr12: 6851213-6854213 | SPSB2, Chr12: 6851213-6854213 |
| RASD2 | Chr22: 34265797-34268797 | RASD2, Chr22: 34265797-34268797 |
| KLC4 | Chr6: 43134659-43137659 | KLC4, Chr6: 43134659-43137659 |
| INSRR | Chr1: 155093790-155096790 | INSRR, Chr1: 155093790-155096790 |
| HPD | Chr12: 120779652-120782652 | HPD, Chr12: 120779652-120782652 |
| C19orf55 | Chr19: 40939383-40942383 | C19orf55, Chr19: 40939383-40942383 |
| DAND5 | Chr19: 12939931-12942931 | DAND5, Chr19: 12939931-12942931 |
| SNTG1 | Chr8: 50985649-50988649 | SNTG1, Chr8: 50985649-50988649 |
| FLJ45983 | Chr10: 8133953-8136953 | FLJ45983, Chr10: 8133953-8136953 |
| PRCP | Chr11: 82287705-82290705 | PRCP, Chr11: 82287705-82290705 |
| THBS3 | Chr1: 153442814-153445814 | THBS3, Chr1: 153442814-153445814 |
| SOX9 | Chr17: 67627255-67630255 | SOX9, Chr17: 67627255-67630255 |
| SERPINI1 | Chr3: 168934625-168937625 | SERPINI1, Chr3: 168934625-168937625 |
| SHOX2 | Chr3: 159305130-159308130 | SHOX2, Chr3: 159305130-159308130 |
| SERPINI1 | Chr3: 168934704-168937704 | SERPINI1, Chr3: 168934704-168937704 |
| NOTCH1 | Chr9: 138558559-138561559 | NOTCH1, Chr9: 138558559-138561559 |
| MAF1 | Chr8: 145229792-145232792 | MAF1, Chr8: 145229792-145232792 |
| WNT5A | Chr3: 55494871-55497871 | WNT5A, Chr3: 55494871-55497871 |
| STK32C | Chr10: 133969967-133972967 | STK32C, Chr10: 133969967-133972967 |
| CAPZB | Chr1: 19683079-19686079 | CAPZB, Chr1: 19683079-19686079 |
| BEST1 | Chr11: 61472907-61475907 | BEST1, Chr11: 61472907-61475907 |
| KRT13 | Chr17: 36913891-36916891 | KRT13, Chr17: 36913891-36916891 |
| RELN | Chr7: 103415698-103418698 | RELN, Chr7: 103415698-103418698 |
| CITED2 | Chr6: 139735978-139738978 | CITED2, Chr6: 139735978-139738978 |
| UNC13A | Chr19: 17658901-17661901 | UNC13A, Chr19: 17658901-17661901 |
| SMARCD3 | Chr7: 150575182-150578182 | SMARCD3, Chr7: 150575182-150578182 |
| LMTK3 | Chr19: 53706758-53709758 | LMTK3, Chr19: 53706758-53709758 |
| HIST1H2AH | Chr6: 27221386-27224386 | HIST1H2AH, Chr6: 27221386-27224386 |
| KLC4 | Chr6: 43134106-43137106 | KLC4, Chr6: 43134106-43137106 |
| CA2 | Chr8: 86561882-86564882 | CA2, Chr8: 86561882-86564882 |
| SQLE | Chr8: 126078400-126081400 | SQLE, Chr8: 126078400-126081400 |
| PSMD5 | Chr9: 122643527-122646527 | PSMD5, Chr9: 122643527-122646527 |
| RAET1G | Chr6: 150284407-150287407 | RAET1G, Chr6: 150284407-150287407 |
| ADRM1 | Chr20: 60309921-60312921 | ADRM1, Chr20: 60309921-60312921 |
| KIAA1737 | Chr14: 76632830-76635830 | KIAA1737, Chr14: 76632830-76635830 |
| IGSF8 | Chr1: 158333532-158336532 | IGSF8, Chr1: 158333532-158336532 |
| KCNB2 | Chr8: 73610679-73613679 | KCNB2, Chr8: 73610679-73613679 |
| RAET1L | Chr6: 150386861-150389861 | RAET1L, Chr6: 150386861-150389861 |
| TADA3L | Chr3: 9807920-9810920 | TADA3L, Chr3: 9807920-9810920 |
| VPS37D | Chr7: 72718609-72721609 | VPS37D, Chr7: 72718609-72721609 |
| SYNE1 | Chr6: 152998179-153001179 | SYNE1, Chr6: 152998179-153001179 |
| TRIM47 | Chr17: 71384751-71387751 | TRIM47, Chr17: 71384751-71387751 |
| BIN1 | Chr2: 127579834-127582834 | BIN1, Chr2: 127579834-127582834 |
| CSGlcA-T | Chr7: 150559017-150562017 | CSGlcA-T, Chr7: 150559017-150562017 |
| STEAP3 | Chr2: 119696353-119699353 | STEAP3, Chr2: 119696353-119699353 |
| ZSCAN18 | Chr19: 63299889-63302889 | ZSCAN18, Chr19: 63299889-63302889 |
| ZBTB9 | Chr6: 33528833-33531833 | ZBTB9, Chr6: 33528833-33531833 |
| PDE1B | Chr12: 53228170-53231170 | PDE1B, Chr12: 53228170-53231170 |
| ATP5SL | Chr19: 46636150-46639150 | ATP5SL, Chr19: 46636150-46639150 |
| SLC27A3 | Chr1: 152012891-152015891 | SLC27A3, Chr1: 152012891-152015891 |
| POLE4 | Chr2: 75037782-75040782 | POLE4, Chr2: 75037782-75040782 |
| ZNF839 | Chr14: 101854348-101857348 | ZNF839, Chr14: 101854348-101857348 |
| C7orf46 | Chr7: 23684773-23687773 | C7orf46, Chr7: 23684773-23687773 |
| DGKK | Chr23: 50228977-50231977 | DGKK, Chr23: 50228977-50231977 |
| CLCN2 | Chr3: 185560585-185563585 | CLCN2, Chr3: 185560585-185563585 |
| CHST13 | Chr3: 127724365-127727365 | CHST13, Chr3: 127724365-127727365 |
| C19orf15 | Chr19: 43516782-43519782 | C19orf15, Chr19: 43516782-43519782 |
| MSX2 | Chr5: 174082680-174085680 | MSX2, Chr5: 174082680-174085680 |
| LDLRAP1 | Chr1: 25741162-25744162 | LDLRAP1, Chr1: 25741162-25744162 |
| CARS | Chr11: 3033747-3036747 | CARS, Chr11: 3033747-3036747 |
| HECTD3 | Chr1: 45248114-45251114 | HECTD3, Chr1: 45248114-45251114 |
| SHMT2 | Chr12: 55908318-55911318 | SHMT2, Chr12: 55908318-55911318 |
| CST3 | Chr20: 23565074-23568074 | CST3, Chr20: 23565074-23568074 |
| MXRA7 | Chr17: 72217151-72220151 | MXRA7, Chr17: 72217151-72220151 |
| MFAP2 | Chr1: 17178226-17181226 | MFAP2, Chr1: 17178226-17181226 |
| ADCY6 | Chr12: 47467587-47470587 | ADCY6, Chr12: 47467587-47470587 |
| WDR46 | Chr6: 33363469-33366469 | WDR46, Chr6: 33363469-33366469 |
| TMEM9 | Chr1: 199388741-199391741 | TMEM9, Chr1: 199388741-199391741 |
| SNX31 | Chr8: 101729569-101732569 | SNX31, Chr8: 101729569-101732569 |
| SLC38A7 | Chr16: 57274675-57277675 | SLC38A7, Chr16: 57274675-57277675 |
| SYNE1 | Chr6: 152998727-153001727 | SYNE1, Chr6: 152998727-153001727 |
| TUBA1A | Chr12: 47867628-47870628 | TUBA1A, Chr12: 47867628-47870628 |
| IL17RD | Chr3: 57172943-57175943 | IL17RD, Chr3: 57172943-57175943 |
| PDE4B | Chr1: 66029280-66032280 | PDE4B, Chr1: 66029280-66032280 |
| RASAL1 | Chr12: 112056904-112059904 | RASAL1, Chr12: 112056904-112059904 |
| STMN2 | Chr8: 80684434-80687434 | STMN2, Chr8: 80684434-80687434 |
| TUB | Chr11: 8015255-8018255 | TUB, Chr11: 8015255-8018255 |
| EPHB3 | Chr3: 185760780-185763780 | EPHB3, Chr3: 185760780-185763780 |
| TNNI3 | Chr19: 60359412-60362412 | TNNI3, Chr19: 60359412-60362412 |
| OR2L13 | Chr1: 246165615-246168615 | OR2L13, Chr1: 246165615-246168615 |
| PTGS1 | Chr9: 124171549-124174549 | PTGS1, Chr9: 124171549-124174549 |
| ETV5 | Chr3: 187308095-187311095 | ETV5, Chr3: 187308095-187311095 |
| FAIM3 | Chr1: 205160466-205163466 | FAIM3, Chr1: 205160466-205163466 |
| KIAA1026 | Chr1: 15143501-15146501 | KIAA1026, Chr1: 15143501-15146501 |
| GNG8 | Chr19: 51828279-51831279 | GNG8, Chr19: 51828279-51831279 |
| CFH | Chr1: 194886130-194889130 | CFH, Chr1: 194886130-194889130 |
| LILRA6 | Chr19: 59437036-59440036 | LILRA6, Chr19: 59437036-59440036 |
| C2CD2 | Chr21: 42218368-42221368 | C2CD2, Chr21: 42218368-42221368 |
| S100A13 | Chr1: 151864868-151867868 | S100A13, Chr1: 151864868-151867868 |
| CMIP | Chr16: 80084954-80087954 | CMIP, Chr16: 80084954-80087954 |
| PPP3CA | Chr4: 102485876-102488876 | PPP3CA, Chr4: 102485876-102488876 |
| COL1A2 | Chr7: 93860308-93863308 | COL1A2, Chr7: 93860308-93863308 |
| C3orf70 | Chr3: 186351996-186354996 | C3orf70, Chr3: 186351996-186354996 |
| CST5 | Chr20: 23806880-23809880 | CST5, Chr20: 23806880-23809880 |
| IGFBP6 | Chr12: 51776202-51779202 | IGFBP6, Chr12: 51776202-51779202 |
| S100A13 | Chr1: 151865241-151868241 | S100A13, Chr1: 151865241-151868241 |
| CALCR | Chr7: 93040186-93043186 | CALCR, Chr7: 93040186-93043186 |
| C7orf20 | Chr7: 881216-884216 | C7orf20, Chr7: 881216-884216 |
| GALNT10 | Chr5: 153762029-153765029 | GALNT10, Chr5: 153762029-153765029 |
| LIPH | Chr3: 186751563-186754563 | LIPH, Chr3: 186751563-186754563 |
| KCNH7 | Chr2: 163401986-163404986 | KCNH7, Chr2: 163401986-163404986 |
| SLFN13 | Chr17: 30798385-30801385 | SLFN13, Chr17: 30798385-30801385 |
| PFDN1 | Chr5: 139661373-139664373 | PFDN1, Chr5: 139661373-139664373 |
| KCNQ3 | Chr8: 133560686-133563686 | KCNQ3, Chr8: 133560686-133563686 |
| FXYD3 | Chr19: 40297138-40300138 | FXYD3, Chr19: 40297138-40300138 |
| TTR | Chr18: 27424337-27427337 | TTR, Chr18: 27424337-27427337 |
| FAM5B | Chr1: 175405755-175408755 | FAM5B, Chr1: 175405755-175408755 |
| RAB22A | Chr20: 56316676-56319676 | RAB22A, Chr20: 56316676-56319676 |
| SLC28A1 | Chr15: 83227416-83230416 | SLC28A1, Chr15: 83227416-83230416 |
| GABRA1 | Chr5: 161206975-161209975 | GABRA1, Chr5: 161206975-161209975 |
| SIGLEC7 | Chr19: 56335869-56338869 | SIGLEC7, Chr19: 56335869-56338869 |
| CCR6 | Chr6: 167454730-167457730 | CCR6, Chr6: 167454730-167457730 |
| NXPH1 | Chr7: 8438609-8441609 | NXPH1, Chr7: 8438609-8441609 |
| MRPL46 | Chr15: 86810137-86813137 | MRPL46, Chr15: 86810137-86813137 |
| SNX32 | Chr11: 65356485-65359485 | SNX32, Chr11: 65356485-65359485 |
| RGS12 | Chr4: 3284171-3287171 | RGS12, Chr4: 3284171-3287171 |
| UNQ1940 | Chr7: 135082487-135085487 | UNQ1940, Chr7: 135082487-135085487 |
| PEX19 | Chr1: 158520055-158523055 | PEX19, Chr1: 158520055-158523055 |
| S100A13 | Chr1: 151865839-151868839 | S100A13, Chr1: 151865839-151868839 |
| CCL16 | Chr17: 31331136-31334136 | CCL16, Chr17: 31331136-31334136 |
| OR2B11 | Chr1: 245680407-245683407 | OR2B11, Chr1: 245680407-245683407 |
| DKFZP586H2123 | Chr11: 35502252-35505252 | DKFZP586H2123, Chr11: 35502252-35505252 |
| PTPRO | Chr12: 15365253-15368253 | PTPRO, Chr12: 15365253-15368253 |
| GABRA1 | Chr5: 161206619-161209619 | GABRA1, Chr5: 161206619-161209619 |
| MAPK10 | Chr4: 87591807-87594807 | MAPK10, Chr4: 87591807-87594807 |
| MRPS11 | Chr15: 86810187-86813187 | MRPS11, Chr15: 86810187-86813187 |
| SLC6A15 | Chr12: 83829205-83832205 | SLC6A15, Chr12: 83829205-83832205 |
| HRASLS | Chr3: 194440111-194443111 | HRASLS, Chr3: 194440111-194443111 |
| AP3M1 | Chr10: 75579332-75582332 | AP3M1, Chr10: 75579332-75582332 |
| NDRG1 | Chr8: 134377180-134380180 | NDRG1, Chr8: 134377180-134380180 |
| SAA2 | Chr11: 18225258-18228258 | SAA2, Chr11: 18225258-18228258 |
| ABHD1 | Chr2: 27198660-27201660 | ABHD1, Chr2: 27198660-27201660 |
| SLC5A1 | Chr22: 30767758-30770758 | SLC5A1, Chr22: 30767758-30770758 |
| BST2 | Chr19: 17375884-17378884 | BST2, Chr19: 17375884-17378884 |
| CCDC33 | Chr15: 72396452-72399452 | CCDC33, Chr15: 72396452-72399452 |
| WDR66 | Chr12: 120839362-120842362 | WDR66, Chr12: 120839362-120842362 |
| ESR1 | Chr6: 152167000-152170000 | ESR1, Chr6: 152167000-152170000 |
| CPEB1 | Chr15: 81036128-81039128 | CPEB1, Chr15: 81036128-81039128 |
| ADIPOQ | Chr3: 188041656-188044656 | ADIPOQ, Chr3: 188041656-188044656 |
| C13orf26 | Chr13: 30403333-30406333 | C13orf26, Chr13: 30403333-30406333 |
| HOMEZ | Chr14: 22823649-22826649 | HOMEZ, Chr14: 22823649-22826649 |
| GDPD2 | Chr23: 69558215-69561215 | GDPD2, Chr23: 69558215-69561215 |
| CAPN3 | Chr15: 40480379-40483379 | CAPN3, Chr15: 40480379-40483379 |
| FCRL6 | Chr1: 158037296-158040296 | FCRL6, Chr1: 158037296-158040296 |
| CIITA | Chr16: 10877055-10880055 | CIITA, Chr16: 10877055-10880055 |
| C1orf87 | Chr1: 60310514-60313514 | C1orf87, Chr1: 60310514-60313514 |
| KALRN | Chr3: 125294774-125297774 | KALRN, Chr3: 125294774-125297774 |
| DPY30 | Chr2: 32116848-32119848 | DPY30, Chr2: 32116848-32119848 |
| SLC17A8 | Chr12: 99273624-99276624 | SLC17A8, Chr12: 99273624-99276624 |
| IL6 | Chr7: 22731842-22734842 | IL6, Chr7: 22731842-22734842 |
| CALB1 | Chr8: 91162783-91165783 | CALB1, Chr8: 91162783-91165783 |
| SST | Chr3: 188869395-188872395 | SST, Chr3: 188869395-188872395 |
| SPG3A | Chr14: 50094992-50097992 | SPG3A, Chr14: 50094992-50097992 |
| ADK | Chr10: 75579470-75582470 | ADK, Chr10: 75579470-75582470 |
| SPARC | Chr5: 151045210-151048210 | SPARC, Chr5: 151045210-151048210 |
| FHL2 | Chr2: 105419892-105422892 | FHL2, Chr2: 105419892-105422892 |
| MOG | Chr6: 29731287-29734287 | MOG, Chr6: 29731287-29734287 |
| HAS1 | Chr19: 56917533-56920533 | HAS1, Chr19: 56917533-56920533 |
| C4orf8 | Chr4: 2595456-2598456 | C4orf8, Chr4: 2595456-2598456 |
| CCDC49 | Chr17: 34233615-34236615 | CCDC49, Chr17: 34233615-34236615 |
| GTSF1 | Chr12: 53152114-53155114 | GTSF1, Chr12: 53152114-53155114 |
| BTBD16 | Chr10: 124019310-124022310 | BTBD16, Chr10: 124019310-124022310 |
| PCBP3 | Chr21: 46092802-46095802 | PCBP3, Chr21: 46092802-46095802 |
| RLBP1L1 | Chr8: 62361603-62364603 | RLBP1L1, Chr8: 62361603-62364603 |
| THBS2 | Chr6: 169394562-169397562 | THBS2, Chr6: 169394562-169397562 |
| ZFAND2A | Chr7: 1164824-1167824 | ZFAND2A, Chr7: 1164824-1167824 |
| ZNF572 | Chr8: 126053232-126056232 | ZNF572, Chr8: 126053232-126056232 |
| ZNF483 | Chr9: 113325767-113328767 | ZNF483, Chr9: 113325767-113328767 |
| UGT3A1 | Chr5: 36025756-36028756 | UGT3A1, Chr5: 36025756-36028756 |
| NCOA4 | Chr10: 51240872-51243872 | NCOA4, Chr10: 51240872-51243872 |
| UBR2 | Chr6: 42638535-42641535 | UBR2, Chr6: 42638535-42641535 |
| ZNF285A | Chr19: 49596117-49599117 | ZNF285A, Chr19: 49596117-49599117 |
| CLCN1 | Chr7: 142721840-142724840 | CLCN1, Chr7: 142721840-142724840 |
| BIRC3 | Chr11: 101691903-101694903 | BIRC3, Chr11: 101691903-101694903 |
| MMP7 | Chr11: 101905188-101908188 | MMP7, Chr11: 101905188-101908188 |
| LMOD1 | Chr1: 200180839-200183839 | LMOD1, Chr1: 200180839-200183839 |
| ZCCHC13 | Chr23: 73439249-73442249 | ZCCHC13, Chr23: 73439249-73442249 |
| LDLRAD1 | Chr1: 54254891-54257891 | LDLRAD1, Chr1: 54254891-54257891 |
| CA3 | Chr8: 86536209-86539209 | CA3, Chr8: 86536209-86539209 |
| TSPYL6 | Chr2: 54335413-54338413 | TSPYL6, Chr2: 54335413-54338413 |
| WNT5B | Chr12: 1607172-1610172 | WNT5B, Chr12: 1607172-1610172 |
| CHRNB1 | Chr17: 7287629-7290629 | CHRNB1, Chr17: 7287629-7290629 |
| SV2A | Chr1: 148154554-148157554 | SV2A, Chr1: 148154554-148157554 |
| FAM151A | Chr1: 54860288-54863288 | FAM151A, Chr1: 54860288-54863288 |
| ANKRD45 | Chr1: 171904124-171907124 | ANKRD45, Chr1: 171904124-171907124 |
| NRXN1 | Chr2: 51111678-51114678 | NRXN1, Chr2: 51111678-51114678 |
| PCNP | Chr3: 102774231-102777231 | PCNP, Chr3: 102774231-102777231 |
| GABRA1 | Chr5: 161206017-161209017 | GABRA1, Chr5: 161206017-161209017 |
| TAAR5 | Chr6: 132951018-132954018 | TAAR5, Chr6: 132951018-132954018 |
| C14orf152 | Chr14: 93453510-93456510 | C14orf152, Chr14: 93453510-93456510 |
| KCNS1 | Chr20: 43161667-43164667 | KCNS1, Chr20: 43161667-43164667 |
| OR10P1 | Chr12: 54315442-54318442 | OR10P1, Chr12: 54315442-54318442 |
| JAKMIP2 | Chr5: 147140945-147143945 | JAKMIP2, Chr5: 147140945-147143945 |
| STAR | Chr8: 38126257-38129257 | STAR, Chr8: 38126257-38129257 |
| CD244 | Chr1: 159097769-159100769 | CD244, Chr1: 159097769-159100769 |
| KRTAP6-3 | Chr21: 30885129-30888129 | KRTAP6-3, Chr21: 30885129-30888129 |
| SPAG17 | Chr1: 118527857-118530857 | SPAG17, Chr1: 118527857-118530857 |
| C1orf114 | Chr1: 167661794-167664794 | C1orf114, Chr1: 167661794-167664794 |
| LRRC3B | Chr3: 26637803-26640803 | LRRC3B, Chr3: 26637803-26640803 |
| KCNC2 | Chr12: 73888278-73891278 | KCNC2, Chr12: 73888278-73891278 |
| C8ORFK36 | Chr8: 124732871-124735871 | C8ORFK36, Chr8: 124732871-124735871 |
| EYA1 | Chr8: 72435521-72438521 | EYA1, Chr8: 72435521-72438521 |
| FUT9 | Chr6: 96569065-96572065 | FUT9, Chr6: 96569065-96572065 |
| ZNF394 | Chr7: 98934313-98937313 | ZNF394, Chr7: 98934313-98937313 |
| AIFM2 | Chr10: 71561196-71564196 | AIFM2, Chr10: 71561196-71564196 |
| PIPOX | Chr17: 24392543-24395543 | PIPOX, Chr17: 24392543-24395543 |
| SLC22A13 | Chr3: 38280806-38283806 | SLC22A13, Chr3: 38280806-38283806 |
| TTLL6 | Chr17: 44225185-44228185 | TTLL6, Chr17: 44225185-44228185 |
| PRSS16 | Chr6: 27321986-27324986 | PRSS16, Chr6: 27321986-27324986 |
| RASSF4 | Chr10: 44773724-44776724 | RASSF4, Chr10: 44773724-44776724 |
| CAMP | Chr3: 48238365-48241365 | CAMP, Chr3: 48238365-48241365 |
| ARMC3 | Chr10: 23255459-23258459 | ARMC3, Chr10: 23255459-23258459 |
| POPDC2 | Chr3: 120860594-120863594 | POPDC2, Chr3: 120860594-120863594 |
| FAM174A | Chr5: 99897522-99900522 | FAM174A, Chr5: 99897522-99900522 |
| TACR3 | Chr4: 104858922-104861922 | TACR3, Chr4: 104858922-104861922 |
| SLC2A9 | Chr4: 9649470-9652470 | SLC2A9, Chr4: 9649470-9652470 |
| SLC22A4 | Chr5: 131656543-131659543 | SLC22A4, Chr5: 131656543-131659543 |
| S100A1 | Chr1: 151865996-151868996 | S100A1, Chr1: 151865996-151868996 |
| OR2G6 | Chr1: 246750070-246753070 | OR2G6, Chr1: 246750070-246753070 |
| LHCGR | Chr2: 48834884-48837884 | LHCGR, Chr2: 48834884-48837884 |
| ZBBX | Chr3: 168579265-168582265 | ZBBX, Chr3: 168579265-168582265 |
| ARHGEF19 | Chr1: 16410191-16413191 | ARHGEF19, Chr1: 16410191-16413191 |
| KCNA4 | Chr11: 29993564-29996564 | KCNA4, Chr11: 29993564-29996564 |
| CAPN9 | Chr1: 228948252-228951252 | CAPN9, Chr1: 228948252-228951252 |
| RGS16 | Chr1: 180838671-180841671 | RGS16, Chr1: 180838671-180841671 |
| GABRA1 | Chr5: 161205762-161208762 | GABRA1, Chr5: 161205762-161208762 |
| COL22A1 | Chr8: 139993918-139996918 | COL22A1, Chr8: 139993918-139996918 |
| GALT | Chr9: 34635134-34638134 | GALT, Chr9: 34635134-34638134 |
| CAPN9 | Chr1: 228948283-228951283 | CAPN9, Chr1: 228948283-228951283 |
| CCR2 | Chr3: 46368863-46371863 | CCR2, Chr3: 46368863-46371863 |
| LAIR1 | Chr19: 59567033-59570033 | LAIR1, Chr19: 59567033-59570033 |
| MAGEC2 | Chr23: 141119242-141122242 | MAGEC2, Chr23: 141119242-141122242 |
| TMEM41A | Chr3: 186697996-186700996 | TMEM41A, Chr3: 186697996-186700996 |
| CYYR1 | Chr21: 26865952-26868952 | CYYR1, Chr21: 26865952-26868952 |
| VTCN1 | Chr1: 117553572-117556572 | VTCN1, Chr1: 117553572-117556572 |
| KRT35 | Chr17: 36889418-36892418 | KRT35, Chr17: 36889418-36892418 |
| TUBB4 | Chr19: 6451830-6454830 | TUBB4, Chr19: 6451830-6454830 |
| LRP1 | Chr12: 55807048-55810048 | LRP1, Chr12: 55807048-55810048 |
| GRM3 | Chr7: 86109665-86112665 | GRM3, Chr7: 86109665-86112665 |
| BACE1 | Chr11: 116690682-116693682 | BACE1, Chr11: 116690682-116693682 |
| DAB2IP | Chr9: 123543384-123546384 | DAB2IP, Chr9: 123543384-123546384 |
| CT45-1 | Chr23: 134673350-134676350 | CT45-1, Chr23: 134673350-134676350 |
| OR6S1 | Chr14: 20178190-20181190 | OR6S1, Chr14: 20178190-20181190 |
| SLIT3 | Chr5: 168659211-168662211 | SLIT3, Chr5: 168659211-168662211 |
| FLJ78302 | Chr3: 46368738-46371738 | FLJ78302, Chr3: 46368738-46371738 |
| GRM1 | Chr6: 146388974-146391974 | GRM1, Chr6: 146388974-146391974 |
| FAM75B | Chr9: 83865324-83868324 | FAM75B, Chr9: 83865324-83868324 |
| MYOM3 | Chr1: 24309752-24312752 | MYOM3, Chr1: 24309752-24312752 |
| LRRIQ4 | Chr3: 171020903-171023903 | LRRIQ4, Chr3: 171020903-171023903 |
| SIGLEC10 | Chr19: 56611763-56614763 | SIGLEC10, Chr19: 56611763-56614763 |
| DHH | Chr12: 47773369-47776369 | DHH, Chr12: 47773369-47776369 |
| HNRNPF | Chr10: 43210785-43213785 | HNRNPF, Chr10: 43210785-43213785 |
| RD3 | Chr1: 209731382-209734382 | RD3, Chr1: 209731382-209734382 |
| HTR3C | Chr3: 185252028-185255028 | HTR3C, Chr3: 185252028-185255028 |
| MYO7B | Chr2: 128008347-128011347 | MYO7B, Chr2: 128008347-128011347 |
| DENND2A | Chr7: 139947311-139950311 | DENND2A, Chr7: 139947311-139950311 |
| KLK4 | Chr19: 56104306-56107306 | KLK4, Chr19: 56104306-56107306 |
| SEMA4A | Chr1: 154388511-154391511 | SEMA4A, Chr1: 154388511-154391511 |
| S100A7A | Chr1: 151654123-151657123 | S100A7A, Chr1: 151654123-151657123 |
| GABRA1 | Chr5: 161205274-161208274 | GABRA1, Chr5: 161205274-161208274 |
| SIGLEC5 | Chr19: 56824030-56827030 | SIGLEC5, Chr19: 56824030-56827030 |
| ZNF608 | Chr5: 124107204-124110204 | ZNF608, Chr5: 124107204-124110204 |
| FAM43A | Chr3: 195886410-195889410 | FAM43A, Chr3: 195886410-195889410 |
| PCTK3 | Chr1: 203738849-203741849 | PCTK3, Chr1: 203738849-203741849 |
| FMOD | Chr1: 201585412-201588412 | FMOD, Chr1: 201585412-201588412 |
| ZNF385B | Chr2: 180432977-180435977 | ZNF385B, Chr2: 180432977-180435977 |
| PVALB | Chr22: 35543963-35546963 | PVALB, Chr22: 35543963-35546963 |
| VCX2 | Chr23: 8097808-8100808 | VCX2, Chr23: 8097808-8100808 |
| S100A5 | Chr1: 151779365-151782365 | S100A5, Chr1: 151779365-151782365 |
| OTX2 | Chr14: 56340598-56343598 | OTX2, Chr14: 56340598-56343598 |
| FLJ34931 | Chr2: 29149131-29152131 | FLJ34931, Chr2: 29149131-29152131 |
| L1CAM | Chr23: 152793005-152796005 | L1CAM, Chr23: 152793005-152796005 |
| KISS1 | Chr1: 202430742-202433742 | KISS1, Chr1: 202430742-202433742 |
| BRSK1 | Chr19: 60485845-60488845 | BRSK1, Chr19: 60485845-60488845 |
| HOXC6 | Chr12: 52706960-52709960 | HOXC6, Chr12: 52706960-52709960 |
| TRIM9 | Chr14: 50630672-50633672 | TRIM9, Chr14: 50630672-50633672 |
| HNRNPF | Chr10: 43211300-43214300 | HNRNPF, Chr10: 43211300-43214300 |
| IGF2 | Chr11: 2125909-2128909 | IGF2, Chr11: 2125909-2128909 |
| MYT1 | Chr20: 62264770-62267770 | MYT1, Chr20: 62264770-62267770 |
| CSF3R | Chr1: 36719596-36722596 | CSF3R, Chr1: 36719596-36722596 |
| FCN3 | Chr1: 27572402-27575402 | FCN3, Chr1: 27572402-27575402 |
| HES1 | Chr3: 195335127-195338127 | HES1, Chr3: 195335127-195338127 |
| STRA8 | Chr7: 134565770-134568770 | STRA8, Chr7: 134565770-134568770 |
| TIAF1 | Chr17: 24425253-24428253 | TIAF1, Chr17: 24425253-24428253 |
| DEFB132 | Chr20: 184876-187876 | DEFB132, Chr20: 184876-187876 |
| ORM1 | Chr9: 116123623-116126623 | ORM1, Chr9: 116123623-116126623 |
| ZBP1 | Chr20: 55627435-55630435 | ZBP1, Chr20: 55627435-55630435 |
| RETNLB | Chr3: 109957320-109960320 | RETNLB, Chr3: 109957320-109960320 |
| RTP1 | Chr3: 188396467-188399467 | RTP1, Chr3: 188396467-188399467 |
| TNRC6C | Chr17: 73555088-73558088 | TNRC6C, Chr17: 73555088-73558088 |
| TSC22D4 | Chr7: 99913338-99916338 | TSC22D4, Chr7: 99913338-99916338 |
| TESSP5 | Chr3: 46759749-46762749 | TESSP5, Chr3: 46759749-46762749 |
| HOXB13 | Chr17: 44159610-44162610 | HOXB13, Chr17: 44159610-44162610 |
| RBM23 | Chr14: 22456736-22459736 | RBM23, Chr14: 22456736-22459736 |
| PRDM14 | Chr8: 71144616-71147616 | PRDM14, Chr8: 71144616-71147616 |
| HMX3 | Chr10: 124884056-124887056 | HMX3, Chr10: 124884056-124887056 |
| SOX11 | Chr2: 5748749-5751749 | SOX11, Chr2: 5748749-5751749 |
| POU3F3 | Chr2: 104836900-104839900 | POU3F3, Chr2: 104836900-104839900 |
| MYOD1 | Chr11: 17696185-17699185 | MYOD1, Chr11: 17696185-17699185 |
| INSM1 | Chr20: 20295264-20298264 | INSM1, Chr20: 20295264-20298264 |
| FZD8 | Chr10: 35968868-35971868 | FZD8, Chr10: 35968868-35971868 |
| EMX1 | Chr2: 72996611-72999611 | EMX1, Chr2: 72996611-72999611 |
| MAZ | Chr16: 29723855-29726855 | MAZ, Chr16: 29723855-29726855 |
| CAMK2N2 | Chr3: 185460445-185463445 | CAMK2N2, Chr3: 185460445-185463445 |
| MAFB | Chr20: 38749790-38752790 | MAFB, Chr20: 38749790-38752790 |
| UNCX | Chr7: 1237679-1240679 | UNCX, Chr7: 1237679-1240679 |
| MEX3B | Chr15: 80123916-80126916 | MEX3B, Chr15: 80123916-80126916 |
| CTBP2 | Chr10: 126837593-126840593 | CTBP2, Chr10: 126837593-126840593 |
| GFI1 | Chr1: 92720444-92723444 | GFI1, Chr1: 92720444-92723444 |
| LOC116236 | Chr17: 24916674-24919674 | LOC116236, Chr17: 24916674-24919674 |
| NKX2-1 | Chr14: 36057154-36060154 | NKX2-1, Chr14: 36057154-36060154 |
| FLOT1 | Chr6: 30816932-30819932 | FLOT1, Chr6: 30816932-30819932 |
| GNAS | Chr20: 56898319-56901319 | GNAS, Chr20: 56898319-56901319 |
| GNAS | Chr20: 56898320-56901320 | GNAS, Chr20: 56898320-56901320 |
| CEBPA | Chr19: 38483660-38486660 | CEBPA, Chr19: 38483660-38486660 |
| WBSCR17 | Chr7: 70234224-70237224 | WBSCR17, Chr7: 70234224-70237224 |
| IGF2BP1 | Chr17: 44428272-44431272 | IGF2BP1, Chr17: 44428272-44431272 |
| KCNA3 | Chr1: 111017678-111020678 | KCNA3, Chr1: 111017678-111020678 |
| ABCA3 | Chr16: 2329248-2332248 | ABCA3, Chr16: 2329248-2332248 |
| RUNX3 | Chr1: 25127857-25130857 | RUNX3, Chr1: 25127857-25130857 |
| NFATC1 | Chr18: 75255259-75258259 | NFATC1, Chr18: 75255259-75258259 |
| RAX | Chr18: 55090105-55093105 | RAX, Chr18: 55090105-55093105 |
| GDF7 | Chr2: 20728404-20731404 | GDF7, Chr2: 20728404-20731404 |
| C16orf72 | Chr16: 9091537-9094537 | C16orf72, Chr16: 9091537-9094537 |
| C16orf5 | Chr16: 4527317-4530317 | C16orf5, Chr16: 4527317-4530317 |
| TRIM67 | Chr1: 229363796-229366796 | TRIM67, Chr1: 229363796-229366796 |
| RASGRP2 | Chr11: 64267405-64270405 | RASGRP2, Chr11: 64267405-64270405 |
| NR2E1 | Chr6: 108592454-108595454 | NR2E1, Chr6: 108592454-108595454 |
| GNAS | Chr20: 56898663-56901663 | GNAS, Chr20: 56898663-56901663 |
| NEFL | Chr8: 24868543-24871543 | NEFL, Chr8: 24868543-24871543 |
| ZNF141 | Chr4: 320095-323095 | ZNF141, Chr4: 320095-323095 |
| SKI | Chr1: 2148493-2151493 | SKI, Chr1: 2148493-2151493 |
| RPP25 | Chr15: 73035328-73038328 | RPP25, Chr15: 73035328-73038328 |
| EPHA4 | Chr2: 222143754-222146754 | EPHA4, Chr2: 222143754-222146754 |
| STX16 | Chr20: 56658233-56661233 | STX16, Chr20: 56658233-56661233 |
| EBF3 | Chr10: 131650581-131653581 | EBF3, Chr10: 131650581-131653581 |
| RPS6KA3 | Chr23: 20193171-20196171 | RPS6KA3, Chr23: 20193171-20196171 |
| BCL2 | Chr18: 59136093-59139093 | BCL2, Chr18: 59136093-59139093 |
| WT1 | Chr11: 32412163-32415163 | WT1, Chr11: 32412163-32415163 |
| C9orf4 | Chr9: 110967892-110970892 | C9orf4, Chr9: 110967892-110970892 |
| SIRPA | Chr20: 1822325-1825325 | SIRPA, Chr20: 1822325-1825325 |
| G3BP1 | Chr5: 151130168-151133168 | G3BP1, Chr5: 151130168-151133168 |
| MEIS2 | Chr15: 35178496-35181496 | MEIS2, Chr15: 35178496-35181496 |
| PRDM12 | Chr9: 132528301-132531301 | PRDM12, Chr9: 132528301-132531301 |
| IRX1 | Chr5: 3647667-3650667 | IRX1, Chr5: 3647667-3650667 |
| CYP26B1 | Chr2: 72226971-72229971 | CYP26B1, Chr2: 72226971-72229971 |
| GHSR | Chr3: 173647397-173650397 | GHSR, Chr3: 173647397-173650397 |
| SAMD5 | Chr6: 147870255-147873255 | SAMD5, Chr6: 147870255-147873255 |
| PAX5 | Chr9: 37022976-37025976 | PAX5, Chr9: 37022976-37025976 |
| PCID2 | Chr13: 112909484-112912484 | PCID2, Chr13: 112909484-112912484 |
| ADM | Chr11: 10281717-10284717 | ADM, Chr11: 10281717-10284717 |
| HS3ST3B1 | Chr17: 14143730-14146730 | HS3ST3B1, Chr17: 14143730-14146730 |
| OLIG3 | Chr6: 137855724-137858724 | OLIG3, Chr6: 137855724-137858724 |
| TSHZ3 | Chr19: 36530530-36533530 | TSHZ3, Chr19: 36530530-36533530 |
| NPR3 | Chr5: 32745921-32748921 | NPR3, Chr5: 32745921-32748921 |
| MFAP3L | Chr4: 171182504-171185504 | MFAP3L, Chr4: 171182504-171185504 |
| KCNC4 | Chr1: 110554087-110557087 | KCNC4, Chr1: 110554087-110557087 |
| TIMP2 | Chr17: 74431567-74434567 | TIMP2, Chr17: 74431567-74434567 |
| HMGA1 | Chr6: 34311127-34314127 | HMGA1, Chr6: 34311127-34314127 |
| IRX5 | Chr16: 53521111-53524111 | IRX5, Chr16: 53521111-53524111 |
| PENK | Chr8: 57519643-57522643 | PENK, Chr8: 57519643-57522643 |
| CDX2 | Chr13: 27439817-27442817 | CDX2, Chr13: 27439817-27442817 |
| MAEA | Chr4: 1272171-1275171 | MAEA, Chr4: 1272171-1275171 |
| IER3 | Chr6: 30818806-30821806 | IER3, Chr6: 30818806-30821806 |
| SEMA6A | Chr5: 115936950-115939950 | SEMA6A, Chr5: 115936950-115939950 |
| CUL4A | Chr13: 112910431-112913431 | CUL4A, Chr13: 112910431-112913431 |
| SALL1 | Chr16: 49741184-49744184 | SALL1, Chr16: 49741184-49744184 |
| PRKCE | Chr2: 45731046-45734046 | PRKCE, Chr2: 45731046-45734046 |
| TTYH2 | Chr17: 69719790-69722790 | TTYH2, Chr17: 69719790-69722790 |
| KIAA1543 | Chr19: 7565287-7568287 | KIAA1543, Chr19: 7565287-7568287 |
| HMGA1 | Chr6: 34311406-34314406 | HMGA1, Chr6: 34311406-34314406 |
| SHE | Chr1: 152739713-152742713 | SHE, Chr1: 152739713-152742713 |
| SFMBT1 | Chr3: 53052821-53055821 | SFMBT1, Chr3: 53052821-53055821 |
| MADCAM1 | Chr19: 445989-448989 | MADCAM1, Chr19: 445989-448989 |
| FEZF1 | Chr7: 121730335-121733335 | FEZF1, Chr7: 121730335-121733335 |
| SLIT2 | Chr4: 19862832-19865832 | SLIT2, Chr4: 19862832-19865832 |
| CPT1C | Chr19: 54884718-54887718 | CPT1C, Chr19: 54884718-54887718 |
| PER3 | Chr1: 7765849-7768849 | PER3, Chr1: 7765849-7768849 |
| SOX14 | Chr3: 138964768-138967768 | SOX14, Chr3: 138964768-138967768 |
| LRP2 | Chr2: 169925868-169928868 | LRP2, Chr2: 169925868-169928868 |
| LOC653319 | Chr16: 65773884-65776884 | LOC653319, Chr16: 65773884-65776884 |
| NRN1 | Chr6: 5951132-5954132 | NRN1, Chr6: 5951132-5954132 |
| C9orf85 | Chr9: 73714742-73717742 | C9orf85, Chr9: 73714742-73717742 |
| MCOLN2 | Chr1: 85233884-85236884 | MCOLN2, Chr1: 85233884-85236884 |
| SALL1 | Chr16: 49740509-49743509 | SALL1, Chr16: 49740509-49743509 |
| MEIS2 | Chr15: 35177389-35180389 | MEIS2, Chr15: 35177389-35180389 |
| EVX2 | Chr2: 176655436-176658436 | EVX2, Chr2: 176655436-176658436 |
| CACNA1E | Chr1: 179717838-179720838 | CACNA1E, Chr1: 179717838-179720838 |
| HLA-G | Chr6: 29901234-29904234 | HLA-G, Chr6: 29901234-29904234 |
| OTP | Chr5: 76968778-76971778 | OTP, Chr5: 76968778-76971778 |
| LUC7L | Chr16: 217950-220950 | LUC7L, Chr16: 217950-220950 |
| BVES | Chr6: 105689414-105692414 | BVES, Chr6: 105689414-105692414 |
| PACSIN1 | Chr6: 34540382-34543382 | PACSIN1, Chr6: 34540382-34543382 |
| CDH3 | Chr16: 67234151-67237151 | CDH3, Chr16: 67234151-67237151 |
| IKZF1 | Chr7: 50313423-50316423 | IKZF1, Chr7: 50313423-50316423 |
| E2F8 | Chr11: 19217583-19220583 | E2F8, Chr11: 19217583-19220583 |
| SHC3 | Chr9: 90982002-90985002 | SHC3, Chr9: 90982002-90985002 |
| BDNF | Chr11: 27698372-27701372 | BDNF, Chr11: 27698372-27701372 |
| LBXCOR1 | Chr15: 65903494-65906494 | LBXCOR1, Chr15: 65903494-65906494 |
| LRFN5 | Chr14: 41145593-41148593 | LRFN5, Chr14: 41145593-41148593 |
| VSX1 | Chr20: 25009267-25012267 | VSX1, Chr20: 25009267-25012267 |
| CCDC140 | Chr2: 222869609-222872609 | CCDC140, Chr2: 222869609-222872609 |
| MSX1 | Chr4: 4910792-4913792 | MSX1, Chr4: 4910792-4913792 |
| CYP26A1 | Chr10: 94822136-94825136 | CYP26A1, Chr10: 94822136-94825136 |
| CYP26A1 | Chr10: 94821721-94824721 | CYP26A1, Chr10: 94821721-94824721 |
| RNF152 | Chr18: 57709784-57712784 | RNF152, Chr18: 57709784-57712784 |
| EVX1 | Chr7: 27247188-27250188 | EVX1, Chr7: 27247188-27250188 |
| GPX7 | Chr1: 52839131-52842131 | GPX7, Chr1: 52839131-52842131 |
| TMEM26 | Chr10: 62881714-62884714 | TMEM26, Chr10: 62881714-62884714 |
| RAB34 | Chr17: 24067513-24070513 | RAB34, Chr17: 24067513-24070513 |
| FANCG | Chr9: 35068513-35071513 | FANCG, Chr9: 35068513-35071513 |
| C9orf19 | Chr9: 36125241-36128241 | C9orf19, Chr9: 36125241-36128241 |
| TEAD2 | Chr19: 54556026-54559026 | TEAD2, Chr19: 54556026-54559026 |
| ADAM12 | Chr10: 128065555-128068555 | ADAM12, Chr10: 128065555-128068555 |
| PPARG | Chr3: 12303935-12306935 | PPARG, Chr3: 12303935-12306935 |
| MRPL36 | Chr5: 1851456-1854456 | MRPL36, Chr5: 1851456-1854456 |
| CORO6 | Chr17: 24971120-24974120 | CORO6, Chr17: 24971120-24974120 |
| NDUFS6 | Chr5: 1853008-1856008 | NDUFS6, Chr5: 1853008-1856008 |
| RFX4 | Chr12: 105499662-105502662 | RFX4, Chr12: 105499662-105502662 |
| PIP4K2C | Chr12: 56269823-56272823 | PIP4K2C, Chr12: 56269823-56272823 |
| CALCA | Chr11: 14948908-14951908 | CALCA, Chr11: 14948908-14951908 |
| PCDHGA11 | Chr5: 140779220-140782220 | PCDHGA11, Chr5: 140779220-140782220 |
| TBR1 | Chr2: 161979365-161982365 | TBR1, Chr2: 161979365-161982365 |
| PCDH10 | Chr4: 134288419-134291419 | PCDH10, Chr4: 134288419-134291419 |
| RALYL | Chr8: 85258154-85261154 | RALYL, Chr8: 85258154-85261154 |
| TBX5 | Chr12: 113324586-113327586 | TBX5, Chr12: 113324586-113327586 |
| ARID3C | Chr9: 34616511-34619511 | ARID3C, Chr9: 34616511-34619511 |
| HIST1H2BI | Chr6: 26379682-26382682 | HIST1H2BI, Chr6: 26379682-26382682 |
| PPP1R13L | Chr19: 50598629-50601629 | PPP1R13L, Chr19: 50598629-50601629 |
| BARHL1 | Chr9: 134446313-134449313 | BARHL1, Chr9: 134446313-134449313 |
| HIST1H3G | Chr6: 26378091-26381091 | HIST1H3G, Chr6: 26378091-26381091 |
| TAL1 | Chr1: 47466530-47469530 | TAL1, Chr1: 47466530-47469530 |
| KCNIP2 | Chr10: 103588101-103591101 | KCNIP2, Chr10: 103588101-103591101 |
| CXCR7 | Chr2: 237141618-237144618 | CXCR7, Chr2: 237141618-237144618 |
| HOXB6 | Chr17: 44035833-44038833 | HOXB6, Chr17: 44035833-44038833 |
| PDPN | Chr1: 13783053-13786053 | PDPN, Chr1: 13783053-13786053 |
| BMP4 | Chr14: 53489520-53492520 | BMP4, Chr14: 53489520-53492520 |
| GBX2 | Chr2: 236739891-236742891 | GBX2, Chr2: 236739891-236742891 |
| FOXB2 | Chr9: 78822890-78825890 | FOXB2, Chr9: 78822890-78825890 |
| LMX1A | Chr1: 163590141-163593141 | LMX1A, Chr1: 163590141-163593141 |
| SLC32A1 | Chr20: 36785018-36788018 | SLC32A1, Chr20: 36785018-36788018 |
| TNFRSF10A | Chr8: 23137084-23140084 | TNFRSF10A, Chr8: 23137084-23140084 |
| GRHL3 | Chr1: 24520616-24523616 | GRHL3, Chr1: 24520616-24523616 |
| KCNC1 | Chr11: 17712570-17715570 | KCNC1, Chr11: 17712570-17715570 |
| GCHFR | Chr15: 38842076-38845076 | GCHFR, Chr15: 38842076-38845076 |
| GALNT10 | Chr5: 153548987-153551987 | GALNT10, Chr5: 153548987-153551987 |
| DNAJC14 | Chr12: 54508187-54511187 | DNAJC14, Chr12: 54508187-54511187 |
| PHF11 | Chr13: 48967028-48970028 | PHF11, Chr13: 48967028-48970028 |
| SLC25A20 | Chr3: 48909906-48912906 | SLC25A20, Chr3: 48909906-48912906 |
| ACVR2A | Chr2: 148317539-148320539 | ACVR2A, Chr2: 148317539-148320539 |
| CHST6 | Chr16: 74084927-74087927 | CHST6, Chr16: 74084927-74087927 |
| INTS9 | Chr8: 28801898-28804898 | INTS9, Chr8: 28801898-28804898 |
| CACNA1F | Chr23: 48975277-48978277 | CACNA1F, Chr23: 48975277-48978277 |
| TLX3 | Chr5: 170667392-170670392 | TLX3, Chr5: 170667392-170670392 |
| GFRA1 | Chr10: 118021466-118024466 | GFRA1, Chr10: 118021466-118024466 |
| CYP1B1 | Chr2: 38155327-38158327 | CYP1B1, Chr2: 38155327-38158327 |
| ZFP36L2 | Chr2: 43305749-43308749 | ZFP36L2, Chr2: 43305749-43308749 |
| ZNRF1 | Chr16: 73588915-73591915 | ZNRF1, Chr16: 73588915-73591915 |
| GATA6 | Chr18: 18001913-18004913 | GATA6, Chr18: 18001913-18004913 |
| NKX2-1 | Chr14: 36057667-36060667 | NKX2-1, Chr14: 36057667-36060667 |
| CACNA1D | Chr3: 53502570-53505570 | CACNA1D, Chr3: 53502570-53505570 |
| NR2F6 | Chr19: 17215651-17218651 | NR2F6, Chr19: 17215651-17218651 |
| KCNJ3 | Chr2: 155261838-155264838 | KCNJ3, Chr2: 155261838-155264838 |
| CYBRD1 | Chr2: 172085611-172088611 | CYBRD1, Chr2: 172085611-172088611 |
| GEMIN4 | Chr17: 600751-603751 | GEMIN4, Chr17: 600751-603751 |
| EPB41L4A | Chr5: 111781409-111784409 | EPB41L4A, Chr5: 111781409-111784409 |
| VIM | Chr10: 17309803-17312803 | VIM, Chr10: 17309803-17312803 |
| HAND1 | Chr5: 153836517-153839517 | HAND1, Chr5: 153836517-153839517 |
| LOC644096 | Chr19: 41176440-41179440 | LOC644096, Chr19: 41176440-41179440 |
| RASGRP2 | Chr11: 64266706-64269706 | RASGRP2, Chr11: 64266706-64269706 |
| FLCN | Chr17: 17079727-17082727 | FLCN, Chr17: 17079727-17082727 |
| HDAC3 | Chr5: 140995107-140998107 | HDAC3, Chr5: 140995107-140998107 |
| NEFM | Chr8: 24826859-24829859 | NEFM, Chr8: 24826859-24829859 |
| DRD1 | Chr5: 174802269-174805269 | DRD1, Chr5: 174802269-174805269 |
| C17orf64 | Chr17: 55853154-55856154 | C17orf64, Chr17: 55853154-55856154 |
| TRIB1 | Chr8: 126510244-126513244 | TRIB1, Chr8: 126510244-126513244 |
| RGS9BP | Chr19: 37856652-37859652 | RGS9BP, Chr19: 37856652-37859652 |
| ARID3A | Chr19: 875536-878536 | ARID3A, Chr19: 875536-878536 |
| SOX1 | Chr13: 111768413-111771413 | SOX1, Chr13: 111768413-111771413 |
| MN1 | Chr22: 26525986-26528986 | MN1, Chr22: 26525986-26528986 |
| POU3F1 | Chr1: 38283537-38286537 | POU3F1, Chr1: 38283537-38286537 |
| ONECUT2 | Chr18: 53252414-53255414 | ONECUT2, Chr18: 53252414-53255414 |
| FAM46A | Chr6: 82517647-82520647 | FAM46A, Chr6: 82517647-82520647 |
| RTBDN | Chr19: 12805730-12808730 | RTBDN, Chr19: 12805730-12808730 |
| KCNQ5 | Chr6: 73387055-73390055 | KCNQ5, Chr6: 73387055-73390055 |
| TRHDE | Chr12: 70951229-70954229 | TRHDE, Chr12: 70951229-70954229 |
| F2R | Chr5: 76046123-76049123 | F2R, Chr5: 76046123-76049123 |
| ABO | Chr9: 135138951-135141951 | ABO, Chr9: 135138951-135141951 |
| ARF1 | Chr1: 226335973-226338973 | ARF1, Chr1: 226335973-226338973 |
| HOXD13 | Chr2: 176664277-176667277 | HOXD13, Chr2: 176664277-176667277 |
| ARHGAP26 | Chr5: 142128975-142131975 | ARHGAP26, Chr5: 142128975-142131975 |
| SMCR8 | Chr17: 18157818-18160818 | SMCR8, Chr17: 18157818-18160818 |
| HM13 | Chr20: 29564401-29567401 | HM13, Chr20: 29564401-29567401 |
| BVES | Chr6: 105689736-105692736 | BVES, Chr6: 105689736-105692736 |
| ECHDC3 | Chr10: 11822861-11825861 | ECHDC3, Chr10: 11822861-11825861 |
| B3GAT1 | Chr11: 133785522-133788522 | B3GAT1, Chr11: 133785522-133788522 |
| SLC6A2 | Chr16: 54246556-54249556 | SLC6A2, Chr16: 54246556-54249556 |
| TPM2 | Chr9: 35678553-35681553 | TPM2, Chr9: 35678553-35681553 |
| CDCA8 | Chr1: 37929245-37932245 | CDCA8, Chr1: 37929245-37932245 |
| KIAA1614 | Chr1: 179147435-179150435 | KIAA1614, Chr1: 179147435-179150435 |
| RASL10B | Chr17: 31081370-31084370 | RASL10B, Chr17: 31081370-31084370 |
| CD3EAP | Chr19: 50599806-50602806 | CD3EAP, Chr19: 50599806-50602806 |
| CCDC136 | Chr7: 128217834-128220834 | CCDC136, Chr7: 128217834-128220834 |
| SLC8A2 | Chr19: 52665434-52668434 | SLC8A2, Chr19: 52665434-52668434 |
| EBF1 | Chr5: 158457866-158460866 | EBF1, Chr5: 158457866-158460866 |
| HSPA2 | Chr14: 64075438-64078438 | HSPA2, Chr14: 64075438-64078438 |
| GFOD1 | Chr6: 13594266-13597266 | GFOD1, Chr6: 13594266-13597266 |
| CDH2 | Chr18: 24009689-24012689 | CDH2, Chr18: 24009689-24012689 |
| FOXD3 | Chr1: 63559817-63562817 | FOXD3, Chr1: 63559817-63562817 |
| NGEF | Chr2: 233499605-233502605 | NGEF, Chr2: 233499605-233502605 |
| SFMBT1 | Chr3: 53053610-53056610 | SFMBT1, Chr3: 53053610-53056610 |
| FBXO31 | Chr16: 85973364-85976364 | FBXO31, Chr16: 85973364-85976364 |
| SIRPA | Chr20: 1821924-1824924 | SIRPA, Chr20: 1821924-1824924 |
| FOXF2 | Chr6: 1333567-1336567 | FOXF2, Chr6: 1333567-1336567 |
| FOXA2 | Chr20: 22511601-22514601 | FOXA2, Chr20: 22511601-22514601 |
| HOXA10 | Chr7: 27184901-27187901 | HOXA10, Chr7: 27184901-27187901 |
| GALR2 | Chr17: 71580986-71583986 | GALR2, Chr17: 71580986-71583986 |
| ING5 | Chr2: 242288628-242291628 | ING5, Chr2: 242288628-242291628 |
| GPR158 | Chr10: 25502795-25505795 | GPR158, Chr10: 25502795-25505795 |
| B4GALNT3 | Chr12: 438303-441303 | B4GALNT3, Chr12: 438303-441303 |
| OTOP1 | Chr4: 4278022-4281022 | OTOP1, Chr4: 4278022-4281022 |
| ADNP | Chr20: 48979434-48982434 | ADNP, Chr20: 48979434-48982434 |
| VAV3 | Chr1: 108307568-108310568 | VAV3, Chr1: 108307568-108310568 |
| CH25H | Chr10: 90955551-90958551 | CH25H, Chr10: 90955551-90958551 |
| HTR1B | Chr6: 78228339-78231339 | HTR1B, Chr6: 78228339-78231339 |
| TBX20 | Chr7: 35258267-35261267 | TBX20, Chr7: 35258267-35261267 |
| GJA3 | Chr13: 19631683-19634683 | GJA3, Chr13: 19631683-19634683 |
| TBX3 | Chr12: 113604852-113607852 | TBX3, Chr12: 113604852-113607852 |
| TET2 | Chr4: 106285891-106288891 | TET2, Chr4: 106285891-106288891 |
| MAGI2 | Chr7: 78919326-78922326 | MAGI2, Chr7: 78919326-78922326 |
| RAB23 | Chr6: 57192716-57195716 | RAB23, Chr6: 57192716-57195716 |
| MEIS2 | Chr15: 35176295-35179295 | MEIS2, Chr15: 35176295-35179295 |
| TACR1 | Chr2: 75278191-75281191 | TACR1, Chr2: 75278191-75281191 |
| KCNG3 | Chr2: 42573241-42576241 | KCNG3, Chr2: 42573241-42576241 |
| SEMA5B | Chr3: 124227766-124230766 | SEMA5B, Chr3: 124227766-124230766 |
| PDE4C | Chr19: 18194803-18197803 | PDE4C, Chr19: 18194803-18197803 |
| CABLES1 | Chr18: 18968224-18971224 | CABLES1, Chr18: 18968224-18971224 |
| SERPINB9 | Chr6: 2847006-2850006 | SERPINB9, Chr6: 2847006-2850006 |
| HNRNPH1 | Chr5: 178981828-178984828 | HNRNPH1, Chr5: 178981828-178984828 |
| ZIC3 | Chr23: 136474511-136477511 | ZIC3, Chr23: 136474511-136477511 |
| DTX4 | Chr11: 58694887-58697887 | DTX4, Chr11: 58694887-58697887 |
| POFUT2 | Chr21: 45530739-45533739 | POFUT2, Chr21: 45530739-45533739 |
| SLBP | Chr4: 1682328-1685328 | SLBP, Chr4: 1682328-1685328 |
| PLSCR3 | Chr17: 7237067-7240067 | PLSCR3, Chr17: 7237067-7240067 |
| ARHGEF7 | Chr13: 110564124-110567124 | ARHGEF7, Chr13: 110564124-110567124 |
| ZMYM5 | Chr13: 19334273-19337273 | ZMYM5, Chr13: 19334273-19337273 |
| MYCN | Chr2: 15996633-15999633 | MYCN, Chr2: 15996633-15999633 |
| BMPER | Chr7: 33909547-33912547 | BMPER, Chr7: 33909547-33912547 |
| CAMK1D | Chr10: 12430088-12433088 | CAMK1D, Chr10: 12430088-12433088 |
| CPLX1 | Chr4: 808445-811445 | CPLX1, Chr4: 808445-811445 |
| TMTC4 | Chr13: 100123604-100126604 | TMTC4, Chr13: 100123604-100126604 |
| ADFP | Chr9: 19116073-19119073 | ADFP, Chr9: 19116073-19119073 |
| BAMBI | Chr10: 29004929-29007929 | BAMBI, Chr10: 29004929-29007929 |
| HERC5 | Chr4: 89595790-89598790 | HERC5, Chr4: 89595790-89598790 |
| SOBP | Chr6: 107916509-107919509 | SOBP, Chr6: 107916509-107919509 |
| NPAS3 | Chr14: 32476773-32479773 | NPAS3, Chr14: 32476773-32479773 |
| ZNF655 | Chr7: 98992883-98995883 | ZNF655, Chr7: 98992883-98995883 |
| CMTM8 | Chr3: 32253674-32256674 | CMTM8, Chr3: 32253674-32256674 |
| UCP2 | Chr11: 73370037-73373037 | UCP2, Chr11: 73370037-73373037 |
| IAH1 | Chr2: 9530620-9533620 | IAH1, Chr2: 9530620-9533620 |
| NDRG2 | Chr14: 20561604-20564604 | NDRG2, Chr14: 20561604-20564604 |
| GDF6 | Chr8: 97240696-97243696 | GDF6, Chr8: 97240696-97243696 |
| CCDC144A | Chr17: 16532863-16535863 | CCDC144A, Chr17: 16532863-16535863 |
| CACNG6 | Chr19: 59185853-59188853 | CACNG6, Chr19: 59185853-59188853 |
| LAMP3 | Chr3: 184361861-184364861 | LAMP3, Chr3: 184361861-184364861 |
| WNT6 | Chr2: 219431289-219434289 | WNT6, Chr2: 219431289-219434289 |
| RNF165 | Chr18: 42166684-42169684 | RNF165, Chr18: 42166684-42169684 |
| ZBTB4 | Chr17: 7322168-7325168 | ZBTB4, Chr17: 7322168-7325168 |
| TIMM22 | Chr17: 845606-848606 | TIMM22, Chr17: 845606-848606 |
| TACR1 | Chr2: 75278281-75281281 | TACR1, Chr2: 75278281-75281281 |
| HIST3H2A | Chr1: 226710683-226713683 | HIST3H2A, Chr1: 226710683-226713683 |
| RGMA | Chr15: 91431937-91434937 | RGMA, Chr15: 91431937-91434937 |
| CUL4A | Chr13: 112909586-112912586 | CUL4A, Chr13: 112909586-112912586 |
| TCF7L1 | Chr2: 85212744-85215744 | TCF7L1, Chr2: 85212744-85215744 |
| HCN4 | Chr15: 71446730-71449730 | HCN4, Chr15: 71446730-71449730 |
| MNX1 | Chr7: 156494608-156497608 | MNX1, Chr7: 156494608-156497608 |
| TLX2 | Chr2: 74593618-74596618 | TLX2, Chr2: 74593618-74596618 |
| KIT | Chr4: 55217351-55220351 | KIT, Chr4: 55217351-55220351 |
| EN2 | Chr7: 154942084-154945084 | EN2, Chr7: 154942084-154945084 |
| FADS2 | Chr11: 61350788-61353788 | FADS2, Chr11: 61350788-61353788 |
| PRKCH | Chr14: 60856767-60859767 | PRKCH, Chr14: 60856767-60859767 |
| ADCY1 | Chr7: 45579149-45582149 | ADCY1, Chr7: 45579149-45582149 |
| TMEM132D | Chr12: 128952665-128955665 | TMEM132D, Chr12: 128952665-128955665 |
| GRIN2A | Chr16: 10182612-10185612 | GRIN2A, Chr16: 10182612-10185612 |
| DOK6 | Chr18: 65217770-65220770 | DOK6, Chr18: 65217770-65220770 |
| KCNMA1 | Chr10: 79066083-79069083 | KCNMA1, Chr10: 79066083-79069083 |
| MIER2 | Chr19: 294291-297291 | MIER2, Chr19: 294291-297291 |
| SCUBE1 | Chr22: 42067799-42070799 | SCUBE1, Chr22: 42067799-42070799 |
| LHX1 | Chr17: 32367111-32370111 | LHX1, Chr17: 32367111-32370111 |
| TMEM64 | Chr8: 91725612-91728612 | TMEM64, Chr8: 91725612-91728612 |
| TOP3A | Chr17: 18157546-18160546 | TOP3A, Chr17: 18157546-18160546 |
| MLLT10 | Chr10: 21862079-21865079 | MLLT10, Chr10: 21862079-21865079 |
| ACPL2 | Chr3: 142431871-142434871 | ACPL2, Chr3: 142431871-142434871 |
| NPAS3 | Chr14: 32476709-32479709 | NPAS3, Chr14: 32476709-32479709 |
| STOM | Chr9: 123170866-123173866 | STOM, Chr9: 123170866-123173866 |
| WDR82 | Chr3: 52286199-52289199 | WDR82, Chr3: 52286199-52289199 |
| C12orf62 | Chr12: 48790666-48793666 | C12orf62, Chr12: 48790666-48793666 |
| THAP11 | Chr16: 66432213-66435213 | THAP11, Chr16: 66432213-66435213 |
| GALNT17 | Chr4: 172969649-172972649 | GALNT17, Chr4: 172969649-172972649 |
| LAMC1 | Chr1: 181257717-181260717 | LAMC1, Chr1: 181257717-181260717 |
| CPNE5 | Chr6: 36913698-36916698 | CPNE5, Chr6: 36913698-36916698 |
| TBL1XR1 | Chr3: 178396242-178399242 | TBL1XR1, Chr3: 178396242-178399242 |
| DMRTA2 | Chr1: 50660229-50663229 | DMRTA2, Chr1: 50660229-50663229 |
| HAAO | Chr2: 42871755-42874755 | HAAO, Chr2: 42871755-42874755 |
| ZFP90 | Chr16: 67129661-67132661 | ZFP90, Chr16: 67129661-67132661 |
| BTG4 | Chr11: 110886774-110889774 | BTG4, Chr11: 110886774-110889774 |
| UCHL5 | Chr1: 191293644-191296644 | UCHL5, Chr1: 191293644-191296644 |
| SDCBP2 | Chr20: 1256338-1259338 | SDCBP2, Chr20: 1256338-1259338 |
| UBXD3 | Chr1: 20383664-20386664 | UBXD3, Chr1: 20383664-20386664 |
| FEZF2 | Chr3: 62332730-62335730 | FEZF2, Chr3: 62332730-62335730 |
| HAS3 | Chr16: 67696160-67699160 | HAS3, Chr16: 67696160-67699160 |
| SOCS1 | Chr16: 11256040-11259040 | SOCS1, Chr16: 11256040-11259040 |
| GABRB3 | Chr15: 24567844-24570844 | GABRB3, Chr15: 24567844-24570844 |
| EGR3 | Chr8: 22605260-22608260 | EGR3, Chr8: 22605260-22608260 |
| GALR1 | Chr18: 73089495-73092495 | GALR1, Chr18: 73089495-73092495 |
| STC2 | Chr5: 172687612-172690612 | STC2, Chr5: 172687612-172690612 |
| EMX2 | Chr10: 119290445-119293445 | EMX2, Chr10: 119290445-119293445 |
| C14orf169 | Chr14: 73025896-73028896 | C14orf169, Chr14: 73025896-73028896 |
| PTPRT | Chr20: 41250471-41253471 | PTPRT, Chr20: 41250471-41253471 |
| TRH | Chr3: 131174752-131177752 | TRH, Chr3: 131174752-131177752 |
| CENTG3 | Chr7: 150413258-150416258 | CENTG3, Chr7: 150413258-150416258 |
| SYT9 | Chr11: 7228256-7231256 | SYT9, Chr11: 7228256-7231256 |
| RBP1 | Chr3: 140739680-140742680 | RBP1, Chr3: 140739680-140742680 |
| KL | Chr13: 32487070-32490070 | KL, Chr13: 32487070-32490070 |
| WDR6 | Chr3: 49018329-49021329 | WDR6, Chr3: 49018329-49021329 |
| DPYSL3 | Chr5: 146811953-146814953 | DPYSL3, Chr5: 146811953-146814953 |
| DNM3 | Chr1: 170075760-170078760 | DNM3, Chr1: 170075760-170078760 |
| CORO1C | Chr12: 107647924-107650924 | CORO1C, Chr12: 107647924-107650924 |
| DLX1 | Chr2: 172656953-172659953 | DLX1, Chr2: 172656953-172659953 |
| ST3GAL3 | Chr1: 43944304-43947304 | ST3GAL3, Chr1: 43944304-43947304 |
| ALDH3A2 | Chr17: 19491155-19494155 | ALDH3A2, Chr17: 19491155-19494155 |
| STK32B | Chr4: 5102927-5105927 | STK32B, Chr4: 5102927-5105927 |
| MGAT5B | Chr17: 72374892-72377892 | MGAT5B, Chr17: 72374892-72377892 |
| DNAH10 | Chr12: 122811494-122814494 | DNAH10, Chr12: 122811494-122814494 |
| SERHL | Chr22: 41225039-41228039 | SERHL, Chr22: 41225039-41228039 |
| TIMM17A | Chr1: 200189770-200192770 | TIMM17A, Chr1: 200189770-200192770 |
| DGKZ | Chr11: 46309814-46312814 | DGKZ, Chr11: 46309814-46312814 |
| SAMM50 | Chr22: 42681133-42684133 | SAMM50, Chr22: 42681133-42684133 |
| FMNL1 | Chr17: 40653574-40656574 | FMNL1, Chr17: 40653574-40656574 |
| C1orf106 | Chr1: 199125791-199128791 | C1orf106, Chr1: 199125791-199128791 |
| NRP2 | Chr2: 206253968-206256968 | NRP2, Chr2: 206253968-206256968 |
| ZFP28 | Chr19: 61740628-61743628 | ZFP28, Chr19: 61740628-61743628 |
| RIMS4 | Chr20: 42870826-42873826 | RIMS4, Chr20: 42870826-42873826 |
| FAM46C | Chr1: 117948626-117951626 | FAM46C, Chr1: 117948626-117951626 |
| ZNF454 | Chr5: 178299329-178302329 | ZNF454, Chr5: 178299329-178302329 |
| EFCAB1 | Chr8: 49808844-49811844 | EFCAB1, Chr8: 49808844-49811844 |
| UGT3A2 | Chr5: 36101241-36104241 | UGT3A2, Chr5: 36101241-36104241 |
| CCDC134 | Chr22: 40525123-40528123 | CCDC134, Chr22: 40525123-40528123 |
| AKAP2 | Chr9: 111849296-111852296 | AKAP2, Chr9: 111849296-111852296 |
| IRF4 | Chr6: 335251-338251 | IRF4, Chr6: 335251-338251 |
| RUNX2 | Chr6: 45496391-45499391 | RUNX2, Chr6: 45496391-45499391 |
| CACNG4 | Chr17: 62389974-62392974 | CACNG4, Chr17: 62389974-62392974 |
| LHX2 | Chr9: 125812209-125815209 | LHX2, Chr9: 125812209-125815209 |
| UST | Chr6: 149108656-149111656 | UST, Chr6: 149108656-149111656 |
| ARNTL | Chr11: 13254400-13257400 | ARNTL, Chr11: 13254400-13257400 |
| CHST11 | Chr12: 103373407-103376407 | CHST11, Chr12: 103373407-103376407 |
| FOXL2 | Chr3: 140146991-140149991 | FOXL2, Chr3: 140146991-140149991 |
| PPARGC1B | Chr5: 149088556-149091556 | PPARGC1B, Chr5: 149088556-149091556 |
| RRBP1 | Chr20: 17609428-17612428 | RRBP1, Chr20: 17609428-17612428 |
| CBS | Chr21: 43367993-43370993 | CBS, Chr21: 43367993-43370993 |
| C6orf145 | Chr6: 3695745-3698745 | C6orf145, Chr6: 3695745-3698745 |
| SNAP91 | Chr6: 84473995-84476995 | SNAP91, Chr6: 84473995-84476995 |
| ADORA2B | Chr17: 15787455-15790455 | ADORA2B, Chr17: 15787455-15790455 |
| TDRD10 | Chr1: 152739818-152742818 | TDRD10, Chr1: 152739818-152742818 |
| ARF1 | Chr1: 226335483-226338483 | ARF1, Chr1: 226335483-226338483 |
| HSPA12A | Chr10: 118490575-118493575 | HSPA12A, Chr10: 118490575-118493575 |
| LOC387856 | Chr12: 46862165-46865165 | LOC387856, Chr12: 46862165-46865165 |
| GATA5 | Chr20: 60482921-60485921 | GATA5, Chr20: 60482921-60485921 |
| PTGER4 | Chr5: 40714288-40717288 | PTGER4, Chr5: 40714288-40717288 |
| FAM108B1 | Chr9: 73714468-73717468 | FAM108B1, Chr9: 73714468-73717468 |
| GHR | Chr5: 42458282-42461282 | GHR, Chr5: 42458282-42461282 |
| FAM84A | Chr2: 14688806-14691806 | FAM84A, Chr2: 14688806-14691806 |
| AFAP1L1 | Chr5: 148630150-148633150 | AFAP1L1, Chr5: 148630150-148633150 |
| RIN3 | Chr14: 92048377-92051377 | RIN3, Chr14: 92048377-92051377 |
| RYR2 | Chr1: 235270824-235273824 | RYR2, Chr1: 235270824-235273824 |
| DUSP5 | Chr10: 112246114-112249114 | DUSP5, Chr10: 112246114-112249114 |
| NKX6-1 | Chr4: 85636911-85639911 | NKX6-1, Chr4: 85636911-85639911 |
| STK25 | Chr2: 242095207-242098207 | STK25, Chr2: 242095207-242098207 |
| ADAMTSL5 | Chr19: 1462519-1465519 | ADAMTSL5, Chr19: 1462519-1465519 |
| PTPN5 | Chr11: 18768465-18771465 | PTPN5, Chr11: 18768465-18771465 |
| PHF11 | Chr13: 48966301-48969301 | PHF11, Chr13: 48966301-48969301 |
| KREMEN2 | Chr16: 2952717-2955717 | KREMEN2, Chr16: 2952717-2955717 |
| GPR137B | Chr1: 234370996-234373996 | GPR137B, Chr1: 234370996-234373996 |
| GPRIN1 | Chr5: 175968237-175971237 | GPRIN1, Chr5: 175968237-175971237 |
| GNAZ | Chr22: 21741168-21744168 | GNAZ, Chr22: 21741168-21744168 |
| TCERG1L | Chr10: 132998474-133001474 | TCERG1L, Chr10: 132998474-133001474 |
| DMRT2 | Chr9: 1039100-1042100 | DMRT2, Chr9: 1039100-1042100 |
| ARHGEF3 | Chr3: 57086876-57089876 | ARHGEF3, Chr3: 57086876-57089876 |
| SLAIN2 | Chr4: 48036869-48039869 | SLAIN2, Chr4: 48036869-48039869 |
| SYNGR1 | Chr22: 38074399-38077399 | SYNGR1, Chr22: 38074399-38077399 |
| TRPC1 | Chr3: 143924455-143927455 | TRPC1, Chr3: 143924455-143927455 |
| FAM150A | Chr8: 53639065-53642065 | FAM150A, Chr8: 53639065-53642065 |
| NIPSNAP3B | Chr9: 106564771-106567771 | NIPSNAP3B, Chr9: 106564771-106567771 |
| HAP1 | Chr17: 37142924-37145924 | HAP1, Chr17: 37142924-37145924 |
| AP3M2 | Chr8: 42128260-42131260 | AP3M2, Chr8: 42128260-42131260 |
| ZNF132 | Chr19: 63641901-63644901 | ZNF132, Chr19: 63641901-63644901 |
| CIDEA | Chr18: 12243127-12246127 | CIDEA, Chr18: 12243127-12246127 |
| CAMK4 | Chr5: 110586480-110589480 | CAMK4, Chr5: 110586480-110589480 |
| SLC22A17 | Chr14: 22889819-22892819 | SLC22A17, Chr14: 22889819-22892819 |
| GTSE1 | Chr22: 45069975-45072975 | GTSE1, Chr22: 45069975-45072975 |
| COCH | Chr14: 30412015-30415015 | COCH, Chr14: 30412015-30415015 |
| TMEM37 | Chr2: 119904415-119907415 | TMEM37, Chr2: 119904415-119907415 |
| C1orf113 | Chr1: 36543804-36546804 | C1orf113, Chr1: 36543804-36546804 |
| RLBP1L2 | Chr6: 123357780-123360780 | RLBP1L2, Chr6: 123357780-123360780 |
| DYDC1 | Chr10: 82104980-82107980 | DYDC1, Chr10: 82104980-82107980 |
| TRIM59 | Chr3: 161648820-161651820 | TRIM59, Chr3: 161648820-161651820 |
| DMGDH | Chr5: 78399705-78402705 | DMGDH, Chr5: 78399705-78402705 |
| LEP | Chr7: 127667066-127670066 | LEP, Chr7: 127667066-127670066 |
| TSPAN11 | Chr12: 30969604-30972604 | TSPAN11, Chr12: 30969604-30972604 |
| HIST1H2BJ | Chr6: 27207054-27210054 | HIST1H2BJ, Chr6: 27207054-27210054 |
| NPY2R | Chr4: 156347730-156350730 | NPY2R, Chr4: 156347730-156350730 |
| KIAA0644 | Chr7: 28963054-28966054 | KIAA0644, Chr7: 28963054-28966054 |
| ZC3HAV1L | Chr7: 138369815-138372815 | ZC3HAV1L, Chr7: 138369815-138372815 |
| GLS2 | Chr12: 55166948-55169948 | GLS2, Chr12: 55166948-55169948 |
| OLIG1 | Chr21: 33362942-33365942 | OLIG1, Chr21: 33362942-33365942 |
| TBC1D4 | Chr13: 74952751-74955751 | TBC1D4, Chr13: 74952751-74955751 |
| GABBR2 | Chr9: 100509800-100512800 | GABBR2, Chr9: 100509800-100512800 |
| KLF2 | Chr19: 16295150-16298150 | KLF2, Chr19: 16295150-16298150 |
| GPR150 | Chr5: 94980235-94983235 | GPR150, Chr5: 94980235-94983235 |
| SFRP1 | Chr8: 41284637-41287637 | SFRP1, Chr8: 41284637-41287637 |
| 9-Sep | Chr17: 72879366-72882366 | 40795, Chr17: 72879366-72882366 |
| KIAA1217 | Chr10: 24022180-24025180 | KIAA1217, Chr10: 24022180-24025180 |
| SHROOM4 | Chr23: 50572284-50575284 | SHROOM4, Chr23: 50572284-50575284 |
| CYS1 | Chr2: 10136489-10139489 | CYS1, Chr2: 10136489-10139489 |
| HS3ST4 | Chr16: 25609347-25612347 | HS3ST4, Chr16: 25609347-25612347 |
| EYA4 | Chr6: 133602687-133605687 | EYA4, Chr6: 133602687-133605687 |
| FAM20A | Chr17: 64107190-64110190 | FAM20A, Chr17: 64107190-64110190 |
| HOXA4 | Chr7: 27135424-27138424 | HOXA4, Chr7: 27135424-27138424 |
| DUS1L | Chr17: 77615469-77618469 | DUS1L, Chr17: 77615469-77618469 |
| NANOS1 | Chr10: 120777717-120780717 | NANOS1, Chr10: 120777717-120780717 |
| GPR37 | Chr7: 124191417-124194417 | GPR37, Chr7: 124191417-124194417 |
| MLNR | Chr13: 48690974-48693974 | MLNR, Chr13: 48690974-48693974 |
| PAQR8 | Chr6: 52333384-52336384 | PAQR8, Chr6: 52333384-52336384 |
| CALN1 | Chr7: 71438644-71441644 | CALN1, Chr7: 71438644-71441644 |
| ADAMTSL3 | Chr15: 82112341-82115341 | ADAMTSL3, Chr15: 82112341-82115341 |
| CRTC3 | Chr15: 88872701-88875701 | CRTC3, Chr15: 88872701-88875701 |
| NKX3-2 | Chr4: 13153712-13156712 | NKX3-2, Chr4: 13153712-13156712 |
| MAP6D1 | Chr3: 185024559-185027559 | MAP6D1, Chr3: 185024559-185027559 |
| HOXD11 | Chr2: 176678829-176681829 | HOXD11, Chr2: 176678829-176681829 |
| SEC14L4 | Chr22: 29230182-29233182 | SEC14L4, Chr22: 29230182-29233182 |
| CYB5R3 | Chr22: 41373849-41376849 | CYB5R3, Chr22: 41373849-41376849 |
| ADD3 | Chr10: 111756200-111759200 | ADD3, Chr10: 111756200-111759200 |
| KCNG1 | Chr20: 49071582-49074582 | KCNG1, Chr20: 49071582-49074582 |
| KCNJ12 | Chr17: 21218791-21221791 | KCNJ12, Chr17: 21218791-21221791 |
| CLDN10 | Chr13: 95001460-95004460 | CLDN10, Chr13: 95001460-95004460 |
| DMC1 | Chr22: 37294635-37297635 | DMC1, Chr22: 37294635-37297635 |
| ITGA9 | Chr3: 37467316-37470316 | ITGA9, Chr3: 37467316-37470316 |
| NT5DC3 | Chr12: 102757414-102760414 | NT5DC3, Chr12: 102757414-102760414 |
| FAM83F | Chr22: 38719398-38722398 | FAM83F, Chr22: 38719398-38722398 |
| TJP2 | Chr9: 70977408-70980408 | TJP2, Chr9: 70977408-70980408 |
| RASL11B | Chr4: 53421751-53424751 | RASL11B, Chr4: 53421751-53424751 |
| ELK1 | Chr23: 47393447-47396447 | ELK1, Chr23: 47393447-47396447 |
| AMPD3 | Chr11: 10427299-10430299 | AMPD3, Chr11: 10427299-10430299 |
| RGS22 | Chr8: 101186020-101189020 | RGS22, Chr8: 101186020-101189020 |
| SLC46A3 | Chr13: 28189462-28192462 | SLC46A3, Chr13: 28189462-28192462 |
| VWA3B | Chr2: 98068526-98071526 | VWA3B, Chr2: 98068526-98071526 |
| IRAK2 | Chr3: 10180062-10183062 | IRAK2, Chr3: 10180062-10183062 |
| TROVE2 | Chr1: 191294079-191297079 | TROVE2, Chr1: 191294079-191297079 |
| XK | Chr23: 37428551-37431551 | XK, Chr23: 37428551-37431551 |
| SLC2A14 | Chr12: 7915262-7918262 | SLC2A14, Chr12: 7915262-7918262 |
| PRUNE2 | Chr9: 78709323-78712323 | PRUNE2, Chr9: 78709323-78712323 |
| P2RY2 | Chr11: 72605649-72608649 | P2RY2, Chr11: 72605649-72608649 |
| DEPDC7 | Chr11: 32992803-32995803 | DEPDC7, Chr11: 32992803-32995803 |
| MT1E | Chr16: 55215585-55218585 | MT1E, Chr16: 55215585-55218585 |
| RSPO2 | Chr8: 109163242-109166242 | RSPO2, Chr8: 109163242-109166242 |
| C4orf31 | Chr4: 122211623-122214623 | C4orf31, Chr4: 122211623-122214623 |
| SOX17 | Chr8: 55531547-55534547 | SOX17, Chr8: 55531547-55534547 |
| DLX2 | Chr2: 172674224-172677224 | DLX2, Chr2: 172674224-172677224 |
| NPR1 | Chr1: 151916287-151919287 | NPR1, Chr1: 151916287-151919287 |
| FZD1 | Chr7: 90730218-90733218 | FZD1, Chr7: 90730218-90733218 |
| ABCA1 | Chr9: 106728757-106731757 | ABCA1, Chr9: 106728757-106731757 |
| CECR6 | Chr22: 15980643-15983643 | CECR6, Chr22: 15980643-15983643 |
| SIRPA | Chr20: 1821312-1824312 | SIRPA, Chr20: 1821312-1824312 |
| NPBWR1 | Chr8: 54013520-54016520 | NPBWR1, Chr8: 54013520-54016520 |
| FOXE1 | Chr9: 99653857-99656857 | FOXE1, Chr9: 99653857-99656857 |
| FJX1 | Chr11: 35595130-35598130 | FJX1, Chr11: 35595130-35598130 |
| FSCN1 | Chr7: 5597479-5600479 | FSCN1, Chr7: 5597479-5600479 |
| RPRML | Chr17: 42410113-42413113 | RPRML, Chr17: 42410113-42413113 |
| GYG2 | Chr23: 2755362-2758362 | GYG2, Chr23: 2755362-2758362 |
| LHX6 | Chr9: 124029340-124032340 | LHX6, Chr9: 124029340-124032340 |
| LRRTM1 | Chr2: 80383498-80386498 | LRRTM1, Chr2: 80383498-80386498 |
| GDF11 | Chr12: 54421833-54424833 | GDF11, Chr12: 54421833-54424833 |
| CA10 | Chr17: 47589631-47592631 | CA10, Chr17: 47589631-47592631 |
| TUBA4A | Chr2: 219825382-219828382 | TUBA4A, Chr2: 219825382-219828382 |
| INA | Chr10: 105025409-105028409 | INA, Chr10: 105025409-105028409 |
| DCC | Chr18: 48119068-48122068 | DCC, Chr18: 48119068-48122068 |
| GRIA2 | Chr4: 158359769-158362769 | GRIA2, Chr4: 158359769-158362769 |
| JAKMIP1 | Chr4: 6251719-6254719 | JAKMIP1, Chr4: 6251719-6254719 |
| ZNF655 | Chr7: 98992480-98995480 | ZNF655, Chr7: 98992480-98995480 |
| KLF14 | Chr7: 130067900-130070900 | KLF14, Chr7: 130067900-130070900 |
| CCDC74A | Chr2: 132000461-132003461 | CCDC74A, Chr2: 132000461-132003461 |
| GRIA2 | Chr4: 158359685-158362685 | GRIA2, Chr4: 158359685-158362685 |
| PAK1 | Chr11: 76861256-76864256 | PAK1, Chr11: 76861256-76864256 |
| SPHKAP | Chr2: 228753086-228756086 | SPHKAP, Chr2: 228753086-228756086 |
| HTR7 | Chr10: 92606151-92609151 | HTR7, Chr10: 92606151-92609151 |
| ESR1 | Chr6: 152169006-152172006 | ESR1, Chr6: 152169006-152172006 |
| AXUD1 | Chr3: 39168605-39171605 | AXUD1, Chr3: 39168605-39171605 |
| NID1 | Chr1: 234293604-234296604 | NID1, Chr1: 234293604-234296604 |
| SCN5A | Chr3: 38664667-38667667 | SCN5A, Chr3: 38664667-38667667 |
| C7orf57 | Chr7: 48040141-48043141 | C7orf57, Chr7: 48040141-48043141 |
| FLRT2 | Chr14: 85064740-85067740 | FLRT2, Chr14: 85064740-85067740 |
| DIDO1 | Chr20: 61026790-61029790 | DIDO1, Chr20: 61026790-61029790 |
| PPARG | Chr3: 12302848-12305848 | PPARG, Chr3: 12302848-12305848 |
| LAMA3 | Chr18: 19522059-19525059 | LAMA3, Chr18: 19522059-19525059 |
| SRrp35 | Chr6: 89883019-89886019 | SRrp35, Chr6: 89883019-89886019 |
| WDR85 | Chr9: 139591708-139594708 | WDR85, Chr9: 139591708-139594708 |
| RHOQ | Chr2: 46621870-46624870 | RHOQ, Chr2: 46621870-46624870 |
| STMN1 | Chr1: 26103731-26106731 | STMN1, Chr1: 26103731-26106731 |
| PCDHGB6 | Chr5: 140766453-140769453 | PCDHGB6, Chr5: 140766453-140769453 |
| EVC2 | Chr4: 5759695-5762695 | EVC2, Chr4: 5759695-5762695 |
| LBX2 | Chr2: 74582451-74585451 | LBX2, Chr2: 74582451-74585451 |
| GRAMD2 | Chr15: 70275680-70278680 | GRAMD2, Chr15: 70275680-70278680 |
| RAB33A | Chr23: 129131953-129134953 | RAB33A, Chr23: 129131953-129134953 |
| THAP7 | Chr22: 19684904-19687904 | THAP7, Chr22: 19684904-19687904 |
| LRRK1 | Chr15: 99275482-99278482 | LRRK1, Chr15: 99275482-99278482 |
| ANKRD34B | Chr5: 79900354-79903354 | ANKRD34B, Chr5: 79900354-79903354 |
| PALM | Chr19: 658452-661452 | PALM, Chr19: 658452-661452 |
| SLC6A11 | Chr3: 10831416-10834416 | SLC6A11, Chr3: 10831416-10834416 |
| FAM44A | Chr4: 13236926-13239926 | FAM44A, Chr4: 13236926-13239926 |
| GABRE | Chr23: 150892307-150895307 | GABRE, Chr23: 150892307-150895307 |
| ACTN2 | Chr1: 234914892-234917892 | ACTN2, Chr1: 234914892-234917892 |
| DYDC2 | Chr10: 82105037-82108037 | DYDC2, Chr10: 82105037-82108037 |
| EIF5A2 | Chr3: 172107620-172110620 | EIF5A2, Chr3: 172107620-172110620 |
| COL14A1 | Chr8: 121205032-121208032 | COL14A1, Chr8: 121205032-121208032 |
| DGKI | Chr7: 137180649-137183649 | DGKI, Chr7: 137180649-137183649 |
| LPAR1 | Chr9: 112838686-112841686 | LPAR1, Chr9: 112838686-112841686 |
| ZYX | Chr7: 142786981-142789981 | ZYX, Chr7: 142786981-142789981 |
| BHMT2 | Chr5: 78399838-78402838 | BHMT2, Chr5: 78399838-78402838 |
| CTSF | Chr11: 66091123-66094123 | CTSF, Chr11: 66091123-66094123 |
| SPAG6 | Chr10: 22672904-22675904 | SPAG6, Chr10: 22672904-22675904 |
| TDRD5 | Chr1: 177826147-177829147 | TDRD5, Chr1: 177826147-177829147 |
| C20orf24 | Chr20: 34666080-34669080 | C20orf24, Chr20: 34666080-34669080 |
| APBB1 | Chr11: 6395376-6398376 | APBB1, Chr11: 6395376-6398376 |
| TRIT1 | Chr1: 40120264-40123264 | TRIT1, Chr1: 40120264-40123264 |
| STAC | Chr3: 36395600-36398600 | STAC, Chr3: 36395600-36398600 |
| TSPAN2 | Chr1: 115432138-115435138 | TSPAN2, Chr1: 115432138-115435138 |
| PCDP1 | Chr2: 120017016-120020016 | PCDP1, Chr2: 120017016-120020016 |
| MCOLN3 | Chr1: 85285257-85288257 | MCOLN3, Chr1: 85285257-85288257 |
| UCP1 | Chr4: 141707957-141710957 | UCP1, Chr4: 141707957-141710957 |
| GSN | Chr9: 123100399-123103399 | GSN, Chr9: 123100399-123103399 |
| CCDC88A | Chr2: 55498967-55501967 | CCDC88A, Chr2: 55498967-55501967 |
| FLJ40504 | Chr17: 23656644-23659644 | FLJ40504, Chr17: 23656644-23659644 |
| CCDC67 | Chr11: 92702030-92705030 | CCDC67, Chr11: 92702030-92705030 |
| ST6GALNAC3 | Chr1: 76311491-76314491 | ST6GALNAC3, Chr1: 76311491-76314491 |
| TMEM106A | Chr17: 38717919-38720919 | TMEM106A, Chr17: 38717919-38720919 |
| LRAT | Chr4: 155883112-155886112 | LRAT, Chr4: 155883112-155886112 |
| NKX2-3 | Chr10: 101281179-101284179 | NKX2-3, Chr10: 101281179-101284179 |
| ELOVL3 | Chr10: 103974632-103977632 | ELOVL3, Chr10: 103974632-103977632 |
| IQCA1 | Chr2: 237079331-237082331 | IQCA1, Chr2: 237079331-237082331 |
| APCDD1L | Chr20: 56521855-56524855 | APCDD1L, Chr20: 56521855-56524855 |
| CDKL2 | Chr4: 76773245-76776245 | CDKL2, Chr4: 76773245-76776245 |
| GRIA4 | Chr11: 104985120-104988120 | GRIA4, Chr11: 104985120-104988120 |
| DSC3 | Chr18: 26875279-26878279 | DSC3, Chr18: 26875279-26878279 |
| FMN2 | Chr1: 238320307-238323307 | FMN2, Chr1: 238320307-238323307 |
| KCTD8 | Chr4: 44144081-44147081 | KCTD8, Chr4: 44144081-44147081 |
| FGFR2 | Chr10: 123346462-123349462 | FGFR2, Chr10: 123346462-123349462 |
| POU4F1 | Chr13: 78074196-78077196 | POU4F1, Chr13: 78074196-78077196 |
| PTRF | Chr17: 37827300-37830300 | PTRF, Chr17: 37827300-37830300 |
| NTNG1 | Chr1: 107482767-107485767 | NTNG1, Chr1: 107482767-107485767 |
| SLC8A3 | Chr14: 69724040-69727040 | SLC8A3, Chr14: 69724040-69727040 |
| LAMB1 | Chr7: 107429540-107432540 | LAMB1, Chr7: 107429540-107432540 |
| MAP1B | Chr5: 71437373-71440373 | MAP1B, Chr5: 71437373-71440373 |
| NTF3 | Chr12: 5410040-5413040 | NTF3, Chr12: 5410040-5413040 |
| HOXC10 | Chr12: 52663712-52666712 | HOXC10, Chr12: 52663712-52666712 |
| NCOA2 | Chr8: 71477074-71480074 | NCOA2, Chr8: 71477074-71480074 |
| AMOTL2 | Chr3: 135574596-135577596 | AMOTL2, Chr3: 135574596-135577596 |
| CNOT2 | Chr12: 68921543-68924543 | CNOT2, Chr12: 68921543-68924543 |
| PDX1 | Chr13: 27390667-27393667 | PDX1, Chr13: 27390667-27393667 |
| TMEM22 | Chr3: 138019050-138022050 | TMEM22, Chr3: 138019050-138022050 |
| MLLT10 | Chr10: 21861607-21864607 | MLLT10, Chr10: 21861607-21864607 |
| PRRX1 | Chr1: 168898436-168901436 | PRRX1, Chr1: 168898436-168901436 |
| CDO1 | Chr5: 115178804-115181804 | CDO1, Chr5: 115178804-115181804 |
| FAM62C | Chr3: 139634608-139637608 | FAM62C, Chr3: 139634608-139637608 |
| RASA3 | Chr13: 113914697-113917697 | RASA3, Chr13: 113914697-113917697 |
| RELL2 | Chr5: 140995200-140998200 | RELL2, Chr5: 140995200-140998200 |
| MAD1L1 | Chr7: 2237609-2240609 | MAD1L1, Chr7: 2237609-2240609 |
| SYT10 | Chr12: 33482521-33485521 | SYT10, Chr12: 33482521-33485521 |
| CCBE1 | Chr18: 55514070-55517070 | CCBE1, Chr18: 55514070-55517070 |
| HTR1A | Chr5: 63291802-63294802 | HTR1A, Chr5: 63291802-63294802 |
| PAX6 | Chr11: 31794585-31797585 | PAX6, Chr11: 31794585-31797585 |
| CCNJL | Chr5: 159670651-159673651 | CCNJL, Chr5: 159670651-159673651 |
| EMILIN3 | Chr20: 39427412-39430412 | EMILIN3, Chr20: 39427412-39430412 |
| ATP8A2 | Chr13: 24842708-24845708 | ATP8A2, Chr13: 24842708-24845708 |
| PCDHGB5 | Chr5: 140756378-140759378 | PCDHGB5, Chr5: 140756378-140759378 |
| PCDHGB4 | Chr5: 140746135-140749135 | PCDHGB4, Chr5: 140746135-140749135 |
| CSNK1A1 | Chr5: 148909700-148912700 | CSNK1A1, Chr5: 148909700-148912700 |
| USP44 | Chr12: 94465251-94468251 | USP44, Chr12: 94465251-94468251 |
| NLP | Chr20: 25512653-25515653 | NLP, Chr20: 25512653-25515653 |
| SLC7A5 | Chr16: 86459101-86462101 | SLC7A5, Chr16: 86459101-86462101 |
| GLT1D1 | Chr12: 127902533-127905533 | GLT1D1, Chr12: 127902533-127905533 |
| NT5DC3 | Chr12: 102757605-102760605 | NT5DC3, Chr12: 102757605-102760605 |
| C15orf56 | Chr15: 38330902-38333902 | C15orf56, Chr15: 38330902-38333902 |
| RALYL | Chr8: 85256640-85259640 | RALYL, Chr8: 85256640-85259640 |
| LGALS3 | Chr14: 54664124-54667124 | LGALS3, Chr14: 54664124-54667124 |
| EDNRB | Chr13: 77389467-77392467 | EDNRB, Chr13: 77389467-77392467 |
| SPATA6 | Chr1: 48708932-48711932 | SPATA6, Chr1: 48708932-48711932 |
| C9orf64 | Chr9: 85759983-85762983 | C9orf64, Chr9: 85759983-85762983 |
| CIDEA | Chr18: 12242817-12245817 | CIDEA, Chr18: 12242817-12245817 |
| PDE4D | Chr5: 59223878-59226878 | PDE4D, Chr5: 59223878-59226878 |
| SNTG2 | Chr2: 935054-938054 | SNTG2, Chr2: 935054-938054 |
| C6orf186 | Chr6: 110784668-110787668 | C6orf186, Chr6: 110784668-110787668 |
| CTTNBP2 | Chr7: 117299297-117302297 | CTTNBP2, Chr7: 117299297-117302297 |
| NT5C1A | Chr1: 39908797-39911797 | NT5C1A, Chr1: 39908797-39911797 |
| TXNRD1 | Chr12: 103132188-103135188 | TXNRD1, Chr12: 103132188-103135188 |
| FRMD8 | Chr11: 64909116-64912116 | FRMD8, Chr11: 64909116-64912116 |
| NEK6 | Chr9: 126058569-126061569 | NEK6, Chr9: 126058569-126061569 |
| SARM1 | Chr17: 23721613-23724613 | SARM1, Chr17: 23721613-23724613 |
| TROVE2 | Chr1: 191293875-191296875 | TROVE2, Chr1: 191293875-191296875 |
| WDR33 | Chr2: 128283715-128286715 | WDR33, Chr2: 128283715-128286715 |
| SYK | Chr9: 92602390-92605390 | SYK, Chr9: 92602390-92605390 |
| OAT | Chr10: 126096009-126099009 | OAT, Chr10: 126096009-126099009 |
| GLUD2 | Chr23: 120007642-120010642 | GLUD2, Chr23: 120007642-120010642 |
| ZNF774 | Chr15: 88694980-88697980 | ZNF774, Chr15: 88694980-88697980 |
| P2RY2 | Chr11: 72605491-72608491 | P2RY2, Chr11: 72605491-72608491 |
| CHGA | Chr14: 92457697-92460697 | CHGA, Chr14: 92457697-92460697 |
| HSPB1 | Chr7: 75768358-75771358 | HSPB1, Chr7: 75768358-75771358 |
| PDK4 | Chr7: 95062361-95065361 | PDK4, Chr7: 95062361-95065361 |
| EID3 | Chr12: 103220178-103223178 | EID3, Chr12: 103220178-103223178 |
| C2orf39 | Chr2: 26476787-26479787 | C2orf39, Chr2: 26476787-26479787 |
| GSTM1 | Chr1: 110030464-110033464 | GSTM1, Chr1: 110030464-110033464 |
| C17orf46 | Chr17: 40693762-40696762 | C17orf46, Chr17: 40693762-40696762 |
| HEYL | Chr1: 39876435-39879435 | HEYL, Chr1: 39876435-39879435 |
| BAALC | Chr8: 104220596-104223596 | BAALC, Chr8: 104220596-104223596 |
| SERP2 | Chr13: 43844477-43847477 | SERP2, Chr13: 43844477-43847477 |
| AOX1 | Chr2: 201157475-201160475 | AOX1, Chr2: 201157475-201160475 |
| ADHFE1 | Chr8: 67505771-67508771 | ADHFE1, Chr8: 67505771-67508771 |
| SCTR | Chr2: 119996998-119999998 | SCTR, Chr2: 119996998-119999998 |
| PPP1R3C | Chr10: 93381338-93384338 | PPP1R3C, Chr10: 93381338-93384338 |
| GLP1R | Chr6: 39123034-39126034 | GLP1R, Chr6: 39123034-39126034 |
| C20orf85 | Chr20: 56157888-56160888 | C20orf85, Chr20: 56157888-56160888 |
| HIF3A | Chr19: 51490644-51493644 | HIF3A, Chr19: 51490644-51493644 |
| FAIM2 | Chr12: 48582487-48585487 | FAIM2, Chr12: 48582487-48585487 |
| G0S2 | Chr1: 207913792-207916792 | G0S2, Chr1: 207913792-207916792 |
| SLC22A16 | Chr6: 110903037-110906037 | SLC22A16, Chr6: 110903037-110906037 |
| MAPK15 | Chr8: 144868994-144871994 | MAPK15, Chr8: 144868994-144871994 |
| CAND2 | Chr3: 12811670-12814670 | CAND2, Chr3: 12811670-12814670 |
| COMP | Chr19: 18761614-18764614 | COMP, Chr19: 18761614-18764614 |
| PAX1 | Chr20: 21632796-21635796 | PAX1, Chr20: 21632796-21635796 |
| NEUROG3 | Chr10: 71001628-71004628 | NEUROG3, Chr10: 71001628-71004628 |
| C1QL2 | Chr2: 119631441-119634441 | C1QL2, Chr2: 119631441-119634441 |
| SLITRK4 | Chr23: 142549185-142552185 | SLITRK4, Chr23: 142549185-142552185 |
| LOXL2 | Chr8: 23316167-23319167 | LOXL2, Chr8: 23316167-23319167 |
| ST6GALNAC5 | Chr1: 77104273-77107273 | ST6GALNAC5, Chr1: 77104273-77107273 |
| CHST7 | Chr23: 46316635-46319635 | CHST7, Chr23: 46316635-46319635 |
| NKX2-4 | Chr20: 21324547-21327547 | NKX2-4, Chr20: 21324547-21327547 |
| FOSL1 | Chr11: 65423073-65426073 | FOSL1, Chr11: 65423073-65426073 |
| WNT7A | Chr3: 13895119-13898119 | WNT7A, Chr3: 13895119-13898119 |
| DPF3 | Chr14: 72429062-72432062 | DPF3, Chr14: 72429062-72432062 |
| EPHB1 | Chr3: 135995449-135998449 | EPHB1, Chr3: 135995449-135998449 |
| GSX2 | Chr4: 54659504-54662504 | GSX2, Chr4: 54659504-54662504 |
| HSPA6 | Chr1: 159759159-159762159 | HSPA6, Chr1: 159759159-159762159 |
| BMP4 | Chr14: 53491804-53494804 | BMP4, Chr14: 53491804-53494804 |
| KCNA6 | Chr12: 4787102-4790102 | KCNA6, Chr12: 4787102-4790102 |
| NDRG2 | Chr14: 20562275-20565275 | NDRG2, Chr14: 20562275-20565275 |
| EDIL3 | Chr5: 83714867-83717867 | EDIL3, Chr5: 83714867-83717867 |
| PLCXD3 | Chr5: 41544987-41547987 | PLCXD3, Chr5: 41544987-41547987 |
| VAMP5 | Chr2: 85663541-85666541 | VAMP5, Chr2: 85663541-85666541 |
| SLC6A17 | Chr1: 110493154-110496154 | SLC6A17, Chr1: 110493154-110496154 |
| FBXL10 | Chr12: 120501247-120504247 | FBXL10, Chr12: 120501247-120504247 |
| MEI1 | Chr22: 40423963-40426963 | MEI1, Chr22: 40423963-40426963 |
| C6orf150 | Chr6: 74217264-74220264 | C6orf150, Chr6: 74217264-74220264 |
| FGF13 | Chr23: 137619993-137622993 | FGF13, Chr23: 137619993-137622993 |
| ARID5A | Chr2: 96564690-96567690 | ARID5A, Chr2: 96564690-96567690 |
| GPC5 | Chr13: 90847387-90850387 | GPC5, Chr13: 90847387-90850387 |
| FLJ45422 | Chr6: 30333852-30336852 | FLJ45422, Chr6: 30333852-30336852 |
| AKR1B1 | Chr7: 133792928-133795928 | AKR1B1, Chr7: 133792928-133795928 |
| NFE2L3 | Chr7: 26156884-26159884 | NFE2L3, Chr7: 26156884-26159884 |
| PCSK9 | Chr1: 55276307-55279307 | PCSK9, Chr1: 55276307-55279307 |
| NR0B1 | Chr23: 30235916-30238916 | NR0B1, Chr23: 30235916-30238916 |
| MATK | Chr19: 3735915-3738915 | MATK, Chr19: 3735915-3738915 |
| AMMECR1 | Chr23: 109446536-109449536 | AMMECR1, Chr23: 109446536-109449536 |
| IFFO | Chr12: 6533990-6536990 | IFFO, Chr12: 6533990-6536990 |
| CHCHD6 | Chr3: 127904307-127907307 | CHCHD6, Chr3: 127904307-127907307 |
| RAB23 | Chr6: 57193537-57196537 | RAB23, Chr6: 57193537-57196537 |
| NOPE | Chr15: 63500963-63503963 | NOPE, Chr15: 63500963-63503963 |
| VAT1 | Chr17: 38426485-38429485 | VAT1, Chr17: 38426485-38429485 |
| CCDC3 | Chr10: 13082210-13085210 | CCDC3, Chr10: 13082210-13085210 |
| PLK3 | Chr1: 45037122-45040122 | PLK3, Chr1: 45037122-45040122 |
| CIDEB | Chr14: 23848916-23851916 | CIDEB, Chr14: 23848916-23851916 |
| CARTPT | Chr5: 71049249-71052249 | CARTPT, Chr5: 71049249-71052249 |
| NHLRC1 | Chr6: 18229330-18232330 | NHLRC1, Chr6: 18229330-18232330 |
| CENTG1 | Chr12: 56416796-56419796 | CENTG1, Chr12: 56416796-56419796 |
| RALYL | Chr8: 85256507-85259507 | RALYL, Chr8: 85256507-85259507 |
| CYP27A1 | Chr2: 219353215-219356215 | CYP27A1, Chr2: 219353215-219356215 |
| HMBOX1 | Chr8: 28802643-28805643 | HMBOX1, Chr8: 28802643-28805643 |
| C3orf31 | Chr3: 11861852-11864852 | C3orf31, Chr3: 11861852-11864852 |
| PPAPDC2 | Chr9: 4650797-4653797 | PPAPDC2, Chr9: 4650797-4653797 |
| RAB36 | Chr22: 21816012-21819012 | RAB36, Chr22: 21816012-21819012 |
| PFN3 | Chr5: 176758743-176761743 | PFN3, Chr5: 176758743-176761743 |
| C19orf51 | Chr19: 60368330-60371330 | C19orf51, Chr19: 60368330-60371330 |
| LOC440093 | Chr12: 31834942-31837942 | LOC440093, Chr12: 31834942-31837942 |
| ZNF577 | Chr19: 57081509-57084509 | ZNF577, Chr19: 57081509-57084509 |
| HEXA | Chr15: 70454074-70457074 | HEXA, Chr15: 70454074-70457074 |
| C1orf38 | Chr1: 28070141-28073141 | C1orf38, Chr1: 28070141-28073141 |
| C14orf39 | Chr14: 60021017-60024017 | C14orf39, Chr14: 60021017-60024017 |
| DEPDC7 | Chr11: 32992485-32995485 | DEPDC7, Chr11: 32992485-32995485 |
| SLC5A7 | Chr2: 107967926-107970926 | SLC5A7, Chr2: 107967926-107970926 |
| RASSF5 | Chr1: 204746001-204749001 | RASSF5, Chr1: 204746001-204749001 |
| EPSTI1 | Chr13: 42462877-42465877 | EPSTI1, Chr13: 42462877-42465877 |
| TAF6L | Chr11: 62293950-62296950 | TAF6L, Chr11: 62293950-62296950 |
| CXCL1 | Chr4: 74952472-74955472 | CXCL1, Chr4: 74952472-74955472 |
| ZSWIM2 | Chr2: 187420642-187423642 | ZSWIM2, Chr2: 187420642-187423642 |
| NKAPL | Chr6: 28333576-28336576 | NKAPL, Chr6: 28333576-28336576 |
| THBS4 | Chr5: 79365246-79368246 | THBS4, Chr5: 79365246-79368246 |
| ITGA7 | Chr12: 54386394-54389394 | ITGA7, Chr12: 54386394-54389394 |
| TTBK1 | Chr6: 43317699-43320699 | TTBK1, Chr6: 43317699-43320699 |
| GSTM4 | Chr1: 109998725-110001725 | GSTM4, Chr1: 109998725-110001725 |
| GLG1 | Chr16: 73197018-73200018 | GLG1, Chr16: 73197018-73200018 |
| ADAM32 | Chr8: 39082706-39085706 | ADAM32, Chr8: 39082706-39085706 |
| IRF6 | Chr1: 208044602-208047602 | IRF6, Chr1: 208044602-208047602 |
| DIRAS2 | Chr9: 92443428-92446428 | DIRAS2, Chr9: 92443428-92446428 |
| PSKH2 | Chr8: 87149467-87152467 | PSKH2, Chr8: 87149467-87152467 |
| SERPINB1 | Chr6: 2785580-2788580 | SERPINB1, Chr6: 2785580-2788580 |
| FAM47C | Chr23: 36934890-36937890 | FAM47C, Chr23: 36934890-36937890 |
| SPAG16 | Chr2: 213855860-213858860 | SPAG16, Chr2: 213855860-213858860 |
| CXCL2 | Chr4: 75182361-75185361 | CXCL2, Chr4: 75182361-75185361 |
| SHOX2 | Chr3: 159302020-159305020 | SHOX2, Chr3: 159302020-159305020 |
| COG2 | Chr1: 228843380-228846380 | COG2, Chr1: 228843380-228846380 |
| LOC285636 | Chr5: 41938726-41941726 | LOC285636, Chr5: 41938726-41941726 |
| GTF3A | Chr13: 26895180-26898180 | GTF3A, Chr13: 26895180-26898180 |
| CCDC37 | Chr3: 127594971-127597971 | CCDC37, Chr3: 127594971-127597971 |
| FAM163A | Chr1: 177977420-177980420 | FAM163A, Chr1: 177977420-177980420 |
| FOXD4 | Chr9: 106917-109917 | FOXD4, Chr9: 106917-109917 |
| GABRB3 | Chr15: 24568520-24571520 | GABRB3, Chr15: 24568520-24571520 |
| NTNG1 | Chr1: 107482651-107485651 | NTNG1, Chr1: 107482651-107485651 |
| SLC12A5 | Chr20: 44089744-44092744 | SLC12A5, Chr20: 44089744-44092744 |
| PRDM13 | Chr6: 100159870-100162870 | PRDM13, Chr6: 100159870-100162870 |
| ST8SIA3 | Chr18: 53169218-53172218 | ST8SIA3, Chr18: 53169218-53172218 |
| AFAP1L2 | Chr10: 116153005-116156005 | AFAP1L2, Chr10: 116153005-116156005 |
| DZIP1 | Chr13: 95093458-95096458 | DZIP1, Chr13: 95093458-95096458 |
| STMN1 | Chr1: 26104080-26107080 | STMN1, Chr1: 26104080-26107080 |
| ZNF154 | Chr19: 62910891-62913891 | ZNF154, Chr19: 62910891-62913891 |
| EGLN3 | Chr14: 33488535-33491535 | EGLN3, Chr14: 33488535-33491535 |
| TBPL2 | Chr14: 54975516-54978516 | TBPL2, Chr14: 54975516-54978516 |
| PDE4C | Chr19: 18196748-18199748 | PDE4C, Chr19: 18196748-18199748 |
| PTGER3 | Chr1: 71284579-71287579 | PTGER3, Chr1: 71284579-71287579 |
| APLN | Chr23: 128615095-128618095 | APLN, Chr23: 128615095-128618095 |
| MRPL53 | Chr2: 74551935-74554935 | MRPL53, Chr2: 74551935-74554935 |
| SEZ6 | Chr17: 24355707-24358707 | SEZ6, Chr17: 24355707-24358707 |
| APBB1 | Chr11: 6395720-6398720 | APBB1, Chr11: 6395720-6398720 |
| CTGF | Chr6: 132312711-132315711 | CTGF, Chr6: 132312711-132315711 |
| TTC22 | Chr1: 55038029-55041029 | TTC22, Chr1: 55038029-55041029 |
| DES | Chr2: 219989842-219992842 | DES, Chr2: 219989842-219992842 |
| WDR41 | Chr5: 76822588-76825588 | WDR41, Chr5: 76822588-76825588 |
| DPYS | Chr8: 105546953-105549953 | DPYS, Chr8: 105546953-105549953 |
| LOC342897 | Chr19: 44377943-44380943 | LOC342897, Chr19: 44377943-44380943 |
| C13orf33 | Chr13: 30376811-30379811 | C13orf33, Chr13: 30376811-30379811 |
| PYCARD | Chr16: 31120252-31123252 | PYCARD, Chr16: 31120252-31123252 |
| NEU1 | Chr6: 31937188-31940188 | NEU1, Chr6: 31937188-31940188 |
| HRASLS5 | Chr11: 63013742-63016742 | HRASLS5, Chr11: 63013742-63016742 |
| IPO4 | Chr14: 23726464-23729464 | IPO4, Chr14: 23726464-23729464 |
| GDA | Chr9: 73952612-73955612 | GDA, Chr9: 73952612-73955612 |
| SEC31B | Chr10: 102268085-102271085 | SEC31B, Chr10: 102268085-102271085 |
| NMNAT3 | Chr3: 140878030-140881030 | NMNAT3, Chr3: 140878030-140881030 |
| C3orf39 | Chr3: 43121069-43124069 | C3orf39, Chr3: 43121069-43124069 |
| VWA2 | Chr10: 115987507-115990507 | VWA2, Chr10: 115987507-115990507 |
| PIK3AP1 | Chr10: 98468769-98471769 | PIK3AP1, Chr10: 98468769-98471769 |
| DSG2 | Chr18: 27330524-27333524 | DSG2, Chr18: 27330524-27333524 |
| GPX3 | Chr5: 150378691-150381691 | GPX3, Chr5: 150378691-150381691 |
| CTSC | Chr11: 87709089-87712089 | CTSC, Chr11: 87709089-87712089 |
| FAM18A | Chr16: 10818622-10821622 | FAM18A, Chr16: 10818622-10821622 |
| LYPD5 | Chr19: 49015148-49018148 | LYPD5, Chr19: 49015148-49018148 |
| ANXA6 | Chr5: 150516060-150519060 | ANXA6, Chr5: 150516060-150519060 |
| MTO1 | Chr6: 74226674-74229674 | MTO1, Chr6: 74226674-74229674 |
| C10orf59 | Chr10: 90331450-90334450 | C10orf59, Chr10: 90331450-90334450 |
| C10orf59 | Chr10: 90331488-90334488 | C10orf59, Chr10: 90331488-90334488 |
| SYCN | Chr19: 44385246-44388246 | SYCN, Chr19: 44385246-44388246 |
| RHCG | Chr15: 87839303-87842303 | RHCG, Chr15: 87839303-87842303 |
| TNFRSF10C | Chr8: 23014878-23017878 | TNFRSF10C, Chr8: 23014878-23017878 |
| GPR75 | Chr2: 53939130-53942130 | GPR75, Chr2: 53939130-53942130 |
| DLEC1 | Chr3: 38054199-38057199 | DLEC1, Chr3: 38054199-38057199 |
| HAPLN3 | Chr15: 87238274-87241274 | HAPLN3, Chr15: 87238274-87241274 |
| DNASE2 | Chr19: 12851835-12854835 | DNASE2, Chr19: 12851835-12854835 |
| COPZ2 | Chr17: 43468651-43471651 | COPZ2, Chr17: 43468651-43471651 |
| RHOV | Chr15: 38952231-38955231 | RHOV, Chr15: 38952231-38955231 |
| PARP15 | Chr3: 123777638-123780638 | PARP15, Chr3: 123777638-123780638 |
| HIST1H2AG | Chr6: 27207299-27210299 | HIST1H2AG, Chr6: 27207299-27210299 |
| NOX4 | Chr11: 88862801-88865801 | NOX4, Chr11: 88862801-88865801 |
| HIST1H3E | Chr6: 26331861-26334861 | HIST1H3E, Chr6: 26331861-26334861 |
| PAPOLB | Chr7: 4866651-4869651 | PAPOLB, Chr7: 4866651-4869651 |
| ESR2 | Chr14: 63829381-63832381 | ESR2, Chr14: 63829381-63832381 |
| ME1 | Chr6: 84195998-84198998 | ME1, Chr6: 84195998-84198998 |
| CCKBR | Chr11: 6236041-6239041 | CCKBR, Chr11: 6236041-6239041 |
| C9orf68 | Chr9: 4654964-4657964 | C9orf68, Chr9: 4654964-4657964 |
| ZNF334 | Chr20: 44574101-44577101 | ZNF334, Chr20: 44574101-44577101 |
| SLC6A7 | Chr5: 149548212-149551212 | SLC6A7, Chr5: 149548212-149551212 |
| SALL4 | Chr20: 49850955-49853955 | SALL4, Chr20: 49850955-49853955 |
| KCNA1 | Chr12: 4887833-4890833 | KCNA1, Chr12: 4887833-4890833 |
| HOPX | Chr4: 57215908-57218908 | HOPX, Chr4: 57215908-57218908 |
| NEFM | Chr8: 24825678-24828678 | NEFM, Chr8: 24825678-24828678 |
| MOBKL2B | Chr9: 27518350-27521350 | MOBKL2B, Chr9: 27518350-27521350 |
| GPR143 | Chr23: 9692417-9695417 | GPR143, Chr23: 9692417-9695417 |
| AMN | Chr14: 102457245-102460245 | AMN, Chr14: 102457245-102460245 |
| NKX2-5 | Chr5: 172593368-172596368 | NKX2-5, Chr5: 172593368-172596368 |
| DAB1 | Chr1: 58487299-58490299 | DAB1, Chr1: 58487299-58490299 |
| SLC22A17 | Chr14: 22890420-22893420 | SLC22A17, Chr14: 22890420-22893420 |
| LAYN | Chr11: 110914942-110917942 | LAYN, Chr11: 110914942-110917942 |
| NGB | Chr14: 76805908-76808908 | NGB, Chr14: 76805908-76808908 |
| NES | Chr1: 154912313-154915313 | NES, Chr1: 154912313-154915313 |
| LOC130576 | Chr2: 149601726-149604726 | LOC130576, Chr2: 149601726-149604726 |
| ZNF662 | Chr3: 42920905-42923905 | ZNF662, Chr3: 42920905-42923905 |
| SFRP2 | Chr4: 154928178-154931178 | SFRP2, Chr4: 154928178-154931178 |
| FOXD4L1 | Chr2: 113971630-113974630 | FOXD4L1, Chr2: 113971630-113974630 |
| DBX2 | Chr12: 43729649-43732649 | DBX2, Chr12: 43729649-43732649 |
| SLC5A8 | Chr12: 100126647-100129647 | SLC5A8, Chr12: 100126647-100129647 |
| PDE4DIP | Chr1: 143785936-143788936 | PDE4DIP, Chr1: 143785936-143788936 |
| CRYGD | Chr2: 208696058-208699058 | CRYGD, Chr2: 208696058-208699058 |
| HIST3H2BB | Chr1: 226710930-226713930 | HIST3H2BB, Chr1: 226710930-226713930 |
| KCNE3 | Chr11: 73854748-73857748 | KCNE3, Chr11: 73854748-73857748 |
| LRCH2 | Chr23: 114373391-114376391 | LRCH2, Chr23: 114373391-114376391 |
| PTGIS | Chr20: 47616614-47619614 | PTGIS, Chr20: 47616614-47619614 |
| RASSF2 | Chr20: 4750791-4753791 | RASSF2, Chr20: 4750791-4753791 |
| ZNF671 | Chr19: 62929307-62932307 | ZNF671, Chr19: 62929307-62932307 |
| SYCP2L | Chr6: 10993549-10996549 | SYCP2L, Chr6: 10993549-10996549 |
| CORIN | Chr4: 47533316-47536316 | CORIN, Chr4: 47533316-47536316 |
| SLC13A3 | Chr20: 44712005-44715005 | SLC13A3, Chr20: 44712005-44715005 |
| VMO1 | Chr17: 4634967-4637967 | VMO1, Chr17: 4634967-4637967 |
| BCOR | Chr23: 39920026-39923026 | BCOR, Chr23: 39920026-39923026 |
| LYPD1 | Chr2: 133143451-133146451 | LYPD1, Chr2: 133143451-133146451 |
| HOXB8 | Chr17: 44045800-44048800 | HOXB8, Chr17: 44045800-44048800 |
| KCNK17 | Chr6: 39388714-39391714 | KCNK17, Chr6: 39388714-39391714 |
| N4BP2L1 | Chr13: 31898815-31901815 | N4BP2L1, Chr13: 31898815-31901815 |
| KCNK4 | Chr11: 63813868-63816868 | KCNK4, Chr11: 63813868-63816868 |
| RNF39 | Chr6: 30150107-30153107 | RNF39, Chr6: 30150107-30153107 |
| LVRN | Chr5: 115324549-115327549 | LVRN, Chr5: 115324549-115327549 |
| GNA14 | Chr9: 79451543-79454543 | GNA14, Chr9: 79451543-79454543 |
| MALL | Chr2: 110229437-110232437 | MALL, Chr2: 110229437-110232437 |
| SLC7A14 | Chr3: 171785057-171788057 | SLC7A14, Chr3: 171785057-171788057 |
| PCDHGA9 | Chr5: 140761203-140764203 | PCDHGA9, Chr5: 140761203-140764203 |
| ARL9 | Chr4: 57064631-57067631 | ARL9, Chr4: 57064631-57067631 |
| ZIC1 | Chr3: 148608370-148611370 | ZIC1, Chr3: 148608370-148611370 |
| TMTC1 | Chr12: 29827459-29830459 | TMTC1, Chr12: 29827459-29830459 |
| WNT16 | Chr7: 120754825-120757825 | WNT16, Chr7: 120754825-120757825 |
| NUAK1 | Chr12: 105056441-105059441 | NUAK1, Chr12: 105056441-105059441 |
| GALC | Chr14: 87528160-87531160 | GALC, Chr14: 87528160-87531160 |
| PCDHGA12 | Chr5: 140788841-140791841 | PCDHGA12, Chr5: 140788841-140791841 |
| LECT1 | Chr13: 52210448-52213448 | LECT1, Chr13: 52210448-52213448 |
| POU3F4 | Chr23: 82648440-82651440 | POU3F4, Chr23: 82648440-82651440 |
| TMEM220 | Chr17: 10572871-10575871 | TMEM220, Chr17: 10572871-10575871 |
| CNN1 | Chr19: 11509078-11512078 | CNN1, Chr19: 11509078-11512078 |
| KLHL1 | Chr13: 69578960-69581960 | KLHL1, Chr13: 69578960-69581960 |
| CD44 | Chr11: 35115492-35118492 | CD44, Chr11: 35115492-35118492 |
| BTN1A1 | Chr6: 26607973-26610973 | BTN1A1, Chr6: 26607973-26610973 |
| BTBD11 | Chr12: 106497263-106500263 | BTBD11, Chr12: 106497263-106500263 |
| FOLH1 | Chr11: 49185298-49188298 | FOLH1, Chr11: 49185298-49188298 |
| NTF3 | Chr12: 5472058-5475058 | NTF3, Chr12: 5472058-5475058 |
| GRIA4 | Chr11: 104984509-104987509 | GRIA4, Chr11: 104984509-104987509 |
| LYNX1 | Chr8: 143855141-143858141 | LYNX1, Chr8: 143855141-143858141 |
| LYNX1 | Chr8: 143855142-143858142 | LYNX1, Chr8: 143855142-143858142 |
| ADRB3 | Chr8: 37941841-37944841 | ADRB3, Chr8: 37941841-37944841 |
| FAM148B | Chr15: 60243274-60246274 | FAM148B, Chr15: 60243274-60246274 |
| STMN1 | Chr1: 26104455-26107455 | STMN1, Chr1: 26104455-26107455 |
| CD14 | Chr5: 139991694-139994694 | CD14, Chr5: 139991694-139994694 |
| CYP24A1 | Chr20: 52222423-52225423 | CYP24A1, Chr20: 52222423-52225423 |
| IL1RL2 | Chr2: 102168364-102171364 | IL1RL2, Chr2: 102168364-102171364 |
| C3orf44 | Chr3: 151902932-151905932 | C3orf44, Chr3: 151902932-151905932 |
| TMEM30B | Chr14: 60816783-60819783 | TMEM30B, Chr14: 60816783-60819783 |
| IGF2BP3 | Chr7: 23475020-23478020 | IGF2BP3, Chr7: 23475020-23478020 |
| NBL1 | Chr1: 19840812-19843812 | NBL1, Chr1: 19840812-19843812 |
| ZIC5 | Chr13: 99420679-99423679 | ZIC5, Chr13: 99420679-99423679 |
| FLJ45803 | Chr11: 110674249-110677249 | FLJ45803, Chr11: 110674249-110677249 |
| LOC399947 | Chr11: 108796584-108799584 | LOC399947, Chr11: 108796584-108799584 |
| VSX2 | Chr14: 73774427-73777427 | VSX2, Chr14: 73774427-73777427 |
| SPOCK2 | Chr10: 73517273-73520273 | SPOCK2, Chr10: 73517273-73520273 |
| C9orf125 | Chr9: 103287796-103290796 | C9orf125, Chr9: 103287796-103290796 |
| LHX9 | Chr1: 196151639-196154639 | LHX9, Chr1: 196151639-196154639 |
| DDN | Chr12: 47677855-47680855 | DDN, Chr12: 47677855-47680855 |
| MAB21L1 | Chr13: 34947288-34950288 | MAB21L1, Chr13: 34947288-34950288 |
| VASH2 | Chr1: 211189099-211192099 | VASH2, Chr1: 211189099-211192099 |
| CD14 | Chr5: 139991939-139994939 | CD14, Chr5: 139991939-139994939 |
| MAPK8IP2 | Chr22: 49386927-49389927 | MAPK8IP2, Chr22: 49386927-49389927 |
| S100A2 | Chr1: 151803430-151806430 | S100A2, Chr1: 151803430-151806430 |
| OTX2 | Chr14: 56345437-56348437 | OTX2, Chr14: 56345437-56348437 |
| KLK10 | Chr19: 56213266-56216266 | KLK10, Chr19: 56213266-56216266 |
| WFDC2 | Chr20: 43530307-43533307 | WFDC2, Chr20: 43530307-43533307 |
| LRRC4C | Chr11: 40270740-40273740 | LRRC4C, Chr11: 40270740-40273740 |
| L1TD1 | Chr1: 62431583-62434583 | L1TD1, Chr1: 62431583-62434583 |
| GPR88 | Chr1: 100774815-100777815 | GPR88, Chr1: 100774815-100777815 |
| OLIG2 | Chr21: 33318608-33321608 | OLIG2, Chr21: 33318608-33321608 |
| OSAP | Chr4: 140419442-140422442 | OSAP, Chr4: 140419442-140422442 |
| LYPD1 | Chr2: 133144040-133147040 | LYPD1, Chr2: 133144040-133147040 |
| MAFF | Chr22: 36937987-36940987 | MAFF, Chr22: 36937987-36940987 |
| PCDHGA8 | Chr5: 140750166-140753166 | PCDHGA8, Chr5: 140750166-140753166 |
| HNF1B | Chr17: 33177709-33180709 | HNF1B, Chr17: 33177709-33180709 |
| GPNMB | Chr7: 23251340-23254340 | GPNMB, Chr7: 23251340-23254340 |
| DNAJC6 | Chr1: 65501517-65504517 | DNAJC6, Chr1: 65501517-65504517 |
| CD79A | Chr19: 47071529-47074529 | CD79A, Chr19: 47071529-47074529 |
| FAM19A2 | Chr12: 60871318-60874318 | FAM19A2, Chr12: 60871318-60874318 |
| ACTA1 | Chr1: 227634966-227637966 | ACTA1, Chr1: 227634966-227637966 |
| GPR149 | Chr3: 155628698-155631698 | GPR149, Chr3: 155628698-155631698 |
| NKX2-2 | Chr20: 21441164-21444164 | NKX2-2, Chr20: 21441164-21444164 |
| KLK10 | Chr19: 56213594-56216594 | KLK10, Chr19: 56213594-56216594 |
| KLK10 | Chr19: 56213743-56216743 | KLK10, Chr19: 56213743-56216743 |
| LTK | Chr15: 39592291-39595291 | LTK, Chr15: 39592291-39595291 |
| GABRB1 | Chr4: 46726551-46729551 | GABRB1, Chr4: 46726551-46729551 |
| C20orf103 | Chr20: 9441770-9444770 | C20orf103, Chr20: 9441770-9444770 |
| PCDHA2 | Chr5: 140153127-140156127 | PCDHA2, Chr5: 140153127-140156127 |
| HIST1H4L | Chr6: 27947768-27950768 | HIST1H4L, Chr6: 27947768-27950768 |
| DDAH2 | Chr6: 31804518-31807518 | DDAH2, Chr6: 31804518-31807518 |
| ADD3 | Chr10: 111754215-111757215 | ADD3, Chr10: 111754215-111757215 |
| HPSE2 | Chr10: 100984109-100987109 | HPSE2, Chr10: 100984109-100987109 |
| LGALS1 | Chr22: 36400058-36403058 | LGALS1, Chr22: 36400058-36403058 |
| LRRN4CL | Chr11: 62212276-62215276 | LRRN4CL, Chr11: 62212276-62215276 |
| ACP1 | Chr2: 253395-256395 | ACP1, Chr2: 253395-256395 |
| TD1 | Chr10: 102879393-102882393 | TD1, Chr10: 102879393-102882393 |
| ACP1 | Chr2: 253371-256371 | ACP1, Chr2: 253371-256371 |
| PIGW | Chr17: 31964015-31967015 | PIGW, Chr17: 31964015-31967015 |
| HMGA1 | Chr6: 34311873-34314873 | HMGA1, Chr6: 34311873-34314873 |
| SMAD3 | Chr15: 65143748-65146748 | SMAD3, Chr15: 65143748-65146748 |
| USH1G | Chr17: 70429446-70432446 | USH1G, Chr17: 70429446-70432446 |
| CHST8 | Chr19: 38803200-38806200 | CHST8, Chr19: 38803200-38806200 |
| CHST8 | Chr19: 38803212-38806212 | CHST8, Chr19: 38803212-38806212 |
| SH3BP4 | Chr2: 235523866-235526866 | SH3BP4, Chr2: 235523866-235526866 |
| RPL8 | Chr8: 145987033-145990033 | RPL8, Chr8: 145987033-145990033 |
| GNB1 | Chr1: 1810855-1813855 | GNB1, Chr1: 1810855-1813855 |
| TPPP | Chr5: 745010-748010 | TPPP, Chr5: 745010-748010 |
| RPL8 | Chr8: 145987109-145990109 | RPL8, Chr8: 145987109-145990109 |
| GDI1 | Chr23: 153316952-153319952 | GDI1, Chr23: 153316952-153319952 |
| FAM160A1 | Chr4: 152548347-152551347 | FAM160A1, Chr4: 152548347-152551347 |
| ATP5I | Chr4: 656622-659622 | ATP5I, Chr4: 656622-659622 |
| NTNG2 | Chr9: 134025654-134028654 | NTNG2, Chr9: 134025654-134028654 |
| PRKY | Chr24: 7200513-7203513 | PRKY, Chr24: 7200513-7203513 |
| FAM89A | Chr1: 229241108-229244108 | FAM89A, Chr1: 229241108-229244108 |
| SURF1 | Chr9: 135211682-135214682 | SURF1, Chr9: 135211682-135214682 |
| DCUN1D2 | Chr13: 113191524-113194524 | DCUN1D2, Chr13: 113191524-113194524 |
| RPL12 | Chr9: 129252005-129255005 | RPL12, Chr9: 129252005-129255005 |
| CHST12 | Chr7: 2408284-2411284 | CHST12, Chr7: 2408284-2411284 |
| C12orf23 | Chr12: 105872334-105875334 | C12orf23, Chr12: 105872334-105875334 |
| ALG12 | Chr22: 48696610-48699610 | ALG12, Chr22: 48696610-48699610 |
| CCHCR1 | Chr6: 31232494-31235494 | CCHCR1, Chr6: 31232494-31235494 |
| SNAI1 | Chr20: 48031433-48034433 | SNAI1, Chr20: 48031433-48034433 |
| H6PD | Chr1: 9215949-9218949 | H6PD, Chr1: 9215949-9218949 |
| EPHB4 | Chr7: 100261579-100264579 | EPHB4, Chr7: 100261579-100264579 |
| POLD1 | Chr19: 55577904-55580904 | POLD1, Chr19: 55577904-55580904 |
| FLJ10357 | Chr14: 20606866-20609866 | FLJ10357, Chr14: 20606866-20609866 |
| FAM123C | Chr2: 131228833-131231833 | FAM123C, Chr2: 131228833-131231833 |
| BCAR1 | Chr16: 73841504-73844504 | BCAR1, Chr16: 73841504-73844504 |
| PODXL | Chr7: 130890416-130893416 | PODXL, Chr7: 130890416-130893416 |
| HIGD1A | Chr3: 42819531-42822531 | HIGD1A, Chr3: 42819531-42822531 |
| DCBLD1 | Chr6: 117909012-117912012 | DCBLD1, Chr6: 117909012-117912012 |
| HIGD1A | Chr3: 42819438-42822438 | HIGD1A, Chr3: 42819438-42822438 |
| LIMK1 | Chr7: 73134591-73137591 | LIMK1, Chr7: 73134591-73137591 |
| PPP2R2A | Chr8: 26203450-26206450 | PPP2R2A, Chr8: 26203450-26206450 |
| NFATC3 | Chr16: 66675375-66678375 | NFATC3, Chr16: 66675375-66678375 |
| ANKRD20A3 | Chr9: 43122040-43125040 | ANKRD20A3, Chr9: 43122040-43125040 |
| THRA | Chr17: 35471088-35474088 | THRA, Chr17: 35471088-35474088 |
| CARHSP1 | Chr16: 8868249-8871249 | CARHSP1, Chr16: 8868249-8871249 |
| FAM134C | Chr17: 38013428-38016428 | FAM134C, Chr17: 38013428-38016428 |
| RING1 | Chr6: 33282763-33285763 | RING1, Chr6: 33282763-33285763 |
| ADAMTS7 | Chr15: 76889328-76892328 | ADAMTS7, Chr15: 76889328-76892328 |
| HEBP2 | Chr6: 138765528-138768528 | HEBP2, Chr6: 138765528-138768528 |
| SPRY4 | Chr5: 141683304-141686304 | SPRY4, Chr5: 141683304-141686304 |
| MARK1 | Chr1: 218766690-218769690 | MARK1, Chr1: 218766690-218769690 |
| SLC12A4 | Chr16: 66558526-66561526 | SLC12A4, Chr16: 66558526-66561526 |
| LAMA5 | Chr20: 60374263-60377263 | LAMA5, Chr20: 60374263-60377263 |
| CRABP2 | Chr1: 154940499-154943499 | CRABP2, Chr1: 154940499-154943499 |
| ALX1 | Chr12: 84196666-84199666 | ALX1, Chr12: 84196666-84199666 |
| SIRT6 | Chr19: 4132096-4135096 | SIRT6, Chr19: 4132096-4135096 |
| SYT6 | Chr1: 114496495-114499495 | SYT6, Chr1: 114496495-114499495 |
| LARP1 | Chr5: 154113581-154116581 | LARP1, Chr5: 154113581-154116581 |
| HLF | Chr17: 50695819-50698819 | HLF, Chr17: 50695819-50698819 |
| EIF4G1 | Chr3: 185513549-185516549 | EIF4G1, Chr3: 185513549-185516549 |
| SUMO1 | Chr2: 202810067-202813067 | SUMO1, Chr2: 202810067-202813067 |
| CD58 | Chr1: 116913626-116916626 | CD58, Chr1: 116913626-116916626 |
| PPP1R12C | Chr19: 60319239-60322239 | PPP1R12C, Chr19: 60319239-60322239 |
| FBLIM1 | Chr1: 15956341-15959341 | FBLIM1, Chr1: 15956341-15959341 |
| AKAP10 | Chr17: 19820221-19823221 | AKAP10, Chr17: 19820221-19823221 |
| MFN2 | Chr1: 11961324-11964324 | MFN2, Chr1: 11961324-11964324 |
| SCAMP2 | Chr15: 72951223-72954223 | SCAMP2, Chr15: 72951223-72954223 |
| C16orf74 | Chr16: 84340690-84343690 | C16orf74, Chr16: 84340690-84343690 |
| ATP8A1 | Chr4: 42352379-42355379 | ATP8A1, Chr4: 42352379-42355379 |
| GEFT | Chr12: 56289984-56292984 | GEFT, Chr12: 56289984-56292984 |
| FLNA | Chr23: 153254700-153257700 | FLNA, Chr23: 153254700-153257700 |
| SHISA5 | Chr3: 48515165-48518165 | SHISA5, Chr3: 48515165-48518165 |
| SLC38A1 | Chr12: 44947975-44950975 | SLC38A1, Chr12: 44947975-44950975 |
| MGA | Chr15: 39738401-39741401 | MGA, Chr15: 39738401-39741401 |
| FBXO21 | Chr12: 116111183-116114183 | FBXO21, Chr12: 116111183-116114183 |
| TESC | Chr12: 116020134-116023134 | TESC, Chr12: 116020134-116023134 |
| C3orf25 | Chr3: 130628684-130631684 | C3orf25, Chr3: 130628684-130631684 |
| ZBED3 | Chr5: 76417286-76420286 | ZBED3, Chr5: 76417286-76420286 |
| KIF16B | Chr20: 16500578-16503578 | KIF16B, Chr20: 16500578-16503578 |
| NGF | Chr1: 115680880-115683880 | NGF, Chr1: 115680880-115683880 |
| ITIH5 | Chr10: 7747440-7750440 | ITIH5, Chr10: 7747440-7750440 |
| LOC100128378 | Chr3: 52072668-52075668 | LOC100128378, Chr3: 52072668-52075668 |
| FAM123C | Chr2: 131228193-131231193 | FAM123C, Chr2: 131228193-131231193 |
| TBX5 | Chr12: 113326772-113329772 | TBX5, Chr12: 113326772-113329772 |
| HSPB6 | Chr19: 40938270-40941270 | HSPB6, Chr19: 40938270-40941270 |
| CARHSP1 | Chr16: 8868864-8871864 | CARHSP1, Chr16: 8868864-8871864 |
| APBB1IP | Chr10: 26765771-26768771 | APBB1IP, Chr10: 26765771-26768771 |
| KIAA1683 | Chr19: 18244735-18247735 | KIAA1683, Chr19: 18244735-18247735 |
| FAM123C | Chr2: 131228046-131231046 | FAM123C, Chr2: 131228046-131231046 |
| CSMD2 | Chr1: 34402530-34405530 | CSMD2, Chr1: 34402530-34405530 |
| C18orf51 | Chr18: 70274659-70277659 | C18orf51, Chr18: 70274659-70277659 |
| CFD | Chr19: 809164-812164 | CFD, Chr19: 809164-812164 |
| SLC7A8 | Chr14: 22691950-22694950 | SLC7A8, Chr14: 22691950-22694950 |
| MAGIX | Chr23: 48904624-48907624 | MAGIX, Chr23: 48904624-48907624 |
| ADAMTS19 | Chr5: 128822501-128825501 | ADAMTS19, Chr5: 128822501-128825501 |
| LHX5 | Chr12: 112392760-112395760 | LHX5, Chr12: 112392760-112395760 |
| THY1 | Chr11: 118797564-118800564 | THY1, Chr11: 118797564-118800564 |
| TAC1 | Chr7: 97197706-97200706 | TAC1, Chr7: 97197706-97200706 |
| HIST1H4F | Chr6: 26347132-26350132 | HIST1H4F, Chr6: 26347132-26350132 |
| KRT8 | Chr12: 51583627-51586627 | KRT8, Chr12: 51583627-51586627 |
| NHLH2 | Chr1: 116183356-116186356 | NHLH2, Chr1: 116183356-116186356 |
| IL22RA1 | Chr1: 24340698-24343698 | IL22RA1, Chr1: 24340698-24343698 |
| C1QTNF1 | Chr17: 74540566-74543566 | C1QTNF1, Chr17: 74540566-74543566 |
| ETV2 | Chr19: 40822986-40825986 | ETV2, Chr19: 40822986-40825986 |
| PMP22 | Chr17: 15105114-15108114 | PMP22, Chr17: 15105114-15108114 |
| C1orf183 | Chr1: 112082045-112085045 | C1orf183, Chr1: 112082045-112085045 |
| CASKIN2 | Chr17: 71021722-71024722 | CASKIN2, Chr17: 71021722-71024722 |
| EEF1D | Chr8: 144749488-144752488 | EEF1D, Chr8: 144749488-144752488 |
| DBNDD2 | Chr20: 43467150-43470150 | DBNDD2, Chr20: 43467150-43470150 |
| KCNK5 | Chr6: 39303729-39306729 | KCNK5, Chr6: 39303729-39306729 |
| EVI5L | Chr19: 7799742-7802742 | EVI5L, Chr19: 7799742-7802742 |
| LRSAM1 | Chr9: 129252854-129255854 | LRSAM1, Chr9: 129252854-129255854 |
| CDX4 | Chr23: 72582314-72585314 | CDX4, Chr23: 72582314-72585314 |
| RARRES2 | Chr7: 149668139-149671139 | RARRES2, Chr7: 149668139-149671139 |
| LHX9 | Chr1: 196146757-196149757 | LHX9, Chr1: 196146757-196149757 |
| KLF12 | Chr13: 73604567-73607567 | KLF12, Chr13: 73604567-73607567 |
| FKBP2 | Chr11: 63764663-63767663 | FKBP2, Chr11: 63764663-63767663 |
| HES3 | Chr1: 6225348-6228348 | HES3, Chr1: 6225348-6228348 |
| IFITM3 | Chr11: 309414-312414 | IFITM3, Chr11: 309414-312414 |
| PRDM8 | Chr4: 81323947-81326947 | PRDM8, Chr4: 81323947-81326947 |
| ZNF236 | Chr18: 72663603-72666603 | ZNF236, Chr18: 72663603-72666603 |
| GPR101 | Chr23: 135939999-135942999 | GPR101, Chr23: 135939999-135942999 |
| UGT1A1 | Chr2: 234332157-234335157 | UGT1A1, Chr2: 234332157-234335157 |
| APBA3 | Chr19: 3711173-3714173 | APBA3, Chr19: 3711173-3714173 |
| TENC1 | Chr12: 51727499-51730499 | TENC1, Chr12: 51727499-51730499 |
| AMT | Chr3: 49433516-49436516 | AMT, Chr3: 49433516-49436516 |
| FTSJ1 | Chr23: 48219823-48222823 | FTSJ1, Chr23: 48219823-48222823 |
| DBNDD2 | Chr20: 43468828-43471828 | DBNDD2, Chr20: 43468828-43471828 |
| TRIM31 | Chr6: 30187346-30190346 | TRIM31, Chr6: 30187346-30190346 |
| C1orf94 | Chr1: 34403710-34406710 | C1orf94, Chr1: 34403710-34406710 |
| PCSK4 | Chr19: 1439907-1442907 | PCSK4, Chr19: 1439907-1442907 |
| SP100 | Chr2: 230987614-230990614 | SP100, Chr2: 230987614-230990614 |
| C10orf82 | Chr10: 118417971-118420971 | C10orf82, Chr10: 118417971-118420971 |
| OTOP2 | Chr17: 70430464-70433464 | OTOP2, Chr17: 70430464-70433464 |
| SLC25A35 | Chr17: 8137395-8140395 | SLC25A35, Chr17: 8137395-8140395 |
| SPATS1 | Chr6: 44416963-44419963 | SPATS1, Chr6: 44416963-44419963 |
| ARHGEF17 | Chr11: 72695816-72698816 | ARHGEF17, Chr11: 72695816-72698816 |
| NXNL2 | Chr9: 90338339-90341339 | NXNL2, Chr9: 90338339-90341339 |
| ZNF833 | Chr19: 11644307-11647307 | ZNF833, Chr19: 11644307-11647307 |
| TGFB1I1 | Chr16: 31390526-31393526 | TGFB1I1, Chr16: 31390526-31393526 |
| DBNDD2 | Chr20: 43466584-43469584 | DBNDD2, Chr20: 43466584-43469584 |
| PPP1R14B | Chr11: 63769489-63772489 | PPP1R14B, Chr11: 63769489-63772489 |
| MMP25 | Chr16: 3035182-3038182 | MMP25, Chr16: 3035182-3038182 |
| GCM2 | Chr6: 10988584-10991584 | GCM2, Chr6: 10988584-10991584 |
| HHEX | Chr10: 94438160-94441160 | HHEX, Chr10: 94438160-94441160 |
| PLEKHG2 | Chr19: 44594089-44597089 | PLEKHG2, Chr19: 44594089-44597089 |
| LOC129293 | Chr2: 84960263-84963263 | LOC129293, Chr2: 84960263-84963263 |
| ELP2 | Chr18: 31962384-31965384 | ELP2, Chr18: 31962384-31965384 |
| NAV2 | Chr11: 19689956-19692956 | NAV2, Chr11: 19689956-19692956 |
| KCNK10 | Chr14: 87857600-87860600 | KCNK10, Chr14: 87857600-87860600 |
| POU2F2 | Chr19: 47326970-47329970 | POU2F2, Chr19: 47326970-47329970 |
| HLX | Chr1: 219117865-219120865 | HLX, Chr1: 219117865-219120865 |
| LRRN1 | Chr3: 3814620-3817620 | LRRN1, Chr3: 3814620-3817620 |
| NKD1 | Chr16: 49138241-49141241 | NKD1, Chr16: 49138241-49141241 |
| TMCO3 | Chr13: 113191808-113194808 | TMCO3, Chr13: 113191808-113194808 |
| MYO19 | Chr17: 31963337-31966337 | MYO19, Chr17: 31963337-31966337 |
| MYO19 | Chr17: 31963338-31966338 | MYO19, Chr17: 31963338-31966338 |
| LRSAM1 | Chr9: 129252337-129255337 | LRSAM1, Chr9: 129252337-129255337 |
| B4GALNT4 | Chr11: 358303-361303 | B4GALNT4, Chr11: 358303-361303 |
| ZNF623 | Chr8: 144788015-144791015 | ZNF623, Chr8: 144788015-144791015 |
| CAPN1 | Chr11: 64704418-64707418 | CAPN1, Chr11: 64704418-64707418 |
| WNT4 | Chr1: 22340606-22343606 | WNT4, Chr1: 22340606-22343606 |
| BICD2 | Chr9: 94565404-94568404 | BICD2, Chr9: 94565404-94568404 |
| PHLDA1 | Chr12: 74710323-74713323 | PHLDA1, Chr12: 74710323-74713323 |
| LRSAM1 | Chr9: 129252112-129255112 | LRSAM1, Chr9: 129252112-129255112 |
| MDK | Chr11: 46358378-46361378 | MDK, Chr11: 46358378-46361378 |
| TM6SF1 | Chr15: 81565883-81568883 | TM6SF1, Chr15: 81565883-81568883 |
| RGS7BP | Chr5: 63836707-63839707 | RGS7BP, Chr5: 63836707-63839707 |
| LYPD6 | Chr2: 149893858-149896858 | LYPD6, Chr2: 149893858-149896858 |
| SH3PXD2A | Chr10: 105603654-105606654 | SH3PXD2A, Chr10: 105603654-105606654 |
| MDK | Chr11: 46358294-46361294 | MDK, Chr11: 46358294-46361294 |
| HS3ST1 | Chr4: 11038135-11041135 | HS3ST1, Chr4: 11038135-11041135 |
| CD9 | Chr12: 6178315-6181315 | CD9, Chr12: 6178315-6181315 |
| SSH1 | Chr12: 107773980-107776980 | SSH1, Chr12: 107773980-107776980 |
| CRELD2 | Chr22: 48696847-48699847 | CRELD2, Chr22: 48696847-48699847 |
| SNCAIP | Chr5: 121674218-121677218 | SNCAIP, Chr5: 121674218-121677218 |
| HBA2 | Chr16: 161345-164345 | HBA2, Chr16: 161345-164345 |
| CTTN | Chr11: 69920759-69923759 | CTTN, Chr11: 69920759-69923759 |
| CTTN | Chr11: 69920791-69923791 | CTTN, Chr11: 69920791-69923791 |
| ADCYAP1R1 | Chr7: 31057166-31060166 | ADCYAP1R1, Chr7: 31057166-31060166 |
| ARVCF | Chr22: 18382809-18385809 | ARVCF, Chr22: 18382809-18385809 |
| ITM2C | Chr2: 231436364-231439364 | ITM2C, Chr2: 231436364-231439364 |
| N4BP3 | Chr5: 177471661-177474661 | N4BP3, Chr5: 177471661-177474661 |
| GPRC5B | Chr16: 19802152-19805152 | GPRC5B, Chr16: 19802152-19805152 |
| ADAM22 | Chr7: 87400137-87403137 | ADAM22, Chr7: 87400137-87403137 |
| TCF19 | Chr6: 31232781-31235781 | TCF19, Chr6: 31232781-31235781 |
| RUSC2 | Chr9: 35478623-35481623 | RUSC2, Chr9: 35478623-35481623 |
| TUBG1 | Chr17: 38013719-38016719 | TUBG1, Chr17: 38013719-38016719 |
| SDF2L1 | Chr22: 20325041-20328041 | SDF2L1, Chr22: 20325041-20328041 |
| ECE1 | Chr1: 21543121-21546121 | ECE1, Chr1: 21543121-21546121 |
| NNT | Chr5: 43637485-43640485 | NNT, Chr5: 43637485-43640485 |
| ATP12A | Chr13: 24151194-24154194 | ATP12A, Chr13: 24151194-24154194 |
| GRP | Chr18: 55036879-55039879 | GRP, Chr18: 55036879-55039879 |
| KIAA0528 | Chr12: 22587219-22590219 | KIAA0528, Chr12: 22587219-22590219 |
| CLIP2 | Chr7: 73340240-73343240 | CLIP2, Chr7: 73340240-73343240 |
| ADA | Chr20: 42712290-42715290 | ADA, Chr20: 42712290-42715290 |
| ARHGDIG | Chr16: 269106-272106 | ARHGDIG, Chr16: 269106-272106 |
| LRRN2 | Chr1: 202919604-202922604 | LRRN2, Chr1: 202919604-202922604 |
| FMNL3 | Chr12: 48385964-48388964 | FMNL3, Chr12: 48385964-48388964 |
| MFSD2 | Chr1: 40191896-40194896 | MFSD2, Chr1: 40191896-40194896 |
| IGSF9 | Chr1: 158180510-158183510 | IGSF9, Chr1: 158180510-158183510 |
| PODN | Chr1: 53298972-53301972 | PODN, Chr1: 53298972-53301972 |
| MYLK | Chr3: 125084339-125087339 | MYLK, Chr3: 125084339-125087339 |
| TRPS1 | Chr8: 116748902-116751902 | TRPS1, Chr8: 116748902-116751902 |
| CAMK2B | Chr7: 44330249-44333249 | CAMK2B, Chr7: 44330249-44333249 |
| TENC1 | Chr12: 51725576-51728576 | TENC1, Chr12: 51725576-51728576 |
| OPRS1 | Chr9: 34626268-34629268 | OPRS1, Chr9: 34626268-34629268 |
| PSEN2 | Chr1: 225123395-225126395 | PSEN2, Chr1: 225123395-225126395 |
| MT3 | Chr16: 55179267-55182267 | MT3, Chr16: 55179267-55182267 |
| GPC3 | Chr23: 132945832-132948832 | GPC3, Chr23: 132945832-132948832 |
| LOC134121 | Chr5: 7902764-7905764 | LOC134121, Chr5: 7902764-7905764 |
| SERINC2 | Chr1: 31657049-31660049 | SERINC2, Chr1: 31657049-31660049 |
| ZC3H3 | Chr8: 144693263-144696263 | ZC3H3, Chr8: 144693263-144696263 |
| MYBBP1A | Chr17: 4403930-4406930 | MYBBP1A, Chr17: 4403930-4406930 |
| OLFML2A | Chr9: 126577757-126580757 | OLFML2A, Chr9: 126577757-126580757 |
| LRRN2 | Chr1: 202919720-202922720 | LRRN2, Chr1: 202919720-202922720 |
| TSHZ2 | Chr20: 51020783-51023783 | TSHZ2, Chr20: 51020783-51023783 |
| ORAI2 | Chr7: 101859500-101862500 | ORAI2, Chr7: 101859500-101862500 |
| MAPK13 | Chr6: 36204739-36207739 | MAPK13, Chr6: 36204739-36207739 |
| CITED4 | Chr1: 41099105-41102105 | CITED4, Chr1: 41099105-41102105 |
| NTN4 | Chr12: 94707167-94710167 | NTN4, Chr12: 94707167-94710167 |
| MAP4 | Chr3: 48104214-48107214 | MAP4, Chr3: 48104214-48107214 |
| MAP4 | Chr3: 48104215-48107215 | MAP4, Chr3: 48104215-48107215 |
| KHDRBS3 | Chr8: 136537397-136540397 | KHDRBS3, Chr8: 136537397-136540397 |
| C19orf66 | Chr19: 10056305-10059305 | C19orf66, Chr19: 10056305-10059305 |
| ADC | Chr1: 33317800-33320800 | ADC, Chr1: 33317800-33320800 |
| ULK1 | Chr12: 130943731-130946731 | ULK1, Chr12: 130943731-130946731 |
| TMEM145 | Chr19: 47507816-47510816 | TMEM145, Chr19: 47507816-47510816 |
| MMP28 | Chr17: 31145253-31148253 | MMP28, Chr17: 31145253-31148253 |
| LOC729085 | Chr3: 42994332-42997332 | LOC729085, Chr3: 42994332-42997332 |
| CHST9 | Chr18: 23017779-23020779 | CHST9, Chr18: 23017779-23020779 |
| TAGLN2 | Chr1: 158160408-158163408 | TAGLN2, Chr1: 158160408-158163408 |
| IGFBP1 | Chr7: 45892983-45895983 | IGFBP1, Chr7: 45892983-45895983 |
| FEV | Chr2: 219557123-219560123 | FEV, Chr2: 219557123-219560123 |
| OLFM1 | Chr9: 137105409-137108409 | OLFM1, Chr9: 137105409-137108409 |
| OLFM1 | Chr9: 137105423-137108423 | OLFM1, Chr9: 137105423-137108423 |
| MFGE8 | Chr15: 87256167-87259167 | MFGE8, Chr15: 87256167-87259167 |
| EPHA1 | Chr7: 142814607-142817607 | EPHA1, Chr7: 142814607-142817607 |
| NSUN5 | Chr7: 72359259-72362259 | NSUN5, Chr7: 72359259-72362259 |
| TNFSF11 | Chr13: 42044797-42047797 | TNFSF11, Chr13: 42044797-42047797 |
| RAP1GAP | Chr1: 21866886-21869886 | RAP1GAP, Chr1: 21866886-21869886 |
| CPT1B | Chr22: 49362244-49365244 | CPT1B, Chr22: 49362244-49365244 |
| B3GNT8 | Chr19: 46624975-46627975 | B3GNT8, Chr19: 46624975-46627975 |
| KANK4 | Chr1: 62556171-62559171 | KANK4, Chr1: 62556171-62559171 |
| MMP2 | Chr16: 54069081-54072081 | MMP2, Chr16: 54069081-54072081 |
| CNN2 | Chr19: 975797-978797 | CNN2, Chr19: 975797-978797 |
| S100A6 | Chr1: 151773841-151776841 | S100A6, Chr1: 151773841-151776841 |
| NNT | Chr5: 43637047-43640047 | NNT, Chr5: 43637047-43640047 |
| GLT25D2 | Chr1: 182271986-182274986 | GLT25D2, Chr1: 182271986-182274986 |
| CPT1B | Chr22: 49362362-49365362 | CPT1B, Chr22: 49362362-49365362 |
| NRXN1 | Chr2: 50426898-50429898 | NRXN1, Chr2: 50426898-50429898 |
| RAB42 | Chr1: 28789798-28792798 | RAB42, Chr1: 28789798-28792798 |
| TET1 | Chr10: 69988622-69991622 | TET1, Chr10: 69988622-69991622 |
| MDGA2 | Chr14: 47212238-47215238 | MDGA2, Chr14: 47212238-47215238 |
| HOXC4 | Chr12: 52732427-52735427 | HOXC4, Chr12: 52732427-52735427 |
| THPO | Chr3: 185577126-185580126 | THPO, Chr3: 185577126-185580126 |
| SIX2 | Chr2: 45088526-45091526 | SIX2, Chr2: 45088526-45091526 |
| KIAA1754L | Chr2: 96354187-96357187 | KIAA1754L, Chr2: 96354187-96357187 |
| RAMP3 | Chr7: 45162391-45165391 | RAMP3, Chr7: 45162391-45165391 |
| CHAT | Chr10: 50489659-50492659 | CHAT, Chr10: 50489659-50492659 |
| GRIP2 | Chr3: 14557092-14560092 | GRIP2, Chr3: 14557092-14560092 |
| IKBKG | Chr23: 153422152-153425152 | IKBKG, Chr23: 153422152-153425152 |
| MT1H | Chr16: 55259726-55262726 | MT1H, Chr16: 55259726-55262726 |
| CHAT | Chr10: 50490588-50493588 | CHAT, Chr10: 50490588-50493588 |
| LILRA2 | Chr19: 59775570-59778570 | LILRA2, Chr19: 59775570-59778570 |
| ADAMTS1 | Chr21: 27138099-27141099 | ADAMTS1, Chr21: 27138099-27141099 |
| NOS3 | Chr7: 150317579-150320579 | NOS3, Chr7: 150317579-150320579 |
| IGF2BP2 | Chr3: 187024021-187027021 | IGF2BP2, Chr3: 187024021-187027021 |
| FLJ35767 | Chr17: 77908912-77911912 | FLJ35767, Chr17: 77908912-77911912 |
| LYL1 | Chr19: 13073181-13076181 | LYL1, Chr19: 13073181-13076181 |
| HSP90AB1 | Chr6: 44321326-44324326 | HSP90AB1, Chr6: 44321326-44324326 |
| RARG | Chr12: 51910803-51913803 | RARG, Chr12: 51910803-51913803 |
| PLEKHB1 | Chr11: 73034741-73037741 | PLEKHB1, Chr11: 73034741-73037741 |
| TENC1 | Chr12: 51728601-51731601 | TENC1, Chr12: 51728601-51731601 |
| LRRC29 | Chr16: 65816722-65819722 | LRRC29, Chr16: 65816722-65819722 |
| GMIP | Chr19: 19613955-19616955 | GMIP, Chr19: 19613955-19616955 |
| GAA | Chr17: 75688449-75691449 | GAA, Chr17: 75688449-75691449 |
| TMEM176A | Chr7: 150127286-150130286 | TMEM176A, Chr7: 150127286-150130286 |
| SLC18A3 | Chr10: 50486852-50489852 | SLC18A3, Chr10: 50486852-50489852 |
| CRYAB | Chr11: 111286183-111289183 | CRYAB, Chr11: 111286183-111289183 |
| PYY | Chr17: 39435863-39438863 | PYY, Chr17: 39435863-39438863 |
| MMP2 | Chr16: 54071474-54074474 | MMP2, Chr16: 54071474-54074474 |
| TMEM176B | Chr7: 150125130-150128130 | TMEM176B, Chr7: 150125130-150128130 |
| CX3CR1 | Chr3: 39295031-39298031 | CX3CR1, Chr3: 39295031-39298031 |
| SGIP1 | Chr1: 66770912-66773912 | SGIP1, Chr1: 66770912-66773912 |
| SFTPH | Chr14: 36051241-36054241 | SFTPH, Chr14: 36051241-36054241 |
| MUSTN1 | Chr3: 52842760-52845760 | MUSTN1, Chr3: 52842760-52845760 |
| HOXD8 | Chr2: 176701222-176704222 | HOXD8, Chr2: 176701222-176704222 |
| TMEM85 | Chr15: 32303036-32306036 | TMEM85, Chr15: 32303036-32306036 |
| ZCCHC16 | Chr23: 111211408-111214408 | ZCCHC16, Chr23: 111211408-111214408 |
| TMEM176B | Chr7: 150127054-150130054 | TMEM176B, Chr7: 150127054-150130054 |
| DPP6 | Chr7: 153213851-153216851 | DPP6, Chr7: 153213851-153216851 |
| KIF1C | Chr17: 4840499-4843499 | KIF1C, Chr17: 4840499-4843499 |
| SDCCAG1 | Chr14: 49387789-49390789 | SDCCAG1, Chr14: 49387789-49390789 |
| NID2 | Chr14: 51604196-51607196 | NID2, Chr14: 51604196-51607196 |
| MYL9 | Chr20: 34601810-34604810 | MYL9, Chr20: 34601810-34604810 |
| NEUROD1 | Chr2: 182252126-182255126 | NEUROD1, Chr2: 182252126-182255126 |
| SLC10A7 | Chr4: 147660986-147663986 | SLC10A7, Chr4: 147660986-147663986 |
| TC2N | Chr14: 91371102-91374102 | TC2N, Chr14: 91371102-91374102 |
| SLC25A39 | Chr17: 39756158-39759158 | SLC25A39, Chr17: 39756158-39759158 |
| POU4F2 | Chr4: 147777994-147780994 | POU4F2, Chr4: 147777994-147780994 |
| SLC13A5 | Chr17: 6555888-6558888 | SLC13A5, Chr17: 6555888-6558888 |
| CABP1 | Chr12: 119571253-119574253 | CABP1, Chr12: 119571253-119574253 |
| ENO3 | Chr17: 4793630-4796630 | ENO3, Chr17: 4793630-4796630 |
| PLEKHG5 | Chr1: 6472847-6475847 | PLEKHG5, Chr1: 6472847-6475847 |
| C14orf159 | Chr14: 90649231-90652231 | C14orf159, Chr14: 90649231-90652231 |
| TDGF1 | Chr3: 46592716-46595716 | TDGF1, Chr3: 46592716-46595716 |
| C1orf220 | Chr1: 176777053-176780053 | C1orf220, Chr1: 176777053-176780053 |
| SLC10A7 | Chr4: 147661035-147664035 | SLC10A7, Chr4: 147661035-147664035 |
| GRIN2D | Chr19: 53588443-53591443 | GRIN2D, Chr19: 53588443-53591443 |
| CHRM2 | Chr7: 136203023-136206023 | CHRM2, Chr7: 136203023-136206023 |
| CHRM2 | Chr7: 136203032-136206032 | CHRM2, Chr7: 136203032-136206032 |
| AGBL2 | Chr11: 47691378-47694378 | AGBL2, Chr11: 47691378-47694378 |
| SLFN11 | Chr17: 30723333-30726333 | SLFN11, Chr17: 30723333-30726333 |
| ANXA2 | Chr15: 58475977-58478977 | ANXA2, Chr15: 58475977-58478977 |
| ZMYND8 | Chr20: 45417381-45420381 | ZMYND8, Chr20: 45417381-45420381 |
| CHRM2 | Chr7: 136202908-136205908 | CHRM2, Chr7: 136202908-136205908 |
| ECEL1 | Chr2: 233059276-233062276 | ECEL1, Chr2: 233059276-233062276 |
| ADCY4 | Chr14: 23872204-23875204 | ADCY4, Chr14: 23872204-23875204 |
| C18orf1 | Chr18: 13600165-13603165 | C18orf1, Chr18: 13600165-13603165 |
| KIAA1754L | Chr2: 96353372-96356372 | KIAA1754L, Chr2: 96353372-96356372 |
| ACSF2 | Chr17: 45857017-45860017 | ACSF2, Chr17: 45857017-45860017 |
| LOC339047 | Chr16: 16331734-16334734 | LOC339047, Chr16: 16331734-16334734 |
| MGC34761 | Chr16: 68763928-68766928 | MGC34761, Chr16: 68763928-68766928 |
| TWIST1 | Chr7: 19122320-19125320 | TWIST1, Chr7: 19122320-19125320 |
| COL8A1 | Chr3: 100838643-100841643 | COL8A1, Chr3: 100838643-100841643 |
| BRD4 | Chr19: 15250762-15253762 | BRD4, Chr19: 15250762-15253762 |
| CHRM2 | Chr7: 136202871-136205871 | CHRM2, Chr7: 136202871-136205871 |
| ZFHX4 | Chr8: 77754569-77757569 | ZFHX4, Chr8: 77754569-77757569 |
| TFAP2A | Chr6: 10526283-10529283 | TFAP2A, Chr6: 10526283-10529283 |
| ACOT11 | Chr1: 54784988-54787988 | ACOT11, Chr1: 54784988-54787988 |
| C1orf49 | Chr1: 176747334-176750334 | C1orf49, Chr1: 176747334-176750334 |
| SLC16A11 | Chr17: 6886466-6889466 | SLC16A11, Chr17: 6886466-6889466 |
| ADAMTS20 | Chr12: 42230491-42233491 | ADAMTS20, Chr12: 42230491-42233491 |
| FBXO39 | Chr17: 6618795-6621795 | FBXO39, Chr17: 6618795-6621795 |
| FGF12 | Chr3: 193607206-193610206 | FGF12, Chr3: 193607206-193610206 |
| DOCK2 | Chr5: 168995370-168998370 | DOCK2, Chr5: 168995370-168998370 |
| WDR69 | Chr2: 228443070-228446070 | WDR69, Chr2: 228443070-228446070 |
| ME3 | Chr11: 86059388-86062388 | ME3, Chr11: 86059388-86062388 |
| GLIPR1L1 | Chr12: 74013229-74016229 | GLIPR1L1, Chr12: 74013229-74016229 |
| CA5B | Chr23: 15664832-15667832 | CA5B, Chr23: 15664832-15667832 |
| SYNPR | Chr3: 63237453-63240453 | SYNPR, Chr3: 63237453-63240453 |
| PCDHGC4 | Chr5: 140843424-140846424 | PCDHGC4, Chr5: 140843424-140846424 |
| MYBPHL | Chr1: 109649686-109652686 | MYBPHL, Chr1: 109649686-109652686 |
| RUSC1 | Chr1: 153555763-153558763 | RUSC1, Chr1: 153555763-153558763 |
| GDAP1L1 | Chr20: 42307821-42310821 | GDAP1L1, Chr20: 42307821-42310821 |
| RPL39L | Chr3: 188338457-188341457 | RPL39L, Chr3: 188338457-188341457 |
| C1orf190 | Chr1: 46440092-46443092 | C1orf190, Chr1: 46440092-46443092 |
| FAM127C | Chr23: 133982732-133985732 | FAM127C, Chr23: 133982732-133985732 |
| QPCT | Chr2: 37423756-37426756 | QPCT, Chr2: 37423756-37426756 |
| APC | Chr5: 112099954-112102954 | APC, Chr5: 112099954-112102954 |
| SCG5 | Chr15: 30719751-30722751 | SCG5, Chr15: 30719751-30722751 |
| LST1 | Chr6: 31661103-31664103 | LST1, Chr6: 31661103-31664103 |
| SEC14L3 | Chr22: 29196517-29199517 | SEC14L3, Chr22: 29196517-29199517 |
| ELF3 | Chr1: 200244812-200247812 | ELF3, Chr1: 200244812-200247812 |
| TMEM176B | Chr7: 150127881-150130881 | TMEM176B, Chr7: 150127881-150130881 |
| CD8A | Chr2: 86870138-86873138 | CD8A, Chr2: 86870138-86873138 |
| FLJ36070 | Chr19: 53910526-53913526 | FLJ36070, Chr19: 53910526-53913526 |
| ANKRD53 | Chr2: 71057582-71060582 | ANKRD53, Chr2: 71057582-71060582 |
| KIF5A | Chr12: 56228613-56231613 | KIF5A, Chr12: 56228613-56231613 |
| ME3 | Chr11: 86059575-86062575 | ME3, Chr11: 86059575-86062575 |
| DHRS4L2 | Chr14: 23526366-23529366 | DHRS4L2, Chr14: 23526366-23529366 |
| GSTM2 | Chr1: 110010713-110013713 | GSTM2, Chr1: 110010713-110013713 |
| TRPV5 | Chr7: 142339442-142342442 | TRPV5, Chr7: 142339442-142342442 |
| SLC7A3 | Chr23: 70066184-70069184 | SLC7A3, Chr23: 70066184-70069184 |
| WDR52 | Chr3: 114641530-114644530 | WDR52, Chr3: 114641530-114644530 |
| SLC38A4 | Chr12: 45504502-45507502 | SLC38A4, Chr12: 45504502-45507502 |
| ICHTHYIN | Chr5: 156818104-156821104 | ICHTHYIN, Chr5: 156818104-156821104 |
| C6orf206 | Chr6: 43719287-43722287 | C6orf206, Chr6: 43719287-43722287 |
| GTF2A1L | Chr2: 48696951-48699951 | GTF2A1L, Chr2: 48696951-48699951 |
| C14orf159 | Chr14: 90648609-90651609 | C14orf159, Chr14: 90648609-90651609 |
| FOXR1 | Chr11: 118346126-118349126 | FOXR1, Chr11: 118346126-118349126 |
| LAMA3 | Chr18: 19705481-19708481 | LAMA3, Chr18: 19705481-19708481 |
| KLK13 | Chr19: 56258679-56261679 | KLK13, Chr19: 56258679-56261679 |
| CPLX3 | Chr15: 72904503-72907503 | CPLX3, Chr15: 72904503-72907503 |
| CPNE4 | Chr3: 133235034-133238034 | CPNE4, Chr3: 133235034-133238034 |
| CBX5 | Chr12: 52938096-52941096 | CBX5, Chr12: 52938096-52941096 |
| KLHL31 | Chr6: 53636965-53639965 | KLHL31, Chr6: 53636965-53639965 |
| CHRM2 | Chr7: 136202438-136205438 | CHRM2, Chr7: 136202438-136205438 |
| PTGDR | Chr14: 51802680-51805680 | PTGDR, Chr14: 51802680-51805680 |
| LTF | Chr3: 46479899-46482899 | LTF, Chr3: 46479899-46482899 |
| NFAM1 | Chr22: 41156845-41159845 | NFAM1, Chr22: 41156845-41159845 |
| CX3CL1 | Chr16: 55962414-55965414 | CX3CL1, Chr16: 55962414-55965414 |
| KHDRBS2 | Chr6: 63052559-63055559 | KHDRBS2, Chr6: 63052559-63055559 |
| PDGFRB | Chr5: 149514115-149517115 | PDGFRB, Chr5: 149514115-149517115 |
| CXCL5 | Chr4: 75081780-75084780 | CXCL5, Chr4: 75081780-75084780 |
| KRT81 | Chr12: 50970066-50973066 | KRT81, Chr12: 50970066-50973066 |
| PDCL2 | Chr4: 56151636-56154636 | PDCL2, Chr4: 56151636-56154636 |
| CPVL | Chr7: 29151178-29154178 | CPVL, Chr7: 29151178-29154178 |
| ELTD1 | Chr1: 79243583-79246583 | ELTD1, Chr1: 79243583-79246583 |
| ZNF660 | Chr3: 44599959-44602959 | ZNF660, Chr3: 44599959-44602959 |
| NRSN1 | Chr6: 24232892-24235892 | NRSN1, Chr6: 24232892-24235892 |
| HIST1H3C | Chr6: 26152117-26155117 | HIST1H3C, Chr6: 26152117-26155117 |
| TRPV2 | Chr17: 16258112-16261112 | TRPV2, Chr17: 16258112-16261112 |
| TRAK1 | Chr3: 42175164-42178164 | TRAK1, Chr3: 42175164-42178164 |
| C10orf53 | Chr10: 50556189-50559189 | C10orf53, Chr10: 50556189-50559189 |
| EIF2AK3 | Chr2: 88706609-88709609 | EIF2AK3, Chr2: 88706609-88709609 |
| DENND1C | Chr19: 6431298-6434298 | DENND1C, Chr19: 6431298-6434298 |
| CHAT | Chr10: 50485646-50488646 | CHAT, Chr10: 50485646-50488646 |
| C15orf26 | Chr15: 79212198-79215198 | C15orf26, Chr15: 79212198-79215198 |
| MITF | Chr3: 70009445-70012445 | MITF, Chr3: 70009445-70012445 |
| SERPINA5 | Chr14: 94115983-94118983 | SERPINA5, Chr14: 94115983-94118983 |
| COL11A1 | Chr1: 103345140-103348140 | COL11A1, Chr1: 103345140-103348140 |
| LAIR2 | Chr19: 59704324-59707324 | LAIR2, Chr19: 59704324-59707324 |
| CBX5 | Chr12: 52938137-52941137 | CBX5, Chr12: 52938137-52941137 |
| LY9 | Chr1: 159031051-159034051 | LY9, Chr1: 159031051-159034051 |
| F13A1 | Chr6: 6264423-6267423 | F13A1, Chr6: 6264423-6267423 |
| SLCO4C1 | Chr5: 101658652-101661652 | SLCO4C1, Chr5: 101658652-101661652 |
| COL9A1 | Chr6: 71048132-71051132 | COL9A1, Chr6: 71048132-71051132 |
| FAM70A | Chr23: 119327919-119330919 | FAM70A, Chr23: 119327919-119330919 |
| ALOX15B | Chr17: 7881582-7884582 | ALOX15B, Chr17: 7881582-7884582 |
| CD40 | Chr20: 44178812-44181812 | CD40, Chr20: 44178812-44181812 |
| PDE2A | Chr11: 72061560-72064560 | PDE2A, Chr11: 72061560-72064560 |
| OXGR1 | Chr13: 96443105-96446105 | OXGR1, Chr13: 96443105-96446105 |
| SACS | Chr13: 22904341-22907341 | SACS, Chr13: 22904341-22907341 |
| LBP | Chr20: 36406798-36409798 | LBP, Chr20: 36406798-36409798 |
| S100A16 | Chr1: 151850638-151853638 | S100A16, Chr1: 151850638-151853638 |
| MYL1 | Chr2: 210886640-210889640 | MYL1, Chr2: 210886640-210889640 |
| HFE | Chr6: 26193987-26196987 | HFE, Chr6: 26193987-26196987 |
| LRRC15 | Chr3: 195570261-195573261 | LRRC15, Chr3: 195570261-195573261 |
| HIST1H2BE | Chr6: 26290502-26293502 | HIST1H2BE, Chr6: 26290502-26293502 |
| WBSCR28 | Chr7: 72911924-72914924 | WBSCR28, Chr7: 72911924-72914924 |
| CIB3 | Chr19: 16143786-16146786 | CIB3, Chr19: 16143786-16146786 |
| UCN3 | Chr10: 5395475-5398475 | UCN3, Chr10: 5395475-5398475 |
| PF4V1 | Chr4: 74936376-74939376 | PF4V1, Chr4: 74936376-74939376 |
| LGALS2 | Chr22: 36304470-36307470 | LGALS2, Chr22: 36304470-36307470 |
| CKMT2 | Chr5: 80563394-80566394 | CKMT2, Chr5: 80563394-80566394 |
| CD33 | Chr19: 56418646-56421646 | CD33, Chr19: 56418646-56421646 |
| CXCL6 | Chr4: 74919776-74922776 | CXCL6, Chr4: 74919776-74922776 |
| PLEKHB1 | Chr11: 73033370-73036370 | PLEKHB1, Chr11: 73033370-73036370 |
| C6orf103 | Chr6: 146960328-146963328 | C6orf103, Chr6: 146960328-146963328 |
| PRSS3 | Chr9: 33739014-33742014 | PRSS3, Chr9: 33739014-33742014 |
| CD177 | Chr19: 48548164-48551164 | CD177, Chr19: 48548164-48551164 |
| EGFL6 | Chr23: 13496161-13499161 | EGFL6, Chr23: 13496161-13499161 |
| PAH | Chr12: 101834011-101837011 | PAH, Chr12: 101834011-101837011 |
| LAMC2 | Chr1: 181420296-181423296 | LAMC2, Chr1: 181420296-181423296 |
| HMGCLL1 | Chr6: 55550471-55553471 | HMGCLL1, Chr6: 55550471-55553471 |
| SLC16A13 | Chr17: 6878925-6881925 | SLC16A13, Chr17: 6878925-6881925 |
| RGN | Chr23: 46821218-46824218 | RGN, Chr23: 46821218-46824218 |
| MAMDC2 | Chr9: 71846839-71849839 | MAMDC2, Chr9: 71846839-71849839 |
| HDDC3 | Chr15: 89275278-89278278 | HDDC3, Chr15: 89275278-89278278 |
| ENTPD3 | Chr3: 40402176-40405176 | ENTPD3, Chr3: 40402176-40405176 |
| TEKT5 | Chr16: 10694803-10697803 | TEKT5, Chr16: 10694803-10697803 |
| PGAM4 | Chr23: 77110291-77113291 | PGAM4, Chr23: 77110291-77113291 |
| SYNPO2L | Chr10: 75084338-75087338 | SYNPO2L, Chr10: 75084338-75087338 |
| PAX4 | Chr7: 127041516-127044516 | PAX4, Chr7: 127041516-127044516 |
| MEOX1 | Chr17: 39092957-39095957 | MEOX1, Chr17: 39092957-39095957 |
| CTRB1 | Chr16: 73808884-73811884 | CTRB1, Chr16: 73808884-73811884 |
| ULBP2 | Chr6: 150303328-150306328 | ULBP2, Chr6: 150303328-150306328 |
| KCNAB1 | Chr3: 157489969-157492969 | KCNAB1, Chr3: 157489969-157492969 |
| PTGER2 | Chr14: 51849362-51852362 | PTGER2, Chr14: 51849362-51852362 |
| RND1 | Chr12: 47544420-47547420 | RND1, Chr12: 47544420-47547420 |
| LY75 | Chr2: 160468008-160471008 | LY75, Chr2: 160468008-160471008 |
| FLJ21438 | Chr19: 15434882-15437882 | FLJ21438, Chr19: 15434882-15437882 |
| TMLHE | Chr23: 154494291-154497291 | TMLHE, Chr23: 154494291-154497291 |
| MAGEA10 | Chr23: 151056181-151059181 | MAGEA10, Chr23: 151056181-151059181 |
| ATP6V0A4 | Chr7: 138107822-138110822 | ATP6V0A4, Chr7: 138107822-138110822 |
| ABP1 | Chr7: 150179005-150182005 | ABP1, Chr7: 150179005-150182005 |
| HNRNPA3 | Chr2: 177784167-177787167 | HNRNPA3, Chr2: 177784167-177787167 |
| SH3TC2 | Chr5: 148421430-148424430 | SH3TC2, Chr5: 148421430-148424430 |
| KRTAP3-1 | Chr17: 36417392-36420392 | KRTAP3-1, Chr17: 36417392-36420392 |
| RAB27B | Chr18: 50645337-50648337 | RAB27B, Chr18: 50645337-50648337 |
| MEOX2 | Chr7: 15691333-15694333 | MEOX2, Chr7: 15691333-15694333 |
| S100A4 | Chr1: 151783406-151786406 | S100A4, Chr1: 151783406-151786406 |
| CCDC48 | Chr3: 130230481-130233481 | CCDC48, Chr3: 130230481-130233481 |
| CCDC13 | Chr3: 42788249-42791249 | CCDC13, Chr3: 42788249-42791249 |
| PAGE4 | Chr23: 49479143-49482143 | PAGE4, Chr23: 49479143-49482143 |
| B3GNT3 | Chr19: 17765418-17768418 | B3GNT3, Chr19: 17765418-17768418 |
| CLIP3 | Chr19: 41214115-41217115 | CLIP3, Chr19: 41214115-41217115 |
| IKZF4 | Chr12: 54699455-54702455 | IKZF4, Chr12: 54699455-54702455 |
| LDHB | Chr12: 21700543-21703543 | LDHB, Chr12: 21700543-21703543 |
| SERPINA6 | Chr14: 93857941-93860941 | SERPINA6, Chr14: 93857941-93860941 |
| KRT76 | Chr12: 51455872-51458872 | KRT76, Chr12: 51455872-51458872 |
| CST4 | Chr20: 23616162-23619162 | CST4, Chr20: 23616162-23619162 |
| CHST8 | Chr19: 38865773-38868773 | CHST8, Chr19: 38865773-38868773 |
| AGTRAP | Chr1: 11717228-11720228 | AGTRAP, Chr1: 11717228-11720228 |
| ESM1 | Chr5: 54315667-54318667 | ESM1, Chr5: 54315667-54318667 |
| MLXIPL | Chr7: 72675306-72678306 | MLXIPL, Chr7: 72675306-72678306 |
| SHC1 | Chr1: 153208347-153211347 | SHC1, Chr1: 153208347-153211347 |
| INCA1 | Chr17: 4840129-4843129 | INCA1, Chr17: 4840129-4843129 |
| RBP5 | Chr12: 7171233-7174233 | RBP5, Chr12: 7171233-7174233 |
| LPCAT2 | Chr16: 54098954-54101954 | LPCAT2, Chr16: 54098954-54101954 |
| LST1 | Chr6: 31660449-31663449 | LST1, Chr6: 31660449-31663449 |
| TNFSF13B | Chr13: 107718477-107721477 | TNFSF13B, Chr13: 107718477-107721477 |
| MEOX1 | Chr17: 39093288-39096288 | MEOX1, Chr17: 39093288-39096288 |
| JAK3 | Chr19: 17818341-17821341 | JAK3, Chr19: 17818341-17821341 |
| CHD3 | Chr17: 7731393-7734393 | CHD3, Chr17: 7731393-7734393 |
| OGDHL | Chr10: 50638875-50641875 | OGDHL, Chr10: 50638875-50641875 |
| HOXD3 | Chr2: 176735550-176738550 | HOXD3, Chr2: 176735550-176738550 |
| NAGS | Chr17: 39436057-39439057 | NAGS, Chr17: 39436057-39439057 |
| PYGL | Chr14: 50479484-50482484 | PYGL, Chr14: 50479484-50482484 |
| LSAMP | Chr3: 117645568-117648568 | LSAMP, Chr3: 117645568-117648568 |
| HPS1 | Chr10: 100195194-100198194 | HPS1, Chr10: 100195194-100198194 |
| HSPB8 | Chr12: 118099477-118102477 | HSPB8, Chr12: 118099477-118102477 |
| RBM12 | Chr20: 33714752-33717752 | RBM12, Chr20: 33714752-33717752 |
| CPNE1 | Chr20: 33714762-33717762 | CPNE1, Chr20: 33714762-33717762 |
| TIMP4 | Chr3: 12174147-12177147 | TIMP4, Chr3: 12174147-12177147 |
| RCN3 | Chr19: 54721186-54724186 | RCN3, Chr19: 54721186-54724186 |
| HOXD1 | Chr2: 176760052-176763052 | HOXD1, Chr2: 176760052-176763052 |
| MAGEC1 | Chr23: 140817845-140820845 | MAGEC1, Chr23: 140817845-140820845 |
| GDF15 | Chr19: 18356467-18359467 | GDF15, Chr19: 18356467-18359467 |
| GALNTL2 | Chr3: 16189687-16192687 | GALNTL2, Chr3: 16189687-16192687 |
| BEST4 | Chr1: 45024513-45027513 | BEST4, Chr1: 45024513-45027513 |
| TMEM196 | Chr7: 19778041-19781041 | TMEM196, Chr7: 19778041-19781041 |
| MAP1A | Chr15: 41595597-41598597 | MAP1A, Chr15: 41595597-41598597 |
| TRPC5 | Chr23: 111211160-111214160 | TRPC5, Chr23: 111211160-111214160 |
| PER1 | Chr17: 7994978-7997978 | PER1, Chr17: 7994978-7997978 |
| JAM2 | Chr21: 25931959-25934959 | JAM2, Chr21: 25931959-25934959 |
| NPTX1 | Chr17: 76063499-76066499 | NPTX1, Chr17: 76063499-76066499 |
| CD209 | Chr19: 7716906-7719906 | CD209, Chr19: 7716906-7719906 |
| JMJD1A | Chr2: 86520364-86523364 | JMJD1A, Chr2: 86520364-86523364 |
| PITPNM3 | Chr17: 6399101-6402101 | PITPNM3, Chr17: 6399101-6402101 |
| FERD3L | Chr7: 19150069-19153069 | FERD3L, Chr7: 19150069-19153069 |
| RDH5 | Chr12: 54398987-54401987 | RDH5, Chr12: 54398987-54401987 |
| CALHM2 | Chr10: 105200565-105203565 | CALHM2, Chr10: 105200565-105203565 |
| KIAA0467 | Chr1: 43659883-43662883 | KIAA0467, Chr1: 43659883-43662883 |
| UBD | Chr6: 29634181-29637181 | UBD, Chr6: 29634181-29637181 |
| ITGAL | Chr16: 30389983-30392983 | ITGAL, Chr16: 30389983-30392983 |
| NHLH2 | Chr1: 116183770-116186770 | NHLH2, Chr1: 116183770-116186770 |
| LMO3 | Chr12: 16649198-16652198 | LMO3, Chr12: 16649198-16652198 |
| GJA1 | Chr6: 121796943-121799943 | GJA1, Chr6: 121796943-121799943 |
| LOC728635 | Chr14: 23574049-23577049 | LOC728635, Chr14: 23574049-23577049 |
| BAK1 | Chr6: 33654548-33657548 | BAK1, Chr6: 33654548-33657548 |
| SH3RF2 | Chr5: 145294834-145297834 | SH3RF2, Chr5: 145294834-145297834 |
| MAML2 | Chr11: 95714492-95717492 | MAML2, Chr11: 95714492-95717492 |
| STK24 | Chr13: 97970842-97973842 | STK24, Chr13: 97970842-97973842 |
| ELAVL2 | Chr9: 23814563-23817563 | ELAVL2, Chr9: 23814563-23817563 |
| PCDH20 | Chr13: 60886156-60889156 | PCDH20, Chr13: 60886156-60889156 |
| PRDM16 | Chr1: 2974103-2977103 | PRDM16, Chr1: 2974103-2977103 |
| MNT | Chr17: 2249508-2252508 | MNT, Chr17: 2249508-2252508 |
| HPCAL1 | Chr2: 10358990-10361990 | HPCAL1, Chr2: 10358990-10361990 |
| KCNK9 | Chr8: 140782981-140785981 | KCNK9, Chr8: 140782981-140785981 |
| CTBP2 | Chr10: 126838114-126841114 | CTBP2, Chr10: 126838114-126841114 |
| BCL2L11 | Chr2: 111593461-111596461 | BCL2L11, Chr2: 111593461-111596461 |
| FZD10 | Chr12: 129211484-129214484 | FZD10, Chr12: 129211484-129214484 |
| LOC728661 | Chr1: 1612603-1615603 | LOC728661, Chr1: 1612603-1615603 |
| TBX2 | Chr17: 56830538-56833538 | TBX2, Chr17: 56830538-56833538 |
| NOC2L | Chr1: 883042-886042 | NOC2L, Chr1: 883042-886042 |
| FBXW9 | Chr19: 12666955-12669955 | FBXW9, Chr19: 12666955-12669955 |
| FOXI2 | Chr10: 129424444-129427444 | FOXI2, Chr10: 129424444-129427444 |
| RASGRF1 | Chr15: 77168395-77171395 | RASGRF1, Chr15: 77168395-77171395 |
| GFRA1 | Chr10: 118020284-118023284 | GFRA1, Chr10: 118020284-118023284 |
| NTRK2 | Chr9: 86472945-86475945 | NTRK2, Chr9: 86472945-86475945 |
| RAP2B | Chr3: 154361218-154364218 | RAP2B, Chr3: 154361218-154364218 |
| CENTB1 | Chr17: 7179096-7182096 | CENTB1, Chr17: 7179096-7182096 |
| MIDN | Chr19: 1198051-1201051 | MIDN, Chr19: 1198051-1201051 |
| DVL1 | Chr1: 1272855-1275855 | DVL1, Chr1: 1272855-1275855 |
| DPYSL4 | Chr10: 133848903-133851903 | DPYSL4, Chr10: 133848903-133851903 |
| SHC2 | Chr19: 410496-413496 | SHC2, Chr19: 410496-413496 |
| HPCAL1 | Chr2: 10359776-10362776 | HPCAL1, Chr2: 10359776-10362776 |
| GRHL1 | Chr2: 10007772-10010772 | GRHL1, Chr2: 10007772-10010772 |
| MRPS26 | Chr20: 2973174-2976174 | MRPS26, Chr20: 2973174-2976174 |
| ITPKB | Chr1: 224990318-224993318 | ITPKB, Chr1: 224990318-224993318 |
| GRIN2C | Chr17: 70366102-70369102 | GRIN2C, Chr17: 70366102-70369102 |
| C2CD2 | Chr21: 42245568-42248568 | C2CD2, Chr21: 42245568-42248568 |
| RTKN | Chr2: 74521068-74524068 | RTKN, Chr2: 74521068-74524068 |
| FGF11 | Chr17: 7281912-7284912 | FGF11, Chr17: 7281912-7284912 |
| LOC91461 | Chr2: 42127756-42130756 | LOC91461, Chr2: 42127756-42130756 |
| BAD | Chr11: 63807240-63810240 | BAD, Chr11: 63807240-63810240 |
| DKK3 | Chr11: 11985262-11988262 | DKK3, Chr11: 11985262-11988262 |
| HAS3 | Chr16: 67697443-67700443 | HAS3, Chr16: 67697443-67700443 |
| RPL28 | Chr19: 60587611-60590611 | RPL28, Chr19: 60587611-60590611 |
| NKX2-8 | Chr14: 36120037-36123037 | NKX2-8, Chr14: 36120037-36123037 |
| BAD | Chr11: 63807157-63810157 | BAD, Chr11: 63807157-63810157 |
| KIF26A | Chr14: 103673312-103676312 | KIF26A, Chr14: 103673312-103676312 |
| MPST | Chr22: 35744147-35747147 | MPST, Chr22: 35744147-35747147 |
| IL4I1 | Chr19: 55123074-55126074 | IL4I1, Chr19: 55123074-55126074 |
| NUP62 | Chr19: 55123098-55126098 | NUP62, Chr19: 55123098-55126098 |
| RETN | Chr19: 7638471-7641471 | RETN, Chr19: 7638471-7641471 |
| FOXA2 | Chr20: 22512602-22515602 | FOXA2, Chr20: 22512602-22515602 |
| EDF1 | Chr9: 138879059-138882059 | EDF1, Chr9: 138879059-138882059 |
| DNAI1 | Chr9: 34447310-34450310 | DNAI1, Chr9: 34447310-34450310 |
| KCND3 | Chr1: 112331800-112334800 | KCND3, Chr1: 112331800-112334800 |
| DMPK | Chr19: 50974201-50977201 | DMPK, Chr19: 50974201-50977201 |
| TTC15 | Chr2: 3360952-3363952 | TTC15, Chr2: 3360952-3363952 |
| FOXA3 | Chr19: 51057857-51060857 | FOXA3, Chr19: 51057857-51060857 |
| GAK | Chr4: 914674-917674 | GAK, Chr4: 914674-917674 |
| CPXM1 | Chr20: 2727782-2730782 | CPXM1, Chr20: 2727782-2730782 |
| MDFI | Chr6: 41712672-41715672 | MDFI, Chr6: 41712672-41715672 |
| SLC16A5 | Chr17: 70594149-70597149 | SLC16A5, Chr17: 70594149-70597149 |
| ATP6V0C | Chr16: 2502453-2505453 | ATP6V0C, Chr16: 2502453-2505453 |
| FOXL1 | Chr16: 85168115-85171115 | FOXL1, Chr16: 85168115-85171115 |
| TINP1 | Chr5: 74097358-74100358 | TINP1, Chr5: 74097358-74100358 |
| C17orf28 | Chr17: 70478995-70481995 | C17orf28, Chr17: 70478995-70481995 |
| VAX2 | Chr2: 70979727-70982727 | VAX2, Chr2: 70979727-70982727 |
| ZMYND15 | Chr17: 4588585-4591585 | ZMYND15, Chr17: 4588585-4591585 |
| PAQR4 | Chr16: 2957842-2960842 | PAQR4, Chr16: 2957842-2960842 |
| PLEKHF1 | Chr19: 34846666-34849666 | PLEKHF1, Chr19: 34846666-34849666 |
| ZNF461 | Chr19: 41848079-41851079 | ZNF461, Chr19: 41848079-41851079 |
| PEMT | Chr17: 17434219-17437219 | PEMT, Chr17: 17434219-17437219 |
| TAF8 | Chr6: 42124728-42127728 | TAF8, Chr6: 42124728-42127728 |
| EHBP1L1 | Chr11: 65098584-65101584 | EHBP1L1, Chr11: 65098584-65101584 |
| VILL | Chr3: 38008581-38011581 | VILL, Chr3: 38008581-38011581 |
| PRPF6 | Chr20: 62081374-62084374 | PRPF6, Chr20: 62081374-62084374 |
| CCDC120 | Chr23: 48801959-48804959 | CCDC120, Chr23: 48801959-48804959 |
| EXOSC2 | Chr9: 132557478-132560478 | EXOSC2, Chr9: 132557478-132560478 |
| ISL2 | Chr15: 74414701-74417701 | ISL2, Chr15: 74414701-74417701 |
| CCDC8 | Chr19: 51607181-51610181 | CCDC8, Chr19: 51607181-51610181 |
| PEAR1 | Chr1: 155128646-155131646 | PEAR1, Chr1: 155128646-155131646 |
| NFKB2 | Chr10: 104143921-104146921 | NFKB2, Chr10: 104143921-104146921 |
| RTKN | Chr2: 74519718-74522718 | RTKN, Chr2: 74519718-74522718 |
| CYP1A1 | Chr15: 72803430-72806430 | CYP1A1, Chr15: 72803430-72806430 |
| DFNB31 | Chr9: 116303816-116306816 | DFNB31, Chr9: 116303816-116306816 |
| GNG3 | Chr11: 62230208-62233208 | GNG3, Chr11: 62230208-62233208 |
| SNCA | Chr4: 90974354-90977354 | SNCA, Chr4: 90974354-90977354 |
| DGKZ | Chr11: 46322062-46325062 | DGKZ, Chr11: 46322062-46325062 |
| KLF16 | Chr19: 1813064-1816064 | KLF16, Chr19: 1813064-1816064 |
| ZFPM1 | Chr16: 87046014-87049014 | ZFPM1, Chr16: 87046014-87049014 |
| IL17RA | Chr22: 15944348-15947348 | IL17RA, Chr22: 15944348-15947348 |
| NUBP2 | Chr16: 1771433-1774433 | NUBP2, Chr16: 1771433-1774433 |
| FAM19A5 | Chr22: 47349281-47352281 | FAM19A5, Chr22: 47349281-47352281 |
| C19orf28 | Chr19: 3507071-3510071 | C19orf28, Chr19: 3507071-3510071 |
| KAZALD1 | Chr10: 102810087-102813087 | KAZALD1, Chr10: 102810087-102813087 |
| NRG3 | Chr10: 83623576-83626576 | NRG3, Chr10: 83623576-83626576 |
| TRIM60 | Chr4: 166171100-166174100 | TRIM60, Chr4: 166171100-166174100 |
| MST1 | Chr3: 49699599-49702599 | MST1, Chr3: 49699599-49702599 |
| KLK8 | Chr19: 56195270-56198270 | KLK8, Chr19: 56195270-56198270 |
| FAM177A1 | Chr14: 34582363-34585363 | FAM177A1, Chr14: 34582363-34585363 |
| ADRA1A | Chr8: 26777339-26780339 | ADRA1A, Chr8: 26777339-26780339 |
| NFIC | Chr19: 3309115-3312115 | NFIC, Chr19: 3309115-3312115 |
| FAM20C | Chr7: 286551-289551 | FAM20C, Chr7: 286551-289551 |
| PSMG3 | Chr7: 1574655-1577655 | PSMG3, Chr7: 1574655-1577655 |
| BRMS1L | Chr14: 35363847-35366847 | BRMS1L, Chr14: 35363847-35366847 |
| PLXNA4 | Chr7: 131910363-131913363 | PLXNA4, Chr7: 131910363-131913363 |
| CSTF3 | Chr11: 33138113-33141113 | CSTF3, Chr11: 33138113-33141113 |
| TTC16 | Chr9: 129516678-129519678 | TTC16, Chr9: 129516678-129519678 |
| RBM15B | Chr3: 51402270-51405270 | RBM15B, Chr3: 51402270-51405270 |
| GRID1 | Chr10: 88114730-88117730 | GRID1, Chr10: 88114730-88117730 |
| ABHD9 | Chr19: 15202731-15205731 | ABHD9, Chr19: 15202731-15205731 |
| SLC1A2 | Chr11: 35396181-35399181 | SLC1A2, Chr11: 35396181-35399181 |
| ANKRD39 | Chr2: 96885983-96888983 | ANKRD39, Chr2: 96885983-96888983 |
| ACSS3 | Chr12: 79994439-79997439 | ACSS3, Chr12: 79994439-79997439 |
| CCDC56 | Chr17: 38202730-38205730 | CCDC56, Chr17: 38202730-38205730 |
| TSSC1 | Chr2: 3359105-3362105 | TSSC1, Chr2: 3359105-3362105 |
| SUSD4 | Chr1: 221602667-221605667 | SUSD4, Chr1: 221602667-221605667 |
| TTC31 | Chr2: 74562223-74565223 | TTC31, Chr2: 74562223-74565223 |
| RPS6KL1 | Chr14: 74457398-74460398 | RPS6KL1, Chr14: 74457398-74460398 |
| MYH3 | Chr17: 10498690-10501690 | MYH3, Chr17: 10498690-10501690 |
| COL11A2 | Chr6: 33266723-33269723 | COL11A2, Chr6: 33266723-33269723 |
| P2RX6 | Chr22: 19697963-19700963 | P2RX6, Chr22: 19697963-19700963 |
| FGF18 | Chr5: 170777771-170780771 | FGF18, Chr5: 170777771-170780771 |
| CBLN1 | Chr16: 47871684-47874684 | CBLN1, Chr16: 47871684-47874684 |
| ADRA2A | Chr10: 112825410-112828410 | ADRA2A, Chr10: 112825410-112828410 |
| MICALL2 | Chr7: 1464135-1467135 | MICALL2, Chr7: 1464135-1467135 |
| LBH | Chr2: 30306400-30309400 | LBH, Chr2: 30306400-30309400 |
| FAM110C | Chr2: 34885-37885 | FAM110C, Chr2: 34885-37885 |
| ST8SIA1 | Chr12: 22377415-22380415 | ST8SIA1, Chr12: 22377415-22380415 |
| ZNF497 | Chr19: 63564432-63567432 | ZNF497, Chr19: 63564432-63567432 |
| TOX | Chr8: 60192821-60195821 | TOX, Chr8: 60192821-60195821 |
| DKK3 | Chr11: 11985705-11988705 | DKK3, Chr11: 11985705-11988705 |
| ELF4 | Chr23: 129070656-129073656 | ELF4, Chr23: 129070656-129073656 |
| ZFYVE21 | Chr14: 103250397-103253397 | ZFYVE21, Chr14: 103250397-103253397 |
| BANP | Chr16: 86541038-86544038 | BANP, Chr16: 86541038-86544038 |
| SYCE2 | Chr19: 12889586-12892586 | SYCE2, Chr19: 12889586-12892586 |
| PPAP2C | Chr19: 240669-243669 | PPAP2C, Chr19: 240669-243669 |
| YBX2 | Chr17: 7137100-7140100 | YBX2, Chr17: 7137100-7140100 |
| DLK1 | Chr14: 100261505-100264505 | DLK1, Chr14: 100261505-100264505 |
| TRAF2 | Chr9: 138899285-138902285 | TRAF2, Chr9: 138899285-138902285 |
| HRC | Chr19: 54348993-54351993 | HRC, Chr19: 54348993-54351993 |
| A3GALT2 | Chr1: 33557786-33560786 | A3GALT2, Chr1: 33557786-33560786 |
| TAF4 | Chr20: 60072761-60075761 | TAF4, Chr20: 60072761-60075761 |
| CHST2 | Chr3: 144319857-144322857 | CHST2, Chr3: 144319857-144322857 |
| GRIK3 | Chr1: 37270931-37273931 | GRIK3, Chr1: 37270931-37273931 |
| PATZ1 | Chr22: 30070749-30073749 | PATZ1, Chr22: 30070749-30073749 |
| RASGRP2 | Chr11: 64268004-64271004 | RASGRP2, Chr11: 64268004-64271004 |
| GPI | Chr19: 39546408-39549408 | GPI, Chr19: 39546408-39549408 |
| GPR124 | Chr8: 37772431-37775431 | GPR124, Chr8: 37772431-37775431 |
| SELO | Chr22: 48980034-48983034 | SELO, Chr22: 48980034-48983034 |
| CCNL2 | Chr1: 1323052-1326052 | CCNL2, Chr1: 1323052-1326052 |
| CCNL2 | Chr1: 1323053-1326053 | CCNL2, Chr1: 1323053-1326053 |
| ANKRD28 | Chr3: 15874557-15877557 | ANKRD28, Chr3: 15874557-15877557 |
| KCNS2 | Chr8: 99506925-99509925 | KCNS2, Chr8: 99506925-99509925 |
| TCP11L2 | Chr12: 105219210-105222210 | TCP11L2, Chr12: 105219210-105222210 |
| MTL5 | Chr11: 68274064-68277064 | MTL5, Chr11: 68274064-68277064 |
| PFKP | Chr10: 3098251-3101251 | PFKP, Chr10: 3098251-3101251 |
| DKK3 | Chr11: 11985993-11988993 | DKK3, Chr11: 11985993-11988993 |
| ELF4 | Chr23: 129070869-129073869 | ELF4, Chr23: 129070869-129073869 |
| KIAA1024 | Chr15: 77510412-77513412 | KIAA1024, Chr15: 77510412-77513412 |
| TMEM175 | Chr4: 914761-917761 | TMEM175, Chr4: 914761-917761 |
| HTRA1 | Chr10: 124209530-124212530 | HTRA1, Chr10: 124209530-124212530 |
| VPS28 | Chr8: 145623235-145626235 | VPS28, Chr8: 145623235-145626235 |
| UCK1 | Chr9: 133394976-133397976 | UCK1, Chr9: 133394976-133397976 |
| TMEM63B | Chr6: 44201853-44204853 | TMEM63B, Chr6: 44201853-44204853 |
| C10orf2 | Chr10: 102735801-102738801 | C10orf2, Chr10: 102735801-102738801 |
| ZFP42 | Chr4: 189152418-189155418 | ZFP42, Chr4: 189152418-189155418 |
| KATNAL1 | Chr13: 29777663-29780663 | KATNAL1, Chr13: 29777663-29780663 |
| C10orf141 | Chr10: 128882912-128885912 | C10orf141, Chr10: 128882912-128885912 |
| WNT3A | Chr1: 226259874-226262874 | WNT3A, Chr1: 226259874-226262874 |
| BMP7 | Chr20: 55273614-55276614 | BMP7, Chr20: 55273614-55276614 |
| ASCL2 | Chr11: 2247258-2250258 | ASCL2, Chr11: 2247258-2250258 |
| RPL36 | Chr19: 5639771-5642771 | RPL36, Chr19: 5639771-5642771 |
| NBL1 | Chr1: 19841894-19844894 | NBL1, Chr1: 19841894-19844894 |
| DOCK1 | Chr10: 128582512-128585512 | DOCK1, Chr10: 128582512-128585512 |
| MB | Chr22: 34341830-34344830 | MB, Chr22: 34341830-34344830 |
| ERRFI1 | Chr1: 8007443-8010443 | ERRFI1, Chr1: 8007443-8010443 |
| PNPLA6 | Chr19: 7503542-7506542 | PNPLA6, Chr19: 7503542-7506542 |
| PPAP2C | Chr19: 240935-243935 | PPAP2C, Chr19: 240935-243935 |
| SLC26A10 | Chr12: 56298459-56301459 | SLC26A10, Chr12: 56298459-56301459 |
| DCHS1 | Chr11: 6632150-6635150 | DCHS1, Chr11: 6632150-6635150 |
| LRWD1 | Chr7: 101890894-101893894 | LRWD1, Chr7: 101890894-101893894 |
| NXPH4 | Chr12: 55895344-55898344 | NXPH4, Chr12: 55895344-55898344 |
| ACSL1 | Chr4: 185982709-185985709 | ACSL1, Chr4: 185982709-185985709 |
| VIPR1 | Chr3: 42517620-42520620 | VIPR1, Chr3: 42517620-42520620 |
| FGF8 | Chr10: 103524317-103527317 | FGF8, Chr10: 103524317-103527317 |
| PDGFC | Chr4: 158110496-158113496 | PDGFC, Chr4: 158110496-158113496 |
| PHYH | Chr10: 13380252-13383252 | PHYH, Chr10: 13380252-13383252 |
| CCS | Chr11: 66115765-66118765 | CCS, Chr11: 66115765-66118765 |
| A26C1B | Chr2: 130593110-130596110 | A26C1B, Chr2: 130593110-130596110 |
| FRAT1 | Chr10: 99067511-99070511 | FRAT1, Chr10: 99067511-99070511 |
| PTPRN2 | Chr7: 158071743-158074743 | PTPRN2, Chr7: 158071743-158074743 |
| TST | Chr22: 35743937-35746937 | TST, Chr22: 35743937-35746937 |
| DFNB31 | Chr9: 116306051-116309051 | DFNB31, Chr9: 116306051-116309051 |
| PTCHD2 | Chr1: 11460381-11463381 | PTCHD2, Chr1: 11460381-11463381 |
| FBXO33 | Chr14: 38969871-38972871 | FBXO33, Chr14: 38969871-38972871 |
| HOXA5 | Chr7: 27148312-27151312 | HOXA5, Chr7: 27148312-27151312 |
| LOC751071 | Chr11: 62187854-62190854 | LOC751071, Chr11: 62187854-62190854 |
| NOM1 | Chr7: 156433677-156436677 | NOM1, Chr7: 156433677-156436677 |
| GPR26 | Chr10: 125414360-125417360 | GPR26, Chr10: 125414360-125417360 |
| PPP1R1B | Chr17: 35035204-35038204 | PPP1R1B, Chr17: 35035204-35038204 |
| MAP2K7 | Chr19: 7873264-7876264 | MAP2K7, Chr19: 7873264-7876264 |
| RELT | Chr11: 72763860-72766860 | RELT, Chr11: 72763860-72766860 |
| WDR65 | Chr1: 43409112-43412112 | WDR65, Chr1: 43409112-43412112 |
| CYB5R1 | Chr1: 201201527-201204527 | CYB5R1, Chr1: 201201527-201204527 |
| CCDC57 | Chr17: 77762478-77765478 | CCDC57, Chr17: 77762478-77765478 |
| USH1C | Chr11: 17521039-17524039 | USH1C, Chr11: 17521039-17524039 |
| SH3BGR | Chr21: 39738166-39741166 | SH3BGR, Chr21: 39738166-39741166 |
| RPL31 | Chr2: 100983622-100986622 | RPL31, Chr2: 100983622-100986622 |
| SLC27A6 | Chr5: 128327608-128330608 | SLC27A6, Chr5: 128327608-128330608 |
| SLC27A6 | Chr5: 128327611-128330611 | SLC27A6, Chr5: 128327611-128330611 |
| AURKAIP1 | Chr1: 1298925-1301925 | AURKAIP1, Chr1: 1298925-1301925 |
| AURKAIP1 | Chr1: 1298943-1301943 | AURKAIP1, Chr1: 1298943-1301943 |
| SCARF2 | Chr22: 19120646-19123646 | SCARF2, Chr22: 19120646-19123646 |
| C9orf25 | Chr9: 34447068-34450068 | C9orf25, Chr9: 34447068-34450068 |
| ULBP1 | Chr6: 150325335-150328335 | ULBP1, Chr6: 150325335-150328335 |
| CYB5A | Chr18: 70108701-70111701 | CYB5A, Chr18: 70108701-70111701 |
| MB | Chr22: 34342001-34345001 | MB, Chr22: 34342001-34345001 |
| ITPKA | Chr15: 39571913-39574913 | ITPKA, Chr15: 39571913-39574913 |
| DLK2 | Chr6: 43529764-43532764 | DLK2, Chr6: 43529764-43532764 |
| SLC35F3 | Chr1: 232105801-232108801 | SLC35F3, Chr1: 232105801-232108801 |
| CNTD1 | Chr17: 38202879-38205879 | CNTD1, Chr17: 38202879-38205879 |
| ZBTB12 | Chr6: 31976248-31979248 | ZBTB12, Chr6: 31976248-31979248 |
| DYNC1H1 | Chr14: 101499233-101502233 | DYNC1H1, Chr14: 101499233-101502233 |
| KCND2 | Chr7: 119699457-119702457 | KCND2, Chr7: 119699457-119702457 |
| FAM83H | Chr8: 144886402-144889402 | FAM83H, Chr8: 144886402-144889402 |
| UQCRC1 | Chr3: 48620602-48623602 | UQCRC1, Chr3: 48620602-48623602 |
| PROCA1 | Chr17: 24061499-24064499 | PROCA1, Chr17: 24061499-24064499 |
| PWP2 | Chr21: 44350135-44353135 | PWP2, Chr21: 44350135-44353135 |
| FSTL1 | Chr3: 121651015-121654015 | FSTL1, Chr3: 121651015-121654015 |
| HNT | Chr11: 131284421-131287421 | HNT, Chr11: 131284421-131287421 |
| KIFC3 | Chr16: 56392440-56395440 | KIFC3, Chr16: 56392440-56395440 |
| NTHL1 | Chr16: 2036368-2039368 | NTHL1, Chr16: 2036368-2039368 |
| DISP2 | Chr15: 38436225-38439225 | DISP2, Chr15: 38436225-38439225 |
| RPAP1 | Chr15: 39622256-39625256 | RPAP1, Chr15: 39622256-39625256 |
| EBNA1BP2 | Chr1: 43409018-43412018 | EBNA1BP2, Chr1: 43409018-43412018 |
| FBLN1 | Chr22: 44275882-44278882 | FBLN1, Chr22: 44275882-44278882 |
| GRASP | Chr12: 50685514-50688514 | GRASP, Chr12: 50685514-50688514 |
| ABCB6 | Chr2: 219790416-219793416 | ABCB6, Chr2: 219790416-219793416 |
| WDR45 | Chr23: 48843503-48846503 | WDR45, Chr23: 48843503-48846503 |
| FAM125B | Chr9: 128127448-128130448 | FAM125B, Chr9: 128127448-128130448 |
| EPHA8 | Chr1: 22761090-22764090 | EPHA8, Chr1: 22761090-22764090 |
| CST8 | Chr20: 23418265-23421265 | CST8, Chr20: 23418265-23421265 |
| SLIT1 | Chr10: 98934173-98937173 | SLIT1, Chr10: 98934173-98937173 |
| TSPAN17 | Chr5: 176005493-176008493 | TSPAN17, Chr5: 176005493-176008493 |
| FAM98A | Chr2: 33676366-33679366 | FAM98A, Chr2: 33676366-33679366 |
| C6orf201 | Chr6: 4022938-4025938 | C6orf201, Chr6: 4022938-4025938 |
| REXO1 | Chr19: 1797952-1800952 | REXO1, Chr19: 1797952-1800952 |
| NTRK2 | Chr9: 86471785-86474785 | NTRK2, Chr9: 86471785-86474785 |
| GSG1L | Chr16: 27980831-27983831 | GSG1L, Chr16: 27980831-27983831 |
| TCF15 | Chr20: 537410-540410 | TCF15, Chr20: 537410-540410 |
| DIRAS1 | Chr19: 2670869-2673869 | DIRAS1, Chr19: 2670869-2673869 |
| NTN1 | Chr17: 8864083-8867083 | NTN1, Chr17: 8864083-8867083 |
| RNF126 | Chr19: 612727-615727 | RNF126, Chr19: 612727-615727 |
| PHF19 | Chr9: 122677927-122680927 | PHF19, Chr9: 122677927-122680927 |
| RPS15 | Chr19: 1387862-1390862 | RPS15, Chr19: 1387862-1390862 |
| MARCKSL1 | Chr1: 32572910-32575910 | MARCKSL1, Chr1: 32572910-32575910 |
| C7orf41 | Chr7: 30139576-30142576 | C7orf41, Chr7: 30139576-30142576 |
| NEUROG1 | Chr5: 134898038-134901038 | NEUROG1, Chr5: 134898038-134901038 |
| SLC6A1 | Chr3: 11007919-11010919 | SLC6A1, Chr3: 11007919-11010919 |
| FAM19A5 | Chr22: 47262451-47265451 | FAM19A5, Chr22: 47262451-47265451 |
| ADAMTS17 | Chr15: 98698206-98701206 | ADAMTS17, Chr15: 98698206-98701206 |
| HES5 | Chr1: 2450044-2453044 | HES5, Chr1: 2450044-2453044 |
| MRPL43 | Chr10: 102735762-102738762 | MRPL43, Chr10: 102735762-102738762 |
| PLCD1 | Chr3: 38044637-38047637 | PLCD1, Chr3: 38044637-38047637 |
| C3orf64 | Chr3: 69143964-69146964 | C3orf64, Chr3: 69143964-69146964 |
| CHRNA3 | Chr15: 76698877-76701877 | CHRNA3, Chr15: 76698877-76701877 |
| RTBDN | Chr19: 12800707-12803707 | RTBDN, Chr19: 12800707-12803707 |
| C10orf88 | Chr10: 124702409-124705409 | C10orf88, Chr10: 124702409-124705409 |
| BMP3 | Chr4: 82169642-82172642 | BMP3, Chr4: 82169642-82172642 |
| FLJ10769 | Chr13: 110064508-110067508 | FLJ10769, Chr13: 110064508-110067508 |
| PLLP | Chr16: 55874572-55877572 | PLLP, Chr16: 55874572-55877572 |
| CD46 | Chr1: 205990524-205993524 | CD46, Chr1: 205990524-205993524 |
| ZNF331 | Chr19: 58714488-58717488 | ZNF331, Chr19: 58714488-58717488 |
| KATNAL1 | Chr13: 29778084-29781084 | KATNAL1, Chr13: 29778084-29781084 |
| ZDHHC18 | Chr1: 27024287-27027287 | ZDHHC18, Chr1: 27024287-27027287 |
| GRIN1 | Chr9: 139151929-139154929 | GRIN1, Chr9: 139151929-139154929 |
| MFSD4 | Chr1: 203803234-203806234 | MFSD4, Chr1: 203803234-203806234 |
| CNNM4 | Chr2: 96788865-96791865 | CNNM4, Chr2: 96788865-96791865 |
| LOC401399 | Chr7: 127787475-127790475 | LOC401399, Chr7: 127787475-127790475 |
| SLC6A6 | Chr3: 14417609-14420609 | SLC6A6, Chr3: 14417609-14420609 |
| C22orf32 | Chr22: 40804144-40807144 | C22orf32, Chr22: 40804144-40807144 |
| ALPL | Chr1: 21706944-21709944 | ALPL, Chr1: 21706944-21709944 |
| BSCL2 | Chr11: 62228863-62231863 | BSCL2, Chr11: 62228863-62231863 |
| SNCA | Chr4: 90975656-90978656 | SNCA, Chr4: 90975656-90978656 |
| PFKFB4 | Chr3: 48567731-48570731 | PFKFB4, Chr3: 48567731-48570731 |
| TMEM177 | Chr2: 120151712-120154712 | TMEM177, Chr2: 120151712-120154712 |
| C2orf70 | Chr2: 26637484-26640484 | C2orf70, Chr2: 26637484-26640484 |
| SLC46A2 | Chr9: 114691366-114694366 | SLC46A2, Chr9: 114691366-114694366 |
| PTMS | Chr12: 6744301-6747301 | PTMS, Chr12: 6744301-6747301 |
| AURKAIP1 | Chr1: 1299181-1302181 | AURKAIP1, Chr1: 1299181-1302181 |
| MKNK2 | Chr19: 2000743-2003743 | MKNK2, Chr19: 2000743-2003743 |
| T | Chr6: 166500621-166503621 | T, Chr6: 166500621-166503621 |
| B3GALT4 | Chr6: 33351394-33354394 | B3GALT4, Chr6: 33351394-33354394 |
| C12orf56 | Chr12: 63069112-63072112 | C12orf56, Chr12: 63069112-63072112 |
| ADAMTS10 | Chr19: 8580088-8583088 | ADAMTS10, Chr19: 8580088-8583088 |
| PMEPA1 | Chr20: 55716937-55719937 | PMEPA1, Chr20: 55716937-55719937 |
| SLC10A4 | Chr4: 48178705-48181705 | SLC10A4, Chr4: 48178705-48181705 |
| FGF4 | Chr11: 69297852-69300852 | FGF4, Chr11: 69297852-69300852 |
| ZNF518B | Chr4: 10066630-10069630 | ZNF518B, Chr4: 10066630-10069630 |
| PPAPDC1A | Chr10: 122204955-122207955 | PPAPDC1A, Chr10: 122204955-122207955 |
| CMTM3 | Chr16: 65194567-65197567 | CMTM3, Chr16: 65194567-65197567 |
| ANKMY1 | Chr2: 241144578-241147578 | ANKMY1, Chr2: 241144578-241147578 |
| UCN | Chr2: 27383134-27386134 | UCN, Chr2: 27383134-27386134 |
| CRHR2 | Chr7: 30687165-30690165 | CRHR2, Chr7: 30687165-30690165 |
| RELT | Chr11: 72763552-72766552 | RELT, Chr11: 72763552-72766552 |
| UCKL1 | Chr20: 62056712-62059712 | UCKL1, Chr20: 62056712-62059712 |
| MX1 | Chr21: 41718523-41721523 | MX1, Chr21: 41718523-41721523 |
| GOLGB1 | Chr3: 122949792-122952792 | GOLGB1, Chr3: 122949792-122952792 |
| SSPN | Chr12: 26238288-26241288 | SSPN, Chr12: 26238288-26241288 |
| FLNC | Chr7: 128256218-128259218 | FLNC, Chr7: 128256218-128259218 |
| EFS | Chr14: 22903182-22906182 | EFS, Chr14: 22903182-22906182 |
| RARRES1 | Chr3: 159931469-159934469 | RARRES1, Chr3: 159931469-159934469 |
| LRRC16B | Chr14: 23589545-23592545 | LRRC16B, Chr14: 23589545-23592545 |
| BCAM | Chr19: 50002677-50005677 | BCAM, Chr19: 50002677-50005677 |
| HOXA6 | Chr7: 27152393-27155393 | HOXA6, Chr7: 27152393-27155393 |
| PHYH | Chr10: 13380636-13383636 | PHYH, Chr10: 13380636-13383636 |
| SLC36A4 | Chr11: 92569243-92572243 | SLC36A4, Chr11: 92569243-92572243 |
| PRKAR1B | Chr7: 717159-720159 | PRKAR1B, Chr7: 717159-720159 |
| C9orf165 | Chr9: 34511537-34514537 | C9orf165, Chr9: 34511537-34514537 |
| MEGF6 | Chr1: 3516419-3519419 | MEGF6, Chr1: 3516419-3519419 |
| CAST | Chr5: 96062751-96065751 | CAST, Chr5: 96062751-96065751 |
| C6orf146 | Chr6: 4022890-4025890 | C6orf146, Chr6: 4022890-4025890 |
| WNT10A | Chr2: 219451998-219454998 | WNT10A, Chr2: 219451998-219454998 |
| VASN | Chr16: 4360349-4363349 | VASN, Chr16: 4360349-4363349 |
| CHD9 | Chr16: 51644945-51647945 | CHD9, Chr16: 51644945-51647945 |
| FAM109B | Chr22: 40798700-40801700 | FAM109B, Chr22: 40798700-40801700 |
| C6orf138 | Chr6: 48142884-48145884 | C6orf138, Chr6: 48142884-48145884 |
| TCL1A | Chr14: 95248786-95251786 | TCL1A, Chr14: 95248786-95251786 |
| TF | Chr3: 134946424-134949424 | TF, Chr3: 134946424-134949424 |
| PNMA3 | Chr23: 151973921-151976921 | PNMA3, Chr23: 151973921-151976921 |
| SMARCD3 | Chr7: 150603664-150606664 | SMARCD3, Chr7: 150603664-150606664 |
| COL9A2 | Chr1: 40554026-40557026 | COL9A2, Chr1: 40554026-40557026 |
| GPR135 | Chr14: 59000312-59003312 | GPR135, Chr14: 59000312-59003312 |
| CXCL16 | Chr17: 4588472-4591472 | CXCL16, Chr17: 4588472-4591472 |
| CACNA1A | Chr19: 13476774-13479774 | CACNA1A, Chr19: 13476774-13479774 |
| GFM2 | Chr5: 74097298-74100298 | GFM2, Chr5: 74097298-74100298 |
| ZNF786 | Chr7: 148417302-148420302 | ZNF786, Chr7: 148417302-148420302 |
| CCDC142 | Chr2: 74562131-74565131 | CCDC142, Chr2: 74562131-74565131 |
| MRC2 | Chr17: 58056993-58059993 | MRC2, Chr17: 58056993-58059993 |
| GRIN3A | Chr9: 103539183-103542183 | GRIN3A, Chr9: 103539183-103542183 |
| CHST14 | Chr15: 38549004-38552004 | CHST14, Chr15: 38549004-38552004 |
| KIRREL | Chr1: 156228186-156231186 | KIRREL, Chr1: 156228186-156231186 |
| CRABP1 | Chr15: 76418220-76421220 | CRABP1, Chr15: 76418220-76421220 |
| PHLDA3 | Chr1: 199703422-199706422 | PHLDA3, Chr1: 199703422-199706422 |
| BST1 | Chr4: 15312170-15315170 | BST1, Chr4: 15312170-15315170 |
| MCM6 | Chr2: 136348981-136351981 | MCM6, Chr2: 136348981-136351981 |
| PKP1 | Chr1: 199517702-199520702 | PKP1, Chr1: 199517702-199520702 |
| PLEKHG6 | Chr12: 6288362-6291362 | PLEKHG6, Chr12: 6288362-6291362 |
| JUP | Chr17: 37194975-37197975 | JUP, Chr17: 37194975-37197975 |
| NXNL1 | Chr19: 17431225-17434225 | NXNL1, Chr19: 17431225-17434225 |
| ZNF296 | Chr19: 50270028-50273028 | ZNF296, Chr19: 50270028-50273028 |
| CCDC87 | Chr11: 66115630-66118630 | CCDC87, Chr11: 66115630-66118630 |
| RASSF1 | Chr3: 50351871-50354871 | RASSF1, Chr3: 50351871-50354871 |
| LAS1L | Chr23: 64669892-64672892 | LAS1L, Chr23: 64669892-64672892 |
| NPHS2 | Chr1: 177810207-177813207 | NPHS2, Chr1: 177810207-177813207 |
| IFRD2 | Chr3: 50303423-50306423 | IFRD2, Chr3: 50303423-50306423 |
| RFTN1 | Chr3: 16528726-16531726 | RFTN1, Chr3: 16528726-16531726 |
| FNDC4 | Chr2: 27570090-27573090 | FNDC4, Chr2: 27570090-27573090 |
| NMUR1 | Chr2: 232101926-232104926 | NMUR1, Chr2: 232101926-232104926 |
| GFRA3 | Chr5: 137636652-137639652 | GFRA3, Chr5: 137636652-137639652 |
| P4HA3 | Chr11: 73698847-73701847 | P4HA3, Chr11: 73698847-73701847 |
| COL7A1 | Chr3: 48606097-48609097 | COL7A1, Chr3: 48606097-48609097 |
| DDX43 | Chr6: 74159691-74162691 | DDX43, Chr6: 74159691-74162691 |
| SLC38A3 | Chr3: 50216195-50219195 | SLC38A3, Chr3: 50216195-50219195 |
| LRRC43 | Chr12: 121232119-121235119 | LRRC43, Chr12: 121232119-121235119 |
| CKLF | Chr16: 65142466-65145466 | CKLF, Chr16: 65142466-65145466 |
| CKLF | Chr16: 65142472-65145472 | CKLF, Chr16: 65142472-65145472 |
| MCAM | Chr11: 118691550-118694550 | MCAM, Chr11: 118691550-118694550 |
| FLJ35773 | Chr17: 8641808-8644808 | FLJ35773, Chr17: 8641808-8644808 |
| CLEC14A | Chr14: 37793825-37796825 | CLEC14A, Chr14: 37793825-37796825 |
| ACRBP | Chr12: 6625341-6628341 | ACRBP, Chr12: 6625341-6628341 |
| PKDREJ | Chr22: 45036383-45039383 | PKDREJ, Chr22: 45036383-45039383 |
| KBTBD7 | Chr13: 40665202-40668202 | KBTBD7, Chr13: 40665202-40668202 |
| ETNK2 | Chr1: 202386430-202389430 | ETNK2, Chr1: 202386430-202389430 |
| LEPREL2 | Chr12: 6806332-6809332 | LEPREL2, Chr12: 6806332-6809332 |
| FAM162B | Chr6: 117192079-117195079 | FAM162B, Chr6: 117192079-117195079 |
| LYPLA1 | Chr8: 55175630-55178630 | LYPLA1, Chr8: 55175630-55178630 |
| CYP11A1 | Chr15: 72444106-72447106 | CYP11A1, Chr15: 72444106-72447106 |
| TGFBI | Chr5: 135390982-135393982 | TGFBI, Chr5: 135390982-135393982 |
| CD200 | Chr3: 113533105-113536105 | CD200, Chr3: 113533105-113536105 |
| ALDH1L1 | Chr3: 127380675-127383675 | ALDH1L1, Chr3: 127380675-127383675 |
| TUBA3E | Chr2: 130671004-130674004 | TUBA3E, Chr2: 130671004-130674004 |
| TMBIM1 | Chr2: 218864024-218867024 | TMBIM1, Chr2: 218864024-218867024 |
| TOP1MT | Chr8: 144486925-144489925 | TOP1MT, Chr8: 144486925-144489925 |
| DLK2 | Chr6: 43530264-43533264 | DLK2, Chr6: 43530264-43533264 |
| ALOX15 | Chr17: 4490209-4493209 | ALOX15, Chr17: 4490209-4493209 |
| COL1A1 | Chr17: 45632499-45635499 | COL1A1, Chr17: 45632499-45635499 |
| SFRP4 | Chr7: 37921550-37924550 | SFRP4, Chr7: 37921550-37924550 |
| AGER | Chr6: 32258501-32261501 | AGER, Chr6: 32258501-32261501 |
| ARSI | Chr5: 149661218-149664218 | ARSI, Chr5: 149661218-149664218 |
| RPS4Y2 | Chr24: 21325841-21328841 | RPS4Y2, Chr24: 21325841-21328841 |
| GGTLC1 | Chr20: 23915916-23918916 | GGTLC1, Chr20: 23915916-23918916 |
| HIST1H3I | Chr6: 27946578-27949578 | HIST1H3I, Chr6: 27946578-27949578 |
| CMTM3 | Chr16: 65194202-65197202 | CMTM3, Chr16: 65194202-65197202 |
| CMTM3 | Chr16: 65194213-65197213 | CMTM3, Chr16: 65194213-65197213 |
| ACTL7A | Chr9: 110662923-110665923 | ACTL7A, Chr9: 110662923-110665923 |
| PLA2R1 | Chr2: 160625867-160628867 | PLA2R1, Chr2: 160625867-160628867 |
| DCT | Chr13: 93928437-93931437 | DCT, Chr13: 93928437-93931437 |
| NRBP2 | Chr8: 144994688-144997688 | NRBP2, Chr8: 144994688-144997688 |
| C6orf154 | Chr6: 43584902-43587902 | C6orf154, Chr6: 43584902-43587902 |
| TCF21 | Chr6: 134250469-134253469 | TCF21, Chr6: 134250469-134253469 |
| TAPBP | Chr6: 33388467-33391467 | TAPBP, Chr6: 33388467-33391467 |
| TLR9 | Chr3: 52233719-52236719 | TLR9, Chr3: 52233719-52236719 |
| CES1 | Chr16: 54423076-54426076 | CES1, Chr16: 54423076-54426076 |
| CNNM1 | Chr10: 101077345-101080345 | CNNM1, Chr10: 101077345-101080345 |
| GNRH2 | Chr20: 2970767-2973767 | GNRH2, Chr20: 2970767-2973767 |
| KIAA1383 | Chr1: 231005760-231008760 | KIAA1383, Chr1: 231005760-231008760 |
| RIPK3 | Chr14: 23877582-23880582 | RIPK3, Chr14: 23877582-23880582 |
| CCDC19 | Chr1: 158135030-158138030 | CCDC19, Chr1: 158135030-158138030 |
| MC3R | Chr20: 54255694-54258694 | MC3R, Chr20: 54255694-54258694 |
| TPBG | Chr6: 83128757-83131757 | TPBG, Chr6: 83128757-83131757 |
| STAT5A | Chr17: 37691590-37694590 | STAT5A, Chr17: 37691590-37694590 |
| UPP1 | Chr7: 48093379-48096379 | UPP1, Chr7: 48093379-48096379 |
| SLC16A2 | Chr23: 73556309-73559309 | SLC16A2, Chr23: 73556309-73559309 |
| SUSD2 | Chr22: 22905943-22908943 | SUSD2, Chr22: 22905943-22908943 |
| CLDN5 | Chr22: 17891360-17894360 | CLDN5, Chr22: 17891360-17894360 |
| MYF6 | Chr12: 79624076-79627076 | MYF6, Chr12: 79624076-79627076 |
| FUT2 | Chr19: 53889539-53892539 | FUT2, Chr19: 53889539-53892539 |
| BSCL2 | Chr11: 62229946-62232946 | BSCL2, Chr11: 62229946-62232946 |
| 9-Sep | Chr17: 72956707-72959707 | 40795, Chr17: 72956707-72959707 |
| C20orf195 | Chr20: 61653316-61656316 | C20orf195, Chr20: 61653316-61656316 |
| GHRL | Chr3: 10305909-10308909 | GHRL, Chr3: 10305909-10308909 |
| AIM1L | Chr1: 26541530-26544530 | AIM1L, Chr1: 26541530-26544530 |
| ZNF217 | Chr20: 51631543-51634543 | ZNF217, Chr20: 51631543-51634543 |
| BCAS1 | Chr20: 52119211-52122211 | BCAS1, Chr20: 52119211-52122211 |
| TOMM7 | Chr7: 22827446-22830446 | TOMM7, Chr7: 22827446-22830446 |
| KLF1 | Chr19: 12857517-12860517 | KLF1, Chr19: 12857517-12860517 |
| LOC283331 | Chr12: 48483940-48486940 | LOC283331, Chr12: 48483940-48486940 |
| CYP11A1 | Chr15: 72445634-72448634 | CYP11A1, Chr15: 72445634-72448634 |
| TACC3 | Chr4: 1691563-1694563 | TACC3, Chr4: 1691563-1694563 |
| INPP5A | Chr10: 134199842-134202842 | INPP5A, Chr10: 134199842-134202842 |
| PSG11 | Chr19: 48220971-48223971 | PSG11, Chr19: 48220971-48223971 |
| THAP4 | Chr2: 242223828-242226828 | THAP4, Chr2: 242223828-242226828 |
| MRPL14 | Chr6: 44201669-44204669 | MRPL14, Chr6: 44201669-44204669 |
| CYB561 | Chr17: 58875777-58878777 | CYB561, Chr17: 58875777-58878777 |
| HTT | Chr4: 3044705-3047705 | HTT, Chr4: 3044705-3047705 |
| RPL7A | Chr9: 135203389-135206389 | RPL7A, Chr9: 135203389-135206389 |
| ZNF467 | Chr7: 149099728-149102728 | ZNF467, Chr7: 149099728-149102728 |
| CXXC5 | Chr5: 139006984-139009984 | CXXC5, Chr5: 139006984-139009984 |
| GPC1 | Chr2: 241022287-241025287 | GPC1, Chr2: 241022287-241025287 |
| VASH1 | Chr14: 76296487-76299487 | VASH1, Chr14: 76296487-76299487 |
| ABR | Chr17: 1028381-1031381 | ABR, Chr17: 1028381-1031381 |
| CELSR2 | Chr1: 109592663-109595663 | CELSR2, Chr1: 109592663-109595663 |
| SMPD4 | Chr2: 130654664-130657664 | SMPD4, Chr2: 130654664-130657664 |
| LGALS7 | Chr19: 43954487-43957487 | LGALS7, Chr19: 43954487-43957487 |
| PPP1R13B | Chr14: 103382180-103385180 | PPP1R13B, Chr14: 103382180-103385180 |
| TMCO4 | Chr1: 19997497-20000497 | TMCO4, Chr1: 19997497-20000497 |
| CYB561 | Chr17: 58875954-58878954 | CYB561, Chr17: 58875954-58878954 |
| PDE9A | Chr21: 42945430-42948430 | PDE9A, Chr21: 42945430-42948430 |
| 9-Sep | Chr17: 72787586-72790586 | 40795, Chr17: 72787586-72790586 |
| FLJ20433 | Chr9: 139436035-139439035 | FLJ20433, Chr9: 139436035-139439035 |
| LTBP3 | Chr11: 65080506-65083506 | LTBP3, Chr11: 65080506-65083506 |
| HSD11B2 | Chr16: 66021036-66024036 | HSD11B2, Chr16: 66021036-66024036 |
| TTLL9 | Chr20: 29920665-29923665 | TTLL9, Chr20: 29920665-29923665 |
| JAG2 | Chr14: 104704706-104707706 | JAG2, Chr14: 104704706-104707706 |
| TFAP2A | Chr6: 10521956-10524956 | TFAP2A, Chr6: 10521956-10524956 |
| RASD1 | Chr17: 17338932-17341932 | RASD1, Chr17: 17338932-17341932 |
| VEGFA | Chr6: 43844430-43847430 | VEGFA, Chr6: 43844430-43847430 |
| MARK3 | Chr14: 102919953-102922953 | MARK3, Chr14: 102919953-102922953 |
| C9orf86 | Chr9: 138820701-138823701 | C9orf86, Chr9: 138820701-138823701 |
| C19orf52 | Chr19: 10898923-10901923 | C19orf52, Chr19: 10898923-10901923 |
| ALKBH4 | Chr7: 101890793-101893793 | ALKBH4, Chr7: 101890793-101893793 |
| ARRB2 | Chr17: 4559037-4562037 | ARRB2, Chr17: 4559037-4562037 |
| ATP8B2 | Chr1: 152563159-152566159 | ATP8B2, Chr1: 152563159-152566159 |
| VDAC3 | Chr8: 42367046-42370046 | VDAC3, Chr8: 42367046-42370046 |
| RRP9 | Chr3: 51949462-51952462 | RRP9, Chr3: 51949462-51952462 |
| CCDC123 | Chr19: 38153209-38156209 | CCDC123, Chr19: 38153209-38156209 |
| PITX1 | Chr5: 134396363-134399363 | PITX1, Chr5: 134396363-134399363 |
| CLDN7 | Chr17: 7105019-7108019 | CLDN7, Chr17: 7105019-7108019 |
| OAZ1 | Chr19: 2219019-2222019 | OAZ1, Chr19: 2219019-2222019 |
| YTHDF1 | Chr20: 61316483-61319483 | YTHDF1, Chr20: 61316483-61319483 |
| HSP90AA1 | Chr14: 101621765-101624765 | HSP90AA1, Chr14: 101621765-101624765 |
| ATP10A | Chr15: 23657942-23660942 | ATP10A, Chr15: 23657942-23660942 |
| DGKD | Chr2: 233926391-233929391 | DGKD, Chr2: 233926391-233929391 |
| EEF1B2 | Chr2: 206731062-206734062 | EEF1B2, Chr2: 206731062-206734062 |
| RWDD2A | Chr6: 83958250-83961250 | RWDD2A, Chr6: 83958250-83961250 |
| LASS4 | Chr19: 8178756-8181756 | LASS4, Chr19: 8178756-8181756 |
| FBXO2 | Chr1: 11635826-11638826 | FBXO2, Chr1: 11635826-11638826 |
| C9orf58 | Chr9: 132960232-132963232 | C9orf58, Chr9: 132960232-132963232 |
| H1F0 | Chr22: 36529559-36532559 | H1F0, Chr22: 36529559-36532559 |
| PHOX2A | Chr11: 71631368-71634368 | PHOX2A, Chr11: 71631368-71634368 |
| C17orf76 | Chr17: 16334705-16337705 | C17orf76, Chr17: 16334705-16337705 |
| TSNARE1 | Chr8: 143480944-143483944 | TSNARE1, Chr8: 143480944-143483944 |
| ARFGAP2 | Chr11: 47153495-47156495 | ARFGAP2, Chr11: 47153495-47156495 |
| ESPN | Chr1: 6405934-6408934 | ESPN, Chr1: 6405934-6408934 |
| EPN1 | Chr19: 60876872-60879872 | EPN1, Chr19: 60876872-60879872 |
| HSPA9 | Chr5: 137937514-137940514 | HSPA9, Chr5: 137937514-137940514 |
| EFNB3 | Chr17: 7547744-7550744 | EFNB3, Chr17: 7547744-7550744 |
| SAMD14 | Chr17: 45560666-45563666 | SAMD14, Chr17: 45560666-45563666 |
| ACY1 | Chr3: 51991102-51994102 | ACY1, Chr3: 51991102-51994102 |
| LOC440957 | Chr3: 52544160-52547160 | LOC440957, Chr3: 52544160-52547160 |
| UFSP1 | Chr7: 100323775-100326775 | UFSP1, Chr7: 100323775-100326775 |
| LRP5 | Chr11: 67835183-67838183 | LRP5, Chr11: 67835183-67838183 |
| TPI1 | Chr12: 6845466-6848466 | TPI1, Chr12: 6845466-6848466 |
| AES | Chr19: 3011871-3014871 | AES, Chr19: 3011871-3014871 |
| CACNA2D2 | Chr3: 50514396-50517396 | CACNA2D2, Chr3: 50514396-50517396 |
| SBSN | Chr19: 40709593-40712593 | SBSN, Chr19: 40709593-40712593 |
| TRNT1 | Chr3: 3142099-3145099 | TRNT1, Chr3: 3142099-3145099 |
| HN1L | Chr16: 1666778-1669778 | HN1L, Chr16: 1666778-1669778 |
| ZNF326 | Chr1: 90231765-90234765 | ZNF326, Chr1: 90231765-90234765 |
| MRPL12 | Chr17: 77279311-77282311 | MRPL12, Chr17: 77279311-77282311 |
| FADS3 | Chr11: 61414082-61417082 | FADS3, Chr11: 61414082-61417082 |
| PDIA3 | Chr15: 41824381-41827381 | PDIA3, Chr15: 41824381-41827381 |
| MSTO1 | Chr1: 153845130-153848130 | MSTO1, Chr1: 153845130-153848130 |
| ZNF543 | Chr19: 62522188-62525188 | ZNF543, Chr19: 62522188-62525188 |
| ULBP3 | Chr6: 150430395-150433395 | ULBP3, Chr6: 150430395-150433395 |
| PLEKHA2 | Chr8: 38876409-38879409 | PLEKHA2, Chr8: 38876409-38879409 |
| AQP11 | Chr11: 76976827-76979827 | AQP11, Chr11: 76976827-76979827 |
| PLAA | Chr9: 26935639-26938639 | PLAA, Chr9: 26935639-26938639 |
| ZBTB48 | Chr1: 6561197-6564197 | ZBTB48, Chr1: 6561197-6564197 |
| NDUFS8 | Chr11: 67553169-67556169 | NDUFS8, Chr11: 67553169-67556169 |
| SGK2 | Chr20: 41619599-41622599 | SGK2, Chr20: 41619599-41622599 |
| VPS18 | Chr15: 38972419-38975419 | VPS18, Chr15: 38972419-38975419 |
| RUFY1 | Chr5: 178908676-178911676 | RUFY1, Chr5: 178908676-178911676 |
| MBP | Chr18: 72972262-72975262 | MBP, Chr18: 72972262-72975262 |
| ACD | Chr16: 66250719-66253719 | ACD, Chr16: 66250719-66253719 |
| PRR17 | Chr20: 62138436-62141436 | PRR17, Chr20: 62138436-62141436 |
| ACTB | Chr7: 5535258-5538258 | ACTB, Chr7: 5535258-5538258 |
| TRAP1 | Chr16: 3706099-3709099 | TRAP1, Chr16: 3706099-3709099 |
| FBXO44 | Chr1: 11635518-11638518 | FBXO44, Chr1: 11635518-11638518 |
| CALCB | Chr11: 15050221-15053221 | CALCB, Chr11: 15050221-15053221 |
| SMURF1 | Chr7: 98578159-98581159 | SMURF1, Chr7: 98578159-98581159 |
| FADD | Chr11: 69725416-69728416 | FADD, Chr11: 69725416-69728416 |
| ZNF142 | Chr2: 219231005-219234005 | ZNF142, Chr2: 219231005-219234005 |
| C7orf43 | Chr7: 99592738-99595738 | C7orf43, Chr7: 99592738-99595738 |
| C9orf103 | Chr9: 85426285-85429285 | C9orf103, Chr9: 85426285-85429285 |
| TMEM109 | Chr11: 60436752-60439752 | TMEM109, Chr11: 60436752-60439752 |
| CHMP2A | Chr19: 63756667-63759667 | CHMP2A, Chr19: 63756667-63759667 |
| KCTD21 | Chr11: 77575812-77578812 | KCTD21, Chr11: 77575812-77578812 |
| RNF215 | Chr22: 29111802-29114802 | RNF215, Chr22: 29111802-29114802 |
| ZNF70 | Chr22: 22421779-22424779 | ZNF70, Chr22: 22421779-22424779 |
| FAM65A | Chr16: 66118754-66121754 | FAM65A, Chr16: 66118754-66121754 |
| MOSC1 | Chr1: 219025393-219028393 | MOSC1, Chr1: 219025393-219028393 |
| COMT | Chr22: 18307808-18310808 | COMT, Chr22: 18307808-18310808 |
| COPG | Chr3: 130449642-130452642 | COPG, Chr3: 130449642-130452642 |
| CDC25B | Chr20: 3722900-3725900 | CDC25B, Chr20: 3722900-3725900 |
| STMN3 | Chr20: 61753724-61756724 | STMN3, Chr20: 61753724-61756724 |
| KPNA3 | Chr13: 49263558-49266558 | KPNA3, Chr13: 49263558-49266558 |
| RNF130 | Chr5: 179430215-179433215 | RNF130, Chr5: 179430215-179433215 |
| CSNK1D | Chr17: 77823362-77826362 | CSNK1D, Chr17: 77823362-77826362 |
| ATP2A1 | Chr16: 28795809-28798809 | ATP2A1, Chr16: 28795809-28798809 |
| RGS11 | Chr16: 264415-267415 | RGS11, Chr16: 264415-267415 |
| PLEKHG3 | Chr14: 64239445-64242445 | PLEKHG3, Chr14: 64239445-64242445 |
| TRIM11 | Chr1: 226659640-226662640 | TRIM11, Chr1: 226659640-226662640 |
| BID | Chr22: 16635758-16638758 | BID, Chr22: 16635758-16638758 |
| KIAA0562 | Chr1: 3762157-3765157 | KIAA0562, Chr1: 3762157-3765157 |
| ZNF142 | Chr2: 219231099-219234099 | ZNF142, Chr2: 219231099-219234099 |
| QSOX2 | Chr9: 138276008-138279008 | QSOX2, Chr9: 138276008-138279008 |
| RAMP1 | Chr2: 238431425-238434425 | RAMP1, Chr2: 238431425-238434425 |
| RPL5 | Chr1: 93068681-93071681 | RPL5, Chr1: 93068681-93071681 |
| GLRX3 | Chr10: 131823152-131826152 | GLRX3, Chr10: 131823152-131826152 |
| HLTF | Chr3: 150285531-150288531 | HLTF, Chr3: 150285531-150288531 |
| TMEM132A | Chr11: 60446988-60449988 | TMEM132A, Chr11: 60446988-60449988 |
| PNPLA5 | Chr22: 42617690-42620690 | PNPLA5, Chr22: 42617690-42620690 |
| NFX1 | Chr9: 33279009-33282009 | NFX1, Chr9: 33279009-33282009 |
| C22orf36 | Chr22: 23317535-23320535 | C22orf36, Chr22: 23317535-23320535 |
| SYNGR3 | Chr16: 1978468-1981468 | SYNGR3, Chr16: 1978468-1981468 |
| GLRX2 | Chr1: 191339679-191342679 | GLRX2, Chr1: 191339679-191342679 |
| MCART1 | Chr9: 37892850-37895850 | MCART1, Chr9: 37892850-37895850 |
| PAQR5 | Chr15: 67376847-67379847 | PAQR5, Chr15: 67376847-67379847 |
| BRMS1 | Chr11: 65867658-65870658 | BRMS1, Chr11: 65867658-65870658 |
| ZNF30 | Chr19: 40108146-40111146 | ZNF30, Chr19: 40108146-40111146 |
| CHIC1 | Chr23: 72698208-72701208 | CHIC1, Chr23: 72698208-72701208 |
| ATXN7L2 | Chr1: 109826583-109829583 | ATXN7L2, Chr1: 109826583-109829583 |
| HYPK | Chr15: 41878412-41881412 | HYPK, Chr15: 41878412-41881412 |
| RHOD | Chr11: 66579396-66582396 | RHOD, Chr11: 66579396-66582396 |
| PPP2R5B | Chr11: 64447255-64450255 | PPP2R5B, Chr11: 64447255-64450255 |
| HPCAL4 | Chr1: 39928176-39931176 | HPCAL4, Chr1: 39928176-39931176 |
| SAPS2 | Chr22: 49127125-49130125 | SAPS2, Chr22: 49127125-49130125 |
| SYT5 | Chr19: 60382032-60385032 | SYT5, Chr19: 60382032-60385032 |
| PPP2R2C | Chr4: 6523727-6526727 | PPP2R2C, Chr4: 6523727-6526727 |
| TIMM13 | Chr19: 2377375-2380375 | TIMM13, Chr19: 2377375-2380375 |
| PAPLN | Chr14: 72772457-72775457 | PAPLN, Chr14: 72772457-72775457 |
| HCCA2 | Chr11: 1740577-1743577 | HCCA2, Chr11: 1740577-1743577 |
| TRIM17 | Chr1: 226669477-226672477 | TRIM17, Chr1: 226669477-226672477 |
| RHBDF1 | Chr16: 61129-64129 | RHBDF1, Chr16: 61129-64129 |
| RAVER1 | Chr19: 10303814-10306814 | RAVER1, Chr19: 10303814-10306814 |
| SBNO2 | Chr19: 1123782-1126782 | SBNO2, Chr19: 1123782-1126782 |
| INF2 | Chr14: 104225487-104228487 | INF2, Chr14: 104225487-104228487 |
| CRTC2 | Chr1: 152196167-152199167 | CRTC2, Chr1: 152196167-152199167 |
| SHH | Chr7: 155296228-155299228 | SHH, Chr7: 155296228-155299228 |
| C14orf151 | Chr14: 104225502-104228502 | C14orf151, Chr14: 104225502-104228502 |
| LRRC34 | Chr3: 171011506-171014506 | LRRC34, Chr3: 171011506-171014506 |
| C1orf2 | Chr1: 153490398-153493398 | C1orf2, Chr1: 153490398-153493398 |
| CHMP2A | Chr19: 63756798-63759798 | CHMP2A, Chr19: 63756798-63759798 |
| BDP1 | Chr5: 70785697-70788697 | BDP1, Chr5: 70785697-70788697 |
| FLOT2 | Chr17: 24247341-24250341 | FLOT2, Chr17: 24247341-24250341 |
| FDX1L | Chr19: 10286191-10289191 | FDX1L, Chr19: 10286191-10289191 |
| MS4A12 | Chr11: 60015326-60018326 | MS4A12, Chr11: 60015326-60018326 |
| C20orf185 | Chr20: 31105390-31108390 | C20orf185, Chr20: 31105390-31108390 |
| NPAL1 | Chr4: 47712047-47715047 | NPAL1, Chr4: 47712047-47715047 |
| C9orf50 | Chr9: 131421376-131424376 | C9orf50, Chr9: 131421376-131424376 |
| EXOC6B | Chr2: 72905185-72908185 | EXOC6B, Chr2: 72905185-72908185 |
| CXXC1 | Chr18: 46067190-46070190 | CXXC1, Chr18: 46067190-46070190 |
| AES | Chr19: 3012464-3015464 | AES, Chr19: 3012464-3015464 |
| CPXM2 | Chr10: 125639990-125642990 | CPXM2, Chr10: 125639990-125642990 |
| ALDOA | Chr16: 29981973-29984973 | ALDOA, Chr16: 29981973-29984973 |
| TCEA2 | Chr20: 62162953-62165953 | TCEA2, Chr20: 62162953-62165953 |
| C6orf195 | Chr6: 2578797-2581797 | C6orf195, Chr6: 2578797-2581797 |
| FTL | Chr19: 54158877-54161877 | FTL, Chr19: 54158877-54161877 |
| LOXL1 | Chr15: 72004341-72007341 | LOXL1, Chr15: 72004341-72007341 |
| HIVEP3 | Chr1: 42155583-42158583 | HIVEP3, Chr1: 42155583-42158583 |
| NLRP6 | Chr11: 267069-270069 | NLRP6, Chr11: 267069-270069 |
| GLRX2 | Chr1: 191340367-191343367 | GLRX2, Chr1: 191340367-191343367 |
| ALDOA | Chr16: 29981600-29984600 | ALDOA, Chr16: 29981600-29984600 |
| DMPK | Chr19: 50976155-50979155 | DMPK, Chr19: 50976155-50979155 |
| SRA1 | Chr5: 139916362-139919362 | SRA1, Chr5: 139916362-139919362 |
| KIF12 | Chr9: 115899658-115902658 | KIF12, Chr9: 115899658-115902658 |
| SRC | Chr20: 35405001-35408001 | SRC, Chr20: 35405001-35408001 |
| GEFT | Chr12: 56288729-56291729 | GEFT, Chr12: 56288729-56291729 |
| C20orf144 | Chr20: 31712280-31715280 | C20orf144, Chr20: 31712280-31715280 |
| RASIP1 | Chr19: 53934282-53937282 | RASIP1, Chr19: 53934282-53937282 |
| TREH | Chr11: 118054091-118057091 | TREH, Chr11: 118054091-118057091 |
| TNXB | Chr6: 32183629-32186629 | TNXB, Chr6: 32183629-32186629 |
| REXO1L1 | Chr8: 86761478-86764478 | REXO1L1, Chr8: 86761478-86764478 |
| CA9 | Chr9: 35662414-35665414 | CA9, Chr9: 35662414-35665414 |
| DYSFIP1 | Chr17: 77384715-77387715 | DYSFIP1, Chr17: 77384715-77387715 |
| FBP2 | Chr9: 96394396-96397396 | FBP2, Chr9: 96394396-96397396 |
| CYP2A13 | Chr19: 46284707-46287707 | CYP2A13, Chr19: 46284707-46287707 |
| MPO | Chr17: 53711795-53714795 | MPO, Chr17: 53711795-53714795 |
| APOA1 | Chr11: 116212048-116215048 | APOA1, Chr11: 116212048-116215048 |
| MMP9 | Chr20: 44069453-44072453 | MMP9, Chr20: 44069453-44072453 |
| NGEF | Chr2: 233584695-233587695 | NGEF, Chr2: 233584695-233587695 |
| SPSB3 | Chr16: 1771082-1774082 | SPSB3, Chr16: 1771082-1774082 |
| ATAD3A | Chr1: 1435917-1438917 | ATAD3A, Chr1: 1435917-1438917 |
| RNF212 | Chr4: 1095850-1098850 | RNF212, Chr4: 1095850-1098850 |
| HUNK | Chr21: 32165998-32168998 | HUNK, Chr21: 32165998-32168998 |
| MCRS1 | Chr12: 48246678-48249678 | MCRS1, Chr12: 48246678-48249678 |
| MAPK11 | Chr22: 49049406-49052406 | MAPK11, Chr22: 49049406-49052406 |
| MEPCE | Chr7: 99863964-99866964 | MEPCE, Chr7: 99863964-99866964 |
| C9orf116 | Chr9: 137530082-137533082 | C9orf116, Chr9: 137530082-137533082 |
| ESRRA | Chr11: 63828119-63831119 | ESRRA, Chr11: 63828119-63831119 |
| PQLC3 | Chr2: 11211490-11214490 | PQLC3, Chr2: 11211490-11214490 |
| FAM128B | Chr2: 130654672-130657672 | FAM128B, Chr2: 130654672-130657672 |
| PARD6A | Chr16: 66250851-66253851 | PARD6A, Chr16: 66250851-66253851 |
| KCNH3 | Chr12: 48217706-48220706 | KCNH3, Chr12: 48217706-48220706 |
| SLC7A10 | Chr19: 38407048-38410048 | SLC7A10, Chr19: 38407048-38410048 |
| PCNXL3 | Chr11: 65138858-65141858 | PCNXL3, Chr11: 65138858-65141858 |
| OR2C1 | Chr16: 3344389-3347389 | OR2C1, Chr16: 3344389-3347389 |
| CNP | Chr17: 37370784-37373784 | CNP, Chr17: 37370784-37373784 |
| SCRT1 | Chr8: 145529251-145532251 | SCRT1, Chr8: 145529251-145532251 |
| LOC645191 | Chr19: 2257656-2260656 | LOC645191, Chr19: 2257656-2260656 |
| ZBTB45 | Chr19: 63721233-63724233 | ZBTB45, Chr19: 63721233-63724233 |
| RARG | Chr12: 51898917-51901917 | RARG, Chr12: 51898917-51901917 |
| VENTX | Chr10: 134899897-134902897 | VENTX, Chr10: 134899897-134902897 |
| RGS10 | Chr10: 121284535-121287535 | RGS10, Chr10: 121284535-121287535 |
| CYP4F22 | Chr19: 15478835-15481835 | CYP4F22, Chr19: 15478835-15481835 |
| MYO1G | Chr7: 44983693-44986693 | MYO1G, Chr7: 44983693-44986693 |
| CCDC42 | Chr17: 8587379-8590379 | CCDC42, Chr17: 8587379-8590379 |
| NRSN2 | Chr20: 276392-279392 | NRSN2, Chr20: 276392-279392 |
| TCF3 | Chr19: 1599777-1602777 | TCF3, Chr19: 1599777-1602777 |
| CHRNB4 | Chr15: 76719142-76722142 | CHRNB4, Chr15: 76719142-76722142 |
| MCRS1 | Chr12: 48244989-48247989 | MCRS1, Chr12: 48244989-48247989 |
| LPPR2 | Chr19: 11325606-11328606 | LPPR2, Chr19: 11325606-11328606 |
| TNNC1 | Chr3: 52461598-52464598 | TNNC1, Chr3: 52461598-52464598 |
| FBXL19 | Chr16: 30841896-30844896 | FBXL19, Chr16: 30841896-30844896 |
| ATAD3C | Chr1: 1373431-1376431 | ATAD3C, Chr1: 1373431-1376431 |
| GPR32 | Chr19: 55964169-55967169 | GPR32, Chr19: 55964169-55967169 |
| VTN | Chr17: 23720000-23723000 | VTN, Chr17: 23720000-23723000 |
| TOB1 | Chr17: 46294912-46297912 | TOB1, Chr17: 46294912-46297912 |
| 10-Mar | Chr17: 58236243-58239243 | 40612, Chr17: 58236243-58239243 |
| NHSL2 | Chr23: 71268723-71271723 | NHSL2, Chr23: 71268723-71271723 |
| NAALADL1 | Chr11: 64581085-64584085 | NAALADL1, Chr11: 64581085-64584085 |
| LRG1 | Chr19: 4489536-4492536 | LRG1, Chr19: 4489536-4492536 |
| SH2D4B | Chr10: 82286137-82289137 | SH2D4B, Chr10: 82286137-82289137 |
| CHIT1 | Chr1: 201463983-201466983 | CHIT1, Chr1: 201463983-201466983 |
| TJP3 | Chr19: 3677873-3680873 | TJP3, Chr19: 3677873-3680873 |
| HDHD3 | Chr9: 115176662-115179662 | HDHD3, Chr9: 115176662-115179662 |
| NUDT1 | Chr7: 2247565-2250565 | NUDT1, Chr7: 2247565-2250565 |
| MAN1C1 | Chr1: 25815045-25818045 | MAN1C1, Chr1: 25815045-25818045 |
| DUSP8 | Chr11: 1548226-1551226 | DUSP8, Chr11: 1548226-1551226 |
| GPR12 | Chr13: 26230464-26233464 | GPR12, Chr13: 26230464-26233464 |
| FAM54B | Chr1: 26018405-26021405 | FAM54B, Chr1: 26018405-26021405 |
| ANKRD24 | Chr19: 4132850-4135850 | ANKRD24, Chr19: 4132850-4135850 |
| PHC2 | Chr1: 33612281-33615281 | PHC2, Chr1: 33612281-33615281 |
| XRCC3 | Chr14: 103250076-103253076 | XRCC3, Chr14: 103250076-103253076 |
| FBXL14 | Chr12: 1572092-1575092 | FBXL14, Chr12: 1572092-1575092 |
| PCTK1 | Chr23: 46961558-46964558 | PCTK1, Chr23: 46961558-46964558 |
| ZNF524 | Chr19: 60802041-60805041 | ZNF524, Chr19: 60802041-60805041 |
| KHK | Chr2: 27161614-27164614 | KHK, Chr2: 27161614-27164614 |
| GJB3 | Chr1: 35018997-35021997 | GJB3, Chr1: 35018997-35021997 |
| ACP5 | Chr19: 11549092-11552092 | ACP5, Chr19: 11549092-11552092 |
| BAT4 | Chr6: 31739642-31742642 | BAT4, Chr6: 31739642-31742642 |
| CIC | Chr19: 47479156-47482156 | CIC, Chr19: 47479156-47482156 |
| GPC2 | Chr7: 99611426-99614426 | GPC2, Chr7: 99611426-99614426 |
| TRIM46 | Chr1: 153411483-153414483 | TRIM46, Chr1: 153411483-153414483 |
| CHCHD10 | Chr22: 22438641-22441641 | CHCHD10, Chr22: 22438641-22441641 |
| E4F1 | Chr16: 2212067-2215067 | E4F1, Chr16: 2212067-2215067 |
| YIPF2 | Chr19: 10898857-10901857 | YIPF2, Chr19: 10898857-10901857 |
| SEMA3F | Chr3: 50166351-50169351 | SEMA3F, Chr3: 50166351-50169351 |
| TBL3 | Chr16: 1960564-1963564 | TBL3, Chr16: 1960564-1963564 |
| KLHL26 | Chr19: 18607337-18610337 | KLHL26, Chr19: 18607337-18610337 |
| DUSP15 | Chr20: 29919962-29922962 | DUSP15, Chr20: 29919962-29922962 |
| ACP5 | Chr19: 11549301-11552301 | ACP5, Chr19: 11549301-11552301 |
| DAAM2 | Chr6: 39867270-39870270 | DAAM2, Chr6: 39867270-39870270 |
| RPL36 | Chr19: 5639845-5642845 | RPL36, Chr19: 5639845-5642845 |
| SLC6A8 | Chr23: 152605445-152608445 | SLC6A8, Chr23: 152605445-152608445 |
| NOLA2 | Chr5: 177512067-177515067 | NOLA2, Chr5: 177512067-177515067 |
| NDUFS1 | Chr2: 206730932-206733932 | NDUFS1, Chr2: 206730932-206733932 |
| PGM3 | Chr6: 83958154-83961154 | PGM3, Chr6: 83958154-83961154 |
| FBXO44 | Chr1: 11636000-11639000 | FBXO44, Chr1: 11636000-11639000 |
| DFFB | Chr1: 3762204-3765204 | DFFB, Chr1: 3762204-3765204 |
| SLC9A1 | Chr1: 27352488-27355488 | SLC9A1, Chr1: 27352488-27355488 |
| SERINC4 | Chr15: 41878047-41881047 | SERINC4, Chr15: 41878047-41881047 |
| KIAA1949 | Chr6: 30760463-30763463 | KIAA1949, Chr6: 30760463-30763463 |
| TMEM40 | Chr3: 12774308-12777308 | TMEM40, Chr3: 12774308-12777308 |
| TMEM129 | Chr4: 1691382-1694382 | TMEM129, Chr4: 1691382-1694382 |
| TBC1D8 | Chr2: 101132778-101135778 | TBC1D8, Chr2: 101132778-101135778 |
| IDI1 | Chr10: 1083561-1086561 | IDI1, Chr10: 1083561-1086561 |
| TTLL12 | Chr22: 41911551-41914551 | TTLL12, Chr22: 41911551-41914551 |
| SURF2 | Chr9: 135211748-135214748 | SURF2, Chr9: 135211748-135214748 |
| CBX2 | Chr17: 75365087-75368087 | CBX2, Chr17: 75365087-75368087 |
| ANKRD13B | Chr17: 24943152-24946152 | ANKRD13B, Chr17: 24943152-24946152 |
| AZI1 | Chr17: 76809846-76812846 | AZI1, Chr17: 76809846-76812846 |
| SLC12A9 | Chr7: 100286793-100289793 | SLC12A9, Chr7: 100286793-100289793 |
| ZNF362 | Chr1: 33493260-33496260 | ZNF362, Chr1: 33493260-33496260 |
| DPP3 | Chr11: 66002955-66005955 | DPP3, Chr11: 66002955-66005955 |
| EPHB6 | Chr7: 142261413-142264413 | EPHB6, Chr7: 142261413-142264413 |
| RELA | Chr11: 65185451-65188451 | RELA, Chr11: 65185451-65188451 |
| DUSP15 | Chr20: 29920172-29923172 | DUSP15, Chr20: 29920172-29923172 |
| LOC196993 | Chr15: 69193393-69196393 | LOC196993, Chr15: 69193393-69196393 |
| RLTPR | Chr16: 66235030-66238030 | RLTPR, Chr16: 66235030-66238030 |
| PARP3 | Chr3: 51949900-51952900 | PARP3, Chr3: 51949900-51952900 |
| BCS1L | Chr2: 219231122-219234122 | BCS1L, Chr2: 219231122-219234122 |
| DNMT3A | Chr2: 25327184-25330184 | DNMT3A, Chr2: 25327184-25330184 |
| USP35 | Chr11: 77576105-77579105 | USP35, Chr11: 77576105-77579105 |
| SH3BP2 | Chr4: 2788838-2791838 | SH3BP2, Chr4: 2788838-2791838 |
| USHBP1 | Chr19: 17235044-17238044 | USHBP1, Chr19: 17235044-17238044 |
| SNN | Chr16: 11668301-11671301 | SNN, Chr16: 11668301-11671301 |
| PCTK1 | Chr23: 46961075-46964075 | PCTK1, Chr23: 46961075-46964075 |
| MED22 | Chr9: 135203293-135206293 | MED22, Chr9: 135203293-135206293 |
| NFKBIL2 | Chr8: 145639120-145642120 | NFKBIL2, Chr8: 145639120-145642120 |
| POP7 | Chr7: 100140111-100143111 | POP7, Chr7: 100140111-100143111 |
| TRAF7 | Chr16: 2144299-2147299 | TRAF7, Chr16: 2144299-2147299 |
| MICALL1 | Chr22: 36630791-36633791 | MICALL1, Chr22: 36630791-36633791 |
| RPS21 | Chr20: 60394015-60397015 | RPS21, Chr20: 60394015-60397015 |
| CORO1A | Chr16: 30100926-30103926 | CORO1A, Chr16: 30100926-30103926 |
| CARD10 | Chr22: 36243656-36246656 | CARD10, Chr22: 36243656-36246656 |
| P2RX2 | Chr12: 131703975-131706975 | P2RX2, Chr12: 131703975-131706975 |
| POLD2 | Chr7: 44128172-44131172 | POLD2, Chr7: 44128172-44131172 |
| DGCR8 | Chr22: 18446333-18449333 | DGCR8, Chr22: 18446333-18449333 |
| IQSEC1 | Chr3: 12982460-12985460 | IQSEC1, Chr3: 12982460-12985460 |
| FAM54B | Chr1: 26017770-26020770 | FAM54B, Chr1: 26017770-26020770 |
| EPO | Chr7: 100154858-100157858 | EPO, Chr7: 100154858-100157858 |
| ITGB4 | Chr17: 71227610-71230610 | ITGB4, Chr17: 71227610-71230610 |
| ELFN2 | Chr22: 36151951-36154951 | ELFN2, Chr22: 36151951-36154951 |
| LRRC16A | Chr6: 25386126-25389126 | LRRC16A, Chr6: 25386126-25389126 |
| LOC90379 | Chr19: 13922818-13925818 | LOC90379, Chr19: 13922818-13925818 |
| RAB37 | Chr17: 70243455-70246455 | RAB37, Chr17: 70243455-70246455 |
| ADRA1B | Chr5: 159274817-159277817 | ADRA1B, Chr5: 159274817-159277817 |
| B3GNT1 | Chr11: 65870237-65873237 | B3GNT1, Chr11: 65870237-65873237 |
| SRM | Chr1: 11041178-11044178 | SRM, Chr1: 11041178-11044178 |
| ABHD11 | Chr7: 72789620-72792620 | ABHD11, Chr7: 72789620-72792620 |
| SPECC1 | Chr17: 19998493-20001493 | SPECC1, Chr17: 19998493-20001493 |
| GMPPB | Chr3: 49734888-49737888 | GMPPB, Chr3: 49734888-49737888 |
| DUSP23 | Chr1: 158015882-158018882 | DUSP23, Chr1: 158015882-158018882 |
| ARRDC2 | Chr19: 17971443-17974443 | ARRDC2, Chr19: 17971443-17974443 |
| ARFGAP1 | Chr20: 61373109-61376109 | ARFGAP1, Chr20: 61373109-61376109 |
| SPIRE2 | Chr16: 88420907-88423907 | SPIRE2, Chr16: 88420907-88423907 |
| ZBTB24 | Chr6: 109909633-109912633 | ZBTB24, Chr6: 109909633-109912633 |
| MACROD1 | Chr11: 63688594-63691594 | MACROD1, Chr11: 63688594-63691594 |
| CHCHD5 | Chr2: 113057006-113060006 | CHCHD5, Chr2: 113057006-113060006 |
| PYCRL | Chr8: 144761387-144764387 | PYCRL, Chr8: 144761387-144764387 |
| TMEM121 | Chr14: 105062497-105065497 | TMEM121, Chr14: 105062497-105065497 |
| IFT74 | Chr9: 26935809-26938809 | IFT74, Chr9: 26935809-26938809 |
| MAPKAPK3 | Chr3: 50628104-50631104 | MAPKAPK3, Chr3: 50628104-50631104 |
| FAM125A | Chr19: 17390411-17393411 | FAM125A, Chr19: 17390411-17393411 |
| LOXL4 | Chr10: 100016497-100019497 | LOXL4, Chr10: 100016497-100019497 |
| ZNF517 | Chr8: 145993564-145996564 | ZNF517, Chr8: 145993564-145996564 |
| HDAC7 | Chr12: 46498530-46501530 | HDAC7, Chr12: 46498530-46501530 |
| CNOT3 | Chr19: 59331760-59334760 | CNOT3, Chr19: 59331760-59334760 |
| TMEM179 | Chr14: 104140642-104143642 | TMEM179, Chr14: 104140642-104143642 |
| CDK2AP2 | Chr11: 67031178-67034178 | CDK2AP2, Chr11: 67031178-67034178 |
| DGKZ | Chr11: 46324031-46327031 | DGKZ, Chr11: 46324031-46327031 |
| RALGDS | Chr9: 134984882-134987882 | RALGDS, Chr9: 134984882-134987882 |
| CTSD | Chr11: 1740298-1743298 | CTSD, Chr11: 1740298-1743298 |
| TMEM219 | Chr16: 29879351-29882351 | TMEM219, Chr16: 29879351-29882351 |
| KCTD17 | Chr22: 35776224-35779224 | KCTD17, Chr22: 35776224-35779224 |
| LGI2 | Chr4: 24639913-24642913 | LGI2, Chr4: 24639913-24642913 |
| GSTP1 | Chr11: 67106361-67109361 | GSTP1, Chr11: 67106361-67109361 |
| PPAN-P2RY11 | Chr19: 10076570-10079570 | PPAN-P2RY11, Chr19: 10076570-10079570 |
| PPL | Chr16: 4925637-4928637 | PPL, Chr16: 4925637-4928637 |
| C19orf40 | Chr19: 38153487-38156487 | C19orf40, Chr19: 38153487-38156487 |
| KCNH6 | Chr17: 58952926-58955926 | KCNH6, Chr17: 58952926-58955926 |
| TEKT2 | Chr1: 36320762-36323762 | TEKT2, Chr1: 36320762-36323762 |
| SYDE1 | Chr19: 15077713-15080713 | SYDE1, Chr19: 15077713-15080713 |
| LTBR | Chr12: 6362117-6365117 | LTBR, Chr12: 6362117-6365117 |
| RASL11A | Chr13: 26740963-26743963 | RASL11A, Chr13: 26740963-26743963 |
| ZNF575 | Chr19: 48727668-48730668 | ZNF575, Chr19: 48727668-48730668 |
| CHST1 | Chr11: 45642248-45645248 | CHST1, Chr11: 45642248-45645248 |
| PRRT2 | Chr16: 29729409-29732409 | PRRT2, Chr16: 29729409-29732409 |
| CHMP6 | Chr17: 76578735-76581735 | CHMP6, Chr17: 76578735-76581735 |
| ISCA1 | Chr9: 88085810-88088810 | ISCA1, Chr9: 88085810-88088810 |
| CCNYL1 | Chr2: 208283008-208286008 | CCNYL1, Chr2: 208283008-208286008 |
| ITGA3 | Chr17: 45486838-45489838 | ITGA3, Chr17: 45486838-45489838 |
| AKT1 | Chr14: 104331625-104334625 | AKT1, Chr14: 104331625-104334625 |
| E2F1 | Chr20: 31736371-31739371 | E2F1, Chr20: 31736371-31739371 |
| DUSP15 | Chr20: 29920640-29923640 | DUSP15, Chr20: 29920640-29923640 |
| TRAF3IP1 | Chr2: 238892320-238895320 | TRAF3IP1, Chr2: 238892320-238895320 |
| FAM54B | Chr1: 26017483-26020483 | FAM54B, Chr1: 26017483-26020483 |
| CENPA | Chr2: 26860885-26863885 | CENPA, Chr2: 26860885-26863885 |
| CENPA | Chr2: 26860886-26863886 | CENPA, Chr2: 26860886-26863886 |
| ZNF251 | Chr8: 145950275-145953275 | ZNF251, Chr8: 145950275-145953275 |
| FAM129B | Chr9: 129369676-129372676 | FAM129B, Chr9: 129369676-129372676 |
| SYNGR2 | Chr17: 73674765-73677765 | SYNGR2, Chr17: 73674765-73677765 |
| TRIM41 | Chr5: 180581411-180584411 | TRIM41, Chr5: 180581411-180584411 |
| PPAN | Chr19: 10076464-10079464 | PPAN, Chr19: 10076464-10079464 |
| CRTC1 | Chr19: 18653924-18656924 | CRTC1, Chr19: 18653924-18656924 |
| SPEG | Chr2: 220006443-220009443 | SPEG, Chr2: 220006443-220009443 |
| ABT1 | Chr6: 26703658-26706658 | ABT1, Chr6: 26703658-26706658 |
| C10orf35 | Chr10: 71058508-71061508 | C10orf35, Chr10: 71058508-71061508 |
| CST6 | Chr11: 65534537-65537537 | CST6, Chr11: 65534537-65537537 |
| TMEM151A | Chr11: 65814416-65817416 | TMEM151A, Chr11: 65814416-65817416 |
| LOC402665 | Chr19: 56505413-56508413 | LOC402665, Chr19: 56505413-56508413 |
| LTBP4 | Chr19: 45797616-45800616 | LTBP4, Chr19: 45797616-45800616 |
| CANT1 | Chr17: 74515933-74518933 | CANT1, Chr17: 74515933-74518933 |
| PLCB3 | Chr11: 63774197-63777197 | PLCB3, Chr11: 63774197-63777197 |
| MAPK8IP1 | Chr11: 45862277-45865277 | MAPK8IP1, Chr11: 45862277-45865277 |
| BAX | Chr19: 54148428-54151428 | BAX, Chr19: 54148428-54151428 |
| KIAA1394 | Chr11: 66938224-66941224 | KIAA1394, Chr11: 66938224-66941224 |
| REEP2 | Chr5: 137801174-137804174 | REEP2, Chr5: 137801174-137804174 |
| LGR6 | Chr1: 200428240-200431240 | LGR6, Chr1: 200428240-200431240 |
| LHFPL5 | Chr6: 35879548-35882548 | LHFPL5, Chr6: 35879548-35882548 |
| PTPRE | Chr10: 129593814-129596814 | PTPRE, Chr10: 129593814-129596814 |
| CCND3 | Chr6: 42016030-42019030 | CCND3, Chr6: 42016030-42019030 |
| SEMA7A | Chr15: 72511829-72514829 | SEMA7A, Chr15: 72511829-72514829 |
| ID1 | Chr20: 29655252-29658252 | ID1, Chr20: 29655252-29658252 |
| ILVBL | Chr19: 15096077-15099077 | ILVBL, Chr19: 15096077-15099077 |
| FLJ10404 | Chr5: 176912644-176915644 | FLJ10404, Chr5: 176912644-176915644 |
| VAMP2 | Chr17: 8005518-8008518 | VAMP2, Chr17: 8005518-8008518 |
| SCRN2 | Chr17: 43272111-43275111 | SCRN2, Chr17: 43272111-43275111 |
| FAM29A | Chr9: 19091402-19094402 | FAM29A, Chr9: 19091402-19094402 |
| IFT74 | Chr9: 26935536-26938536 | IFT74, Chr9: 26935536-26938536 |
| BTBD8 | Chr1: 92316980-92319980 | BTBD8, Chr1: 92316980-92319980 |
| GSTO2 | Chr10: 106017120-106020120 | GSTO2, Chr10: 106017120-106020120 |
| ZNF593 | Chr1: 26367474-26370474 | ZNF593, Chr1: 26367474-26370474 |
| RABGGTA | Chr14: 23809143-23812143 | RABGGTA, Chr14: 23809143-23812143 |
| PADI2 | Chr1: 17317035-17320035 | PADI2, Chr1: 17317035-17320035 |
| DOK4 | Chr16: 56076386-56079386 | DOK4, Chr16: 56076386-56079386 |
| ELL3 | Chr15: 41855294-41858294 | ELL3, Chr15: 41855294-41858294 |
| CTSZ | Chr20: 57014180-57017180 | CTSZ, Chr20: 57014180-57017180 |
| C1orf67 | Chr1: 223182485-223185485 | C1orf67, Chr1: 223182485-223185485 |
| FAM109A | Chr12: 110289808-110292808 | FAM109A, Chr12: 110289808-110292808 |
| RABGGTA | Chr14: 23809083-23812083 | RABGGTA, Chr14: 23809083-23812083 |
| SNX19 | Chr11: 130290092-130293092 | SNX19, Chr11: 130290092-130293092 |
| RAB3B | Chr1: 52227436-52230436 | RAB3B, Chr1: 52227436-52230436 |
| GYPC | Chr2: 127128653-127131653 | GYPC, Chr2: 127128653-127131653 |
| ZFP36 | Chr19: 44587826-44590826 | ZFP36, Chr19: 44587826-44590826 |
| MYO18A | Chr17: 24530033-24533033 | MYO18A, Chr17: 24530033-24533033 |
| FBXO43 | Chr8: 101225752-101228752 | FBXO43, Chr8: 101225752-101228752 |
| GALK1 | Chr17: 71271375-71274375 | GALK1, Chr17: 71271375-71274375 |
| KIAA1257 | Chr3: 130194176-130197176 | KIAA1257, Chr3: 130194176-130197176 |
| 10-Mar | Chr17: 58237937-58240937 | 40612, Chr17: 58237937-58240937 |
| TXNRD2 | Chr22: 18307859-18310859 | TXNRD2, Chr22: 18307859-18310859 |
| UPP1 | Chr7: 48093770-48096770 | UPP1, Chr7: 48093770-48096770 |
| RBM3 | Chr23: 48316279-48319279 | RBM3, Chr23: 48316279-48319279 |
| PRODH | Chr22: 17302314-17305314 | PRODH, Chr22: 17302314-17305314 |
| CORO1B | Chr11: 66966339-66969339 | CORO1B, Chr11: 66966339-66969339 |
| CARD11 | Chr7: 3048605-3051605 | CARD11, Chr7: 3048605-3051605 |
| LSR | Chr19: 40429898-40432898 | LSR, Chr19: 40429898-40432898 |
| TNFRSF13C | Chr22: 40651267-40654267 | TNFRSF13C, Chr22: 40651267-40654267 |
| IQCF2 | Chr3: 51869184-51872184 | IQCF2, Chr3: 51869184-51872184 |
| SPON1 | Chr11: 13938989-13941989 | SPON1, Chr11: 13938989-13941989 |
| CMTM5 | Chr14: 22914356-22917356 | CMTM5, Chr14: 22914356-22917356 |
| SLC41A3 | Chr3: 127256808-127259808 | SLC41A3, Chr3: 127256808-127259808 |
| C1QA | Chr1: 22834204-22837204 | C1QA, Chr1: 22834204-22837204 |
| DHX58 | Chr17: 37516777-37519777 | DHX58, Chr17: 37516777-37519777 |
| ZNF185 | Chr23: 151832152-151835152 | ZNF185, Chr23: 151832152-151835152 |
| RHO | Chr3: 130728671-130731671 | RHO, Chr3: 130728671-130731671 |
| TCF7 | Chr5: 133476800-133479800 | TCF7, Chr5: 133476800-133479800 |
| FREQ | Chr9: 132001192-132004192 | FREQ, Chr9: 132001192-132004192 |
| ABCG1 | Chr21: 42507755-42510755 | ABCG1, Chr21: 42507755-42510755 |
| MMRN2 | Chr10: 88705852-88708852 | MMRN2, Chr10: 88705852-88708852 |
| CUEDC1 | Chr17: 53334249-53337249 | CUEDC1, Chr17: 53334249-53337249 |
| MT1G | Chr16: 55257978-55260978 | MT1G, Chr16: 55257978-55260978 |
| CALML4 | Chr15: 66284002-66287002 | CALML4, Chr15: 66284002-66287002 |
| CENTD2 | Chr11: 72109528-72112528 | CENTD2, Chr11: 72109528-72112528 |
| C2orf50 | Chr2: 11189129-11192129 | C2orf50, Chr2: 11189129-11192129 |
| USP19 | Chr3: 49131717-49134717 | USP19, Chr3: 49131717-49134717 |
| SPTB | Chr14: 64358119-64361119 | SPTB, Chr14: 64358119-64361119 |
| ALLC | Chr2: 3682160-3685160 | ALLC, Chr2: 3682160-3685160 |
| ITIH3 | Chr3: 52802323-52805323 | ITIH3, Chr3: 52802323-52805323 |
| NDUFS2 | Chr1: 159434228-159437228 | NDUFS2, Chr1: 159434228-159437228 |
| OSTalpha | Chr3: 197426279-197429279 | OSTalpha, Chr3: 197426279-197429279 |
| C10orf33 | Chr10: 100163431-100166431 | C10orf33, Chr10: 100163431-100166431 |
| OSGIN1 | Chr16: 82538672-82541672 | OSGIN1, Chr16: 82538672-82541672 |
| AXL | Chr19: 46415447-46418447 | AXL, Chr19: 46415447-46418447 |
| CHRNA2 | Chr8: 27391230-27394230 | CHRNA2, Chr8: 27391230-27394230 |
| TCP10L | Chr21: 32878214-32881214 | TCP10L, Chr21: 32878214-32881214 |
| PLEKHA6 | Chr1: 202594167-202597167 | PLEKHA6, Chr1: 202594167-202597167 |
| P2RY6 | Chr11: 72657086-72660086 | P2RY6, Chr11: 72657086-72660086 |
| IL11RA | Chr9: 34642431-34645431 | IL11RA, Chr9: 34642431-34645431 |
| ORAI3 | Chr16: 30866415-30869415 | ORAI3, Chr16: 30866415-30869415 |
| TCF7 | Chr5: 133477714-133480714 | TCF7, Chr5: 133477714-133480714 |
| P2RY6 | Chr11: 72659394-72662394 | P2RY6, Chr11: 72659394-72662394 |
| DNAJB8 | Chr3: 129667281-129670281 | DNAJB8, Chr3: 129667281-129670281 |
| USP6 | Chr17: 4970910-4973910 | USP6, Chr17: 4970910-4973910 |
| ATAD4 | Chr17: 43388609-43391609 | ATAD4, Chr17: 43388609-43391609 |
| CCDC33 | Chr15: 72314219-72317219 | CCDC33, Chr15: 72314219-72317219 |
| CARD14 | Chr17: 75774693-75777693 | CARD14, Chr17: 75774693-75777693 |
| MATK | Chr19: 3751310-3754310 | MATK, Chr19: 3751310-3754310 |
| RPH3AL | Chr17: 201076-204076 | RPH3AL, Chr17: 201076-204076 |
| OPN4 | Chr10: 88402793-88405793 | OPN4, Chr10: 88402793-88405793 |
| COX19 | Chr7: 980261-983261 | COX19, Chr7: 980261-983261 |
| ARTN | Chr1: 44170078-44173078 | ARTN, Chr1: 44170078-44173078 |
| STH | Chr17: 41430952-41433952 | STH, Chr17: 41430952-41433952 |
| CEACAM8 | Chr19: 47789390-47792390 | CEACAM8, Chr19: 47789390-47792390 |
| ODF3L1 | Chr15: 73801873-73804873 | ODF3L1, Chr15: 73801873-73804873 |
| C21orf29 | Chr21: 44954423-44957423 | C21orf29, Chr21: 44954423-44957423 |
| SMYD3 | Chr1: 244645834-244648834 | SMYD3, Chr1: 244645834-244648834 |
| NUBPL | Chr14: 31098841-31101841 | NUBPL, Chr14: 31098841-31101841 |
| PLEKHG5 | Chr1: 6471727-6474727 | PLEKHG5, Chr1: 6471727-6474727 |
| PSG8 | Chr19: 47960073-47963073 | PSG8, Chr19: 47960073-47963073 |
| COL16A1 | Chr1: 31940855-31943855 | COL16A1, Chr1: 31940855-31943855 |
| TBC1D28 | Chr17: 18486965-18489965 | TBC1D28, Chr17: 18486965-18489965 |
| GNGT2 | Chr17: 44640242-44643242 | GNGT2, Chr17: 44640242-44643242 |
| RNF167 | Chr17: 4782874-4785874 | RNF167, Chr17: 4782874-4785874 |
| ZNHIT1 | Chr7: 100646204-100649204 | ZNHIT1, Chr7: 100646204-100649204 |
| LRRC29 | Chr16: 65816902-65819902 | LRRC29, Chr16: 65816902-65819902 |
| PSG7 | Chr19: 48131670-48134670 | PSG7, Chr19: 48131670-48134670 |
| VDAC1 | Chr5: 133366832-133369832 | VDAC1, Chr5: 133366832-133369832 |
| RAB17 | Chr2: 238162975-238165975 | RAB17, Chr2: 238162975-238165975 |
| ATP6V0D1 | Chr16: 66071090-66074090 | ATP6V0D1, Chr16: 66071090-66074090 |
| IL16 | Chr15: 79274773-79277773 | IL16, Chr15: 79274773-79277773 |
| HTR1F | Chr3: 88112915-88115915 | HTR1F, Chr3: 88112915-88115915 |
| SYNE2 | Chr14: 63750924-63753924 | SYNE2, Chr14: 63750924-63753924 |
| NLRP12 | Chr19: 59002271-59005271 | NLRP12, Chr19: 59002271-59005271 |
| APOE | Chr19: 50099378-50102378 | APOE, Chr19: 50099378-50102378 |
| COL3A1 | Chr2: 189545843-189548843 | COL3A1, Chr2: 189545843-189548843 |
| TXNDC17 | Chr17: 6483445-6486445 | TXNDC17, Chr17: 6483445-6486445 |
| KCNJ4 | Chr22: 37168479-37171479 | KCNJ4, Chr22: 37168479-37171479 |
| TYROBP | Chr19: 41089526-41092526 | TYROBP, Chr19: 41089526-41092526 |
| GPR173 | Chr23: 53093730-53096730 | GPR173, Chr23: 53093730-53096730 |
| SULT1A1 | Chr16: 28526650-28529650 | SULT1A1, Chr16: 28526650-28529650 |
| TAGLN | Chr11: 116573749-116576749 | TAGLN, Chr11: 116573749-116576749 |
| NR1H3 | Chr11: 47235808-47238808 | NR1H3, Chr11: 47235808-47238808 |
| CACNG7 | Chr19: 59106382-59109382 | CACNG7, Chr19: 59106382-59109382 |
| RAC2 | Chr22: 35968751-35971751 | RAC2, Chr22: 35968751-35971751 |
| KCNMB1 | Chr5: 169747716-169750716 | KCNMB1, Chr5: 169747716-169750716 |
| TRPV1 | Chr17: 3457954-3460954 | TRPV1, Chr17: 3457954-3460954 |
| SGSH | Chr17: 75807294-75810294 | SGSH, Chr17: 75807294-75810294 |
| MYH6 | Chr14: 22945822-22948822 | MYH6, Chr14: 22945822-22948822 |
| FLJ23834 | Chr7: 105389392-105392392 | FLJ23834, Chr7: 105389392-105392392 |
| LOC728137 | Chr24: 9294548-9297548 | LOC728137, Chr24: 9294548-9297548 |
| OR6N2 | Chr1: 157012549-157015549 | OR6N2, Chr1: 157012549-157015549 |
| ADAMTS5 | Chr21: 27259203-27262203 | ADAMTS5, Chr21: 27259203-27262203 |
| LCAP | Chr23: 152797820-152800820 | LCAP, Chr23: 152797820-152800820 |
| EPS8L3 | Chr1: 110106587-110109587 | EPS8L3, Chr1: 110106587-110109587 |
| CYB561 | Chr17: 58870439-58873439 | CYB561, Chr17: 58870439-58873439 |
| SEBOX | Chr17: 23717614-23720614 | SEBOX, Chr17: 23717614-23720614 |
| SULT1A2 | Chr16: 28513802-28516802 | SULT1A2, Chr16: 28513802-28516802 |
| CCDC28B | Chr1: 32437288-32440288 | CCDC28B, Chr1: 32437288-32440288 |
| INMT | Chr7: 30756775-30759775 | INMT, Chr7: 30756775-30759775 |
| SPACA3 | Chr17: 28341494-28344494 | SPACA3, Chr17: 28341494-28344494 |
| PPP2R2D | Chr10: 133596449-133599449 | PPP2R2D, Chr10: 133596449-133599449 |
| LRRC8E | Chr19: 7857889-7860889 | LRRC8E, Chr19: 7857889-7860889 |
| ALS2CL | Chr3: 46692403-46695403 | ALS2CL, Chr3: 46692403-46695403 |
| DMKN | Chr19: 40694900-40697900 | DMKN, Chr19: 40694900-40697900 |
| C22orf9 | Chr22: 43985511-43988511 | C22orf9, Chr22: 43985511-43988511 |
| IL21R | Chr16: 27320423-27323423 | IL21R, Chr16: 27320423-27323423 |
| C10orf71 | Chr10: 50175692-50178692 | C10orf71, Chr10: 50175692-50178692 |
| SFXN3 | Chr10: 102779485-102782485 | SFXN3, Chr10: 102779485-102782485 |
| ZNF385A | Chr12: 53063248-53066248 | ZNF385A, Chr12: 53063248-53066248 |
| TSPAN10 | Chr17: 77218253-77221253 | TSPAN10, Chr17: 77218253-77221253 |
| SERPINF2 | Chr17: 1591379-1594379 | SERPINF2, Chr17: 1591379-1594379 |
| P4HA2 | Chr5: 131589955-131592955 | P4HA2, Chr5: 131589955-131592955 |
| TACR2 | Chr10: 70845180-70848180 | TACR2, Chr10: 70845180-70848180 |
| C10orf65 | Chr10: 99332610-99335610 | C10orf65, Chr10: 99332610-99335610 |
| TFF2 | Chr21: 42642676-42645676 | TFF2, Chr21: 42642676-42645676 |
| PDZD7 | Chr10: 102779369-102782369 | PDZD7, Chr10: 102779369-102782369 |
| DCST1 | Chr1: 153271423-153274423 | DCST1, Chr1: 153271423-153274423 |
| ARHGEF7 | Chr13: 110635673-110638673 | ARHGEF7, Chr13: 110635673-110638673 |
| LOC645296 | Chr15: 69419640-69422640 | LOC645296, Chr15: 69419640-69422640 |
| PTPN6 | Chr12: 6924500-6927500 | PTPN6, Chr12: 6924500-6927500 |
| GRB7 | Chr17: 35146615-35149615 | GRB7, Chr17: 35146615-35149615 |
| FCN2 | Chr9: 136910978-136913978 | FCN2, Chr9: 136910978-136913978 |
| RFPL3 | Chr22: 31082353-31085353 | RFPL3, Chr22: 31082353-31085353 |
| LOC51252 | Chr2: 96926058-96929058 | LOC51252, Chr2: 96926058-96929058 |
| SLC6A12 | Chr12: 191253-194253 | SLC6A12, Chr12: 191253-194253 |
| RAB11FIP4 | Chr17: 26741267-26744267 | RAB11FIP4, Chr17: 26741267-26744267 |
| INHBE | Chr12: 56133862-56136862 | INHBE, Chr12: 56133862-56136862 |
| ASB2 | Chr14: 93492020-93495020 | ASB2, Chr14: 93492020-93495020 |
| C20orf197 | Chr20: 58062874-58065874 | C20orf197, Chr20: 58062874-58065874 |
| IL3 | Chr5: 131422745-131425745 | IL3, Chr5: 131422745-131425745 |
| CAV3 | Chr3: 8748995-8751995 | CAV3, Chr3: 8748995-8751995 |
| DHRS2 | Chr14: 23173912-23176912 | DHRS2, Chr14: 23173912-23176912 |
| CALN1 | Chr7: 71513796-71516796 | CALN1, Chr7: 71513796-71516796 |
| C16orf70 | Chr16: 65699915-65702915 | C16orf70, Chr16: 65699915-65702915 |
| ZNF626 | Chr19: 20634742-20637742 | ZNF626, Chr19: 20634742-20637742 |
| TBC1D29 | Chr17: 25909209-25912209 | TBC1D29, Chr17: 25909209-25912209 |
| PAQR6 | Chr1: 154482967-154485967 | PAQR6, Chr1: 154482967-154485967 |
| FRZB | Chr2: 183438243-183441243 | FRZB, Chr2: 183438243-183441243 |
| HVCN1 | Chr12: 109609829-109612829 | HVCN1, Chr12: 109609829-109612829 |
| PAQR6 | Chr1: 154482916-154485916 | PAQR6, Chr1: 154482916-154485916 |
| ZC3H12D | Chr6: 149846223-149849223 | ZC3H12D, Chr6: 149846223-149849223 |
| WDR5 | Chr9: 135993234-135996234 | WDR5, Chr9: 135993234-135996234 |
| RASSF2 | Chr20: 4742269-4745269 | RASSF2, Chr20: 4742269-4745269 |
| WSCD1 | Chr17: 5913157-5916157 | WSCD1, Chr17: 5913157-5916157 |
| ANKK1 | Chr11: 112762222-112765222 | ANKK1, Chr11: 112762222-112765222 |
| SLC38A8 | Chr16: 82631763-82634763 | SLC38A8, Chr16: 82631763-82634763 |
| KIAA1609 | Chr16: 83094294-83097294 | KIAA1609, Chr16: 83094294-83097294 |
| PACSIN2 | Chr22: 41671491-41674491 | PACSIN2, Chr22: 41671491-41674491 |
| MMP26 | Chr11: 4964499-4967499 | MMP26, Chr11: 4964499-4967499 |
| GPR56 | Chr16: 56218522-56221522 | GPR56, Chr16: 56218522-56221522 |
| GPR56 | Chr16: 56218541-56221541 | GPR56, Chr16: 56218541-56221541 |
| LOC391343 | Chr2: 894511-897511 | LOC391343, Chr2: 894511-897511 |
| ANXA9 | Chr1: 149219622-149222622 | ANXA9, Chr1: 149219622-149222622 |
| PLA2G2E | Chr1: 20121197-20124197 | PLA2G2E, Chr1: 20121197-20124197 |
| EPHX1 | Chr1: 224078098-224081098 | EPHX1, Chr1: 224078098-224081098 |
| SIGLEC9 | Chr19: 56318476-56321476 | SIGLEC9, Chr19: 56318476-56321476 |
| DHCR7 | Chr11: 70835625-70838625 | DHCR7, Chr11: 70835625-70838625 |
| GAGE12F | Chr23: 49103214-49106214 | GAGE12F, Chr23: 49103214-49106214 |
| TBC1D26 | Chr17: 15574815-15577815 | TBC1D26, Chr17: 15574815-15577815 |
| SGK2 | Chr20: 41626650-41629650 | SGK2, Chr20: 41626650-41629650 |
| TNFRSF8 | Chr1: 12107044-12110044 | TNFRSF8, Chr1: 12107044-12110044 |
| 9-Sep | Chr17: 72981419-72984419 | 40795, Chr17: 72981419-72984419 |
| SLC6A12 | Chr12: 191595-194595 | SLC6A12, Chr12: 191595-194595 |
| SULT1A1 | Chr16: 28527358-28530358 | SULT1A1, Chr16: 28527358-28530358 |
| SHISA4 | Chr1: 200123154-200126154 | SHISA4, Chr1: 200123154-200126154 |
| SULT1A2 | Chr16: 28514392-28517392 | SULT1A2, Chr16: 28514392-28517392 |
| PRKACA | Chr19: 14084492-14087492 | PRKACA, Chr19: 14084492-14087492 |
| SLC5A11 | Chr16: 24763552-24766552 | SLC5A11, Chr16: 24763552-24766552 |
| S100A14 | Chr1: 151853914-151856914 | S100A14, Chr1: 151853914-151856914 |
| KLK14 | Chr19: 56277814-56280814 | KLK14, Chr19: 56277814-56280814 |
| GRB7 | Chr17: 35146212-35149212 | GRB7, Chr17: 35146212-35149212 |
| PEBP4 | Chr8: 22839866-22842866 | PEBP4, Chr8: 22839866-22842866 |
| CLPS | Chr6: 35871580-35874580 | CLPS, Chr6: 35871580-35874580 |
| HSPB2 | Chr11: 111287208-111290208 | HSPB2, Chr11: 111287208-111290208 |
| LCN1 | Chr9: 137551606-137554606 | LCN1, Chr9: 137551606-137554606 |
| OBP2B | Chr9: 135072949-135075949 | OBP2B, Chr9: 135072949-135075949 |
| C8orf22 | Chr8: 50145955-50148955 | C8orf22, Chr8: 50145955-50148955 |
| PTGES | Chr9: 131553665-131556665 | PTGES, Chr9: 131553665-131556665 |
| CRYBB2 | Chr22: 23944111-23947111 | CRYBB2, Chr22: 23944111-23947111 |
| LOC339047 | Chr16: 18376697-18379697 | LOC339047, Chr16: 18376697-18379697 |
| CRYGN | Chr7: 150766532-150769532 | CRYGN, Chr7: 150766532-150769532 |
| C10orf72 | Chr10: 49992065-49995065 | C10orf72, Chr10: 49992065-49995065 |
| CLUAP1 | Chr16: 3498394-3501394 | CLUAP1, Chr16: 3498394-3501394 |
| LRTM2 | Chr12: 1798455-1801455 | LRTM2, Chr12: 1798455-1801455 |
| RNF112 | Chr17: 19253615-19256615 | RNF112, Chr17: 19253615-19256615 |
| KRT79 | Chr12: 51512844-51515844 | KRT79, Chr12: 51512844-51515844 |
| BHMT | Chr5: 78441859-78444859 | BHMT, Chr5: 78441859-78444859 |
| IL21R | Chr16: 27319723-27322723 | IL21R, Chr16: 27319723-27322723 |
| CYP4F12 | Chr19: 15643385-15646385 | CYP4F12, Chr19: 15643385-15646385 |
| ART3 | Chr4: 77149860-77152860 | ART3, Chr4: 77149860-77152860 |
| VPREB3 | Chr22: 22425092-22428092 | VPREB3, Chr22: 22425092-22428092 |
| GREB1 | Chr2: 11596030-11599030 | GREB1, Chr2: 11596030-11599030 |
| TRIOBP | Chr22: 36422073-36425073 | TRIOBP, Chr22: 36422073-36425073 |
| SUMO4 | Chr6: 149761687-149764687 | SUMO4, Chr6: 149761687-149764687 |
| GCK | Chr7: 44194047-44197047 | GCK, Chr7: 44194047-44197047 |
| MT4 | Chr16: 55154961-55157961 | MT4, Chr16: 55154961-55157961 |
| PRF1 | Chr10: 72031037-72034037 | PRF1, Chr10: 72031037-72034037 |
| TPD52L3 | Chr9: 6316874-6319874 | TPD52L3, Chr9: 6316874-6319874 |
| LOC441294 | Chr7: 142977511-142980511 | LOC441294, Chr7: 142977511-142980511 |
| TEX101 | Chr19: 48609374-48612374 | TEX101, Chr19: 48609374-48612374 |
| C10orf27 | Chr10: 72213663-72216663 | C10orf27, Chr10: 72213663-72216663 |
| CSAG1 | Chr23: 151652383-151655383 | CSAG1, Chr23: 151652383-151655383 |
| FAM101B | Chr17: 294459-297459 | FAM101B, Chr17: 294459-297459 |
| DNASE1L1 | Chr23: 153289269-153292269 | DNASE1L1, Chr23: 153289269-153292269 |
| MYH7 | Chr14: 22973210-22976210 | MYH7, Chr14: 22973210-22976210 |
| ZNF558 | Chr19: 8793065-8796065 | ZNF558, Chr19: 8793065-8796065 |
| KLHL28 | Chr14: 44499429-44502429 | KLHL28, Chr14: 44499429-44502429 |
| GGT1 | Chr22: 23332154-23335154 | GGT1, Chr22: 23332154-23335154 |
| PEMT | Chr17: 17420004-17423004 | PEMT, Chr17: 17420004-17423004 |
| FAM166B | Chr9: 35552396-35555396 | FAM166B, Chr9: 35552396-35555396 |
| LCTL | Chr15: 64643389-64646389 | LCTL, Chr15: 64643389-64646389 |
| GPR114 | Chr16: 56132601-56135601 | GPR114, Chr16: 56132601-56135601 |
| PLA2G2F | Chr1: 20336909-20339909 | PLA2G2F, Chr1: 20336909-20339909 |
| ACCN1 | Chr17: 29506438-29509438 | ACCN1, Chr17: 29506438-29509438 |
| POU2AF1 | Chr11: 110753867-110756867 | POU2AF1, Chr11: 110753867-110756867 |
| HNF4A | Chr20: 42416354-42419354 | HNF4A, Chr20: 42416354-42419354 |
| COLEC11 | Chr2: 3619011-3622011 | COLEC11, Chr2: 3619011-3622011 |
| TREM2 | Chr6: 41237400-41240400 | TREM2, Chr6: 41237400-41240400 |
| ZACN | Chr17: 71585403-71588403 | ZACN, Chr17: 71585403-71588403 |
| NCR2 | Chr6: 41410005-41413005 | NCR2, Chr6: 41410005-41413005 |
| GSDM1 | Chr17: 35371251-35374251 | GSDM1, Chr17: 35371251-35374251 |
| ASGR2 | Chr17: 6956853-6959853 | ASGR2, Chr17: 6956853-6959853 |
| CACNG5 | Chr17: 62302412-62305412 | CACNG5, Chr17: 62302412-62305412 |
| PTPRH | Chr19: 60411154-60414154 | PTPRH, Chr19: 60411154-60414154 |
| DMBX1 | Chr1: 46743754-46746754 | DMBX1, Chr1: 46743754-46746754 |
| KSP37 | Chr10: 44725269-44728269 | KSP37, Chr10: 44725269-44728269 |
| ADIG | Chr20: 36641751-36644751 | ADIG, Chr20: 36641751-36644751 |
| SRL | Chr16: 4230582-4233582 | SRL, Chr16: 4230582-4233582 |
| ABCG5 | Chr2: 43917962-43920962 | ABCG5, Chr2: 43917962-43920962 |
| ZDHHC4 | Chr7: 6583166-6586166 | ZDHHC4, Chr7: 6583166-6586166 |
| CNDP1 | Chr18: 70351171-70354171 | CNDP1, Chr18: 70351171-70354171 |
| HVCN1 | Chr12: 109610500-109613500 | HVCN1, Chr12: 109610500-109613500 |
| LRP5L | Chr22: 24087024-24090024 | LRP5L, Chr22: 24087024-24090024 |
| 12-Sep | Chr16: 4776848-4779848 | 40798, Chr16: 4776848-4779848 |
| ANGPTL2 | Chr9: 128923365-128926365 | ANGPTL2, Chr9: 128923365-128926365 |
| CSF2 | Chr5: 131435883-131438883 | CSF2, Chr5: 131435883-131438883 |
| KRT72 | Chr12: 51280059-51283059 | KRT72, Chr12: 51280059-51283059 |
| FLJ43752 | Chr6: 33667593-33670593 | FLJ43752, Chr6: 33667593-33670593 |
| CRYBB3 | Chr22: 23924324-23927324 | CRYBB3, Chr22: 23924324-23927324 |
| ABCB9 | Chr12: 122015509-122018509 | ABCB9, Chr12: 122015509-122018509 |
| HIST1H1A | Chr6: 26124519-26127519 | HIST1H1A, Chr6: 26124519-26127519 |
| PADI3 | Chr1: 17446679-17449679 | PADI3, Chr1: 17446679-17449679 |
| H1FOO | Chr3: 130743246-130746246 | H1FOO, Chr3: 130743246-130746246 |
| WISP1 | Chr8: 134270993-134273993 | WISP1, Chr8: 134270993-134273993 |
| LMAN1L | Chr15: 72890746-72893746 | LMAN1L, Chr15: 72890746-72893746 |
| HMSD | Chr18: 59766067-59769067 | HMSD, Chr18: 59766067-59769067 |
| IHPK3 | Chr6: 33821160-33824160 | IHPK3, Chr6: 33821160-33824160 |
| COX4I2 | Chr20: 29687851-29690851 | COX4I2, Chr20: 29687851-29690851 |
| THRSP | Chr11: 77451054-77454054 | THRSP, Chr11: 77451054-77454054 |
| VPREB1 | Chr22: 20927699-20930699 | VPREB1, Chr22: 20927699-20930699 |
| PADI6 | Chr1: 17569827-17572827 | PADI6, Chr1: 17569827-17572827 |
| ATP6V1B1 | Chr2: 71015005-71018005 | ATP6V1B1, Chr2: 71015005-71018005 |
| ZPBP | Chr7: 50101872-50104872 | ZPBP, Chr7: 50101872-50104872 |
| SLC7A7 | Chr14: 22357354-22360354 | SLC7A7, Chr14: 22357354-22360354 |
| GHRHR | Chr7: 30968660-30971660 | GHRHR, Chr7: 30968660-30971660 |
| UGT1A3 | Chr2: 234301011-234304011 | UGT1A3, Chr2: 234301011-234304011 |
| C1QB | Chr1: 22850768-22853768 | C1QB, Chr1: 22850768-22853768 |
| CTAGE1 | Chr18: 18250376-18253376 | CTAGE1, Chr18: 18250376-18253376 |
| ATOH1 | Chr4: 94967600-94970600 | ATOH1, Chr4: 94967600-94970600 |
| MS4A13 | Chr11: 60037961-60040961 | MS4A13, Chr11: 60037961-60040961 |
| SHBG | Chr17: 7472715-7475715 | SHBG, Chr17: 7472715-7475715 |
| S100B | Chr21: 46847963-46850963 | S100B, Chr21: 46847963-46850963 |
| GJB1 | Chr23: 70350286-70353286 | GJB1, Chr23: 70350286-70353286 |
| ABCG8 | Chr2: 43918106-43921106 | ABCG8, Chr2: 43918106-43921106 |
| TNS4 | Chr17: 35909880-35912880 | TNS4, Chr17: 35909880-35912880 |
| FBXL22 | Chr15: 61675148-61678148 | FBXL22, Chr15: 61675148-61678148 |
| MBP | Chr18: 72856543-72859543 | MBP, Chr18: 72856543-72859543 |
| MAGEA2 | Chr23: 151632274-151635274 | MAGEA2, Chr23: 151632274-151635274 |
| SLC38A5 | Chr23: 48212088-48215088 | SLC38A5, Chr23: 48212088-48215088 |
| TMEM125 | Chr1: 43507493-43510493 | TMEM125, Chr1: 43507493-43510493 |
| ITGA4 | Chr2: 182028363-182031363 | ITGA4, Chr2: 182028363-182031363 |
| CRIP3 | Chr6: 43383008-43386008 | CRIP3, Chr6: 43383008-43386008 |
| LGI4 | Chr19: 40316518-40319518 | LGI4, Chr19: 40316518-40319518 |
| C11orf66 | Chr11: 61003667-61006667 | C11orf66, Chr11: 61003667-61006667 |
| CEL | Chr9: 134925685-134928685 | CEL, Chr9: 134925685-134928685 |
| OR5C1 | Chr9: 124589532-124592532 | OR5C1, Chr9: 124589532-124592532 |
| TCTEX1D1 | Chr1: 66989229-66992229 | TCTEX1D1, Chr1: 66989229-66992229 |
| TMEM162 | Chr19: 40409968-40412968 | TMEM162, Chr19: 40409968-40412968 |
| ZDHHC19 | Chr3: 197421197-197424197 | ZDHHC19, Chr3: 197421197-197424197 |
| GIMAP8 | Chr7: 149777394-149780394 | GIMAP8, Chr7: 149777394-149780394 |
| IL2RB | Chr22: 35874408-35877408 | IL2RB, Chr22: 35874408-35877408 |
| ATXN7L3 | Chr17: 39629555-39632555 | ATXN7L3, Chr17: 39629555-39632555 |
| BSND | Chr1: 55235704-55238704 | BSND, Chr1: 55235704-55238704 |
| PRM1 | Chr16: 11281193-11284193 | PRM1, Chr16: 11281193-11284193 |
| PON1 | Chr7: 94790280-94793280 | PON1, Chr7: 94790280-94793280 |
| LOC388381 | Chr17: 34249668-34252668 | LOC388381, Chr17: 34249668-34252668 |
| KLHDC8A | Chr1: 203591162-203594162 | KLHDC8A, Chr1: 203591162-203594162 |
| NUP210L | Chr1: 152392716-152395716 | NUP210L, Chr1: 152392716-152395716 |
| SPRR2G | Chr1: 151387731-151390731 | SPRR2G, Chr1: 151387731-151390731 |
| CLDN18 | Chr3: 139198847-139201847 | CLDN18, Chr3: 139198847-139201847 |
| TNS1 | Chr2: 218515541-218518541 | TNS1, Chr2: 218515541-218518541 |
| KIAA1267 | Chr17: 41603871-41606871 | KIAA1267, Chr17: 41603871-41606871 |
| TMPRSS4 | Chr11: 117451436-117454436 | TMPRSS4, Chr11: 117451436-117454436 |
| HIST1H4I | Chr6: 27213566-27216566 | HIST1H4I, Chr6: 27213566-27216566 |
| NPSR1 | Chr7: 34662921-34665921 | NPSR1, Chr7: 34662921-34665921 |
| VIL1 | Chr2: 218995223-218998223 | VIL1, Chr2: 218995223-218998223 |
| CARD14 | Chr17: 75765375-75768375 | CARD14, Chr17: 75765375-75768375 |
| LILRA4 | Chr19: 59540733-59543733 | LILRA4, Chr19: 59540733-59543733 |
| MUC17 | Chr7: 100448583-100451583 | MUC17, Chr7: 100448583-100451583 |
| TULP1 | Chr6: 35587125-35590125 | TULP1, Chr6: 35587125-35590125 |
| SAGE1 | Chr23: 134801950-134804950 | SAGE1, Chr23: 134801950-134804950 |
| LOC646227 | Chr5: 180340323-180343323 | LOC646227, Chr5: 180340323-180343323 |
| STC1 | Chr8: 23766765-23769765 | STC1, Chr8: 23766765-23769765 |
| TM4SF19 | Chr3: 197548155-197551155 | TM4SF19, Chr3: 197548155-197551155 |
| DAPL1 | Chr2: 159358586-159361586 | DAPL1, Chr2: 159358586-159361586 |
| PIB5PA | Chr22: 29847460-29850460 | PIB5PA, Chr22: 29847460-29850460 |
| SLC6A12 | Chr12: 192132-195132 | SLC6A12, Chr12: 192132-195132 |
| PRR5 | Chr22: 43449851-43452851 | PRR5, Chr22: 43449851-43452851 |
| CROCC | Chr1: 17119531-17122531 | CROCC, Chr1: 17119531-17122531 |
| IKBKE | Chr1: 204708918-204711918 | IKBKE, Chr1: 204708918-204711918 |
| BCL6B | Chr17: 6865592-6868592 | BCL6B, Chr17: 6865592-6868592 |
| 9-Sep | Chr17: 72825691-72828691 | 40795, Chr17: 72825691-72828691 |
| TSGA10IP | Chr11: 65468190-65471190 | TSGA10IP, Chr11: 65468190-65471190 |
| KCNIP2 | Chr10: 103592167-103595167 | KCNIP2, Chr10: 103592167-103595167 |
| NOTCH4 | Chr6: 32298322-32301322 | NOTCH4, Chr6: 32298322-32301322 |
| TIMP1 | Chr23: 47325133-47328133 | TIMP1, Chr23: 47325133-47328133 |
| CCDC27 | Chr1: 3657321-3660321 | CCDC27, Chr1: 3657321-3660321 |
| PAGE1 | Chr23: 49345807-49348807 | PAGE1, Chr23: 49345807-49348807 |
| FFAR2 | Chr19: 40630956-40633956 | FFAR2, Chr19: 40630956-40633956 |
| HSPA12B | Chr20: 3659855-3662855 | HSPA12B, Chr20: 3659855-3662855 |
| PF4 | Chr4: 75065079-75068079 | PF4, Chr4: 75065079-75068079 |
| SDSL | Chr12: 112343098-112346098 | SDSL, Chr12: 112343098-112346098 |
| TCF7 | Chr5: 133477825-133480825 | TCF7, Chr5: 133477825-133480825 |
| ENTHD1 | Chr22: 38618240-38621240 | ENTHD1, Chr22: 38618240-38621240 |
| TNFSF15 | Chr9: 116606729-116609729 | TNFSF15, Chr9: 116606729-116609729 |
| RUNX1T1 | Chr8: 93142867-93145867 | RUNX1T1, Chr8: 93142867-93145867 |
| GFRA4 | Chr20: 3590546-3593546 | GFRA4, Chr20: 3590546-3593546 |
| PDZK1IP1 | Chr1: 47426858-47429858 | PDZK1IP1, Chr1: 47426858-47429858 |
| TRIM10 | Chr6: 30235190-30238190 | TRIM10, Chr6: 30235190-30238190 |
| GALM | Chr2: 38745242-38748242 | GALM, Chr2: 38745242-38748242 |
| SELPLG | Chr12: 107550299-107553299 | SELPLG, Chr12: 107550299-107553299 |
| PAGE5 | Chr23: 55262015-55265015 | PAGE5, Chr23: 55262015-55265015 |
| PAGE5 | Chr23: 55262016-55265016 | PAGE5, Chr23: 55262016-55265016 |
| KRTAP3-3 | Chr17: 36402411-36405411 | KRTAP3-3, Chr17: 36402411-36405411 |
| IL12RB1 | Chr19: 18057197-18060197 | IL12RB1, Chr19: 18057197-18060197 |
| ITIH1 | Chr3: 52785147-52788147 | ITIH1, Chr3: 52785147-52788147 |
| ADAMTS13 | Chr9: 135275440-135278440 | ADAMTS13, Chr9: 135275440-135278440 |
| BMF | Chr15: 38184079-38187079 | BMF, Chr15: 38184079-38187079 |
| CXorf48 | Chr23: 134131326-134134326 | CXorf48, Chr23: 134131326-134134326 |
| CST2 | Chr20: 23753812-23756812 | CST2, Chr20: 23753812-23756812 |
| PNLIPRP2 | Chr10: 118368954-118371954 | PNLIPRP2, Chr10: 118368954-118371954 |
| LYPLAL1 | Chr1: 217412314-217415314 | LYPLAL1, Chr1: 217412314-217415314 |
| KIAA0423 | Chr14: 44499665-44502665 | KIAA0423, Chr14: 44499665-44502665 |
| HTR3A | Chr11: 113349619-113352619 | HTR3A, Chr11: 113349619-113352619 |
| KLK3 | Chr19: 56048482-56051482 | KLK3, Chr19: 56048482-56051482 |
| SLC5A10 | Chr17: 18794702-18797702 | SLC5A10, Chr17: 18794702-18797702 |
| PPY | Chr17: 39373859-39376859 | PPY, Chr17: 39373859-39376859 |
| FAM167B | Chr1: 32483904-32486904 | FAM167B, Chr1: 32483904-32486904 |
| ZFP57 | Chr6: 29755366-29758366 | ZFP57, Chr6: 29755366-29758366 |
| KRT2 | Chr12: 51330726-51333726 | KRT2, Chr12: 51330726-51333726 |
| OR1K1 | Chr9: 124600722-124603722 | OR1K1, Chr9: 124600722-124603722 |
| GOLT1A | Chr1: 202448343-202451343 | GOLT1A, Chr1: 202448343-202451343 |
| GPR81 | Chr12: 121779582-121782582 | GPR81, Chr12: 121779582-121782582 |
| LSDP5 | Chr19: 4484708-4487708 | LSDP5, Chr19: 4484708-4487708 |
| ARHGAP30 | Chr1: 159304884-159307884 | ARHGAP30, Chr1: 159304884-159307884 |
| SEBOX | Chr17: 23714892-23717892 | SEBOX, Chr17: 23714892-23717892 |
| ALS2CR11 | Chr2: 202190646-202193646 | ALS2CR11, Chr2: 202190646-202193646 |
| MOCS1 | Chr6: 40001933-40004933 | MOCS1, Chr6: 40001933-40004933 |
| OR10H1 | Chr19: 15778436-15781436 | OR10H1, Chr19: 15778436-15781436 |
| TREML2 | Chr6: 41275403-41278403 | TREML2, Chr6: 41275403-41278403 |
| DUPD1 | Chr10: 76486778-76489778 | DUPD1, Chr10: 76486778-76489778 |
| PAGE3 | Chr23: 55306504-55309504 | PAGE3, Chr23: 55306504-55309504 |
| WDR61 | Chr15: 76377495-76380495 | WDR61, Chr15: 76377495-76380495 |
| PLA2G3 | Chr22: 29864969-29867969 | PLA2G3, Chr22: 29864969-29867969 |
| PI16 | Chr6: 37028686-37031686 | PI16, Chr6: 37028686-37031686 |
| FAM129C | Chr19: 17493609-17496609 | FAM129C, Chr19: 17493609-17496609 |
| GOLGA2L1 | Chr12: 99089702-99092702 | GOLGA2L1, Chr12: 99089702-99092702 |
| RAB3IP | Chr12: 68457520-68460520 | RAB3IP, Chr12: 68457520-68460520 |
| SOST | Chr17: 39190182-39193182 | SOST, Chr17: 39190182-39193182 |
| SLC11A1 | Chr2: 218953495-218956495 | SLC11A1, Chr2: 218953495-218956495 |
| CLU | Chr8: 27523355-27526355 | CLU, Chr8: 27523355-27526355 |
| CACNA1S | Chr1: 199346817-199349817 | CACNA1S, Chr1: 199346817-199349817 |
| ZNF714 | Chr19: 21055310-21058310 | ZNF714, Chr19: 21055310-21058310 |
| NAPSA | Chr19: 55559243-55562243 | NAPSA, Chr19: 55559243-55562243 |
| SIGLEC6 | Chr19: 56725376-56728376 | SIGLEC6, Chr19: 56725376-56728376 |
| SDPR | Chr2: 192418726-192421726 | SDPR, Chr2: 192418726-192421726 |
| DARC | Chr1: 157440324-157443324 | DARC, Chr1: 157440324-157443324 |
| OR13J1 | Chr9: 35858898-35861898 | OR13J1, Chr9: 35858898-35861898 |
| P2RY6 | Chr11: 72651717-72654717 | P2RY6, Chr11: 72651717-72654717 |
| LRRC10 | Chr12: 68289709-68292709 | LRRC10, Chr12: 68289709-68292709 |
| FAM83A | Chr8: 124262432-124265432 | FAM83A, Chr8: 124262432-124265432 |
| TMPRSS6 | Chr22: 35828139-35831139 | TMPRSS6, Chr22: 35828139-35831139 |
| HMHB1 | Chr5: 143170418-143173418 | HMHB1, Chr5: 143170418-143173418 |
| SGCD | Chr5: 155684844-155687844 | SGCD, Chr5: 155684844-155687844 |
| ADPRHL1 | Chr13: 113149958-113152958 | ADPRHL1, Chr13: 113149958-113152958 |
| ASGR2 | Chr17: 6957352-6960352 | ASGR2, Chr17: 6957352-6960352 |
| TRIM15 | Chr6: 30237461-30240461 | TRIM15, Chr6: 30237461-30240461 |
| KRT74 | Chr12: 51252376-51255376 | KRT74, Chr12: 51252376-51255376 |
| TNFSF8 | Chr9: 116731091-116734091 | TNFSF8, Chr9: 116731091-116734091 |
| ARHGAP9 | Chr12: 56156495-56159495 | ARHGAP9, Chr12: 56156495-56159495 |
| FGF23 | Chr12: 4357655-4360655 | FGF23, Chr12: 4357655-4360655 |
| GABRP | Chr5: 170141842-170144842 | GABRP, Chr5: 170141842-170144842 |
| LPAR5 | Chr12: 6614048-6617048 | LPAR5, Chr12: 6614048-6617048 |
| TCF20 | Chr22: 40939889-40942889 | TCF20, Chr22: 40939889-40942889 |
| MLN | Chr6: 33878271-33881271 | MLN, Chr6: 33878271-33881271 |
| LOC389435 | Chr6: 153644086-153647086 | LOC389435, Chr6: 153644086-153647086 |
| IL25 | Chr14: 22910357-22913357 | IL25, Chr14: 22910357-22913357 |
| MAGEA12 | Chr23: 151652328-151655328 | MAGEA12, Chr23: 151652328-151655328 |
| SYNE2 | Chr14: 63749111-63752111 | SYNE2, Chr14: 63749111-63752111 |
| TSPAN18 | Chr11: 44883032-44886032 | TSPAN18, Chr11: 44883032-44886032 |
| TGM6 | Chr20: 2308053-2311053 | TGM6, Chr20: 2308053-2311053 |
| TGM2 | Chr20: 36225614-36228614 | TGM2, Chr20: 36225614-36228614 |
| EFHD1 | Chr2: 233205067-233208067 | EFHD1, Chr2: 233205067-233208067 |
| CATSPER1 | Chr11: 65549060-65552060 | CATSPER1, Chr11: 65549060-65552060 |
| UBA7 | Chr3: 49824895-49827895 | UBA7, Chr3: 49824895-49827895 |
| ADAMTS4 | Chr1: 159433969-159436969 | ADAMTS4, Chr1: 159433969-159436969 |
| MAGEA1 | Chr23: 152137810-152140810 | MAGEA1, Chr23: 152137810-152140810 |
| DCST2 | Chr1: 153271381-153274381 | DCST2, Chr1: 153271381-153274381 |
| VSIG8 | Chr1: 158097571-158100571 | VSIG8, Chr1: 158097571-158100571 |
| DNMT3B | Chr20: 30829818-30832818 | DNMT3B, Chr20: 30829818-30832818 |
| GDF5 | Chr20: 33487941-33490941 | GDF5, Chr20: 33487941-33490941 |
| OR6F1 | Chr1: 245941180-245944180 | OR6F1, Chr1: 245941180-245944180 |
| GPD1 | Chr12: 48782567-48785567 | GPD1, Chr12: 48782567-48785567 |
| NUDT17 | Chr1: 144299292-144302292 | NUDT17, Chr1: 144299292-144302292 |
| LRRC18 | Chr10: 49790786-49793786 | LRRC18, Chr10: 49790786-49793786 |
| BMF | Chr15: 38184431-38187431 | BMF, Chr15: 38184431-38187431 |
| LAT2 | Chr7: 73260522-73263522 | LAT2, Chr7: 73260522-73263522 |
| NCOR2 | Chr12: 123584602-123587602 | NCOR2, Chr12: 123584602-123587602 |
| CLK3 | Chr15: 72686265-72689265 | CLK3, Chr15: 72686265-72689265 |
| AQP8 | Chr16: 25134285-25137285 | AQP8, Chr16: 25134285-25137285 |
| IL1R1 | Chr2: 102135333-102138333 | IL1R1, Chr2: 102135333-102138333 |
| CDC27 | Chr17: 42620164-42623164 | CDC27, Chr17: 42620164-42623164 |
| PLXNB2 | Chr22: 49073836-49076836 | PLXNB2, Chr22: 49073836-49076836 |
| BCL9 | Chr1: 145478305-145481305 | BCL9, Chr1: 145478305-145481305 |
| KRT38 | Chr17: 36849622-36852622 | KRT38, Chr17: 36849622-36852622 |
| ABI3 | Chr17: 44641087-44644087 | ABI3, Chr17: 44641087-44644087 |
| GAGE2A | Chr23: 49102077-49105077 | GAGE2A, Chr23: 49102077-49105077 |
| GAGE2C | Chr23: 49102091-49105091 | GAGE2C, Chr23: 49102091-49105091 |
| GAGE4 | Chr23: 49102092-49105092 | GAGE4, Chr23: 49102092-49105092 |
| GAGE5 | Chr23: 49102100-49105100 | GAGE5, Chr23: 49102100-49105100 |
| KRT6A | Chr12: 51171948-51174948 | KRT6A, Chr12: 51171948-51174948 |
| GRB10 | Chr7: 50738992-50741992 | GRB10, Chr7: 50738992-50741992 |
| VSTM1 | Chr19: 59257519-59260519 | VSTM1, Chr19: 59257519-59260519 |
| GAGE8 | Chr23: 49102102-49105102 | GAGE8, Chr23: 49102102-49105102 |
| GAGE12I | Chr23: 49102120-49105120 | GAGE12I, Chr23: 49102120-49105120 |
| GAGE7 | Chr23: 49102121-49105121 | GAGE7, Chr23: 49102121-49105121 |
| TREML1 | Chr6: 41228548-41231548 | TREML1, Chr6: 41228548-41231548 |
| KRT6B | Chr12: 51130677-51133677 | KRT6B, Chr12: 51130677-51133677 |
| C1QTNF6 | Chr22: 35912776-35915776 | C1QTNF6, Chr22: 35912776-35915776 |
| JPH2 | Chr20: 42248132-42251132 | JPH2, Chr20: 42248132-42251132 |
| CHP2 | Chr16: 23671948-23674948 | CHP2, Chr16: 23671948-23674948 |
| C20orf165 | Chr20: 43948145-43951145 | C20orf165, Chr20: 43948145-43951145 |
| CYP2A6 | Chr19: 46046692-46049692 | CYP2A6, Chr19: 46046692-46049692 |
| APBA2 | Chr15: 26999644-27002644 | APBA2, Chr15: 26999644-27002644 |
| NPPB | Chr1: 11840079-11843079 | NPPB, Chr1: 11840079-11843079 |
| C9orf79 | Chr9: 89686091-89689091 | C9orf79, Chr9: 89686091-89689091 |
| CYP8B1 | Chr3: 42891137-42894137 | CYP8B1, Chr3: 42891137-42894137 |
| APOB48R | Chr16: 28411993-28414993 | APOB48R, Chr16: 28411993-28414993 |
| IL4I1 | Chr19: 55090459-55093459 | IL4I1, Chr19: 55090459-55093459 |
| SCGB1C1 | Chr11: 181579-184579 | SCGB1C1, Chr11: 181579-184579 |
| SLC26A9 | Chr1: 204177711-204180711 | SLC26A9, Chr1: 204177711-204180711 |
| CCDC89 | Chr11: 85073468-85076468 | CCDC89, Chr11: 85073468-85076468 |
| GPHA2 | Chr11: 64458436-64461436 | GPHA2, Chr11: 64458436-64461436 |
| PKLR | Chr1: 153535916-153538916 | PKLR, Chr1: 153535916-153538916 |
| ASTL | Chr2: 96166402-96169402 | ASTL, Chr2: 96166402-96169402 |
| UPK3A | Chr22: 44058052-44061052 | UPK3A, Chr22: 44058052-44061052 |
| MFRP | Chr11: 118721080-118724080 | MFRP, Chr11: 118721080-118724080 |
| BTNL2 | Chr6: 32481378-32484378 | BTNL2, Chr6: 32481378-32484378 |
| GDF10 | Chr10: 48057672-48060672 | GDF10, Chr10: 48057672-48060672 |
| CXorf48 | Chr23: 134131917-134134917 | CXorf48, Chr23: 134131917-134134917 |
| SIGLEC12 | Chr19: 56693959-56696959 | SIGLEC12, Chr19: 56693959-56696959 |
| TMC2 | Chr20: 2463752-2466752 | TMC2, Chr20: 2463752-2466752 |
| C6orf15 | Chr6: 31186811-31189811 | C6orf15, Chr6: 31186811-31189811 |
| RASGRP4 | Chr19: 43607285-43610285 | RASGRP4, Chr19: 43607285-43610285 |
| CNR1 | Chr6: 88910275-88913275 | CNR1, Chr6: 88910275-88913275 |
| C10orf62 | Chr10: 99337973-99340973 | C10orf62, Chr10: 99337973-99340973 |
| LOC348174 | Chr16: 68540810-68543810 | LOC348174, Chr16: 68540810-68543810 |
| PRDM11 | Chr11: 45070639-45073639 | PRDM11, Chr11: 45070639-45073639 |
| LOC402573 | Chr7: 99898330-99901330 | LOC402573, Chr7: 99898330-99901330 |
| MICALCL | Chr11: 12263522-12266522 | MICALCL, Chr11: 12263522-12266522 |
| ABLIM1 | Chr10: 116432904-116435904 | ABLIM1, Chr10: 116432904-116435904 |
| KRT4 | Chr12: 51493102-51496102 | KRT4, Chr12: 51493102-51496102 |
| DPP6 | Chr7: 153631779-153634779 | DPP6, Chr7: 153631779-153634779 |
| TSSK4 | Chr14: 23743301-23746301 | TSSK4, Chr14: 23743301-23746301 |
| SPRR2A | Chr1: 151295112-151298112 | SPRR2A, Chr1: 151295112-151298112 |
| MVP | Chr16: 29737787-29740787 | MVP, Chr16: 29737787-29740787 |
| MVP | Chr16: 29737788-29740788 | MVP, Chr16: 29737788-29740788 |
| SPRR2D | Chr1: 151278718-151281718 | SPRR2D, Chr1: 151278718-151281718 |
| SPRR2E | Chr1: 151332125-151335125 | SPRR2E, Chr1: 151332125-151335125 |
| MB | Chr22: 34347847-34350847 | MB, Chr22: 34347847-34350847 |
| DNAJC5B | Chr8: 67094844-67097844 | DNAJC5B, Chr8: 67094844-67097844 |
| RHOXF2B | Chr23: 119174994-119177994 | RHOXF2B, Chr23: 119174994-119177994 |
| SPRR2F | Chr1: 151351113-151354113 | SPRR2F, Chr1: 151351113-151354113 |
| RASGEF1A | Chr10: 43080873-43083873 | RASGEF1A, Chr10: 43080873-43083873 |
| S100A8 | Chr1: 151628673-151631673 | S100A8, Chr1: 151628673-151631673 |
| DARC | Chr1: 157439633-157442633 | DARC, Chr1: 157439633-157442633 |
| S100A3 | Chr1: 151786858-151789858 | S100A3, Chr1: 151786858-151789858 |
| CORO2A | Chr9: 99973495-99976495 | CORO2A, Chr9: 99973495-99976495 |
| KIR3DL1 | Chr19: 60018204-60021204 | KIR3DL1, Chr19: 60018204-60021204 |
| SLC26A11 | Chr17: 75807331-75810331 | SLC26A11, Chr17: 75807331-75810331 |
| PKD1L1 | Chr7: 47953062-47956062 | PKD1L1, Chr7: 47953062-47956062 |
| IQSEC2 | Chr23: 53326021-53329021 | IQSEC2, Chr23: 53326021-53329021 |
| SPTBN2 | Chr11: 66243946-66246946 | SPTBN2, Chr11: 66243946-66246946 |
| SIGLEC14 | Chr19: 56840444-56843444 | SIGLEC14, Chr19: 56840444-56843444 |
| CDH16 | Chr16: 65508767-65511767 | CDH16, Chr16: 65508767-65511767 |
| GH2 | Chr17: 59311455-59314455 | GH2, Chr17: 59311455-59314455 |
| SFTPC | Chr8: 22073628-22076628 | SFTPC, Chr8: 22073628-22076628 |
| DPH2 | Chr1: 44206739-44209739 | DPH2, Chr1: 44206739-44209739 |
| ATG9B | Chr7: 150351019-150354019 | ATG9B, Chr7: 150351019-150354019 |
| FAM14A | Chr14: 93664210-93667210 | FAM14A, Chr14: 93664210-93667210 |
| CPNE1 | Chr20: 33703745-33706745 | CPNE1, Chr20: 33703745-33706745 |
| SLC25A11 | Chr17: 4782563-4785563 | SLC25A11, Chr17: 4782563-4785563 |
| CUL7 | Chr6: 43128132-43131132 | CUL7, Chr6: 43128132-43131132 |
| TMEM173 | Chr5: 138840976-138843976 | TMEM173, Chr5: 138840976-138843976 |
| C3 | Chr19: 6670162-6673162 | C3, Chr19: 6670162-6673162 |
| PKLR | Chr1: 153536349-153539349 | PKLR, Chr1: 153536349-153539349 |
| TSKS | Chr19: 54956827-54959827 | TSKS, Chr19: 54956827-54959827 |
| ABCC12 | Chr16: 46736682-46739682 | ABCC12, Chr16: 46736682-46739682 |
| SPRR1A | Chr1: 151221680-151224680 | SPRR1A, Chr1: 151221680-151224680 |
| CYP2A7 | Chr19: 46078997-46081997 | CYP2A7, Chr19: 46078997-46081997 |
| SPRR1B | Chr1: 151268802-151271802 | SPRR1B, Chr1: 151268802-151271802 |
| RGL4 | Chr22: 22361547-22364547 | RGL4, Chr22: 22361547-22364547 |
| KCNJ8 | Chr12: 21817514-21820514 | KCNJ8, Chr12: 21817514-21820514 |
| SLA | Chr8: 134140285-134143285 | SLA, Chr8: 134140285-134143285 |
| MAP1LC3A | Chr20: 32596852-32599852 | MAP1LC3A, Chr20: 32596852-32599852 |
| MC2R | Chr18: 13904035-13907035 | MC2R, Chr18: 13904035-13907035 |
| S100Z | Chr5: 76180081-76183081 | S100Z, Chr5: 76180081-76183081 |
| RAB11FIP1 | Chr8: 37848454-37851454 | RAB11FIP1, Chr8: 37848454-37851454 |
| LOC340286 | Chr7: 38691662-38694662 | LOC340286, Chr7: 38691662-38694662 |
| BDKRB2 | Chr14: 95739387-95742387 | BDKRB2, Chr14: 95739387-95742387 |
| CA5A | Chr16: 86526113-86529113 | CA5A, Chr16: 86526113-86529113 |
| FLT3LG | Chr19: 54667797-54670797 | FLT3LG, Chr19: 54667797-54670797 |
| FLJ42957 | Chr12: 115454109-115457109 | FLJ42957, Chr12: 115454109-115457109 |
| TRIM63 | Chr1: 26265208-26268208 | TRIM63, Chr1: 26265208-26268208 |
| ZBED2 | Chr3: 112795356-112798356 | ZBED2, Chr3: 112795356-112798356 |
| CCDC99 | Chr5: 168941715-168944715 | CCDC99, Chr5: 168941715-168944715 |
| PROC | Chr2: 127890986-127893986 | PROC, Chr2: 127890986-127893986 |
| NME4 | Chr16: 385692-388692 | NME4, Chr16: 385692-388692 |
| TMEM208 | Chr16: 65817016-65820016 | TMEM208, Chr16: 65817016-65820016 |
| SLC23A3 | Chr2: 219741530-219744530 | SLC23A3, Chr2: 219741530-219744530 |
| ODF2L | Chr1: 86633091-86636091 | ODF2L, Chr1: 86633091-86636091 |
| CFB | Chr6: 32020199-32023199 | CFB, Chr6: 32020199-32023199 |
| LELP1 | Chr1: 151441042-151444042 | LELP1, Chr1: 151441042-151444042 |
| GLI2 | Chr2: 121269836-121272836 | GLI2, Chr2: 121269836-121272836 |
| TNF | Chr6: 31649828-31652828 | TNF, Chr6: 31649828-31652828 |
| RARRES3 | Chr11: 63059348-63062348 | RARRES3, Chr11: 63059348-63062348 |
| IL18BP | Chr11: 71386256-71389256 | IL18BP, Chr11: 71386256-71389256 |
| ACBD4 | Chr17: 40567079-40570079 | ACBD4, Chr17: 40567079-40570079 |
| PLOD3 | Chr7: 100646231-100649231 | PLOD3, Chr7: 100646231-100649231 |
| KCND1 | Chr23: 48711695-48714695 | KCND1, Chr23: 48711695-48714695 |
| SLC5A2 | Chr16: 31400439-31403439 | SLC5A2, Chr16: 31400439-31403439 |
| MT1X | Chr16: 55272382-55275382 | MT1X, Chr16: 55272382-55275382 |
| SERPINA10 | Chr14: 93827614-93830614 | SERPINA10, Chr14: 93827614-93830614 |
| LILRA3 | Chr19: 59494550-59497550 | LILRA3, Chr19: 59494550-59497550 |
| PCK1 | Chr20: 55568042-55571042 | PCK1, Chr20: 55568042-55571042 |
| CCL22 | Chr16: 55948718-55951718 | CCL22, Chr16: 55948718-55951718 |
| IL18BP | Chr11: 71386105-71389105 | IL18BP, Chr11: 71386105-71389105 |
| NPFF | Chr12: 52186189-52189189 | NPFF, Chr12: 52186189-52189189 |
| ETFB | Chr19: 56548408-56551408 | ETFB, Chr19: 56548408-56551408 |
| PDZD3 | Chr11: 118559904-118562904 | PDZD3, Chr11: 118559904-118562904 |
| PIK3R6 | Chr17: 8710219-8713219 | PIK3R6, Chr17: 8710219-8713219 |
| CCDC80 | Chr3: 113841167-113844167 | CCDC80, Chr3: 113841167-113844167 |
| TCP10 | Chr6: 167716488-167719488 | TCP10, Chr6: 167716488-167719488 |
| SPATA19 | Chr11: 133219102-133222102 | SPATA19, Chr11: 133219102-133222102 |
| CHI3L1 | Chr1: 201421045-201424045 | CHI3L1, Chr1: 201421045-201424045 |
| ZNF692 | Chr1: 247118394-247121394 | ZNF692, Chr1: 247118394-247121394 |
| FOXG1 | Chr14: 28304537-28307537 | FOXG1, Chr14: 28304537-28307537 |
| ICAM4 | Chr19: 10257149-10260149 | ICAM4, Chr19: 10257149-10260149 |
| IRS1 | Chr2: 227370250-227373250 | IRS1, Chr2: 227370250-227373250 |
| LMX1B | Chr9: 128415068-128418068 | LMX1B, Chr9: 128415068-128418068 |
| CELSR1 | Chr22: 45310231-45313231 | CELSR1, Chr22: 45310231-45313231 |
| NAT8L | Chr4: 2029884-2032884 | NAT8L, Chr4: 2029884-2032884 |
| FZD7 | Chr2: 202606054-202609054 | FZD7, Chr2: 202606054-202609054 |
| PPP1R3D | Chr20: 57947247-57950247 | PPP1R3D, Chr20: 57947247-57950247 |
| PDGFA | Chr7: 524507-527507 | PDGFA, Chr7: 524507-527507 |
| HIC1 | Chr17: 1903642-1906642 | HIC1, Chr17: 1903642-1906642 |
| HIC1 | Chr17: 1904853-1907853 | HIC1, Chr17: 1904853-1907853 |
| C20orf177 | Chr20: 57947450-57950450 | C20orf177, Chr20: 57947450-57950450 |
| 11-Sep | Chr4: 78088418-78091418 | 40797, Chr4: 78088418-78091418 |
| PTBP1 | Chr19: 746910-749910 | PTBP1, Chr19: 746910-749910 |
| FZD5 | Chr2: 208340888-208343888 | FZD5, Chr2: 208340888-208343888 |
| U2AF2 | Chr19: 60855727-60858727 | U2AF2, Chr19: 60855727-60858727 |
| MESDC1 | Chr15: 79079527-79082527 | MESDC1, Chr15: 79079527-79082527 |
| PXMP2 | Chr12: 131772764-131775764 | PXMP2, Chr12: 131772764-131775764 |
| NME3 | Chr16: 1760211-1763211 | NME3, Chr16: 1760211-1763211 |
| EME2 | Chr16: 1761729-1764729 | EME2, Chr16: 1761729-1764729 |
| MRPS34 | Chr16: 1761641-1764641 | MRPS34, Chr16: 1761641-1764641 |
| ADRA1D | Chr20: 4176159-4179159 | ADRA1D, Chr20: 4176159-4179159 |
| ZCCHC14 | Chr16: 86081461-86084461 | ZCCHC14, Chr16: 86081461-86084461 |
| EGLN2 | Chr19: 45996520-45999520 | EGLN2, Chr19: 45996520-45999520 |
| ABCD1 | Chr23: 152642029-152645029 | ABCD1, Chr23: 152642029-152645029 |
| ADCY5 | Chr3: 124648582-124651582 | ADCY5, Chr3: 124648582-124651582 |
| NOTUM | Chr17: 77510853-77513853 | NOTUM, Chr17: 77510853-77513853 |
| KCNF1 | Chr2: 10968013-10971013 | KCNF1, Chr2: 10968013-10971013 |
| KANK2 | Chr19: 11165996-11168996 | KANK2, Chr19: 11165996-11168996 |
| GSC | Chr14: 94304752-94307752 | GSC, Chr14: 94304752-94307752 |
| DIO3 | Chr14: 101095940-101098940 | DIO3, Chr14: 101095940-101098940 |
| IRX4 | Chr5: 1934380-1937380 | IRX4, Chr5: 1934380-1937380 |
| BRSK2 | Chr11: 1366204-1369204 | BRSK2, Chr11: 1366204-1369204 |
| KLHL17 | Chr1: 884329-887329 | KLHL17, Chr1: 884329-887329 |
| MED16 | Chr19: 842718-845718 | MED16, Chr19: 842718-845718 |
| ACHE | Chr7: 100329977-100332977 | ACHE, Chr7: 100329977-100332977 |
| ENPP7 | Chr17: 75317976-75320976 | ENPP7, Chr17: 75317976-75320976 |
| CREBBP | Chr16: 3868622-3871622 | CREBBP, Chr16: 3868622-3871622 |
| MEX3D | Chr19: 1517557-1520557 | MEX3D, Chr19: 1517557-1520557 |
| GZF1 | Chr20: 23291520-23294520 | GZF1, Chr20: 23291520-23294520 |
| LRFN4 | Chr11: 66380048-66383048 | LRFN4, Chr11: 66380048-66383048 |
| HRAS | Chr11: 524050-527050 | HRAS, Chr11: 524050-527050 |
| PUSL1 | Chr1: 1232356-1235356 | PUSL1, Chr1: 1232356-1235356 |
| INTS1 | Chr7: 1509044-1512044 | INTS1, Chr7: 1509044-1512044 |
| KIAA0182 | Chr16: 84202924-84205924 | KIAA0182, Chr16: 84202924-84205924 |
| 10-Sep | Chr2: 109727572-109730572 | 40796, Chr2: 109727572-109730572 |
| BCAP31 | Chr23: 152641581-152644581 | BCAP31, Chr23: 152641581-152644581 |
| IRF2BP1 | Chr19: 51079716-51082716 | IRF2BP1, Chr19: 51079716-51082716 |
| SPHK1 | Chr17: 71890796-71893796 | SPHK1, Chr17: 71890796-71893796 |
| SPHK1 | Chr17: 71890825-71893825 | SPHK1, Chr17: 71890825-71893825 |
| APC2 | Chr19: 1399647-1402647 | APC2, Chr19: 1399647-1402647 |
| TTLL10 | Chr1: 1097648-1100648 | TTLL10, Chr1: 1097648-1100648 |
| PCGF3 | Chr4: 688072-691072 | PCGF3, Chr4: 688072-691072 |
| ATHL1 | Chr11: 277637-280637 | ATHL1, Chr11: 277637-280637 |
| ISG15 | Chr1: 937209-940209 | ISG15, Chr1: 937209-940209 |
| FOXF1 | Chr16: 85100133-85103133 | FOXF1, Chr16: 85100133-85103133 |
| RBM35B | Chr16: 66826137-66829137 | RBM35B, Chr16: 66826137-66829137 |
| NSD1 | Chr5: 176491938-176494938 | NSD1, Chr5: 176491938-176494938 |
| ZNF710 | Chr15: 88344255-88347255 | ZNF710, Chr15: 88344255-88347255 |
| ENOPH1 | Chr4: 83569249-83572249 | ENOPH1, Chr4: 83569249-83572249 |
| PAX9 | Chr14: 36195032-36198032 | PAX9, Chr14: 36195032-36198032 |
| SH3RF3 | Chr2: 109110928-109113928 | SH3RF3, Chr2: 109110928-109113928 |
| HAND2 | Chr4: 174686453-174689453 | HAND2, Chr4: 174686453-174689453 |
| KCNAB3 | Chr17: 7771978-7774978 | KCNAB3, Chr17: 7771978-7774978 |
| GATA3 | Chr10: 8135172-8138172 | GATA3, Chr10: 8135172-8138172 |
| IRF7 | Chr11: 604499-607499 | IRF7, Chr11: 604499-607499 |
| TUBB2B | Chr6: 3171370-3174370 | TUBB2B, Chr6: 3171370-3174370 |
| BMP8A | Chr1: 39728404-39731404 | BMP8A, Chr1: 39728404-39731404 |
| SOX8 | Chr16: 970308-973308 | SOX8, Chr16: 970308-973308 |
| NKD2 | Chr5: 1060667-1063667 | NKD2, Chr5: 1060667-1063667 |
| FAM100A | Chr16: 4603428-4606428 | FAM100A, Chr16: 4603428-4606428 |
| JSRP1 | Chr19: 2204844-2207844 | JSRP1, Chr19: 2204844-2207844 |
| OXT | Chr20: 2998765-3001765 | OXT, Chr20: 2998765-3001765 |
| TBKBP1 | Chr17: 43126128-43129128 | TBKBP1, Chr17: 43126128-43129128 |
| RFNG | Chr17: 77601439-77604439 | RFNG, Chr17: 77601439-77604439 |
| TYMP | Chr22: 49313880-49316880 | TYMP, Chr22: 49313880-49316880 |
| C5orf38 | Chr5: 2803761-2806761 | C5orf38, Chr5: 2803761-2806761 |
| GNAL | Chr18: 11740685-11743685 | GNAL, Chr18: 11740685-11743685 |
| RRP1B | Chr21: 43902359-43905359 | RRP1B, Chr21: 43902359-43905359 |
| SDF4 | Chr1: 1155810-1158810 | SDF4, Chr1: 1155810-1158810 |
| MAP4K1 | Chr19: 43798983-43801983 | MAP4K1, Chr19: 43798983-43801983 |
| EIF3K | Chr19: 43800061-43803061 | EIF3K, Chr19: 43800061-43803061 |
| VAX1 | Chr10: 118886302-118889302 | VAX1, Chr10: 118886302-118889302 |
| KIAA1542 | Chr11: 564985-567985 | KIAA1542, Chr11: 564985-567985 |
| SNURF | Chr15: 22749662-22752662 | SNURF, Chr15: 22749662-22752662 |
| IRF7 | Chr11: 604228-607228 | IRF7, Chr11: 604228-607228 |
| ADAMTS16 | Chr5: 5191942-5194942 | ADAMTS16, Chr5: 5191942-5194942 |
| BRCA1 | Chr17: 38529494-38532494 | BRCA1, Chr17: 38529494-38532494 |
| RXRA | Chr9: 136356730-136359730 | RXRA, Chr9: 136356730-136359730 |
| ADCY2 | Chr5: 7447842-7450842 | ADCY2, Chr5: 7447842-7450842 |
| NDUFA13 | Chr19: 19486039-19489039 | NDUFA13, Chr19: 19486039-19489039 |
| DRD4 | Chr11: 625804-628804 | DRD4, Chr11: 625804-628804 |
| TYMP | Chr22: 49313624-49316624 | TYMP, Chr22: 49313624-49316624 |
| MYO1C | Chr17: 1334301-1337301 | MYO1C, Chr17: 1334301-1337301 |
| SSBP4 | Chr19: 18389635-18392635 | SSBP4, Chr19: 18389635-18392635 |
| LRPAP1 | Chr4: 3502442-3505442 | LRPAP1, Chr4: 3502442-3505442 |
| C19orf23 | Chr19: 1219759-1222759 | C19orf23, Chr19: 1219759-1222759 |
| TNFRSF18 | Chr1: 1130452-1133452 | TNFRSF18, Chr1: 1130452-1133452 |
| PHPT1 | Chr9: 138861866-138864866 | PHPT1, Chr9: 138861866-138864866 |
| BOLL | Chr2: 198356819-198359819 | BOLL, Chr2: 198356819-198359819 |
| SIVA1 | Chr14: 104289014-104292014 | SIVA1, Chr14: 104289014-104292014 |
| FAM53A | Chr4: 1654016-1657016 | FAM53A, Chr4: 1654016-1657016 |
| TP73 | Chr1: 3557488-3560488 | TP73, Chr1: 3557488-3560488 |
| EIF4E3 | Chr3: 71883965-71886965 | EIF4E3, Chr3: 71883965-71886965 |
| AADAT | Chr4: 171246258-171249258 | AADAT, Chr4: 171246258-171249258 |
| TNFRSF10B | Chr8: 22981137-22984137 | TNFRSF10B, Chr8: 22981137-22984137 |
| DUSP9 | Chr23: 152559590-152562590 | DUSP9, Chr23: 152559590-152562590 |
| SCT | Chr11: 615673-618673 | SCT, Chr11: 615673-618673 |
| AGPAT9 | Chr4: 84675176-84678176 | AGPAT9, Chr4: 84675176-84678176 |
| SNRPN | Chr15: 22749727-22752727 | SNRPN, Chr15: 22749727-22752727 |
| IRF5 | Chr7: 128363729-128366729 | IRF5, Chr7: 128363729-128366729 |
| PWWP2B | Chr10: 134059191-134062191 | PWWP2B, Chr10: 134059191-134062191 |
| PCDH17 | Chr13: 57102289-57105289 | PCDH17, Chr13: 57102289-57105289 |
| RAB1B | Chr11: 65791131-65794131 | RAB1B, Chr11: 65791131-65794131 |
| NAP1L5 | Chr4: 89836503-89839503 | NAP1L5, Chr4: 89836503-89839503 |
| HMG20B | Chr19: 3522442-3525442 | HMG20B, Chr19: 3522442-3525442 |
| GDNF | Chr5: 37869850-37872850 | GDNF, Chr5: 37869850-37872850 |
| FAM71E1 | Chr19: 55670315-55673315 | FAM71E1, Chr19: 55670315-55673315 |
| MGC70857 | Chr8: 145723766-145726766 | MGC70857, Chr8: 145723766-145726766 |
| KCNIP3 | Chr2: 95374994-95377994 | KCNIP3, Chr2: 95374994-95377994 |
| NPB | Chr17: 77451945-77454945 | NPB, Chr17: 77451945-77454945 |
| NXPH3 | Chr17: 45006977-45009977 | NXPH3, Chr17: 45006977-45009977 |
| SAMD11 | Chr1: 849483-852483 | SAMD11, Chr1: 849483-852483 |
| WDR8 | Chr1: 3554997-3557997 | WDR8, Chr1: 3554997-3557997 |
| PLCL2 | Chr3: 16900172-16903172 | PLCL2, Chr3: 16900172-16903172 |
| BRCA1 | Chr17: 38529157-38532157 | BRCA1, Chr17: 38529157-38532157 |
| LRRC24 | Chr8: 145721716-145724716 | LRRC24, Chr8: 145721716-145724716 |
| PAPD5 | Chr16: 48744068-48747068 | PAPD5, Chr16: 48744068-48747068 |
| HTR6 | Chr1: 19862866-19865866 | HTR6, Chr1: 19862866-19865866 |
| NT5DC2 | Chr3: 52542610-52545610 | NT5DC2, Chr3: 52542610-52545610 |
| CTRB2 | Chr16: 73797073-73800073 | CTRB2, Chr16: 73797073-73800073 |
| GRIK5 | Chr19: 47260297-47263297 | GRIK5, Chr19: 47260297-47263297 |
| SSTR5 | Chr16: 1067369-1070369 | SSTR5, Chr16: 1067369-1070369 |
| TPPP3 | Chr16: 65983422-65986422 | TPPP3, Chr16: 65983422-65986422 |
| CHST3 | Chr10: 73392625-73395625 | CHST3, Chr10: 73392625-73395625 |
| LOC388931 | Chr2: 24084956-24087956 | LOC388931, Chr2: 24084956-24087956 |
| MT1B | Chr16: 55241811-55244811 | MT1B, Chr16: 55241811-55244811 |
| TBX1 | Chr22: 18122725-18125725 | TBX1, Chr22: 18122725-18125725 |
| ACCN3 | Chr7: 150375037-150378037 | ACCN3, Chr7: 150375037-150378037 |
| C11orf35 | Chr11: 549279-552279 | C11orf35, Chr11: 549279-552279 |
| LRRC56 | Chr11: 526026-529026 | LRRC56, Chr11: 526026-529026 |
| SLC25A10 | Chr17: 77288275-77291275 | SLC25A10, Chr17: 77288275-77291275 |
| IRF5 | Chr7: 128364006-128367006 | IRF5, Chr7: 128364006-128367006 |
| SP5 | Chr2: 171278606-171281606 | SP5, Chr2: 171278606-171281606 |
| HORMAD2 | Chr22: 28804952-28807952 | HORMAD2, Chr22: 28804952-28807952 |
| PSG3 | Chr19: 47935008-47938008 | PSG3, Chr19: 47935008-47938008 |
| UHRF1 | Chr19: 4860065-4863065 | UHRF1, Chr19: 4860065-4863065 |
| HOXB5 | Chr17: 44024602-44027602 | HOXB5, Chr17: 44024602-44027602 |
| DMRT2 | Chr9: 1040113-1043113 | DMRT2, Chr9: 1040113-1043113 |
| MUC1 | Chr1: 153427824-153430824 | MUC1, Chr1: 153427824-153430824 |
| CCDC22 | Chr23: 48977384-48980384 | CCDC22, Chr23: 48977384-48980384 |
| TBX4 | Chr17: 56887088-56890088 | TBX4, Chr17: 56887088-56890088 |
| CLEC2L | Chr7: 138857713-138860713 | CLEC2L, Chr7: 138857713-138860713 |
| KCNB1 | Chr20: 47531088-47534088 | KCNB1, Chr20: 47531088-47534088 |
| SATB2 | Chr2: 200032000-200035000 | SATB2, Chr2: 200032000-200035000 |
| GNAS | Chr20: 56859930-56862930 | GNAS, Chr20: 56859930-56862930 |
| LOC199800 | Chr19: 54882253-54885253 | LOC199800, Chr19: 54882253-54885253 |
| BRF1 | Chr14: 104783767-104786767 | BRF1, Chr14: 104783767-104786767 |
| LOXL3 | Chr2: 74633070-74636070 | LOXL3, Chr2: 74633070-74636070 |
| BAIAP3 | Chr16: 1323163-1326163 | BAIAP3, Chr16: 1323163-1326163 |
| SYCP2 | Chr20: 57939104-57942104 | SYCP2, Chr20: 57939104-57942104 |
| JARID2 | Chr6: 15353005-15356005 | JARID2, Chr6: 15353005-15356005 |
| KIF2C | Chr1: 44976576-44979576 | KIF2C, Chr1: 44976576-44979576 |
| DKKL1 | Chr19: 54557353-54560353 | DKKL1, Chr19: 54557353-54560353 |
| ZNF74 | Chr22: 19076979-19079979 | ZNF74, Chr22: 19076979-19079979 |
| RNF151 | Chr16: 1955375-1958375 | RNF151, Chr16: 1955375-1958375 |
| ROM1 | Chr11: 62135698-62138698 | ROM1, Chr11: 62135698-62138698 |
| CCDC115 | Chr2: 130814892-130817892 | CCDC115, Chr2: 130814892-130817892 |
| SAMD10 | Chr20: 62079939-62082939 | SAMD10, Chr20: 62079939-62082939 |
| MFSD7 | Chr4: 671473-674473 | MFSD7, Chr4: 671473-674473 |
| C8G | Chr9: 138958018-138961018 | C8G, Chr9: 138958018-138961018 |
| BRD9 | Chr5: 942903-945903 | BRD9, Chr5: 942903-945903 |
| KAT2A | Chr17: 37525408-37528408 | KAT2A, Chr17: 37525408-37528408 |
| TOX2 | Chr20: 41976695-41979695 | TOX2, Chr20: 41976695-41979695 |
| HSPB9 | Chr17: 37526894-37529894 | HSPB9, Chr17: 37526894-37529894 |
| GJB6 | Chr13: 19701620-19704620 | GJB6, Chr13: 19701620-19704620 |
| OPCML | Chr11: 132316747-132319747 | OPCML, Chr11: 132316747-132319747 |
| C1QTNF1 | Chr17: 74530345-74533345 | C1QTNF1, Chr17: 74530345-74533345 |
| DPEP3 | Chr16: 66570453-66573453 | DPEP3, Chr16: 66570453-66573453 |
| GDNF | Chr5: 37869155-37872155 | GDNF, Chr5: 37869155-37872155 |
| NR4A3 | Chr9: 101627329-101630329 | NR4A3, Chr9: 101627329-101630329 |
| TPSAB1 | Chr16: 1229178-1232178 | TPSAB1, Chr16: 1229178-1232178 |
| LOC100129534 | Chr1: 2271974-2274974 | LOC100129534, Chr1: 2271974-2274974 |
| TRIML2 | Chr4: 189261902-189264902 | TRIML2, Chr4: 189261902-189264902 |
| MC5R | Chr18: 13814264-13817264 | MC5R, Chr18: 13814264-13817264 |
| CAMKK1 | Chr17: 3739286-3742286 | CAMKK1, Chr17: 3739286-3742286 |
| LOC126147 | Chr19: 53866576-53869576 | LOC126147, Chr19: 53866576-53869576 |
| CD7 | Chr17: 77867269-77870269 | CD7, Chr17: 77867269-77870269 |
| BHLHB8 | Chr7: 97678001-97681001 | BHLHB8, Chr7: 97678001-97681001 |
| GSDMDC1 | Chr8: 144710134-144713134 | GSDMDC1, Chr8: 144710134-144713134 |
| GRIFIN | Chr7: 2522410-2525410 | GRIFIN, Chr7: 2522410-2525410 |
| PLEKHG4B | Chr5: 191872-194872 | PLEKHG4B, Chr5: 191872-194872 |
| LOC554235 | Chr19: 55707454-55710454 | LOC554235, Chr19: 55707454-55710454 |
| HGC6.3 | Chr6: 168118968-168121968 | HGC6.3, Chr6: 168118968-168121968 |
| PDGFB | Chr22: 37965365-37968365 | PDGFB, Chr22: 37965365-37968365 |
| PLEC1 | Chr8: 145120031-145123031 | PLEC1, Chr8: 145120031-145123031 |
| LRRC55 | Chr11: 56704296-56707296 | LRRC55, Chr11: 56704296-56707296 |
| CGB2 | Chr19: 54225441-54228441 | CGB2, Chr19: 54225441-54228441 |
| GARNL4 | Chr17: 2644981-2647981 | GARNL4, Chr17: 2644981-2647981 |
| RFPL3 | Chr22: 31079371-31082371 | RFPL3, Chr22: 31079371-31082371 |
| TAS1R3 | Chr1: 1255088-1258088 | TAS1R3, Chr1: 1255088-1258088 |
| HIF3A | Chr19: 51491998-51494998 | HIF3A, Chr19: 51491998-51494998 |
| C10orf92 | Chr10: 134519726-134522726 | C10orf92, Chr10: 134519726-134522726 |
| MGC24975 | Chr19: 5734110-5737110 | MGC24975, Chr19: 5734110-5737110 |
| ERAS | Chr23: 48570726-48573726 | ERAS, Chr23: 48570726-48573726 |
| NSD1 | Chr5: 176491185-176494185 | NSD1, Chr5: 176491185-176494185 |
| CELSR3 | Chr3: 48673852-48676852 | CELSR3, Chr3: 48673852-48676852 |
| CIRBP | Chr19: 1218766-1221766 | CIRBP, Chr19: 1218766-1221766 |
| DLX4 | Chr17: 45403628-45406628 | DLX4, Chr17: 45403628-45406628 |
| ZNF276 | Chr16: 88313952-88316952 | ZNF276, Chr16: 88313952-88316952 |
| PQLC1 | Chr18: 75811105-75814105 | PQLC1, Chr18: 75811105-75814105 |
| SLC6A19 | Chr5: 1253209-1256209 | SLC6A19, Chr5: 1253209-1256209 |
| SHANK3 | Chr22: 49458435-49461435 | SHANK3, Chr22: 49458435-49461435 |
| PTDSS2 | Chr11: 438779-441779 | PTDSS2, Chr11: 438779-441779 |
| SERTAD3 | Chr19: 45638845-45641845 | SERTAD3, Chr19: 45638845-45641845 |
| JPH3 | Chr16: 86192499-86195499 | JPH3, Chr16: 86192499-86195499 |
| C20orf149 | Chr20: 61621076-61624076 | C20orf149, Chr20: 61621076-61624076 |
| LIMD2 | Chr17: 59129751-59132751 | LIMD2, Chr17: 59129751-59132751 |
| AADAT | Chr4: 171246447-171249447 | AADAT, Chr4: 171246447-171249447 |
| LFNG | Chr7: 2524504-2527504 | LFNG, Chr7: 2524504-2527504 |
| ADRA2B | Chr2: 96144115-96147115 | ADRA2B, Chr2: 96144115-96147115 |
| CYP26C1 | Chr10: 94809510-94812510 | CYP26C1, Chr10: 94809510-94812510 |
| SEC16A | Chr9: 138495828-138498828 | SEC16A, Chr9: 138495828-138498828 |
| TTLL10 | Chr1: 1103439-1106439 | TTLL10, Chr1: 1103439-1106439 |
| ACOT4 | Chr14: 73126662-73129662 | ACOT4, Chr14: 73126662-73129662 |
| ARHGEF15 | Chr17: 8152814-8155814 | ARHGEF15, Chr17: 8152814-8155814 |
| NOC4L | Chr12: 131193445-131196445 | NOC4L, Chr12: 131193445-131196445 |
| PIGZ | Chr3: 198178601-198181601 | PIGZ, Chr3: 198178601-198181601 |
| EFNA4 | Chr1: 153301336-153304336 | EFNA4, Chr1: 153301336-153304336 |
| ELA2 | Chr19: 801790-804790 | ELA2, Chr19: 801790-804790 |
| DKFZp761E198 | Chr11: 65302898-65305898 | DKFZp761E198, Chr11: 65302898-65305898 |
| AMH | Chr19: 2198612-2201612 | AMH, Chr19: 2198612-2201612 |
| LOC554235 | Chr19: 55708259-55711259 | LOC554235, Chr19: 55708259-55711259 |
| USP29 | Chr19: 62321820-62324820 | USP29, Chr19: 62321820-62324820 |
| C14orf79 | Chr14: 104522160-104525160 | C14orf79, Chr14: 104522160-104525160 |
| LOC646174 | Chr16: 3483922-3486922 | LOC646174, Chr16: 3483922-3486922 |
| PSG2 | Chr19: 48277154-48280154 | PSG2, Chr19: 48277154-48280154 |
| KLHL30 | Chr2: 238712655-238715655 | KLHL30, Chr2: 238712655-238715655 |
| SOX21 | Chr13: 94160890-94163890 | SOX21, Chr13: 94160890-94163890 |
| ADRA2C | Chr4: 3736593-3739593 | ADRA2C, Chr4: 3736593-3739593 |
| IRX2 | Chr5: 2803269-2806269 | IRX2, Chr5: 2803269-2806269 |
| THBD | Chr20: 22976801-22979801 | THBD, Chr20: 22976801-22979801 |
| KLF11 | Chr2: 10099632-10102632 | KLF11, Chr2: 10099632-10102632 |
| BBC3 | Chr19: 52424791-52427791 | BBC3, Chr19: 52424791-52427791 |
| RHPN2 | Chr19: 38246134-38249134 | RHPN2, Chr19: 38246134-38249134 |
| CENPB | Chr20: 3713837-3716837 | CENPB, Chr20: 3713837-3716837 |
| MTA2 | Chr11: 62124379-62127379 | MTA2, Chr11: 62124379-62127379 |
| HES6 | Chr2: 238811909-238814909 | HES6, Chr2: 238811909-238814909 |
| FASN | Chr17: 77647895-77650895 | FASN, Chr17: 77647895-77650895 |
| MBD1 | Chr18: 46060642-46063642 | MBD1, Chr18: 46060642-46063642 |
| TMEM80 | Chr11: 684115-687115 | TMEM80, Chr11: 684115-687115 |
| COASY | Chr17: 37966117-37969117 | COASY, Chr17: 37966117-37969117 |
| NFATC1 | Chr18: 75259813-75262813 | NFATC1, Chr18: 75259813-75262813 |
| RHOF | Chr12: 120714477-120717477 | RHOF, Chr12: 120714477-120717477 |
| KIFC2 | Chr8: 145661045-145664045 | KIFC2, Chr8: 145661045-145664045 |
| C2orf55 | Chr2: 98917616-98920616 | C2orf55, Chr2: 98917616-98920616 |
| MOBKL2A | Chr19: 2045769-2048769 | MOBKL2A, Chr19: 2045769-2048769 |
| WWC2 | Chr4: 184255956-184258956 | WWC2, Chr4: 184255956-184258956 |
| C19orf36 | Chr19: 2046367-2049367 | C19orf36, Chr19: 2046367-2049367 |
| ARRDC2 | Chr19: 17978476-17981476 | ARRDC2, Chr19: 17978476-17981476 |
| C16orf59 | Chr16: 2448615-2451615 | C16orf59, Chr16: 2448615-2451615 |
| MFSD10 | Chr4: 2904067-2907067 | MFSD10, Chr4: 2904067-2907067 |
| SCUBE3 | Chr6: 35288667-35291667 | SCUBE3, Chr6: 35288667-35291667 |
| PEG10 | Chr7: 94122072-94125072 | PEG10, Chr7: 94122072-94125072 |
| TRABD | Chr22: 48964986-48967986 | TRABD, Chr22: 48964986-48967986 |
| ZNF331 | Chr19: 58748872-58751872 | ZNF331, Chr19: 58748872-58751872 |
| DDX4 | Chr5: 55069032-55072032 | DDX4, Chr5: 55069032-55072032 |
| PKN1 | Chr19: 14410585-14413585 | PKN1, Chr19: 14410585-14413585 |
| RAX2 | Chr19: 3721719-3724719 | RAX2, Chr19: 3721719-3724719 |
| GSC2 | Chr22: 17516296-17519296 | GSC2, Chr22: 17516296-17519296 |
| ANKRD57 | Chr2: 109727699-109730699 | ANKRD57, Chr2: 109727699-109730699 |
| HNRPDL | Chr4: 83568902-83571902 | HNRPDL, Chr4: 83568902-83571902 |
| BOLL | Chr2: 198357683-198360683 | BOLL, Chr2: 198357683-198360683 |
| C17orf82 | Chr17: 56842393-56845393 | C17orf82, Chr17: 56842393-56845393 |
| TMEM200B | Chr1: 29321494-29324494 | TMEM200B, Chr1: 29321494-29324494 |
| PAQR9 | Chr3: 144163368-144166368 | PAQR9, Chr3: 144163368-144166368 |
| TMEM52 | Chr1: 1839067-1842067 | TMEM52, Chr1: 1839067-1842067 |
| GRTP1 | Chr13: 113064964-113067964 | GRTP1, Chr13: 113064964-113067964 |
| SLC35D3 | Chr6: 137283594-137286594 | SLC35D3, Chr6: 137283594-137286594 |
| CPNE7 | Chr16: 88168176-88171176 | CPNE7, Chr16: 88168176-88171176 |
| CRLF1 | Chr19: 18577160-18580160 | CRLF1, Chr19: 18577160-18580160 |
| MAP7D2 | Chr23: 20043437-20046437 | MAP7D2, Chr23: 20043437-20046437 |
| TEAD3 | Chr6: 35571339-35574339 | TEAD3, Chr6: 35571339-35574339 |
| SLC6A3 | Chr5: 1497038-1500038 | SLC6A3, Chr5: 1497038-1500038 |
| GPX4 | Chr19: 1054148-1057148 | GPX4, Chr19: 1054148-1057148 |
| ICMT | Chr1: 6217131-6220131 | ICMT, Chr1: 6217131-6220131 |
| C19orf24 | Chr19: 1225019-1228019 | C19orf24, Chr19: 1225019-1228019 |
| WNK2 | Chr9: 94985532-94988532 | WNK2, Chr9: 94985532-94988532 |
| MEST | Chr7: 129917673-129920673 | MEST, Chr7: 129917673-129920673 |
| NNAT | Chr20: 35581520-35584520 | NNAT, Chr20: 35581520-35584520 |
| ZFP41 | Chr8: 144398983-144401983 | ZFP41, Chr8: 144398983-144401983 |
| WIF1 | Chr12: 63799883-63802883 | WIF1, Chr12: 63799883-63802883 |
| CLDN6 | Chr16: 3006689-3009689 | CLDN6, Chr16: 3006689-3009689 |
| SCRIB | Chr8: 144968037-144971037 | SCRIB, Chr8: 144968037-144971037 |
| UPB1 | Chr22: 23219750-23222750 | UPB1, Chr22: 23219750-23222750 |
| TSSK3 | Chr1: 32598948-32601948 | TSSK3, Chr1: 32598948-32601948 |
| AZU1 | Chr19: 777330-780330 | AZU1, Chr19: 777330-780330 |
| RTN4 | Chr2: 55128330-55131330 | RTN4, Chr2: 55128330-55131330 |
| PLVAP | Chr19: 17347637-17350637 | PLVAP, Chr19: 17347637-17350637 |
| CGB7 | Chr19: 54249309-54252309 | CGB7, Chr19: 54249309-54252309 |
| NPR2 | Chr9: 35780905-35783905 | NPR2, Chr9: 35780905-35783905 |
| ELA1 | Chr12: 50025230-50028230 | ELA1, Chr12: 50025230-50028230 |
| MGAT4B | Chr5: 179165047-179168047 | MGAT4B, Chr5: 179165047-179168047 |
| MAFK | Chr7: 1535393-1538393 | MAFK, Chr7: 1535393-1538393 |
| C19orf6 | Chr19: 970641-973641 | C19orf6, Chr19: 970641-973641 |
| CACNA1B | Chr9: 139890561-139893561 | CACNA1B, Chr9: 139890561-139893561 |
| SLC12A2 | Chr5: 127445881-127448881 | SLC12A2, Chr5: 127445881-127448881 |
| PHLDA2 | Chr11: 2905726-2908726 | PHLDA2, Chr11: 2905726-2908726 |
| TMEM16J | Chr11: 430511-433511 | TMEM16J, Chr11: 430511-433511 |
| PRKCZ | Chr1: 1970268-1973268 | PRKCZ, Chr1: 1970268-1973268 |
| MAFG | Chr17: 77477379-77480379 | MAFG, Chr17: 77477379-77480379 |
| C9orf163 | Chr9: 138496267-138499267 | C9orf163, Chr9: 138496267-138499267 |
| WDR86 | Chr7: 150736557-150739557 | WDR86, Chr7: 150736557-150739557 |
| KRTAP4-1 | Chr17: 36593173-36596173 | KRTAP4-1, Chr17: 36593173-36596173 |
| POMT1 | Chr9: 133366609-133369609 | POMT1, Chr9: 133366609-133369609 |
| ATP1A3 | Chr19: 47188722-47191722 | ATP1A3, Chr19: 47188722-47191722 |
| C1orf86 | Chr1: 2114574-2117574 | C1orf86, Chr1: 2114574-2117574 |
| RNF32 | Chr7: 156124661-156127661 | RNF32, Chr7: 156124661-156127661 |
| RPL26 | Chr17: 8225790-8228790 | RPL26, Chr17: 8225790-8228790 |
| HTF9C | Chr22: 18483268-18486268 | HTF9C, Chr22: 18483268-18486268 |
| ATP9B | Chr18: 74928884-74931884 | ATP9B, Chr18: 74928884-74931884 |
| SLC39A7 | Chr6: 33275080-33278080 | SLC39A7, Chr6: 33275080-33278080 |
| PITPNM1 | Chr11: 67026916-67029916 | PITPNM1, Chr11: 67026916-67029916 |
| LYNX1 | Chr8: 143853939-143856939 | LYNX1, Chr8: 143853939-143856939 |
| PEX6 | Chr6: 43053459-43056459 | PEX6, Chr6: 43053459-43056459 |
| CKM | Chr19: 50516474-50519474 | CKM, Chr19: 50516474-50519474 |
| ARID1B | Chr6: 157139277-157142277 | ARID1B, Chr6: 157139277-157142277 |
| ANKRD13D | Chr11: 66811894-66814894 | ANKRD13D, Chr11: 66811894-66814894 |
| RSHL1 | Chr19: 51008917-51011917 | RSHL1, Chr19: 51008917-51011917 |
| GPS1 | Chr17: 77601551-77604551 | GPS1, Chr17: 77601551-77604551 |
| CNNM2 | Chr10: 104666603-104669603 | CNNM2, Chr10: 104666603-104669603 |
| UTF1 | Chr10: 134892267-134895267 | UTF1, Chr10: 134892267-134895267 |
| SUMO3 | Chr21: 45060972-45063972 | SUMO3, Chr21: 45060972-45063972 |
| NRARP | Chr9: 139315024-139318024 | NRARP, Chr9: 139315024-139318024 |
| NFIX | Chr19: 12966083-12969083 | NFIX, Chr19: 12966083-12969083 |
| HES2 | Chr1: 6401066-6404066 | HES2, Chr1: 6401066-6404066 |
| OVOL1 | Chr11: 65309604-65312604 | OVOL1, Chr11: 65309604-65312604 |
| FASTK | Chr7: 150407384-150410384 | FASTK, Chr7: 150407384-150410384 |
| TIGD5 | Chr8: 144749863-144752863 | TIGD5, Chr8: 144749863-144752863 |
| AMDHD2 | Chr16: 2508927-2511927 | AMDHD2, Chr16: 2508927-2511927 |
| CACNA1H | Chr16: 1141741-1144741 | CACNA1H, Chr16: 1141741-1144741 |
| GFPT2 | Chr5: 179711421-179714421 | GFPT2, Chr5: 179711421-179714421 |
| RGS10 | Chr10: 121290712-121293712 | RGS10, Chr10: 121290712-121293712 |
| PRIMA1 | Chr14: 93323019-93326019 | PRIMA1, Chr14: 93323019-93326019 |
| PPP1R3F | Chr23: 49011760-49014760 | PPP1R3F, Chr23: 49011760-49014760 |
| MFSD3 | Chr8: 145703859-145706859 | MFSD3, Chr8: 145703859-145706859 |
| C2orf68 | Chr2: 85691163-85694163 | C2orf68, Chr2: 85691163-85694163 |
| ADAM11 | Chr17: 40190593-40193593 | ADAM11, Chr17: 40190593-40193593 |
| ZNF768 | Chr16: 30443911-30446911 | ZNF768, Chr16: 30443911-30446911 |
| FBN2 | Chr5: 127900134-127903134 | FBN2, Chr5: 127900134-127903134 |
| MAP3K10 | Chr19: 45387990-45390990 | MAP3K10, Chr19: 45387990-45390990 |
| AXIN1 | Chr16: 340965-343965 | AXIN1, Chr16: 340965-343965 |
| ABTB1 | Chr3: 128872970-128875970 | ABTB1, Chr3: 128872970-128875970 |
| SYNJ2 | Chr6: 158321406-158324406 | SYNJ2, Chr6: 158321406-158324406 |
| FAM69B | Chr9: 138725350-138728350 | FAM69B, Chr9: 138725350-138728350 |
| DEF8 | Chr16: 88541183-88544183 | DEF8, Chr16: 88541183-88544183 |
| DEF8 | Chr16: 88541151-88544151 | DEF8, Chr16: 88541151-88544151 |
| FANK1 | Chr10: 127573597-127576597 | FANK1, Chr10: 127573597-127576597 |
| WNT3 | Chr17: 42249581-42252581 | WNT3, Chr17: 42249581-42252581 |
| TMEM171 | Chr5: 72450657-72453657 | TMEM171, Chr5: 72450657-72453657 |
| C16orf79 | Chr16: 2199512-2202512 | C16orf79, Chr16: 2199512-2202512 |
| DMRTC2 | Chr19: 47039425-47042425 | DMRTC2, Chr19: 47039425-47042425 |
| SVOPL | Chr7: 137998009-138001009 | SVOPL, Chr7: 137998009-138001009 |
| KRBA1 | Chr7: 149041580-149044580 | KRBA1, Chr7: 149041580-149044580 |
| LAT | Chr16: 28902387-28905387 | LAT, Chr16: 28902387-28905387 |
| RGS19 | Chr20: 62179789-62182789 | RGS19, Chr20: 62179789-62182789 |
| PLCH2 | Chr1: 2396113-2399113 | PLCH2, Chr1: 2396113-2399113 |
| A26C2 | Chr14: 18621864-18624864 | A26C2, Chr14: 18621864-18624864 |
| C3orf55 | Chr3: 158742352-158745352 | C3orf55, Chr3: 158742352-158745352 |
| DMRT3 | Chr9: 965463-968463 | DMRT3, Chr9: 965463-968463 |
| ADRB1 | Chr10: 115792295-115795295 | ADRB1, Chr10: 115792295-115795295 |
| ADCY9 | Chr16: 4104687-4107687 | ADCY9, Chr16: 4104687-4107687 |
| C13orf27 | Chr13: 102222643-102225643 | C13orf27, Chr13: 102222643-102225643 |
| FGF22 | Chr19: 589425-592425 | FGF22, Chr19: 589425-592425 |
| RASSF7 | Chr11: 549949-552949 | RASSF7, Chr11: 549949-552949 |
| B3GALT6 | Chr1: 1155991-1158991 | B3GALT6, Chr1: 1155991-1158991 |
| HTR5A | Chr7: 154491978-154494978 | HTR5A, Chr7: 154491978-154494978 |
| TSSK6 | Chr19: 19485969-19488969 | TSSK6, Chr19: 19485969-19488969 |
| TERT | Chr5: 1346662-1349662 | TERT, Chr5: 1346662-1349662 |
| ALDH1A3 | Chr15: 99236031-99239031 | ALDH1A3, Chr15: 99236031-99239031 |
| CNTFR | Chr9: 34578222-34581222 | CNTFR, Chr9: 34578222-34581222 |
| FLJ33790 | Chr11: 74817381-74820381 | FLJ33790, Chr11: 74817381-74820381 |
| GPR27 | Chr3: 71884390-71887390 | GPR27, Chr3: 71884390-71887390 |
| CPEB1 | Chr15: 81112283-81115283 | CPEB1, Chr15: 81112283-81115283 |
| DIP2C | Chr10: 724106-727106 | DIP2C, Chr10: 724106-727106 |
| HS6ST1 | Chr2: 128791141-128794141 | HS6ST1, Chr2: 128791141-128794141 |
| GNAT1 | Chr3: 50202546-50205546 | GNAT1, Chr3: 50202546-50205546 |
| GAS7 | Chr17: 10041093-10044093 | GAS7, Chr17: 10041093-10044093 |
| TRAF3 | Chr14: 102312068-102315068 | TRAF3, Chr14: 102312068-102315068 |
| ZMYND19 | Chr9: 139603258-139606258 | ZMYND19, Chr9: 139603258-139606258 |
| CYGB | Chr17: 72043877-72046877 | CYGB, Chr17: 72043877-72046877 |
| WTIP | Chr19: 39663219-39666219 | WTIP, Chr19: 39663219-39666219 |
| NT5C | Chr17: 70637972-70640972 | NT5C, Chr17: 70637972-70640972 |
| SHANK1 | Chr19: 55910507-55913507 | SHANK1, Chr19: 55910507-55913507 |
| MGAT3 | Chr22: 38181770-38184770 | MGAT3, Chr22: 38181770-38184770 |
| RANBP1 | Chr22: 18483523-18486523 | RANBP1, Chr22: 18483523-18486523 |
| KRTAP17-1 | Chr17: 36723973-36726973 | KRTAP17-1, Chr17: 36723973-36726973 |
| NRIP3 | Chr11: 8980624-8983624 | NRIP3, Chr11: 8980624-8983624 |
| KCNQ4 | Chr1: 41020770-41023770 | KCNQ4, Chr1: 41020770-41023770 |
| FAM113A | Chr20: 2767832-2770832 | FAM113A, Chr20: 2767832-2770832 |
| MOCOS | Chr18: 32019977-32022977 | MOCOS, Chr18: 32019977-32022977 |
| S1PR3 | Chr9: 90794681-90797681 | S1PR3, Chr9: 90794681-90797681 |
| RASGRF2 | Chr5: 80290813-80293813 | RASGRF2, Chr5: 80290813-80293813 |
| FARP2 | Chr2: 241942883-241945883 | FARP2, Chr2: 241942883-241945883 |
| SLC16A3 | Chr17: 77778677-77781677 | SLC16A3, Chr17: 77778677-77781677 |
| DNLZ | Chr9: 138376562-138379562 | DNLZ, Chr9: 138376562-138379562 |
| PNMT | Chr17: 35076532-35079532 | PNMT, Chr17: 35076532-35079532 |
| C16orf86 | Chr16: 66256717-66259717 | C16orf86, Chr16: 66256717-66259717 |
| DIABLO | Chr12: 121275052-121278052 | DIABLO, Chr12: 121275052-121278052 |
| ARSA | Chr22: 49411973-49414973 | ARSA, Chr22: 49411973-49414973 |
| GNPTG | Chr16: 1340432-1343432 | GNPTG, Chr16: 1340432-1343432 |
| ZDHHC8 | Chr22: 18497864-18500864 | ZDHHC8, Chr22: 18497864-18500864 |
| TMEM25 | Chr11: 117905633-117908633 | TMEM25, Chr11: 117905633-117908633 |
| LYPD4 | Chr19: 47038848-47041848 | LYPD4, Chr19: 47038848-47041848 |
| SEC14L5 | Chr16: 4946818-4949818 | SEC14L5, Chr16: 4946818-4949818 |
| A26C3 | Chr22: 14666437-14669437 | A26C3, Chr22: 14666437-14669437 |
| MRGPRE | Chr11: 3208692-3211692 | MRGPRE, Chr11: 3208692-3211692 |
| HSF2BP | Chr21: 43902302-43905302 | HSF2BP, Chr21: 43902302-43905302 |
| HEXB | Chr5: 74015224-74018224 | HEXB, Chr5: 74015224-74018224 |
| PDZD8 | Chr10: 119123427-119126427 | PDZD8, Chr10: 119123427-119126427 |
| KIF1A | Chr2: 241406797-241409797 | KIF1A, Chr2: 241406797-241409797 |
| LOC389813 | Chr9: 138857187-138860187 | LOC389813, Chr9: 138857187-138860187 |
| FGFR3 | Chr4: 1763336-1766336 | FGFR3, Chr4: 1763336-1766336 |
| DNMT3A | Chr2: 25416778-25419778 | DNMT3A, Chr2: 25416778-25419778 |
| PDZD4 | Chr23: 152747697-152750697 | PDZD4, Chr23: 152747697-152750697 |
| LOC283951 | Chr16: 1417846-1420846 | LOC283951, Chr16: 1417846-1420846 |
| BTBD6 | Chr14: 104784552-104787552 | BTBD6, Chr14: 104784552-104787552 |
| CHKA | Chr11: 67643934-67646934 | CHKA, Chr11: 67643934-67646934 |
| C13orf15 | Chr13: 40928041-40931041 | C13orf15, Chr13: 40928041-40931041 |
| DGKQ | Chr4: 955844-958844 | DGKQ, Chr4: 955844-958844 |
| BAHCC1 | Chr17: 76986634-76989634 | BAHCC1, Chr17: 76986634-76989634 |
| CSPG5 | Chr3: 47593863-47596863 | CSPG5, Chr3: 47593863-47596863 |
| TPTE | Chr21: 10011291-10014291 | TPTE, Chr21: 10011291-10014291 |
| LTBP2 | Chr14: 74147287-74150287 | LTBP2, Chr14: 74147287-74150287 |
| GJB2 | Chr13: 19663614-19666614 | GJB2, Chr13: 19663614-19666614 |
| BTBD14A | Chr9: 138125452-138128452 | BTBD14A, Chr9: 138125452-138128452 |
| DUS3L | Chr19: 5740690-5743690 | DUS3L, Chr19: 5740690-5743690 |
| CRIP1 | Chr14: 105022801-105025801 | CRIP1, Chr14: 105022801-105025801 |
| C1orf70 | Chr1: 1464103-1467103 | C1orf70, Chr1: 1464103-1467103 |
| PODNL1 | Chr19: 13908789-13911789 | PODNL1, Chr19: 13908789-13911789 |
| DOK1 | Chr2: 74633867-74636867 | DOK1, Chr2: 74633867-74636867 |
| VPS16 | Chr20: 2767872-2770872 | VPS16, Chr20: 2767872-2770872 |
| TDRD6 | Chr6: 46762324-46765324 | TDRD6, Chr6: 46762324-46765324 |
| CHAD | Chr17: 45899726-45902726 | CHAD, Chr17: 45899726-45902726 |
| SLC26A1 | Chr4: 975724-978724 | SLC26A1, Chr4: 975724-978724 |
| MMP17 | Chr12: 130877393-130880393 | MMP17, Chr12: 130877393-130880393 |
| FREQ | Chr9: 131973177-131976177 | FREQ, Chr9: 131973177-131976177 |
| RALA | Chr7: 39628186-39631186 | RALA, Chr7: 39628186-39631186 |
| SLC26A1 | Chr4: 975683-978683 | SLC26A1, Chr4: 975683-978683 |
| C19orf63 | Chr19: 55670047-55673047 | C19orf63, Chr19: 55670047-55673047 |
| WNT2B | Chr1: 112851888-112854888 | WNT2B, Chr1: 112851888-112854888 |
| PQLC2 | Chr1: 19509826-19512826 | PQLC2, Chr1: 19509826-19512826 |
| TMUB1 | Chr7: 150409521-150412521 | TMUB1, Chr7: 150409521-150412521 |
| RIBC2 | Chr22: 44186743-44189743 | RIBC2, Chr22: 44186743-44189743 |
| MGLL | Chr3: 129022891-129025891 | MGLL, Chr3: 129022891-129025891 |
| ADAM33 | Chr20: 3609238-3612238 | ADAM33, Chr20: 3609238-3612238 |
| ADSSL1 | Chr14: 104260078-104263078 | ADSSL1, Chr14: 104260078-104263078 |
| PTCRA | Chr6: 42990204-42993204 | PTCRA, Chr6: 42990204-42993204 |
| TDRD12 | Chr19: 37901018-37904018 | TDRD12, Chr19: 37901018-37904018 |
| OPRL1 | Chr20: 62180431-62183431 | OPRL1, Chr20: 62180431-62183431 |
| LAT | Chr16: 28902147-28905147 | LAT, Chr16: 28902147-28905147 |
| CATSPER2 | Chr15: 41726831-41729831 | CATSPER2, Chr15: 41726831-41729831 |
| WDR88 | Chr19: 38313337-38316337 | WDR88, Chr19: 38313337-38316337 |
| EFNA1 | Chr1: 153365472-153368472 | EFNA1, Chr1: 153365472-153368472 |
| SLC19A1 | Chr21: 45785279-45788279 | SLC19A1, Chr21: 45785279-45788279 |
| ARX | Chr23: 24942486-24945486 | ARX, Chr23: 24942486-24945486 |
| RIMS1 | Chr6: 72651870-72654870 | RIMS1, Chr6: 72651870-72654870 |
| EXOC3L2 | Chr19: 50427809-50430809 | EXOC3L2, Chr19: 50427809-50430809 |
| CBLN3 | Chr14: 23967071-23970071 | CBLN3, Chr14: 23967071-23970071 |
| MT1A | Chr16: 55228578-55231578 | MT1A, Chr16: 55228578-55231578 |
| CMTM2 | Chr16: 65169351-65172351 | CMTM2, Chr16: 65169351-65172351 |
| BIN2 | Chr12: 50002705-50005705 | BIN2, Chr12: 50002705-50005705 |
| SPRED3 | Chr19: 43571278-43574278 | SPRED3, Chr19: 43571278-43574278 |
| SMARCD3 | Chr7: 150603253-150606253 | SMARCD3, Chr7: 150603253-150606253 |
| PCSK6 | Chr15: 99846210-99849210 | PCSK6, Chr15: 99846210-99849210 |
| TLL2 | Chr10: 98262158-98265158 | TLL2, Chr10: 98262158-98265158 |
| C1orf35 | Chr1: 226356145-226359145 | C1orf35, Chr1: 226356145-226359145 |
| FGF19 | Chr11: 69226787-69229787 | FGF19, Chr11: 69226787-69229787 |
| BHLHB4 | Chr20: 61107287-61110287 | BHLHB4, Chr20: 61107287-61110287 |
| PLXND1 | Chr3: 130806772-130809772 | PLXND1, Chr3: 130806772-130809772 |
| DEAF1 | Chr11: 684240-687240 | DEAF1, Chr11: 684240-687240 |
| ARL4C | Chr2: 235068932-235071932 | ARL4C, Chr2: 235068932-235071932 |
| NUDT16L1 | Chr16: 4682216-4685216 | NUDT16L1, Chr16: 4682216-4685216 |
| KLHL34 | Chr23: 21584869-21587869 | KLHL34, Chr23: 21584869-21587869 |
| GP1BB | Chr22: 18089565-18092565 | GP1BB, Chr22: 18089565-18092565 |
| PC | Chr11: 66480923-66483923 | PC, Chr11: 66480923-66483923 |
| AP2A2 | Chr11: 914340-917340 | AP2A2, Chr11: 914340-917340 |
| HOXD12 | Chr2: 176671275-176674275 | HOXD12, Chr2: 176671275-176674275 |
| GDPD3 | Chr16: 30030879-30033879 | GDPD3, Chr16: 30030879-30033879 |
| ARL10 | Chr5: 175723607-175726607 | ARL10, Chr5: 175723607-175726607 |
| PDXK | Chr21: 43961905-43964905 | PDXK, Chr21: 43961905-43964905 |
| TTC7A | Chr2: 47020316-47023316 | TTC7A, Chr2: 47020316-47023316 |
| COL18A1 | Chr21: 45648024-45651024 | COL18A1, Chr21: 45648024-45651024 |
| CDR2 | Chr16: 22291939-22294939 | CDR2, Chr16: 22291939-22294939 |
| BARX1 | Chr9: 95755929-95758929 | BARX1, Chr9: 95755929-95758929 |
| SLC22A3 | Chr6: 160687914-160690914 | SLC22A3, Chr6: 160687914-160690914 |
| BMP8B | Chr1: 40025620-40028620 | BMP8B, Chr1: 40025620-40028620 |
| UBE2J1 | Chr6: 90117838-90120838 | UBE2J1, Chr6: 90117838-90120838 |
| PITX2 | Chr4: 111776457-111779457 | PITX2, Chr4: 111776457-111779457 |
| HSF4 | Chr16: 65753357-65756357 | HSF4, Chr16: 65753357-65756357 |
| ANPEP | Chr15: 88157576-88160576 | ANPEP, Chr15: 88157576-88160576 |
| AKR7A2 | Chr1: 19509727-19512727 | AKR7A2, Chr1: 19509727-19512727 |
| DGAT1 | Chr8: 145519875-145522875 | DGAT1, Chr8: 145519875-145522875 |
| ACE | Chr17: 58906665-58909665 | ACE, Chr17: 58906665-58909665 |
| HNRNPD | Chr4: 83512673-83515673 | HNRNPD, Chr4: 83512673-83515673 |
| LYNX1 | Chr8: 143854246-143857246 | LYNX1, Chr8: 143854246-143857246 |
| KCTD6 | Chr3: 58451362-58454362 | KCTD6, Chr3: 58451362-58454362 |
| GALNT9 | Chr12: 131414478-131417478 | GALNT9, Chr12: 131414478-131417478 |
| TRIM50 | Chr7: 72378521-72381521 | TRIM50, Chr7: 72378521-72381521 |
| FKBP6 | Chr7: 72378735-72381735 | FKBP6, Chr7: 72378735-72381735 |
| WDR40C | Chr23: 125126115-125129115 | WDR40C, Chr23: 125126115-125129115 |
| QRICH2 | Chr17: 71813856-71816856 | QRICH2, Chr17: 71813856-71816856 |
| PLEKHG4 | Chr16: 65869489-65872489 | PLEKHG4, Chr16: 65869489-65872489 |
| PLEKHG4 | Chr16: 65869492-65872492 | PLEKHG4, Chr16: 65869492-65872492 |
| TUB | Chr11: 8057984-8060984 | TUB, Chr11: 8057984-8060984 |
| MGMT | Chr10: 131153955-131156955 | MGMT, Chr10: 131153955-131156955 |
| SHANK2 | Chr11: 70184020-70187020 | SHANK2, Chr11: 70184020-70187020 |
| AURKC | Chr19: 62432739-62435739 | AURKC, Chr19: 62432739-62435739 |
| LGALS3BP | Chr17: 74486156-74489156 | LGALS3BP, Chr17: 74486156-74489156 |
| LGR5 | Chr12: 70118579-70121579 | LGR5, Chr12: 70118579-70121579 |
| C19orf34 | Chr19: 1904048-1907048 | C19orf34, Chr19: 1904048-1907048 |
| IMP5 | Chr17: 41276552-41279552 | IMP5, Chr17: 41276552-41279552 |
| FZD9 | Chr7: 72484544-72487544 | FZD9, Chr7: 72484544-72487544 |
| PTPRU | Chr1: 29434133-29437133 | PTPRU, Chr1: 29434133-29437133 |
| SOCS3 | Chr17: 73866253-73869253 | SOCS3, Chr17: 73866253-73869253 |
| C19orf22 | Chr19: 862700-865700 | C19orf22, Chr19: 862700-865700 |
| PIWIL1 | Chr12: 129387066-129390066 | PIWIL1, Chr12: 129387066-129390066 |
| BNC1 | Chr15: 81742972-81745972 | BNC1, Chr15: 81742972-81745972 |
| MIB2 | Chr1: 1539246-1542246 | MIB2, Chr1: 1539246-1542246 |
| LHX3 | Chr9: 138233325-138236325 | LHX3, Chr9: 138233325-138236325 |
| SLC25A1 | Chr22: 17544785-17547785 | SLC25A1, Chr22: 17544785-17547785 |
| GPR25 | Chr1: 199107288-199110288 | GPR25, Chr1: 199107288-199110288 |
| UAP1L1 | Chr9: 139090273-139093273 | UAP1L1, Chr9: 139090273-139093273 |
| FAM132A | Chr1: 1170465-1173465 | FAM132A, Chr1: 1170465-1173465 |
| EGLN2 | Chr19: 45995387-45998387 | EGLN2, Chr19: 45995387-45998387 |
| TRAPPC5 | Chr19: 7650263-7653263 | TRAPPC5, Chr19: 7650263-7653263 |
| DDX51 | Chr12: 131193333-131196333 | DDX51, Chr12: 131193333-131196333 |
| CLDN11 | Chr3: 171617858-171620858 | CLDN11, Chr3: 171617858-171620858 |
| CLDN3 | Chr7: 72821036-72824036 | CLDN3, Chr7: 72821036-72824036 |
| GUCY2D | Chr17: 7845212-7848212 | GUCY2D, Chr17: 7845212-7848212 |
| NTSR1 | Chr20: 60809133-60812133 | NTSR1, Chr20: 60809133-60812133 |
| RBM44 | Chr2: 238370626-238373626 | RBM44, Chr2: 238370626-238373626 |
| KCNG2 | Chr18: 75723155-75726155 | KCNG2, Chr18: 75723155-75726155 |
| LOC201164 | Chr17: 17048857-17051857 | LOC201164, Chr17: 17048857-17051857 |
| TOX2 | Chr20: 41975405-41978405 | TOX2, Chr20: 41975405-41978405 |
| SCGB3A1 | Chr5: 179949593-179952593 | SCGB3A1, Chr5: 179949593-179952593 |
| ABBA-1 | Chr16: 69275955-69278955 | ABBA-1, Chr16: 69275955-69278955 |
| TUBGCP3 | Chr13: 112288982-112291982 | TUBGCP3, Chr13: 112288982-112291982 |
| AQP5 | Chr12: 48640045-48643045 | AQP5, Chr12: 48640045-48643045 |
| RXRB | Chr6: 33274910-33277910 | RXRB, Chr6: 33274910-33277910 |
| CLDN9 | Chr16: 3000706-3003706 | CLDN9, Chr16: 3000706-3003706 |
| C16orf42 | Chr16: 1340374-1343374 | C16orf42, Chr16: 1340374-1343374 |
| FEM1A | Chr19: 4741227-4744227 | FEM1A, Chr19: 4741227-4744227 |
| FLJ30851 | Chr2: 24249975-24252975 | FLJ30851, Chr2: 24249975-24252975 |
| COL25A1 | Chr4: 110441748-110444748 | COL25A1, Chr4: 110441748-110444748 |
| HOXA7 | Chr7: 27161321-27164321 | HOXA7, Chr7: 27161321-27164321 |
| THSD3 | Chr14: 77033412-77036412 | THSD3, Chr14: 77033412-77036412 |
| PKMYT1 | Chr16: 2968975-2971975 | PKMYT1, Chr16: 2968975-2971975 |
| JOSD2 | Chr19: 55704657-55707657 | JOSD2, Chr19: 55704657-55707657 |
| KIAA0408 | Chr6: 127837052-127840052 | KIAA0408, Chr6: 127837052-127840052 |
| SMC1B | Chr22: 44186664-44189664 | SMC1B, Chr22: 44186664-44189664 |
| RNF207 | Chr1: 6187275-6190275 | RNF207, Chr1: 6187275-6190275 |
| RSU1 | Chr10: 16897959-16900959 | RSU1, Chr10: 16897959-16900959 |
| SPDYA | Chr2: 28885720-28888720 | SPDYA, Chr2: 28885720-28888720 |
| ZNF331 | Chr19: 58731644-58734644 | ZNF331, Chr19: 58731644-58734644 |
| ZNF354C | Chr5: 178418712-178421712 | ZNF354C, Chr5: 178418712-178421712 |
| PTGR1 | Chr9: 113400032-113403032 | PTGR1, Chr9: 113400032-113403032 |
| ZNF511 | Chr10: 134970912-134973912 | ZNF511, Chr10: 134970912-134973912 |
| MPG | Chr16: 66668-69668 | MPG, Chr16: 66668-69668 |
| TDRD9 | Chr14: 103463069-103466069 | TDRD9, Chr14: 103463069-103466069 |
| KRTAP5-2 | Chr11: 1574600-1577600 | KRTAP5-2, Chr11: 1574600-1577600 |
| AATK | Chr17: 76752967-76755967 | AATK, Chr17: 76752967-76755967 |
| FADS6 | Chr17: 70399800-70402800 | FADS6, Chr17: 70399800-70402800 |
| ARHGEF10L | Chr1: 17737416-17740416 | ARHGEF10L, Chr1: 17737416-17740416 |
| ZPBP2 | Chr17: 35276480-35279480 | ZPBP2, Chr17: 35276480-35279480 |
| TUBA8 | Chr22: 16972058-16975058 | TUBA8, Chr22: 16972058-16975058 |
| DAPK2 | Chr15: 62124074-62127074 | DAPK2, Chr15: 62124074-62127074 |
| ANKDD1A | Chr15: 62989653-62992653 | ANKDD1A, Chr15: 62989653-62992653 |
| HPCA | Chr1: 33123184-33126184 | HPCA, Chr1: 33123184-33126184 |
| MPDU1 | Chr17: 7426353-7429353 | MPDU1, Chr17: 7426353-7429353 |
| CNIH2 | Chr11: 65800771-65803771 | CNIH2, Chr11: 65800771-65803771 |
| KIAA0323 | Chr14: 23967480-23970480 | KIAA0323, Chr14: 23967480-23970480 |
| NAT14 | Chr19: 60686905-60689905 | NAT14, Chr19: 60686905-60689905 |
| TRAM2 | Chr6: 52548321-52551321 | TRAM2, Chr6: 52548321-52551321 |
| ZNF717 | Chr3: 75915445-75918445 | ZNF717, Chr3: 75915445-75918445 |
| FIGNL1 | Chr7: 50484082-50487082 | FIGNL1, Chr7: 50484082-50487082 |
| AURKC | Chr19: 62432688-62435688 | AURKC, Chr19: 62432688-62435688 |
| LOC51252 | Chr2: 97014528-97017528 | LOC51252, Chr2: 97014528-97017528 |
| INSC | Chr11: 15091559-15094559 | INSC, Chr11: 15091559-15094559 |
| ANKRD30A | Chr10: 37453290-37456290 | ANKRD30A, Chr10: 37453290-37456290 |
| ADSSL1 | Chr14: 104265772-104268772 | ADSSL1, Chr14: 104265772-104268772 |
| POU4F3 | Chr5: 145697368-145700368 | POU4F3, Chr5: 145697368-145700368 |
| PDE4A | Chr19: 10423132-10426132 | PDE4A, Chr19: 10423132-10426132 |
| DRD5 | Chr4: 9390855-9393855 | DRD5, Chr4: 9390855-9393855 |
| RXFP3 | Chr5: 33970747-33973747 | RXFP3, Chr5: 33970747-33973747 |
| PRR18 | Chr6: 166640361-166643361 | PRR18, Chr6: 166640361-166643361 |
| DNASE1L2 | Chr16: 2224968-2227968 | DNASE1L2, Chr16: 2224968-2227968 |
| GATA2 | Chr3: 129693218-129696218 | GATA2, Chr3: 129693218-129696218 |
| UNKL | Chr16: 1368182-1371182 | UNKL, Chr16: 1368182-1371182 |
| TRAPPC5 | Chr19: 7650206-7653206 | TRAPPC5, Chr19: 7650206-7653206 |
| TRAPPC5 | Chr19: 7650260-7653260 | TRAPPC5, Chr19: 7650260-7653260 |
| SGCE | Chr7: 94121957-94124957 | SGCE, Chr7: 94121957-94124957 |
| CRMP1 | Chr4: 5944186-5947186 | CRMP1, Chr4: 5944186-5947186 |
| TFAP2E | Chr1: 35810057-35813057 | TFAP2E, Chr1: 35810057-35813057 |
| ASPSCR1 | Chr17: 77527214-77530214 | ASPSCR1, Chr17: 77527214-77530214 |
| GINS2 | Chr16: 84278581-84281581 | GINS2, Chr16: 84278581-84281581 |
| SSTR4 | Chr20: 22962556-22965556 | SSTR4, Chr20: 22962556-22965556 |
| CDX1 | Chr5: 149525036-149528036 | CDX1, Chr5: 149525036-149528036 |
| CCK | Chr3: 42279899-42282899 | CCK, Chr3: 42279899-42282899 |
| ADCK5 | Chr8: 145567038-145570038 | ADCK5, Chr8: 145567038-145570038 |
| GLDC | Chr9: 6634192-6637192 | GLDC, Chr9: 6634192-6637192 |
| GCLM | Chr1: 94146100-94149100 | GCLM, Chr1: 94146100-94149100 |
| PODXL2 | Chr3: 128829228-128832228 | PODXL2, Chr3: 128829228-128832228 |
| ARRB1 | Chr11: 74739021-74742021 | ARRB1, Chr11: 74739021-74742021 |
| MOV10L1 | Chr22: 48869120-48872120 | MOV10L1, Chr22: 48869120-48872120 |
| SDC3 | Chr1: 31152567-31155567 | SDC3, Chr1: 31152567-31155567 |
| LYSMD4 | Chr15: 98089649-98092649 | LYSMD4, Chr15: 98089649-98092649 |
| NKAIN4 | Chr20: 61354835-61357835 | NKAIN4, Chr20: 61354835-61357835 |
| GAL | Chr11: 68207058-68210058 | GAL, Chr11: 68207058-68210058 |
| PTRH1 | Chr9: 129516257-129519257 | PTRH1, Chr9: 129516257-129519257 |
| ATP2A3 | Chr17: 3812985-3815985 | ATP2A3, Chr17: 3812985-3815985 |
| C16orf48 | Chr16: 66256629-66259629 | C16orf48, Chr16: 66256629-66259629 |
| RASGRP1 | Chr15: 36642799-36645799 | RASGRP1, Chr15: 36642799-36645799 |
| RCCD1 | Chr15: 89297609-89300609 | RCCD1, Chr15: 89297609-89300609 |
| HDAC10 | Chr22: 49030441-49033441 | HDAC10, Chr22: 49030441-49033441 |
| DPF1 | Chr19: 43405128-43408128 | DPF1, Chr19: 43405128-43408128 |
| PPP1R14A | Chr19: 43437512-43440512 | PPP1R14A, Chr19: 43437512-43440512 |
| PRR7 | Chr5: 176804901-176807901 | PRR7, Chr5: 176804901-176807901 |
| TSPO | Chr22: 41875978-41878978 | TSPO, Chr22: 41875978-41878978 |
| AKR1CL2 | Chr10: 4856901-4859901 | AKR1CL2, Chr10: 4856901-4859901 |
| RGS19 | Chr20: 62180268-62183268 | RGS19, Chr20: 62180268-62183268 |
| ENTPD2 | Chr9: 139066826-139069826 | ENTPD2, Chr9: 139066826-139069826 |
| CMTM1 | Chr16: 65156350-65159350 | CMTM1, Chr16: 65156350-65159350 |
| FTMT | Chr5: 121214048-121217048 | FTMT, Chr5: 121214048-121217048 |
| RPESP | Chr8: 74166561-74169561 | RPESP, Chr8: 74166561-74169561 |
| SLC1A7 | Chr1: 53379377-53382377 | SLC1A7, Chr1: 53379377-53382377 |
| NOX5 | Chr15: 67008417-67011417 | NOX5, Chr15: 67008417-67011417 |
| SPESP1 | Chr15: 67008418-67011418 | SPESP1, Chr15: 67008418-67011418 |
| MLX | Chr17: 37971103-37974103 | MLX, Chr17: 37971103-37974103 |
| ZBTB40 | Chr1: 22649430-22652430 | ZBTB40, Chr1: 22649430-22652430 |
| FAM131C | Chr1: 16271214-16274214 | FAM131C, Chr1: 16271214-16274214 |
| NTN2L | Chr16: 2460000-2463000 | NTN2L, Chr16: 2460000-2463000 |
| NPEPL1 | Chr20: 56699767-56702767 | NPEPL1, Chr20: 56699767-56702767 |
| FTHL17 | Chr23: 30998591-31001591 | FTHL17, Chr23: 30998591-31001591 |
| DAGLA | Chr11: 61202985-61205985 | DAGLA, Chr11: 61202985-61205985 |
| TACC2 | Chr10: 123911594-123914594 | TACC2, Chr10: 123911594-123914594 |
| 5-Sep | Chr22: 18080486-18083486 | 40791, Chr22: 18080486-18083486 |
| PLEC1 | Chr8: 145118128-145121128 | PLEC1, Chr8: 145118128-145121128 |
| CABLES2 | Chr20: 60414234-60417234 | CABLES2, Chr20: 60414234-60417234 |
| AFAR3 | Chr1: 19471522-19474522 | AFAR3, Chr1: 19471522-19474522 |
| YY2 | Chr23: 21783023-21786023 | YY2, Chr23: 21783023-21786023 |
| LOC374569 | Chr14: 103620300-103623300 | LOC374569, Chr14: 103620300-103623300 |
| KIF7 | Chr15: 87991078-87994078 | KIF7, Chr15: 87991078-87994078 |
| PNLDC1 | Chr6: 160139790-160142790 | PNLDC1, Chr6: 160139790-160142790 |
| EXOC7 | Chr17: 71609951-71612951 | EXOC7, Chr17: 71609951-71612951 |
| C9orf106 | Chr9: 131121615-131124615 | C9orf106, Chr9: 131121615-131124615 |
| STK31 | Chr7: 23715021-23718021 | STK31, Chr7: 23715021-23718021 |
| KIF19 | Chr17: 69832445-69835445 | KIF19, Chr17: 69832445-69835445 |
| PCDH21 | Chr10: 85942996-85945996 | PCDH21, Chr10: 85942996-85945996 |
| MAGEL2 | Chr15: 21440768-21443768 | MAGEL2, Chr15: 21440768-21443768 |
| AMZ1 | Chr7: 2684188-2687188 | AMZ1, Chr7: 2684188-2687188 |
| ADAMTSL2 | Chr9: 135388295-135391295 | ADAMTSL2, Chr9: 135388295-135391295 |
| LDOC1L | Chr22: 43271169-43274169 | LDOC1L, Chr22: 43271169-43274169 |
| SNX18 | Chr5: 53847849-53850849 | SNX18, Chr5: 53847849-53850849 |
| FZD2 | Chr17: 39988950-39991950 | FZD2, Chr17: 39988950-39991950 |
| TPSB2 | Chr16: 1218686-1221686 | TPSB2, Chr16: 1218686-1221686 |
| FUT4 | Chr11: 93915274-93918274 | FUT4, Chr11: 93915274-93918274 |
| CDK5R2 | Chr2: 219531141-219534141 | CDK5R2, Chr2: 219531141-219534141 |
| LRRC26 | Chr9: 139182812-139185812 | LRRC26, Chr9: 139182812-139185812 |
| LOC90826 | Chr4: 148823230-148826230 | LOC90826, Chr4: 148823230-148826230 |
| GAS2L1 | Chr22: 28031496-28034496 | GAS2L1, Chr22: 28031496-28034496 |
| ARC | Chr8: 143691335-143694335 | ARC, Chr8: 143691335-143694335 |
| DHODH | Chr16: 70598643-70601643 | DHODH, Chr16: 70598643-70601643 |
| MEST | Chr7: 129916908-129919908 | MEST, Chr7: 129916908-129919908 |
| HS1BP3 | Chr2: 20712845-20715845 | HS1BP3, Chr2: 20712845-20715845 |
| CHRD | Chr3: 185579054-185582054 | CHRD, Chr3: 185579054-185582054 |
| GAS6 | Chr13: 113545412-113548412 | GAS6, Chr13: 113545412-113548412 |
| FOXRED2 | Chr22: 35231536-35234536 | FOXRED2, Chr22: 35231536-35234536 |
| NOTCH3 | Chr19: 15171292-15174292 | NOTCH3, Chr19: 15171292-15174292 |
| ZNHIT2 | Chr11: 64640246-64643246 | ZNHIT2, Chr11: 64640246-64643246 |
| SPIB | Chr19: 55612527-55615527 | SPIB, Chr19: 55612527-55615527 |
| GPR123 | Chr10: 134749898-134752898 | GPR123, Chr10: 134749898-134752898 |
| C9orf47 | Chr9: 90794162-90797162 | C9orf47, Chr9: 90794162-90797162 |
| C9orf129 | Chr9: 95147017-95150017 | C9orf129, Chr9: 95147017-95150017 |
| VWCE | Chr11: 60817864-60820864 | VWCE, Chr11: 60817864-60820864 |
| ZNF135 | Chr19: 63260923-63263923 | ZNF135, Chr19: 63260923-63263923 |
| SBF1 | Chr22: 49258820-49261820 | SBF1, Chr22: 49258820-49261820 |
| LMF1 | Chr16: 959485-962485 | LMF1, Chr16: 959485-962485 |
| WSB1 | Chr17: 22643732-22646732 | WSB1, Chr17: 22643732-22646732 |
| SRD5A2 | Chr2: 31658044-31661044 | SRD5A2, Chr2: 31658044-31661044 |
| GGN | Chr19: 43569008-43572008 | GGN, Chr19: 43569008-43572008 |
| IMP4 | Chr2: 130815458-130818458 | IMP4, Chr2: 130815458-130818458 |
| C10orf125 | Chr10: 135020019-135023019 | C10orf125, Chr10: 135020019-135023019 |
| GJB6 | Chr13: 19702956-19705956 | GJB6, Chr13: 19702956-19705956 |
| CAMKK1 | Chr17: 3741586-3744586 | CAMKK1, Chr17: 3741586-3744586 |
| PPP1R16B | Chr20: 36866261-36869261 | PPP1R16B, Chr20: 36866261-36869261 |
| FBXW5 | Chr9: 138957494-138960494 | FBXW5, Chr9: 138957494-138960494 |
| SLC9A3 | Chr5: 576049-579049 | SLC9A3, Chr5: 576049-579049 |
| TBRG4 | Chr7: 45116342-45119342 | TBRG4, Chr7: 45116342-45119342 |
| RBM46 | Chr4: 155920449-155923449 | RBM46, Chr4: 155920449-155923449 |
| MGLL | Chr3: 129023241-129026241 | MGLL, Chr3: 129023241-129026241 |
| SOD1 | Chr21: 31952305-31955305 | SOD1, Chr21: 31952305-31955305 |
| PLAGL1 | Chr6: 144369734-144372734 | PLAGL1, Chr6: 144369734-144372734 |
| PRAF2 | Chr23: 48817106-48820106 | PRAF2, Chr23: 48817106-48820106 |
| VSTM2L | Chr20: 35963412-35966412 | VSTM2L, Chr20: 35963412-35966412 |
| TUBGCP2 | Chr10: 134971134-134974134 | TUBGCP2, Chr10: 134971134-134974134 |
| GBL | Chr16: 2193950-2196950 | GBL, Chr16: 2193950-2196950 |
| SH3BP2 | Chr4: 2763047-2766047 | SH3BP2, Chr4: 2763047-2766047 |
| TNFRSF1B | Chr1: 12148146-12151146 | TNFRSF1B, Chr1: 12148146-12151146 |
| MUPCDH | Chr11: 613507-616507 | MUPCDH, Chr11: 613507-616507 |
| AKR7A3 | Chr1: 19486367-19489367 | AKR7A3, Chr1: 19486367-19489367 |
| CMTM1 | Chr16: 65156294-65159294 | CMTM1, Chr16: 65156294-65159294 |
| KY | Chr3: 135851054-135854054 | KY, Chr3: 135851054-135854054 |
| PRKCDBP | Chr11: 6296816-6299816 | PRKCDBP, Chr11: 6296816-6299816 |
| AEBP1 | Chr7: 44108984-44111984 | AEBP1, Chr7: 44108984-44111984 |
| HHIPL1 | Chr14: 99179732-99182732 | HHIPL1, Chr14: 99179732-99182732 |
| SLC25A31 | Chr4: 128869523-128872523 | SLC25A31, Chr4: 128869523-128872523 |
| SPACA1 | Chr6: 88812725-88815725 | SPACA1, Chr6: 88812725-88815725 |
| CABIN1 | Chr22: 22736264-22739264 | CABIN1, Chr22: 22736264-22739264 |
| AIFM3 | Chr22: 19647933-19650933 | AIFM3, Chr22: 19647933-19650933 |
| AIFM3 | Chr22: 19647950-19650950 | AIFM3, Chr22: 19647950-19650950 |
| AMIGO1 | Chr1: 109852359-109855359 | AMIGO1, Chr1: 109852359-109855359 |
| DOK7 | Chr4: 3433330-3436330 | DOK7, Chr4: 3433330-3436330 |
| MAPRE2 | Chr18: 30873888-30876888 | MAPRE2, Chr18: 30873888-30876888 |
| STK31 | Chr7: 23714862-23717862 | STK31, Chr7: 23714862-23717862 |
| PIWIL2 | Chr8: 22187300-22190300 | PIWIL2, Chr8: 22187300-22190300 |
| MTNR1B | Chr11: 92340936-92343936 | MTNR1B, Chr11: 92340936-92343936 |
| ADAD2 | Chr16: 82780744-82783744 | ADAD2, Chr16: 82780744-82783744 |
| FLJ32658 | Chr19: 54581817-54584817 | FLJ32658, Chr19: 54581817-54584817 |
| OCA2 | Chr15: 26016553-26019553 | OCA2, Chr15: 26016553-26019553 |
| TNFRSF25 | Chr1: 6447342-6450342 | TNFRSF25, Chr1: 6447342-6450342 |
| FBLN2 | Chr3: 13564124-13567124 | FBLN2, Chr3: 13564124-13567124 |
| PABPC3 | Chr13: 24566775-24569775 | PABPC3, Chr13: 24566775-24569775 |
| CD24 | Chr24: 19612593-19615593 | CD24, Chr24: 19612593-19615593 |
| FAM27L | Chr17: 21747996-21750996 | FAM27L, Chr17: 21747996-21750996 |
| MAEL | Chr1: 165223642-165226642 | MAEL, Chr1: 165223642-165226642 |
| RGS3 | Chr9: 115264329-115267329 | RGS3, Chr9: 115264329-115267329 |
| PLEC1 | Chr8: 145088553-145091553 | PLEC1, Chr8: 145088553-145091553 |
| HSF5 | Chr17: 53919258-53922258 | HSF5, Chr17: 53919258-53922258 |
| TCEB3CL | Chr18: 42808711-42811711 | TCEB3CL, Chr18: 42808711-42811711 |
| YDJC | Chr22: 20312840-20315840 | YDJC, Chr22: 20312840-20315840 |
| TMEM16H | Chr19: 17305138-17308138 | TMEM16H, Chr19: 17305138-17308138 |
| SPRN | Chr10: 135086611-135089611 | SPRN, Chr10: 135086611-135089611 |
| FAM50B | Chr6: 3793130-3796130 | FAM50B, Chr6: 3793130-3796130 |
| NKPD1 | Chr19: 50347450-50350450 | NKPD1, Chr19: 50347450-50350450 |
| C19orf26 | Chr19: 1187490-1190490 | C19orf26, Chr19: 1187490-1190490 |
| ISYNA1 | Chr19: 18408443-18411443 | ISYNA1, Chr19: 18408443-18411443 |
| C11orf9 | Chr11: 61277936-61280936 | C11orf9, Chr11: 61277936-61280936 |
| SIX6 | Chr14: 60044190-60047190 | SIX6, Chr14: 60044190-60047190 |
| EMILIN1 | Chr2: 27153509-27156509 | EMILIN1, Chr2: 27153509-27156509 |
| DND1 | Chr5: 140031855-140034855 | DND1, Chr5: 140031855-140034855 |
| RBMXL2 | Chr11: 7065240-7068240 | RBMXL2, Chr11: 7065240-7068240 |
| NPY | Chr7: 24288833-24291833 | NPY, Chr7: 24288833-24291833 |
| FOXRED2 | Chr22: 35231594-35234594 | FOXRED2, Chr22: 35231594-35234594 |
| LOC162073 | Chr16: 19031282-19034282 | LOC162073, Chr16: 19031282-19034282 |
| POLR2E | Chr19: 1044891-1047891 | POLR2E, Chr19: 1044891-1047891 |
| SLC16A3 | Chr17: 77778081-77781081 | SLC16A3, Chr17: 77778081-77781081 |
| EML3 | Chr11: 62135313-62138313 | EML3, Chr11: 62135313-62138313 |
| OTUD6A | Chr23: 69197565-69200565 | OTUD6A, Chr23: 69197565-69200565 |
| REC8 | Chr14: 23709573-23712573 | REC8, Chr14: 23709573-23712573 |
| GPR78 | Chr4: 8631690-8634690 | GPR78, Chr4: 8631690-8634690 |
| KIAA0495 | Chr1: 3652246-3655246 | KIAA0495, Chr1: 3652246-3655246 |
| NELF | Chr9: 139472075-139475075 | NELF, Chr9: 139472075-139475075 |
| HLA-A29.1 | Chr6: 30016804-30019804 | HLA-A29.1, Chr6: 30016804-30019804 |
| HLA-A | Chr6: 30016809-30019809 | HLA-A, Chr6: 30016809-30019809 |
| HDGFL1 | Chr6: 22676156-22679156 | HDGFL1, Chr6: 22676156-22679156 |
| MFI2 | Chr3: 198239583-198242583 | MFI2, Chr3: 198239583-198242583 |
| AGPAT2 | Chr9: 138700232-138703232 | AGPAT2, Chr9: 138700232-138703232 |
| HUS1B | Chr6: 600464-603464 | HUS1B, Chr6: 600464-603464 |
| TUBA3D | Chr2: 131948635-131951635 | TUBA3D, Chr2: 131948635-131951635 |
| BOLA1 | Chr1: 148136324-148139324 | BOLA1, Chr1: 148136324-148139324 |
| HPDL | Chr1: 45563631-45566631 | HPDL, Chr1: 45563631-45566631 |
| PLEKHG4 | Chr16: 65869050-65872050 | PLEKHG4, Chr16: 65869050-65872050 |
| OPRD1 | Chr1: 29009740-29012740 | OPRD1, Chr1: 29009740-29012740 |
| NAPRT1 | Chr8: 144730136-144733136 | NAPRT1, Chr8: 144730136-144733136 |
| PRIC285 | Chr20: 61674536-61677536 | PRIC285, Chr20: 61674536-61677536 |
| MRPL28 | Chr16: 359041-362041 | MRPL28, Chr16: 359041-362041 |
| LASS3 | Chr15: 98900948-98903948 | LASS3, Chr15: 98900948-98903948 |
| DLEU7 | Chr13: 50314386-50317386 | DLEU7, Chr13: 50314386-50317386 |
| DACT2 | Chr6: 168461751-168464751 | DACT2, Chr6: 168461751-168464751 |
| PDPN | Chr1: 13781338-13784338 | PDPN, Chr1: 13781338-13784338 |
| LHX3 | Chr9: 138235276-138238276 | LHX3, Chr9: 138235276-138238276 |
| KRT7 | Chr12: 50911720-50914720 | KRT7, Chr12: 50911720-50914720 |
| ABCC8 | Chr11: 17453525-17456525 | ABCC8, Chr11: 17453525-17456525 |
| CHRNA4 | Chr20: 61461639-61464639 | CHRNA4, Chr20: 61461639-61464639 |
| GAPDHS | Chr19: 40714653-40717653 | GAPDHS, Chr19: 40714653-40717653 |
| TCP11 | Chr6: 35215665-35218665 | TCP11, Chr6: 35215665-35218665 |
| WBP2NL | Chr22: 40723237-40726237 | WBP2NL, Chr22: 40723237-40726237 |
| CR1L | Chr1: 205883580-205886580 | CR1L, Chr1: 205883580-205886580 |
| GAB4 | Chr22: 15867612-15870612 | GAB4, Chr22: 15867612-15870612 |
| VWA1 | Chr1: 1359271-1362271 | VWA1, Chr1: 1359271-1362271 |
| INSL6 | Chr9: 5174118-5177118 | INSL6, Chr9: 5174118-5177118 |
| RNF113B | Chr13: 97626022-97629022 | RNF113B, Chr13: 97626022-97629022 |
| LAMC3 | Chr9: 132872824-132875824 | LAMC3, Chr9: 132872824-132875824 |
| SLC7A4 | Chr22: 19715347-19718347 | SLC7A4, Chr22: 19715347-19718347 |
| ECE2 | Chr3: 185474992-185477992 | ECE2, Chr3: 185474992-185477992 |
| MMP11 | Chr22: 22443535-22446535 | MMP11, Chr22: 22443535-22446535 |
| NDN | Chr15: 21482043-21485043 | NDN, Chr15: 21482043-21485043 |
| ATP2B2 | Chr3: 10520768-10523768 | ATP2B2, Chr3: 10520768-10523768 |
| TDRD1 | Chr10: 115927518-115930518 | TDRD1, Chr10: 115927518-115930518 |
| LDHAL6A | Chr11: 18432506-18435506 | LDHAL6A, Chr11: 18432506-18435506 |
| C10orf54 | Chr10: 73201843-73204843 | C10orf54, Chr10: 73201843-73204843 |
| LSP1 | Chr11: 1847229-1850229 | LSP1, Chr11: 1847229-1850229 |
| TP73 | Chr1: 3595595-3598595 | TP73, Chr1: 3595595-3598595 |
| C1orf65 | Chr1: 221631890-221634890 | C1orf65, Chr1: 221631890-221634890 |
| TMPRSS12 | Chr12: 49521467-49524467 | TMPRSS12, Chr12: 49521467-49524467 |
| FRMD1 | Chr6: 168217881-168220881 | FRMD1, Chr6: 168217881-168220881 |
| SPATA22 | Chr17: 3320287-3323287 | SPATA22, Chr17: 3320287-3323287 |
| PLEC1 | Chr8: 145098576-145101576 | PLEC1, Chr8: 145098576-145101576 |
| ALX3 | Chr1: 110413345-110416345 | ALX3, Chr1: 110413345-110416345 |
| FAM123A | Chr13: 24642357-24645357 | FAM123A, Chr13: 24642357-24645357 |
| SOX18 | Chr20: 62149923-62152923 | SOX18, Chr20: 62149923-62152923 |
| KCNA5 | Chr12: 5021845-5024845 | KCNA5, Chr12: 5021845-5024845 |
| UNC93B1 | Chr11: 67526669-67529669 | UNC93B1, Chr11: 67526669-67529669 |
| OBSL1 | Chr2: 220142755-220145755 | OBSL1, Chr2: 220142755-220145755 |
| SOX30 | Chr5: 157010506-157013506 | SOX30, Chr5: 157010506-157013506 |
| LOC389151 | Chr3: 140220958-140223958 | LOC389151, Chr3: 140220958-140223958 |
| SH2D5 | Chr1: 20930220-20933220 | SH2D5, Chr1: 20930220-20933220 |
| KALRN | Chr3: 125784718-125787718 | KALRN, Chr3: 125784718-125787718 |
| SECTM1 | Chr17: 77883710-77886710 | SECTM1, Chr17: 77883710-77886710 |
| NDUFA4L2 | Chr12: 55919242-55922242 | NDUFA4L2, Chr12: 55919242-55922242 |
| MCF2L | Chr13: 112669257-112672257 | MCF2L, Chr13: 112669257-112672257 |
| DMRTB1 | Chr1: 53696159-53699159 | DMRTB1, Chr1: 53696159-53699159 |
| DNAJA4 | Chr15: 76342050-76345050 | DNAJA4, Chr15: 76342050-76345050 |
| COL23A1 | Chr5: 177948662-177951662 | COL23A1, Chr5: 177948662-177951662 |
| ZNF595 | Chr4: 41726-44726 | ZNF595, Chr4: 41726-44726 |
| ANKRD58 | Chr23: 118775103-118778103 | ANKRD58, Chr23: 118775103-118778103 |
| DLX6 | Chr7: 96471725-96474725 | DLX6, Chr7: 96471725-96474725 |
| GPR42P | Chr19: 40552601-40555601 | GPR42P, Chr19: 40552601-40555601 |
| ZNF718 | Chr4: 41776-44776 | ZNF718, Chr4: 41776-44776 |
| PEG3 | Chr19: 62042376-62045376 | PEG3, Chr19: 62042376-62045376 |
| MEST | Chr7: 129911781-129914781 | MEST, Chr7: 129911781-129914781 |
| GPR6 | Chr6: 110405490-110408490 | GPR6, Chr6: 110405490-110408490 |
| CADM3 | Chr1: 157406500-157409500 | CADM3, Chr1: 157406500-157409500 |
| LY6K | Chr8: 143777032-143780032 | LY6K, Chr8: 143777032-143780032 |
| CADM3 | Chr1: 157406537-157409537 | CADM3, Chr1: 157406537-157409537 |
| S1PR4 | Chr19: 3128265-3131265 | S1PR4, Chr19: 3128265-3131265 |
| PRKACG | Chr9: 70817328-70820328 | PRKACG, Chr9: 70817328-70820328 |
| WDR40B | Chr23: 125513015-125516015 | WDR40B, Chr23: 125513015-125516015 |
| AGXT2L2 | Chr5: 177590892-177593892 | AGXT2L2, Chr5: 177590892-177593892 |
| HIST3H3 | Chr1: 226678149-226681149 | HIST3H3, Chr1: 226678149-226681149 |
| PGCP | Chr8: 97725174-97728174 | PGCP, Chr8: 97725174-97728174 |
| CYP7B1 | Chr8: 65872402-65875402 | CYP7B1, Chr8: 65872402-65875402 |
| MST1R | Chr3: 49914810-49917810 | MST1R, Chr3: 49914810-49917810 |
| BCL9L | Chr11: 118285323-118288323 | BCL9L, Chr11: 118285323-118288323 |
| PRKCZ | Chr1: 1993445-1996445 | PRKCZ, Chr1: 1993445-1996445 |
| MYF5 | Chr12: 79633338-79636338 | MYF5, Chr12: 79633338-79636338 |
| ASZ1 | Chr7: 116853313-116856313 | ASZ1, Chr7: 116853313-116856313 |
| KRTAP4-2 | Chr17: 36586486-36589486 | KRTAP4-2, Chr17: 36586486-36589486 |
| RASL12 | Chr15: 63145941-63148941 | RASL12, Chr15: 63145941-63148941 |
| TSP50 | Chr3: 46732877-46735877 | TSP50, Chr3: 46732877-46735877 |
| TMEM204 | Chr16: 1522731-1525731 | TMEM204, Chr16: 1522731-1525731 |
| ADAM8 | Chr10: 134938862-134941862 | ADAM8, Chr10: 134938862-134941862 |
| C4orf39 | Chr4: 166096049-166099049 | C4orf39, Chr4: 166096049-166099049 |
| MRPS18A | Chr6: 43762006-43765006 | MRPS18A, Chr6: 43762006-43765006 |
| LSP1 | Chr11: 1847174-1850174 | LSP1, Chr11: 1847174-1850174 |
| TCEB3C | Chr18: 42808947-42811947 | TCEB3C, Chr18: 42808947-42811947 |
| GLTPD2 | Chr17: 4637493-4640493 | GLTPD2, Chr17: 4637493-4640493 |
| MGC4655 | Chr16: 65740903-65743903 | MGC4655, Chr16: 65740903-65743903 |
| PRPH | Chr12: 47973675-47976675 | PRPH, Chr12: 47973675-47976675 |
| BCR | Chr22: 21851051-21854051 | BCR, Chr22: 21851051-21854051 |
| DNMT3A | Chr2: 25417463-25420463 | DNMT3A, Chr2: 25417463-25420463 |
| DNMT1 | Chr19: 10165311-10168311 | DNMT1, Chr19: 10165311-10168311 |
| LOC390594 | Chr15: 63154706-63157706 | LOC390594, Chr15: 63154706-63157706 |
| ZNF354A | Chr5: 178088809-178091809 | ZNF354A, Chr5: 178088809-178091809 |
| KCNJ14 | Chr19: 53654817-53657817 | KCNJ14, Chr19: 53654817-53657817 |
| PHLDB3 | Chr19: 48699325-48702325 | PHLDB3, Chr19: 48699325-48702325 |
| TOR1AIP1 | Chr1: 178116542-178119542 | TOR1AIP1, Chr1: 178116542-178119542 |
| GBGT1 | Chr9: 135027622-135030622 | GBGT1, Chr9: 135027622-135030622 |
| ZBTB47 | Chr3: 42674377-42677377 | ZBTB47, Chr3: 42674377-42677377 |
| BAI1 | Chr8: 143540878-143543878 | BAI1, Chr8: 143540878-143543878 |
| TRPC7 | Chr5: 135719472-135722472 | TRPC7, Chr5: 135719472-135722472 |
| C1orf14 | Chr1: 181187676-181190676 | C1orf14, Chr1: 181187676-181190676 |
| ZIM2 | Chr19: 62042387-62045387 | ZIM2, Chr19: 62042387-62045387 |
| HIF3A | Chr19: 51497215-51500215 | HIF3A, Chr19: 51497215-51500215 |
| PRSS21 | Chr16: 2805664-2808664 | PRSS21, Chr16: 2805664-2808664 |
| MCCC1 | Chr3: 184298559-184301559 | MCCC1, Chr3: 184298559-184301559 |
| TEKT4 | Chr2: 94899458-94902458 | TEKT4, Chr2: 94899458-94902458 |
| PLEC1 | Chr8: 145095532-145098532 | PLEC1, Chr8: 145095532-145098532 |
| RHOXF1 | Chr23: 119132375-119135375 | RHOXF1, Chr23: 119132375-119135375 |
| COX8C | Chr14: 92881789-92884789 | COX8C, Chr14: 92881789-92884789 |
| MESP2 | Chr15: 88119092-88122092 | MESP2, Chr15: 88119092-88122092 |
| PDE6B | Chr4: 607872-610872 | PDE6B, Chr4: 607872-610872 |
| TNFRSF6B | Chr20: 61796964-61799964 | TNFRSF6B, Chr20: 61796964-61799964 |
| LIMS2 | Chr2: 128137083-128140083 | LIMS2, Chr2: 128137083-128140083 |
| TKTL2 | Chr4: 164612997-164615997 | TKTL2, Chr4: 164612997-164615997 |
| CSMD3 | Chr8: 114457058-114460058 | CSMD3, Chr8: 114457058-114460058 |
| CCT8L1 | Chr7: 151771756-151774756 | CCT8L1, Chr7: 151771756-151774756 |
| SLCO6A1 | Chr5: 101861119-101864119 | SLCO6A1, Chr5: 101861119-101864119 |
| SLC25A13 | Chr7: 95787895-95790895 | SLC25A13, Chr7: 95787895-95790895 |
| ABCC3 | Chr17: 46065726-46068726 | ABCC3, Chr17: 46065726-46068726 |
| NODAL | Chr10: 71869929-71872929 | NODAL, Chr10: 71869929-71872929 |
| UTS2R | Chr17: 77923989-77926989 | UTS2R, Chr17: 77923989-77926989 |
| PGAM2 | Chr7: 44070188-44073188 | PGAM2, Chr7: 44070188-44073188 |
| NLGN2 | Chr17: 7250725-7253725 | NLGN2, Chr17: 7250725-7253725 |
| COX7A1 | Chr19: 41334111-41337111 | COX7A1, Chr19: 41334111-41337111 |
| LOC643905 | Chr2: 240629572-240632572 | LOC643905, Chr2: 240629572-240632572 |
| TMC6 | Chr17: 73638583-73641583 | TMC6, Chr17: 73638583-73641583 |
| INSL3 | Chr19: 17791820-17794820 | INSL3, Chr19: 17791820-17794820 |
| KBTBD5 | Chr3: 42700514-42703514 | KBTBD5, Chr3: 42700514-42703514 |
| CNPY1 | Chr7: 155017800-155020800 | CNPY1, Chr7: 155017800-155020800 |
| DNAJC15 | Chr13: 42493861-42496861 | DNAJC15, Chr13: 42493861-42496861 |
| RTN1 | Chr14: 59262684-59265684 | RTN1, Chr14: 59262684-59265684 |
| BZRAP1 | Chr17: 53759651-53762651 | BZRAP1, Chr17: 53759651-53762651 |
| CALML5 | Chr10: 5530010-5533010 | CALML5, Chr10: 5530010-5533010 |
| GPR68 | Chr14: 90788477-90791477 | GPR68, Chr14: 90788477-90791477 |
| C15orf2 | Chr15: 22470133-22473133 | C15orf2, Chr15: 22470133-22473133 |
| HNF1A | Chr12: 119899431-119902431 | HNF1A, Chr12: 119899431-119902431 |
| SRMS | Chr20: 61647801-61650801 | SRMS, Chr20: 61647801-61650801 |
| NHP2L1 | Chr22: 40407002-40410002 | NHP2L1, Chr22: 40407002-40410002 |
| DNAJB3 | Chr2: 234315900-234318900 | DNAJB3, Chr2: 234315900-234318900 |
| C20orf134 | Chr20: 31716464-31719464 | C20orf134, Chr20: 31716464-31719464 |
| SLC13A4 | Chr7: 135061973-135064973 | SLC13A4, Chr7: 135061973-135064973 |
| KCNV2 | Chr9: 2706025-2709025 | KCNV2, Chr9: 2706025-2709025 |
| C20orf135 | Chr20: 61961509-61964509 | C20orf135, Chr20: 61961509-61964509 |
| C1QC | Chr1: 22841204-22844204 | C1QC, Chr1: 22841204-22844204 |
| SPON2 | Chr4: 1155141-1158141 | SPON2, Chr4: 1155141-1158141 |
| FIGNL2 | Chr12: 50500975-50503975 | FIGNL2, Chr12: 50500975-50503975 |
| PTGDS | Chr9: 138990276-138993276 | PTGDS, Chr9: 138990276-138993276 |
| COX6A2 | Chr16: 31345722-31348722 | COX6A2, Chr16: 31345722-31348722 |
| HTRA4 | Chr8: 38949353-38952353 | HTRA4, Chr8: 38949353-38952353 |
| ACTL7B | Chr9: 110656531-110659531 | ACTL7B, Chr9: 110656531-110659531 |
| SOX2 | Chr3: 182910915-182913915 | SOX2, Chr3: 182910915-182913915 |
| PRIC285 | Chr20: 61668051-61671051 | PRIC285, Chr20: 61668051-61671051 |
| CLDN4 | Chr7: 72881628-72884628 | CLDN4, Chr7: 72881628-72884628 |
| BIRC8 | Chr19: 58485187-58488187 | BIRC8, Chr19: 58485187-58488187 |
| CALML3 | Chr10: 5555423-5558423 | CALML3, Chr10: 5555423-5558423 |
| NPBWR2 | Chr20: 62207128-62210128 | NPBWR2, Chr20: 62207128-62210128 |
| LOC389257 | Chr5: 243125-246125 | LOC389257, Chr5: 243125-246125 |
| WDR13 | Chr23: 48339344-48342344 | WDR13, Chr23: 48339344-48342344 |
| ADAD1 | Chr4: 123518117-123521117 | ADAD1, Chr4: 123518117-123521117 |
| LOC345222 | Chr4: 3219064-3222064 | LOC345222, Chr4: 3219064-3222064 |
| PNCK | Chr23: 152591474-152594474 | PNCK, Chr23: 152591474-152594474 |
| AMIGO3 | Chr3: 49730627-49733627 | AMIGO3, Chr3: 49730627-49733627 |
| MGAT3 | Chr22: 38211674-38214674 | MGAT3, Chr22: 38211674-38214674 |
| CYP2E1 | Chr10: 135189356-135192356 | CYP2E1, Chr10: 135189356-135192356 |
| GNAS | Chr20: 56846689-56849689 | GNAS, Chr20: 56846689-56849689 |
| FAM176B | Chr1: 36560842-36563842 | FAM176B, Chr1: 36560842-36563842 |
| RTN4 | Chr2: 55129738-55132738 | RTN4, Chr2: 55129738-55132738 |
| GPR62 | Chr3: 51962869-51965869 | GPR62, Chr3: 51962869-51965869 |
| PTCHD3 | Chr10: 27741803-27744803 | PTCHD3, Chr10: 27741803-27744803 |
| KIAA1975 | Chr10: 88718977-88721977 | KIAA1975, Chr10: 88718977-88721977 |
| TCEB3B | Chr18: 42814486-42817486 | TCEB3B, Chr18: 42814486-42817486 |
| GPR45 | Chr2: 105223131-105226131 | GPR45, Chr2: 105223131-105226131 |
| TP53 | Chr17: 7518036-7521036 | TP53, Chr17: 7518036-7521036 |
| ALPK3 | Chr15: 83159414-83162414 | ALPK3, Chr15: 83159414-83162414 |
| CXorf20 | Chr23: 18147445-18150445 | CXorf20, Chr23: 18147445-18150445 |
| ZNRF4 | Chr19: 5404925-5407925 | ZNRF4, Chr19: 5404925-5407925 |
| PRDM8 | Chr4: 81336180-81339180 | PRDM8, Chr4: 81336180-81339180 |
| LDHD | Chr16: 73706666-73709666 | LDHD, Chr16: 73706666-73709666 |
| REPIN1 | Chr7: 149697690-149700690 | REPIN1, Chr7: 149697690-149700690 |
| PRLHR | Chr10: 120343650-120346650 | PRLHR, Chr10: 120343650-120346650 |
| C9orf167 | Chr9: 139290600-139293600 | C9orf167, Chr9: 139290600-139293600 |
| BEGAIN | Chr14: 100104384-100107384 | BEGAIN, Chr14: 100104384-100107384 |
| BBC3 | Chr19: 52426363-52429363 | BBC3, Chr19: 52426363-52429363 |
| TNFAIP2 | Chr14: 102660916-102663916 | TNFAIP2, Chr14: 102660916-102663916 |
| H1FNT | Chr12: 47007529-47010529 | H1FNT, Chr12: 47007529-47010529 |
| SCNN1D | Chr1: 1205938-1208938 | SCNN1D, Chr1: 1205938-1208938 |
| KLHDC7B | Chr22: 49331827-49334827 | KLHDC7B, Chr22: 49331827-49334827 |
| HOXD10 | Chr2: 176688237-176691237 | HOXD10, Chr2: 176688237-176691237 |
| ISLR | Chr15: 72252457-72255457 | ISLR, Chr15: 72252457-72255457 |
| FERMT3 | Chr11: 63729281-63732281 | FERMT3, Chr11: 63729281-63732281 |
| FERMT3 | Chr11: 63729288-63732288 | FERMT3, Chr11: 63729288-63732288 |
| SPON2 | Chr4: 1155480-1158480 | SPON2, Chr4: 1155480-1158480 |
| TMC8 | Chr17: 73636953-73639953 | TMC8, Chr17: 73636953-73639953 |
| FAM83G | Chr17: 18847285-18850285 | FAM83G, Chr17: 18847285-18850285 |
| MAP2K3 | Chr17: 21133873-21136873 | MAP2K3, Chr17: 21133873-21136873 |
| TRPC3 | Chr4: 123072561-123075561 | TRPC3, Chr4: 123072561-123075561 |
| PLXNA1 | Chr3: 128188691-128191691 | PLXNA1, Chr3: 128188691-128191691 |
| TPSG1 | Chr16: 1213755-1216755 | TPSG1, Chr16: 1213755-1216755 |
| CCDC17 | Chr1: 45860816-45863816 | CCDC17, Chr1: 45860816-45863816 |
| CCDC36 | Chr3: 49209364-49212364 | CCDC36, Chr3: 49209364-49212364 |
| CLEC4G | Chr19: 7701505-7704505 | CLEC4G, Chr19: 7701505-7704505 |
| NOXO1 | Chr16: 1969685-1972685 | NOXO1, Chr16: 1969685-1972685 |
| PNMAL2 | Chr19: 51689509-51692509 | PNMAL2, Chr19: 51689509-51692509 |
| LRFN1 | Chr19: 44496316-44499316 | LRFN1, Chr19: 44496316-44499316 |
| SOX15 | Chr17: 7432712-7435712 | SOX15, Chr17: 7432712-7435712 |
| FAM110A | Chr20: 771784-774784 | FAM110A, Chr20: 771784-774784 |
| ITGA11 | Chr15: 66510046-66513046 | ITGA11, Chr15: 66510046-66513046 |
| SOX10 | Chr22: 36708985-36711985 | SOX10, Chr22: 36708985-36711985 |
| LOC340602 | Chr23: 51165006-51168006 | LOC340602, Chr23: 51165006-51168006 |
| CCDC124 | Chr19: 17905404-17908404 | CCDC124, Chr19: 17905404-17908404 |
| MAFG | Chr17: 77473201-77476201 | MAFG, Chr17: 77473201-77476201 |
| MAB21L2 | Chr4: 151721252-151724252 | MAB21L2, Chr4: 151721252-151724252 |
| MAGEE1 | Chr23: 75563020-75566020 | MAGEE1, Chr23: 75563020-75566020 |
| GPR4 | Chr19: 50786057-50789057 | GPR4, Chr19: 50786057-50789057 |
| EPS8L1 | Chr19: 60282071-60285071 | EPS8L1, Chr19: 60282071-60285071 |
| CLDND2 | Chr19: 56562569-56565569 | CLDND2, Chr19: 56562569-56565569 |
| KIAA1688 | Chr8: 145808196-145811196 | KIAA1688, Chr8: 145808196-145811196 |
| NOXO1 | Chr16: 1969941-1972941 | NOXO1, Chr16: 1969941-1972941 |
| PLEKHG4 | Chr16: 65868076-65871076 | PLEKHG4, Chr16: 65868076-65871076 |
| TCTEX1D4 | Chr1: 45044044-45047044 | TCTEX1D4, Chr1: 45044044-45047044 |
| ZNF831 | Chr20: 57197969-57200969 | ZNF831, Chr20: 57197969-57200969 |
| F2RL3 | Chr19: 16859325-16862325 | F2RL3, Chr19: 16859325-16862325 |
| BRD1 | Chr22: 48602956-48605956 | BRD1, Chr22: 48602956-48605956 |
| C6orf27 | Chr6: 31851587-31854587 | C6orf27, Chr6: 31851587-31854587 |
| ZNF835 | Chr19: 61874558-61877558 | ZNF835, Chr19: 61874558-61877558 |
| LCK | Chr1: 32510798-32513798 | LCK, Chr1: 32510798-32513798 |
| AHRR | Chr5: 355790-358790 | AHRR, Chr5: 355790-358790 |
| MATN4 | Chr20: 43366145-43369145 | MATN4, Chr20: 43366145-43369145 |
| PAX8 | Chr2: 113751468-113754468 | PAX8, Chr2: 113751468-113754468 |
| CABP4 | Chr11: 66977893-66980893 | CABP4, Chr11: 66977893-66980893 |
| MXRA8 | Chr1: 1282278-1285278 | MXRA8, Chr1: 1282278-1285278 |
| C17orf55 | Chr17: 76896143-76899143 | C17orf55, Chr17: 76896143-76899143 |
| PCDHA1 | Chr5: 140144559-140147559 | PCDHA1, Chr5: 140144559-140147559 |
| CNGA4 | Chr11: 6215405-6218405 | CNGA4, Chr11: 6215405-6218405 |
| GPR142 | Chr17: 69873739-69876739 | GPR142, Chr17: 69873739-69876739 |
| HOXB2 | Chr17: 43975892-43978892 | HOXB2, Chr17: 43975892-43978892 |
| CYP2D6 | Chr22: 40855327-40858327 | CYP2D6, Chr22: 40855327-40858327 |
| TBC1D10C | Chr11: 66926487-66929487 | TBC1D10C, Chr11: 66926487-66929487 |
| PTGIR | Chr19: 51818694-51821694 | PTGIR, Chr19: 51818694-51821694 |
| TCF23 | Chr2: 27224005-27227005 | TCF23, Chr2: 27224005-27227005 |
| PRRT1 | Chr6: 32226198-32229198 | PRRT1, Chr6: 32226198-32229198 |
| DNAH1 | Chr3: 52324059-52327059 | DNAH1, Chr3: 52324059-52327059 |
| GJA4 | Chr1: 35029685-35032685 | GJA4, Chr1: 35029685-35032685 |
| MGAT4B | Chr5: 179161177-179164177 | MGAT4B, Chr5: 179161177-179164177 |
| PTGER1 | Chr19: 14445674-14448674 | PTGER1, Chr19: 14445674-14448674 |
| TRIP6 | Chr7: 100301385-100304385 | TRIP6, Chr7: 100301385-100304385 |
| WNT10B | Chr12: 47650310-47653310 | WNT10B, Chr12: 47650310-47653310 |
| ISLR | Chr15: 72251639-72254639 | ISLR, Chr15: 72251639-72254639 |
| LOC87769 | Chr13: 99982498-99985498 | LOC87769, Chr13: 99982498-99985498 |
| SLC45A4 | Chr8: 142306355-142309355 | SLC45A4, Chr8: 142306355-142309355 |
| GPT | Chr8: 145698772-145701772 | GPT, Chr8: 145698772-145701772 |
| ZNF579 | Chr19: 60782523-60785523 | ZNF579, Chr19: 60782523-60785523 |
| JPH4 | Chr14: 23116334-23119334 | JPH4, Chr14: 23116334-23119334 |
| AVP | Chr20: 3011870-3014870 | AVP, Chr20: 3011870-3014870 |
| PKP3 | Chr11: 382716-385716 | PKP3, Chr11: 382716-385716 |
| FZR1 | Chr19: 3472453-3475453 | FZR1, Chr19: 3472453-3475453 |
| ELFN1 | Chr7: 1749132-1752132 | ELFN1, Chr7: 1749132-1752132 |
| CLIC3 | Chr9: 139009345-139012345 | CLIC3, Chr9: 139009345-139012345 |
| KLK5 | Chr19: 56146656-56149656 | KLK5, Chr19: 56146656-56149656 |
| LRRN4 | Chr20: 5981194-5984194 | LRRN4, Chr20: 5981194-5984194 |
| CCR10 | Chr17: 38085871-38088871 | CCR10, Chr17: 38085871-38088871 |
| MCOLN1 | Chr19: 7492011-7495011 | MCOLN1, Chr19: 7492011-7495011 |
| SMTNL2 | Chr17: 4432524-4435524 | SMTNL2, Chr17: 4432524-4435524 |
| BLOC1S3 | Chr19: 50372342-50375342 | BLOC1S3, Chr19: 50372342-50375342 |
| TRAPPC2L | Chr16: 87449508-87452508 | TRAPPC2L, Chr16: 87449508-87452508 |
| SLMO1 | Chr18: 12396427-12399427 | SLMO1, Chr18: 12396427-12399427 |
| CTBP1 | Chr4: 1231408-1234408 | CTBP1, Chr4: 1231408-1234408 |
| CEBPB | Chr20: 48239282-48242282 | CEBPB, Chr20: 48239282-48242282 |
| UHRF2 | Chr9: 6401650-6404650 | UHRF2, Chr9: 6401650-6404650 |
| ADARB1 | Chr21: 45317442-45320442 | ADARB1, Chr21: 45317442-45320442 |
| PACS2 | Chr14: 104850625-104853625 | PACS2, Chr14: 104850625-104853625 |
| PCYT2 | Chr17: 77461086-77464086 | PCYT2, Chr17: 77461086-77464086 |
| DRAP1 | Chr11: 65441803-65444803 | DRAP1, Chr11: 65441803-65444803 |
| COBRA1 | Chr9: 139268079-139271079 | COBRA1, Chr9: 139268079-139271079 |
| NCAPH2 | Chr22: 49292010-49295010 | NCAPH2, Chr22: 49292010-49295010 |
| CDC34 | Chr19: 481232-484232 | CDC34, Chr19: 481232-484232 |
| PUS1 | Chr12: 130978241-130981241 | PUS1, Chr12: 130978241-130981241 |
| CCDC137 | Chr17: 77242665-77245665 | CCDC137, Chr17: 77242665-77245665 |
| PPFIA3 | Chr19: 54312974-54315974 | PPFIA3, Chr19: 54312974-54315974 |
| HSPA4L | Chr4: 128921402-128924402 | HSPA4L, Chr4: 128921402-128924402 |
| DNASE1L1 | Chr23: 153292121-153295121 | DNASE1L1, Chr23: 153292121-153295121 |
| ANAPC2 | Chr9: 139201378-139204378 | ANAPC2, Chr9: 139201378-139204378 |
| ANKRD9 | Chr14: 102044381-102047381 | ANKRD9, Chr14: 102044381-102047381 |
| MYEOV2 | Chr2: 240722937-240725937 | MYEOV2, Chr2: 240722937-240725937 |
| ASB1 | Chr2: 238998864-239001864 | ASB1, Chr2: 238998864-239001864 |
| TSPAN4 | Chr11: 831440-834440 | TSPAN4, Chr11: 831440-834440 |
| HNRNPH3 | Chr10: 69760273-69763273 | HNRNPH3, Chr10: 69760273-69763273 |
| ARL15 | Chr5: 53640660-53643660 | ARL15, Chr5: 53640660-53643660 |
| CDKN1C | Chr11: 2862071-2865071 | CDKN1C, Chr11: 2862071-2865071 |
| FGF3 | Chr11: 69341629-69344629 | FGF3, Chr11: 69341629-69344629 |
| WNT9A | Chr1: 226200722-226203722 | WNT9A, Chr1: 226200722-226203722 |
| FBXL6 | Chr8: 145551440-145554440 | FBXL6, Chr8: 145551440-145554440 |
| DBP | Chr19: 53830951-53833951 | DBP, Chr19: 53830951-53833951 |
| C9orf69 | Chr9: 138149052-138152052 | C9orf69, Chr9: 138149052-138152052 |
| PDDC1 | Chr11: 765987-768987 | PDDC1, Chr11: 765987-768987 |
| SLCO4A1 | Chr20: 60742741-60745741 | SLCO4A1, Chr20: 60742741-60745741 |
| C1orf159 | Chr1: 1040099-1043099 | C1orf159, Chr1: 1040099-1043099 |
| TSPAN4 | Chr11: 831323-834323 | TSPAN4, Chr11: 831323-834323 |
| MCM5 | Chr22: 34124615-34127615 | MCM5, Chr22: 34124615-34127615 |
| PPP1R8 | Chr1: 28028379-28031379 | PPP1R8, Chr1: 28028379-28031379 |
| CLPTM1L | Chr5: 1396502-1399502 | CLPTM1L, Chr5: 1396502-1399502 |
| ZNF696 | Chr8: 144443433-144446433 | ZNF696, Chr8: 144443433-144446433 |
| PEX10 | Chr1: 2332370-2335370 | PEX10, Chr1: 2332370-2335370 |
| LIG1 | Chr19: 53363872-53366872 | LIG1, Chr19: 53363872-53366872 |
| GLTPD1 | Chr1: 1248505-1251505 | GLTPD1, Chr1: 1248505-1251505 |
| FAM3A | Chr23: 153396067-153399067 | FAM3A, Chr23: 153396067-153399067 |
| PRX | Chr19: 45609611-45612611 | PRX, Chr19: 45609611-45612611 |
| PIM3 | Chr22: 48738664-48741664 | PIM3, Chr22: 48738664-48741664 |
| LASS1 | Chr19: 18866453-18869453 | LASS1, Chr19: 18866453-18869453 |
| EPHB2 | Chr1: 22908417-22911417 | EPHB2, Chr1: 22908417-22911417 |
| DEGS2 | Chr14: 99694212-99697212 | DEGS2, Chr14: 99694212-99697212 |
| SLC2A4RG | Chr20: 61840154-61843154 | SLC2A4RG, Chr20: 61840154-61843154 |
| RBPMS2 | Chr15: 62853323-62856323 | RBPMS2, Chr15: 62853323-62856323 |
| CABP7 | Chr22: 28444843-28447843 | CABP7, Chr22: 28444843-28447843 |
| ADCK2 | Chr7: 140017921-140020921 | ADCK2, Chr7: 140017921-140020921 |
| APRT | Chr16: 87404343-87407343 | APRT, Chr16: 87404343-87407343 |
| AHNAK2 | Chr14: 104514239-104517239 | AHNAK2, Chr14: 104514239-104517239 |
| TUBGCP6 | Chr22: 49024027-49027027 | TUBGCP6, Chr22: 49024027-49027027 |
| C17orf70 | Chr17: 77128348-77131348 | C17orf70, Chr17: 77128348-77131348 |
| C17orf70 | Chr17: 77128368-77131368 | C17orf70, Chr17: 77128368-77131368 |
| ARHGEF16 | Chr1: 3359506-3362506 | ARHGEF16, Chr1: 3359506-3362506 |
| TRIP13 | Chr5: 944503-947503 | TRIP13, Chr5: 944503-947503 |
| ANKRD20A4 | Chr9: 68670300-68673300 | ANKRD20A4, Chr9: 68670300-68673300 |
| NUMBL | Chr19: 45886896-45889896 | NUMBL, Chr19: 45886896-45889896 |
| LLGL1 | Chr17: 18068160-18071160 | LLGL1, Chr17: 18068160-18071160 |
| LPCAT1 | Chr5: 1575576-1578576 | LPCAT1, Chr5: 1575576-1578576 |
| COL6A2 | Chr21: 46340960-46343960 | COL6A2, Chr21: 46340960-46343960 |
| TFDP1 | Chr13: 113285556-113288556 | TFDP1, Chr13: 113285556-113288556 |
| TBCB | Chr19: 41296227-41299227 | TBCB, Chr19: 41296227-41299227 |
| POLRMT | Chr19: 583068-586068 | POLRMT, Chr19: 583068-586068 |
| DEK | Chr6: 18371278-18374278 | DEK, Chr6: 18371278-18374278 |
| TCEB2 | Chr16: 2765798-2768798 | TCEB2, Chr16: 2765798-2768798 |
| LOC116349 | Chr5: 494710-497710 | LOC116349, Chr5: 494710-497710 |
| CERK | Chr22: 45511316-45514316 | CERK, Chr22: 45511316-45514316 |
| AP3B2 | Chr15: 81174189-81177189 | AP3B2, Chr15: 81174189-81177189 |
| UBE2J2 | Chr1: 1197597-1200597 | UBE2J2, Chr1: 1197597-1200597 |
| RYK | Chr3: 135450776-135453776 | RYK, Chr3: 135450776-135453776 |
| UBL4A | Chr23: 153366626-153369626 | UBL4A, Chr23: 153366626-153369626 |
| NINJ1 | Chr9: 94934891-94937891 | NINJ1, Chr9: 94934891-94937891 |
| PDCD6 | Chr5: 323235-326235 | PDCD6, Chr5: 323235-326235 |
| RBM38 | Chr20: 55398369-55401369 | RBM38, Chr20: 55398369-55401369 |
| UNC5B | Chr10: 72640854-72643854 | UNC5B, Chr10: 72640854-72643854 |
| ZNF566 | Chr19: 41670677-41673677 | ZNF566, Chr19: 41670677-41673677 |
| MGC3207 | Chr19: 13734836-13737836 | MGC3207, Chr19: 13734836-13737836 |
| ABCA2 | Chr9: 139041061-139044061 | ABCA2, Chr9: 139041061-139044061 |
| KCNQ1 | Chr11: 2421296-2424296 | KCNQ1, Chr11: 2421296-2424296 |
| GRINA | Chr8: 145134713-145137713 | GRINA, Chr8: 145134713-145137713 |
| SORCS2 | Chr4: 7243774-7246774 | SORCS2, Chr4: 7243774-7246774 |
| KIAA0146 | Chr8: 48334594-48337594 | KIAA0146, Chr8: 48334594-48337594 |
| MRPL23 | Chr11: 1923613-1926613 | MRPL23, Chr11: 1923613-1926613 |
| SUSD3 | Chr9: 94859309-94862309 | SUSD3, Chr9: 94859309-94862309 |
| MKI67IP | Chr2: 122209438-122212438 | MKI67IP, Chr2: 122209438-122212438 |
| PITRM1 | Chr10: 3203503-3206503 | PITRM1, Chr10: 3203503-3206503 |
| ZNF256 | Chr19: 63149389-63152389 | ZNF256, Chr19: 63149389-63152389 |
| ANKRD20A3 | Chr9: 42356798-42359798 | ANKRD20A3, Chr9: 42356798-42359798 |
| PLEKHG5 | Chr1: 6478571-6481571 | PLEKHG5, Chr1: 6478571-6481571 |
| PDLIM2 | Chr8: 22491087-22494087 | PDLIM2, Chr8: 22491087-22494087 |
| ATP8B3 | Chr19: 1761770-1764770 | ATP8B3, Chr19: 1761770-1764770 |
| ENTPD6 | Chr20: 25122838-25125838 | ENTPD6, Chr20: 25122838-25125838 |
| CAMK2G | Chr10: 75302844-75305844 | CAMK2G, Chr10: 75302844-75305844 |
| CAMK2G | Chr10: 75302849-75305849 | CAMK2G, Chr10: 75302849-75305849 |
| UHRF1 | Chr19: 4859009-4862009 | UHRF1, Chr19: 4859009-4862009 |
| KCNQ2 | Chr20: 61572937-61575937 | KCNQ2, Chr20: 61572937-61575937 |
| PRKX | Chr23: 3640161-3643161 | PRKX, Chr23: 3640161-3643161 |
| COL5A1 | Chr9: 136671972-136674972 | COL5A1, Chr9: 136671972-136674972 |
| GIT1 | Chr17: 24939236-24942236 | GIT1, Chr17: 24939236-24942236 |
| B3GNT7 | Chr2: 231967078-231970078 | B3GNT7, Chr2: 231967078-231970078 |
| MIF4GD | Chr17: 70777398-70780398 | MIF4GD, Chr17: 70777398-70780398 |
| ABCB9 | Chr12: 122024205-122027205 | ABCB9, Chr12: 122024205-122027205 |
| DGCR6L | Chr22: 18686108-18689108 | DGCR6L, Chr22: 18686108-18689108 |
| GPS2 | Chr17: 7157882-7160882 | GPS2, Chr17: 7157882-7160882 |
| MYO1C | Chr17: 1340222-1343222 | MYO1C, Chr17: 1340222-1343222 |
| B4GALT2 | Chr1: 44215952-44218952 | B4GALT2, Chr1: 44215952-44218952 |
| MAPK12 | Chr22: 49040716-49043716 | MAPK12, Chr22: 49040716-49043716 |
| OSBPL2 | Chr20: 60245474-60248474 | OSBPL2, Chr20: 60245474-60248474 |
| GAMT | Chr19: 1351052-1354052 | GAMT, Chr19: 1351052-1354052 |
| COL9A3 | Chr20: 60917358-60920358 | COL9A3, Chr20: 60917358-60920358 |
| CD151 | Chr11: 821451-824451 | CD151, Chr11: 821451-824451 |
| SLC9A3R2 | Chr16: 2015388-2018388 | SLC9A3R2, Chr16: 2015388-2018388 |
| MTA1 | Chr14: 104955730-104958730 | MTA1, Chr14: 104955730-104958730 |
| FAM96B | Chr16: 65524304-65527304 | FAM96B, Chr16: 65524304-65527304 |
| SFRS16 | Chr19: 50232637-50235637 | SFRS16, Chr19: 50232637-50235637 |
| ADPRHL2 | Chr1: 36325572-36328572 | ADPRHL2, Chr1: 36325572-36328572 |
| EEF1A2 | Chr20: 61599449-61602449 | EEF1A2, Chr20: 61599449-61602449 |
| EPS8L2 | Chr11: 694622-697622 | EPS8L2, Chr11: 694622-697622 |
| NUDT14 | Chr14: 104717185-104720185 | NUDT14, Chr14: 104717185-104720185 |
| SSH3 | Chr11: 66826076-66829076 | SSH3, Chr11: 66826076-66829076 |
| ANXA11 | Chr10: 81953808-81956808 | ANXA11, Chr10: 81953808-81956808 |
| RPE | Chr2: 210574096-210577096 | RPE, Chr2: 210574096-210577096 |
| RPE | Chr2: 210574125-210577125 | RPE, Chr2: 210574125-210577125 |
| PRDX4 | Chr23: 23594065-23597065 | PRDX4, Chr23: 23594065-23597065 |
| MGC50273 | Chr2: 132274204-132277204 | MGC50273, Chr2: 132274204-132277204 |
| FSTL3 | Chr19: 625888-628888 | FSTL3, Chr19: 625888-628888 |
| PGF | Chr14: 74490544-74493544 | PGF, Chr14: 74490544-74493544 |
| MEGF11 | Chr15: 64331629-64334629 | MEGF11, Chr15: 64331629-64334629 |
| HTRA3 | Chr4: 8320891-8323891 | HTRA3, Chr4: 8320891-8323891 |
| LRP8 | Chr1: 53564814-53567814 | LRP8, Chr1: 53564814-53567814 |
| SIGIRR | Chr11: 405897-408897 | SIGIRR, Chr11: 405897-408897 |
| EEF1G | Chr11: 62096536-62099536 | EEF1G, Chr11: 62096536-62099536 |
| DPP7 | Chr9: 139127516-139130516 | DPP7, Chr9: 139127516-139130516 |
| DUSP2 | Chr2: 96173406-96176406 | DUSP2, Chr2: 96173406-96176406 |
| SQSTM1 | Chr5: 179178947-179181947 | SQSTM1, Chr5: 179178947-179181947 |
| PANK4 | Chr1: 2446395-2449395 | PANK4, Chr1: 2446395-2449395 |
| GANAB | Chr11: 62169180-62172180 | GANAB, Chr11: 62169180-62172180 |
| EXOC2 | Chr6: 636609-639609 | EXOC2, Chr6: 636609-639609 |
| RTN4R | Chr22: 18634316-18637316 | RTN4R, Chr22: 18634316-18637316 |
| HYAL2 | Chr3: 50332403-50335403 | HYAL2, Chr3: 50332403-50335403 |
| C10orf116 | Chr10: 88716667-88719667 | C10orf116, Chr10: 88716667-88719667 |
| MAP4K2 | Chr11: 64325789-64328789 | MAP4K2, Chr11: 64325789-64328789 |
| SARS2 | Chr19: 44111876-44114876 | SARS2, Chr19: 44111876-44114876 |
| CDK10 | Chr16: 88279076-88282076 | CDK10, Chr16: 88279076-88282076 |
| CDK10 | Chr16: 88279078-88282078 | CDK10, Chr16: 88279078-88282078 |
| DSCAM | Chr21: 41139409-41142409 | DSCAM, Chr21: 41139409-41142409 |
| TTC7B | Chr14: 90351014-90354014 | TTC7B, Chr14: 90351014-90354014 |
| OSBPL5 | Chr11: 3141616-3144616 | OSBPL5, Chr11: 3141616-3144616 |
| CD2BP2 | Chr16: 30272628-30275628 | CD2BP2, Chr16: 30272628-30275628 |
| MTG1 | Chr10: 135056110-135059110 | MTG1, Chr10: 135056110-135059110 |
| B4GALT4 | Chr3: 120440942-120443942 | B4GALT4, Chr3: 120440942-120443942 |
| TMC6 | Chr17: 73634956-73637956 | TMC6, Chr17: 73634956-73637956 |
| LGI3 | Chr8: 22068789-22071789 | LGI3, Chr8: 22068789-22071789 |
| C14orf94 | Chr14: 22494660-22497660 | C14orf94, Chr14: 22494660-22497660 |
| RPS19BP1 | Chr22: 38257306-38260306 | RPS19BP1, Chr22: 38257306-38260306 |
| ZNF502 | Chr3: 44727641-44730641 | ZNF502, Chr3: 44727641-44730641 |
| LRP8 | Chr1: 53564909-53567909 | LRP8, Chr1: 53564909-53567909 |
| TGFB1 | Chr19: 46550156-46553156 | TGFB1, Chr19: 46550156-46553156 |
| CTXN1 | Chr19: 7895551-7898551 | CTXN1, Chr19: 7895551-7898551 |
| ARL6IP4 | Chr12: 122029332-122032332 | ARL6IP4, Chr12: 122029332-122032332 |
| EXOSC6 | Chr16: 68841834-68844834 | EXOSC6, Chr16: 68841834-68844834 |
| DDX54 | Chr12: 112106167-112109167 | DDX54, Chr12: 112106167-112109167 |
| NPDC1 | Chr9: 139058976-139061976 | NPDC1, Chr9: 139058976-139061976 |
| CENPL | Chr1: 172059149-172062149 | CENPL, Chr1: 172059149-172062149 |
| MMD2 | Chr7: 4963870-4966870 | MMD2, Chr7: 4963870-4966870 |
| C1orf142 | Chr1: 225987819-225990819 | C1orf142, Chr1: 225987819-225990819 |
| C12orf57 | Chr12: 6921963-6924963 | C12orf57, Chr12: 6921963-6924963 |
| TMEM120A | Chr7: 75460389-75463389 | TMEM120A, Chr7: 75460389-75463389 |
| CNIH | Chr14: 53976398-53979398 | CNIH, Chr14: 53976398-53979398 |
| SIRT7 | Chr17: 77467832-77470832 | SIRT7, Chr17: 77467832-77470832 |
| COL2A1 | Chr12: 46683052-46686052 | COL2A1, Chr12: 46683052-46686052 |
| SAMD12 | Chr8: 119701865-119704865 | SAMD12, Chr8: 119701865-119704865 |
| NECAB3 | Chr20: 31724425-31727425 | NECAB3, Chr20: 31724425-31727425 |
| ZNF414 | Chr19: 8483532-8486532 | ZNF414, Chr19: 8483532-8486532 |
| INTS8 | Chr8: 95903209-95906209 | INTS8, Chr8: 95903209-95906209 |
| CILP2 | Chr19: 19508573-19511573 | CILP2, Chr19: 19508573-19511573 |
| C1QTNF5 | Chr11: 118715291-118718291 | C1QTNF5, Chr11: 118715291-118718291 |
| GABRA5 | Chr15: 24661650-24664650 | GABRA5, Chr15: 24661650-24664650 |
| MDK | Chr11: 46357693-46360693 | MDK, Chr11: 46357693-46360693 |
| NAGLU | Chr17: 37939976-37942976 | NAGLU, Chr17: 37939976-37942976 |
| MGAT1 | Chr5: 180161154-180164154 | MGAT1, Chr5: 180161154-180164154 |
| C2orf3 | Chr2: 75790330-75793330 | C2orf3, Chr2: 75790330-75793330 |
| MREG | Chr2: 216585091-216588091 | MREG, Chr2: 216585091-216588091 |
| C20orf200 | Chr20: 60557676-60560676 | C20orf200, Chr20: 60557676-60560676 |
| PLEKHN1 | Chr1: 890239-893239 | PLEKHN1, Chr1: 890239-893239 |
| LIF | Chr22: 28971248-28974248 | LIF, Chr22: 28971248-28974248 |
| C9orf139 | Chr9: 139040236-139043236 | C9orf139, Chr9: 139040236-139043236 |
| ABCA2 | Chr9: 139041695-139044695 | ABCA2, Chr9: 139041695-139044695 |
| ADNP2 | Chr18: 75966402-75969402 | ADNP2, Chr18: 75966402-75969402 |
| PILRA | Chr7: 99807503-99810503 | PILRA, Chr7: 99807503-99810503 |
| HOXA2 | Chr7: 27107419-27110419 | HOXA2, Chr7: 27107419-27110419 |
| TM7SF2 | Chr11: 64634416-64637416 | TM7SF2, Chr11: 64634416-64637416 |
| CGB | Chr19: 54217905-54220905 | CGB, Chr19: 54217905-54220905 |
| C6orf153 | Chr6: 43095862-43098862 | C6orf153, Chr6: 43095862-43098862 |
| C20orf166 | Chr20: 60556604-60559604 | C20orf166, Chr20: 60556604-60559604 |
| NKX6-3 | Chr8: 41622535-41625535 | NKX6-3, Chr8: 41622535-41625535 |
| HORMAD1 | Chr1: 148958476-148961476 | HORMAD1, Chr1: 148958476-148961476 |
| PRKCZ | Chr1: 2024514-2027514 | PRKCZ, Chr1: 2024514-2027514 |
| C3orf24 | Chr3: 10123415-10126415 | C3orf24, Chr3: 10123415-10126415 |
| FGR | Chr1: 27832814-27835814 | FGR, Chr1: 27832814-27835814 |
| PLEC1 | Chr8: 145089393-145092393 | PLEC1, Chr8: 145089393-145092393 |
| MPG | Chr16: 65517-68517 | MPG, Chr16: 65517-68517 |
| NEUROD2 | Chr17: 35016201-35019201 | NEUROD2, Chr17: 35016201-35019201 |
| MYO1C | Chr17: 1341251-1344251 | MYO1C, Chr17: 1341251-1344251 |
| LGALS4 | Chr19: 43993922-43996922 | LGALS4, Chr19: 43993922-43996922 |
| PAGE2B | Chr23: 55116728-55119728 | PAGE2B, Chr23: 55116728-55119728 |
| SPIN2A | Chr23: 57179283-57182283 | SPIN2A, Chr23: 57179283-57182283 |
| PILRB | Chr7: 99792061-99795061 | PILRB, Chr7: 99792061-99795061 |
| PLSCR2 | Chr3: 147694912-147697912 | PLSCR2, Chr3: 147694912-147697912 |
| DMKN | Chr19: 40691752-40694752 | DMKN, Chr19: 40691752-40694752 |
| MGAT1 | Chr5: 180162008-180165008 | MGAT1, Chr5: 180162008-180165008 |
| CYP4F3 | Chr19: 15611206-15614206 | CYP4F3, Chr19: 15611206-15614206 |
| C1orf104 | Chr1: 153559062-153562062 | C1orf104, Chr1: 153559062-153562062 |
| SH2B2 | Chr7: 101713665-101716665 | SH2B2, Chr7: 101713665-101716665 |
| KRTAP9-9 | Chr17: 36640777-36643777 | KRTAP9-9, Chr17: 36640777-36643777 |
| PYDC1 | Chr16: 31134396-31137396 | PYDC1, Chr16: 31134396-31137396 |
| IFITM2 | Chr11: 296606-299606 | IFITM2, Chr11: 296606-299606 |
| FOXS1 | Chr20: 29895581-29898581 | FOXS1, Chr20: 29895581-29898581 |
| ITGBL1 | Chr13: 100901466-100904466 | ITGBL1, Chr13: 100901466-100904466 |
| GALR3 | Chr22: 36547834-36550834 | GALR3, Chr22: 36547834-36550834 |
| HYAL2 | Chr3: 50333646-50336646 | HYAL2, Chr3: 50333646-50336646 |
| KRTAP9-3 | Chr17: 36640740-36643740 | KRTAP9-3, Chr17: 36640740-36643740 |
| LEFTY1 | Chr1: 224141959-224144959 | LEFTY1, Chr1: 224141959-224144959 |
| ODF3L2 | Chr19: 424483-427483 | ODF3L2, Chr19: 424483-427483 |
| CD164L2 | Chr1: 27580892-27583892 | CD164L2, Chr1: 27580892-27583892 |
| LEFTY2 | Chr1: 224194043-224197043 | LEFTY2, Chr1: 224194043-224197043 |
| KRTAP1-1 | Chr17: 36449739-36452739 | KRTAP1-1, Chr17: 36449739-36452739 |
| PCBP4 | Chr3: 51969192-51972192 | PCBP4, Chr3: 51969192-51972192 |
| C1QTNF8 | Chr16: 1084745-1087745 | C1QTNF8, Chr16: 1084745-1087745 |
| NUDT22 | Chr11: 63748813-63751813 | NUDT22, Chr11: 63748813-63751813 |
| GZMM | Chr19: 493526-496526 | GZMM, Chr19: 493526-496526 |
| HCG9 | Chr6: 30049370-30052370 | HCG9, Chr6: 30049370-30052370 |
| KRTAP4-3 | Chr17: 36576450-36579450 | KRTAP4-3, Chr17: 36576450-36579450 |
| SFN | Chr1: 27060719-27063719 | SFN, Chr1: 27060719-27063719 |
| SLC16A8 | Chr22: 36807616-36810616 | SLC16A8, Chr22: 36807616-36810616 |
| CATSPER4 | Chr1: 26388205-26391205 | CATSPER4, Chr1: 26388205-26391205 |
| LTC4S | Chr5: 179152091-179155091 | LTC4S, Chr5: 179152091-179155091 |
| BPI | Chr20: 36364465-36367465 | BPI, Chr20: 36364465-36367465 |
| F7 | Chr13: 112806605-112809605 | F7, Chr13: 112806605-112809605 |
| MMP21 | Chr10: 127452880-127455880 | MMP21, Chr10: 127452880-127455880 |
| LYPD3 | Chr19: 48660171-48663171 | LYPD3, Chr19: 48660171-48663171 |
| POU5F1 | Chr6: 31244930-31247930 | POU5F1, Chr6: 31244930-31247930 |
| IL17REL | Chr22: 48791682-48794682 | IL17REL, Chr22: 48791682-48794682 |
| PSG1 | Chr19: 48074211-48077211 | PSG1, Chr19: 48074211-48077211 |
| FAM128A | Chr2: 131965034-131968034 | FAM128A, Chr2: 131965034-131968034 |
| TMEM59L | Chr19: 18583181-18586181 | TMEM59L, Chr19: 18583181-18586181 |
| FGFRL1 | Chr4: 994109-997109 | FGFRL1, Chr4: 994109-997109 |
| FIBCD1 | Chr9: 132802558-132805558 | FIBCD1, Chr9: 132802558-132805558 |
| C20orf20 | Chr20: 60896782-60899782 | C20orf20, Chr20: 60896782-60899782 |
| FGFRL1 | Chr4: 994259-997259 | FGFRL1, Chr4: 994259-997259 |
| EGR1 | Chr5: 137827579-137830579 | EGR1, Chr5: 137827579-137830579 |
| KCTD11 | Chr17: 7194431-7197431 | KCTD11, Chr17: 7194431-7197431 |
| CBX8 | Chr17: 75383985-75386985 | CBX8, Chr17: 75383985-75386985 |
| CYHR1 | Chr8: 145660339-145663339 | CYHR1, Chr8: 145660339-145663339 |
| ATG4B | Chr2: 242224199-242227199 | ATG4B, Chr2: 242224199-242227199 |
| PANX2 | Chr22: 48949786-48952786 | PANX2, Chr22: 48949786-48952786 |
| C14orf80 | Chr14: 105027248-105030248 | C14orf80, Chr14: 105027248-105030248 |
| TRAPPC6A | Chr19: 50371825-50374825 | TRAPPC6A, Chr19: 50371825-50374825 |
| HMHA1 | Chr19: 1016673-1019673 | HMHA1, Chr19: 1016673-1019673 |
| AGBL5 | Chr2: 27126494-27129494 | AGBL5, Chr2: 27126494-27129494 |
| ICOSLG | Chr21: 44483762-44486762 | ICOSLG, Chr21: 44483762-44486762 |
| RAC3 | Chr17: 77581320-77584320 | RAC3, Chr17: 77581320-77584320 |
| CYHR1 | Chr8: 145660157-145663157 | CYHR1, Chr8: 145660157-145663157 |
| ZBTB22 | Chr6: 33391972-33394972 | ZBTB22, Chr6: 33391972-33394972 |
| COL27A1 | Chr9: 115956551-115959551 | COL27A1, Chr9: 115956551-115959551 |
| DUSP4 | Chr8: 29260741-29263741 | DUSP4, Chr8: 29260741-29263741 |
| LENG9 | Chr19: 59665206-59668206 | LENG9, Chr19: 59665206-59668206 |
| SSSCA1 | Chr11: 65093018-65096018 | SSSCA1, Chr11: 65093018-65096018 |
| FGFRL1 | Chr4: 994751-997751 | FGFRL1, Chr4: 994751-997751 |
| FAM78A | Chr9: 133140227-133143227 | FAM78A, Chr9: 133140227-133143227 |
| C10orf49 | Chr10: 13314834-13317834 | C10orf49, Chr10: 13314834-13317834 |
| RCOR2 | Chr11: 63439392-63442392 | RCOR2, Chr11: 63439392-63442392 |
| EEF1D | Chr8: 144748953-144751953 | EEF1D, Chr8: 144748953-144751953 |
| LRRC4 | Chr7: 127456738-127459738 | LRRC4, Chr7: 127456738-127459738 |
| CYHR1 | Chr8: 145659726-145662726 | CYHR1, Chr8: 145659726-145662726 |
| ZNF319 | Chr16: 56589763-56592763 | ZNF319, Chr16: 56589763-56592763 |
| OPLAH | Chr8: 145185459-145188459 | OPLAH, Chr8: 145185459-145188459 |
| HDGF2 | Chr19: 4421754-4424754 | HDGF2, Chr19: 4421754-4424754 |
| MAP3K11 | Chr11: 65136796-65139796 | MAP3K11, Chr11: 65136796-65139796 |
| THEG | Chr19: 325509-328509 | THEG, Chr19: 325509-328509 |
| ADCY3 | Chr2: 24994059-24997059 | ADCY3, Chr2: 24994059-24997059 |
| LIMD1 | Chr3: 45609826-45612826 | LIMD1, Chr3: 45609826-45612826 |
| TNFAIP8L1 | Chr19: 4589029-4592029 | TNFAIP8L1, Chr19: 4589029-4592029 |
| SLC35C1 | Chr11: 45782411-45785411 | SLC35C1, Chr11: 45782411-45785411 |
| MTP18 | Chr22: 29150219-29153219 | MTP18, Chr22: 29150219-29153219 |
| MAP3K12 | Chr12: 52178038-52181038 | MAP3K12, Chr12: 52178038-52181038 |
| RTN4RL2 | Chr11: 56983414-56986414 | RTN4RL2, Chr11: 56983414-56986414 |
| ZNF500 | Chr16: 4755667-4758667 | ZNF500, Chr16: 4755667-4758667 |
| FLII | Chr17: 18101280-18104280 | FLII, Chr17: 18101280-18104280 |
| C4orf42 | Chr4: 1232676-1235676 | C4orf42, Chr4: 1232676-1235676 |
| DTX1 | Chr12: 111978544-111981544 | DTX1, Chr12: 111978544-111981544 |
| TSTA3 | Chr8: 144768539-144771539 | TSTA3, Chr8: 144768539-144771539 |
| LHB | Chr19: 54210659-54213659 | LHB, Chr19: 54210659-54213659 |
| SNX33 | Chr15: 73726902-73729902 | SNX33, Chr15: 73726902-73729902 |
| C6orf47 | Chr6: 31735028-31738028 | C6orf47, Chr6: 31735028-31738028 |
| ARHGAP4 | Chr23: 152843392-152846392 | ARHGAP4, Chr23: 152843392-152846392 |
| XAGE3 | Chr23: 52912324-52915324 | XAGE3, Chr23: 52912324-52915324 |
| FAM89B | Chr11: 65094895-65097895 | FAM89B, Chr11: 65094895-65097895 |
| B4GALT2 | Chr1: 44217269-44220269 | B4GALT2, Chr1: 44217269-44220269 |
| TSPAN4 | Chr11: 832580-835580 | TSPAN4, Chr11: 832580-835580 |
| MAN2A2 | Chr15: 89246923-89249923 | MAN2A2, Chr15: 89246923-89249923 |
| RPL3L | Chr16: 1943180-1946180 | RPL3L, Chr16: 1943180-1946180 |
| PLA2G4F | Chr15: 40234602-40237602 | PLA2G4F, Chr15: 40234602-40237602 |
| LOC388135 | Chr15: 71829369-71832369 | LOC388135, Chr15: 71829369-71832369 |
| IL17RE | Chr3: 9917802-9920802 | IL17RE, Chr3: 9917802-9920802 |
| INHA | Chr2: 220143697-220146697 | INHA, Chr2: 220143697-220146697 |
| PDLIM2 | Chr8: 22492428-22495428 | PDLIM2, Chr8: 22492428-22495428 |
| KRTAP5-5 | Chr11: 1606108-1609108 | KRTAP5-5, Chr11: 1606108-1609108 |
| SLC16A3 | Chr17: 77781932-77784932 | SLC16A3, Chr17: 77781932-77784932 |
| AMAC1L3 | Chr17: 7323944-7326944 | AMAC1L3, Chr17: 7323944-7326944 |
| FOXH1 | Chr8: 145671026-145674026 | FOXH1, Chr8: 145671026-145674026 |
| TSSC4 | Chr11: 2378598-2381598 | TSSC4, Chr11: 2378598-2381598 |
| NFATC4 | Chr14: 23905593-23908593 | NFATC4, Chr14: 23905593-23908593 |
| IL17RE | Chr3: 9918011-9921011 | IL17RE, Chr3: 9918011-9921011 |
| TSPAN4 | Chr11: 832945-835945 | TSPAN4, Chr11: 832945-835945 |
| BEST2 | Chr19: 12722906-12725906 | BEST2, Chr19: 12722906-12725906 |
| SLC26A6 | Chr3: 48644783-48647783 | SLC26A6, Chr3: 48644783-48647783 |
| CDH24 | Chr14: 22593375-22596375 | CDH24, Chr14: 22593375-22596375 |
| PLEC1 | Chr8: 145087180-145090180 | PLEC1, Chr8: 145087180-145090180 |
| C19orf35 | Chr19: 2231675-2234675 | C19orf35, Chr19: 2231675-2234675 |
| SNAPC4 | Chr9: 138411210-138414210 | SNAPC4, Chr9: 138411210-138414210 |
| IL17RC | Chr3: 9932281-9935281 | IL17RC, Chr3: 9932281-9935281 |
| HSD17B1 | Chr17: 37956009-37959009 | HSD17B1, Chr17: 37956009-37959009 |
| WFIKKN2 | Chr17: 46266103-46269103 | WFIKKN2, Chr17: 46266103-46269103 |
| AKT1 | Chr14: 104329483-104332483 | AKT1, Chr14: 104329483-104332483 |
| RUSC1 | Chr1: 153559341-153562341 | RUSC1, Chr1: 153559341-153562341 |
| NANOS3 | Chr19: 13847449-13850449 | NANOS3, Chr19: 13847449-13850449 |
| GPR37L1 | Chr1: 200357151-200360151 | GPR37L1, Chr1: 200357151-200360151 |
| NR0B2 | Chr1: 27111547-27114547 | NR0B2, Chr1: 27111547-27114547 |
| GPIHBP1 | Chr8: 144364942-144367942 | GPIHBP1, Chr8: 144364942-144367942 |
| SLC25A22 | Chr11: 784721-787721 | SLC25A22, Chr11: 784721-787721 |
| LCN9 | Chr9: 137693488-137696488 | LCN9, Chr9: 137693488-137696488 |
| GGTLC1 | Chr20: 23914012-23917012 | GGTLC1, Chr20: 23914012-23917012 |
| SPNS3 | Chr17: 4282467-4285467 | SPNS3, Chr17: 4282467-4285467 |
| XAGE3 | Chr23: 52911557-52914557 | XAGE3, Chr23: 52911557-52914557 |
| MC1R | Chr16: 88510287-88513287 | MC1R, Chr16: 88510287-88513287 |
| TRPT1 | Chr11: 63748698-63751698 | TRPT1, Chr11: 63748698-63751698 |
| TRPT1 | Chr11: 63748757-63751757 | TRPT1, Chr11: 63748757-63751757 |
| FBN3 | Chr19: 8116885-8119885 | FBN3, Chr19: 8116885-8119885 |
| SERPINF1 | Chr17: 1610508-1613508 | SERPINF1, Chr17: 1610508-1613508 |
| C19orf41 | Chr19: 55356850-55359850 | C19orf41, Chr19: 55356850-55359850 |
| CYP4F2 | Chr19: 15868384-15871384 | CYP4F2, Chr19: 15868384-15871384 |
| C19orf21 | Chr19: 700645-703645 | C19orf21, Chr19: 700645-703645 |
| IL29 | Chr19: 44477304-44480304 | IL29, Chr19: 44477304-44480304 |
| PRCD | Chr17: 72046266-72049266 | PRCD, Chr17: 72046266-72049266 |
| 9-Sep | Chr17: 72882259-72885259 | 40795, Chr17: 72882259-72885259 |
| HSPC152 | Chr11: 63840109-63843109 | HSPC152, Chr11: 63840109-63843109 |
| SRCAP | Chr16: 30616462-30619462 | SRCAP, Chr16: 30616462-30619462 |
| LRDD | Chr11: 792964-795964 | LRDD, Chr11: 792964-795964 |
| TPSD1 | Chr16: 1244773-1247773 | TPSD1, Chr16: 1244773-1247773 |
| COL8A2 | Chr1: 36336937-36339937 | COL8A2, Chr1: 36336937-36339937 |
| RUSC1 | Chr1: 153558851-153561851 | RUSC1, Chr1: 153558851-153561851 |
| KIAA0284 | Chr14: 104401198-104404198 | KIAA0284, Chr14: 104401198-104404198 |
| SAMD1 | Chr19: 14060732-14063732 | SAMD1, Chr19: 14060732-14063732 |
| KIAA1530 | Chr4: 1329603-1332603 | KIAA1530, Chr4: 1329603-1332603 |
| FLJ10490 | Chr19: 54312709-54315709 | FLJ10490, Chr19: 54312709-54315709 |
| C3orf54 | Chr3: 49814190-49817190 | C3orf54, Chr3: 49814190-49817190 |
| LINGO1 | Chr15: 75710264-75713264 | LINGO1, Chr15: 75710264-75713264 |
| FIZ1 | Chr19: 60801205-60804205 | FIZ1, Chr19: 60801205-60804205 |
| EFEMP2 | Chr11: 65395416-65398416 | EFEMP2, Chr11: 65395416-65398416 |
| CEMP1 | Chr16: 2519920-2522920 | CEMP1, Chr16: 2519920-2522920 |
| C12orf52 | Chr12: 112106437-112109437 | C12orf52, Chr12: 112106437-112109437 |
| SLC22A2 | Chr6: 160598449-160601449 | SLC22A2, Chr6: 160598449-160601449 |
| FLJ45079 | Chr17: 73390264-73393264 | FLJ45079, Chr17: 73390264-73393264 |
| NLK | Chr17: 23392314-23395314 | NLK, Chr17: 23392314-23395314 |
| FOXJ1 | Chr17: 71646661-71649661 | FOXJ1, Chr17: 71646661-71649661 |
| SCO2 | Chr22: 49309400-49312400 | SCO2, Chr22: 49309400-49312400 |
| TUBB2C | Chr9: 139254031-139257031 | TUBB2C, Chr9: 139254031-139257031 |
| SNAPC2 | Chr19: 7889728-7892728 | SNAPC2, Chr19: 7889728-7892728 |
| C8orf58 | Chr8: 22511566-22514566 | C8orf58, Chr8: 22511566-22514566 |
| REEP6 | Chr19: 1440664-1443664 | REEP6, Chr19: 1440664-1443664 |
| LRRC45 | Chr17: 77573068-77576068 | LRRC45, Chr17: 77573068-77576068 |
| DBN1 | Chr5: 176830977-176833977 | DBN1, Chr5: 176830977-176833977 |
| RNF208 | Chr9: 139234096-139237096 | RNF208, Chr9: 139234096-139237096 |
| LNPEP | Chr5: 96295601-96298601 | LNPEP, Chr5: 96295601-96298601 |
| C11orf68 | Chr11: 65441607-65444607 | C11orf68, Chr11: 65441607-65444607 |
| LPAR2 | Chr19: 19598539-19601539 | LPAR2, Chr19: 19598539-19601539 |
| C17orf90 | Chr17: 77242523-77245523 | C17orf90, Chr17: 77242523-77245523 |
| CBFA2T3 | Chr16: 87533609-87536609 | CBFA2T3, Chr16: 87533609-87536609 |
| PAOX | Chr10: 135041230-135044230 | PAOX, Chr10: 135041230-135044230 |
| CDT1 | Chr16: 87396186-87399186 | CDT1, Chr16: 87396186-87399186 |
| ZNF446 | Chr19: 63678106-63681106 | ZNF446, Chr19: 63678106-63681106 |
| KCNJ11 | Chr11: 17365282-17368282 | KCNJ11, Chr11: 17365282-17368282 |
| C7orf51 | Chr7: 99917985-99920985 | C7orf51, Chr7: 99917985-99920985 |
| TRIM54 | Chr2: 27357603-27360603 | TRIM54, Chr2: 27357603-27360603 |
| VAV2 | Chr9: 135845726-135848726 | VAV2, Chr9: 135845726-135848726 |
| SLC9A3R1 | Chr17: 70254878-70257878 | SLC9A3R1, Chr17: 70254878-70257878 |
| NUDT8 | Chr11: 67152477-67155477 | NUDT8, Chr11: 67152477-67155477 |
| ARRDC1 | Chr9: 139618416-139621416 | ARRDC1, Chr9: 139618416-139621416 |
| ARHGDIA | Chr17: 77421027-77424027 | ARHGDIA, Chr17: 77421027-77424027 |
| CTDP1 | Chr18: 75539288-75542288 | CTDP1, Chr18: 75539288-75542288 |
| GPR172A | Chr8: 145551532-145554532 | GPR172A, Chr8: 145551532-145554532 |
| SNIP | Chr17: 34014209-34017209 | SNIP, Chr17: 34014209-34017209 |
| C1orf93 | Chr1: 2506608-2509608 | C1orf93, Chr1: 2506608-2509608 |
| TIGD3 | Chr11: 64877357-64880357 | TIGD3, Chr11: 64877357-64880357 |
| ADM2 | Chr22: 49265377-49268377 | ADM2, Chr22: 49265377-49268377 |
| POLR2L | Chr11: 831029-834029 | POLR2L, Chr11: 831029-834029 |
| LMF2 | Chr22: 49291479-49294479 | LMF2, Chr22: 49291479-49294479 |
| PRSS27 | Chr16: 2709053-2712053 | PRSS27, Chr16: 2709053-2712053 |
| PYCR1 | Chr17: 77486759-77489759 | PYCR1, Chr17: 77486759-77489759 |
| ASPHD1 | Chr16: 29818147-29821147 | ASPHD1, Chr16: 29818147-29821147 |
| C8orf55 | Chr8: 143804122-143807122 | C8orf55, Chr8: 143804122-143807122 |
| AHNAK | Chr11: 62069408-62072408 | AHNAK, Chr11: 62069408-62072408 |
| RAB11B | Chr19: 8359704-8362704 | RAB11B, Chr19: 8359704-8362704 |
| CRIP2 | Chr14: 105010675-105013675 | CRIP2, Chr14: 105010675-105013675 |
| HS3ST6 | Chr16: 1906732-1909732 | HS3ST6, Chr16: 1906732-1909732 |
| AGRN | Chr1: 943865-946865 | AGRN, Chr1: 943865-946865 |
| GLI4 | Chr8: 144419481-144422481 | GLI4, Chr8: 144419481-144422481 |
| TMEM134 | Chr11: 66991807-66994807 | TMEM134, Chr11: 66991807-66994807 |
| RHPN1 | Chr8: 144520899-144523899 | RHPN1, Chr8: 144520899-144523899 |
| EFNA2 | Chr19: 1235667-1238667 | EFNA2, Chr19: 1235667-1238667 |
| HDAC11 | Chr3: 13495323-13498323 | HDAC11, Chr3: 13495323-13498323 |
| XKR8 | Chr1: 28157590-28160590 | XKR8, Chr1: 28157590-28160590 |
| GPR137 | Chr11: 63808406-63811406 | GPR137, Chr11: 63808406-63811406 |
| C9orf23 | Chr9: 34600601-34603601 | C9orf23, Chr9: 34600601-34603601 |
| KLK15 | Chr19: 56025091-56028091 | KLK15, Chr19: 56025091-56028091 |
| EXOC3 | Chr5: 494833-497833 | EXOC3, Chr5: 494833-497833 |
| FLVCR2 | Chr14: 75113192-75116192 | FLVCR2, Chr14: 75113192-75116192 |
| DARS2 | Chr1: 172059080-172062080 | DARS2, Chr1: 172059080-172062080 |
| EFCAB4A | Chr11: 816084-819084 | EFCAB4A, Chr11: 816084-819084 |
| C19orf62 | Chr19: 17237731-17240731 | C19orf62, Chr19: 17237731-17240731 |
| PPM1M | Chr3: 52253764-52256764 | PPM1M, Chr3: 52253764-52256764 |
| KRT80 | Chr12: 50870551-50873551 | KRT80, Chr12: 50870551-50873551 |
| SSNA1 | Chr9: 139201374-139204374 | SSNA1, Chr9: 139201374-139204374 |
| NECAB2 | Chr16: 82558237-82561237 | NECAB2, Chr16: 82558237-82561237 |
| STRA13 | Chr17: 77572562-77575562 | STRA13, Chr17: 77572562-77575562 |
| LRRC14 | Chr8: 145712698-145715698 | LRRC14, Chr8: 145712698-145715698 |
| LRP3 | Chr19: 38375938-38378938 | LRP3, Chr19: 38375938-38378938 |
| C9orf75 | Chr9: 139213301-139216301 | C9orf75, Chr9: 139213301-139216301 |
| RECQL4 | Chr8: 145712508-145715508 | RECQL4, Chr8: 145712508-145715508 |
| NOXA1 | Chr9: 139436167-139439167 | NOXA1, Chr9: 139436167-139439167 |
| MTHFSD | Chr16: 85144842-85147842 | MTHFSD, Chr16: 85144842-85147842 |
| SORBS3 | Chr8: 22463695-22466695 | SORBS3, Chr8: 22463695-22466695 |
| PLXNA3 | Chr23: 153338316-153341316 | PLXNA3, Chr23: 153338316-153341316 |
| VEGFB | Chr11: 63757341-63760341 | VEGFB, Chr11: 63757341-63760341 |
| POLG | Chr15: 87677530-87680530 | POLG, Chr15: 87677530-87680530 |
| EFHD2 | Chr1: 15607527-15610527 | EFHD2, Chr1: 15607527-15610527 |
| GALNS | Chr16: 87449375-87452375 | GALNS, Chr16: 87449375-87452375 |
| POLR2I | Chr19: 41296546-41299546 | POLR2I, Chr19: 41296546-41299546 |
| LRDD | Chr11: 793745-796745 | LRDD, Chr11: 793745-796745 |
| C9orf7 | Chr9: 135313451-135316451 | C9orf7, Chr9: 135313451-135316451 |
| CASKIN1 | Chr16: 2184966-2187966 | CASKIN1, Chr16: 2184966-2187966 |
| CDC42BPG | Chr11: 64367117-64370117 | CDC42BPG, Chr11: 64367117-64370117 |
| PSCD2 | Chr19: 53662923-53665923 | PSCD2, Chr19: 53662923-53665923 |
| ABR | Chr17: 957574-960574 | ABR, Chr17: 957574-960574 |
| ACCN2 | Chr12: 48736253-48739253 | ACCN2, Chr12: 48736253-48739253 |
| KNDC1 | Chr10: 134822460-134825460 | KNDC1, Chr10: 134822460-134825460 |
| C9orf142 | Chr9: 139005190-139008190 | C9orf142, Chr9: 139005190-139008190 |
| PCBP4 | Chr3: 51975009-51978009 | PCBP4, Chr3: 51975009-51978009 |
| MAGIX | Chr23: 48905713-48908713 | MAGIX, Chr23: 48905713-48908713 |
| BID | Chr22: 16635279-16638279 | BID, Chr22: 16635279-16638279 |
| CES2 | Chr16: 65524347-65527347 | CES2, Chr16: 65524347-65527347 |
| MRPS12 | Chr19: 44111933-44114933 | MRPS12, Chr19: 44111933-44114933 |
| GTPBP3 | Chr19: 17307855-17310855 | GTPBP3, Chr19: 17307855-17310855 |
| GTF2IRD1 | Chr7: 73504872-73507872 | GTF2IRD1, Chr7: 73504872-73507872 |
| SYNGR4 | Chr19: 53557968-53560968 | SYNGR4, Chr19: 53557968-53560968 |
| ARHGEF4 | Chr2: 131389193-131392193 | ARHGEF4, Chr2: 131389193-131392193 |
| SERTAD4 | Chr1: 208471317-208474317 | SERTAD4, Chr1: 208471317-208474317 |
| DERL3 | Chr22: 22509701-22512701 | DERL3, Chr22: 22509701-22512701 |
| TMED1 | Chr19: 10806483-10809483 | TMED1, Chr19: 10806483-10809483 |
| TBC1D9B | Chr5: 179265962-179268962 | TBC1D9B, Chr5: 179265962-179268962 |
| DLGAP4 | Chr20: 34356216-34359216 | DLGAP4, Chr20: 34356216-34359216 |
| ROGDI | Chr16: 4791175-4794175 | ROGDI, Chr16: 4791175-4794175 |
| SNED1 | Chr2: 241585427-241588427 | SNED1, Chr2: 241585427-241588427 |
| RNPEPL1 | Chr2: 241155276-241158276 | RNPEPL1, Chr2: 241155276-241158276 |
| EEPD1 | Chr7: 36157860-36160860 | EEPD1, Chr7: 36157860-36160860 |
| PKN1 | Chr19: 14403665-14406665 | PKN1, Chr19: 14403665-14406665 |
| ALS2CL | Chr3: 46708675-46711675 | ALS2CL, Chr3: 46708675-46711675 |
| BCL7C | Chr16: 30811400-30814400 | BCL7C, Chr16: 30811400-30814400 |
| BRD9 | Chr5: 944415-947415 | BRD9, Chr5: 944415-947415 |
| CAPN5 | Chr11: 76454139-76457139 | CAPN5, Chr11: 76454139-76457139 |
| KIAA1787 | Chr17: 7171862-7174862 | KIAA1787, Chr17: 7171862-7174862 |
| FICD | Chr12: 107431680-107434680 | FICD, Chr12: 107431680-107434680 |
| DOC2A | Chr16: 29928402-29931402 | DOC2A, Chr16: 29928402-29931402 |
| MAP1LC3A | Chr20: 32608679-32611679 | MAP1LC3A, Chr20: 32608679-32611679 |
| SLC46A1 | Chr17: 23755855-23758855 | SLC46A1, Chr17: 23755855-23758855 |
| C14orf172 | Chr14: 103063761-103066761 | C14orf172, Chr14: 103063761-103066761 |
| KCNT1 | Chr9: 137732358-137735358 | KCNT1, Chr9: 137732358-137735358 |
| GPR3 | Chr1: 27590238-27593238 | GPR3, Chr1: 27590238-27593238 |
| PITX3 | Chr10: 103989721-103992721 | PITX3, Chr10: 103989721-103992721 |
| TBX6 | Chr16: 30009209-30012209 | TBX6, Chr16: 30009209-30012209 |
| SRC | Chr20: 35406470-35409470 | SRC, Chr20: 35406470-35409470 |
| F2RL1 | Chr5: 76149109-76152109 | F2RL1, Chr5: 76149109-76152109 |
| CDH15 | Chr16: 87764163-87767163 | CDH15, Chr16: 87764163-87767163 |
| APOA4 | Chr11: 116197721-116200721 | APOA4, Chr11: 116197721-116200721 |
| NTAN1 | Chr16: 15055834-15058834 | NTAN1, Chr16: 15055834-15058834 |
| GPAA1 | Chr8: 145208011-145211011 | GPAA1, Chr8: 145208011-145211011 |
| RNASET2 | Chr6: 167288567-167291567 | RNASET2, Chr6: 167288567-167291567 |
| LRRC3 | Chr21: 44698320-44701320 | LRRC3, Chr21: 44698320-44701320 |
| GABRD | Chr1: 1939127-1942127 | GABRD, Chr1: 1939127-1942127 |
| CPSF3L | Chr1: 1248409-1251409 | CPSF3L, Chr1: 1248409-1251409 |
| SH2B3 | Chr12: 110326634-110329634 | SH2B3, Chr12: 110326634-110329634 |
| RAD9A | Chr11: 66914498-66917498 | RAD9A, Chr11: 66914498-66917498 |
| LOC652968 | Chr22: 29014116-29017116 | LOC652968, Chr22: 29014116-29017116 |
| EXOSC4 | Chr8: 145204009-145207009 | EXOSC4, Chr8: 145204009-145207009 |
| SCYL1 | Chr11: 65047623-65050623 | SCYL1, Chr11: 65047623-65050623 |
| SLC39A13 | Chr11: 47385126-47388126 | SLC39A13, Chr11: 47385126-47388126 |
| FAM50A | Chr23: 153324201-153327201 | FAM50A, Chr23: 153324201-153327201 |
| MRPS12 | Chr19: 44111687-44114687 | MRPS12, Chr19: 44111687-44114687 |
| ZC3H12A | Chr1: 37711205-37714205 | ZC3H12A, Chr1: 37711205-37714205 |
| SNAI3 | Chr16: 87278883-87281883 | SNAI3, Chr16: 87278883-87281883 |
| SCRN1 | Chr7: 29994402-29997402 | SCRN1, Chr7: 29994402-29997402 |
| YPEL3 | Chr16: 30013522-30016522 | YPEL3, Chr16: 30013522-30016522 |
| ARL4D | Chr17: 38830378-38833378 | ARL4D, Chr17: 38830378-38833378 |
| RNF26 | Chr11: 118708946-118711946 | RNF26, Chr11: 118708946-118711946 |
| SLC35E4 | Chr22: 29360292-29363292 | SLC35E4, Chr22: 29360292-29363292 |
| NIPSNAP1 | Chr22: 28305644-28308644 | NIPSNAP1, Chr22: 28305644-28308644 |
| TBXA2R | Chr19: 3556158-3559158 | TBXA2R, Chr19: 3556158-3559158 |
| LY6H | Chr8: 144311616-144314616 | LY6H, Chr8: 144311616-144314616 |
| PSRC1 | Chr1: 109625794-109628794 | PSRC1, Chr1: 109625794-109628794 |
| SMPD1 | Chr11: 6366730-6369730 | SMPD1, Chr11: 6366730-6369730 |
| FNDC5 | Chr1: 33107434-33110434 | FNDC5, Chr1: 33107434-33110434 |
| ABCA7 | Chr19: 989601-992601 | ABCA7, Chr19: 989601-992601 |
| KLHDC8B | Chr3: 49182571-49185571 | KLHDC8B, Chr3: 49182571-49185571 |
| SH3BP1 | Chr22: 36364129-36367129 | SH3BP1, Chr22: 36364129-36367129 |
| ELOVL1 | Chr1: 43604786-43607786 | ELOVL1, Chr1: 43604786-43607786 |
| WDR38 | Chr9: 126654075-126657075 | WDR38, Chr9: 126654075-126657075 |
| TUFM | Chr16: 28763730-28766730 | TUFM, Chr16: 28763730-28766730 |
| SPOCD1 | Chr1: 32052667-32055667 | SPOCD1, Chr1: 32052667-32055667 |
| DBN1 | Chr5: 176831800-176834800 | DBN1, Chr5: 176831800-176834800 |
| BOK | Chr2: 242145364-242148364 | BOK, Chr2: 242145364-242148364 |
| SUSD1 | Chr9: 113975877-113978877 | SUSD1, Chr9: 113975877-113978877 |
| EDN3 | Chr20: 57307393-57310393 | EDN3, Chr20: 57307393-57310393 |
| FOXK1 | Chr7: 4686955-4689955 | FOXK1, Chr7: 4686955-4689955 |
| GPR139 | Chr16: 19991101-19994101 | GPR139, Chr16: 19991101-19994101 |
| KIF3C | Chr2: 26057447-26060447 | KIF3C, Chr2: 26057447-26060447 |
| PFKL | Chr21: 44542857-44545857 | PFKL, Chr21: 44542857-44545857 |
| TYRO3 | Chr15: 39637023-39640023 | TYRO3, Chr15: 39637023-39640023 |
| JMJD4 | Chr1: 225988235-225991235 | JMJD4, Chr1: 225988235-225991235 |
| HRH3 | Chr20: 60227218-60230218 | HRH3, Chr20: 60227218-60230218 |
| IRAK1 | Chr23: 152937036-152940036 | IRAK1, Chr23: 152937036-152940036 |
| HPS6 | Chr10: 103813636-103816636 | HPS6, Chr10: 103813636-103816636 |
| C10orf93 | Chr10: 134604554-134607554 | C10orf93, Chr10: 134604554-134607554 |
| SLC4A3 | Chr2: 220199035-220202035 | SLC4A3, Chr2: 220199035-220202035 |
| RASGEF1C | Chr5: 179567236-179570236 | RASGEF1C, Chr5: 179567236-179570236 |
| MXD3 | Chr5: 176669987-176672987 | MXD3, Chr5: 176669987-176672987 |
| GALNT12 | Chr9: 100608301-100611301 | GALNT12, Chr9: 100608301-100611301 |
| PHKG2 | Chr16: 30665737-30668737 | PHKG2, Chr16: 30665737-30668737 |
| USP2 | Chr11: 118738602-118741602 | USP2, Chr11: 118738602-118741602 |
| ZNF581 | Chr19: 60845297-60848297 | ZNF581, Chr19: 60845297-60848297 |
| IDUA | Chr4: 969284-972284 | IDUA, Chr4: 969284-972284 |
| TRPV4 | Chr12: 108754095-108757095 | TRPV4, Chr12: 108754095-108757095 |
| GTF2IRD1 | Chr7: 73504555-73507555 | GTF2IRD1, Chr7: 73504555-73507555 |
| SGSM1 | Chr22: 23530635-23533635 | SGSM1, Chr22: 23530635-23533635 |
| FXYD5 | Chr19: 40335966-40338966 | FXYD5, Chr19: 40335966-40338966 |
| TTYH1 | Chr19: 59616946-59619946 | TTYH1, Chr19: 59616946-59619946 |
| MRPL55 | Chr1: 226362136-226365136 | MRPL55, Chr1: 226362136-226365136 |
| SLC22A18 | Chr11: 2878599-2881599 | SLC22A18, Chr11: 2878599-2881599 |
| CORO1B | Chr11: 66966059-66969059 | CORO1B, Chr11: 66966059-66969059 |
| DHDH | Chr19: 54127250-54130250 | DHDH, Chr19: 54127250-54130250 |
| KDELR3 | Chr22: 37192528-37195528 | KDELR3, Chr22: 37192528-37195528 |
| PRKCD | Chr3: 53168762-53171762 | PRKCD, Chr3: 53168762-53171762 |
| GIPC3 | Chr19: 3535068-3538068 | GIPC3, Chr19: 3535068-3538068 |
| SLC12A7 | Chr5: 1163672-1166672 | SLC12A7, Chr5: 1163672-1166672 |
| SYCE1 | Chr10: 135231366-135234366 | SYCE1, Chr10: 135231366-135234366 |
| SYT2 | Chr1: 200944668-200947668 | SYT2, Chr1: 200944668-200947668 |
| DCXR | Chr17: 77587362-77590362 | DCXR, Chr17: 77587362-77590362 |
| KAT5 | Chr11: 65234564-65237564 | KAT5, Chr11: 65234564-65237564 |
| BCKDK | Chr16: 31025662-31028662 | BCKDK, Chr16: 31025662-31028662 |
| BTBD2 | Chr19: 1965202-1968202 | BTBD2, Chr19: 1965202-1968202 |
| PCP2 | Chr19: 7603070-7606070 | PCP2, Chr19: 7603070-7606070 |
| OTUB2 | Chr14: 93560976-93563976 | OTUB2, Chr14: 93560976-93563976 |
| TNIP2 | Chr4: 2726359-2729359 | TNIP2, Chr4: 2726359-2729359 |
| ABCG4 | Chr11: 118523459-118526459 | ABCG4, Chr11: 118523459-118526459 |
| GPR84 | Chr12: 53043025-53046025 | GPR84, Chr12: 53043025-53046025 |
| RAB26 | Chr16: 2137151-2140151 | RAB26, Chr16: 2137151-2140151 |
| TOR2A | Chr9: 129535925-129538925 | TOR2A, Chr9: 129535925-129538925 |
| DDX27 | Chr20: 47267790-47270790 | DDX27, Chr20: 47267790-47270790 |
| GPR39 | Chr2: 132889116-132892116 | GPR39, Chr2: 132889116-132892116 |
| GRIK2 | Chr6: 101952125-101955125 | GRIK2, Chr6: 101952125-101955125 |
| MAN2B2 | Chr4: 6626302-6629302 | MAN2B2, Chr4: 6626302-6629302 |
| C1QL4 | Chr12: 48015738-48018738 | C1QL4, Chr12: 48015738-48018738 |
| LYPLA2 | Chr1: 23988732-23991732 | LYPLA2, Chr1: 23988732-23991732 |
| C9orf140 | Chr9: 139083349-139086349 | C9orf140, Chr9: 139083349-139086349 |
| DHRS13 | Chr17: 24252715-24255715 | DHRS13, Chr17: 24252715-24255715 |
| PRRX2 | Chr9: 131466240-131469240 | PRRX2, Chr9: 131466240-131469240 |
| SLC41A3 | Chr3: 127284324-127287324 | SLC41A3, Chr3: 127284324-127287324 |
| PIGO | Chr9: 35085046-35088046 | PIGO, Chr9: 35085046-35088046 |
| PIGO | Chr9: 35085079-35088079 | PIGO, Chr9: 35085079-35088079 |
| SLC12A8 | Chr3: 126412799-126415799 | SLC12A8, Chr3: 126412799-126415799 |
| F11R | Chr1: 159256257-159259257 | F11R, Chr1: 159256257-159259257 |
| P2RX5 | Chr17: 3544832-3547832 | P2RX5, Chr17: 3544832-3547832 |
| ARSJ | Chr4: 115118827-115121827 | ARSJ, Chr4: 115118827-115121827 |
| PROK1 | Chr1: 110793810-110796810 | PROK1, Chr1: 110793810-110796810 |
| MUC5B | Chr11: 1199371-1202371 | MUC5B, Chr11: 1199371-1202371 |
| CYBA | Chr16: 87243458-87246458 | CYBA, Chr16: 87243458-87246458 |
| LSP1 | Chr11: 1829275-1832275 | LSP1, Chr11: 1829275-1832275 |
| CBFA2T3 | Chr16: 87569402-87572402 | CBFA2T3, Chr16: 87569402-87572402 |
| CENTB5 | Chr1: 1227003-1230003 | CENTB5, Chr1: 1227003-1230003 |
| TNFRSF14 | Chr1: 2485113-2488113 | TNFRSF14, Chr1: 2485113-2488113 |
| GJC2 | Chr1: 226402675-226405675 | GJC2, Chr1: 226402675-226405675 |
| ESPNL | Chr2: 238672189-238675189 | ESPNL, Chr2: 238672189-238675189 |
| C2orf54 | Chr2: 241478625-241481625 | C2orf54, Chr2: 241478625-241481625 |
| C2orf54 | Chr2: 241482746-241485746 | C2orf54, Chr2: 241482746-241485746 |
| GAL3ST2 | Chr2: 242363412-242366412 | GAL3ST2, Chr2: 242363412-242366412 |
| PDCD1 | Chr2: 242448231-242451231 | PDCD1, Chr2: 242448231-242451231 |
| SLC6A18 | Chr5: 1276969-1279969 | SLC6A18, Chr5: 1276969-1279969 |
| GPR146 | Chr7: 1062166-1065166 | GPR146, Chr7: 1062166-1065166 |
| AQP1 | Chr7: 30916492-30919492 | AQP1, Chr7: 30916492-30919492 |
| KCNH2 | Chr7: 150282348-150285348 | KCNH2, Chr7: 150282348-150285348 |
| EPB49 | Chr8: 21969821-21972821 | EPB49, Chr8: 21969821-21972821 |
| LYNX1 | Chr8: 143846748-143849748 | LYNX1, Chr8: 143846748-143849748 |
| LY6D | Chr8: 143863510-143866510 | LY6D, Chr8: 143863510-143866510 |
| PLEC1 | Chr8: 145084246-145087246 | PLEC1, Chr8: 145084246-145087246 |
| PARP10 | Chr8: 145131123-145134123 | PARP10, Chr8: 145131123-145134123 |
| SLC39A4 | Chr8: 145611225-145614225 | SLC39A4, Chr8: 145611225-145614225 |
| SLC39A4 | Chr8: 145611581-145614581 | SLC39A4, Chr8: 145611581-145614581 |
| SOHLH1 | Chr9: 137729695-137732695 | SOHLH1, Chr9: 137729695-137732695 |
| GPSM1 | Chr9: 138347225-138350225 | GPSM1, Chr9: 138347225-138350225 |
| EGFL7 | Chr9: 138675697-138678697 | EGFL7, Chr9: 138675697-138678697 |
| EGFL7 | Chr9: 138675702-138678702 | EGFL7, Chr9: 138675702-138678702 |
| LCN8 | Chr9: 138771052-138774052 | LCN8, Chr9: 138771052-138774052 |
| MAMDC4 | Chr9: 138865139-138868139 | MAMDC4, Chr9: 138865139-138868139 |
| FLJ45224 | Chr9: 138995765-138998765 | FLJ45224, Chr9: 138995765-138998765 |
| SLC34A3 | Chr9: 139243705-139246705 | SLC34A3, Chr9: 139243705-139246705 |
| MUC6 | Chr11: 1025206-1028206 | MUC6, Chr11: 1025206-1028206 |
| SYT8 | Chr11: 1810749-1813749 | SYT8, Chr11: 1810749-1813749 |
| TSPAN32 | Chr11: 2278318-2281318 | TSPAN32, Chr11: 2278318-2281318 |
| TRPM5 | Chr11: 2399351-2402351 | TRPM5, Chr11: 2399351-2402351 |
| CYFIP1 | Chr15: 20505695-20508695 | CYFIP1, Chr15: 20505695-20508695 |
| MPFL | Chr16: 771427-774427 | MPFL, Chr16: 771427-774427 |
| IGFALS | Chr16: 1782210-1785210 | IGFALS, Chr16: 1782210-1785210 |
| ZNF469 | Chr16: 87019879-87022879 | ZNF469, Chr16: 87019879-87022879 |
| LOC390748 | Chr16: 87459015-87462015 | LOC390748, Chr16: 87459015-87462015 |
| FSCN2 | Chr17: 77108511-77111511 | FSCN2, Chr17: 77108511-77111511 |
| HPN | Chr19: 40221749-40224749 | HPN, Chr19: 40221749-40224749 |
| TNFRSF6B | Chr20: 61795231-61798231 | TNFRSF6B, Chr20: 61795231-61798231 |
| LIME1 | Chr20: 61836921-61839921 | LIME1, Chr20: 61836921-61839921 |
| AIRE | Chr21: 44533075-44536075 | AIRE, Chr21: 44533075-44536075 |
| AVPR2 | Chr23: 152822121-152825121 | AVPR2, Chr23: 152822121-152825121 |
| PLXNB3 | Chr23: 152681404-152684404 | PLXNB3, Chr23: 152681404-152684404 |
| OBSCN | Chr1: 226460983-226463983 | OBSCN, Chr1: 226460983-226463983 |
| ZAP70 | Chr2: 97715800-97718800 | ZAP70, Chr2: 97715800-97718800 |
| GPR35 | Chr2: 241215967-241218967 | GPR35, Chr2: 241215967-241218967 |
| PTP4A3 | Chr8: 142499688-142502688 | PTP4A3, Chr8: 142499688-142502688 |
| CARD9 | Chr9: 138386439-138389439 | CARD9, Chr9: 138386439-138389439 |
| MUC2 | Chr11: 1063374-1066374 | MUC2, Chr11: 1063374-1066374 |
| F10 | Chr13: 112823613-112826613 | F10, Chr13: 112823613-112826613 |
| LOC124220 | Chr16: 2818673-2821673 | LOC124220, Chr16: 2818673-2821673 |
| SBNO2 | Chr19: 1081773-1084773 | SBNO2, Chr19: 1081773-1084773 |
| TMEM86B | Chr19: 60430944-60433944 | TMEM86B, Chr19: 60430944-60433944 |
| BIRC7 | Chr20: 61336220-61339220 | BIRC7, Chr20: 61336220-61339220 |
| COL20A1 | Chr20: 61393482-61396482 | COL20A1, Chr20: 61393482-61396482 |
| TCEA2 | Chr20: 62157382-62160382 | TCEA2, Chr20: 62157382-62160382 |
| KRTAP10-4 | Chr21: 44816533-44819533 | KRTAP10-4, Chr21: 44816533-44819533 |
| C21orf123 | Chr21: 45667913-45670913 | C21orf123, Chr21: 45667913-45670913 |
| OSM | Chr22: 28991340-28994340 | OSM, Chr22: 28991340-28994340 |
| NEU4 | Chr2: 242399245-242402245 | NEU4, Chr2: 242399245-242402245 |
| SLC25A34 | Chr1: 15933895-15936895 | SLC25A34, Chr1: 15933895-15936895 |
| MYL5 | Chr4: 660210-663210 | MYL5, Chr4: 660210-663210 |
| DBH | Chr9: 135489805-135492805 | DBH, Chr9: 135489805-135492805 |
| LCN10 | Chr9: 138755732-138758732 | LCN10, Chr9: 138755732-138758732 |
| LCN12 | Chr9: 138965088-138968088 | LCN12, Chr9: 138965088-138968088 |
| TNNT3 | Chr11: 1895874-1898874 | TNNT3, Chr11: 1895874-1898874 |
| KCNQ1 | Chr11: 2437759-2440759 | KCNQ1, Chr11: 2437759-2440759 |
| CABP2 | Chr11: 67045975-67048975 | CABP2, Chr11: 67045975-67048975 |
| TBX10 | Chr11: 67162107-67165107 | TBX10, Chr11: 67162107-67165107 |
| TCIRG1 | Chr11: 67565522-67568522 | TCIRG1, Chr11: 67565522-67568522 |
| GRK1 | Chr13: 113368097-113371097 | GRK1, Chr13: 113368097-113371097 |
| PDIA2 | Chr16: 271618-274618 | PDIA2, Chr16: 271618-274618 |
| HSD3B7 | Chr16: 30902532-30905532 | HSD3B7, Chr16: 30902532-30905532 |
| LCAT | Chr16: 66534016-66537016 | LCAT, Chr16: 66534016-66537016 |
| C18orf1 | Chr18: 13609366-13612366 | C18orf1, Chr18: 13609366-13612366 |
| TNNI2 | Chr11: 1815978-1818978 | TNNI2, Chr11: 1815978-1818978 |
| HSPB7 | Chr1: 16216372-16219372 | HSPB7, Chr1: 16216372-16219372 |
| CLCNKB | Chr1: 16241333-16244333 | CLCNKB, Chr1: 16241333-16244333 |
| LYPD2 | Chr8: 143829454-143832454 | LYPD2, Chr8: 143829454-143832454 |
| SYT15 | Chr10: 46389107-46392107 | SYT15, Chr10: 46389107-46392107 |
| INS-IGF2 | Chr11: 2137515-2140515 | INS-IGF2, Chr11: 2137515-2140515 |
| RIN1 | Chr11: 65859076-65862076 | RIN1, Chr11: 65859076-65862076 |
| PTPRCAP | Chr11: 66960229-66963229 | PTPRCAP, Chr11: 66960229-66963229 |
| P2RY11 | Chr19: 10081696-10084696 | P2RY11, Chr19: 10081696-10084696 |
| COL18A1 | Chr21: 45698351-45701351 | COL18A1, Chr21: 45698351-45701351 |
| CRB2 | Chr9: 125156768-125159768 | CRB2, Chr9: 125156768-125159768 |
| ADCY8 | Chr8: 132120517-132123517 | ADCY8, Chr8: 132120517-132123517 |
| TMEM184A | Chr7: 1561092-1564092 | TMEM184A, Chr7: 1561092-1564092 |
| SSPO | Chr7: 149102563-149105563 | SSPO, Chr7: 149102563-149105563 |
| EPB49 | Chr8: 21971131-21974131 | EPB49, Chr8: 21971131-21974131 |
| FLJ43860 | Chr8: 142585012-142588012 | FLJ43860, Chr8: 142585012-142588012 |
| LSP1 | Chr11: 1844978-1847978 | LSP1, Chr11: 1844978-1847978 |
| TH | Chr11: 2148111-2151111 | TH, Chr11: 2148111-2151111 |
| GALNT9 | Chr12: 131255026-131258026 | GALNT9, Chr12: 131255026-131258026 |
| C14orf180 | Chr14: 104115600-104118600 | C14orf180, Chr14: 104115600-104118600 |
| FES | Chr15: 89227212-89230212 | FES, Chr15: 89227212-89230212 |
| ITGB4 | Chr17: 71230870-71233870 | ITGB4, Chr17: 71230870-71233870 |
| UNC13D | Chr17: 71350893-71353893 | UNC13D, Chr17: 71350893-71353893 |
| C20orf151 | Chr20: 60434484-60437484 | C20orf151, Chr20: 60434484-60437484 |
| TSSK2 | Chr22: 17497289-17500289 | TSSK2, Chr22: 17497289-17500289 |
| BAIAP2L2 | Chr22: 36835122-36838122 | BAIAP2L2, Chr22: 36835122-36838122 |
| RENBP | Chr23: 152861926-152864926 | RENBP, Chr23: 152861926-152864926 |
| GPR17 | Chr2: 128118716-128121716 | GPR17, Chr2: 128118716-128121716 |
| UCN2 | Chr3: 48574705-48577705 | UCN2, Chr3: 48574705-48577705 |
| GPR132 | Chr14: 104601299-104604299 | GPR132, Chr14: 104601299-104604299 |
| RHBDL1 | Chr16: 664575-667575 | RHBDL1, Chr16: 664575-667575 |
| CD300C | Chr17: 70052377-70055377 | CD300C, Chr17: 70052377-70055377 |
| FLJ44968 | Chr19: 19499219-19502219 | FLJ44968, Chr19: 19499219-19502219 |
| AIRE | Chr21: 44528690-44531690 | AIRE, Chr21: 44528690-44531690 |
| ACCN4 | Chr2: 220085795-220088795 | ACCN4, Chr2: 220085795-220088795 |
| CPZ | Chr4: 8643834-8646834 | CPZ, Chr4: 8643834-8646834 |
| FRMD1 | Chr6: 168221188-168224188 | FRMD1, Chr6: 168221188-168224188 |
| CHRM4 | Chr11: 46363183-46366183 | CHRM4, Chr11: 46363183-46366183 |
| COMT | Chr22: 18328569-18331569 | COMT, Chr22: 18328569-18331569 |
| MMEL1 | Chr1: 2552789-2555789 | MMEL1, Chr1: 2552789-2555789 |
| TGM1 | Chr14: 23800756-23803756 | TGM1, Chr14: 23800756-23803756 |
| MYO15A | Chr17: 17951244-17954244 | MYO15A, Chr17: 17951244-17954244 |
| KRT16 | Chr17: 37021105-37024105 | KRT16, Chr17: 37021105-37024105 |
| RBMY1F | Chr24: 22862897-22865897 | RBMY1F, Chr24: 22862897-22865897 |
| TNFRSF4 | Chr1: 1137875-1140875 | TNFRSF4, Chr1: 1137875-1140875 |
| ALPI | Chr2: 233027576-233030576 | ALPI, Chr2: 233027576-233030576 |
| FUT7 | Chr9: 139045613-139048613 | FUT7, Chr9: 139045613-139048613 |
| CEND1 | Chr11: 778626-781626 | CEND1, Chr11: 778626-781626 |
| RPS6KA4 | Chr11: 63881700-63884700 | RPS6KA4, Chr11: 63881700-63884700 |
| GPR97 | Chr16: 56258171-56261171 | GPR97, Chr16: 56258171-56261171 |
| ALDH3A1 | Chr17: 19590700-19593700 | ALDH3A1, Chr17: 19590700-19593700 |
| LOC55908 | Chr19: 11208316-11211316 | LOC55908, Chr19: 11208316-11211316 |
| FFAR1 | Chr19: 40532794-40535794 | FFAR1, Chr19: 40532794-40535794 |
| ADORA2A | Chr22: 23152029-23155029 | ADORA2A, Chr22: 23152029-23155029 |
| KNCN | Chr1: 46787974-46790974 | KNCN, Chr1: 46787974-46790974 |
| FAM92B | Chr16: 83702115-83705115 | FAM92B, Chr16: 83702115-83705115 |
| AMPD2 | Chr1: 109968071-109971071 | AMPD2, Chr1: 109968071-109971071 |
| PTPN7 | Chr1: 200394242-200397242 | PTPN7, Chr1: 200394242-200397242 |
| INPP5D | Chr2: 233631779-233634779 | INPP5D, Chr2: 233631779-233634779 |
| FLJ33590 | Chr2: 242459058-242462058 | FLJ33590, Chr2: 242459058-242462058 |
| STAB1 | Chr3: 52502895-52505895 | STAB1, Chr3: 52502895-52505895 |
| PSAPL1 | Chr4: 7486100-7489100 | PSAPL1, Chr4: 7486100-7489100 |
| HR | Chr8: 22042475-22045475 | HR, Chr8: 22042475-22045475 |
| RBP3 | Chr10: 48009497-48012497 | RBP3, Chr10: 48009497-48012497 |
| ZMIZ1 | Chr10: 80590375-80593375 | ZMIZ1, Chr10: 80590375-80593375 |
| FLJ36208 | Chr16: 555532-558532 | FLJ36208, Chr16: 555532-558532 |
| RHOT2 | Chr16: 656633-659633 | RHOT2, Chr16: 656633-659633 |
| TMEM105 | Chr17: 76917569-76920569 | TMEM105, Chr17: 76917569-76920569 |
| HOXD4 | Chr2: 176722858-176725858 | HOXD4, Chr2: 176722858-176725858 |
| GPR55 | Chr2: 231496685-231499685 | GPR55, Chr2: 231496685-231499685 |
| PRLH | Chr2: 238138455-238141455 | PRLH, Chr2: 238138455-238141455 |
| SH3TC1 | Chr4: 8250459-8253459 | SH3TC1, Chr4: 8250459-8253459 |
| FOXI1 | Chr5: 169463994-169466994 | FOXI1, Chr5: 169463994-169466994 |
| PSCA | Chr8: 143757376-143760376 | PSCA, Chr8: 143757376-143760376 |
| GPRIN2 | Chr10: 46412051-46415051 | GPRIN2, Chr10: 46412051-46415051 |
| SIPA1 | Chr11: 65162667-65165667 | SIPA1, Chr11: 65162667-65165667 |
| GPR152 | Chr11: 66975276-66978276 | GPR152, Chr11: 66975276-66978276 |
| GNG13 | Chr16: 789234-792234 | GNG13, Chr16: 789234-792234 |
| DBNDD1 | Chr16: 88602530-88605530 | DBNDD1, Chr16: 88602530-88605530 |
| TMEM88 | Chr17: 7697608-7700608 | TMEM88, Chr17: 7697608-7700608 |
| TCAP | Chr17: 35073624-35076624 | TCAP, Chr17: 35073624-35076624 |
| TMEM16G | Chr2: 241775096-241778096 | TMEM16G, Chr2: 241775096-241778096 |
| VGLL4 | Chr3: 11583898-11586898 | VGLL4, Chr3: 11583898-11586898 |
| C9orf117 | Chr9: 129507591-129510591 | C9orf117, Chr9: 129507591-129510591 |
| SLC22A11 | Chr11: 64078173-64081173 | SLC22A11, Chr11: 64078173-64081173 |
| C14orf73 | Chr14: 102634733-102637733 | C14orf73, Chr14: 102634733-102637733 |
| WFIKKN1 | Chr16: 619512-622512 | WFIKKN1, Chr16: 619512-622512 |
| FAM173A | Chr16: 709658-712658 | FAM173A, Chr16: 709658-712658 |
| CLCNKA | Chr1: 16219572-16222572 | CLCNKA, Chr1: 16219572-16222572 |
| CLDN15 | Chr7: 100666336-100669336 | CLDN15, Chr7: 100666336-100669336 |
| SARDH | Chr9: 135593363-135596363 | SARDH, Chr9: 135593363-135596363 |
| CXCL12 | Chr10: 44199048-44202048 | CXCL12, Chr10: 44199048-44202048 |
| TMEM82 | Chr1: 15940003-15943003 | TMEM82, Chr1: 15940003-15943003 |
| C10orf91 | Chr10: 134107203-134110203 | C10orf91, Chr10: 134107203-134110203 |
| SLC22A20 | Chr11: 64736384-64739384 | SLC22A20, Chr11: 64736384-64739384 |
| SIPA1 | Chr11: 65160670-65163670 | SIPA1, Chr11: 65160670-65163670 |
| WDR90 | Chr16: 637863-640863 | WDR90, Chr16: 637863-640863 |
| NLRC3 | Chr16: 3565893-3568893 | NLRC3, Chr16: 3565893-3568893 |
| C16orf3 | Chr16: 88622310-88625310 | C16orf3, Chr16: 88622310-88625310 |
| LIM2 | Chr19: 56581509-56584509 | LIM2, Chr19: 56581509-56584509 |
| FER1L5 | Chr2: 96723420-96726420 | FER1L5, Chr2: 96723420-96726420 |
| KRT17 | Chr17: 37032835-37035835 | KRT17, Chr17: 37032835-37035835 |
| CD79B | Chr17: 59361936-59364936 | CD79B, Chr17: 59361936-59364936 |
| LOC553158 | Chr22: 43487632-43490632 | LOC553158, Chr22: 43487632-43490632 |
| CGB8 | Chr19: 54242680-54245680 | CGB8, Chr19: 54242680-54245680 |
| ZDHHC11 | Chr5: 902601-905601 | ZDHHC11, Chr5: 902601-905601 |
| C10orf10 | Chr10: 44792836-44795836 | C10orf10, Chr10: 44792836-44795836 |
| LOC339123 | Chr16: 672940-675940 | LOC339123, Chr16: 672940-675940 |
| 9-Sep | Chr17: 72794067-72797067 | 40795, Chr17: 72794067-72797067 |
| OTOF | Chr2: 26552914-26555914 | OTOF, Chr2: 26552914-26555914 |
| SEMA3G | Chr3: 52452583-52455583 | SEMA3G, Chr3: 52452583-52455583 |
| RGS12 | Chr4: 3340021-3343021 | RGS12, Chr4: 3340021-3343021 |
| PPP2R2C | Chr4: 6432998-6435998 | PPP2R2C, Chr4: 6432998-6435998 |
| C8orf73 | Chr8: 144724571-144727571 | C8orf73, Chr8: 144724571-144727571 |
| LOC146325 | Chr16: 548922-551922 | LOC146325, Chr16: 548922-551922 |
| METRN | Chr16: 703673-706673 | METRN, Chr16: 703673-706673 |
| RPUSD1 | Chr16: 776884-779884 | RPUSD1, Chr16: 776884-779884 |
| CHTF18 | Chr16: 777122-780122 | CHTF18, Chr16: 777122-780122 |
| PRDM9 | Chr5: 23541980-23544980 | PRDM9, Chr5: 23541980-23544980 |
| C19orf46 | Chr19: 41190012-41193012 | C19orf46, Chr19: 41190012-41193012 |
| LDB3 | Chr10: 88416800-88419800 | LDB3, Chr10: 88416800-88419800 |
| GPR148 | Chr2: 131201692-131204692 | GPR148, Chr2: 131201692-131204692 |
| PTPRE | Chr10: 129734323-129737323 | PTPRE, Chr10: 129734323-129737323 |
| FXYD2 | Chr11: 117202517-117205517 | FXYD2, Chr11: 117202517-117205517 |
| LOC388199 | Chr16: 793943-796943 | LOC388199, Chr16: 793943-796943 |
| POL3S | Chr16: 31006131-31009131 | POL3S, Chr16: 31006131-31009131 |
| SCN4A | Chr17: 59402510-59405510 | SCN4A, Chr17: 59402510-59405510 |
| C20orf186 | Chr20: 31132915-31135915 | C20orf186, Chr20: 31132915-31135915 |
| LY6E | Chr8: 144169776-144172776 | LY6E, Chr8: 144169776-144172776 |
| TRPM2 | Chr21: 44596411-44599411 | TRPM2, Chr21: 44596411-44599411 |
| GFAP | Chr17: 40346894-40349894 | GFAP, Chr17: 40346894-40349894 |
| BGN | Chr23: 152412104-152415104 | BGN, Chr23: 152412104-152415104 |
| GP9 | Chr3: 130260834-130263834 | GP9, Chr3: 130260834-130263834 |
| PCOLCE | Chr7: 100036317-100039317 | PCOLCE, Chr7: 100036317-100039317 |
| ODF3 | Chr11: 185260-188260 | ODF3, Chr11: 185260-188260 |
| MYO7A | Chr11: 76515457-76518457 | MYO7A, Chr11: 76515457-76518457 |
| MYO7A | Chr11: 76515463-76518463 | MYO7A, Chr11: 76515463-76518463 |
| SCNN1A | Chr12: 6353476-6356476 | SCNN1A, Chr12: 6353476-6356476 |
| GNB3 | Chr12: 6818135-6821135 | GNB3, Chr12: 6818135-6821135 |
| STUB1 | Chr16: 668615-671615 | STUB1, Chr16: 668615-671615 |
| KCNJ14 | Chr19: 53649077-53652077 | KCNJ14, Chr19: 53649077-53652077 |
| TMC4 | Chr19: 59367164-59370164 | TMC4, Chr19: 59367164-59370164 |
| ITGB2 | Chr21: 45171681-45174681 | ITGB2, Chr21: 45171681-45174681 |
| ACTRT2 | Chr1: 2926405-2929405 | ACTRT2, Chr1: 2926405-2929405 |
| LOC165186 | Chr2: 29056167-29059167 | LOC165186, Chr2: 29056167-29059167 |
| RET | Chr10: 42891022-42894022 | RET, Chr10: 42891022-42894022 |
| ACVRL1 | Chr12: 50590879-50593879 | ACVRL1, Chr12: 50590879-50593879 |
| PIGQ | Chr16: 558504-561504 | PIGQ, Chr16: 558504-561504 |
| TRPV1 | Chr17: 3444374-3447374 | TRPV1, Chr17: 3444374-3447374 |
| TPRX1 | Chr19: 52997173-53000173 | TPRX1, Chr19: 52997173-53000173 |
| AGRP | Chr16: 66073717-66076717 | AGRP, Chr16: 66073717-66076717 |
| C16orf14 | Chr16: 630349-633349 | C16orf14, Chr16: 630349-633349 |
| MGC29506 | Chr5: 138752004-138755004 | MGC29506, Chr5: 138752004-138755004 |
| EHMT1 | Chr9: 139723737-139726737 | EHMT1, Chr9: 139723737-139726737 |
| PLA2G4B | Chr15: 39916802-39919802 | PLA2G4B, Chr15: 39916802-39919802 |
| C16orf13 | Chr16: 624848-627848 | C16orf13, Chr16: 624848-627848 |
| QRFP | Chr9: 132757546-132760546 | QRFP, Chr9: 132757546-132760546 |
| TFR2 | Chr7: 100075609-100078609 | TFR2, Chr7: 100075609-100078609 |
| PRSS22 | Chr16: 2846672-2849672 | PRSS22, Chr16: 2846672-2849672 |
| ALDH3B1 | Chr11: 67532865-67535865 | ALDH3B1, Chr11: 67532865-67535865 |
| TFF3 | Chr21: 42607275-42610275 | TFF3, Chr21: 42607275-42610275 |
| LOC388419 | Chr17: 69868053-69871053 | LOC388419, Chr17: 69868053-69871053 |
| IL27 | Chr16: 28424156-28427156 | IL27, Chr16: 28424156-28427156 |
| FLJ45909 | Chr19: 44059234-44062234 | FLJ45909, Chr19: 44059234-44062234 |
| NRN1L | Chr16: 66474781-66477781 | NRN1L, Chr16: 66474781-66477781 |
| ANKRD2 | Chr10: 99320745-99323745 | ANKRD2, Chr10: 99320745-99323745 |
| CGB1 | Chr19: 54230503-54233503 | CGB1, Chr19: 54230503-54233503 |
| IL17B | Chr5: 148737531-148740531 | IL17B, Chr5: 148737531-148740531 |
| KRTAP5-4 | Chr11: 1598444-1601444 | KRTAP5-4, Chr11: 1598444-1601444 |
| SPAG7 | Chr17: 4810356-4813356 | SPAG7, Chr17: 4810356-4813356 |
| ACOT7 | Chr1: 6340091-6343091 | ACOT7, Chr1: 6340091-6343091 |
| PSORS1C2 | Chr6: 31213606-31216606 | PSORS1C2, Chr6: 31213606-31216606 |
| CALHM3 | Chr10: 105227487-105230487 | CALHM3, Chr10: 105227487-105230487 |
| NARFL | Chr16: 729498-732498 | NARFL, Chr16: 729498-732498 |
| HCRTR1 | Chr1: 31855727-31858727 | HCRTR1, Chr1: 31855727-31858727 |
| PPFIA4 | Chr1: 201285433-201288433 | PPFIA4, Chr1: 201285433-201288433 |
| C8orf31 | Chr8: 144190553-144193553 | C8orf31, Chr8: 144190553-144193553 |
| PCBP4 | Chr3: 51970448-51973448 | PCBP4, Chr3: 51970448-51973448 |
| ZMIZ2 | Chr7: 44760811-44763811 | ZMIZ2, Chr7: 44760811-44763811 |
| SEMA4D | Chr9: 91282931-91285931 | SEMA4D, Chr9: 91282931-91285931 |
| C13orf35 | Chr13: 112347858-112350858 | C13orf35, Chr13: 112347858-112350858 |
| HAGHL | Chr16: 715766-718766 | HAGHL, Chr16: 715766-718766 |
| DNASE1 | Chr16: 3641440-3644440 | DNASE1, Chr16: 3641440-3644440 |
| DDC | Chr7: 50594762-50597762 | DDC, Chr7: 50594762-50597762 |
| GPER | Chr7: 1092748-1095748 | GPER, Chr7: 1092748-1095748 |
| KCNN4 | Chr19: 48975749-48978749 | KCNN4, Chr19: 48975749-48978749 |
| C20orf114 | Chr20: 31333101-31336101 | C20orf114, Chr20: 31333101-31336101 |
| ATP6V1G2 | Chr6: 31621106-31624106 | ATP6V1G2, Chr6: 31621106-31624106 |
| C6orf26 | Chr6: 31837251-31840251 | C6orf26, Chr6: 31837251-31840251 |
| FBXL16 | Chr16: 694309-697309 | FBXL16, Chr16: 694309-697309 |
| HAGHL | Chr16: 715458-718458 | HAGHL, Chr16: 715458-718458 |
| SPEM1 | Chr17: 7262902-7265902 | SPEM1, Chr17: 7262902-7265902 |
| APOBEC3A | Chr22: 37682068-37685068 | APOBEC3A, Chr22: 37682068-37685068 |
| SLC7A9 | Chr19: 38051023-38054023 | SLC7A9, Chr19: 38051023-38054023 |
| GHRH | Chr20: 35317206-35320206 | GHRH, Chr20: 35317206-35320206 |
| CHRM1 | Chr11: 62444088-62447088 | CHRM1, Chr11: 62444088-62447088 |
| KRTAP5-8 | Chr11: 70925218-70928218 | KRTAP5-8, Chr11: 70925218-70928218 |
| C1QTNF9 | Chr13: 23780215-23783215 | C1QTNF9, Chr13: 23780215-23783215 |
| LAMB2 | Chr3: 49144103-49147103 | LAMB2, Chr3: 49144103-49147103 |
| SLC22A18 | Chr11: 2876026-2879026 | SLC22A18, Chr11: 2876026-2879026 |
| CD37 | Chr19: 54528988-54531988 | CD37, Chr19: 54528988-54531988 |
| LAMB3 | Chr1: 207889802-207892802 | LAMB3, Chr1: 207889802-207892802 |
| FLJ13137 | Chr1: 160616643-160619643 | FLJ13137, Chr1: 160616643-160619643 |
| TRPM1 | Chr15: 29179716-29182716 | TRPM1, Chr15: 29179716-29182716 |
| TNK2 | Chr3: 197118777-197121777 | TNK2, Chr3: 197118777-197121777 |
| TNFSF13 | Chr17: 7400839-7403839 | TNFSF13, Chr17: 7400839-7403839 |
| KIF25 | Chr6: 168159901-168162901 | KIF25, Chr6: 168159901-168162901 |
| PITPNM2 | Chr12: 122159428-122162428 | PITPNM2, Chr12: 122159428-122162428 |
| CTSL3 | Chr9: 89576149-89579149 | CTSL3, Chr9: 89576149-89579149 |
| MGC26718 | Chr18: 14167595-14170595 | MGC26718, Chr18: 14167595-14170595 |
| HSH2D | Chr19: 16104337-16107337 | HSH2D, Chr19: 16104337-16107337 |
| LOC284402 | Chr19: 39775830-39778830 | LOC284402, Chr19: 39775830-39778830 |
| UPK1A | Chr19: 40848054-40851054 | UPK1A, Chr19: 40848054-40851054 |
| PSG5 | Chr19: 48381028-48384028 | PSG5, Chr19: 48381028-48384028 |
| PSG6 | Chr19: 48112383-48115383 | PSG6, Chr19: 48112383-48115383 |
| PSG4 | Chr19: 48400130-48403130 | PSG4, Chr19: 48400130-48403130 |
| HOXC8 | Chr12: 52687656-52690656 | HOXC8, Chr12: 52687656-52690656 |
| ITGAX | Chr16: 31272509-31275509 | ITGAX, Chr16: 31272509-31275509 |
| DECR2 | Chr16: 390358-393358 | DECR2, Chr16: 390358-393358 |
| HIGD1C | Chr12: 49632548-49635548 | HIGD1C, Chr12: 49632548-49635548 |
| PSG6 | Chr19: 48112329-48115329 | PSG6, Chr19: 48112329-48115329 |
| PSG9 | Chr19: 48464022-48467022 | PSG9, Chr19: 48464022-48467022 |
| CGB5 | Chr19: 54237413-54240413 | CGB5, Chr19: 54237413-54240413 |
| NPFFR1 | Chr10: 71694654-71697654 | NPFFR1, Chr10: 71694654-71697654 |
| GGT1 | Chr22: 23327679-23330679 | GGT1, Chr22: 23327679-23330679 |
| FAM131A | Chr3: 185539451-185542451 | FAM131A, Chr3: 185539451-185542451 |
| RTN2 | Chr19: 50686814-50689814 | RTN2, Chr19: 50686814-50689814 |
| ELF5 | Chr11: 34488422-34491422 | ELF5, Chr11: 34488422-34491422 |
| LOC728137 | Chr24: 9974007-9977007 | LOC728137, Chr24: 9974007-9977007 |
| TMEM95 | Chr17: 7197720-7200720 | TMEM95, Chr17: 7197720-7200720 |
| METAP1 | Chr4: 100134310-100137310 | METAP1, Chr4: 100134310-100137310 |
| ASPA | Chr17: 3322653-3325653 | ASPA, Chr17: 3322653-3325653 |
| IRF5 | Chr7: 128366507-128369507 | IRF5, Chr7: 128366507-128369507 |
| C1orf222 | Chr1: 1847728-1850728 | C1orf222, Chr1: 1847728-1850728 |
| GRAMD4 | Chr22: 45399821-45402821 | GRAMD4, Chr22: 45399821-45402821 |
| FXYD1 | Chr19: 40320731-40323731 | FXYD1, Chr19: 40320731-40323731 |
| ACP5 | Chr19: 11548053-11551053 | ACP5, Chr19: 11548053-11551053 |
| NR2E3 | Chr15: 69888447-69891447 | NR2E3, Chr15: 69888447-69891447 |
| C14orf68 | Chr14: 99857931-99860931 | C14orf68, Chr14: 99857931-99860931 |
| IFFO | Chr12: 6527035-6530035 | IFFO, Chr12: 6527035-6530035 |
| A26A1 | Chr8: 43265241-43268241 | A26A1, Chr8: 43265241-43268241 |
| HHATL | Chr3: 42716517-42719517 | HHATL, Chr3: 42716517-42719517 |
| TREX1 | Chr3: 48480732-48483732 | TREX1, Chr3: 48480732-48483732 |
| ANK1 | Chr8: 41772797-41775797 | ANK1, Chr8: 41772797-41775797 |
| CACNG1 | Chr17: 62469667-62472667 | CACNG1, Chr17: 62469667-62472667 |
| DNMT3L | Chr21: 44505027-44508027 | DNMT3L, Chr21: 44505027-44508027 |
| CNTN2 | Chr1: 203277462-203280462 | CNTN2, Chr1: 203277462-203280462 |
| LDB3 | Chr10: 88416905-88419905 | LDB3, Chr10: 88416905-88419905 |
| DHRS7C | Chr17: 9633826-9636826 | DHRS7C, Chr17: 9633826-9636826 |
| TEX28 | Chr23: 153175132-153178132 | TEX28, Chr23: 153175132-153178132 |
| TMIE | Chr3: 46716326-46719326 | TMIE, Chr3: 46716326-46719326 |
| TUBB1 | Chr20: 57026203-57029203 | TUBB1, Chr20: 57026203-57029203 |
| C3orf67 | Chr3: 59009255-59012255 | C3orf67, Chr3: 59009255-59012255 |
| PLCB2 | Chr15: 38385966-38388966 | PLCB2, Chr15: 38385966-38388966 |
| RAPGEFL1 | Chr17: 35586267-35589267 | RAPGEFL1, Chr17: 35586267-35589267 |
| ZNF80 | Chr3: 115437615-115440615 | ZNF80, Chr3: 115437615-115440615 |
| SLC44A4 | Chr6: 31953302-31956302 | SLC44A4, Chr6: 31953302-31956302 |
| DNAJC4 | Chr11: 63752828-63755828 | DNAJC4, Chr11: 63752828-63755828 |
| EIF4A3 | Chr17: 75734033-75737033 | EIF4A3, Chr17: 75734033-75737033 |
| PCP4 | Chr21: 40159716-40162716 | PCP4, Chr21: 40159716-40162716 |
| DNASE1L3 | Chr3: 58173938-58176938 | DNASE1L3, Chr3: 58173938-58176938 |
| CRYBA1 | Chr17: 24596500-24599500 | CRYBA1, Chr17: 24596500-24599500 |
| MSLN | Chr16: 749633-752633 | MSLN, Chr16: 749633-752633 |
| CLDN19 | Chr1: 42977012-42980012 | CLDN19, Chr1: 42977012-42980012 |
| GPR20 | Chr8: 142445047-142448047 | GPR20, Chr8: 142445047-142448047 |
| PPP1R16A | Chr8: 145691416-145694416 | PPP1R16A, Chr8: 145691416-145694416 |
| TREX2 | Chr23: 152363639-152366639 | TREX2, Chr23: 152363639-152366639 |
| SRPK3 | Chr23: 152698203-152701203 | SRPK3, Chr23: 152698203-152701203 |
| AQP12A | Chr2: 241278434-241281434 | AQP12A, Chr2: 241278434-241281434 |
| ELMO3 | Chr16: 65789028-65792028 | ELMO3, Chr16: 65789028-65792028 |
| KIAA1881 | Chr19: 4467216-4470216 | KIAA1881, Chr19: 4467216-4470216 |
| SLC23A1 | Chr5: 138745400-138748400 | SLC23A1, Chr5: 138745400-138748400 |
| CTAG2 | Chr23: 153533535-153536535 | CTAG2, Chr23: 153533535-153536535 |
| CTAG2 | Chr23: 153533536-153536536 | CTAG2, Chr23: 153533536-153536536 |
| EPB49 | Chr8: 21966773-21969773 | EPB49, Chr8: 21966773-21969773 |
| PHYHIP | Chr8: 22144296-22147296 | PHYHIP, Chr8: 22144296-22147296 |
| C14orf48 | Chr14: 93531894-93534894 | C14orf48, Chr14: 93531894-93534894 |
| CTF1 | Chr16: 30813928-30816928 | CTF1, Chr16: 30813928-30816928 |
| GP5 | Chr3: 195599784-195602784 | GP5, Chr3: 195599784-195602784 |
| ANK1 | Chr8: 41640741-41643741 | ANK1, Chr8: 41640741-41643741 |
| GIMAP7 | Chr7: 149841377-149844377 | GIMAP7, Chr7: 149841377-149844377 |
| EPPK1 | Chr8: 145017922-145020922 | EPPK1, Chr8: 145017922-145020922 |
| LCN6 | Chr9: 138761301-138764301 | LCN6, Chr9: 138761301-138764301 |
| PRAP1 | Chr10: 135009400-135012400 | PRAP1, Chr10: 135009400-135012400 |
| ATP4B | Chr13: 113359002-113362002 | ATP4B, Chr13: 113359002-113362002 |
| CRYAA | Chr21: 43460709-43463709 | CRYAA, Chr21: 43460709-43463709 |
| FAM116B | Chr22: 49098510-49101510 | FAM116B, Chr22: 49098510-49101510 |
| MIOX | Chr22: 49270578-49273578 | MIOX, Chr22: 49270578-49273578 |
| HGFAC | Chr4: 3412023-3415023 | HGFAC, Chr4: 3412023-3415023 |
| MRGPRF | Chr11: 68535926-68538926 | MRGPRF, Chr11: 68535926-68538926 |
| PPAPDC3 | Chr9: 133153444-133156444 | PPAPDC3, Chr9: 133153444-133156444 |
| SPTBN5 | Chr15: 39972067-39975067 | SPTBN5, Chr15: 39972067-39975067 |
| CHRND | Chr2: 233097665-233100665 | CHRND, Chr2: 233097665-233100665 |
| PLD4 | Chr14: 104460731-104463731 | PLD4, Chr14: 104460731-104463731 |
| SLFNL1 | Chr1: 41258474-41261474 | SLFNL1, Chr1: 41258474-41261474 |
| OBP2A | Chr9: 137576305-137579305 | OBP2A, Chr9: 137576305-137579305 |
| AQP12B | Chr2: 241269490-241272490 | AQP12B, Chr2: 241269490-241272490 |
| WIPI2 | Chr7: 5218925-5221925 | WIPI2, Chr7: 5218925-5221925 |
| CDSN | Chr6: 31194702-31197702 | CDSN, Chr6: 31194702-31197702 |
| RTP4 | Chr3: 188567361-188570361 | RTP4, Chr3: 188567361-188570361 |
| FAM57B | Chr16: 29948187-29951187 | FAM57B, Chr16: 29948187-29951187 |
| SH2D6 | Chr2: 85513928-85516928 | SH2D6, Chr2: 85513928-85516928 |
| AQP2 | Chr12: 48629295-48632295 | AQP2, Chr12: 48629295-48632295 |
| KCNN2 | Chr5: 113795625-113798625 | KCNN2, Chr5: 113795625-113798625 |
| TCIRG1 | Chr11: 67561558-67564558 | TCIRG1, Chr11: 67561558-67564558 |
| IGSF11 | Chr3: 120346088-120349088 | IGSF11, Chr3: 120346088-120349088 |
| CPXCR1 | Chr23: 87887381-87890381 | CPXCR1, Chr23: 87887381-87890381 |
| SCN11A | Chr3: 38965556-38968556 | SCN11A, Chr3: 38965556-38968556 |
| KIAA1026 | Chr1: 15127382-15130382 | KIAA1026, Chr1: 15127382-15130382 |
| TTC4 | Chr1: 54952616-54955616 | TTC4, Chr1: 54952616-54955616 |
| CYP2W1 | Chr7: 987860-990860 | CYP2W1, Chr7: 987860-990860 |
| ALPPL2 | Chr2: 232978295-232981295 | ALPPL2, Chr2: 232978295-232981295 |
| CPA1 | Chr7: 129806090-129809090 | CPA1, Chr7: 129806090-129809090 |
| KRTAP10-6 | Chr21: 44835314-44838314 | KRTAP10-6, Chr21: 44835314-44838314 |
| CCDC116 | Chr22: 20315585-20318585 | CCDC116, Chr22: 20315585-20318585 |
| KRTAP10-2 | Chr21: 44794316-44797316 | KRTAP10-2, Chr21: 44794316-44797316 |
| CDK3 | Chr17: 71507081-71510081 | CDK3, Chr17: 71507081-71510081 |
| PTPN7 | Chr1: 200394874-200397874 | PTPN7, Chr1: 200394874-200397874 |
| UNQ2541 | Chr9: 138777286-138780286 | UNQ2541, Chr9: 138777286-138780286 |
| ELN | Chr7: 73078862-73081862 | ELN, Chr7: 73078862-73081862 |
| BGLAP | Chr1: 154477074-154480074 | BGLAP, Chr1: 154477074-154480074 |
| C9orf164 | Chr9: 91167886-91170886 | C9orf164, Chr9: 91167886-91170886 |
| FAM83E | Chr19: 53807006-53810006 | FAM83E, Chr19: 53807006-53810006 |
| IL32 | Chr16: 3054285-3057285 | IL32, Chr16: 3054285-3057285 |
| MYL7 | Chr7: 44145941-44148941 | MYL7, Chr7: 44145941-44148941 |
| OXER1 | Chr2: 42843405-42846405 | OXER1, Chr2: 42843405-42846405 |
| SIX3 | Chr2: 45021040-45024040 | SIX3, Chr2: 45021040-45024040 |
| SLC27A5 | Chr19: 63713657-63716657 | SLC27A5, Chr19: 63713657-63716657 |
| C20orf59 | Chr20: 61052943-61055943 | C20orf59, Chr20: 61052943-61055943 |
| MUC4 | Chr3: 197022045-197025045 | MUC4, Chr3: 197022045-197025045 |
| SLC47A2 | Chr17: 19559135-19562135 | SLC47A2, Chr17: 19559135-19562135 |
| UROC1 | Chr3: 127717784-127720784 | UROC1, Chr3: 127717784-127720784 |
| ZNF232 | Chr17: 4965621-4968621 | ZNF232, Chr17: 4965621-4968621 |
| NDST1 | Chr5: 149879122-149882122 | NDST1, Chr5: 149879122-149882122 |
| WDR24 | Chr16: 678901-681901 | WDR24, Chr16: 678901-681901 |
| KIAA1644 | Chr22: 43038564-43041564 | KIAA1644, Chr22: 43038564-43041564 |
| HHIP | Chr4: 145785122-145788122 | HHIP, Chr4: 145785122-145788122 |
| KLK6 | Chr19: 56162359-56165359 | KLK6, Chr19: 56162359-56165359 |
| OPN1LW | Chr23: 153061418-153064418 | OPN1LW, Chr23: 153061418-153064418 |
| NUDT18 | Chr8: 22021377-22024377 | NUDT18, Chr8: 22021377-22024377 |
| GNA15 | Chr19: 3085690-3088690 | GNA15, Chr19: 3085690-3088690 |
| A26B3 | Chr21: 13902868-13905868 | A26B3, Chr21: 13902868-13905868 |
| FXYD4 | Chr10: 43185597-43188597 | FXYD4, Chr10: 43185597-43188597 |
| SLC39A12 | Chr10: 18279326-18282326 | SLC39A12, Chr10: 18279326-18282326 |
| ALPP | Chr2: 232950091-232953091 | ALPP, Chr2: 232950091-232953091 |
| LOC441476 | Chr9: 139264050-139267050 | LOC441476, Chr9: 139264050-139267050 |
| KRTAP10-9 | Chr21: 44869967-44872967 | KRTAP10-9, Chr21: 44869967-44872967 |
| FTCD | Chr21: 46398409-46401409 | FTCD, Chr21: 46398409-46401409 |
| SNCG | Chr10: 88706892-88709892 | SNCG, Chr10: 88706892-88709892 |
| ATP2B3 | Chr23: 152453273-152456273 | ATP2B3, Chr23: 152453273-152456273 |
| LOC143678 | Chr11: 45883909-45886909 | LOC143678, Chr11: 45883909-45886909 |
| CD93 | Chr20: 23013477-23016477 | CD93, Chr20: 23013477-23016477 |
| RUNX3 | Chr1: 25162588-25165588 | RUNX3, Chr1: 25162588-25165588 |
| IL32 | Chr16: 3054163-3057163 | IL32, Chr16: 3054163-3057163 |
| IFITM5 | Chr11: 288026-291026 | IFITM5, Chr11: 288026-291026 |
| FOXN1 | Chr17: 23873585-23876585 | FOXN1, Chr17: 23873585-23876585 |
| IL32 | Chr16: 3054139-3057139 | IL32, Chr16: 3054139-3057139 |
| TRPV3 | Chr17: 3406539-3409539 | TRPV3, Chr17: 3406539-3409539 |
| C13orf16 | Chr13: 110769515-110772515 | C13orf16, Chr13: 110769515-110772515 |
| MFNG | Chr22: 36210831-36213831 | MFNG, Chr22: 36210831-36213831 |
| ZAP70 | Chr2: 97694962-97697962 | ZAP70, Chr2: 97694962-97697962 |
| COL24A1 | Chr1: 86393209-86396209 | COL24A1, Chr1: 86393209-86396209 |
| R3HDML | Chr20: 42397539-42400539 | R3HDML, Chr20: 42397539-42400539 |
| GDF2 | Chr10: 48035359-48038359 | GDF2, Chr10: 48035359-48038359 |
| ACE | Chr17: 58914408-58917408 | ACE, Chr17: 58914408-58917408 |
| DUSP13 | Chr10: 76527754-76530754 | DUSP13, Chr10: 76527754-76530754 |
| WDFY4 | Chr10: 49562023-49565023 | WDFY4, Chr10: 49562023-49565023 |
| C3orf27 | Chr3: 129776119-129779119 | C3orf27, Chr3: 129776119-129779119 |
| SCMH1 | Chr1: 41398133-41401133 | SCMH1, Chr1: 41398133-41401133 |
| GBP5 | Chr1: 89509619-89512619 | GBP5, Chr1: 89509619-89512619 |
| SPDEF | Chr6: 34630569-34633569 | SPDEF, Chr6: 34630569-34633569 |
| P2RX1 | Chr17: 3765209-3768209 | P2RX1, Chr17: 3765209-3768209 |
| HOXD9 | Chr2: 176693833-176696833 | HOXD9, Chr2: 176693833-176696833 |
| MUC5AC | Chr11: 1130973-1133973 | MUC5AC, Chr11: 1130973-1133973 |
| CALHM1 | Chr10: 105207135-105210135 | CALHM1, Chr10: 105207135-105210135 |
| ALOX12 | Chr17: 6838607-6841607 | ALOX12, Chr17: 6838607-6841607 |
| PTK6 | Chr20: 61637651-61640651 | PTK6, Chr20: 61637651-61640651 |
| IL32 | Chr16: 3054112-3057112 | IL32, Chr16: 3054112-3057112 |
| AANAT | Chr17: 71973745-71976745 | AANAT, Chr17: 71973745-71976745 |
| PASD1 | Chr23: 150481162-150484162 | PASD1, Chr23: 150481162-150484162 |
| PDLIM4 | Chr5: 131619785-131622785 | PDLIM4, Chr5: 131619785-131622785 |
| L3MBTL | Chr20: 41574966-41577966 | L3MBTL, Chr20: 41574966-41577966 |
| AGPAT3 | Chr21: 44168206-44171206 | AGPAT3, Chr21: 44168206-44171206 |
| RNF213 | Chr17: 75926820-75929820 | RNF213, Chr17: 75926820-75929820 |
| MAG | Chr19: 40473377-40476377 | MAG, Chr19: 40473377-40476377 |
| GATA1 | Chr23: 48528425-48531425 | GATA1, Chr23: 48528425-48531425 |
| FAM58B | Chr1: 198447778-198450778 | FAM58B, Chr1: 198447778-198450778 |
| C10orf109 | Chr10: 1557324-1560324 | C10orf109, Chr10: 1557324-1560324 |
| ETV7 | Chr6: 36461945-36464945 | ETV7, Chr6: 36461945-36464945 |
| ACCN1 | Chr17: 28642619-28645619 | ACCN1, Chr17: 28642619-28645619 |
| PPP1R2 | Chr3: 196749862-196752862 | PPP1R2, Chr3: 196749862-196752862 |
| DOK3 | Chr5: 176867959-176870959 | DOK3, Chr5: 176867959-176870959 |
| FXYD1 | Chr19: 40320071-40323071 | FXYD1, Chr19: 40320071-40323071 |
| LOC441956 | Chr21: 13973830-13976830 | LOC441956, Chr21: 13973830-13976830 |
| KCNAB2 | Chr1: 6007466-6010466 | KCNAB2, Chr1: 6007466-6010466 |
| C7orf29 | Chr7: 149656370-149659370 | C7orf29, Chr7: 149656370-149659370 |
| OTOS | Chr2: 240727246-240730246 | OTOS, Chr2: 240727246-240730246 |
| ANKRD23 | Chr2: 96871985-96874985 | ANKRD23, Chr2: 96871985-96874985 |
| KRT73 | Chr12: 51297110-51300110 | KRT73, Chr12: 51297110-51300110 |
| CENTA1 | Chr7: 959315-962315 | CENTA1, Chr7: 959315-962315 |
| LOC646643 | Chr19: 60737973-60740973 | LOC646643, Chr19: 60737973-60740973 |
| CORT | Chr1: 10431057-10434057 | CORT, Chr1: 10431057-10434057 |
| RBPJL | Chr20: 43367404-43370404 | RBPJL, Chr20: 43367404-43370404 |
| A1BG | Chr19: 63555177-63558177 | A1BG, Chr19: 63555177-63558177 |
| CHST5 | Chr16: 74125069-74128069 | CHST5, Chr16: 74125069-74128069 |
| FGFBP2 | Chr4: 15572426-15575426 | FGFBP2, Chr4: 15572426-15575426 |
| SP7 | Chr12: 52014304-52017304 | SP7, Chr12: 52014304-52017304 |
| LOC497190 | Chr16: 73011369-73014369 | LOC497190, Chr16: 73011369-73014369 |
| CYTL1 | Chr4: 5070598-5073598 | CYTL1, Chr4: 5070598-5073598 |
| KIAA1210 | Chr23: 118167070-118170070 | KIAA1210, Chr23: 118167070-118170070 |
| GML | Chr8: 143911718-143914718 | GML, Chr8: 143911718-143914718 |
| C3orf56 | Chr3: 128393163-128396163 | C3orf56, Chr3: 128393163-128396163 |
| DAO | Chr12: 107796485-107799485 | DAO, Chr12: 107796485-107799485 |
| CEACAM16 | Chr19: 49892697-49895697 | CEACAM16, Chr19: 49892697-49895697 |
| AQP3 | Chr9: 33436090-33439090 | AQP3, Chr9: 33436090-33439090 |
| CALML6 | Chr1: 1834625-1837625 | CALML6, Chr1: 1834625-1837625 |
| GIGYF1 | Chr7: 100123306-100126306 | GIGYF1, Chr7: 100123306-100126306 |
| MCF2L | Chr13: 112680155-112683155 | MCF2L, Chr13: 112680155-112683155 |
| KRTAP10-12 | Chr21: 44940014-44943014 | KRTAP10-12, Chr21: 44940014-44943014 |
| C21orf56 | Chr21: 46427229-46430229 | C21orf56, Chr21: 46427229-46430229 |
| C2orf48 | Chr2: 10197459-10200459 | C2orf48, Chr2: 10197459-10200459 |
| ASB10 | Chr7: 150513911-150516911 | ASB10, Chr7: 150513911-150516911 |
| PAEP | Chr9: 137591924-137594924 | PAEP, Chr9: 137591924-137594924 |
| FBRS | Chr16: 30581778-30584778 | FBRS, Chr16: 30581778-30584778 |
| SSTR3 | Chr22: 35936799-35939799 | SSTR3, Chr22: 35936799-35939799 |
| FXYD2 | Chr11: 117199169-117202169 | FXYD2, Chr11: 117199169-117202169 |
| TTYH2 | Chr17: 69754599-69757599 | TTYH2, Chr17: 69754599-69757599 |
| ECE1 | Chr1: 21477270-21480270 | ECE1, Chr1: 21477270-21480270 |
| JAKMIP3 | Chr10: 133766802-133769802 | JAKMIP3, Chr10: 133766802-133769802 |
| EDN2 | Chr1: 41721384-41724384 | EDN2, Chr1: 41721384-41724384 |
| OMP | Chr11: 76490033-76493033 | OMP, Chr11: 76490033-76493033 |
| FCGRT | Chr19: 54706803-54709803 | FCGRT, Chr19: 54706803-54709803 |
| RASGRF1 | Chr15: 77083557-77086557 | RASGRF1, Chr15: 77083557-77086557 |
| PRTN3 | Chr19: 790484-793484 | PRTN3, Chr19: 790484-793484 |
| IGFN1 | Chr1: 199446892-199449892 | IGFN1, Chr1: 199446892-199449892 |
| CPSF4L | Chr17: 68768114-68771114 | CPSF4L, Chr17: 68768114-68771114 |
| TFDP3 | Chr23: 132178542-132181542 | TFDP3, Chr23: 132178542-132181542 |
| AQP7 | Chr9: 33391017-33394017 | AQP7, Chr9: 33391017-33394017 |
| SYNGR1 | Chr22: 38088620-38091620 | SYNGR1, Chr22: 38088620-38091620 |
| PI4KA | Chr22: 19417455-19420455 | PI4KA, Chr22: 19417455-19420455 |
| TGM3 | Chr20: 2223112-2226112 | TGM3, Chr20: 2223112-2226112 |
| PLXNB1 | Chr3: 48440246-48443246 | PLXNB1, Chr3: 48440246-48443246 |
| DGKZ | Chr11: 46338220-46341220 | DGKZ, Chr11: 46338220-46341220 |
| DPEP1 | Chr16: 88213000-88216000 | DPEP1, Chr16: 88213000-88216000 |
| PCDH24 | Chr5: 175907470-175910470 | PCDH24, Chr5: 175907470-175910470 |
| CD248 | Chr11: 65839591-65842591 | CD248, Chr11: 65839591-65842591 |
| KRTAP10-1 | Chr21: 44783006-44786006 | KRTAP10-1, Chr21: 44783006-44786006 |
| MLC1 | Chr22: 48864408-48867408 | MLC1, Chr22: 48864408-48867408 |
| ADPRHL1 | Chr13: 113154340-113157340 | ADPRHL1, Chr13: 113154340-113157340 |
| IL32 | Chr16: 3053813-3056813 | IL32, Chr16: 3053813-3056813 |
| SCAF1 | Chr19: 54838588-54841588 | SCAF1, Chr19: 54838588-54841588 |
| GSTM5 | Chr1: 110054887-110057887 | GSTM5, Chr1: 110054887-110057887 |
| RGS3 | Chr9: 115365681-115368681 | RGS3, Chr9: 115365681-115368681 |
| FBLIM1 | Chr1: 15962545-15965545 | FBLIM1, Chr1: 15962545-15965545 |
| SLC22A7 | Chr6: 43372475-43375475 | SLC22A7, Chr6: 43372475-43375475 |
| CCNI2 | Chr5: 132109535-132112535 | CCNI2, Chr5: 132109535-132112535 |
| MGC87631 | Chr17: 20738545-20741545 | MGC87631, Chr17: 20738545-20741545 |
| PISD | Chr22: 30355310-30358310 | PISD, Chr22: 30355310-30358310 |
| TNFSF14 | Chr19: 6620099-6623099 | TNFSF14, Chr19: 6620099-6623099 |
| SLC45A2 | Chr5: 34019037-34022037 | SLC45A2, Chr5: 34019037-34022037 |
| SPO11 | Chr20: 55336737-55339737 | SPO11, Chr20: 55336737-55339737 |
| CDH5 | Chr16: 64956525-64959525 | CDH5, Chr16: 64956525-64959525 |
| CLDN18 | Chr3: 139210195-139213195 | CLDN18, Chr3: 139210195-139213195 |
| A26B1 | Chr15: 19335167-19338167 | A26B1, Chr15: 19335167-19338167 |
| CD5 | Chr11: 60625005-60628005 | CD5, Chr11: 60625005-60628005 |
| SULT2B1 | Chr19: 53745740-53748740 | SULT2B1, Chr19: 53745740-53748740 |
| UBE2U | Chr1: 64440577-64443577 | UBE2U, Chr1: 64440577-64443577 |
| DAZ4 | Chr24: 25387951-25390951 | DAZ4, Chr24: 25387951-25390951 |
| DAZ2 | Chr24: 25387953-25390953 | DAZ2, Chr24: 25387953-25390953 |
| DAZ4 | Chr24: 25387968-25390968 | DAZ4, Chr24: 25387968-25390968 |
| DAZ3 | Chr24: 25387976-25390976 | DAZ3, Chr24: 25387976-25390976 |
| FAM110A | Chr20: 763210-766210 | FAM110A, Chr20: 763210-766210 |
| SLURP1 | Chr8: 143819331-143822331 | SLURP1, Chr8: 143819331-143822331 |
| NR5A1 | Chr9: 126308020-126311020 | NR5A1, Chr9: 126308020-126311020 |
| C9orf169 | Chr9: 139237964-139240964 | C9orf169, Chr9: 139237964-139240964 |
| C15orf52 | Chr15: 38418960-38421960 | C15orf52, Chr15: 38418960-38421960 |
| SLC6A9 | Chr1: 44254084-44257084 | SLC6A9, Chr1: 44254084-44257084 |
| NPC1L1 | Chr7: 44545939-44548939 | NPC1L1, Chr7: 44545939-44548939 |
| KRTAP10-10 | Chr21: 44880200-44883200 | KRTAP10-10, Chr21: 44880200-44883200 |
| UPK3B | Chr7: 75976180-75979180 | UPK3B, Chr7: 75976180-75979180 |
| SLC22A8 | Chr11: 62538387-62541387 | SLC22A8, Chr11: 62538387-62541387 |
| PGLYRP1 | Chr19: 51216663-51219663 | PGLYRP1, Chr19: 51216663-51219663 |
| FAM9A | Chr23: 8727924-8730924 | FAM9A, Chr23: 8727924-8730924 |
| PSTPIP1 | Chr15: 75073019-75076019 | PSTPIP1, Chr15: 75073019-75076019 |
| TTLL2 | Chr6: 167657063-167660063 | TTLL2, Chr6: 167657063-167660063 |
| IL34 | Chr16: 69236468-69239468 | IL34, Chr16: 69236468-69239468 |
| DKFZp564N2472 | Chr7: 53069342-53072342 | DKFZp564N2472, Chr7: 53069342-53072342 |
| TMEM89 | Chr3: 48632693-48635693 | TMEM89, Chr3: 48632693-48635693 |
| KRT3 | Chr12: 51474659-51477659 | KRT3, Chr12: 51474659-51477659 |
| ISG20 | Chr15: 86981542-86984542 | ISG20, Chr15: 86981542-86984542 |
| SPACA4 | Chr19: 53800311-53803311 | SPACA4, Chr19: 53800311-53803311 |
| TKTL1 | Chr23: 153175844-153178844 | TKTL1, Chr23: 153175844-153178844 |
| SFTPB | Chr2: 85747320-85750320 | SFTPB, Chr2: 85747320-85750320 |
| SFTPB | Chr2: 85747323-85750323 | SFTPB, Chr2: 85747323-85750323 |
| LOC348174 | Chr16: 73011650-73014650 | LOC348174, Chr16: 73011650-73014650 |
| CPNE6 | Chr14: 23609095-23612095 | CPNE6, Chr14: 23609095-23612095 |
| RPS6KA1 | Chr1: 26743429-26746429 | RPS6KA1, Chr1: 26743429-26746429 |
| MSH4 | Chr1: 76033717-76036717 | MSH4, Chr1: 76033717-76036717 |
| IGLL1 | Chr22: 22250995-22253995 | IGLL1, Chr22: 22250995-22253995 |
| EVPL | Chr17: 71533602-71536602 | EVPL, Chr17: 71533602-71536602 |
| ACTG2 | Chr2: 73972100-73975100 | ACTG2, Chr2: 73972100-73975100 |
| GPER | Chr7: 1091468-1094468 | GPER, Chr7: 1091468-1094468 |
| DCTN1 | Chr2: 74453487-74456487 | DCTN1, Chr2: 74453487-74456487 |
| C3orf30 | Chr3: 120346186-120349186 | C3orf30, Chr3: 120346186-120349186 |
| DNAJC5G | Chr2: 27350292-27353292 | DNAJC5G, Chr2: 27350292-27353292 |
| PIWIL4 | Chr11: 93938681-93941681 | PIWIL4, Chr11: 93938681-93941681 |
| CLEC9A | Chr12: 10073042-10076042 | CLEC9A, Chr12: 10073042-10076042 |
| LAMB3 | Chr1: 207890797-207893797 | LAMB3, Chr1: 207890797-207893797 |
| ADAM18 | Chr8: 39559798-39562798 | ADAM18, Chr8: 39559798-39562798 |
| TSPAN1 | Chr1: 46417298-46420298 | TSPAN1, Chr1: 46417298-46420298 |
| MRVI1 | Chr11: 10670181-10673181 | MRVI1, Chr11: 10670181-10673181 |
| ST18 | Chr8: 53483356-53486356 | ST18, Chr8: 53483356-53486356 |
| PCDHGC5 | Chr5: 140847491-140850491 | PCDHGC5, Chr5: 140847491-140850491 |
| RNASE7 | Chr14: 20578724-20581724 | RNASE7, Chr14: 20578724-20581724 |
| CIB4 | Chr2: 26716215-26719215 | CIB4, Chr2: 26716215-26719215 |
| ALDH1L2 | Chr12: 104000866-104003866 | ALDH1L2, Chr12: 104000866-104003866 |
| DAZ2 | Chr24: 25387895-25390895 | DAZ2, Chr24: 25387895-25390895 |
| KLHDC7A | Chr1: 18678510-18681510 | KLHDC7A, Chr1: 18678510-18681510 |
| FLRT1 | Chr11: 63626437-63629437 | FLRT1, Chr11: 63626437-63629437 |
| GLP-1 | Chr19: 10279733-10282733 | GLP-1, Chr19: 10279733-10282733 |
| PROM2 | Chr2: 95302427-95305427 | PROM2, Chr2: 95302427-95305427 |
| C2orf57 | Chr2: 232164355-232167355 | C2orf57, Chr2: 232164355-232167355 |
| ZFAT | Chr8: 135776476-135779476 | ZFAT, Chr8: 135776476-135779476 |
| RAPSN | Chr11: 47425806-47428806 | RAPSN, Chr11: 47425806-47428806 |
| SLC22A12 | Chr11: 64113357-64116357 | SLC22A12, Chr11: 64113357-64116357 |
| C12orf12 | Chr12: 89871584-89874584 | C12orf12, Chr12: 89871584-89874584 |
| SGCA | Chr17: 45596889-45599889 | SGCA, Chr17: 45596889-45599889 |
| CACNA1I | Chr22: 38295203-38298203 | CACNA1I, Chr22: 38295203-38298203 |
| DAZL | Chr3: 16620510-16623510 | DAZL, Chr3: 16620510-16623510 |
| GLIS2 | Chr16: 4320725-4323725 | GLIS2, Chr16: 4320725-4323725 |
| PTPN6 | Chr12: 6929194-6932194 | PTPN6, Chr12: 6929194-6932194 |
| TRPV1 | Chr17: 3441420-3444420 | TRPV1, Chr17: 3441420-3444420 |
| KRTAP10-8 | Chr21: 44854923-44857923 | KRTAP10-8, Chr21: 44854923-44857923 |
| FAM9B | Chr23: 8960668-8963668 | FAM9B, Chr23: 8960668-8963668 |
| GPR175 | Chr3: 128780470-128783470 | GPR175, Chr3: 128780470-128783470 |
| MPN2 | Chr1: 226068540-226071540 | MPN2, Chr1: 226068540-226071540 |
| CYP27C1 | Chr2: 127678313-127681313 | CYP27C1, Chr2: 127678313-127681313 |
| FAM166A | Chr9: 139260543-139263543 | FAM166A, Chr9: 139260543-139263543 |
| FAM83C | Chr20: 33342139-33345139 | FAM83C, Chr20: 33342139-33345139 |
| EFCAB4B | Chr12: 3731024-3734024 | EFCAB4B, Chr12: 3731024-3734024 |
| ACPT | Chr19: 55983983-55986983 | ACPT, Chr19: 55983983-55986983 |
| PRSSL1 | Chr19: 644961-647961 | PRSSL1, Chr19: 644961-647961 |
| CST7 | Chr20: 24876365-24879365 | CST7, Chr20: 24876365-24879365 |
| ACTC1 | Chr15: 32873719-32876719 | ACTC1, Chr15: 32873719-32876719 |
| LOC133874 | Chr5: 169591027-169594027 | LOC133874, Chr5: 169591027-169594027 |
| STRA6 | Chr15: 72280745-72283745 | STRA6, Chr15: 72280745-72283745 |
| HNF4A | Chr20: 42461837-42464837 | HNF4A, Chr20: 42461837-42464837 |
| SLC45A1 | Chr1: 8305476-8308476 | SLC45A1, Chr1: 8305476-8308476 |
| UNQ830 | Chr2: 233441737-233444737 | UNQ830, Chr2: 233441737-233444737 |
| GRK7 | Chr3: 142978232-142981232 | GRK7, Chr3: 142978232-142981232 |
| SYCP3 | Chr12: 100655878-100658878 | SYCP3, Chr12: 100655878-100658878 |
| NRIP2 | Chr12: 2812950-2815950 | NRIP2, Chr12: 2812950-2815950 |
| KLF17 | Chr1: 44355608-44358608 | KLF17, Chr1: 44355608-44358608 |
| RGS14 | Chr5: 176715949-176718949 | RGS14, Chr5: 176715949-176718949 |
| SOHLH2 | Chr13: 35685176-35688176 | SOHLH2, Chr13: 35685176-35688176 |
| EXOC3L | Chr16: 65780108-65783108 | EXOC3L, Chr16: 65780108-65783108 |
| C20orf118 | Chr20: 34936483-34939483 | C20orf118, Chr20: 34936483-34939483 |
| AIPL1 | Chr17: 6277743-6280743 | AIPL1, Chr17: 6277743-6280743 |
| PSD4 | Chr2: 113646530-113649530 | PSD4, Chr2: 113646530-113649530 |
| OVCH1 | Chr12: 29540386-29543386 | OVCH1, Chr12: 29540386-29543386 |
| LCK | Chr1: 32487926-32490926 | LCK, Chr1: 32487926-32490926 |
| SPATC1 | Chr8: 145157094-145160094 | SPATC1, Chr8: 145157094-145160094 |
| LOC389827 | Chr9: 135378287-135381287 | LOC389827, Chr9: 135378287-135381287 |
| ZP1 | Chr11: 60390090-60393090 | ZP1, Chr11: 60390090-60393090 |
| CTSW | Chr11: 65402359-65405359 | CTSW, Chr11: 65402359-65405359 |
| KRTAP12-3 | Chr21: 44900776-44903776 | KRTAP12-3, Chr21: 44900776-44903776 |
| CCL25 | Chr19: 8022433-8025433 | CCL25, Chr19: 8022433-8025433 |
| CD6 | Chr11: 60494190-60497190 | CD6, Chr11: 60494190-60497190 |
| GK2 | Chr4: 80546896-80549896 | GK2, Chr4: 80546896-80549896 |
| ADAM2 | Chr8: 39813436-39816436 | ADAM2, Chr8: 39813436-39816436 |
| LOC283677 | Chr15: 71521051-71524051 | LOC283677, Chr15: 71521051-71524051 |
| WWP2 | Chr16: 68514901-68517901 | WWP2, Chr16: 68514901-68517901 |
| PSMA8 | Chr18: 21966313-21969313 | PSMA8, Chr18: 21966313-21969313 |
| TMPRSS9 | Chr19: 2339283-2342283 | TMPRSS9, Chr19: 2339283-2342283 |
| SLC6A16 | Chr19: 54518786-54521786 | SLC6A16, Chr19: 54518786-54521786 |
| SASH3 | Chr23: 128740140-128743140 | SASH3, Chr23: 128740140-128743140 |
| CLEC3B | Chr3: 45041262-45044262 | CLEC3B, Chr3: 45041262-45044262 |
| CHCHD9 | Chr9: 81194513-81197513 | CHCHD9, Chr9: 81194513-81197513 |
| SLC25A24 | Chr1: 108535386-108538386 | SLC25A24, Chr1: 108535386-108538386 |
| CD19 | Chr16: 28849260-28852260 | CD19, Chr16: 28849260-28852260 |
| SNRPN | Chr15: 22618386-22621386 | SNRPN, Chr15: 22618386-22621386 |
| ASAH3 | Chr19: 6283062-6286062 | ASAH3, Chr19: 6283062-6286062 |
| ANGPTL6 | Chr19: 10072925-10075925 | ANGPTL6, Chr19: 10072925-10075925 |
| FCHO1 | Chr19: 17721835-17724835 | FCHO1, Chr19: 17721835-17724835 |
| PDHA2 | Chr4: 96978761-96981761 | PDHA2, Chr4: 96978761-96981761 |
| GAGE2B | Chr23: 49121151-49124151 | GAGE2B, Chr23: 49121151-49124151 |
| RAB41 | Chr23: 69417292-69420292 | RAB41, Chr23: 69417292-69420292 |
| GCK | Chr7: 44163912-44166912 | GCK, Chr7: 44163912-44166912 |
| CAPS | Chr19: 5863692-5866692 | CAPS, Chr19: 5863692-5866692 |
| KRTAP10-3 | Chr21: 44801571-44804571 | KRTAP10-3, Chr21: 44801571-44804571 |
| CHRNG | Chr2: 233111180-233114180 | CHRNG, Chr2: 233111180-233114180 |
| CCDC88B | Chr11: 63862770-63865770 | CCDC88B, Chr11: 63862770-63865770 |
| PAK6 | Chr15: 38317826-38320826 | PAK6, Chr15: 38317826-38320826 |
| PDE6G | Chr17: 77232538-77235538 | PDE6G, Chr17: 77232538-77235538 |
| SMARCA4 | Chr19: 10954327-10957327 | SMARCA4, Chr19: 10954327-10957327 |
| C19orf33 | Chr19: 43485143-43488143 | C19orf33, Chr19: 43485143-43488143 |
| CCDC114 | Chr19: 53497644-53500644 | CCDC114, Chr19: 53497644-53500644 |
| TMEM190 | Chr19: 60578515-60581515 | TMEM190, Chr19: 60578515-60581515 |
| TUSC5 | Chr17: 1128206-1131206 | TUSC5, Chr17: 1128206-1131206 |
| MASP2 | Chr1: 11028372-11031372 | MASP2, Chr1: 11028372-11031372 |
| ALOX12B | Chr17: 7930246-7933246 | ALOX12B, Chr17: 7930246-7933246 |
| LAG3 | Chr12: 6750430-6753430 | LAG3, Chr12: 6750430-6753430 |
| MGC45438 | Chr16: 5054612-5057612 | MGC45438, Chr16: 5054612-5057612 |
| KLK6 | Chr19: 56163241-56166241 | KLK6, Chr19: 56163241-56166241 |
| FIT1 | Chr14: 23669107-23672107 | FIT1, Chr14: 23669107-23672107 |
| AGXT | Chr2: 241455334-241458334 | AGXT, Chr2: 241455334-241458334 |
| PSMB11 | Chr14: 22579715-22582715 | PSMB11, Chr14: 22579715-22582715 |
| PRM3 | Chr16: 11273453-11276453 | PRM3, Chr16: 11273453-11276453 |
| TRPV1 | Chr17: 3445585-3448585 | TRPV1, Chr17: 3445585-3448585 |
| CPN2 | Chr3: 195551850-195554850 | CPN2, Chr3: 195551850-195554850 |
| ACOT7 | Chr1: 6366970-6369970 | ACOT7, Chr1: 6366970-6369970 |
| PSPN | Chr19: 6325360-6328360 | PSPN, Chr19: 6325360-6328360 |
| GRM4 | Chr6: 34207921-34210921 | GRM4, Chr6: 34207921-34210921 |
| APLNR | Chr11: 56759989-56762989 | APLNR, Chr11: 56759989-56762989 |
| HOXB1 | Chr17: 43961771-43964771 | HOXB1, Chr17: 43961771-43964771 |
| MYLK2 | Chr20: 29869338-29872338 | MYLK2, Chr20: 29869338-29872338 |
| DDI1 | Chr11: 103411017-103414017 | DDI1, Chr11: 103411017-103414017 |
| ASB16 | Chr17: 39602099-39605099 | ASB16, Chr17: 39602099-39605099 |
| TMEM189- | Chr20: 48178089-48181089 | TMEM189-UBE2V1, Chr20: 48178089-48181089 |
| UBE2V1 | | |
| CAPG | Chr2: 85489687-85492687 | CAPG, Chr2: 85489687-85492687 |
| CETP | Chr16: 55551835-55554835 | CETP, Chr16: 55551835-55554835 |
| MAGEB10 | Chr23: 27734545-27737545 | MAGEB10, Chr23: 27734545-27737545 |
| KRT75 | Chr12: 51112877-51115877 | KRT75, Chr12: 51112877-51115877 |
| LTA | Chr6: 31646571-31649571 | LTA, Chr6: 31646571-31649571 |
| POU5F2 | Chr5: 93101565-93104565 | POU5F2, Chr5: 93101565-93104565 |
| ZNF556 | Chr19: 2816832-2819832 | ZNF556, Chr19: 2816832-2819832 |
| CCDC129 | Chr7: 31522002-31525002 | CCDC129, Chr7: 31522002-31525002 |
| ADCY7 | Chr16: 48877823-48880823 | ADCY7, Chr16: 48877823-48880823 |
| GAS2L2 | Chr17: 31102510-31105510 | GAS2L2, Chr17: 31102510-31105510 |
| KRT32 | Chr17: 36875664-36878664 | KRT32, Chr17: 36875664-36878664 |
| TUBA3C | Chr13: 18652436-18655436 | TUBA3C, Chr13: 18652436-18655436 |
| SOAT2 | Chr12: 51782040-51785040 | SOAT2, Chr12: 51782040-51785040 |
| KRTAP9-8 | Chr17: 36646295-36649295 | KRTAP9-8, Chr17: 36646295-36649295 |
| UGT1A5 | Chr2: 234284876-234287876 | UGT1A5, Chr2: 234284876-234287876 |
| MORC1 | Chr3: 110318158-110321158 | MORC1, Chr3: 110318158-110321158 |
| RGS3 | Chr9: 115302027-115305027 | RGS3, Chr9: 115302027-115305027 |
| CALD1 | Chr7: 134225198-134228198 | CALD1, Chr7: 134225198-134228198 |
| DTX2 | Chr7: 75946262-75949262 | DTX2, Chr7: 75946262-75949262 |
| CRISP2 | Chr6: 49787715-49790715 | CRISP2, Chr6: 49787715-49790715 |
| REM2 | Chr14: 22420771-22423771 | REM2, Chr14: 22420771-22423771 |
| RGR | Chr10: 85993288-85996288 | RGR, Chr10: 85993288-85996288 |
| C7orf34 | Chr7: 142345224-142348224 | C7orf34, Chr7: 142345224-142348224 |
| PTPDC1 | Chr9: 95885066-95888066 | PTPDC1, Chr9: 95885066-95888066 |
| KCNK16 | Chr6: 39396794-39399794 | KCNK16, Chr6: 39396794-39399794 |
| BPIL3 | Chr20: 31081614-31084614 | BPIL3, Chr20: 31081614-31084614 |
| PDE11A | Chr2: 178679812-178682812 | PDE11A, Chr2: 178679812-178682812 |
| C2orf61 | Chr2: 47234430-47237430 | C2orf61, Chr2: 47234430-47237430 |
| OOEP | Chr6: 74134736-74137736 | OOEP, Chr6: 74134736-74137736 |
| UGT1A4 | Chr2: 234290676-234293676 | UGT1A4, Chr2: 234290676-234293676 |
| SPATA16 | Chr3: 174340226-174343226 | SPATA16, Chr3: 174340226-174343226 |
| TEX12 | Chr11: 111541804-111544804 | TEX12, Chr11: 111541804-111544804 |
| APOBEC3H | Chr22: 37821735-37824735 | APOBEC3H, Chr22: 37821735-37824735 |
| KRTAP9-2 | Chr17: 36634925-36637925 | KRTAP9-2, Chr17: 36634925-36637925 |
| WISP2 | Chr20: 42775798-42778798 | WISP2, Chr20: 42775798-42778798 |
| SPDYC | Chr11: 64692782-64695782 | SPDYC, Chr11: 64692782-64695782 |
| SLC12A3 | Chr16: 55455119-55458119 | SLC12A3, Chr16: 55455119-55458119 |
| KIF4B | Chr5: 154371952-154374952 | KIF4B, Chr5: 154371952-154374952 |
| SERPINB5 | Chr18: 59293623-59296623 | SERPINB5, Chr18: 59293623-59296623 |
| MCCD1 | Chr6: 31603217-31606217 | MCCD1, Chr6: 31603217-31606217 |
| TMEM213 | Chr7: 138131778-138134778 | TMEM213, Chr7: 138131778-138134778 |
| LAMB3 | Chr1: 207890943-207893943 | LAMB3, Chr1: 207890943-207893943 |
| KRT9 | Chr17: 36980336-36983336 | KRT9, Chr17: 36980336-36983336 |
| MAGEB1 | Chr23: 30173691-30176691 | MAGEB1, Chr23: 30173691-30176691 |
| KIF2B | Chr17: 49253737-49256737 | KIF2B, Chr17: 49253737-49256737 |
| LOC100131439 | Chr17: 70098465-70101465 | LOC100131439, Chr17: 70098465-70101465 |
| EXTL1 | Chr1: 26219357-26222357 | EXTL1, Chr1: 26219357-26222357 |
| CYP11B2 | Chr8: 143994761-143997761 | CYP11B2, Chr8: 143994761-143997761 |
| GUCA2B | Chr1: 42390178-42393178 | GUCA2B, Chr1: 42390178-42393178 |
| MYOG | Chr1: 201320500-201323500 | MYOG, Chr1: 201320500-201323500 |
| LOC390667 | Chr16: 1476969-1479969 | LOC390667, Chr16: 1476969-1479969 |
| IL17C | Chr16: 87231001-87234001 | IL17C, Chr16: 87231001-87234001 |
| GGT6 | Chr17: 4409125-4412125 | GGT6, Chr17: 4409125-4412125 |
| TICAM1 | Chr19: 4767951-4770951 | TICAM1, Chr19: 4767951-4770951 |
| IGLL3 | Chr22: 24042387-24045387 | IGLL3, Chr22: 24042387-24045387 |
| ACY3 | Chr11: 67173206-67176206 | ACY3, Chr11: 67173206-67176206 |
| PROZ | Chr13: 112859468-112862468 | PROZ, Chr13: 112859468-112862468 |
| TINAGL1 | Chr1: 31813246-31816246 | TINAGL1, Chr1: 31813246-31816246 |
| DLGAP3 | Chr1: 35142071-35145071 | DLGAP3, Chr1: 35142071-35145071 |
| INPP5B | Chr1: 38183816-38186816 | INPP5B, Chr1: 38183816-38186816 |
| SHROOM1 | Chr5: 132188401-132191401 | SHROOM1, Chr5: 132188401-132191401 |
| CENTD2 | Chr11: 72100344-72103344 | CENTD2, Chr11: 72100344-72103344 |
| FBLIM1 | Chr1: 15962080-15965080 | FBLIM1, Chr1: 15962080-15965080 |
| TESSP2 | Chr3: 46849089-46852089 | TESSP2, Chr3: 46849089-46852089 |
| GPR56 | Chr16: 56209958-56212958 | GPR56, Chr16: 56209958-56212958 |
| KRTAP10-11 | Chr21: 44889258-44892258 | KRTAP10-11, Chr21: 44889258-44892258 |
| KRTAP12-2 | Chr21: 44909772-44912772 | KRTAP12-2, Chr21: 44909772-44912772 |
| KRTAP12-1 | Chr21: 44925006-44928006 | KRTAP12-1, Chr21: 44925006-44928006 |
| LGLL338 | Chr9: 35894688-35897688 | LGLL338, Chr9: 35894688-35897688 |
| CENTD2 | Chr11: 72114321-72117321 | CENTD2, Chr11: 72114321-72117321 |
| ANKRD33 | Chr12: 50566559-50569559 | ANKRD33, Chr12: 50566559-50569559 |
| TMEM119 | Chr12: 107514523-107517523 | TMEM119, Chr12: 107514523-107517523 |
| ZBTB7C | Chr18: 43819992-43822992 | ZBTB7C, Chr18: 43819992-43822992 |
| AGRP | Chr16: 66073305-66076305 | AGRP, Chr16: 66073305-66076305 |
| GUCA2A | Chr1: 42401482-42404482 | GUCA2A, Chr1: 42401482-42404482 |
| LAPTM5 | Chr1: 31001754-31004754 | LAPTM5, Chr1: 31001754-31004754 |
| FGD3 | Chr9: 94775038-94778038 | FGD3, Chr9: 94775038-94778038 |
| GPR182 | Chr12: 55673121-55676121 | GPR182, Chr12: 55673121-55676121 |
| MYBPH | Chr1: 201410065-201413065 | MYBPH, Chr1: 201410065-201413065 |
| UBASH3A | Chr21: 42695587-42698587 | UBASH3A, Chr21: 42695587-42698587 |
| NCF4 | Chr22: 35585475-35588475 | NCF4, Chr22: 35585475-35588475 |
| SDK1 | Chr7: 4134342-4137342 | SDK1, Chr7: 4134342-4137342 |
| AQP6 | Chr12: 48651386-48654386 | AQP6, Chr12: 48651386-48654386 |
| SLC4A1 | Chr17: 39699528-39702528 | SLC4A1, Chr17: 39699528-39702528 |
| GJB1 | Chr23: 70358280-70361280 | GJB1, Chr23: 70358280-70361280 |
| CCDC81 | Chr11: 85761925-85764925 | CCDC81, Chr11: 85761925-85764925 |
| OTOF | Chr2: 26633570-26636570 | OTOF, Chr2: 26633570-26636570 |
| KRTAP5-10 | Chr11: 70952756-70955756 | KRTAP5-10, Chr11: 70952756-70955756 |
| GPR179 | Chr17: 33751719-33754719 | GPR179, Chr17: 33751719-33754719 |
| FAM9C | Chr23: 12971221-12974221 | FAM9C, Chr23: 12971221-12974221 |
| KRTAP4-5 | Chr17: 36558080-36561080 | KRTAP4-5, Chr17: 36558080-36561080 |
| GPR31 | Chr6: 167489809-167492809 | GPR31, Chr6: 167489809-167492809 |
| FGF6 | Chr12: 4423541-4426541 | FGF6, Chr12: 4423541-4426541 |
| AOC2 | Chr17: 38248634-38251634 | AOC2, Chr17: 38248634-38251634 |
| RFX4 | Chr12: 105517579-105520579 | RFX4, Chr12: 105517579-105520579 |
| TUBB8 | Chr10: 83678-86678 | TUBB8, Chr10: 83678-86678 |
| PRM2 | Chr16: 11276338-11279338 | PRM2, Chr16: 11276338-11279338 |
| EGFLAM | Chr5: 38479892-38482892 | EGFLAM, Chr5: 38479892-38482892 |
| KRTAP5-11 | Chr11: 70970069-70973069 | KRTAP5-11, Chr11: 70970069-70973069 |
| CSNK1A1L | Chr13: 36576301-36579301 | CSNK1A1L, Chr13: 36576301-36579301 |
| KRTAP12-4 | Chr21: 44897504-44900504 | KRTAP12-4, Chr21: 44897504-44900504 |
| FAM154A | Chr9: 19021684-19024684 | FAM154A, Chr9: 19021684-19024684 |
| NPHS1 | Chr19: 41033079-41036079 | NPHS1, Chr19: 41033079-41036079 |
| RNF17 | Chr13: 24234800-24237800 | RNF17, Chr13: 24234800-24237800 |
| TCL1B | Chr14: 95221015-95224015 | TCL1B, Chr14: 95221015-95224015 |
| TNFRSF13B | Chr17: 16814627-16817627 | TNFRSF13B, Chr17: 16814627-16817627 |
| ZP3 | Chr7: 75863276-75866276 | ZP3, Chr7: 75863276-75866276 |
| FASLG | Chr1: 170893307-170896307 | FASLG, Chr1: 170893307-170896307 |
| CAMK2A | Chr5: 149648029-149651029 | CAMK2A, Chr5: 149648029-149651029 |
| KRTAP5-6 | Chr11: 1673500-1676500 | KRTAP5-6, Chr11: 1673500-1676500 |
| LRIT1 | Chr10: 85989697-85992697 | LRIT1, Chr10: 85989697-85992697 |
| KRT31 | Chr17: 36805870-36808870 | KRT31, Chr17: 36805870-36808870 |
| VAV1 | Chr19: 6722221-6725221 | VAV1, Chr19: 6722221-6725221 |
| KRTAP9-4 | Chr17: 36657964-36660964 | KRTAP9-4, Chr17: 36657964-36660964 |
| KRT33A | Chr17: 36759082-36762082 | KRT33A, Chr17: 36759082-36762082 |
| GPBAR1 | Chr2: 218832482-218835482 | GPBAR1, Chr2: 218832482-218835482 |
| NEU2 | Chr2: 233604125-233607125 | NEU2, Chr2: 233604125-233607125 |
| PXDNL | Chr8: 52883058-52886058 | PXDNL, Chr8: 52883058-52886058 |
| ITGAE | Chr17: 3649786-3652786 | ITGAE, Chr17: 3649786-3652786 |
| ARHGEF10L | Chr1: 17778134-17781134 | ARHGEF10L, Chr1: 17778134-17781134 |
| CTRC | Chr1: 15636024-15639024 | CTRC, Chr1: 15636024-15639024 |
| KRT33B | Chr17: 36778073-36781073 | KRT33B, Chr17: 36778073-36781073 |
| PRSS8 | Chr16: 31053152-31056152 | PRSS8, Chr16: 31053152-31056152 |
| KRT86 | Chr12: 50980415-50983415 | KRT86, Chr12: 50980415-50983415 |
| MGC33407 | Chr19: 8668672-8671672 | MGC33407, Chr19: 8668672-8671672 |
| CYP11B1 | Chr8: 143956738-143959738 | CYP11B1, Chr8: 143956738-143959738 |
| TET3 | Chr2: 74125457-74128457 | TET3, Chr2: 74125457-74128457 |
| C6orf221 | Chr6: 74127620-74130620 | C6orf221, Chr6: 74127620-74130620 |
| KRTAP5-1 | Chr11: 1561589-1564589 | KRTAP5-1, Chr11: 1561589-1564589 |
| SPI1 | Chr11: 47355203-47358203 | SPI1, Chr11: 47355203-47358203 |
| BAGE5 | Chr21: 10119296-10122296 | BAGE5, Chr21: 10119296-10122296 |
| FAM163B | Chr9: 135433666-135436666 | FAM163B, Chr9: 135433666-135436666 |
| KRTAP10-7 | Chr21: 44843424-44846424 | KRTAP10-7, Chr21: 44843424-44846424 |
| ZSCAN10 | Chr16: 3081362-3084362 | ZSCAN10, Chr16: 3081362-3084362 |
| DMKN | Chr19: 40683153-40686153 | DMKN, Chr19: 40683153-40686153 |
| C11orf16 | Chr11: 8909629-8912629 | C11orf16, Chr11: 8909629-8912629 |
| RGS3 | Chr9: 115394520-115397520 | RGS3, Chr9: 115394520-115397520 |
| C3orf22 | Chr3: 127758948-127761948 | C3orf22, Chr3: 127758948-127761948 |
| ANGPT4 | Chr20: 843460-846460 | ANGPT4, Chr20: 843460-846460 |
| C1orf177 | Chr1: 55042823-55045823 | C1orf177, Chr1: 55042823-55045823 |
| SEMA3B | Chr3: 50278543-50281543 | SEMA3B, Chr3: 50278543-50281543 |
| CYP21A2 | Chr6: 32112560-32115560 | CYP21A2, Chr6: 32112560-32115560 |
| C6orf25 | Chr6: 31797639-31800639 | C6orf25, Chr6: 31797639-31800639 |
| GALP | Chr19: 61377700-61380700 | GALP, Chr19: 61377700-61380700 |
| RNF186 | Chr1: 20012858-20015858 | RNF186, Chr1: 20012858-20015858 |
| AMAC1 | Chr17: 30543990-30546990 | AMAC1, Chr17: 30543990-30546990 |
| KRTAP4-4 | Chr17: 36569009-36572009 | KRTAP4-4, Chr17: 36569009-36572009 |
| KLHL33 | Chr14: 19966934-19969934 | KLHL33, Chr14: 19966934-19969934 |
| PRKAG2 | Chr7: 151141390-151144390 | PRKAG2, Chr7: 151141390-151144390 |
| AVPR1B | Chr1: 204389405-204392405 | AVPR1B, Chr1: 204389405-204392405 |
| BFSP2 | Chr3: 134599979-134602979 | BFSP2, Chr3: 134599979-134602979 |
| KRTAP5-9 | Chr11: 70935613-70938613 | KRTAP5-9, Chr11: 70935613-70938613 |
| KRTAP4-12 | Chr17: 36532445-36535445 | KRTAP4-12, Chr17: 36532445-36535445 |
| HIPK4 | Chr19: 45586434-45589434 | HIPK4, Chr19: 45586434-45589434 |
| RGS3 | Chr9: 115381260-115384260 | RGS3, Chr9: 115381260-115384260 |
| CACNA2D4 | Chr12: 1896631-1899631 | CACNA2D4, Chr12: 1896631-1899631 |
| HAPLN4 | Chr19: 19233060-19236060 | HAPLN4, Chr19: 19233060-19236060 |
| FAM90A1 | Chr12: 8269981-8272981 | FAM90A1, Chr12: 8269981-8272981 |
| PDE4DIP | Chr1: 143704879-143707879 | PDE4DIP, Chr1: 143704879-143707879 |
| ELF5 | Chr11: 34490406-34493406 | ELF5, Chr11: 34490406-34493406 |
| FOLR3 | Chr11: 71522918-71525918 | FOLR3, Chr11: 71522918-71525918 |
| NUDT12 | Chr5: 102924889-102927889 | NUDT12, Chr5: 102924889-102927889 |
| APOC4 | Chr19: 50135834-50138834 | APOC4, Chr19: 50135834-50138834 |
| FAM3D | Chr3: 58626101-58629101 | FAM3D, Chr3: 58626101-58629101 |
| KRTAP3-2 | Chr17: 36408164-36411164 | KRTAP3-2, Chr17: 36408164-36411164 |
| ABRA | Chr8: 107850148-107853148 | ABRA, Chr8: 107850148-107853148 |
| GNLY | Chr2: 85773424-85776424 | GNLY, Chr2: 85773424-85776424 |
| KIAA1462 | Chr10: 30375306-30378306 | KIAA1462, Chr10: 30375306-30378306 |
| SLC1A6 | Chr19: 14943230-14946230 | SLC1A6, Chr19: 14943230-14946230 |
| SLC22A1 | Chr6: 160461352-160464352 | SLC22A1, Chr6: 160461352-160464352 |
| KRT34 | Chr17: 36790662-36793662 | KRT34, Chr17: 36790662-36793662 |
| GJB4 | Chr1: 34997864-35000864 | GJB4, Chr1: 34997864-35000864 |
| RCAN2 | Chr6: 46399990-46402990 | RCAN2, Chr6: 46399990-46402990 |
| MRVI1 | Chr11: 10628924-10631924 | MRVI1, Chr11: 10628924-10631924 |
| KRTAP5-7 | Chr11: 70914460-70917460 | KRTAP5-7, Chr11: 70914460-70917460 |
| KRT37 | Chr17: 36832848-36835848 | KRT37, Chr17: 36832848-36835848 |
| MRVI1 | Chr11: 10670611-10673611 | MRVI1, Chr11: 10670611-10673611 |
| ARSG | Chr17: 63765417-63768417 | ARSG, Chr17: 63765417-63768417 |
| OR10H5 | Chr19: 15764358-15767358 | OR10H5, Chr19: 15764358-15767358 |
| C14orf49 | Chr14: 95010426-95013426 | C14orf49, Chr14: 95010426-95013426 |
| KRTAP2-4 | Chr17: 36474157-36477157 | KRTAP2-4, Chr17: 36474157-36477157 |
| TNP2 | Chr16: 11269161-11272161 | TNP2, Chr16: 11269161-11272161 |
| RLN3 | Chr19: 13998516-14001516 | RLN3, Chr19: 13998516-14001516 |
| MCART2 | Chr18: 27593341-27596341 | MCART2, Chr18: 27593341-27596341 |
| CXorf36 | Chr23: 44943590-44946590 | CXorf36, Chr23: 44943590-44946590 |
| RGPD1 | Chr2: 86992916-86995916 | RGPD1, Chr2: 86992916-86995916 |
| RGPD1 | Chr2: 87904930-87907930 | RGPD1, Chr2: 87904930-87907930 |
| KLK11 | Chr19: 56221197-56224197 | KLK11, Chr19: 56221197-56224197 |
| SLC22A6 | Chr11: 62507545-62510545 | SLC22A6, Chr11: 62507545-62510545 |
| SYTL1 | Chr1: 27539599-27542599 | SYTL1, Chr1: 27539599-27542599 |
| PVRIG | Chr7: 99653306-99656306 | PVRIG, Chr7: 99653306-99656306 |
| PYGM | Chr11: 64283263-64286263 | PYGM, Chr11: 64283263-64286263 |
| CCDC78 | Chr16: 714960-717960 | CCDC78, Chr16: 714960-717960 |
| MYLK3 | Chr16: 45338222-45341222 | MYLK3, Chr16: 45338222-45341222 |
| C17orf74 | Chr17: 7268158-7271158 | C17orf74, Chr17: 7268158-7271158 |
| CSF3 | Chr17: 35423713-35426713 | CSF3, Chr17: 35423713-35426713 |
| LRRC25 | Chr19: 18367915-18370915 | LRRC25, Chr19: 18367915-18370915 |
| SIGLEC1 | Chr20: 3634275-3637275 | SIGLEC1, Chr20: 3634275-3637275 |
| BAGE | Chr21: 10119308-10122308 | BAGE, Chr21: 10119308-10122308 |
| ABCC10 | Chr6: 43505966-43508966 | ABCC10, Chr6: 43505966-43508966 |
| ETV3L | Chr1: 155334724-155337724 | ETV3L, Chr1: 155334724-155337724 |
| ACRC | Chr23: 70713485-70716485 | ACRC, Chr23: 70713485-70716485 |
| CXCR5 | Chr11: 118267810-118270810 | CXCR5, Chr11: 118267810-118270810 |
| SPN | Chr16: 29580580-29583580 | SPN, Chr16: 29580580-29583580 |
| RXFP4 | Chr1: 154176603-154179603 | RXFP4, Chr1: 154176603-154179603 |
| FGFR4 | Chr5: 176447656-176450656 | FGFR4, Chr5: 176447656-176450656 |
| NRTN | Chr19: 5773317-5776317 | NRTN, Chr19: 5773317-5776317 |
| MLC1 | Chr22: 48864985-48867985 | MLC1, Chr22: 48864985-48867985 |
| MATN1 | Chr1: 30967974-30970974 | MATN1, Chr1: 30967974-30970974 |
| TTC24 | Chr1: 154817145-154820145 | TTC24, Chr1: 154817145-154820145 |
| MYEOV | Chr11: 68816697-68819697 | MYEOV, Chr11: 68816697-68819697 |
| PDZD2 | Chr5: 31833287-31836287 | PDZD2, Chr5: 31833287-31836287 |
| SLC2A9 | Chr4: 9630712-9633712 | SLC2A9, Chr4: 9630712-9633712 |
| F2 | Chr11: 46695818-46698818 | F2, Chr11: 46695818-46698818 |
| TNFSF12 | Chr17: 7391598-7394598 | TNFSF12, Chr17: 7391598-7394598 |
| KRTAP5-3 | Chr11: 1584769-1587769 | KRTAP5-3, Chr11: 1584769-1587769 |
| TNFSF12-TNFSF13 | Chr17: 7391639-7394639 | TNFSF12-TNFSF13, Chr17: 7391639-7394639 |
| CYP4F8 | Chr19: 15585528-15588528 | CYP4F8, Chr19: 15585528-15588528 |
| HLA-DOA | Chr6: 33083867-33086867 | HLA-DOA, Chr6: 33083867-33086867 |
| ALDH3B2 | Chr11: 67197178-67200178 | ALDH3B2, Chr11: 67197178-67200178 |
| KRTAP4-11 | Chr17: 36526638-36529638 | KRTAP4-11, Chr17: 36526638-36529638 |
| KRT85 | Chr12: 51046076-51049076 | KRT85, Chr12: 51046076-51049076 |
| NPFFR2 | Chr4: 73114884-73117884 | NPFFR2, Chr4: 73114884-73117884 |
| MCHR1 | Chr22: 39403627-39406627 | MCHR1, Chr22: 39403627-39406627 |
| CSF1R | Chr5: 149471628-149474628 | CSF1R, Chr5: 149471628-149474628 |
| AMBP | Chr9: 115879073-115882073 | AMBP, Chr9: 115879073-115882073 |
| KRT82 | Chr12: 51084943-51087943 | KRT82, Chr12: 51084943-51087943 |
| VHLL | Chr1: 154534214-154537214 | VHLL, Chr1: 154534214-154537214 |
| TRIML1 | Chr4: 189296091-189299091 | TRIML1, Chr4: 189296091-189299091 |
| ACOT7 | Chr1: 6341851-6344851 | ACOT7, Chr1: 6341851-6344851 |
| OR10C1 | Chr6: 29514271-29517271 | OR10C1, Chr6: 29514271-29517271 |
| FGD2 | Chr6: 37079900-37082900 | FGD2, Chr6: 37079900-37082900 |
| PDE6C | Chr10: 95360834-95363834 | PDE6C, Chr10: 95360834-95363834 |
| ABCG1 | Chr21: 42491490-42494490 | ABCG1, Chr21: 42491490-42494490 |
| SLC38A11 | Chr2: 165518781-165521781 | SLC38A11, Chr2: 165518781-165521781 |
| KRT71 | Chr12: 51231670-51234670 | KRT71, Chr12: 51231670-51234670 |
| MSGN1 | Chr2: 17859766-17862766 | MSGN1, Chr2: 17859766-17862766 |
| ATP6V0A4 | Chr7: 138131981-138134981 | ATP6V0A4, Chr7: 138131981-138134981 |
| PM20D1 | Chr1: 204084368-204087368 | PM20D1, Chr1: 204084368-204087368 |
| ISCA1L | Chr5: 62107426-62110426 | ISCA1L, Chr5: 62107426-62110426 |
| OR10H2 | Chr19: 15698333-15701333 | OR10H2, Chr19: 15698333-15701333 |
| TSPY1 | Chr24: 9913063-9916063 | TSPY1, Chr24: 9913063-9916063 |
| OR1Q1 | Chr9: 124415337-124418337 | OR1Q1, Chr9: 124415337-124418337 |
| CTBP2 | Chr10: 126704943-126707943 | CTBP2, Chr10: 126704943-126707943 |
| CEBPE | Chr14: 22656814-22659814 | CEBPE, Chr14: 22656814-22659814 |
| YIF1B | Chr19: 43490849-43493849 | YIF1B, Chr19: 43490849-43493849 |
| SDCBP2 | Chr20: 1240803-1243803 | SDCBP2, Chr20: 1240803-1243803 |
| KRTAP10-5 | Chr21: 44823409-44826409 | KRTAP10-5, Chr21: 44823409-44826409 |
| ITGB2 | Chr21: 45163893-45166893 | ITGB2, Chr21: 45163893-45166893 |
| PARVG | Chr22: 42906689-42909689 | PARVG, Chr22: 42906689-42909689 |
| PLA2G4D | Chr15: 40172544-40175544 | PLA2G4D, Chr15: 40172544-40175544 |
| ATP4A | Chr19: 40744900-40747900 | ATP4A, Chr19: 40744900-40747900 |
| EPX | Chr17: 53623620-53626620 | EPX, Chr17: 53623620-53626620 |
| CYB5R3 | Chr22: 41371439-41374439 | CYB5R3, Chr22: 41371439-41374439 |
| SCARF1 | Chr17: 1494291-1497291 | SCARF1, Chr17: 1494291-1497291 |
| CTCFL | Chr20: 55532060-55535060 | CTCFL, Chr20: 55532060-55535060 |
| SIT1 | Chr9: 35639447-35642447 | SIT1, Chr9: 35639447-35642447 |
| KCNN3 | Chr1: 153107878-153110878 | KCNN3, Chr1: 153107878-153110878 |
| RCVRN | Chr17: 9747909-9750909 | RCVRN, Chr17: 9747909-9750909 |
| RAPGEF3 | Chr12: 46436948-46439948 | RAPGEF3, Chr12: 46436948-46439948 |
| OR10Q1 | Chr11: 57751423-57754423 | OR10Q1, Chr11: 57751423-57754423 |
| FIBIN | Chr11: 26970703-26973703 | FIBIN, Chr11: 26970703-26973703 |
| HAL | Chr12: 94912702-94915702 | HAL, Chr12: 94912702-94915702 |
| KCNK7 | Chr11: 65118543-65121543 | KCNK7, Chr11: 65118543-65121543 |
| KRT5 | Chr12: 51199010-51202010 | KRT5, Chr12: 51199010-51202010 |
| OR2W3 | Chr1: 246124011-246127011 | OR2W3, Chr1: 246124011-246127011 |
| ENPEP | Chr4: 111615177-111618177 | ENPEP, Chr4: 111615177-111618177 |
| NANOS2 | Chr19: 51108376-51111376 | NANOS2, Chr19: 51108376-51111376 |
| KRTAP1-5 | Chr17: 36435480-36438480 | KRTAP1-5, Chr17: 36435480-36438480 |
| KRTAP1-3 | Chr17: 36443133-36446133 | KRTAP1-3, Chr17: 36443133-36446133 |
| ABCG1 | Chr21: 42491367-42494367 | ABCG1, Chr21: 42491367-42494367 |
| SH2D1A | Chr23: 123306328-123309328 | SH2D1A, Chr23: 123306328-123309328 |
| ZP3 | Chr7: 75890707-75893707 | ZP3, Chr7: 75890707-75893707 |
| KLK9 | Chr19: 56203202-56206202 | KLK9, Chr19: 56203202-56206202 |
| TUBB4Q | Chr4: 191141518-191144518 | TUBB4Q, Chr4: 191141518-191144518 |
| MFAP4 | Chr17: 19229586-19232586 | MFAP4, Chr17: 19229586-19232586 |
| MORN3 | Chr12: 120590443-120593443 | MORN3, Chr12: 120590443-120593443 |
| GAGE12J | Chr23: 49063959-49066959 | GAGE12J, Chr23: 49063959-49066959 |
| PRAM1 | Chr19: 8471995-8474995 | PRAM1, Chr19: 8471995-8474995 |
| KRT83 | Chr12: 50999949-51002949 | KRT83, Chr12: 50999949-51002949 |
| MEFV | Chr16: 3245128-3248128 | MEFV, Chr16: 3245128-3248128 |
| AGT | Chr1: 228915459-228918459 | AGT, Chr1: 228915459-228918459 |
| HYAL1 | Chr3: 50314508-50317508 | HYAL1, Chr3: 50314508-50317508 |
| SEMA6B | Chr19: 4508003-4511003 | SEMA6B, Chr19: 4508003-4511003 |
| GGT5 | Chr22: 22969610-22972610 | GGT5, Chr22: 22969610-22972610 |
| WBSCR27 | Chr7: 72893291-72896291 | WBSCR27, Chr7: 72893291-72896291 |
| C16orf54 | Chr16: 29663341-29666341 | C16orf54, Chr16: 29663341-29666341 |
| RAPGEF3 | Chr12: 46437011-46440011 | RAPGEF3, Chr12: 46437011-46440011 |
| C1R | Chr12: 7134684-7137684 | C1R, Chr12: 7134684-7137684 |
| PROP1 | Chr5: 177354349-177357349 | PROP1, Chr5: 177354349-177357349 |
| GMEB2 | Chr20: 61720173-61723173 | GMEB2, Chr20: 61720173-61723173 |
| S100P | Chr4: 6744966-6747966 | S100P, Chr4: 6744966-6747966 |
| SLC39A5 | Chr12: 54909393-54912393 | SLC39A5, Chr12: 54909393-54912393 |
| CFP | Chr23: 47373148-47376148 | CFP, Chr23: 47373148-47376148 |
| EPS8L1 | Chr19: 60277532-60280532 | EPS8L1, Chr19: 60277532-60280532 |
| PRKAG3 | Chr2: 219403256-219406256 | PRKAG3, Chr2: 219403256-219406256 |
| AOC3 | Chr17: 38255226-38258226 | AOC3, Chr17: 38255226-38258226 |
| CYP4F11 | Chr19: 15905176-15908176 | CYP4F11, Chr19: 15905176-15908176 |
| KRTAP2-1 | Chr17: 36455594-36458594 | KRTAP2-1, Chr17: 36455594-36458594 |
| SIPA1L1 | Chr14: 71064294-71067294 | SIPA1L1, Chr14: 71064294-71067294 |
| P2RY4 | Chr23: 69394879-69397879 | P2RY4, Chr23: 69394879-69397879 |
| FGD5 | Chr3: 14833972-14836972 | FGD5, Chr3: 14833972-14836972 |
| NKG7 | Chr19: 56566272-56569272 | NKG7, Chr19: 56566272-56569272 |
| GPR172B | Chr17: 4877951-4880951 | GPR172B, Chr17: 4877951-4880951 |
| BCL2L14 | Chr12: 12114168-12117168 | BCL2L14, Chr12: 12114168-12117168 |
| SPN | Chr16: 29580300-29583300 | SPN, Chr16: 29580300-29583300 |
| C20orf141 | Chr20: 2742156-2745156 | C20orf141, Chr20: 2742156-2745156 |
| C22orf15 | Chr22: 22433707-22436707 | C22orf15, Chr22: 22433707-22436707 |
| NFKBIL1 | Chr6: 31621850-31624850 | NFKBIL1, Chr6: 31621850-31624850 |
| MKRN3 | Chr15: 21360046-21363046 | MKRN3, Chr15: 21360046-21363046 |
| GGTLC2 | Chr22: 21317279-21320279 | GGTLC2, Chr22: 21317279-21320279 |
| PDE11A | Chr2: 178644228-178647228 | PDE11A, Chr2: 178644228-178647228 |
| DPEP1 | Chr16: 88205716-88208716 | DPEP1, Chr16: 88205716-88208716 |
| NMUR2 | Chr5: 151763533-151766533 | NMUR2, Chr5: 151763533-151766533 |
| CCT8L2 | Chr22: 15452200-15455200 | CCT8L2, Chr22: 15452200-15455200 |
| DDR1 | Chr6: 30962943-30965943 | DDR1, Chr6: 30962943-30965943 |
| KNG1 | Chr3: 187916313-187919313 | KNG1, Chr3: 187916313-187919313 |
| MLL2 | Chr12: 47733874-47736874 | MLL2, Chr12: 47733874-47736874 |
| MRGPRD | Chr11: 68503531-68506531 | MRGPRD, Chr11: 68503531-68506531 |
| ADCY6 | Chr12: 47462644-47465644 | ADCY6, Chr12: 47462644-47465644 |
| ProSAPiP1 | Chr20: 3095707-3098707 | ProSAPiP1, Chr20: 3095707-3098707 |
| COL6A1 | Chr21: 46224590-46227590 | COL6A1, Chr21: 46224590-46227590 |
| GHDC | Chr17: 37598239-37601239 | GHDC, Chr17: 37598239-37601239 |
| FFAR3 | Chr19: 40539841-40542841 | FFAR3, Chr19: 40539841-40542841 |
| KCNE4 | Chr2: 223623605-223626605 | KCNE4, Chr2: 223623605-223626605 |
| SLC4A9 | Chr5: 139718470-139721470 | SLC4A9, Chr5: 139718470-139721470 |
| DIRAS3 | Chr1: 68287548-68290548 | DIRAS3, Chr1: 68287548-68290548 |
| ZNF385C | Chr17: 37432733-37435733 | ZNF385C, Chr17: 37432733-37435733 |
| DUSP10 | Chr1: 219975925-219978925 | DUSP10, Chr1: 219975925-219978925 |
| MRGPRX1 | Chr11: 18911625-18914625 | MRGPRX1, Chr11: 18911625-18914625 |
| HTR1D | Chr1: 23392309-23395309 | HTR1D, Chr1: 23392309-23395309 |
| GAGE2A | Chr23: 49239561-49242561 | GAGE2A, Chr23: 49239561-49242561 |
| KLK11 | Chr19: 56221602-56224602 | KLK11, Chr19: 56221602-56224602 |
| PRODH2 | Chr19: 40994541-40997541 | PRODH2, Chr19: 40994541-40997541 |
| CD247 | Chr1: 165752971-165755971 | CD247, Chr1: 165752971-165755971 |
| SLC34A1 | Chr5: 176742540-176745540 | SLC34A1, Chr5: 176742540-176745540 |
| BPIL1 | Chr20: 31057567-31060567 | BPIL1, Chr20: 31057567-31060567 |
| CDK5R1 | Chr17: 27836717-27839717 | CDK5R1, Chr17: 27836717-27839717 |
| FRMD4A | Chr10: 14411372-14414372 | FRMD4A, Chr10: 14411372-14414372 |
| C14orf115 | Chr14: 73883418-73886418 | C14orf115, Chr14: 73883418-73886418 |
| APOC3 | Chr11: 116204333-116207333 | APOC3, Chr11: 116204333-116207333 |
| SATL1 | Chr23: 84248602-84251602 | SATL1, Chr23: 84248602-84251602 |
| ADORA1 | Chr1: 201361958-201364958 | ADORA1, Chr1: 201361958-201364958 |
| RNF183 | Chr9: 115099641-115102641 | RNF183, Chr9: 115099641-115102641 |
| CTRL | Chr16: 66521766-66524766 | CTRL, Chr16: 66521766-66524766 |
| CNFN | Chr19: 47584784-47587784 | CNFN, Chr19: 47584784-47587784 |
| TSSK1B | Chr5: 112797065-112800065 | TSSK1B, Chr5: 112797065-112800065 |
| NLRP1 | Chr17: 5427056-5430056 | NLRP1, Chr17: 5427056-5430056 |
| PTHR1 | Chr3: 46892739-46895739 | PTHR1, Chr3: 46892739-46895739 |
| KLC3 | Chr19: 50534337-50537337 | KLC3, Chr19: 50534337-50537337 |
| DPEP2 | Chr16: 66589357-66592357 | DPEP2, Chr16: 66589357-66592357 |
| PRSS36 | Chr16: 31067388-31070388 | PRSS36, Chr16: 31067388-31070388 |
| WFDC5 | Chr20: 43175717-43178717 | WFDC5, Chr20: 43175717-43178717 |
| C2orf27 | Chr2: 132195033-132198033 | C2orf27, Chr2: 132195033-132198033 |
| RAPGEF3 | Chr12: 46437452-46440452 | RAPGEF3, Chr12: 46437452-46440452 |
| EFCAB3 | Chr17: 57810145-57813145 | EFCAB3, Chr17: 57810145-57813145 |
| COL6A3 | Chr2: 237986089-237989089 | COL6A3, Chr2: 237986089-237989089 |
| IL13 | Chr5: 132020263-132023263 | IL13, Chr5: 132020263-132023263 |
| DUSP13 | Chr10: 76537476-76540476 | DUSP13, Chr10: 76537476-76540476 |
| APOA5 | Chr11: 116166294-116169294 | APOA5, Chr11: 116166294-116169294 |
| TAPBPL | Chr12: 6429937-6432937 | TAPBPL, Chr12: 6429937-6432937 |
| CYP1A2 | Chr15: 72826736-72829736 | CYP1A2, Chr15: 72826736-72829736 |
| CHRNE | Chr17: 4745648-4748648 | CHRNE, Chr17: 4745648-4748648 |
| RUNDC3A | Chr17: 39739989-39742989 | RUNDC3A, Chr17: 39739989-39742989 |
| SPATA20 | Chr17: 45978060-45981060 | SPATA20, Chr17: 45978060-45981060 |
| BCL2L2 | Chr14: 22844351-22847351 | BCL2L2, Chr14: 22844351-22847351 |
| IL28B | Chr19: 44425951-44428951 | IL28B, Chr19: 44425951-44428951 |
| KRT23 | Chr17: 36345862-36348862 | KRT23, Chr17: 36345862-36348862 |
| FSD1 | Chr19: 4254190-4257190 | FSD1, Chr19: 4254190-4257190 |
| TMEM115 | Chr3: 50370443-50373443 | TMEM115, Chr3: 50370443-50373443 |
| TRIM7 | Chr5: 180559036-180562036 | TRIM7, Chr5: 180559036-180562036 |
| ZNF516 | Chr18: 72302543-72305543 | ZNF516, Chr18: 72302543-72305543 |
| BCL2L14 | Chr12: 12113644-12116644 | BCL2L14, Chr12: 12113644-12116644 |
| GGT1 | Chr22: 23308217-23311217 | GGT1, Chr22: 23308217-23311217 |
| IL28A | Chr19: 44449496-44452496 | IL28A, Chr19: 44449496-44452496 |
| CENPM | Chr22: 40664669-40667669 | CENPM, Chr22: 40664669-40667669 |
| FLJ25404 | Chr16: 29940655-29943655 | FLJ25404, Chr16: 29940655-29943655 |
| CLDN20 | Chr6: 155625338-155628338 | CLDN20, Chr6: 155625338-155628338 |
| REM1 | Chr20: 29525265-29528265 | REM1, Chr20: 29525265-29528265 |
| IFI27 | Chr14: 93645331-93648331 | IFI27, Chr14: 93645331-93648331 |
| VWF | Chr12: 6102597-6105597 | VWF, Chr12: 6102597-6105597 |
| TFF1 | Chr21: 42658213-42661213 | TFF1, Chr21: 42658213-42661213 |
| USP21 | Chr1: 159394377-159397377 | USP21, Chr1: 159394377-159397377 |
| FAM71A | Chr1: 210862938-210865938 | FAM71A, Chr1: 210862938-210865938 |
| GP1BA | Chr17: 4774871-4777871 | GP1BA, Chr17: 4774871-4777871 |
| TSPAN18 | Chr11: 44836954-44839954 | TSPAN18, Chr11: 44836954-44839954 |
| C3orf36 | Chr3: 135129846-135132846 | C3orf36, Chr3: 135129846-135132846 |
| SLC2A7 | Chr1: 9007491-9010491 | SLC2A7, Chr1: 9007491-9010491 |
| PIK3R5 | Chr17: 8755059-8758059 | PIK3R5, Chr17: 8755059-8758059 |
| DHRS4 | Chr14: 23491304-23494304 | DHRS4, Chr14: 23491304-23494304 |
| IL4 | Chr5: 132035771-132038771 | IL4, Chr5: 132035771-132038771 |
| BZRPL1 | Chr6: 41116770-41119770 | BZRPL1, Chr6: 41116770-41119770 |
| CNGB1 | Chr16: 56561013-56564013 | CNGB1, Chr16: 56561013-56564013 |
| TEPP | Chr16: 56566339-56569339 | TEPP, Chr16: 56566339-56569339 |
| MYBPC3 | Chr11: 47329329-47332329 | MYBPC3, Chr11: 47329329-47332329 |
| MFSD5 | Chr12: 51930646-51933646 | MFSD5, Chr12: 51930646-51933646 |
| ZBTB46 | Chr20: 61905800-61908800 | ZBTB46, Chr20: 61905800-61908800 |
| CXCR3 | Chr23: 70753592-70756592 | CXCR3, Chr23: 70753592-70756592 |
| FAM123B | Chr23: 63340849-63343849 | FAM123B, Chr23: 63340849-63343849 |
| DNHD1 | Chr11: 6542410-6545410 | DNHD1, Chr11: 6542410-6545410 |
| IL1F8 | Chr2: 113525411-113528411 | IL1F8, Chr2: 113525411-113528411 |
| HYAL4 | Chr7: 123270958-123273958 | HYAL4, Chr7: 123270958-123273958 |
| FGR | Chr1: 27823838-27826838 | FGR, Chr1: 27823838-27826838 |
| IYD | Chr6: 150730220-150733220 | IYD, Chr6: 150730220-150733220 |
| ANKRD43 | Chr5: 132175431-132178431 | ANKRD43, Chr5: 132175431-132178431 |
| ZNF238 | Chr1: 242281704-242284704 | ZNF238, Chr1: 242281704-242284704 |
| FAM19A3 | Chr1: 113063211-113066211 | FAM19A3, Chr1: 113063211-113066211 |
| THOC5 | Chr22: 28278144-28281144 | THOC5, Chr22: 28278144-28281144 |
| DPPA3 | Chr12: 7753855-7756855 | DPPA3, Chr12: 7753855-7756855 |
| UMODL1 | Chr21: 42362994-42365994 | UMODL1, Chr21: 42362994-42365994 |
| LINCR | Chr2: 96533321-96536321 | LINCR, Chr2: 96533321-96536321 |
| S1PR5 | Chr19: 10487612-10490612 | S1PR5, Chr19: 10487612-10490612 |
| C1orf130 | Chr1: 24753688-24756688 | C1orf130, Chr1: 24753688-24756688 |
| TAS2R5 | Chr7: 141134985-141137985 | TAS2R5, Chr7: 141134985-141137985 |
| THOC5 | Chr22: 28278236-28281236 | THOC5, Chr22: 28278236-28281236 |
| MIP | Chr12: 55133196-55136196 | MIP, Chr12: 55133196-55136196 |

TABLE 5

| | | Methylation frequency: | | | | | | | | |
|---|---|---|---|---|---|---|---|---|---|---|
| | | >0-20% | 20-40% | 40-60% | 60-80% | 80-100% | | | | |
| | | M-NGS | | | | | | | | |
| | | | Benign | Localized | Metastatic | | | | | |
| | | Transplant | Adjacent | Prostate | Prostate | Cell Line - | Cell Line - | Cell Line - | | |
| S. No | Gene | Normal | Normal | Cancer | Cancer | PrEC-MNGS | LNCaP-MNGS | LNCaP-MEDIP | Ref | Description |
| 1 | PDLIM4 | 100% | 100% | 100% | 100% | 0% | 100% | 100% | Pubmeth | promoter |
| 2 | RBP1 | 0% | 0% | 40% | 75% | 0% | 100% | 100% | Pubmeth | promoter |
| 3 | RARRES1 | 0% | 17% | 40% | 75% | 0% | 100% | 100% | Pubmeth | promoter |
| 4 | CYP1A1 | 0% | 33% | 60% | 75% | 0% | 100% | 100% | Pubmeth | promoter |
| 5 | CDKN1C | 100% | 33% | 60% | 75% | 100% | 100% | 100% | Pubmeth | promoter |
| 6 | MGMT | 50% | 83% | 60% | 75% | 0% | 100% | 100% | Pubmeth | promoter |
| 7 | HIC1 | 100% | 83% | 60% | 75% | 0% | 100% | 100% | Pubmeth | promoter |
| 8 | PRIMA1 | 100% | 83% | 100% | 75% | 100% | 100% | 100% | Pubmeth | promoter |
| 9 | TNFRSF10C | 0% | 17% | 20% | 50% | 100% | 100% | 100% | Pubmeth | promoter |
| 10 | MT1G | 0% | 17% | 20% | 50% | 0% | 100% | 100% | Pubmeth | promoter |
| 11 | ESR2 | 0% | 0% | 40% | 50% | 0% | 100% | 100% | Pubmeth | promoter |
| 12 | RUNX3 | 0% | 0% | 40% | 50% | 0% | 100% | 100% | Pubmeth | promoter |
| 13 | LAMB3 | 50% | 17% | 40% | 50% | 0% | 100% | 100% | Pubmeth | promoter |
| 14 | HLTF | 0% | 33% | 40% | 50% | 0% | 100% | 100% | Pubmeth | promoter |
| 15 | GSTP1 | 0% | 33% | 60% | 50% | 0% | 100% | 100% | Pubmeth | promoter |
| 16 | RASSF1 | 0% | 33% | 60% | 50% | 0% | 100% | 100% | Pubmeth | promoter |
| 17 | SFN | 50% | 50% | 80% | 50% | 0% | 100% | 100% | Pubmeth | promoter |
| 18 | WRN | 0% | 0% | 0% | 25% | 0% | 100% | 0% | Pubmeth | promoter |
| 19 | S100A2 | 0% | 0% | 20% | 25% | 0% | 100% | 0% | Pubmeth | promoter |
| 20 | S100A6 | 0% | 0% | 20% | 25% | 0% | 100% | 100% | Pubmeth | promoter |
| 21 | LAMC2 | 0% | 0% | 20% | 25% | 0% | 100% | 100% | Pubmeth | promoter |
| 22 | LAMA3 | 0% | 17% | 20% | 25% | 0% | 100% | 100% | Pubmeth | promoter |
| 23 | PYCARD | 0% | 0% | 40% | 25% | 0% | 100% | 100% | Pubmeth | promoter |
| 24 | BCL2 | 0% | 0% | 40% | 25% | 0% | 100% | 100% | Pubmeth | promoter |
| 25 | CD44 | 0% | 17% | 40% | 25% | 0% | 100% | 100% | Pubmeth | promoter |
| 26 | APC | 0% | 0% | 60% | 25% | 0% | 100% | 100% | Pubmeth | promoter |
| 27 | GPX3 | 0% | 0% | 60% | 0% | 0% | 100% | 100% | Pubmeth | promoter |
| 28 | TMEFF2 | 0% | 0% | 0% | 50% | 0% | 0% | 0% | Pubmeth | promoter |
| 29 | NEUROG1 | 0% | 17% | 20% | 50% | 0% | 0% | 100% | Pubmeth | promoter |
| 30 | DKK3 | 0% | 33% | 20% | 50% | 0% | 0% | 100% | Pubmeth | promoter |
| 31 | EDNRB | 0% | 0% | 40% | 50% | 0% | 0% | 0% | Pubmeth | promoter |
| 32 | TNFRSF10D | 0% | 17% | 0% | 25% | 0% | 0% | 0% | Pubmeth | promoter |
| 33 | CDH13 | 0% | 0% | 20% | 25% | 0% | 0% | 0% | Pubmeth | promoter |
| 34 | ESR1 | 0% | 0% | 40% | 25% | 0% | 0% | 0% | Pubmeth | promoter |
| 35 | SFRP1 | 0% | 0% | 40% | 25% | 0% | 0% | 0% | Pubmeth | promoter |
| 36 | SCGB3A1 | 100% | 17% | 60% | 25% | 100% | 0% | 100% | Pubmeth | promoter |
| 37 | C20orf103 | 0% | 0% | 20% | 50% | 0% | 0% | 100% | Kron et al | promoter |
| | | | | | | | | | 2008 | |
| 38 | HOXD9 | 100% | 33% | 60% | 75% | 100% | 0% | 100% | Kron et al | promoter |
| | | | | | | | | | 2008 | |
| 39 | IRX1 | 0% | 0% | 60% | 50% | 0% | 0% | 100% | Kron et al | promoter |
| | | | | | | | | | 2008 | |
| 40 | TFAP2A | 0% | 17% | 20% | 50% | 0% | 100% | 100% | Kron et al | promoter |
| | | | | | | | | | 2008 | |
| 41 | WT1 | 0% | 0% | 40% | 50% | 0% | 0% | 0% | Kron et al | promoter |
| | | | | | | | | | 2008 | |
| 42 | SIX6 | 50% | 17% | 60% | 75% | 0% | 100% | 100% | Kron et al | promoter |
| | | | | | | | | | 2008 | |
| 43 | HOXD4 | 100% | 67% | 40% | 75% | 100% | 0% | 100% | Kron et al | promoter |
| | | | | | | | | | 2008 | |
| 44 | TCF7L1 | 0% | 0% | 40% | 50% | 0% | 0% | 0% | Kron et al | promoter |
| | | | | | | | | | 2008 | |
| 45 | SHH | 0% | 33% | 60% | 75% | 0% | 100% | 0% | Kron et al | promoter |
| | | | | | | | | | 2008 | |
| 46 | VAX1 | 50% | 33% | 40% | 75% | 100% | 100% | 100% | Kron et al | promoter |
| | | | | | | | | | 2008 | |
| 47 | HOXD3 | 0% | 0% | 40% | 75% | 0% | 0% | 0% | Kron et al | promoter |
| | | | | | | | | | 2008 | |
| 48 | CACNA1G | 0% | 0% | 0% | 25% | 0% | 0% | 0% | Kron et al | promoter |
| | | | | | | | | | 2008 | |
| 49 | GP5 | 50% | 67% | 100% | 100% | 0% | 100% | 100% | Kron et al | promoter |
| | | | | | | | | | 2008 | |
| 50 | SSTR1 | 0% | 17% | 0% | 75% | 0% | 100% | 100% | Kron et al | promoter |
| | | | | | | | | | 2008 | |
| 51 | NKX2-2 | 0% | 0% | 40% | 25% | 0% | 0% | 0% | Kron et al | promoter |
| | | | | | | | | | 2008 | |
| 52 | HOXC11 | 0% | 17% | 0% | 25% | 0% | 100% | 100% | Kron et al | promoter |
| | | | | | | | | | 2008 | |
| 53 | LBX1 | 0% | 0% | 0% | 50% | 0% | 0% | 0% | Kron et al | promoter |
| | | | | | | | | | 2008 | |
| 54 | MNX1 | 0% | 0% | 40% | 50% | 0% | 100% | 100% | Kron et al | promoter |
| | | | | | | | | | 2008 | |
| 55 | GRM1 | 0% | 0% | 0% | 25% | 0% | 0% | 0% | Kron et al | promoter |
| | | | | | | | | | 2008 | |
| 56 | LHX9 | 0% | 0% | 40% | 100% | 0% | 100% | 100% | Kron et al | promoter |
| | | | | | | | | | 2008 | |
| 57 | TP73 | 100% | 100% | 100% | 100% | 100% | 100% | 100% | | Imprinted region |
| 58 | NAP1L5 | 100% | 67% | 80% | 50% | 100% | 100% | 100% | | Imprinted region |
| 59 | PLAGL1 | 100% | 100% | 100% | 100% | 100% | 100% | 100% | | Imprinted region |
| 60 | GRB10 | 100% | 100% | 100% | 100% | 100% | 100% | 100% | | Imprinted region |
| 61 | PEG10 | 100% | 100% | 100% | 100% | 100% | 100% | 100% | | Imprinted region |
| 62 | PPP1R9A | 0% | 100% | 100% | 75% | 100% | 100% | 100% | | Imprinted region |
| 63 | MEST | 100% | 100% | 100% | 100% | 100% | 100% | 100% | | Imprinted region |
| 64 | DLGAP2 | 100% | 100% | 100% | 100% | 100% | 100% | 100% | | Imprinted region |
| 65 | KCNK9 | 100% | 100% | 100% | 100% | 100% | 100% | 100% | | Imprinted region |
| 66 | INPP5F | 100% | 100% | 100% | 100% | 100% | 100% | 100% | | Imprinted region |
| 67 | IGF2 | 100% | 100% | 100% | 100% | 100% | 100% | 100% | | Imprinted region |
| 68 | IGF2AS | 100% | 67% | 80% | 75% | 100% | 100% | 100% | | Imprinted region |
| 69 | KCNQ1 | 100% | 100% | 100% | 100% | 100% | 100% | 100% | | Imprinted region |
| 70 | CDKN1C | 0% | 0% | 0% | 0% | 0% | 0% | 0% | | Imprinted region |
| 71 | OSBPL5 | 100% | 100% | 100% | 100% | 100% | 100% | 100% | | Imprinted region |
| 72 | RB1 | 100% | 100% | 100% | 100% | 100% | 100% | 100% | | Imprinted region |
| 73 | F7 | 100% | 100% | 100% | 100% | 100% | 100% | 100% | | Imprinted region |
| 74 | DLK1 | 100% | 83% | 100% | 100% | 100% | 0% | 100% | | Imprinted region |
| 75 | DIO3 | 50% | 83% | 60% | 100% | 0% | 0% | 0% | | Imprinted region |
| 76 | MKRN3 | 50% | 33% | 40% | 25% | 0% | 0% | 0% | | Imprinted region |
| 77 | MAGEL2 | 100% | 67% | 60% | 75% | 0% | 0% | 100% | | Imprinted region |
| 78 | NDN | 50% | 33% | 60% | 75% | 0% | 100% | 100% | | Imprinted region |
| 79 | ATP10A | 100% | 100% | 100% | 100% | 100% | 100% | 100% | | Imprinted region |
| 80 | TCEB3C | 100% | 50% | 60% | 50% | 100% | 0% | 100% | | Imprinted region |
| 81 | PEG3 | 100% | 83% | 80% | 100% | 100% | 100% | 100% | | Imprinted region |
| 82 | HM13 | 100% | 100% | 100% | 100% | 100% | 100% | 100% | | Imprinted region |
| 83 | BLCAP | 100% | 100% | 100% | 75% | 100% | 100% | 100% | | Imprinted region |
| 84 | NNAT | 100% | 33% | 100% | 75% | 100% | 100% | 100% | | Imprinted region |
| 85 | GNAS | 100% | 100% | 100% | 100% | 100% | 100% | 100% | | Imprinted region |

TABLE 6

| | | Gene | | | | LNCaP H3K4 Binding | LNCaP DNA |
|---|---|---|---|---|---|---|---|
| TSS1 | TSS2 | Symbol | Strand | Name | TSS-1stExon | Location | Methylation |
| H3K4 | M | AK5 | + | NM_174858 | 1: 77520329-77520646 | 1: 77519901-77520750 | |
| | | | | NM_012093 | 1: 77520874-77521884 | | 1: 77520826-77521100 |
| H3K4 | M | APC | + | NM_001127511 | 5: 112071116-112071478 | 5: 112070726-112072125 | |
| | | | | NM_000038 | 5: 112101454-112101521 | | 5: 112101301-112101625 |
| H3K4 | M/H3K4 | ARRDC2 | + | NM_001025604 | 19: 17972943-17973382 | 19: 17972926-17973825 | |
| | | | | NM_015683 | 19: 17979976-17980393 | 19: 17980126-17980275 | 19: 17980226-17980750 |
| H3K4 | M/H3K4 | C1orf183 | − | NM_198926 | 1: 112099845-112101442 | 1: 112098976-112101100 | |
| | | | | NM_019099 | 1: 112083330-112085045 | 1: 112082476-112084050 | 1: 112084551-112084700 |
| H3K4 | M | CDC14B | − | NM_003671 | 9: 98421321-98423433 | 9: 98420701-98421475 | |
| | | | | NM_001077181 | 9: 98368870-98370524 | | 9: 98368576-98368775 |
| H3K4 | M | DUSP4 | − | NM_001394 | 8: 29263281-29265604 | 8: 29261426-29264150 | |
| | | | | NM_057158 | 8: 29261226-29263741 | | 8: 29261301-29261775 |
| H3K4 | M | EGLN2 | + | NM_080732 | 19: 45996887-45997007 | 19: 45996901-45998000 | |
| | | | | NM_053046 | 19: 45998020-45999160 | | 19: 45998151-45998575 |
| H3K4 | M | FAM102A | − | NM_001035254 | 9: 129782091-129783816 | 9: 129781076-129783225 | |
| | | | | NM_203305 | 9: 129752522-129754314 | | 9: 129752576-129752900 |
| H3K4 | M | GPR56 | + | NM_005682 | 16: 56211458-56211549 | 16: 56210651-56212700 | |
| | | | | NM_201525 | 16: 56220022-56220215 | | 16: 56219301-56220000 |
| H3K4 | M | HAS3 | + | NM_138612 | 16: 67697660-67697720 | 16: 67696951-67698350 | |
| | | | | NM_005329 | 16: 67698943-67699099 | | 16: 67698726-67699000 |
| H3K4 | M | HOXC4 | + | NM_014620 | 12: 52696908-52697465 | 12: 52696976-52698975 | |
| | | | | NM_153633 | 12: 52733927-52734412 | | 12: 52733876-52734225 |
| H3K4 | M | IRF7 | − | NM_001572 | 11: 605912-607499 | 11: 605601-606075 | |
| | | | | NM_004031 | 11: 605096-607228 | | 11: 606651-606775 |
| H3K4 | M | LRDD | − | NM_018494 | 11: 795178-796745 | 11: 795151-795725 | |
| | | | | NM_145887 | 11: 794093-795964 | | 11: 793951-794500 |
| H3K4 | M | LRRC20 | − | NM_018205 | 10: 71812310-71813888 | 10: 71811251-71812425 | |
| | | | | NM_207119 | 10: 71811261-71812920 | | 10: 71811301-71811450 |
| H3K4 | M | MAFG | − | NM_002359 | 17: 77478694-77480379 | 17: 77477226-77480175 | |
| | | | | NM_032711 | 17: 77474521-77476201 | | 17: 77474676-77474875 |
| H3K4 | M | MGAT1 | − | NM_001114617 | 5: 180169645-180171243 | 5: 180169026-180170900 | |
| | | | | NM_001114620 | 5: 180163207-180165008 | | 5: 180163051-180163500 |
| H3K4 | M | OSBPL9 | + | NM_148906 | 1: 51855351-51855481 | 1: 51855226-51856275 | |
| | | | | NM_148905 | 1: 51968080-51968384 | | 1: 51968251-51968550 |
| H3K4 | M | PILRB | + | NM_175047 | 7: 99771672-99771896 | 7: 99771051-99772125 | |
| | | | | NM_178238 | 7: 99793561-99793925 | | 7: 99793651-99794100 |
| H3K4 | M | RAPGEF3 | − | NM_001098531 | 12: 46438600-46440452 | 12: 46438651-46439800 | |
| | | | | NM_001098532 | 12: 46437915-46439948 | | 12: 46437776-46438075 |
| H3K4 | M | WDR5 | + | NM_017588 | 9: 135991030-135991143 | 9: 135989501-135992350 | |
| | | | | NM_052821 | 9: 135994734-135994901 | | 9: 135994751-135994925 |
| H3K4 | M | WWP2 | + | NM_199423 | 16: 68353774-68353795 | 16: 68353376-68354700 | |
| | | | | NM_199424 | 16: 68516401-68516888 | | 16: 68516251-68516750 |
| M/H3K4 | H3K4 | C14orf159 | + | NM_024952 | 14: 90650109-90650625 | 14: 90650001-90651300 | 14: 90650051-90650400 |
| | | | | NM_001102367 | 14: 90650731-90651091 | 14: 90650001-90651300 | |
| M | H3K4 | ADPRHL1 | − | NM_138430 | 13: 113155539-113157340 | | 13: |
| | | | | | | | 113155976-113156300 |
| | | | | NM_199162 | 13: 113151314-113152958 | 13: 113150826-113151525 | |
| M | H3K4 | ARTN | + | NM_057091 | 1: 44171578-44172144 | | 1: 44171576-44171725 |
| | | | | NM_003976 | 1: 44173617-44173975 | 1: 44173951-44175375 | |
| M | H3K4 | GNG4 | − | NM_001098721 | 1: 233880471-233882177 | | 1: 233880651-233880800 |
| | | | | NM_001098722 | 1: 233879584-233881416 | 1: 233879076-233880000 | |
| M | H3K4 | HYAL2 | − | NM_003773 | 3: 50335087-50336646 | | 3: 50334976-50335175 |
| | | | | NM_033158 | 3: 50333800-50335403 | 3: 50332851-50334450 | |
| M | H3K4 | MPG | + | NM_001015054 | 16: 67017-67124 | | 16: 67201-67825 |
| | | | | NM_001015052 | 16: 68168-68332 | 16: 67451-69250 | |
| M | H3K4 | NDRG2 | − | NM_201535 | 14: 20563675-20565275 | | 14: 20563926-20564225 |
| | | | | NM_201539 | 14: 20563027-20564604 | 14: 20562076-20563100 | |
| M | H3K4 | PDE4DIP | − | NM_022359 | 1: 143786986-143788936 | | 1: 143787051-143787525 |
| | | | | NM_001002811 | 1: 143641940-143644889 | 1: 143642476-143644625 | |
| M | H3K4 | RASSF1 | − | NM_170714 | 3: 50352990-50354871 | | 3: 50352651-50353000 |
| | | | | NM_170712 | 3: 50350577-50352168 | 3: 50348276-50350750 | |
| M | H3K4 | RUSC1 | + | NM_001105203 | 1: 153557263-153557406 | | 1: 153557251-153557675 |
| | | | | NM_001105205 | 1: 153560351-153560959 | 1: 153559451-153562075 | |
| M | H3K4 | SLC29A1 | + | NM_001078174 | 6: 44295219-44295497 | | 6: 44295051-44295225 |
| | | | | NM_001078176 | 6: 44299273-44299356 | 6: 44298901-44300250 | |
| M | H3K4 | TRIOBP | + | NM_001039141 | 22: 36423573-36423649 | | 22: 36423151-36423575 |
| | | | | NM_007032 | 22: 36472186-36472373 | 22: 36471201-36472100 | |
| M | H3K4 | TXNRD1 | + | NM_001093771 | 12: 103133688-103133801 | | 12: |
| | | | | | | | 103133526-103134225 |
| | | | | NM_003330 | 12: 103204856-103205254 | 12: 103204801-103206350 | |

| TSS1 | TSS2 | Gene Symbol | Strand | Name | CpG Islands | UCSC CpG Number | Length (bp) |
|---|---|---|---|---|---|---|---|
| H3K4 | M | AK5 | + | NM_174858 | 1: 77519902 | CpG: 103 | 910 |
| | | | | NM_012093 | 1: 77519902 | CpG: 103 | 910 |
| H3K4 | M | APC | + | NM_001127511 | 5: 112070978 | CpG: 64 | 838 |
| | | | | NM_000038 | | | |
| H3K4 | M/H3K4 | ARRDC2 | + | NM_001025604 | 19: 17972566 | CpG: 111 | 915 |
| | | | | NM_015683 | 19: 17978908 | CpG: 33 | 397 |
| H3K4 | M/H3K4 | C1orf183 | − | NM_198926 | 1: 112099564 | CpG: 89 | 951 |
| | | | | NM_019099 | 1: 112082575 | CpG: 170 | 2225 |
| H3K4 | M | CDC14B | − | NM_003671 | 9: 98420635 | CpG: 139 | 1422 |
| | | | | NM_001077181 | 9: 98368631 | CpG: 76 | 954 |
| H3K4 | M | DUSP4 | − | NM_001394 | 8: 29261583 | CpG: 344 | 4183 |
| | | | | NM_057158 | 8: 29261583 | CpG: 344 | 4183 |
| H3K4 | M | EGLN2 | + | NM_080732 | 19: 45996307 | CpG: 51 | 583 |
| | | | | NM_053046 | 19: 45996307 | CpG: 51 | 583 |
| H3K4 | M | FAM102A | − | NM_001035254 | 9: 129782336 | CpG: 108 | 971 |
| | | | | NM_203305 | | | |
| H3K4 | M | GPR56 | + | NM_005682 | | | |
| | | | | NM_201525 | | | |
| H3K4 | M | HAS3 | + | NM_138612 | 16: 67697129 | CpG: 190 | 2272 |
| | | | | NM_005329 | 16: 67697129 | CpG: 190 | 2272 |
| H3K4 | M | HOXC4 | + | NM_014620 | | | |
| | | | | NM_153633 | 12: 52734011 | CpG: 30 | 347 |
| H3K4 | M | IRF7 | − | NM_001572 | 11: 604760 | CpG: 119 | 1308 |
| | | | | NM_004031 | 11: 604760 | CpG: 119 | 1308 |
| H3K4 | M | LRDD | − | NM_018494 | 11: 794827 | CpG: 84 | 781 |
| | | | | NM_145887 | 11: 794827 | CpG: 84 | 781 |
| H3K4 | M | LRRC20 | − | NM_018205 | 10: 71811566 | CpG: 102 | 1077 |
| | | | | NM_207119 | 10: 71811566 | CpG: 102 | 1077 |
| H3K4 | M | MAFG | − | NM_002359 | 17: 77477970 | CpG: 184 | 2405 |
| | | | | NM_032711 | | | |
| H3K4 | M | MGAT1 | − | NM_001114617 | 5: 180169598 | CpG: 115 | 1217 |
| | | | | NM_001114620 | | | |
| H3K4 | M | OSBPL9 | + | NM_148906 | | | |
| | | | | NM_148905 | 1: 51967616 | CpG: 69 | 793 |
| H3K4 | M | PILRB | + | NM_175047 | 7: 99771174 | CpG: 84 | 849 |
| | | | | NM_178238 | | | |
| H3K4 | M | RAPGEF3 | − | NM_001098531 | 12: 46438821 | CpG: 49 | 592 |
| | | | | NM_001098532 | 12: 46438821 | CpG: 49 | 592 |
| H3K4 | M | WDR5 | + | NM_017588 | 9: 135990288 | CpG: 129 | 1531 |
| | | | | NM_052821 | | | |
| H3K4 | M | WWP2 | + | NM_199423 | | | |
| | | | | NM_199424 | | | |
| M/H3K4 | H3K4 | C14orf159 | + | NM_024952 | | | |
| | | | | NM_001102367 | | | |
| M | H3K4 | ADPRHL1 | − | NM_138430 | | | |
| | | | | NM_199162 | | | |
| M | H3K4 | ARTN | + | NM_057091 | 1: 44171655 | CpG: 35 | 467 |
| | | | | NM_003976 | 1: 44171655 | CpG: 35 | 467 |
| M | H3K4 | GNG4 | − | NM_001098721 | 1: 233878464 | CpG: 203 | 2225 |
| | | | | NM_001098722 | 1: 233878464 | CpG: 203 | 2225 |
| M | H3K4 | HYAL2 | − | NM_003773 | | | |
| | | | | NM_033158 | 3: 50333382 | CpG: 94 | 1228 |
| M | H3K4 | MPG | + | NM_001015054 | | | |
| | | | | NM_001015052 | 16: 67448 | CpG: 127 | 1183 |
| M | H3K4 | NDRG2 | − | NM_201535 | 14: 20562575 | CpG: 122 | 1535 |
| | | | | NM_201539 | 14: 20562575 | CpG: 122 | 1535 |
| M | H3K4 | PDE4DIP | − | NM_022359 | 1: 143786840 | CpG: 41 | 362 |
| | | | | NM_001002811 | 1: 143642696 | CpG: 116 | 1287 |
| M | H3K4 | RASSF1 | − | NM_170714 | 3: 50352807 | CpG: 84 | 737 |
| | | | | NM_170712 | 3: 50349268 | CpG: 139 | 1365 |
| M | H3K4 | RUSC1 | + | NM_001105203 | 1: 153557230 | CpG: 34 | 395 |
| | | | | NM_001105205 | 1: 153559093 | CpG: 221 | 2964 |
| M | H3K4 | SLC29A1 | + | NM_001078174 | 6: 44295164 | CpG: 24 | 214 |
| | | | | NM_001078176 | 6: 44299256 | CpG: 63 | 594 |
| M | H3K4 | TRIOBP | + | NM_001039141 | | | |
| | | | | NM_007032 | 22: 36471707 | CpG: 106 | 1238 |
| M | H3K4 | TXNRD1 | + | NM_001093771 | 12: 103133527 | CpG: 80 | 775 |
| | | | | NM_003330 | 12: 103204867 | CpG: 29 | 359 |

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

We claim:

1. A method of screening for the presence of prostate cancer in a subject, comprising
(a) contacting a biological sample from a subject with a reagent for detecting the methylation status of one or more genes selected from the group consisting of WFDC2, MAGI2, MEIS2, NTN4, GPRC5B, C9orf125, FGFR2, AOX1, VAMP5, C14orf159, PPP1R3C, S100A16 and AMT; and
(b) detecting the methylation status of said genes using an in vitro assay,
wherein an increased degree of methylation of said gene in said sample relative to the level of methylation of said gene in normal prostate cells in indicative of prostate cancer in said subject.

2. The method of claim 1, wherein the sample is selected from the group consisting of tissue, blood, plasma, serum, urine, urine supernatant, urine cell pellet, semen, prostatic secretions and prostate cells.

3. The method of claim 1, wherein detection is carried out utilizing Methylplex-Next Generation Sequencing (M-NGS).

4. The method of claim 1, wherein said cancer is selected from the group consisting of localized prostate cancer and metastatic prostate cancer.

5. The method of claim 1, said methylation is detected in the 5′ untranslated region of said gene.

6. The method of claim 5, wherein said methylation is detected in the promoter region of said gene.

7. The method of claim 1, wherein expression of said genes is decreased when said increased level of methylation is present.
